(19) United States
(12) Patent Application Publication
(10) Pub. No.: US 2025/0301903 A1
(43) Pub. Date: Sep. 25, 2025

(54) ORGANIC COMPOUND, COMPOSITION, ORGANIC ELECTROLUMINESCENT DEVICE AND ELECTRONIC APPARATUS

(71) Applicant: Shaanxi Lighte Optoelectronics Material Co., Ltd., Xi'an (CN)

(72) Inventors: Tiantian MA, Xi'an (CN); Lei YANG, Xi'an (CN); Zhen FENG, Xi'an (CN)

(21) Appl. No.: 18/862,807

(22) PCT Filed: Oct. 10, 2023
(86) PCT No.: PCT/CN2023/123854

§ 371 Date: Nov. 4, 2024

(30) Foreign Application Priority Data
Jan. 11, 2023 (CN) 202310041920.7

(51) Int. Cl. H10K 85/60; C07B 59/00; C07D 403/04; C07D 403/10; C07D 405/14; C07D 409/14; C09K 11/02; H10K 50/11

(52) U.S. Cl. CPC H10K 85/654; C07B 59/002; C07D 403/04; C07D 403/10; C07D 405/14; C07D 409/14; C09K 11/02; H10K 85/6572; H10K 85/6574; H10K 85/6576; H10K 50/11

(57) ABSTRACT

The present disclosure belongs to the technical field of organic electroluminescence, and relates to an organic compound as well as an organic electroluminescent device and an electronic apparatus using the same. The organic compound has a structure as shown in a Formula 1, and using the organic compound in the organic electroluminescent device can improve the performance of the organic electroluminescent device remarkably.

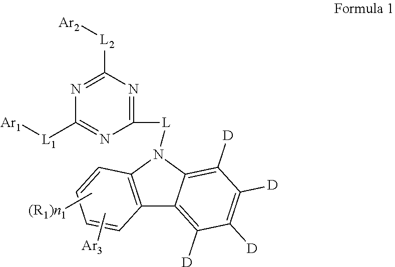

### CROSS REFERENCE TO RELATED APPLICATIONS

The present disclosure claims priority to the Chinese patent application with an application number CN202310041920.7 filed on Jan. 11, 2023, the entire content of the aforementioned Chinese patent application is hereby incorporated by reference as a part of the present disclosure.

### TECHNICAL FIELD

The present disclosure relates to the technical field of organic compounds, in particular to an organic compound as well as a composition, an organic electroluminescent device and an electronic apparatus including the same.

### BACKGROUND

With the development of electronic technologies and advances in material science, the application range of electronic elements for achieving electroluminescence is becoming increasingly wide. Such electronic elements typically include a cathode and an anode that are arranged oppositely, as well as a functional layer arranged between the cathode and the anode. The functional layer is composed of multiple organic or inorganic film layers, and generally includes an organic light-emitting layer, a hole transport layer located between the organic light-emitting layer and the anode, and an electron transport layer located between the organic light-emitting layer and the cathode. Taking an organic electroluminescent device as an example, it generally includes an anode, a hole transport layer, an organic light-emitting layer, an electron transport layer and a cathode stacked in sequence. When a voltage is applied to the anode and the cathode, an electric field is generated between the two electrodes, under the action of the electric field, electrons on the cathode side move towards the organic light-emitting layer, holes on the anode side also move towards the organic light-emitting layer. The electrons and the holes combine in the organic light-emitting layer to form excitons, which are in an excited state to release energy outwards, and thus the organic light-emitting layer is made to emit light outwards.

The prior art has disclosed a host material for preparing an organic light-emitting layer in an organic electroluminescent device. However, it is still necessary to continue developing novel materials to further improve the performance of electronic components.

### SUMMARY

To solve the above problems, an objective of the present disclosure is to provide an organic compound as well as a composition, an organic electroluminescent device and an electronic apparatus including the same. The organic compound may improve the performance of the organic electroluminescent device and the electronic apparatus, such as lowering a driving voltage of the device, and improving the efficiency and prolonging the lifetime of the device.

According to a first aspect of the present disclosure, an organic compound is provided, and the organic compound has a structure as shown in a Formula 1:

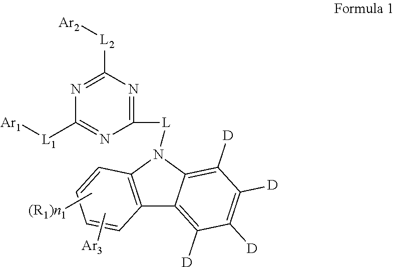

where Ar₁ and Ar₂ are the same or different, and are respectively and independently selected from a substituted or unsubstituted aryl with 6 to 30 carbon atoms, a substituted or unsubstituted dibenzofuranyl, and a substituted or unsubstituted dibenzothienyl;
L, L₁ and L₂ are the same or different, and are respectively and independently selected from a single bond, and a substituted or unsubstituted arylene with 6 to 30 carbon atoms;
Ar₃ is selected from a substituted or unsubstituted biphenyl;
substituent(s) in Ar₃ are each independently selected from a deuterium, a pentadeuterophenyl and a phenyl;
R₁ is a hydrogen or a deuterium;
n₁ is the number of R₁ and selected from 1, 2 or 3; and
substituent(s) in L, L₁, L₂, Ar₁ and Ar₂ are the same or different, and are respectively and independently selected from a deuterium, a cyano, a halogen group, an alkyl with 1 to 10 carbon atoms, a haloalkyl with 1 to 10 carbon atoms, a deuteroalkyl with 1 to 10 carbon atoms, an aryl with 6 to 20 carbon atoms, a deuteroaryl with 6 to 20 carbon atoms, a haloaryl with 6 to 20 carbon atoms, a heteroaryl with 5 to 20 carbon atoms, and a cycloalkyl with 3 to 10 carbon atoms.

According to a second aspect of the present disclosure, a composition is provided, and the composition contains the organic compound provided by the first aspect of the present disclosure and a second compound shown in a Formula 2:

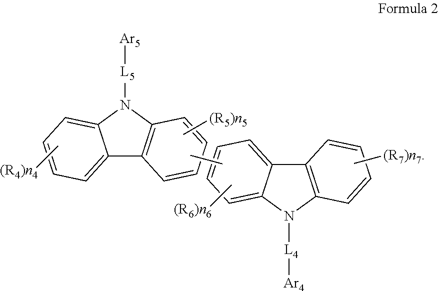

According to a third aspect of the present disclosure, an organic electroluminescent device is provided, including an anode and a cathode arranged oppositely, as well as a functional layer arranged between the anode and the cathode. The functional layer contains the organic compound disclosed in the first aspect of the present disclosure or the composition disclosed in the second aspect of the present disclosure.

According to a fourth aspect of the present disclosure, an electronic apparatus is provided, including the organic electroluminescent device disclosed in the third aspect of the present disclosure.

The present disclosure provides the organic compound, a core structure of the organic compound is that carbazole is in direct or indirect connection with a triazine group through a nitrogen atom, one of benzene rings on a carbazole ring is fully deuterated, while the other benzene ring is connected with substituted or unsubstituted biphenyl as a substituent. The presence of the substituted or unsubstituted biphenyl on the carbazole group not only expands the aromatic conjugation range of a molecular structure, but also lowers molecular symmetry, resulting in better energy transfer properties and reduced crystallinity of a material. In the present disclosure, a specific position of carbazolyl is deuterated, while the stability of the molecular structure is effectively improved, low molecular symmetry of molecules is kept, and thus the material's photoelectric stability and film-forming properties are further improved. The organic compound of the present disclosure has good carrier transport properties, energy transfer properties, and photoelectric stability, and is suitable for use as a host material for a light-emitting layer in the organic electroluminescent device. An organic electroluminescent device using the organic compound as the host material has significantly improved lifetime properties and high luminous efficiency while a low driving voltage is kept.

Other features and advantages of the present disclosure will be described in detail in the subsequent detailed description.

### BRIEF DESCRIPTION OF THE DRAWINGS

The accompanying drawings are intended to provide a further understanding of the present disclosure, form a part of the description, and are used to explain the present disclosure together with the following detailed description, but do not constitute a limitation to the present disclosure.

FIG. 1 is a schematic structural diagram of an organic electroluminescent device of the present disclosure.

FIG. 2 is a schematic structural diagram of an electronic apparatus of the present disclosure.

### REFERENCE NUMERALS

100, anode; 200, cathode; 300, functional layer; 310, hole injection layer; 320, hole transport layer; 330, hole auxiliary layer; 340, organic light-emitting layer; 350, electron transport layer; 360, electron injection layer; 400, first electronic apparatus.

### DETAILED DESCRIPTION

For the above problems existing in the prior art, an objective of the present disclosure is to provide an organic compound as well as an organic electroluminescent device containing the organic compound, and an electronic apparatus. The organic compound may improve the performance of the organic electroluminescent device and the electronic apparatus, such as lowering a driving voltage of a device, and improving the efficiency and prolonging the lifetime of the device.

According to a first aspect of the present disclosure, an organic compound is provided, and the organic compound has a structure as shown in a Formula 1:

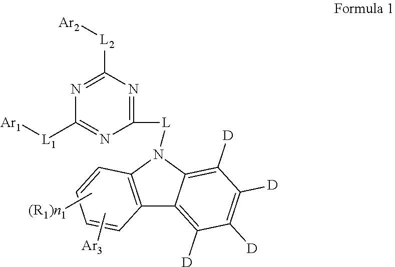

where Ar₁ and Ar₂ are the same or different, and are respectively and independently selected from a substituted or unsubstituted aryl with 6 to 30 carbon atoms, a substituted or unsubstituted dibenzofuranyl, and a substituted or unsubstituted dibenzothienyl;
L, L₁ and L₂ are the same or different, and are respectively and independently selected from a single bond, and a substituted or unsubstituted arylene with 6 to 30 carbon atoms;
Ar₃ is selected from a substituted or unsubstituted biphenyl;
substituent(s) in Ar₃ are selected from a deuterium, a pentadeuterophenyl and a phenyl;
R₁ is a hydrogen or a deuterium;
n₁ is the number of R₁ and selected from 1, 2 or 3; and
substituent(s) in L, L₁, L₂, Ar₁ and Ar₂ are the same or different, and are respectively and independently selected from a deuterium, a cyano, a halogen group, an alkyl with 1 to 10 carbon atoms, a haloalkyl with 1 to 10 carbon atoms, a deuteroalkyl with 1 to 10 carbon atoms, an aryl with 6 to 20 carbon atoms, a deuteroaryl with 6 to 20 carbon atoms, a haloaryl with 6 to 20 carbon atoms, a heteroaryl with 5 to 20 carbon atoms, and a cycloalkyl with 3 to 10 carbon atoms.

In the present disclosure, adopted description manners “each . . . independently”, “ . . . respectively and independently“and” . . . each independently” can be interchangeable and should be broadly understood. They can refer to that specific options expressed by the same symbols in different groups do not affect each other, or they can refer to that specific options expressed by the same symbols in the same group do not affect each other. For example, the meaning of

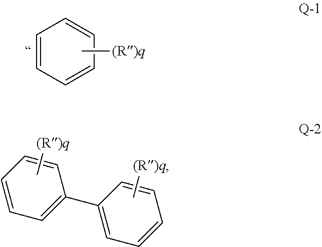

each q is independently 0, 1, 2 or 3, and each R″ is independently selected from hydrogen, deuterium, fluorine and chlorine” is as follows: a formula Q-1 represents that there are q substituents R″ on a benzene ring, each R″ may be the same or different, and the options of each R″ do not affect each other; and a formula Q-2 represents that there are q substituents R″ on each benzene ring of xenene, the numbers q of the R″ substituents on the two benzene rings may be the same or different, each R″ may be the same or different, and the options of each R″ do not affect each other.

In the present disclosure, such term “substituted or unsubstituted” refers to that functional groups recorded behind the term may or may not have substituents (for the convenience of description in the below, the substituents are collectively referred to as Rc). For example, “a substituted or unsubstituted aryl” refers to an aryl with a substituent Rc or an aryl without substitution. The above substituent Rc may be, for example, deuterium, cyano, a halogen group, alkyl, halogenated alkyl, haloalkyl, deuteroalkyl, aryl, deuteroaryl, haloaryl, heteroaryl, cycloalkyl, or the like. The number of the substituents may be one or more.

In the present disclosure, “a plurality of” refers to two or more, e.g., two, three, four, five, six, etc.

In the present disclosure, the number of carbon atoms of a substituted or unsubstituted functional group refers to the number of all carbon atoms. For example, if L₁ is a substituted arylene with 12 carbon atoms, then the number of all the carbon atoms of the arylene and a substituent on the arylene is 12.

In the present disclosure, aryl refers to any functional group or substituent derived from an aromatic carbon ring. Aryl may be monocyclic aryl (e.g., phenyl) or polycyclic aryl, in other words, aryl may be monocyclic aryl, fused-ring aryl, two or more monocyclic aryl connected through a carbon-carbon bond, monocyclic aryl and fused-ring aryl connected through a carbon-carbon bond, and two or more fused-ring aryl connected through a carbon-carbon bond. That is, unless otherwise stated, two or more aromatic groups conjugately connected through a carbon-carbon bond may also be considered as aryl in the present disclosure. The fused-ring aryl may, for example, include bicyclic fused aryl (e.g., naphthyl), tricyclic fused aryl (e.g., phenanthryl, fluorenyl and anthryl), and the like. Aryl does not contain heteroatoms such as B, N, O, S, P, Se and Si. Instances of aryl may include but not limited to phenyl, naphthyl, fluorenyl, anthryl, phenanthryl, biphenyl, terphenyl, triphenylene, perylenyl, benzo[9,10]phenanthryl, pyrenyl, benzofluoranthenyl, chrysenyl, spirobifluorenyl, etc. In the present disclosure, involved arylene refers to a divalent group formed by aryl further losing one hydrogen atom.

In the present disclosure, terphenyl includes

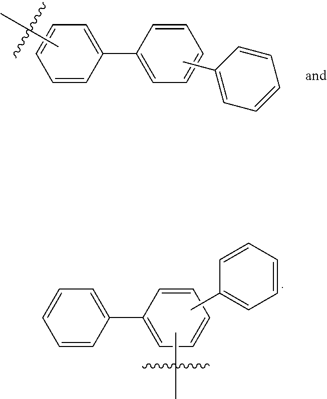

In the present disclosure, the number of carbon atoms of a substituted aryl refers to a total number of carbon atoms of aryl and a substituent on the aryl, for example, a substituted aryl with 18 carbon atoms refers to that the total number of the carbon atoms of the aryl and the substituent is 18.

In the present disclosure, the number of carbon atoms of a substituted or unsubstituted aryl may be 6, 10, 12, 13, 14, 15, 16, 17, 18, 20, 24, 25 or 30. In some embodiments, a substituted or unsubstituted aryl is a substituted or unsubstituted aryl with 6 to 30 carbon atoms, in some other implementations, a substituted or unsubstituted aryl is a substituted or unsubstituted aryl with 6 to 25 carbon atoms, in some other implementations, a substituted or unsubstituted aryl is a substituted or unsubstituted with 6 to 20 carbon atoms, and in some other implementations, a substituted or unsubstituted aryl is a substituted or unsubstituted aryl with 6 to 12 carbon atoms.

In the present disclosure, fluorenyl may be substituted by one or more substituents, where any two adjacent substituents may be bonded to each other to form a ring structure. In a case that the aforementioned fluorenyl is substituted, the substituted fluorenyl may be:

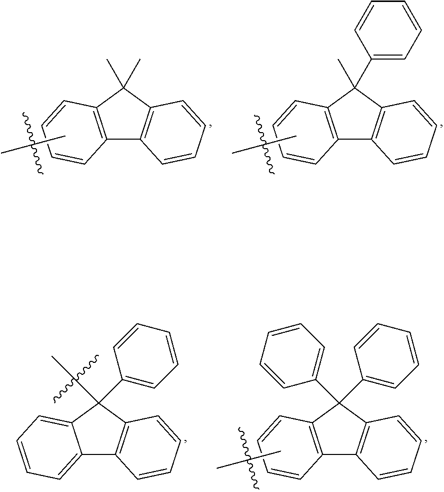

etc., but is not limited to this.

In the present disclosure, aryl used as a substituent of L, L₁, L₂, Ar₁ and Ar₂ is, for example, but not limited to, phenyl, naphthyl, etc.

In the present disclosure, heteroaryl refers to a univalent aromatic ring containing 1, 2, 3, 4, 5 or 6 heteroatoms in the ring or its derivatives, and the heteroatoms may be one or more of B, O, N, P, Si, Se and S. The heteroaryl may be monocyclic heteroaryl or polycyclic heteroaryl, in other words, the heteroaryl may be a single aromatic ring system, or a plurality of aromatic ring systems connected through carbon-carbon bonds, and any aromatic ring system is one aromatic monocycle or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, and N-phenylcarbazolyl, N-pyridylcarbazolyl, N-methylcarbazolyl, etc., and is not limited to this.

In the present disclosure, the number of carbon atoms of a substituted or unsubstituted heteroaryl may be selected from 5, 6, 7, 8, 9, 10, 11, 12, 13, 14, 15, 16, 17, 18, 20, 21, 22, 23, 24, 25, 26, 27, 28, 29 or 30. In some embodiments, a substituted or unsubstituted heteroaryl is a substituted or unsubstituted heteroaryl with 5 to 20 carbon atoms, and in some other implementations, a substituted or unsubstituted heteroaryl is a substituted or unsubstituted heteroaryl with 12 to 18 carbon atoms.

In the present disclosure, a substituted heteroaryl may be a heteroaryl with one or two or more hydrogen atoms being substituted by groups such as a deuterium atom, a halogen group, a cyano, an aryl, a heteroaryl, a trialkylsilyl, an alkyl, a cycloalkyl and a haloalkyl. It is to be understood that, the number of carbon atoms of the substituted heteroaryl refers to a total number of carbon atoms of heteroaryl and a substituent on the heteroaryl.

In the present disclosure, an alkyl with 1 to 10 carbon atoms may include a linear alkyl with 1 to 10 carbon atoms and a branched alkyl with 3 to 10 carbon atoms. The number of carbon atoms of an alkyl may be, for example, 1, 2, 3, 4, 5, 6, 7, 8, 9 and 10, and specific instances of an alkyl include but are not limited to methyl, ethyl, n-propyl, isopropyl, n-butyl, isobutyl, tert-butyl, n-pentyl, isopentyl, neopentyl, n-hexyl, etc.

In the present disclosure, the halogen group may be, for example, fluorine, chlorine, bromine and iodine.

In the present disclosure, specific instances of trialkylsilyl include but are not limited to trimethylsilyl, etc.

In the present disclosure, specific instances of halogenated alkyl include but are not limited to trifluoromethyl.

In the present disclosure, specific instances of deuterated alkyl include but are not limited to trideuteromethyl.

In the present disclosure, the number of carbon atoms of cycloalkyl with 3 to 10 carbon atoms may be, for example, 3, 4, 5, 6, 7, 8 or 10. Specific instances of cycloalkyl include but are not limited to cyclopentyl, cyclohexyl, and adamantyl.

In the present disclosure, a single bond

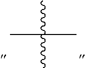

extending from a ring system involved in a non-positioning connecting bond represents that one end of the connecting bond may be connected to any position in the ring system through which the bond penetrates, and the other end may be connected to the rest of a compound molecule. For example, as shown in Formula (f) below, naphthyl represented by Formula (f) is connected to other positions of the molecule through two non-positioning connecting bonds that penetrate through a bicyclic ring, and its meaning includes any possible connection manner as shown in Formula (f-1) to Formula (f-10).

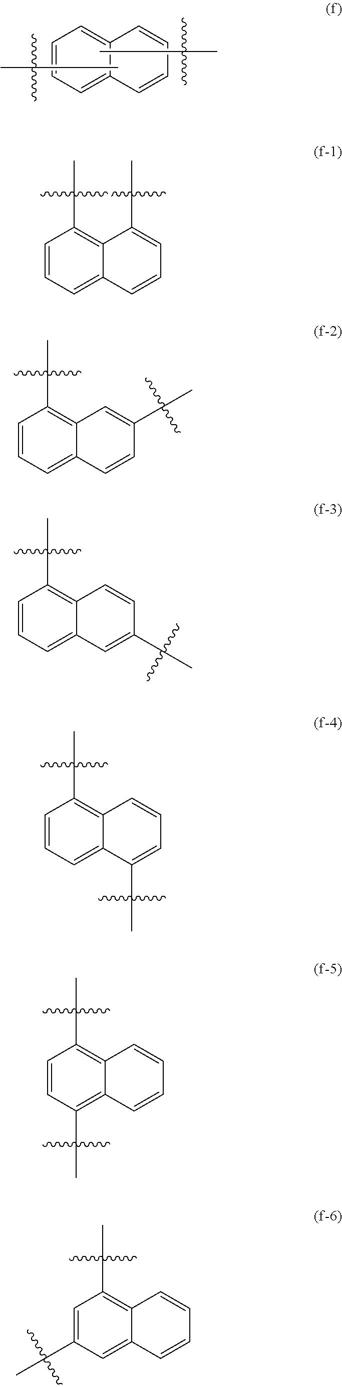

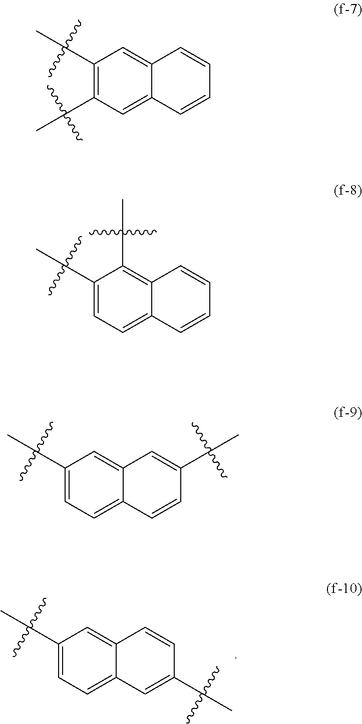 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in Formula (X′) below, dibenzofuryl represented by Formula (X′) is connected to other positions of the molecule through one non-positioning connecting bond that extends out of the middle of a benzene ring on one side, and its meaning includes any possible connection manner as shown in Formula (X′-1) to Formula (X′-4).

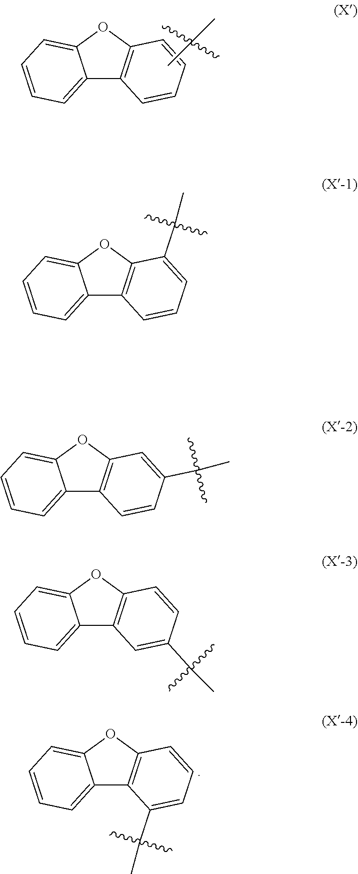

In some implementations of the present disclosure, the organic compound is selected from a compound shown in a Formula A, a Formula B, a Formula C or a Formula D:

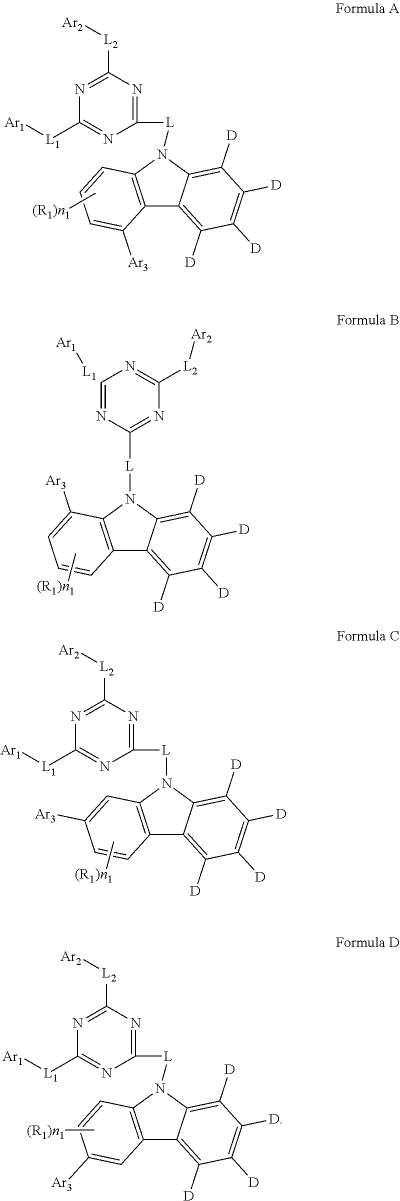

In some implementations of the present disclosure, the organic compound is selected from a compound shown in a Formula 1-1, a Formula 1-2, a Formula 1-3, a Formula 1-4, a Formula 1-5, a Formula 1-6, a Formula 1-7 or a Formula 1-8:

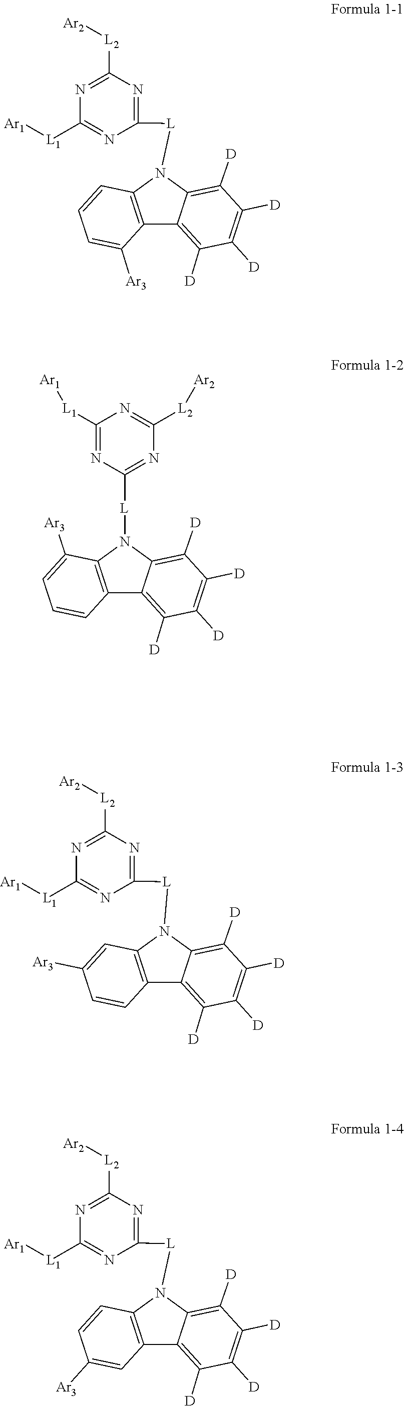

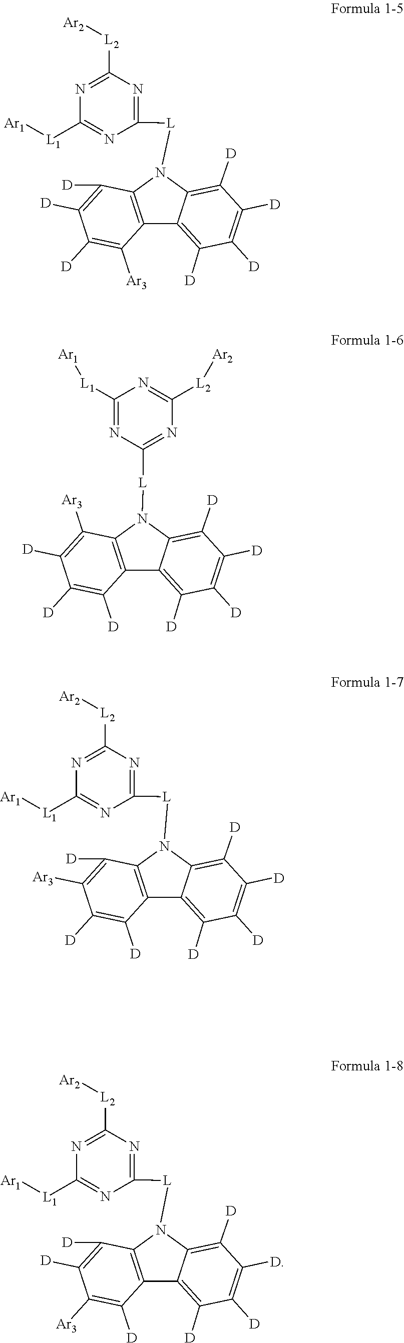

In some implementations of the present disclosure, Ar₃ is selected from a group consisting of the following groups:

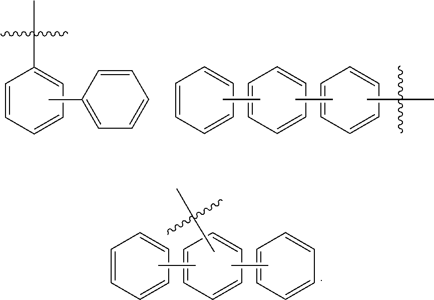

Specifically, Ar₃ is selected from a group consisting of the following groups:

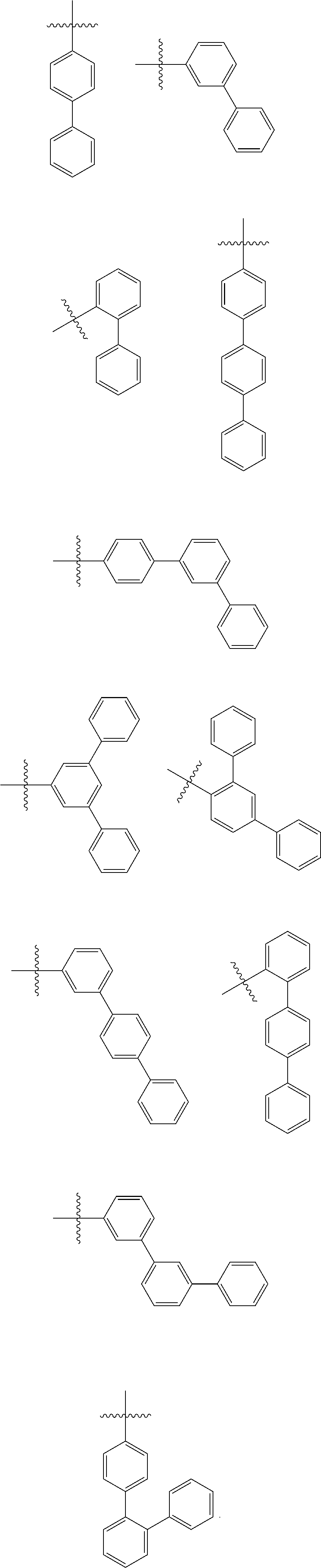

In some implementations of the present disclosure, L, L₁ and L₂ are the same or different, and are respectively and independently selected from a single bond, and a substituted or unsubstituted arylene with 6 to 12 carbon atoms.

Optionally, substituent(s) in L, L₁ and L₂ are the same or different and each independently selected from a deuterium, a halogen group, a cyano, an alkyl with 1 to 5 carbon atoms and a phenyl.

In some other implementations of the present disclosure, L, L₁ and L₂ are the same or different, and are respectively and independently selected from a single bond, a substituted or unsubstituted phenylene, a substituted or unsubstituted naphthylene and a substituted or unsubstituted biphenylene.

Optionally, L, L₁ and L₂ are the same or different, and are respectively and independently selected from a deuterium, a fluorine, a cyano, a methyl, an ethyl, a n-propyl, an isopropyl, a tert-butyl and a phenyl.

Further optionally, L, L₁ and L₂ are the same or different, and are respectively and independently selected from a single bond or a group consisting of the following groups:

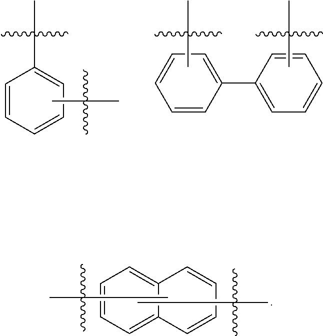

Specifically, L, L₁ and L₂ are the same or different, and are respectively and independently selected from a single bond or a group consisting of the following groups:

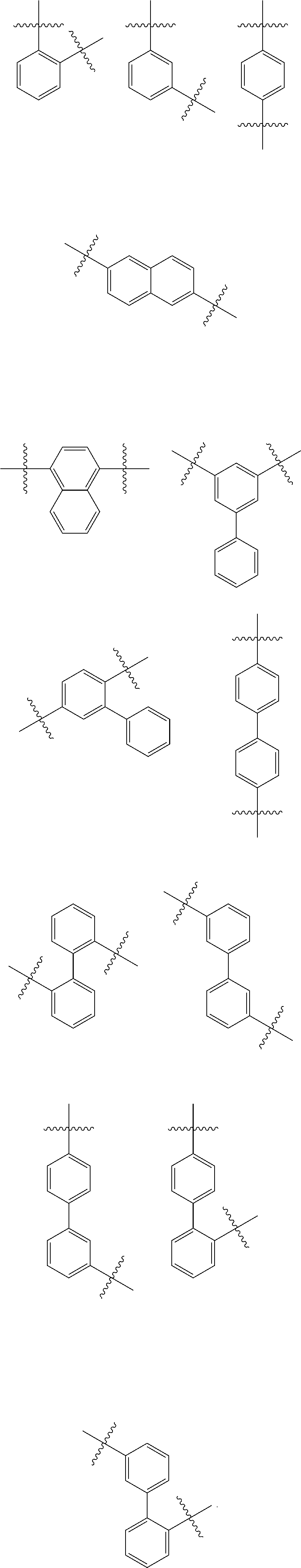

In some implementations of the present disclosure, Ar₁ and Ar₂ are the same or different, and are respectively and independently selected from a substituted or unsubstituted aryl with 6 to 20 carbon atoms, a substituted or unsubstituted dibenzofuranyl, and a substituted or unsubstituted dibenzothienyl.

Optionally, substituent(s) in Ar₁ and Ar₂ are the same or different, and are respectively and independently selected from a deuterium, a halogen group, a cyano, an alkyl with 1 to 5 carbon atoms, a phenyl and a pentadeuterophenyl.

In some other implementations of the present disclosure, Ar₁ and Ar₂ are the same or different, and are respectively and independently selected from a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted terphenyl, a substituted or unsubstituted dibenzofuranyl, and a substituted or unsubstituted dibenzothienyl.

Optionally, substituent(s) in Ar₁ and Ar₂ are the same or different and each independently selected from a deuterium, a fluorine, a cyano, a methyl, an ethyl, a n-propyl, an isopropyl, a tert-butyl, a phenyl and a pentadeuterophenyl.

In some other implementations of the present disclosure, Ar₁ and Ar₂ are the same or different and each independently selected from a substituted or unsubstituted group W, where the unsubstituted group W is selected from a group consisting of the following groups:

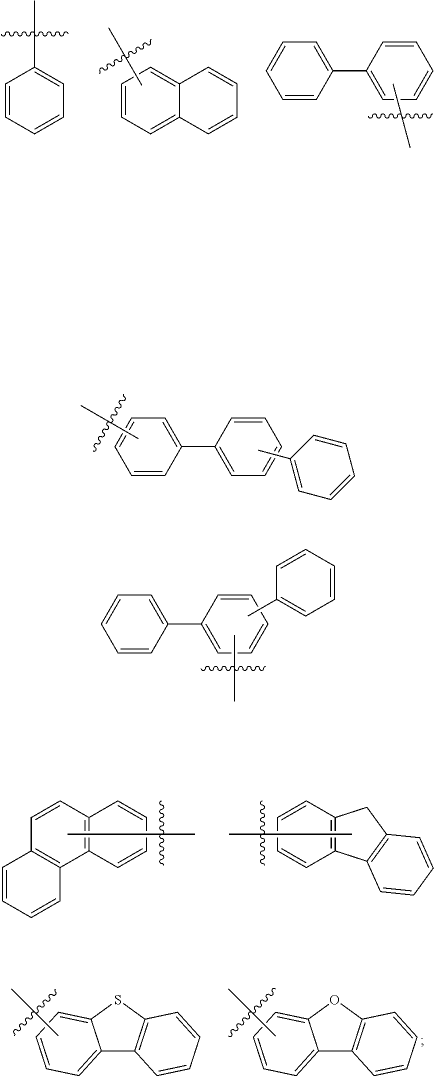

where

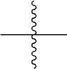

represents a chemical bond; and the substituted group W has one or two or more substituents, the substituents are each independently selected from a deuterium, a fluorine, a cyano, a methyl, an ethyl, a n-propyl, an isopropyl, a tert-butyl, a phenyl and a pentadeuterophenyl, and when the number of the substituents on the group W is greater than 1, the substituents are the same or different.

Optionally, Ar₁ and Ar₂ are the same or different, and are respectively and independently selected from a group consisting of the following groups:

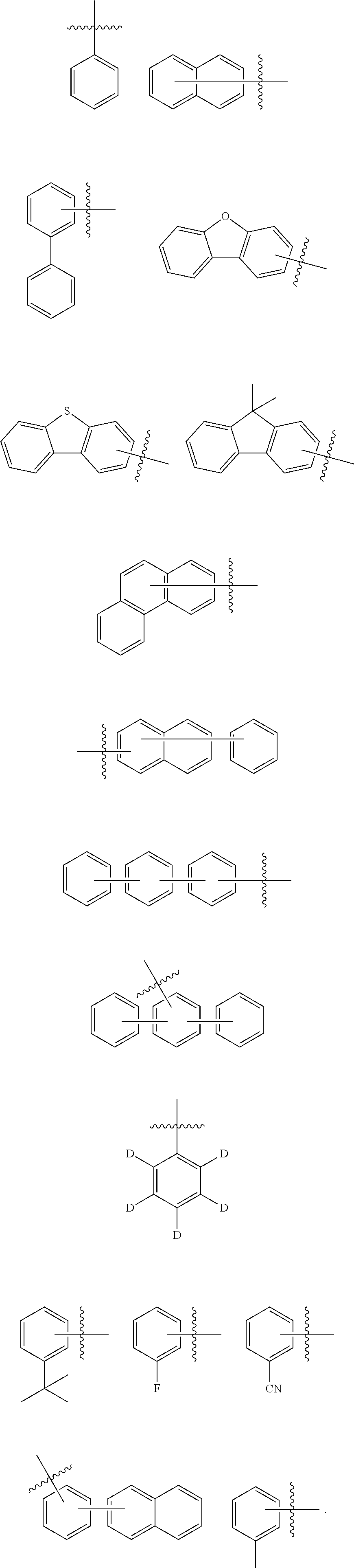

Specifically, Ar₁ and Ar₂ are the same or different, and are respectively and independently selected from a group consisting of the following groups:

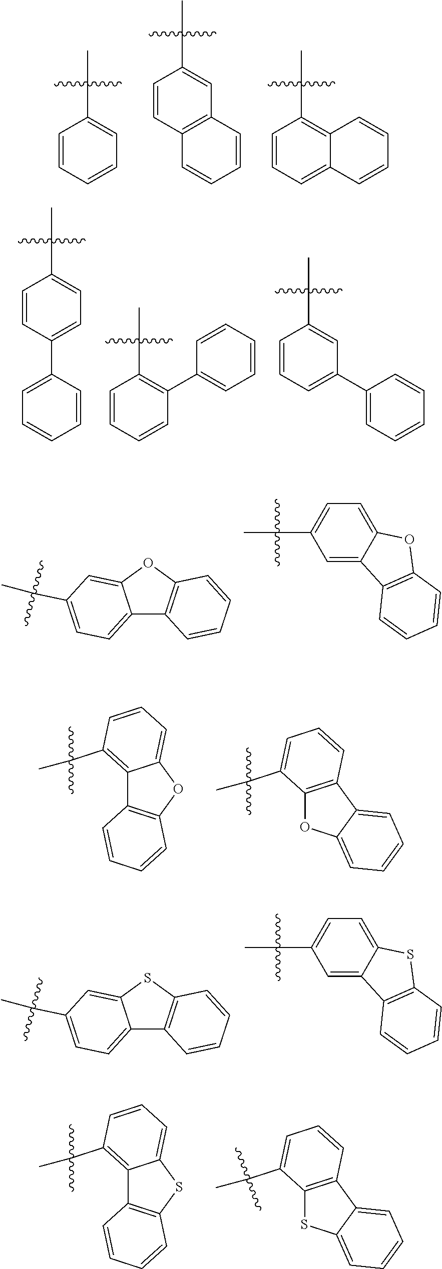

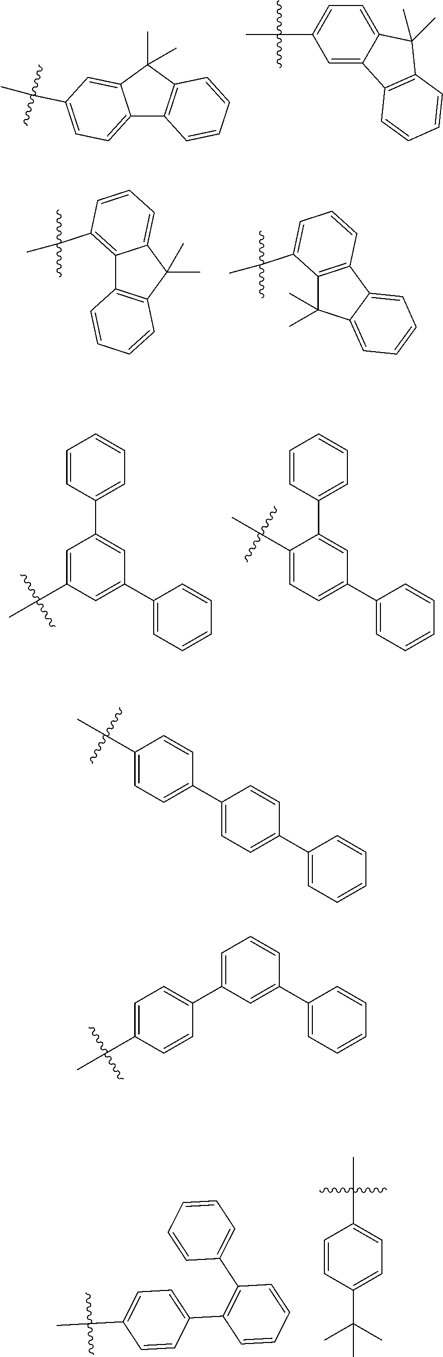

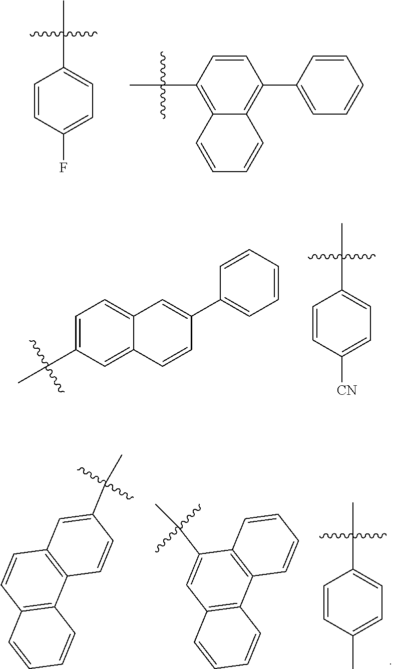

In some implementations of the present disclosure,

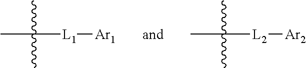

are the same or different, and are respectively and independently selected from a group consisting of the following groups:

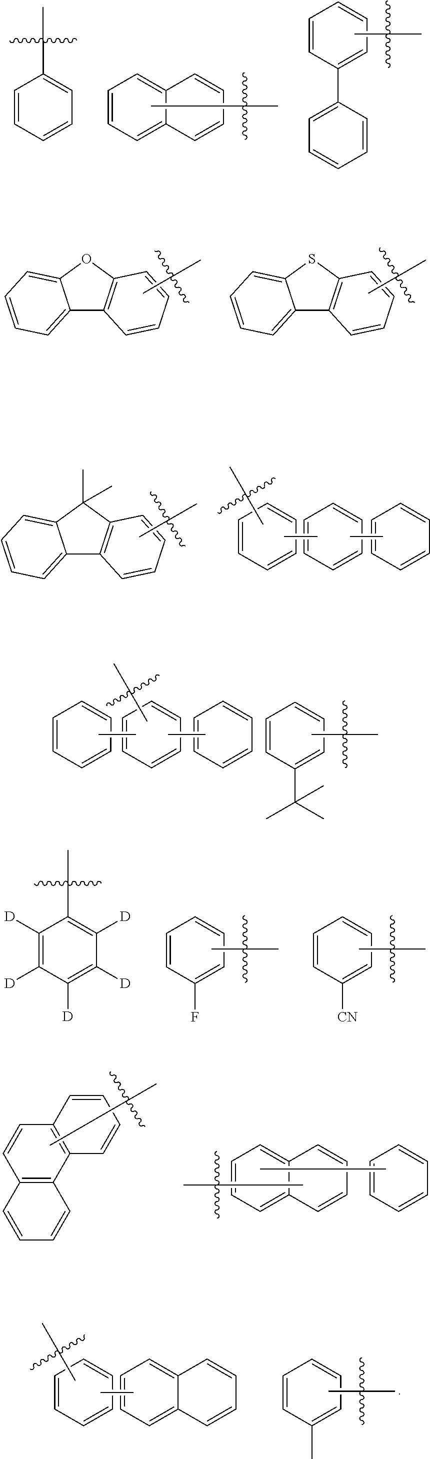

Specifically,

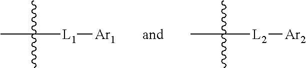

are the same or different, and are respectively and independently selected from a group consisting of the following groups:

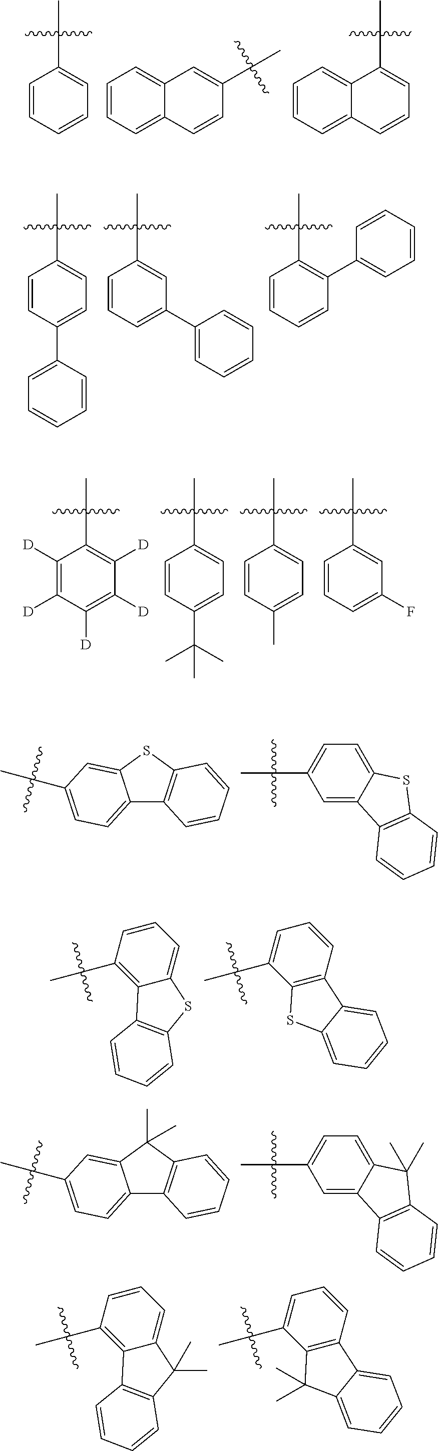

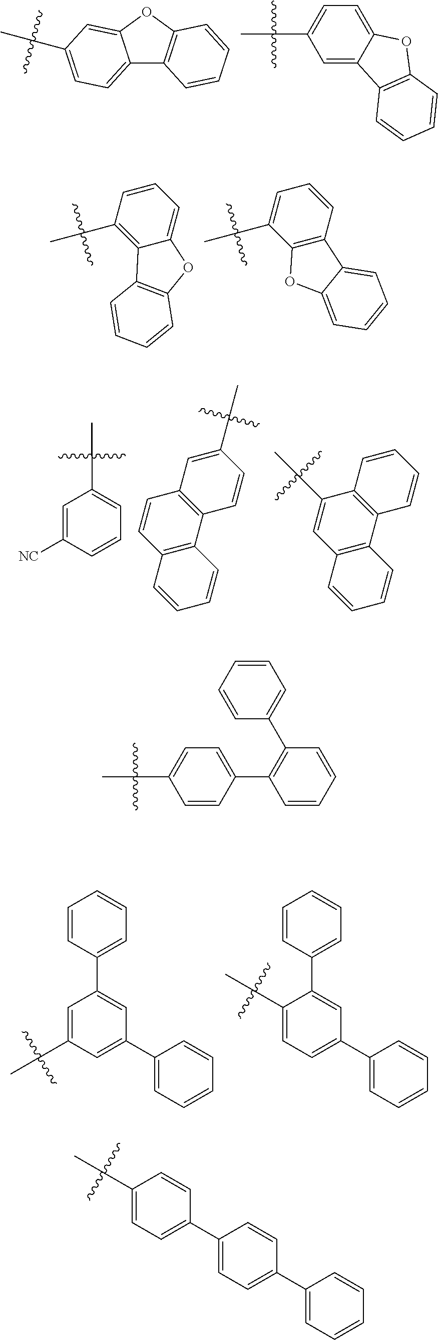

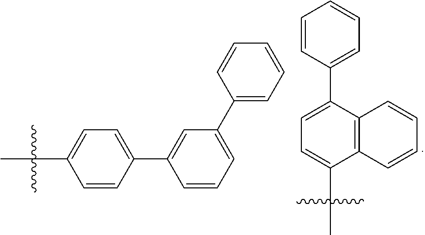

In some implementations of the present disclosure,

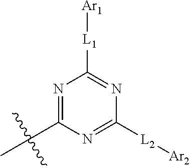

in the Formula 1 is selected from a group consisting of the following groups:

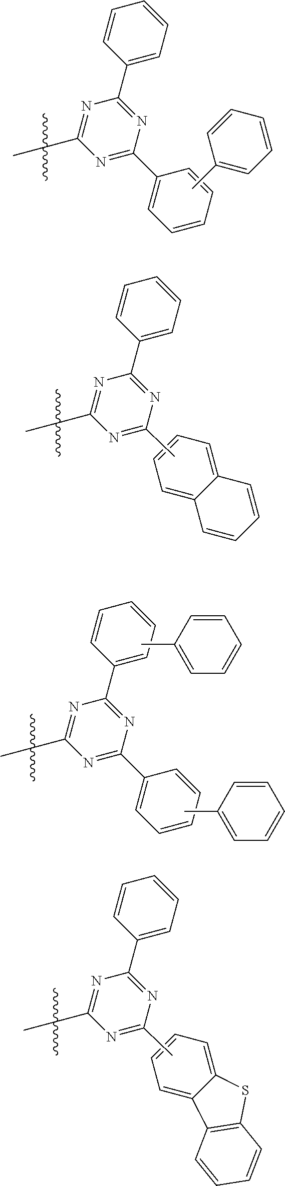

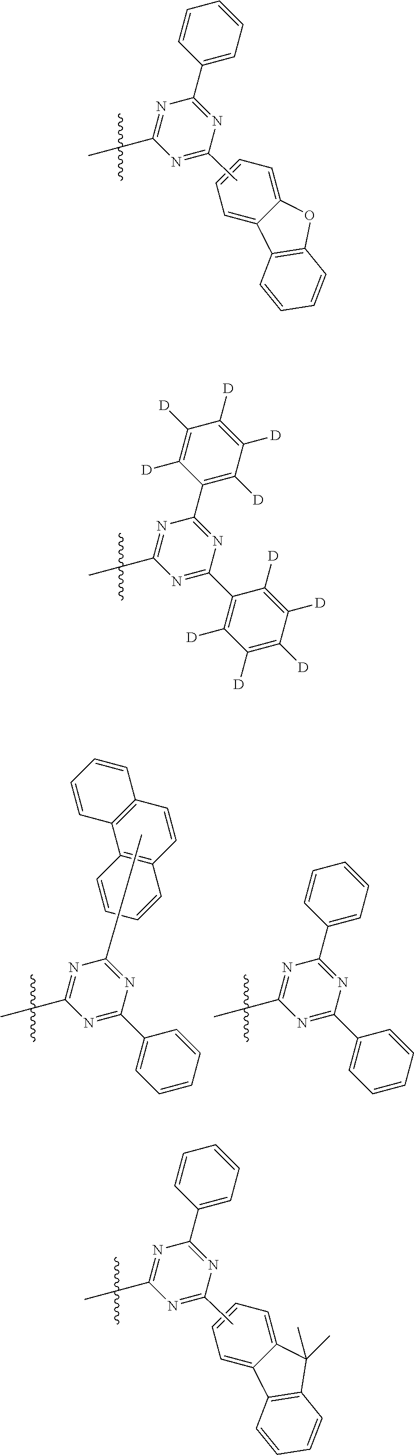

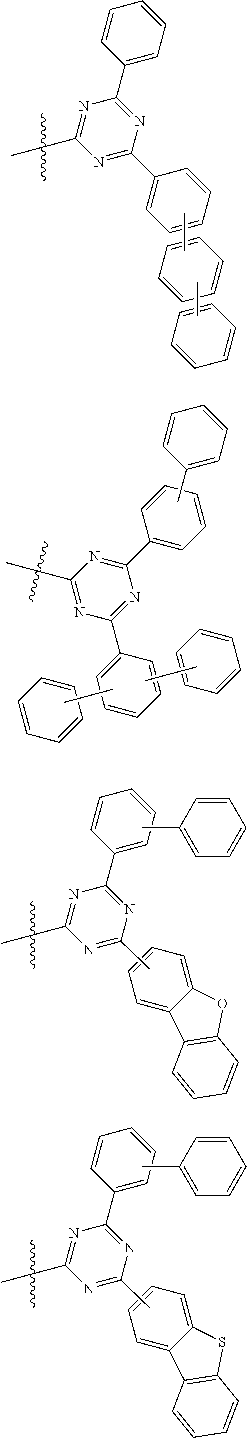

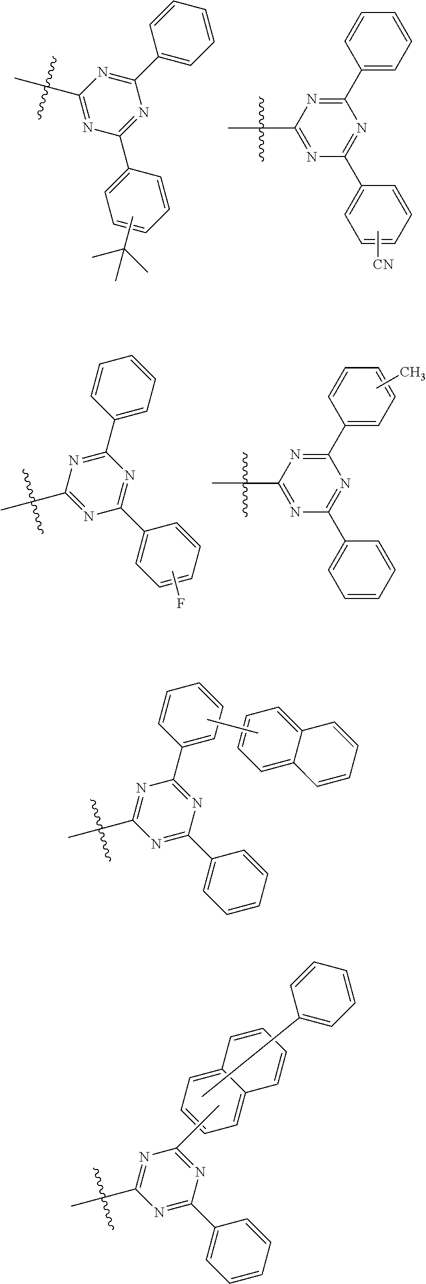

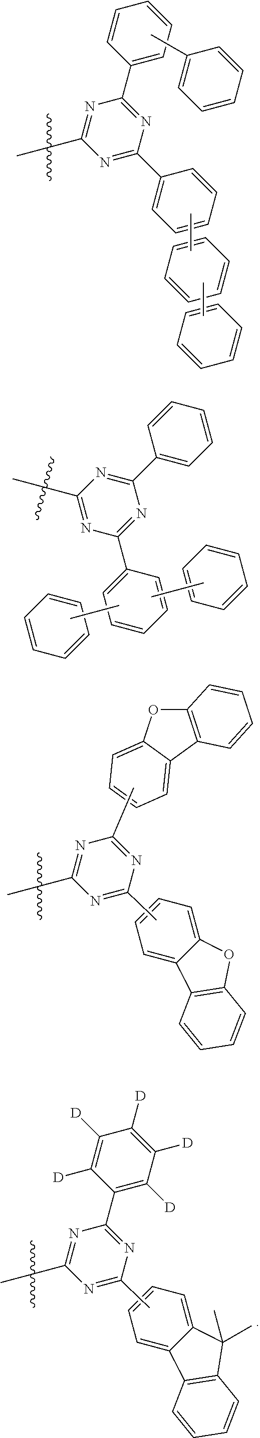

Specifically,

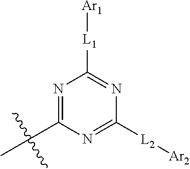

in Formula 1 is selected from a group consisting of the following groups:

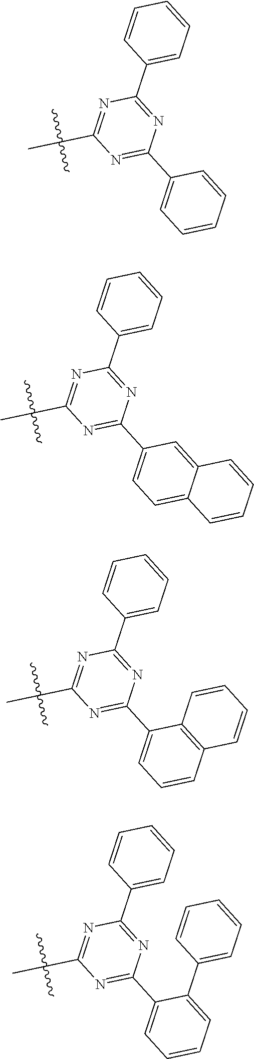

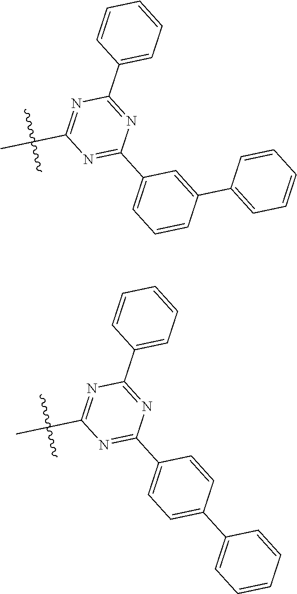 [SMILES: *c1nc(-c2ccccc2)nc(-c2cccc(-c3ccccc3)c2)n1 ; *c1nc(-c2ccccc2)nc(-c2ccc(-c3ccccc3)cc2)n1]

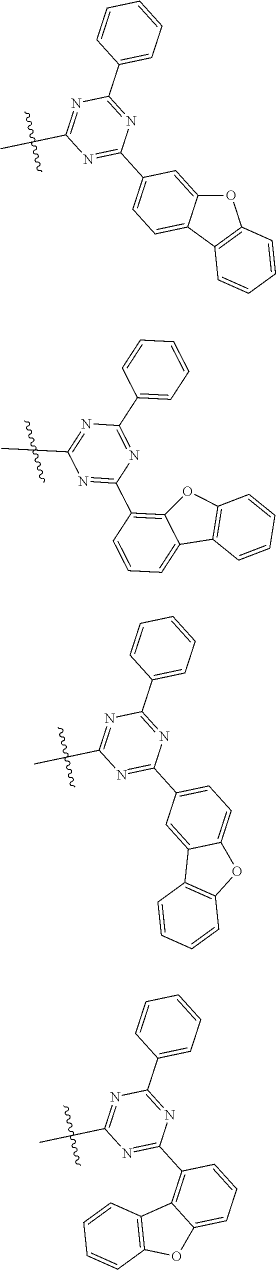

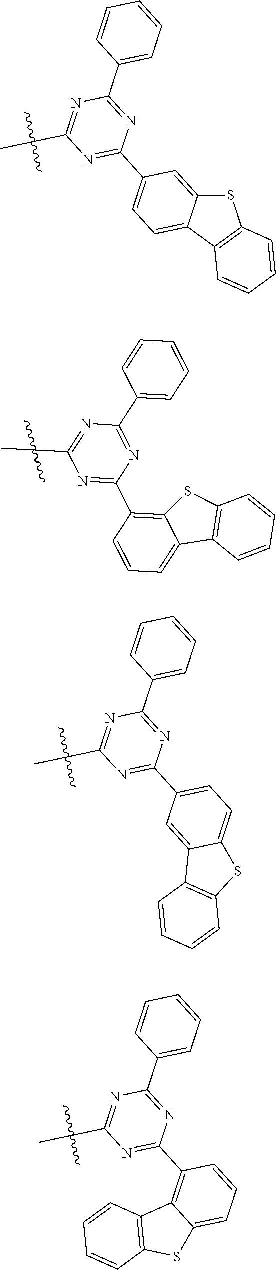

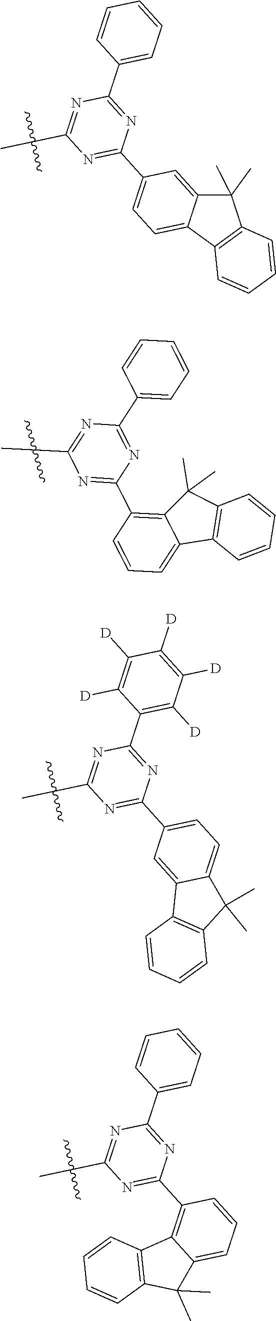

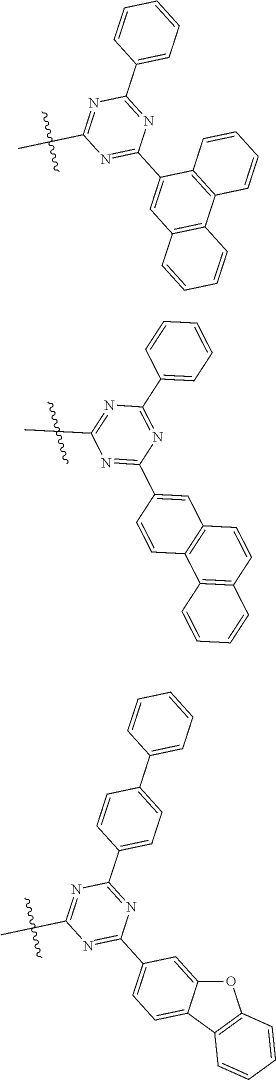

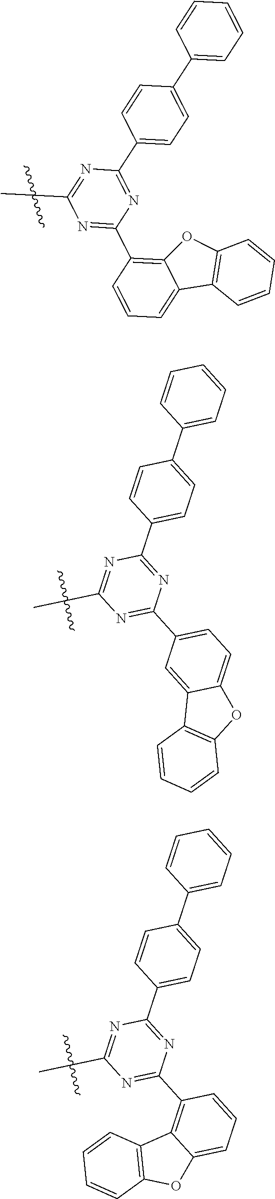

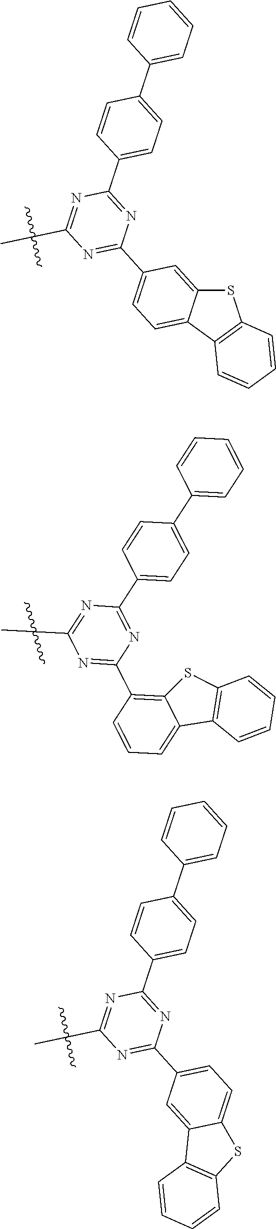 [SMILES: *c1nc(-c2ccc(-c3ccccc3)cc2)nc(-c2ccc3c(c2)sc2ccccc23)n1 ; *c1nc(-c2ccc(-c3ccccc3)cc2)nc(-c2cccc3c2sc2ccccc23)n1 ; *c1nc(-c2ccc(-c3ccccc3)cc2)nc(-c2ccc3sc4ccccc4c3c2)n1]

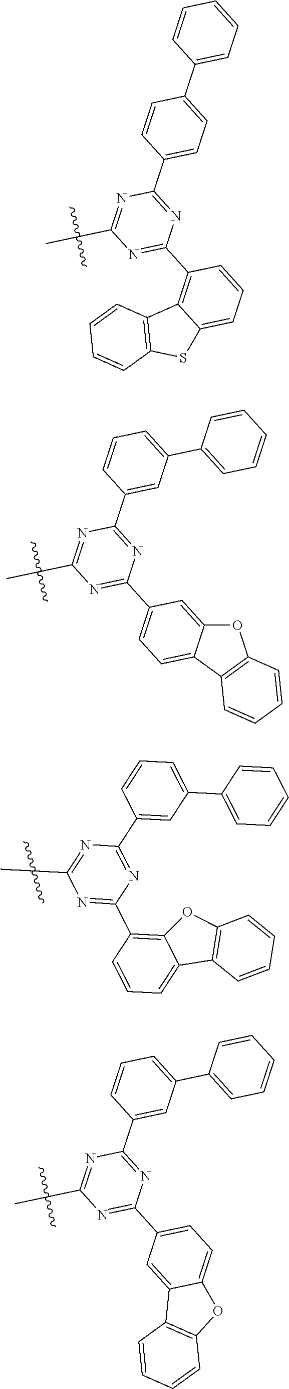

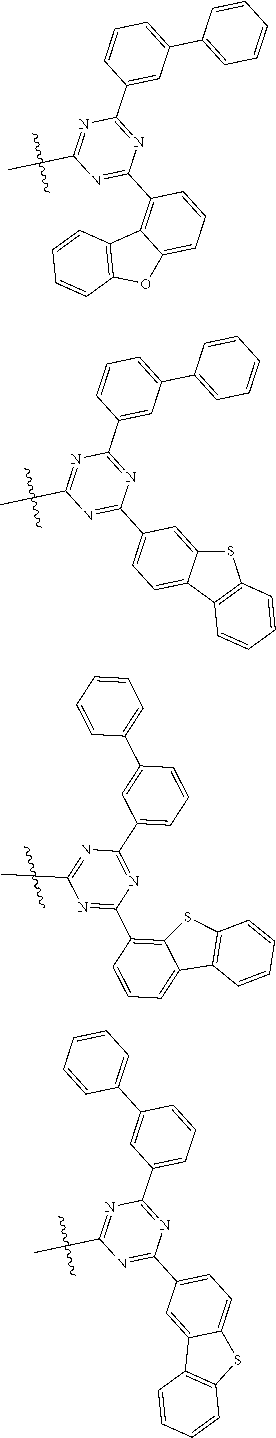

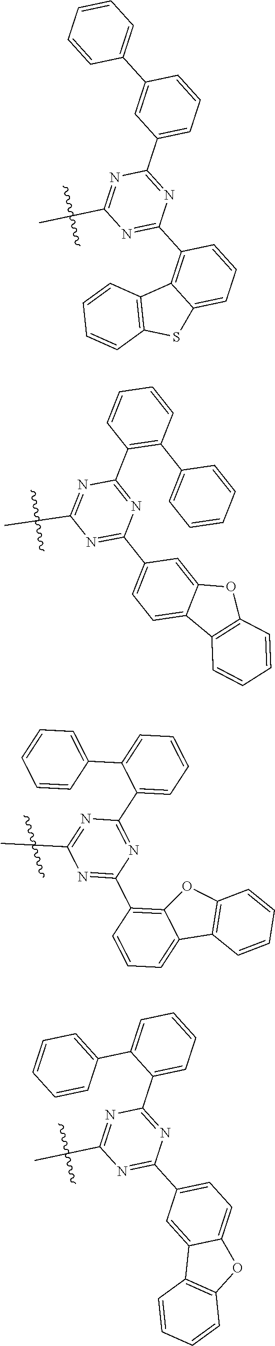

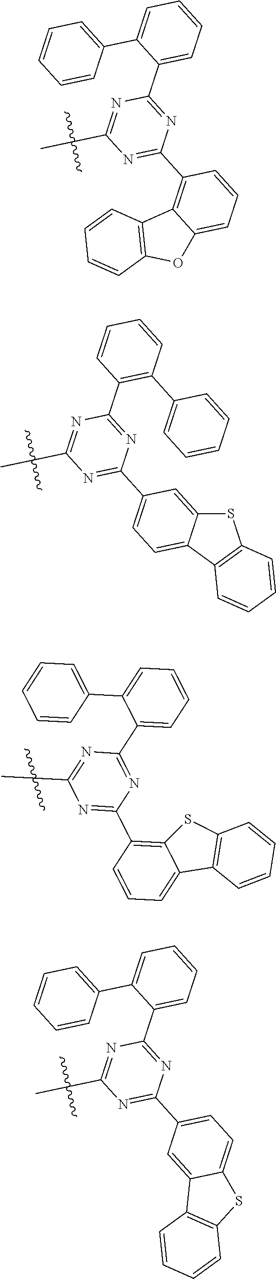 [SMILES: *c1nc(-c2ccccc2-c2ccccc2)nc(-c2cccc3oc4ccccc4c23)n1 ; *c1nc(-c2ccc3c(c2)sc2ccccc23)nc(-c2ccccc2-c2ccccc2)n1 ; *c1nc(-c2ccccc2-c2ccccc2)nc(-c2cccc3c2sc2ccccc23)n1 ; *c1nc(-c2ccc3sc4ccccc4c3c2)nc(-c2ccccc2-c2ccccc2)n1]

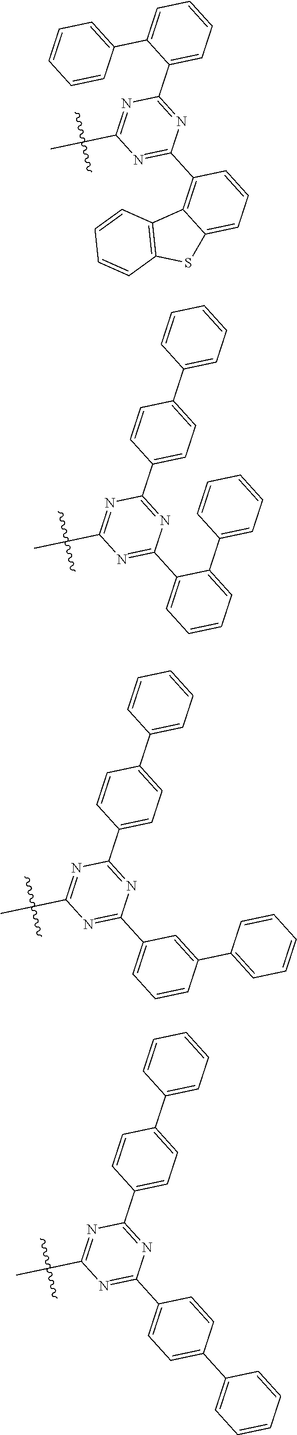

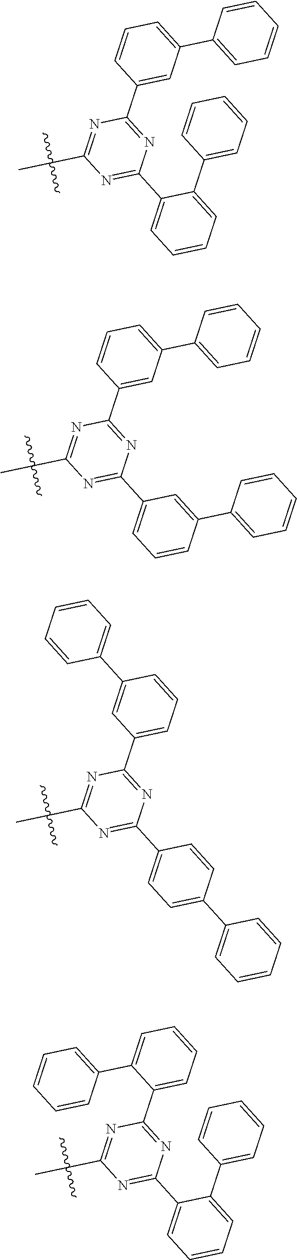 [SMILES: *c1nc(-c2cccc(-c3ccccc3)c2)nc(-c2ccccc2-c2ccccc2)n1 ; *c1nc(-c2cccc(-c3ccccc3)c2)nc(-c2cccc(-c3ccccc3)c2)n1 ; *c1nc(-c2ccc(-c3ccccc3)cc2)nc(-c2cccc(-c3ccccc3)c2)n1 ; *c1nc(-c2ccccc2-c2ccccc2)nc(-c2ccccc2-c2ccccc2)n1]

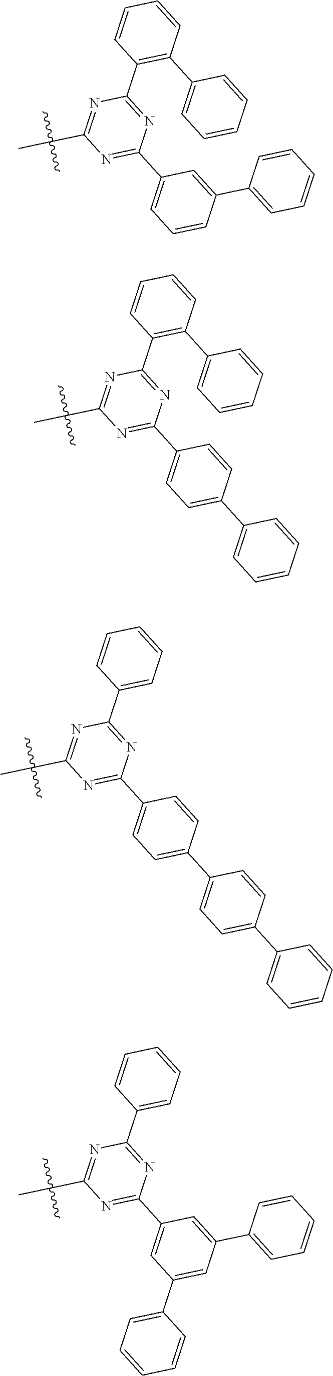

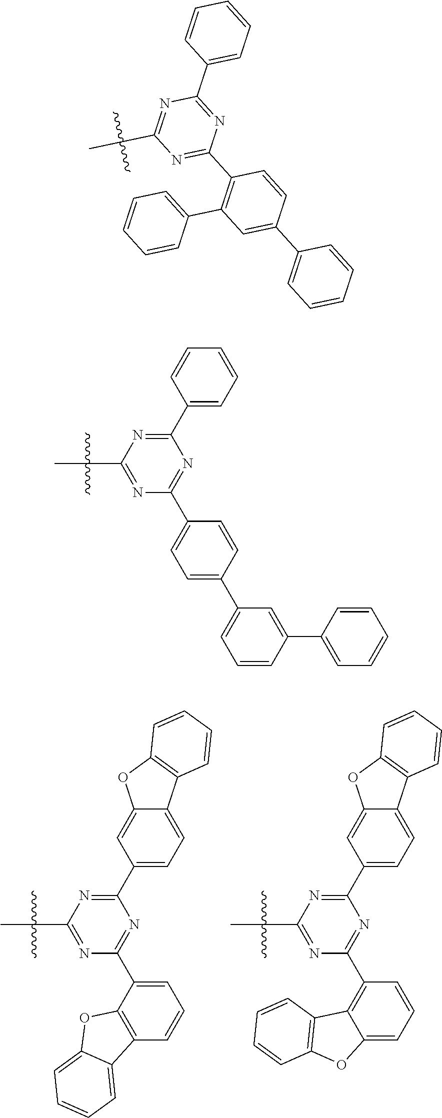 [SMILES: *c1nc(-c2ccccc2)nc(-c2ccc(-c3ccccc3)cc2-c2ccccc2)n1 ; *c1nc(-c2ccccc2)nc(-c2ccc(-c3cccc(-c4ccccc4)c3)cc2)n1 ; *c1nc(-c2ccc3c(c2)oc2ccccc23)nc(-c2cccc3c2oc2ccccc23)n1 ; *c1nc(-c2ccc3c(c2)oc2ccccc23)nc(-c2cccc3oc4ccccc4c23)n1]

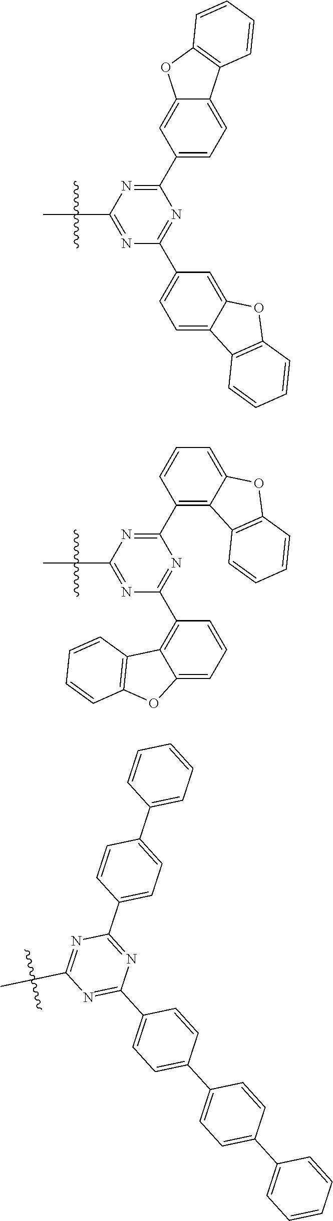

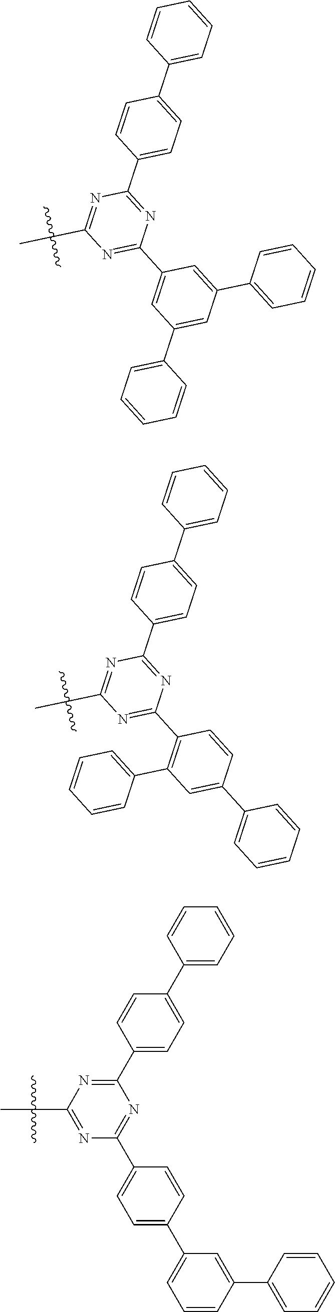 [SMILES: *c1nc(-c2ccc(-c3ccccc3)cc2)nc(-c2cc(-c3ccccc3)cc(-c3ccccc3)c2)n1 ; *c1nc(-c2ccc(-c3ccccc3)cc2)nc(-c2ccc(-c3ccccc3)cc2-c2ccccc2)n1 ; *c1nc(-c2ccc(-c3ccccc3)cc2)nc(-c2ccc(-c3cccc(-c4ccccc4)c3)cc2)n1]

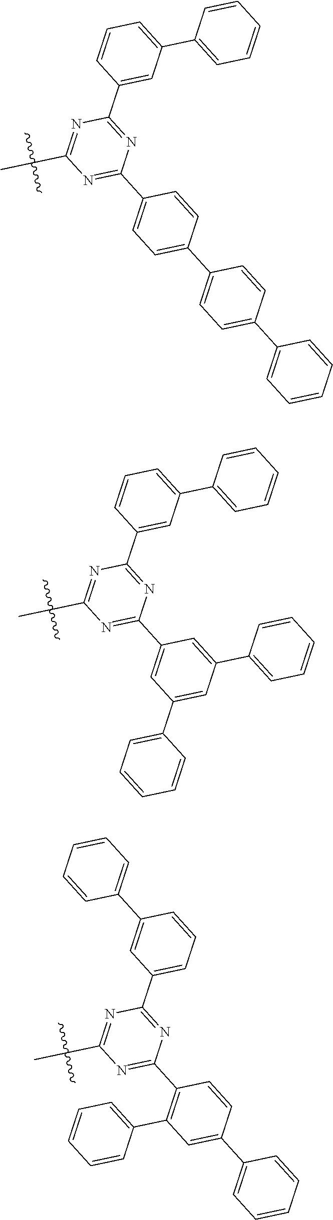 [SMILES: *c1nc(-c2ccc(-c3ccc(-c4ccccc4)cc3)cc2)nc(-c2cccc(-c3ccccc3)c2)n1 ; *c1nc(-c2cccc(-c3ccccc3)c2)nc(-c2cc(-c3ccccc3)cc(-c3ccccc3)c2)n1 ; *c1nc(-c2cccc(-c3ccccc3)c2)nc(-c2ccc(-c3ccccc3)cc2-c2ccccc2)n1]

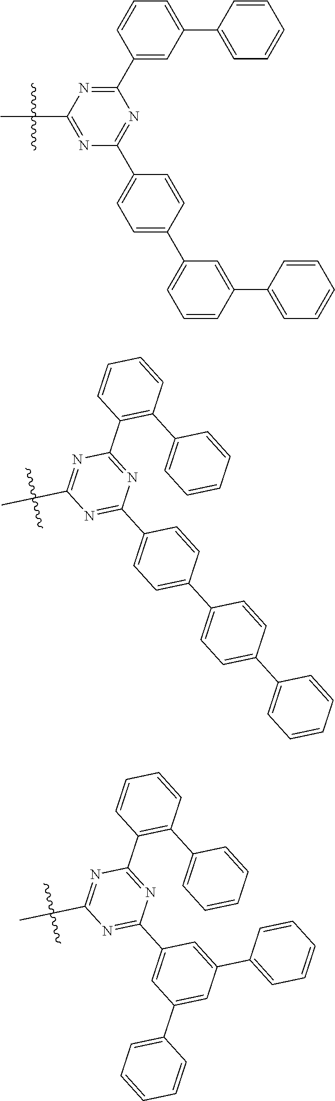 [SMILES: *c1nc(-c2ccc(-c3cccc(-c4ccccc4)c3)cc2)nc(-c2cccc(-c3ccccc3)c2)n1 ; *c1nc(-c2ccc(-c3ccc(-c4ccccc4)cc3)cc2)nc(-c2ccccc2-c2ccccc2)n1 ; *c1nc(-c2cc(-c3ccccc3)cc(-c3ccccc3)c2)nc(-c2ccccc2-c2ccccc2)n1]

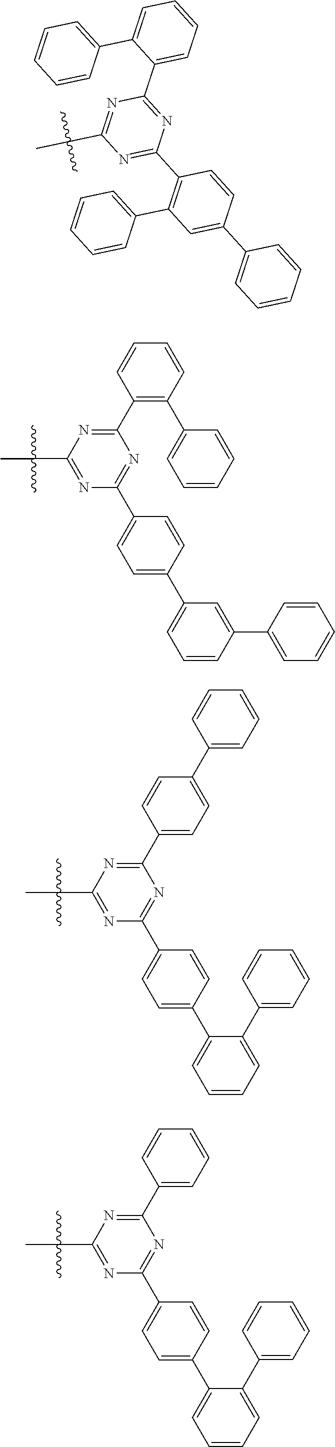

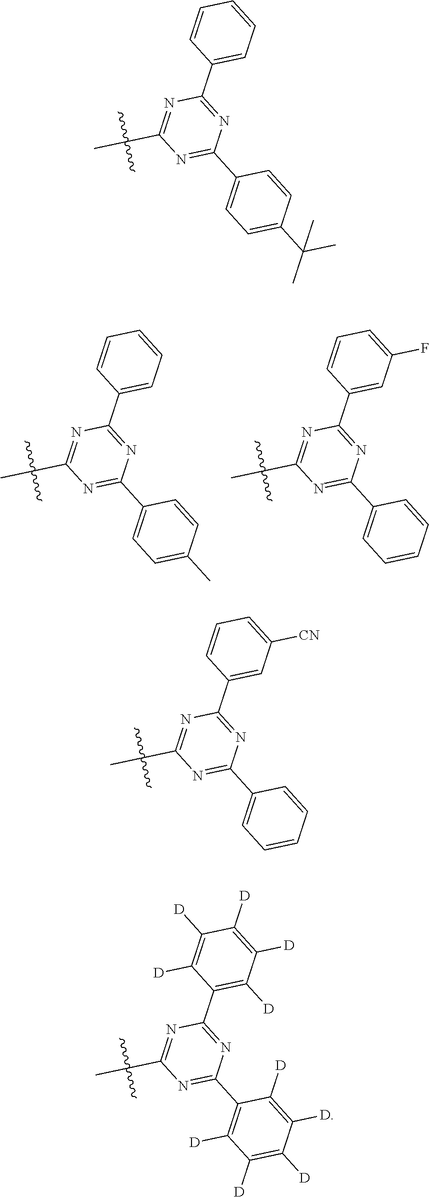

In some implementations of the present disclosure, the organic compound is selected from a group consisting of the following compounds:

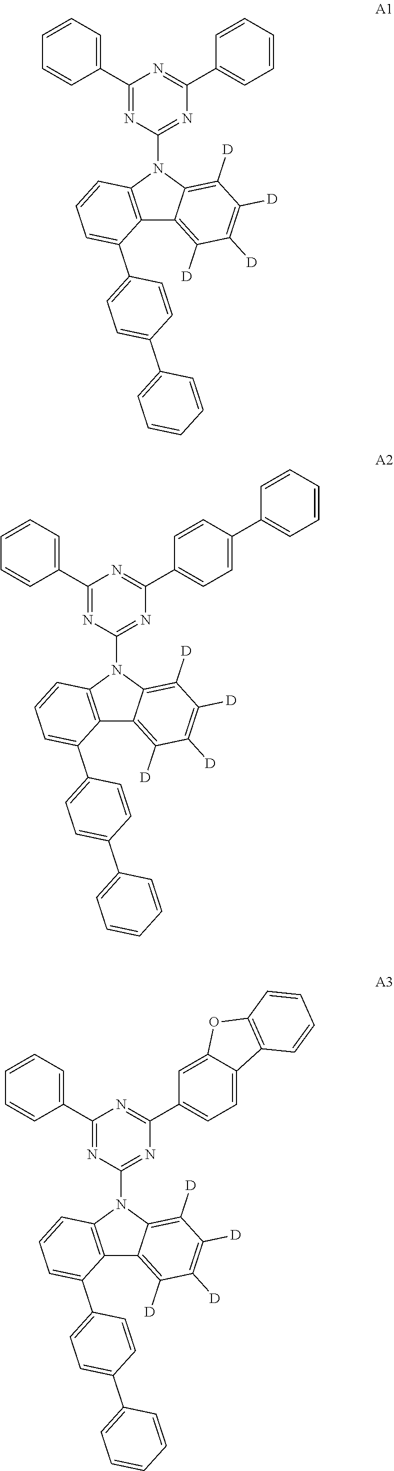 [SMILES: [2H]c1c([2H])c([2H])c2c(c1[2H])c1c(-c3ccc(-c4ccccc4)cc3)cccc1n2-c1nc(-c2ccccc2)nc(-c2ccccc2)n1 ; [2H]c1c([2H])c([2H])c2c(c1[2H])c1c(-c3ccc(-c4ccccc4)cc3)cccc1n2-c1nc(-c2ccccc2)nc(-c2ccc(-c3ccccc3)cc2)n1 ; [2H]c1c([2H])c([2H])c2c(c1[2H])c1c(-c3ccc(-c4ccccc4)cc3)cccc1n2-c1nc(-c2ccccc2)nc(-c2ccc3c(c2)oc2ccccc23)n1]

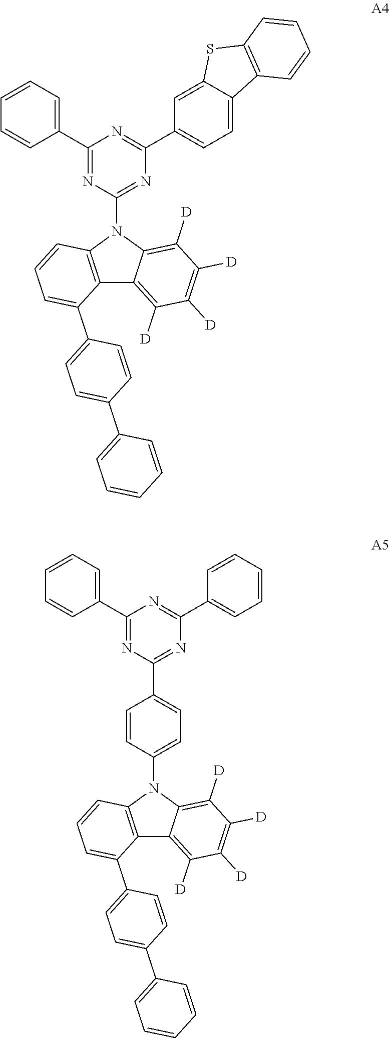 [SMILES: [2H]c1c([2H])c([2H])c2c(c1[2H])c1c(-c3ccc(-c4ccccc4)cc3)cccc1n2-c1nc(-c2ccccc2)nc(-c2ccc3c(c2)sc2ccccc23)n1 ; [2H]c1c([2H])c([2H])c2c(c1[2H])c1c(-c3ccc(-c4ccccc4)cc3)cccc1n2-c1ccc(-c2nc(-c3ccccc3)nc(-c3ccccc3)n2)cc1]

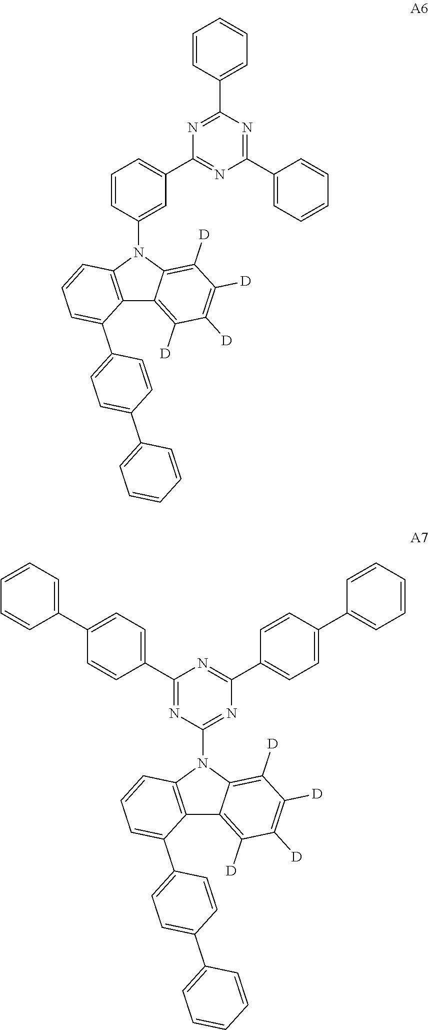 [SMILES: [2H]c1c([2H])c([2H])c2c(c1[2H])c1c(-c3ccc(-c4ccccc4)cc3)cccc1n2-c1cccc(-c2nc(-c3ccccc3)nc(-c3ccccc3)n2)c1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc(-c3ccccc3)cc2)n1]

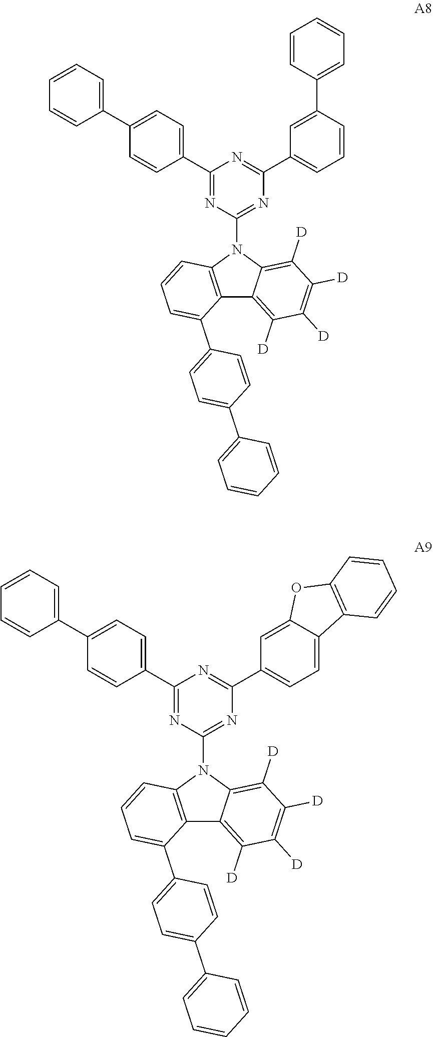 [SMILES: [2H]c1c([2H])c([2H])c2c(c1[2H])c1c(-c3ccc(-c4ccccc4)cc3)cccc1n2-c1nc(-c2ccc(-c3ccccc3)cc2)nc(-c2cccc(-c3ccccc3)c2)n1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3c(c2)oc2ccccc23)n1]

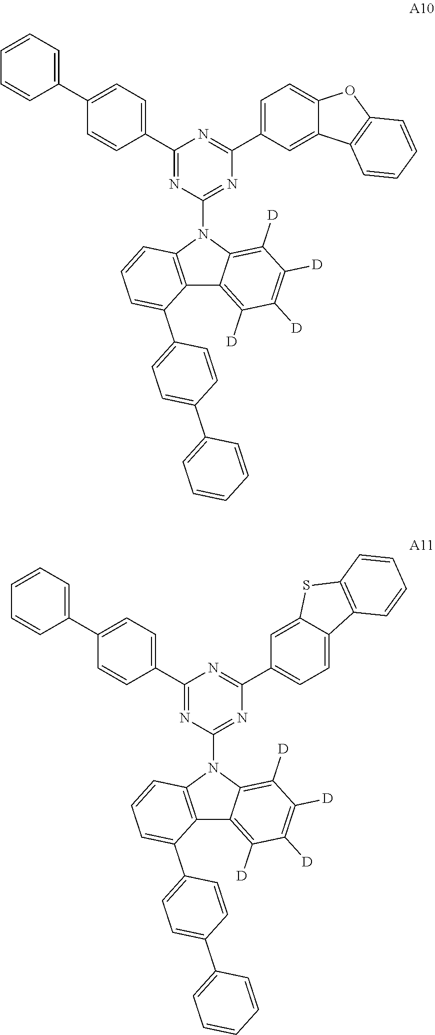 [SMILES: [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3oc4ccccc4c3c2)n1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3c(c2)sc2ccccc23)n1]

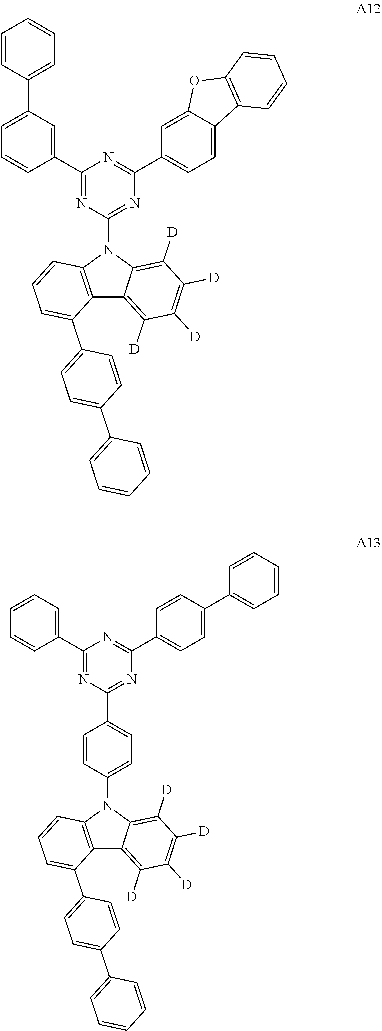 [SMILES: [2H]c1c([2H])c([2H])c2c(c1[2H])c1c(-c3ccc(-c4ccccc4)cc3)cccc1n2-c1nc(-c2cccc(-c3ccccc3)c2)nc(-c2ccc3c(c2)oc2ccccc23)n1 ; [2H]c1c([2H])c([2H])c2c(c1[2H])c1c(-c3ccc(-c4ccccc4)cc3)cccc1n2-c1ccc(-c2nc(-c3ccccc3)nc(-c3ccc(-c4ccccc4)cc3)n2)cc1]

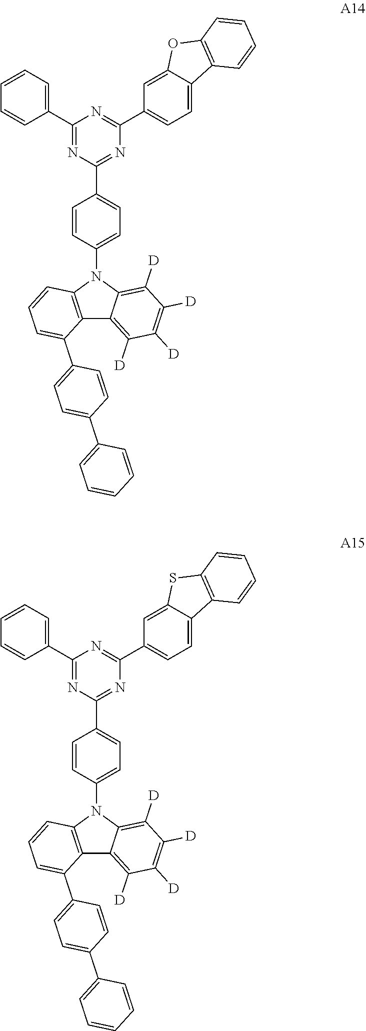 [SMILES: [2H]c1c([2H])c([2H])c2c(c1[2H])c1c(-c3ccc(-c4ccccc4)cc3)cccc1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3ccc(-c4ccccc4)cc3)cccc1n2-c1ccc(-c2nc(-c3ccccc3)nc(-c3ccc4c(c3)sc3ccccc34)n2)cc1]

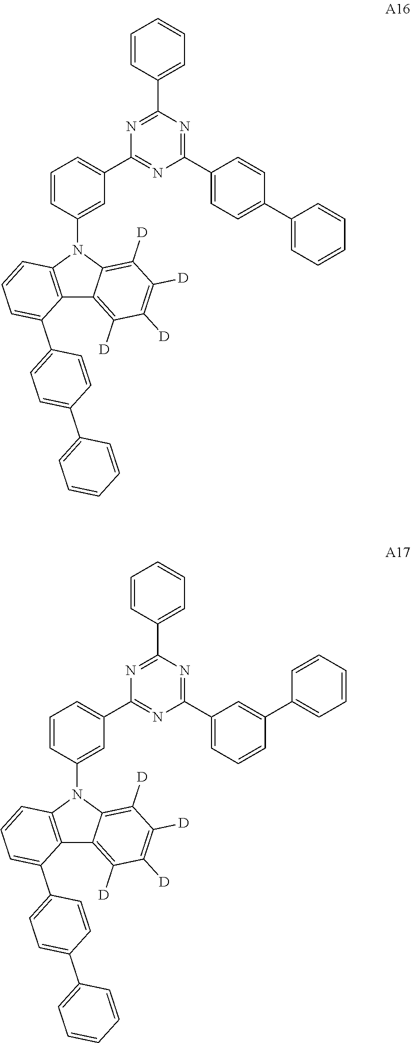 [SMILES: [2H]c1c([2H])c([2H])c2c(c1[2H])c1c(-c3ccc(-c4ccccc4)cc3)cccc1n2-c1cccc(-c2nc(-c3ccccc3)nc(-c3ccc(-c4ccccc4)cc3)n2)c1 ; [2H]c1c([2H])c([2H])c2c(c1[2H])c1c(-c3ccc(-c4ccccc4)cc3)cccc1n2-c1cccc(-c2nc(-c3ccccc3)nc(-c3cccc(-c4ccccc4)c3)n2)c1]

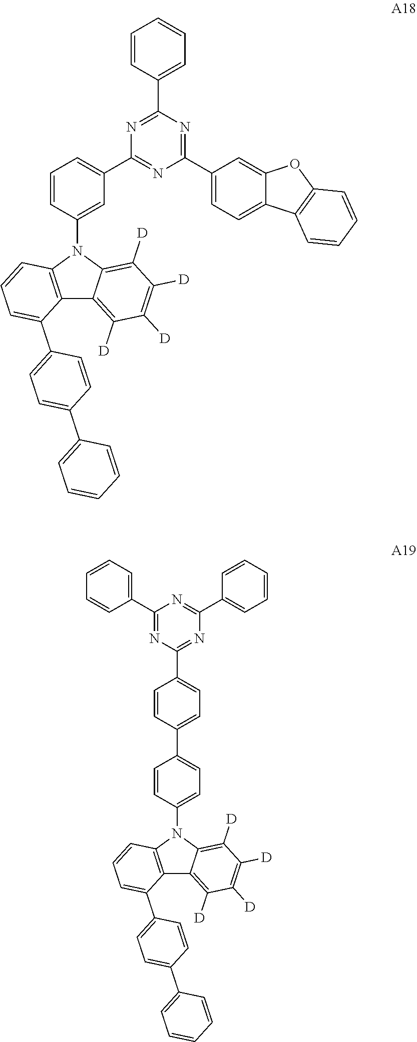 [SMILES: [2H]c1c([2H])c([2H])c2c(c1[2H])c1c(-c3ccc(-c4ccccc4)cc3)cccc1n2-c1cccc(-c2nc(-c3ccccc3)nc(-c3ccc4c(c3)oc3ccccc34)n2)c1 ; [2H]c1c([2H])c([2H])c2c(c1[2H])c1c(-c3ccc(-c4ccccc4)cc3)cccc1n2-c1ccc(-c2ccc(-c3nc(-c4ccccc4)nc(-c4ccccc4)n3)cc2)cc1]

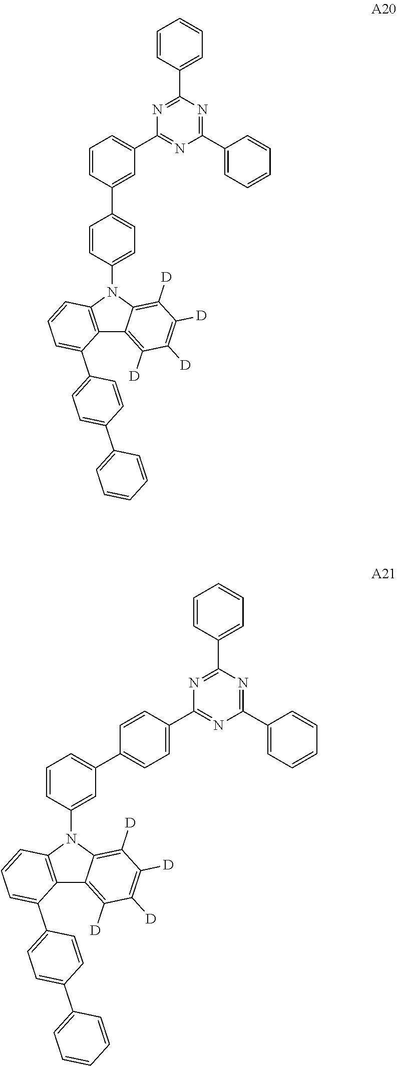 [SMILES: [2H]c1c([2H])c([2H])c2c(c1[2H])c1c(-c3ccc(-c4ccccc4)cc3)cccc1n2-c1ccc(-c2cccc(-c3nc(-c4ccccc4)nc(-c4ccccc4)n3)c2)cc1 ; [2H]c1c([2H])c([2H])c2c(c1[2H])c1c(-c3ccc(-c4ccccc4)cc3)cccc1n2-c1cccc(-c2ccc(-c3nc(-c4ccccc4)nc(-c4ccccc4)n3)cc2)c1]

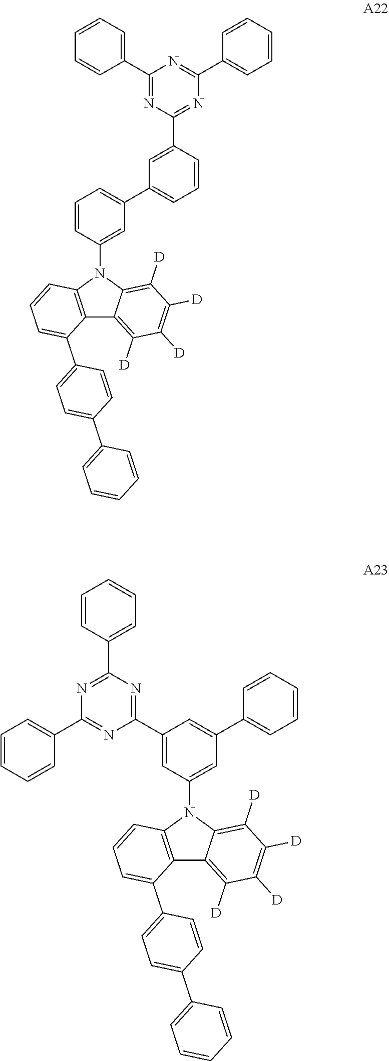 [SMILES: [2H]c1c([2H])c([2H])c2c(c1[2H])c1c(-c3ccc(-c4ccccc4)cc3)cccc1n2-c1cccc(-c2cccc(-c3nc(-c4ccccc4)nc(-c4ccccc4)n3)c2)c1 ; [2H]c1c([2H])c([2H])c2c(c1[2H])c1c(-c3ccc(-c4ccccc4)cc3)cccc1n2-c1cc(-c2ccccc2)cc(-c2nc(-c3ccccc3)nc(-c3ccccc3)n2)c1]

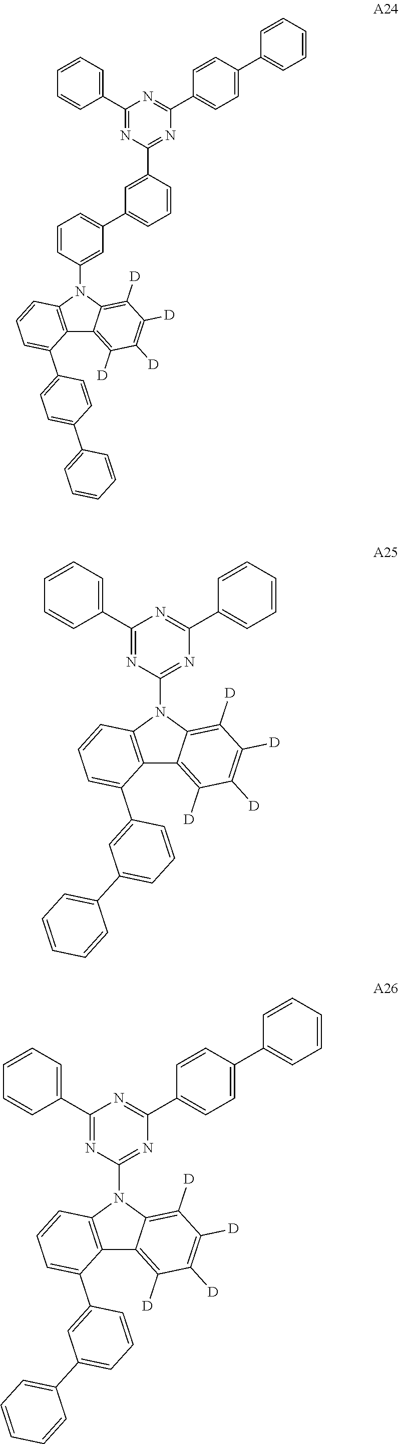 [SMILES: [2H]c1c([2H])c([2H])c2c(c1[2H])c1c(-c3ccc(-c4ccccc4)cc3)cccc1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1c(-c3cccc(-c4ccccc4)c3)cccc1n2-c1nc(-c2ccccc2)nc(-c2ccccc2)n1 ; [2H]c1c([2H])c([2H])c2c(c1[2H])c1c(-c3cccc(-c4ccccc4)c3)cccc1n2-c1nc(-c2ccccc2)nc(-c2ccc(-c3ccccc3)cc2)n1]

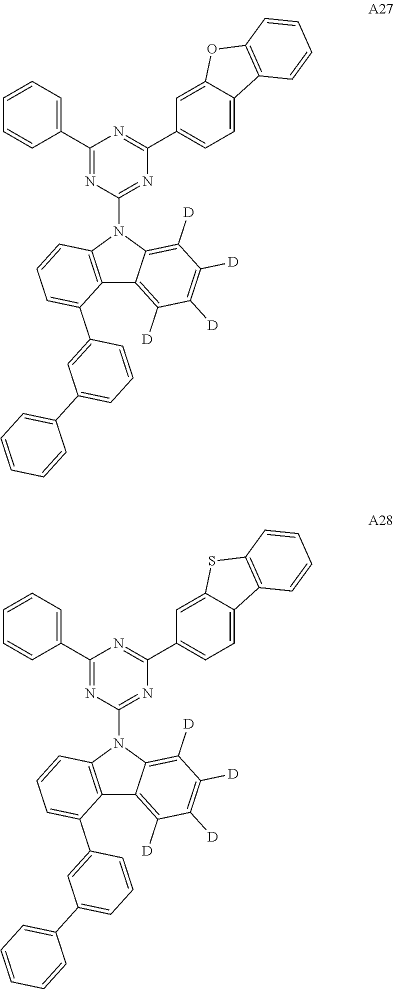 [SMILES: [2H]c1c([2H])c([2H])c2c(c1[2H])c1c(-c3cccc(-c4ccccc4)c3)cccc1n2-c1nc(-c2ccccc2)nc(-c2ccc3c(c2)oc2ccccc23)n1 ; [2H]c1c([2H])c([2H])c2c(c1[2H])c1c(-c3cccc(-c4ccccc4)c3)cccc1n2-c1nc(-c2ccccc2)nc(-c2ccc3c(c2)sc2ccccc23)n1]

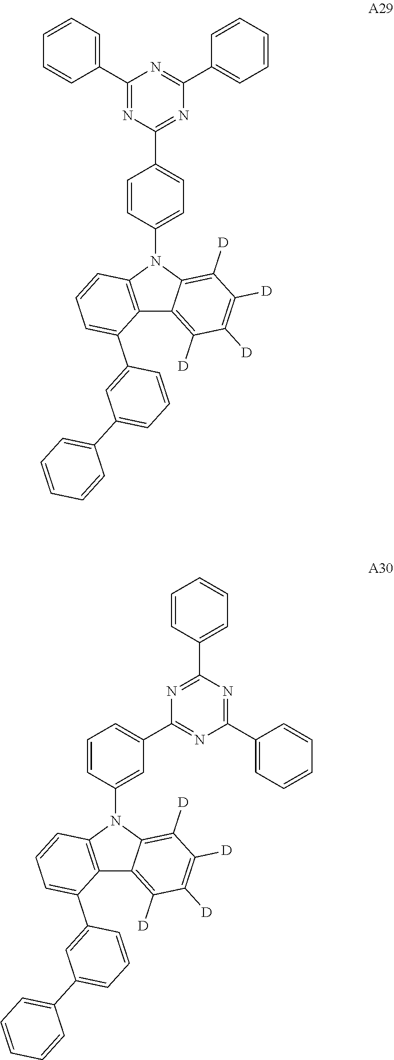 [SMILES: [2H]c1c([2H])c([2H])c2c(c1[2H])c1c(-c3cccc(-c4ccccc4)c3)cccc1n2-c1ccc(-c2nc(-c3ccccc3)nc(-c3ccccc3)n2)cc1 ; [2H]c1c([2H])c([2H])c2c(c1[2H])c1c(-c3cccc(-c4ccccc4)c3)cccc1n2-c1cccc(-c2nc(-c3ccccc3)nc(-c3ccccc3)n2)c1]

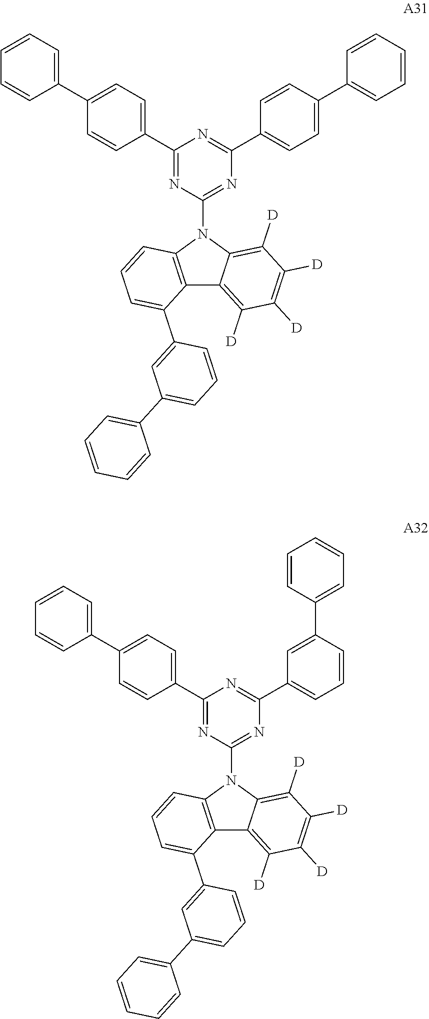 [SMILES: [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc(-c3ccccc3)cc2)n1 ; [2H]c1c([2H])c([2H])c2c(c1[2H])c1c(-c3cccc(-c4ccccc4)c3)cccc1n2-c1nc(-c2ccc(-c3ccccc3)cc2)nc(-c2cccc(-c3ccccc3)c2)n1]

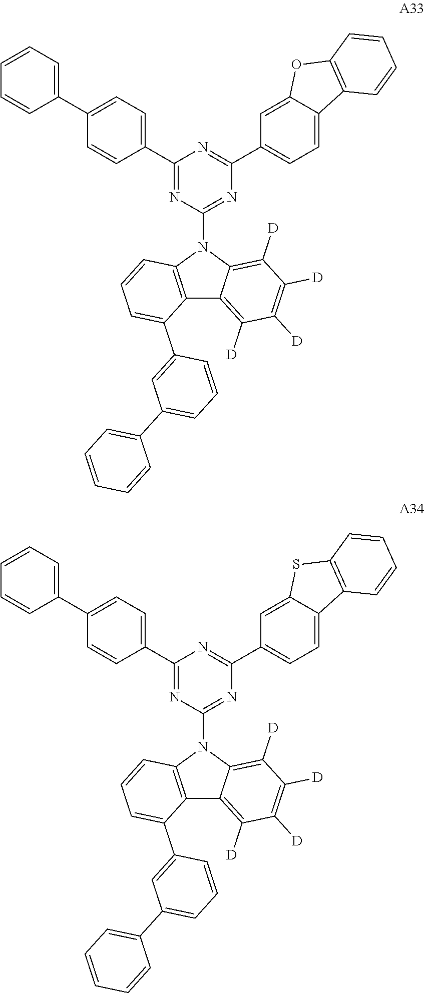 [SMILES: [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc3c(c2)oc2ccccc23)n1 ; [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc3c(c2)sc2ccccc23)n1]

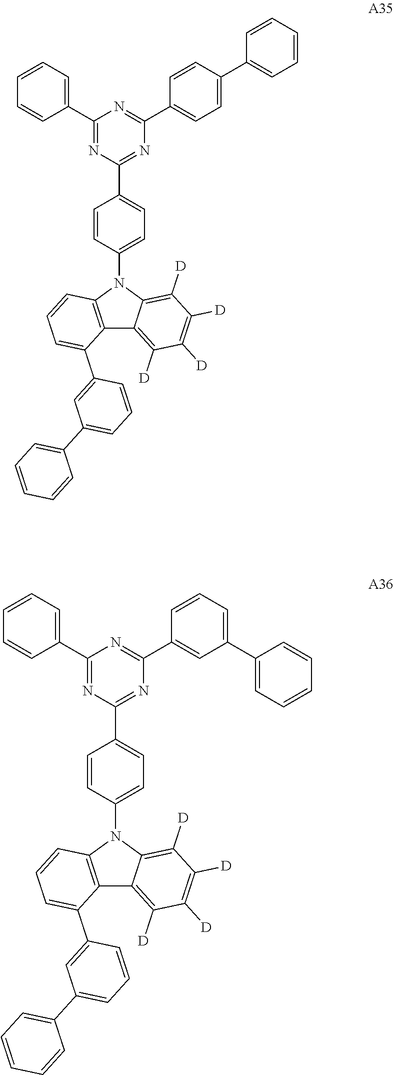 [SMILES: [2H]c1c([2H])c([2H])c2c(c1[2H])c1c(-c3cccc(-c4ccccc4)c3)cccc1n2-c1ccc(-c2nc(-c3ccccc3)nc(-c3ccc(-c4ccccc4)cc3)n2)cc1 ; [2H]c1c([2H])c([2H])c2c(c1[2H])c1c(-c3cccc(-c4ccccc4)c3)cccc1n2-c1ccc(-c2nc(-c3ccccc3)nc(-c3cccc(-c4ccccc4)c3)n2)cc1]

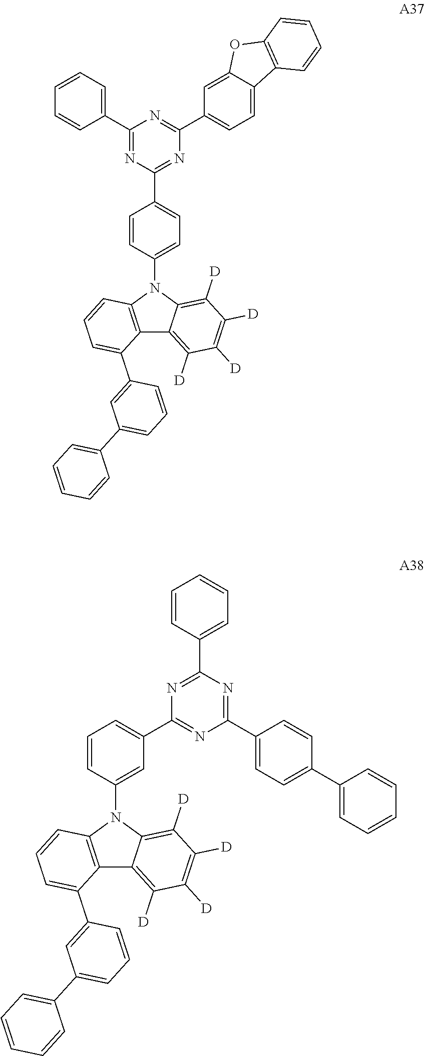 [SMILES: [2H]c1c([2H])c([2H])c2c(c1[2H])c1c(-c3cccc(-c4ccccc4)c3)cccc1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3cccc(-c4ccccc4)c3)cccc1n2-c1cccc(-c2nc(-c3ccccc3)nc(-c3ccc(-c4ccccc4)cc3)n2)c1]

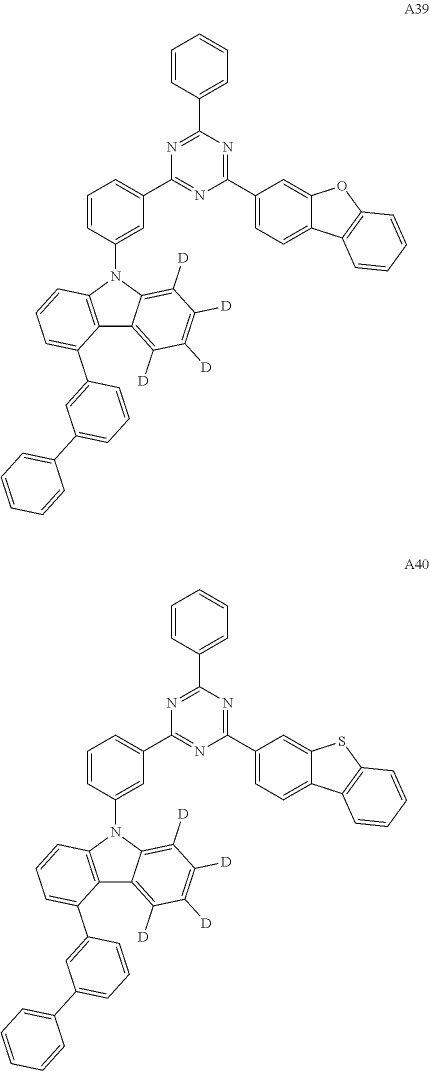 [SMILES: [2H]c1c([2H])c([2H])c2c(c1[2H])c1c(-c3cccc(-c4ccccc4)c3)cccc1n2-c1cccc(-c2nc(-c3ccccc3)nc(-c3ccc4c(c3)oc3ccccc34)n2)c1 ; [2H]c1c([2H])c([2H])c2c(c1[2H])c1c(-c3cccc(-c4ccccc4)c3)cccc1n2-c1cccc(-c2nc(-c3ccccc3)nc(-c3ccc4c(c3)sc3ccccc34)n2)c1]

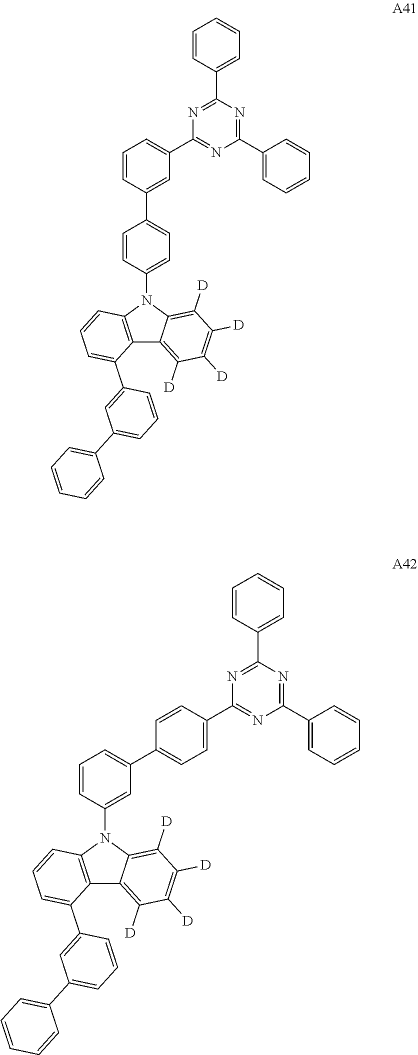 [SMILES: [2H]c1c([2H])c([2H])c2c(c1[2H])c1c(-c3cccc(-c4ccccc4)c3)cccc1n2-c1ccc(-c2cccc(-c3nc(-c4ccccc4)nc(-c4ccccc4)n3)c2)cc1 ; [2H]c1c([2H])c([2H])c2c(c1[2H])c1c(-c3cccc(-c4ccccc4)c3)cccc1n2-c1cccc(-c2ccc(-c3nc(-c4ccccc4)nc(-c4ccccc4)n3)cc2)c1]

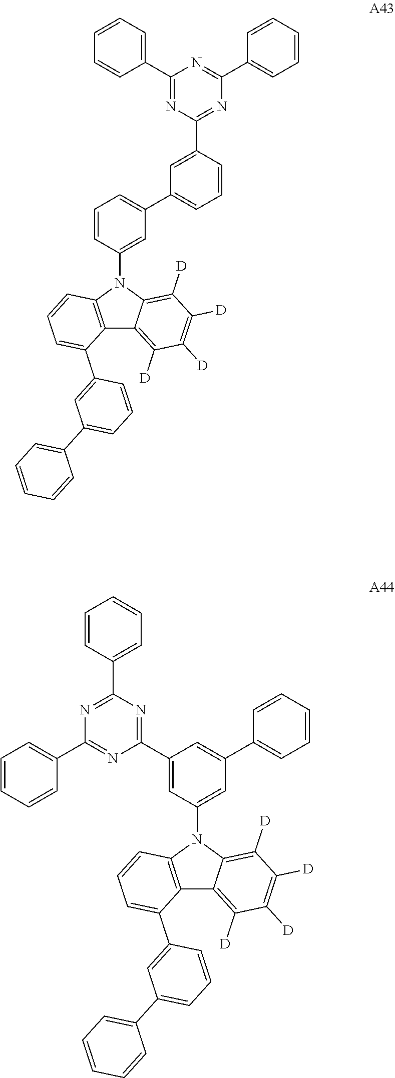 [SMILES: [2H]c1c([2H])c([2H])c2c(c1[2H])c1c(-c3cccc(-c4ccccc4)c3)cccc1n2-c1cccc(-c2cccc(-c3nc(-c4ccccc4)nc(-c4ccccc4)n3)c2)c1 ; [2H]c1c([2H])c([2H])c2c(c1[2H])c1c(-c3cccc(-c4ccccc4)c3)cccc1n2-c1cc(-c2ccccc2)cc(-c2nc(-c3ccccc3)nc(-c3ccccc3)n2)c1]

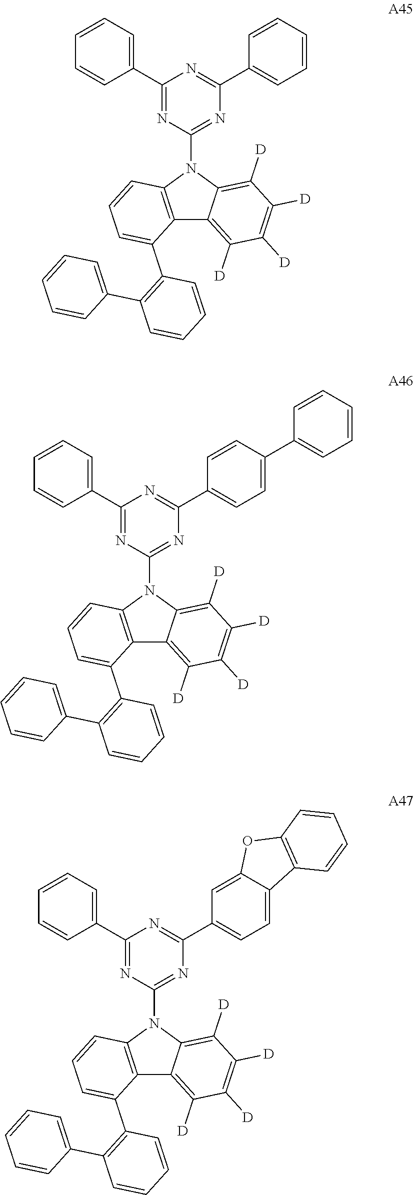 [SMILES: [2H]c1c([2H])c([2H])c2c(c1[2H])c1c(-c3ccccc3-c3ccccc3)cccc1n2-c1nc(-c2ccccc2)nc(-c2ccccc2)n1 ; [2H]c1c([2H])c([2H])c2c(c1[2H])c1c(-c3ccccc3-c3ccccc3)cccc1n2-c1nc(-c2ccccc2)nc(-c2ccc(-c3ccccc3)cc2)n1 ; [2H]c1c([2H])c([2H])c2c(c1[2H])c1c(-c3ccccc3-c3ccccc3)cccc1n2-c1nc(-c2ccccc2)nc(-c2ccc3c(c2)oc2ccccc23)n1]

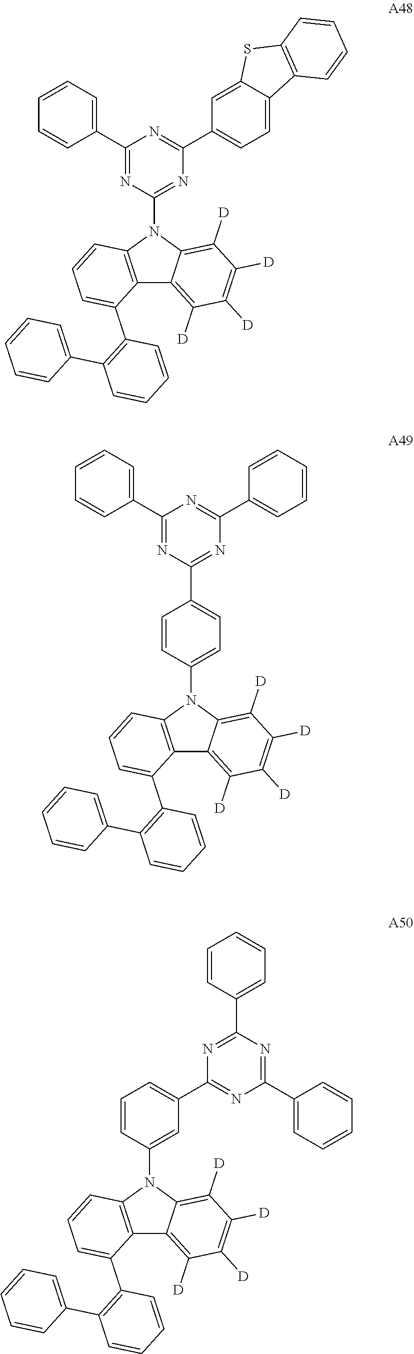 [SMILES: [2H]c1c([2H])c([2H])c2c(c1[2H])c1c(-c3ccccc3-c3ccccc3)cccc1n2-c1nc(-c2ccccc2)nc(-c2ccc3c(c2)sc2ccccc23)n1 ; [2H]c1c([2H])c([2H])c2c(c1[2H])c1c(-c3ccccc3-c3ccccc3)cccc1n2-c1ccc(-c2nc(-c3ccccc3)nc(-c3ccccc3)n2)cc1 ; [2H]c1c([2H])c([2H])c2c(c1[2H])c1c(-c3ccccc3-c3ccccc3)cccc1n2-c1cccc(-c2nc(-c3ccccc3)nc(-c3ccccc3)n2)c1]

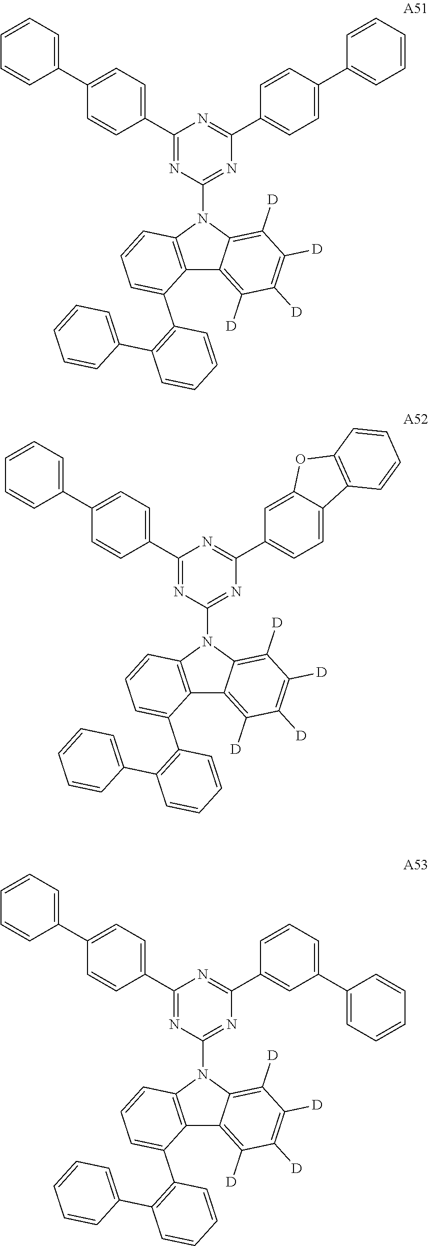 [SMILES: [2H]c1c([2H])c([2H])c2c(c1[2H])c1c(-c3ccccc3-c3ccccc3)cccc1n2-c1nc(-c2ccc(-c3ccccc3)cc2)nc(-c2ccc(-c3ccccc3)cc2)n1 ; [2H]c1c([2H])c([2H])c2c(c1[2H])c1c(-c3ccccc3-c3ccccc3)cccc1n2-c1nc(-c2ccc(-c3ccccc3)cc2)nc(-c2ccc3c(c2)oc2ccccc23)n1 ; [2H]c1c([2H])c([2H])c2c(c1[2H])c1c(-c3ccccc3-c3ccccc3)cccc1n2-c1nc(-c2ccc(-c3ccccc3)cc2)nc(-c2cccc(-c3ccccc3)c2)n1]

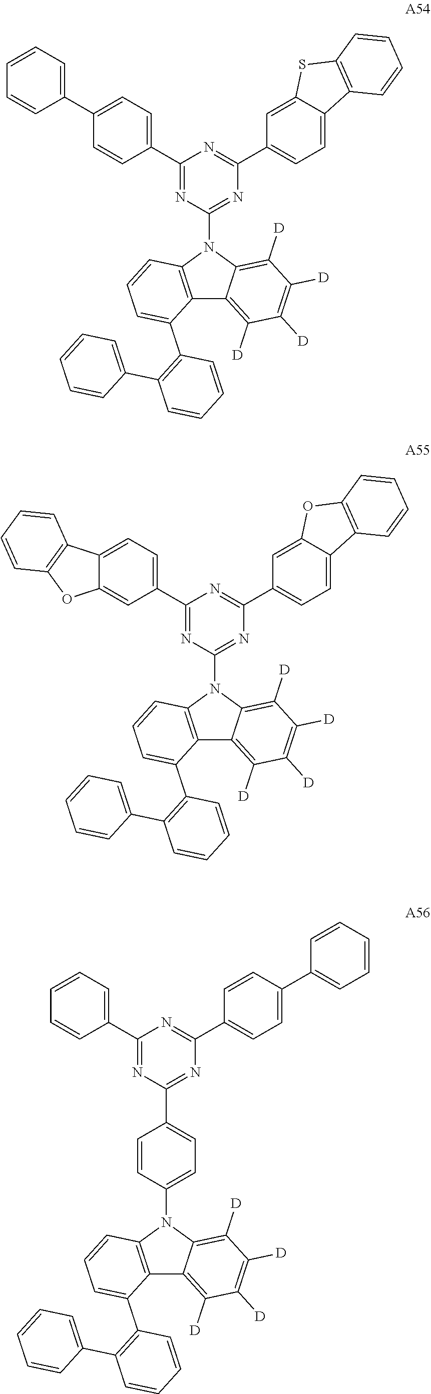 [SMILES: [2H]c1c([2H])c([2H])c2c(c1[2H])c1c(-c3ccccc3-c3ccccc3)cccc1n2-c1nc(-c2ccc(-c3ccccc3)cc2)nc(-c2ccc3c(c2)sc2ccccc23)n1 ; [2H]c1c([2H])c([2H])c2c(c1[2H])c1c(-c3ccccc3-c3ccccc3)cccc1n2-c1nc(-c2ccc3c(c2)oc2ccccc23)nc(-c2ccc3c(c2)oc2ccccc23)n1 ; [2H]c1c([2H])c([2H])c2c(c1[2H])c1c(-c3ccccc3-c3ccccc3)cccc1n2-c1ccc(-c2nc(-c3ccccc3)nc(-c3ccc(-c4ccccc4)cc3)n2)cc1]

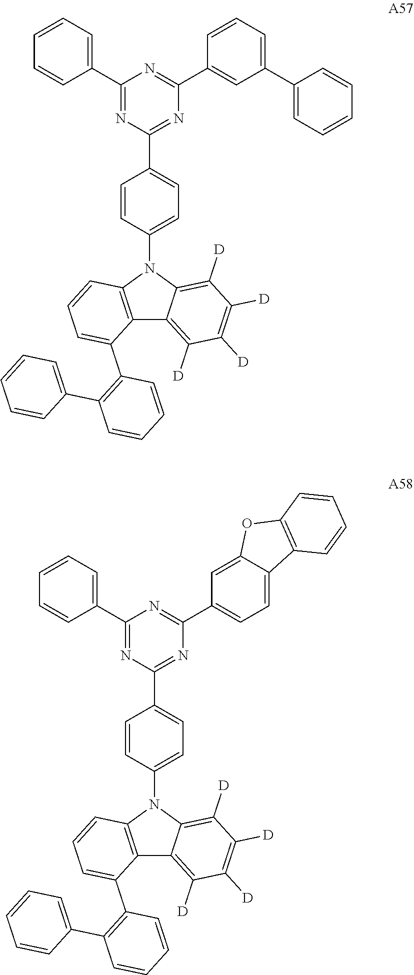 [SMILES: [2H]c1c([2H])c([2H])c2c(c1[2H])c1c(-c3ccccc3-c3ccccc3)cccc1n2-c1ccc(-c2nc(-c3ccccc3)nc(-c3cccc(-c4ccccc4)c3)n2)cc1 ; [2H]c1c([2H])c([2H])c2c(c1[2H])c1c(-c3ccccc3-c3ccccc3)cccc1n2-c1ccc(-c2nc(-c3ccccc3)nc(-c3ccc4c(c3)oc3ccccc34)n2)cc1]

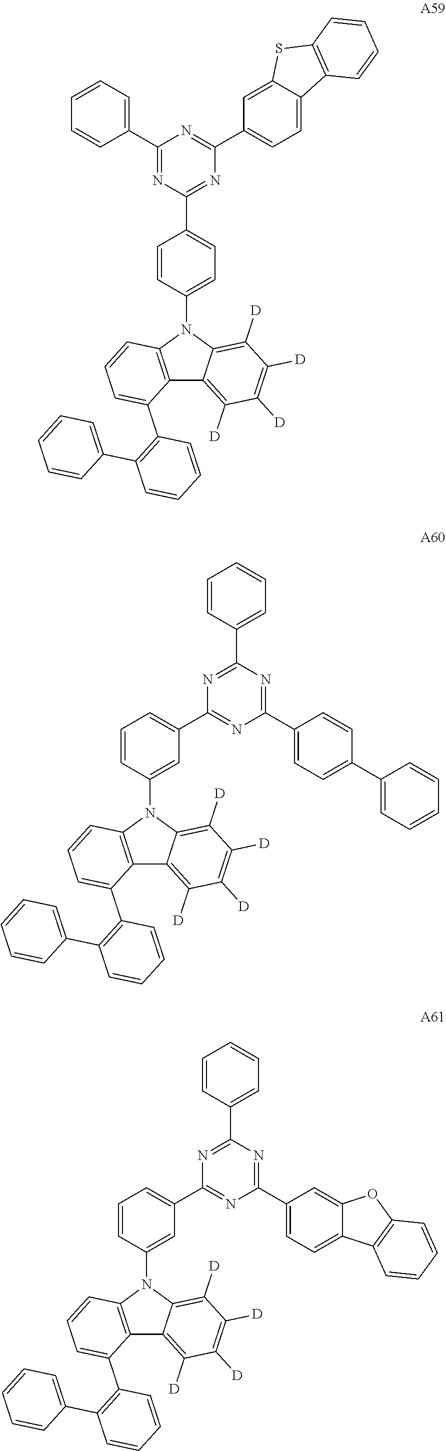 [SMILES: [2H]c1c([2H])c([2H])c2c(c1[2H])c1c(-c3ccccc3-c3ccccc3)cccc1n2-c1ccc(-c2nc(-c3ccccc3)nc(-c3ccc4c(c3)sc3ccccc34)n2)cc1 ; [2H]c1c([2H])c([2H])c2c(c1[2H])c1c(-c3ccccc3-c3ccccc3)cccc1n2-c1cccc(-c2nc(-c3ccccc3)nc(-c3ccc(-c4ccccc4)cc3)n2)c1 ; [2H]c1c([2H])c([2H])c2c(c1[2H])c1c(-c3ccccc3-c3ccccc3)cccc1n2-c1cccc(-c2nc(-c3ccccc3)nc(-c3ccc4c(c3)oc3ccccc34)n2)c1]

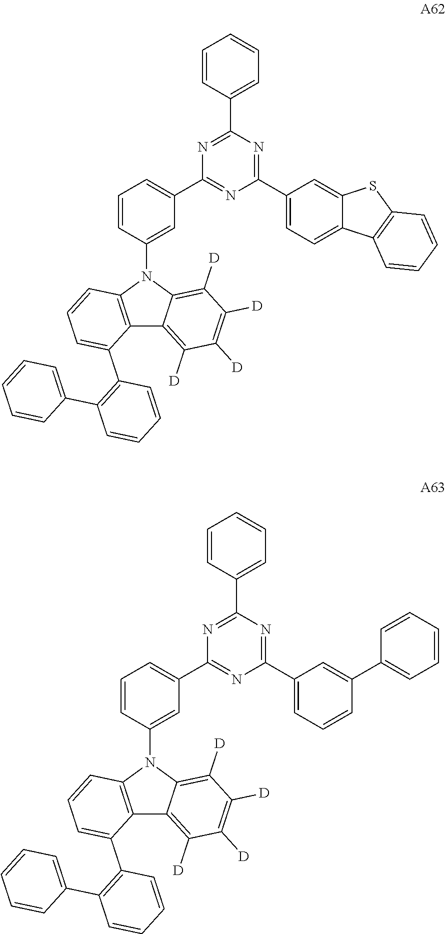 [SMILES: [2H]c1c([2H])c([2H])c2c(c1[2H])c1c(-c3ccccc3-c3ccccc3)cccc1n2-c1cccc(-c2nc(-c3ccccc3)nc(-c3ccc4c(c3)sc3ccccc34)n2)c1 ; [2H]c1c([2H])c([2H])c2c(c1[2H])c1c(-c3ccccc3-c3ccccc3)cccc1n2-c1cccc(-c2nc(-c3ccccc3)nc(-c3cccc(-c4ccccc4)c3)n2)c1]

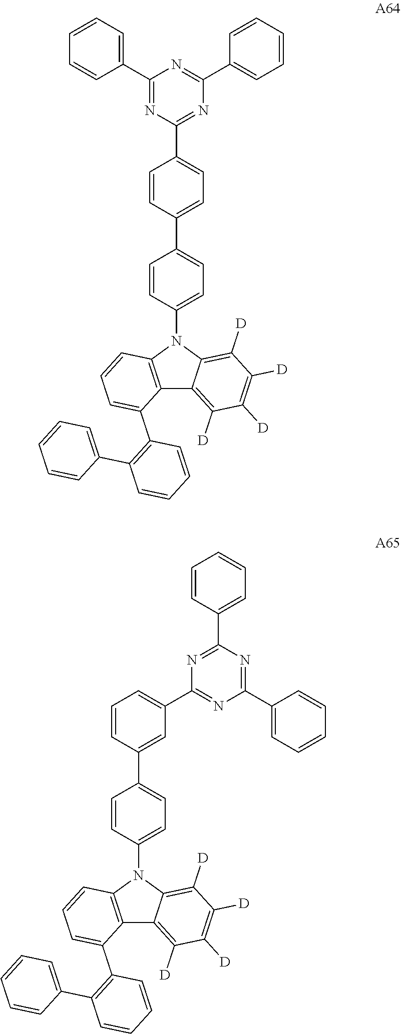 [SMILES: [2H]c1c([2H])c([2H])c2c(c1[2H])c1c(-c3ccccc3-c3ccccc3)cccc1n2-c1ccc(-c2ccc(-c3nc(-c4ccccc4)nc(-c4ccccc4)n3)cc2)cc1 ; [2H]c1c([2H])c([2H])c2c(c1[2H])c1c(-c3ccccc3-c3ccccc3)cccc1n2-c1ccc(-c2cccc(-c3nc(-c4ccccc4)nc(-c4ccccc4)n3)c2)cc1]

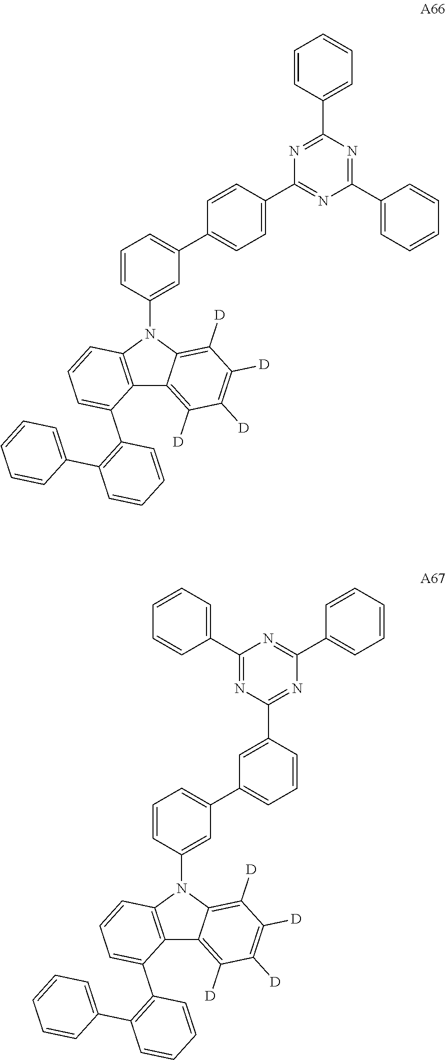 [SMILES: [2H]c1c([2H])c([2H])c2c(c1[2H])c1c(-c3ccccc3-c3ccccc3)cccc1n2-c1cccc(-c2ccc(-c3nc(-c4ccccc4)nc(-c4ccccc4)n3)cc2)c1 ; [2H]c1c([2H])c([2H])c2c(c1[2H])c1c(-c3ccccc3-c3ccccc3)cccc1n2-c1cccc(-c2cccc(-c3nc(-c4ccccc4)nc(-c4ccccc4)n3)c2)c1]

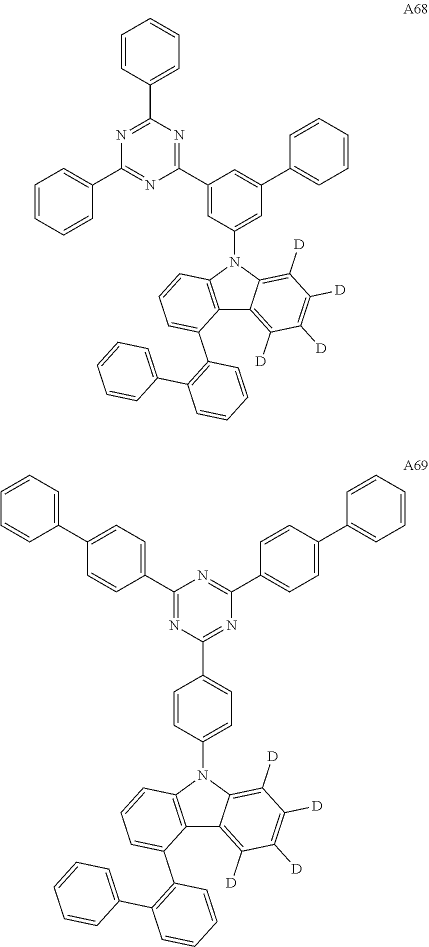 [SMILES: [2H]c1c([2H])c([2H])c2c(c1[2H])c1c(-c3ccccc3-c3ccccc3)cccc1n2-c1cc(-c2ccccc2)cc(-c2nc(-c3ccccc3)nc(-c3ccccc3)n2)c1 ; [2H]c1c([2H])c([2H])c2c(c1[2H])c1c(-c3ccccc3-c3ccccc3)cccc1n2-c1ccc(-c2nc(-c3ccc(-c4ccccc4)cc3)nc(-c3ccc(-c4ccccc4)cc3)n2)cc1]

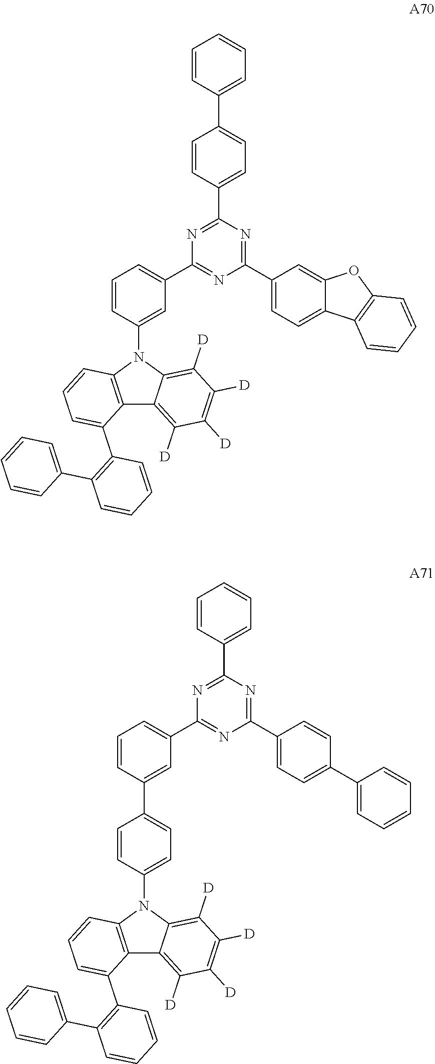 [SMILES: [2H]c1c([2H])c([2H])c2c(c1[2H])c1c(-c3ccccc3-c3ccccc3)cccc1n2-c1cccc(-c2nc(-c3ccc(-c4ccccc4)cc3)nc(-c3ccc4c(c3)oc3ccccc34)n2)c1 ; [2H]c1c([2H])c([2H])c2c(c1[2H])c1c(-c3ccccc3-c3ccccc3)cccc1n2-c1ccc(-c2cccc(-c3nc(-c4ccccc4)nc(-c4ccc(-c5ccccc5)cc4)n3)c2)cc1]

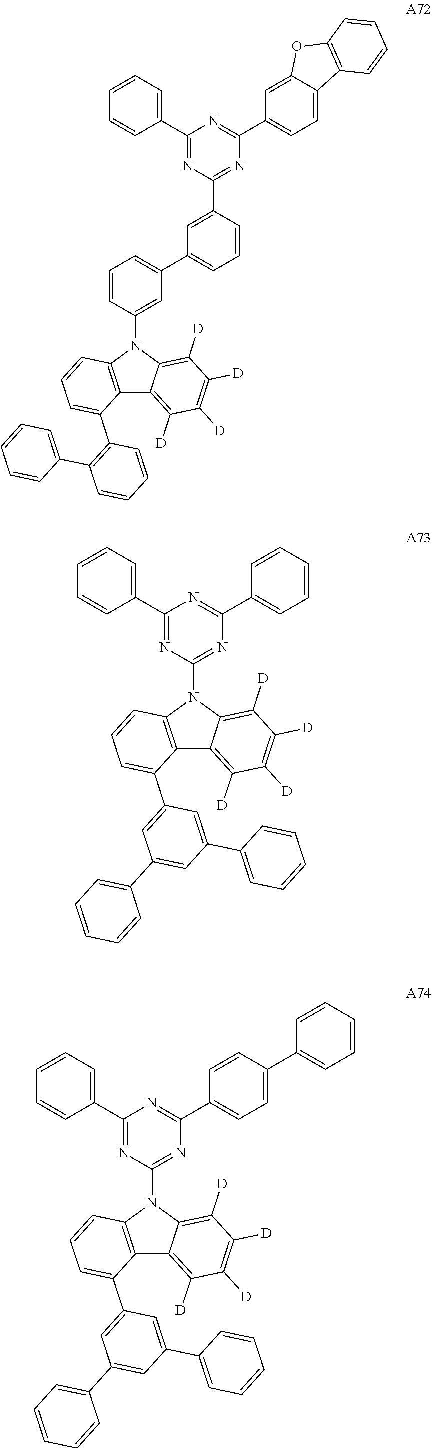 [SMILES: [2H]c1c([2H])c([2H])c2c(c1[2H])c1c(-c3ccccc3-c3ccccc3)cccc1n2-c1cccc(-c2cccc(-c3nc(-c4ccccc4)nc(-c4ccc5c(c4)oc4ccccc45)n3)c2)c1 ; [2H]c1c([2H])c([2H])c2c(c1[2H])c1c(-c3cc(-c4ccccc4)cc(-c4ccccc4)c3)cccc1n2-c1nc(-c2ccccc2)nc(-c2ccccc2)n1 ; [2H]c1c([2H])c([2H])c2c(c1[2H])c1c(-c3cc(-c4ccccc4)cc(-c4ccccc4)c3)cccc1n2-c1nc(-c2ccccc2)nc(-c2ccc(-c3ccccc3)cc2)n1]

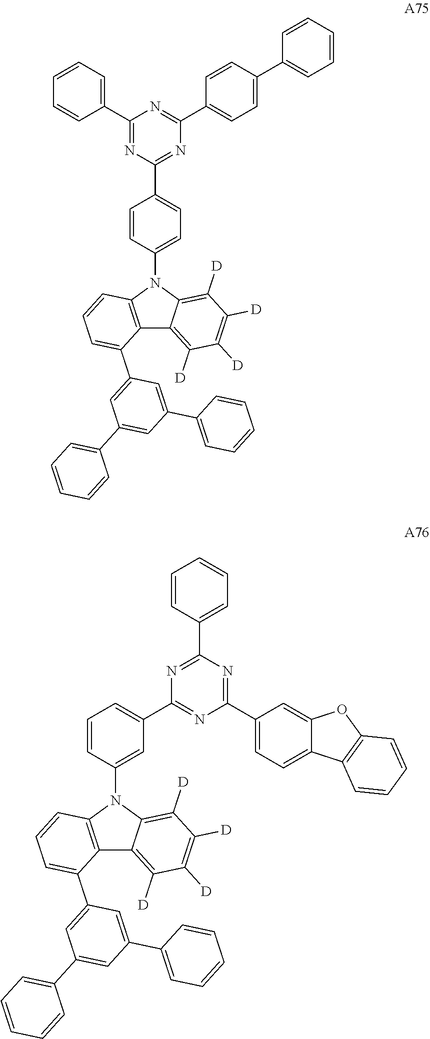 [SMILES: [2H]c1c([2H])c([2H])c2c(c1[2H])c1c(-c3cc(-c4ccccc4)cc(-c4ccccc4)c3)cccc1n2-c1ccc(-c2nc(-c3ccccc3)nc(-c3ccc(-c4ccccc4)cc3)n2)cc1 ; [2H]c1c([2H])c([2H])c2c(c1[2H])c1c(-c3cc(-c4ccccc4)cc(-c4ccccc4)c3)cccc1n2-c1cccc(-c2nc(-c3ccccc3)nc(-c3ccc4c(c3)oc3ccccc34)n2)c1]

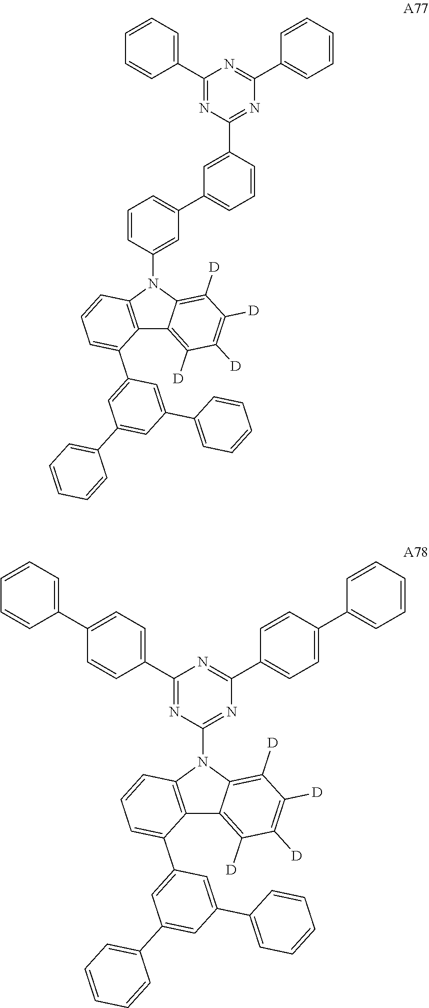 [SMILES: [2H]c1c([2H])c([2H])c2c(c1[2H])c1c(-c3cc(-c4ccccc4)cc(-c4ccccc4)c3)cccc1n2-c1cccc(-c2cccc(-c3nc(-c4ccccc4)nc(-c4ccccc4)n3)c2)c1 ; [2H]c1c([2H])c([2H])c2c(c1[2H])c1c(-c3cc(-c4ccccc4)cc(-c4ccccc4)c3)cccc1n2-c1nc(-c2ccc(-c3ccccc3)cc2)nc(-c2ccc(-c3ccccc3)cc2)n1]

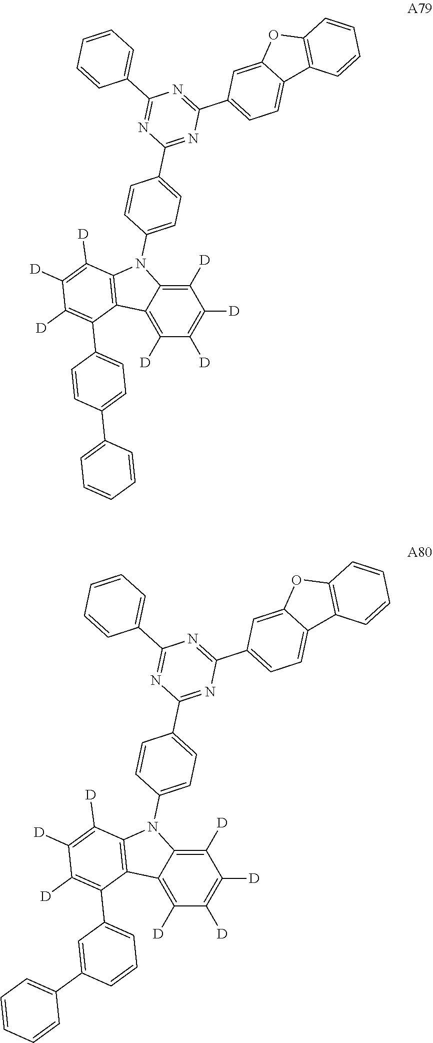 [SMILES: [2H]c1c([2H])c([2H])c2c(c1[2H])c1c(-c3ccc(-c4ccccc4)cc3)c([2H])c([2H])c([2H])c1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3cccc(-c4ccccc4)c3)c([2H])c([2H])c([2H])c1n2-c1ccc(-c2nc(-c3ccccc3)nc(-c3ccc4c(c3)oc3ccccc34)n2)cc1]

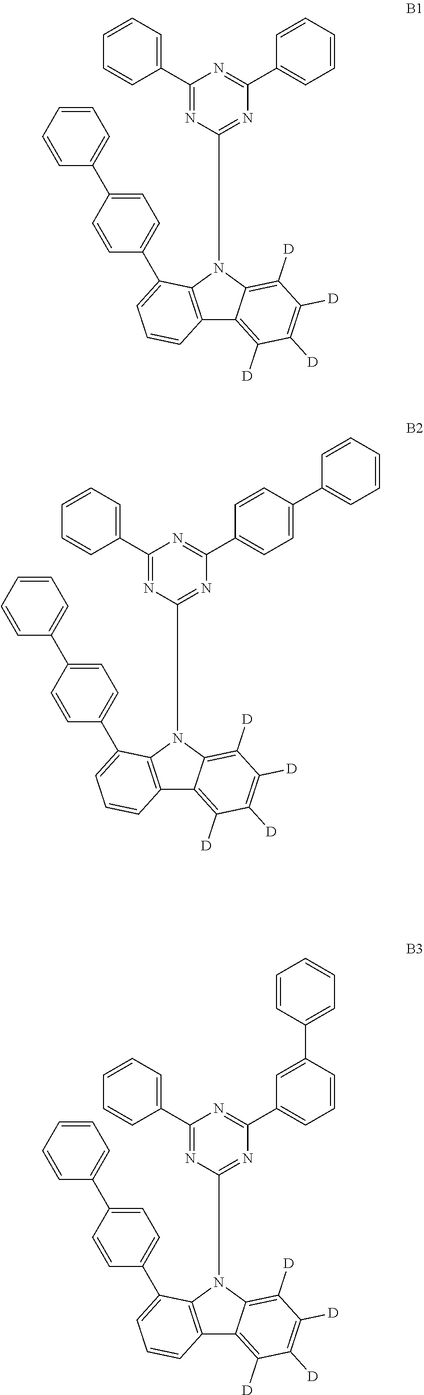 [SMILES: [2H]c1c([2H])c([2H])c2c(c1[2H])c1cccc(-c3ccc(-c4ccccc4)cc3)c1n2-c1nc(-c2ccccc2)nc(-c2ccccc2)n1 ; [2H]c1c([2H])c([2H])c2c(c1[2H])c1cccc(-c3ccc(-c4ccccc4)cc3)c1n2-c1nc(-c2ccccc2)nc(-c2ccc(-c3ccccc3)cc2)n1 ; [2H]c1c([2H])c([2H])c2c(c1[2H])c1cccc(-c3ccc(-c4ccccc4)cc3)c1n2-c1nc(-c2ccccc2)nc(-c2cccc(-c3ccccc3)c2)n1]

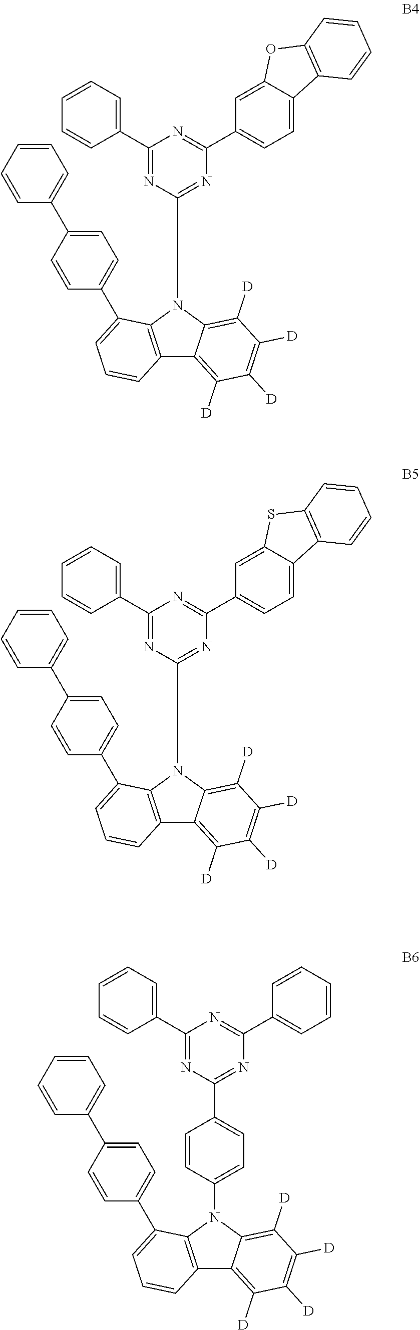 [SMILES: [2H]c1c([2H])c([2H])c2c(c1[2H])c1cccc(-c3ccc(-c4ccccc4)cc3)c1n2-c1nc(-c2ccccc2)nc(-c2ccc3c(c2)oc2ccccc23)n1 ; [2H]c1c([2H])c([2H])c2c(c1[2H])c1cccc(-c3ccc(-c4ccccc4)cc3)c1n2-c1nc(-c2ccccc2)nc(-c2ccc3c(c2)sc2ccccc23)n1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccccc3)n2)cc1]

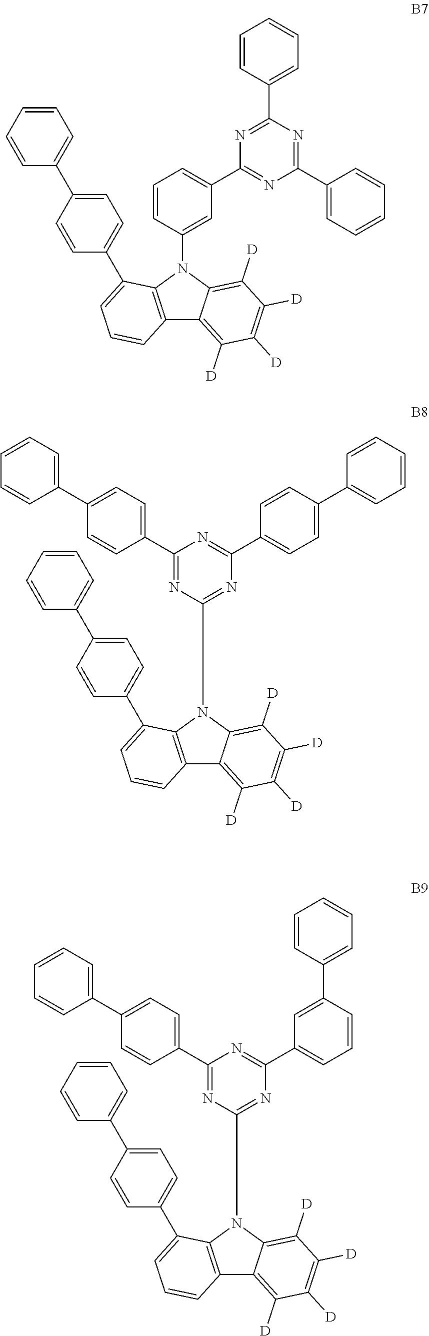 [SMILES: [2H]c1c([2H])c([2H])c2c(c1[2H])c1cccc(-c3ccc(-c4ccccc4)cc3)c1n2-c1cccc(-c2nc(-c3ccccc3)nc(-c3ccccc3)n2)c1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc(-c3ccccc3)c2)n1]

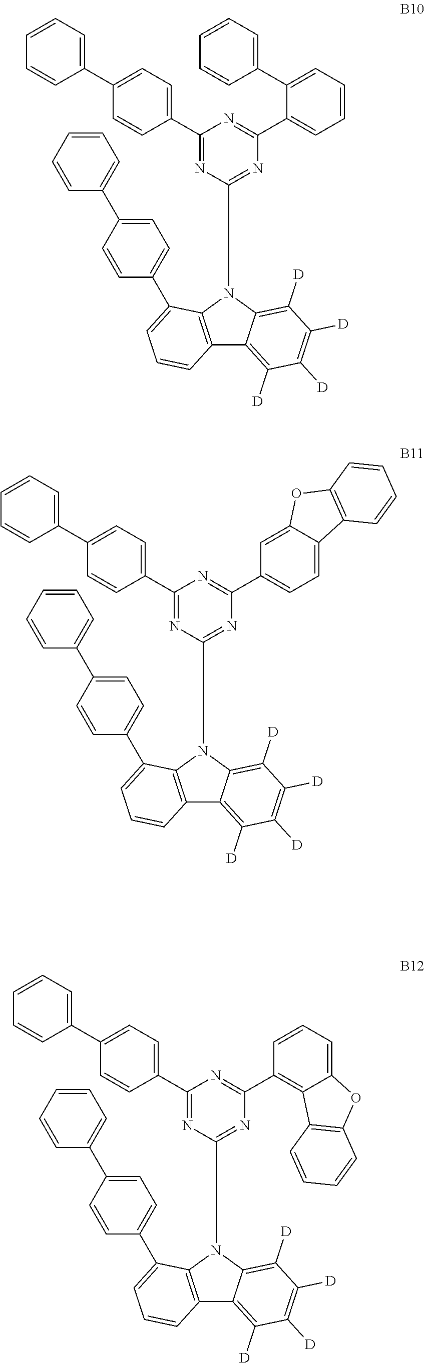 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccccc2-c2ccccc2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc3oc4ccccc4c23)n1]

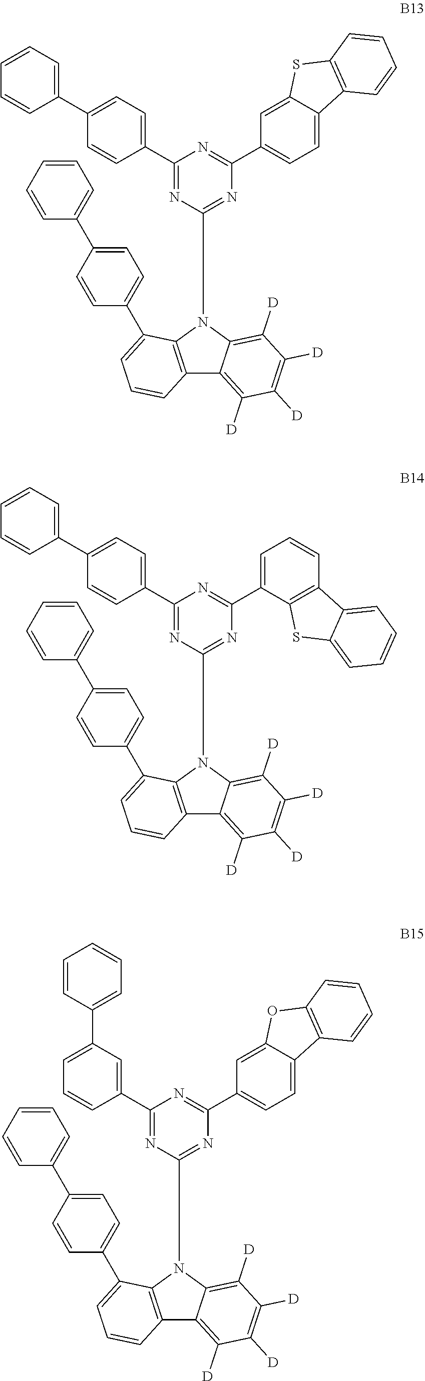 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc3c(c2)sc2ccccc23)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc3c2sc2ccccc23)n1 ; [2H]c1c([2H])c([2H])c2c(c1[2H])c1cccc(-c3ccc(-c4ccccc4)cc3)c1n2-c1nc(-c2cccc(-c3ccccc3)c2)nc(-c2ccc3c(c2)oc2ccccc23)n1]

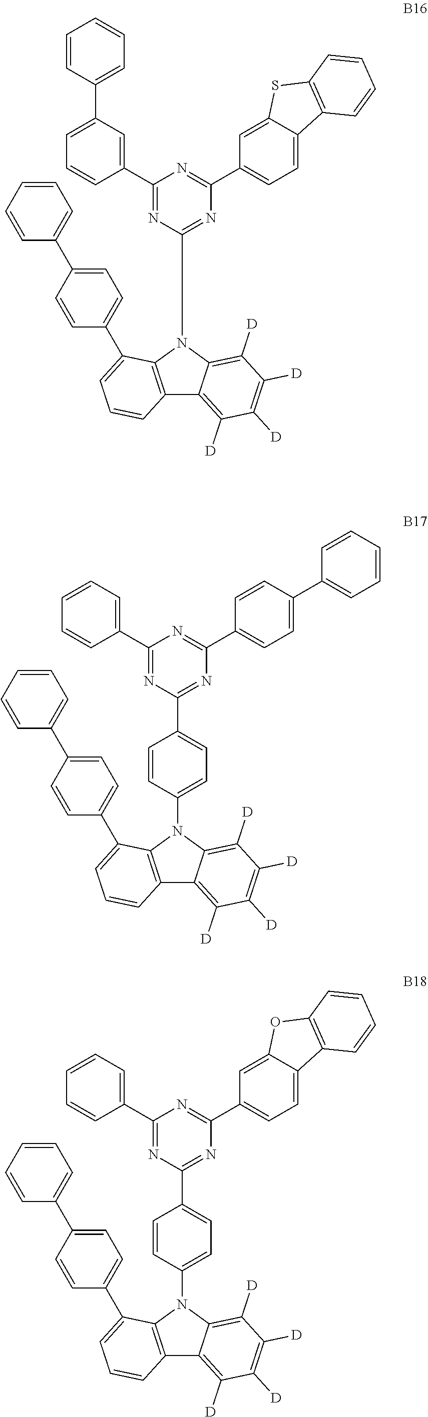 [SMILES: [2H]c1c([2H])c([2H])c2c(c1[2H])c1cccc(-c3ccc(-c4ccccc4)cc3)c1n2-c1nc(-c2cccc(-c3ccccc3)c2)nc(-c2ccc3c(c2)sc2ccccc23)n1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccc(-c4ccccc4)cc3)n2)cc1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccc4c(c3)oc3ccccc34)n2)cc1]

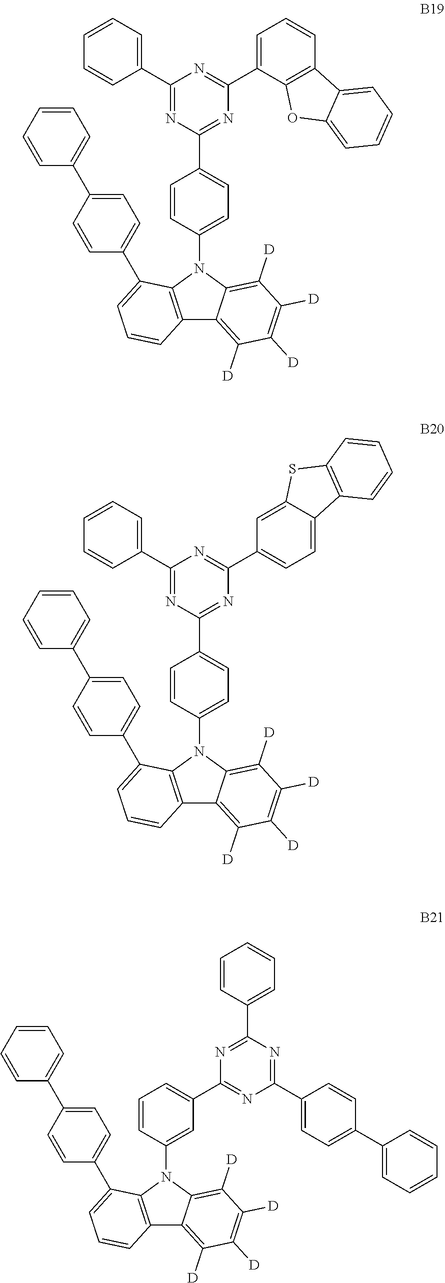 [SMILES: [2H]c1c([2H])c([2H])c2c(c1[2H])c1cccc(-c3ccc(-c4ccccc4)cc3)c1n2-c1ccc(-c2nc(-c3ccccc3)nc(-c3cccc4c3oc3ccccc34)n2)cc1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccc(-c4ccccc4)cc3)n2)c1]

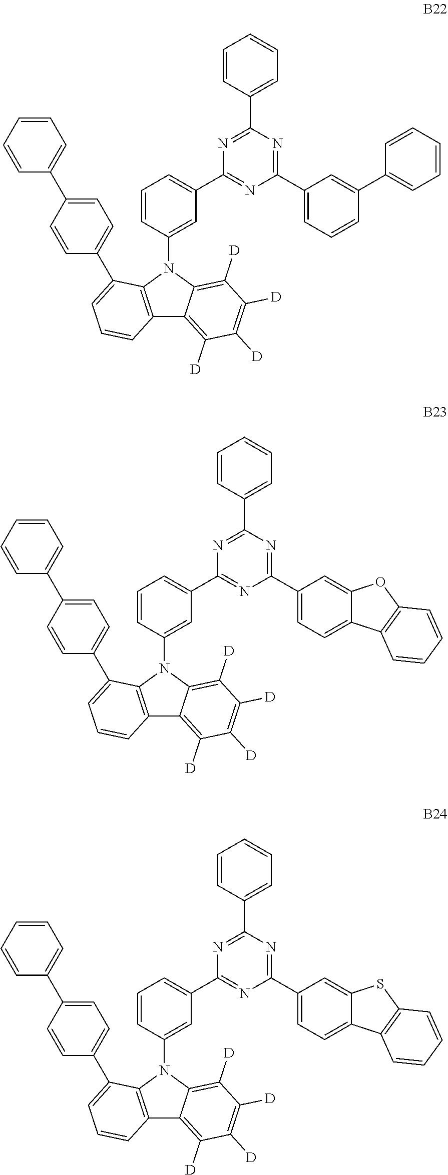 [SMILES: [2H]c1c([2H])c([2H])c2c(c1[2H])c1cccc(-c3ccc(-c4ccccc4)cc3)c1n2-c1cccc(-c2nc(-c3ccccc3)nc(-c3cccc(-c4ccccc4)c3)n2)c1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccc4c(c3)oc3ccccc34)n2)c1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccc4c(c3)sc3ccccc34)n2)c1]

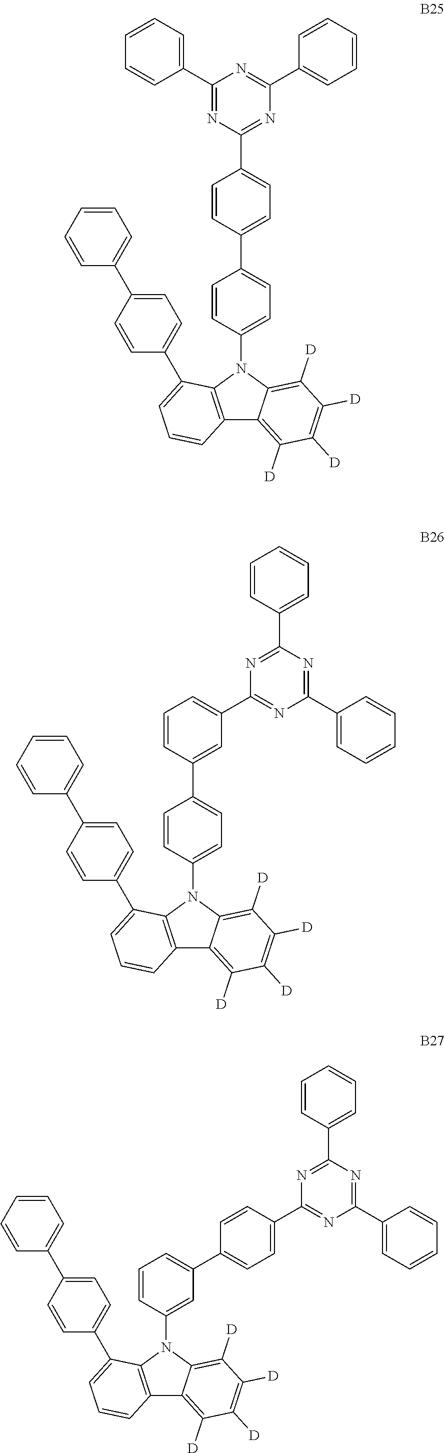 [SMILES: [2H]c1c([2H])c([2H])c2c(c1[2H])c1cccc(-c3ccc(-c4ccccc4)cc3)c1n2-c1ccc(-c2ccc(-c3nc(-c4ccccc4)nc(-c4ccccc4)n3)cc2)cc1 ; [2H]c1c([2H])c([2H])c2c(c1[2H])c1cccc(-c3ccc(-c4ccccc4)cc3)c1n2-c1ccc(-c2cccc(-c3nc(-c4ccccc4)nc(-c4ccccc4)n3)c2)cc1 ; [2H]c1c([2H])c([2H])c2c(c1[2H])c1cccc(-c3ccc(-c4ccccc4)cc3)c1n2-c1cccc(-c2ccc(-c3nc(-c4ccccc4)nc(-c4ccccc4)n3)cc2)c1]

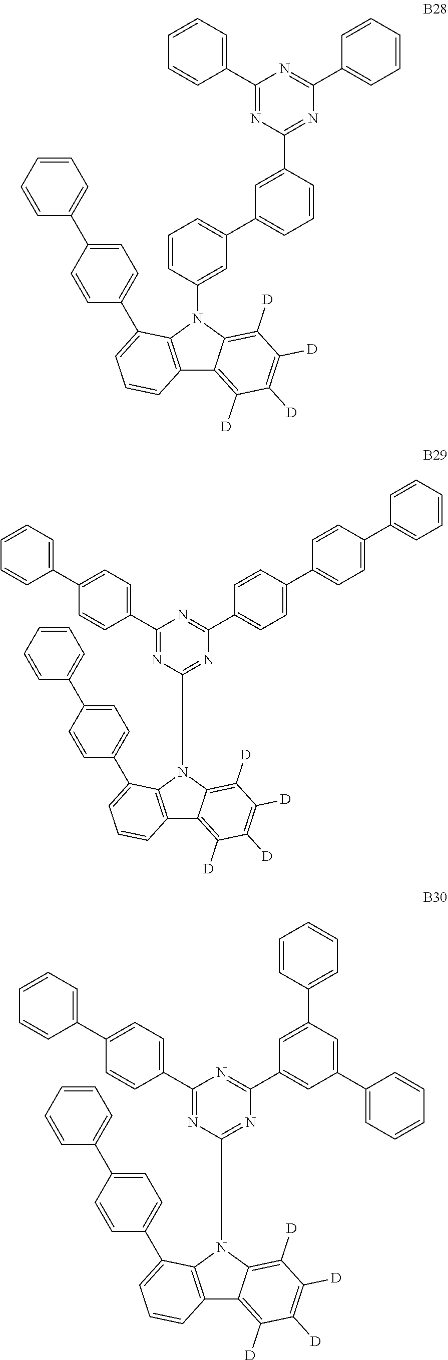 [SMILES: [2H]c1c([2H])c([2H])c2c(c1[2H])c1cccc(-c3ccc(-c4ccccc4)cc3)c1n2-c1cccc(-c2cccc(-c3nc(-c4ccccc4)nc(-c4ccccc4)n3)c2)c1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccc(-c4ccccc4)cc3)cc2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cc(-c3ccccc3)cc(-c3ccccc3)c2)n1]

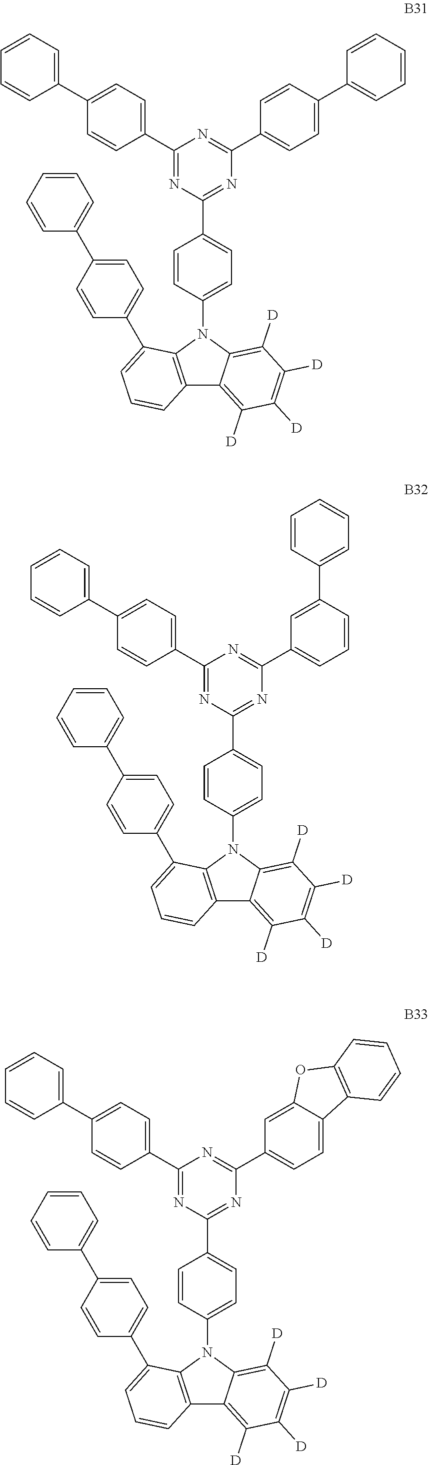 [SMILES: [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc(-c4ccccc4)cc3)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3cccc(-c4ccccc4)c3)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4c(c3)oc3ccccc34)n2)cc1]

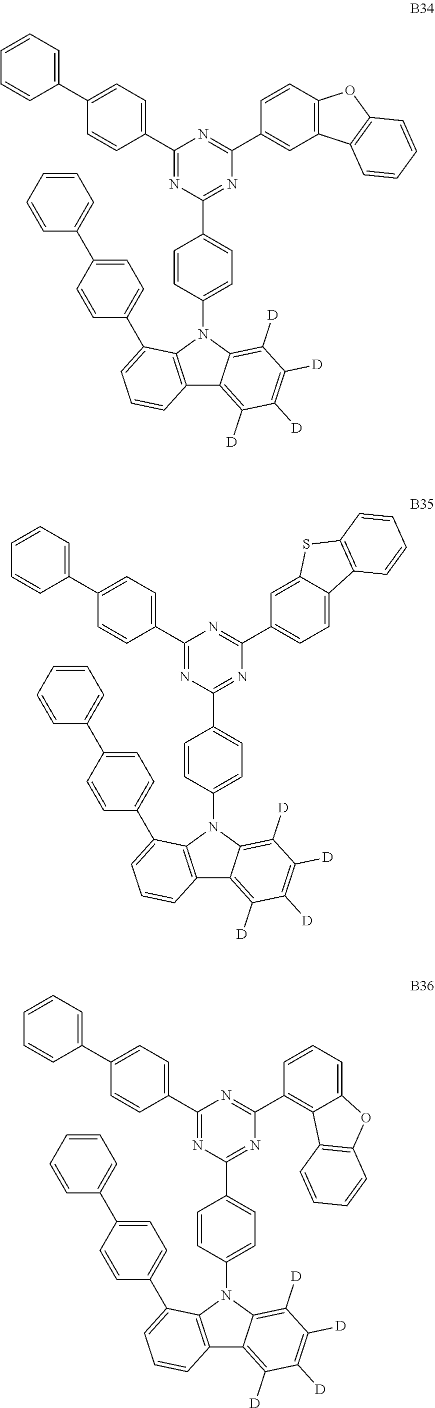 [SMILES: [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4oc5ccccc5c4c3)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4c(c3)sc3ccccc34)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3cccc4oc5ccccc5c34)n2)cc1]

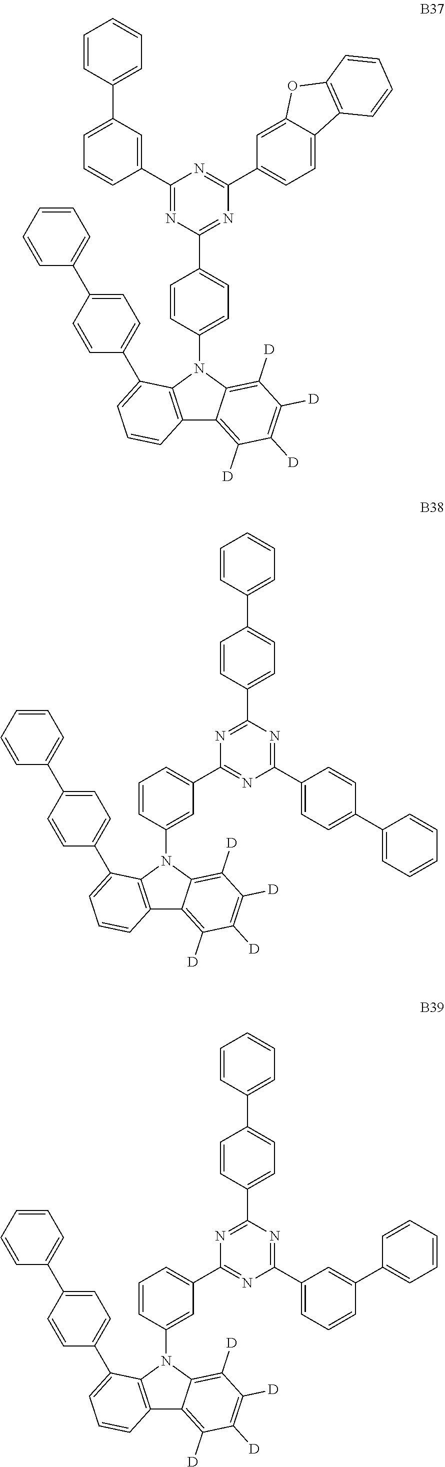 [SMILES: [2H]c1c([2H])c([2H])c2c(c1[2H])c1cccc(-c3ccc(-c4ccccc4)cc3)c1n2-c1ccc(-c2nc(-c3cccc(-c4ccccc4)c3)nc(-c3ccc4c(c3)oc3ccccc34)n2)cc1 ; [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3ccc(-c4ccccc4)cc3)n2)c1 ; [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3cccc(-c4ccccc4)c3)n2)c1]

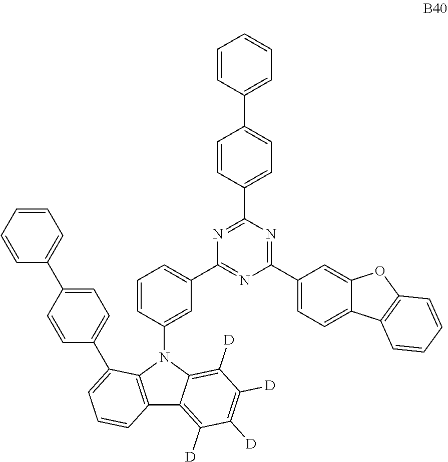 [SMILES: [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3ccc4c(c3)oc3ccccc34)n2)c1]

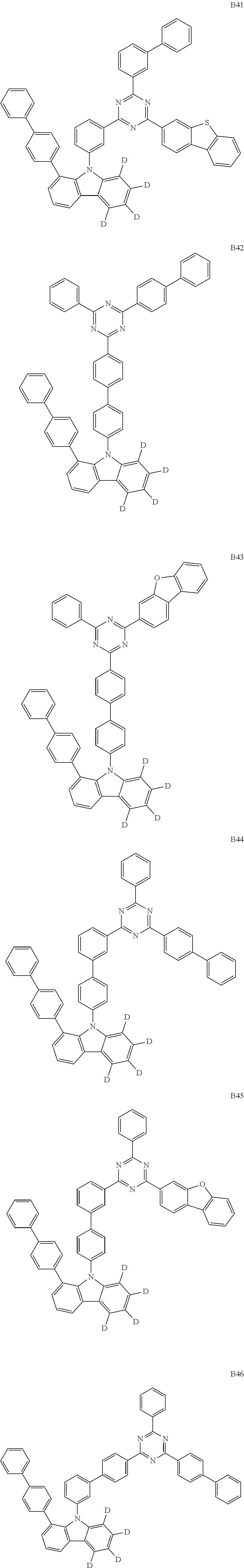

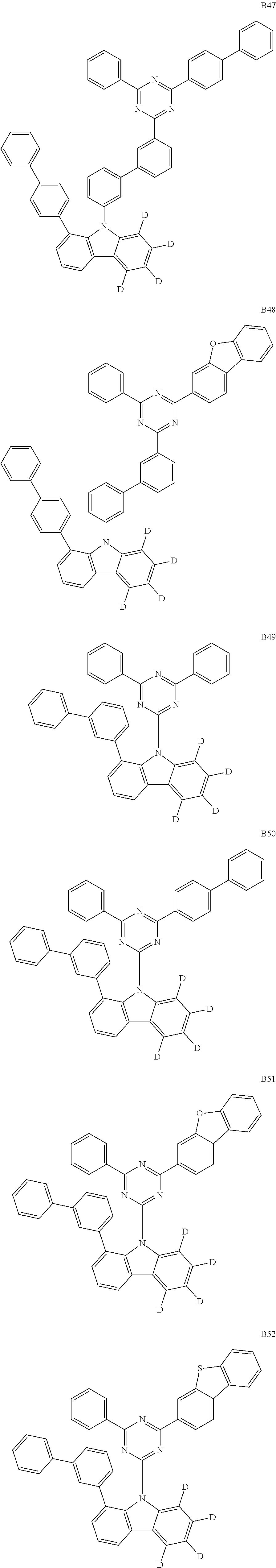

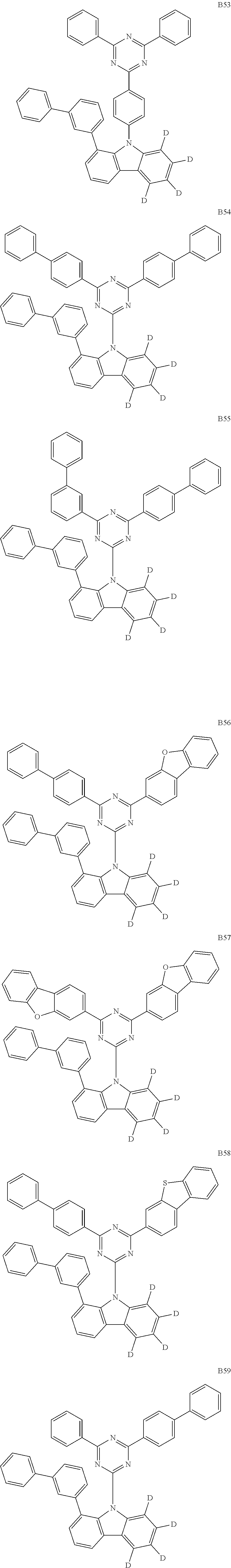

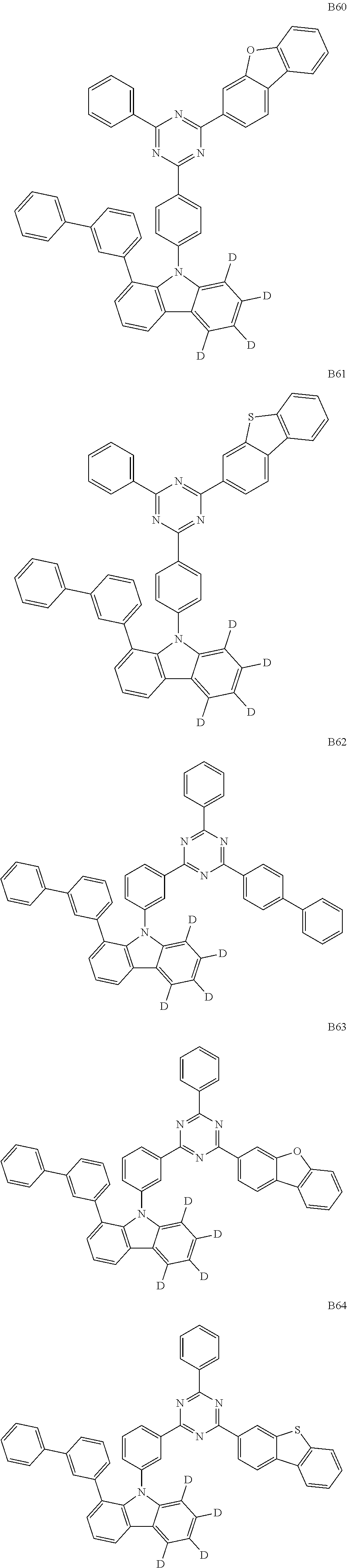

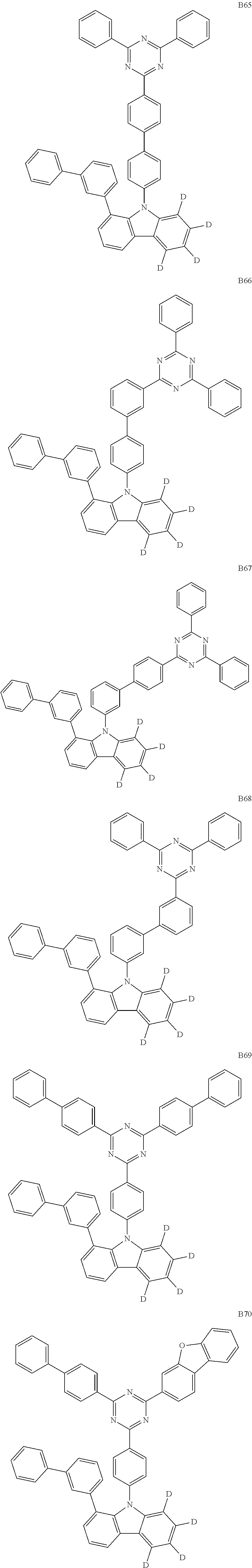

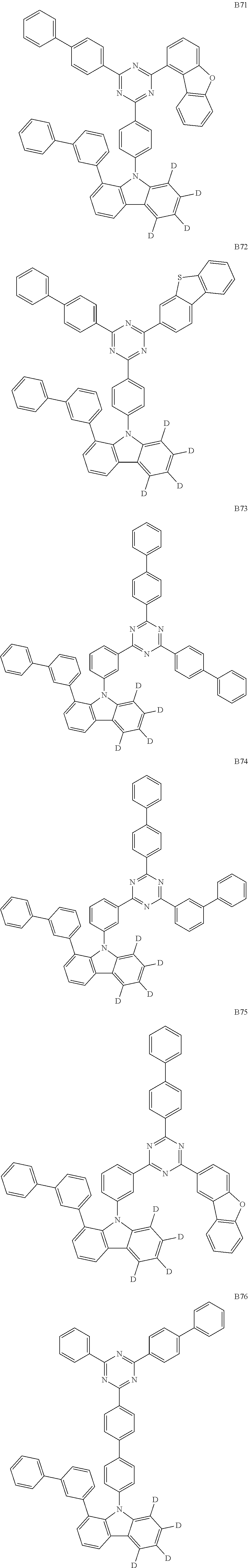

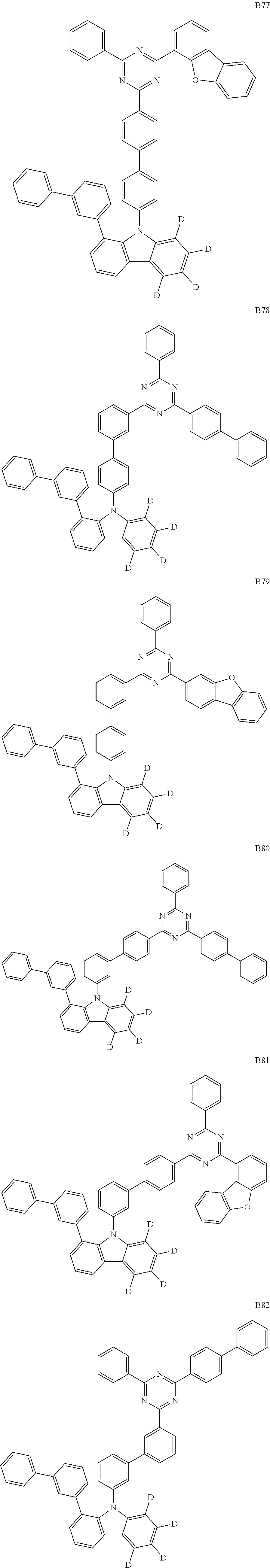

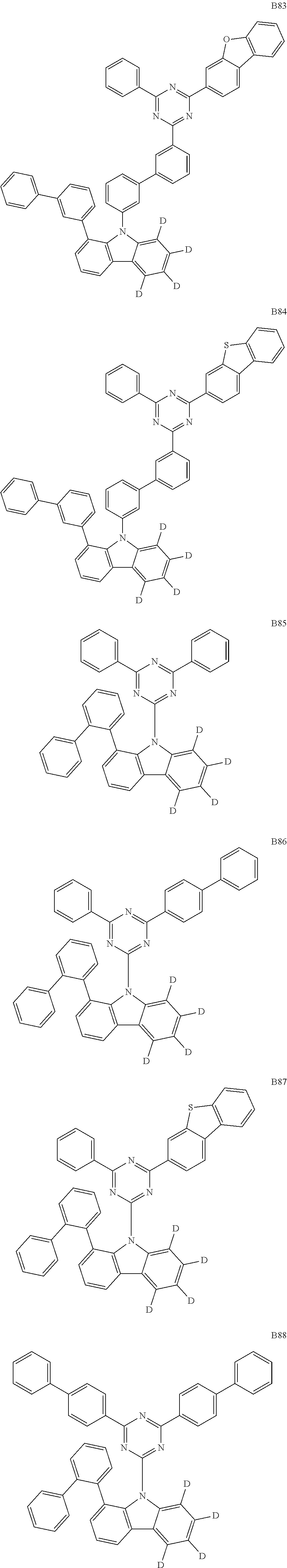

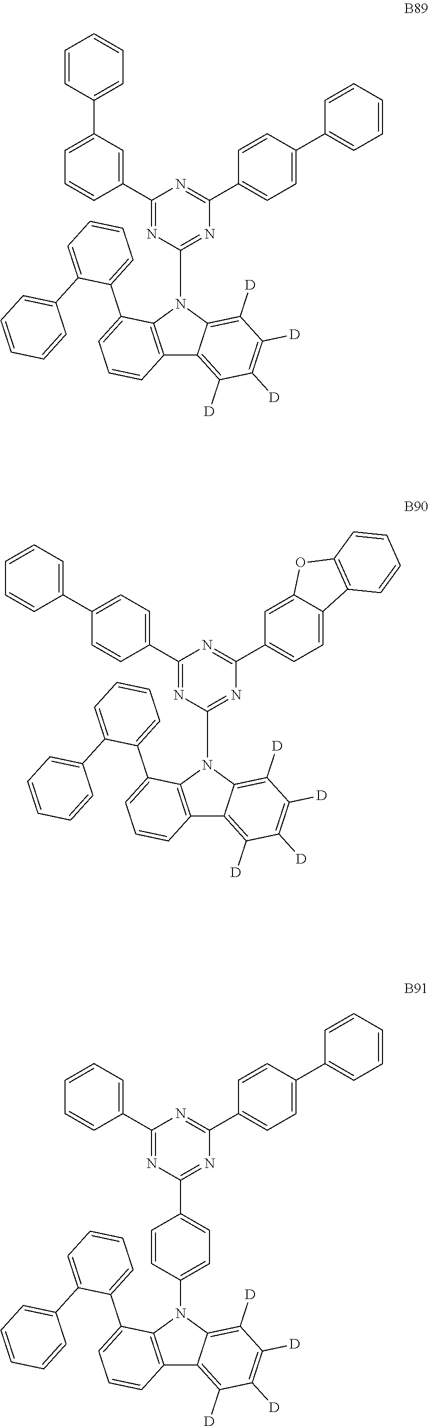 [SMILES: [2H]c1c([2H])c([2H])c2c(c1[2H])c1cccc(-c3ccccc3-c3ccccc3)c1n2-c1nc(-c2ccc(-c3ccccc3)cc2)nc(-c2cccc(-c3ccccc3)c2)n1 ; [2H]c1c([2H])c([2H])c2c(c1[2H])c1cccc(-c3ccccc3-c3ccccc3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3ccccc3-c3ccccc3)c1n2-c1ccc(-c2nc(-c3ccccc3)nc(-c3ccc(-c4ccccc4)cc3)n2)cc1]

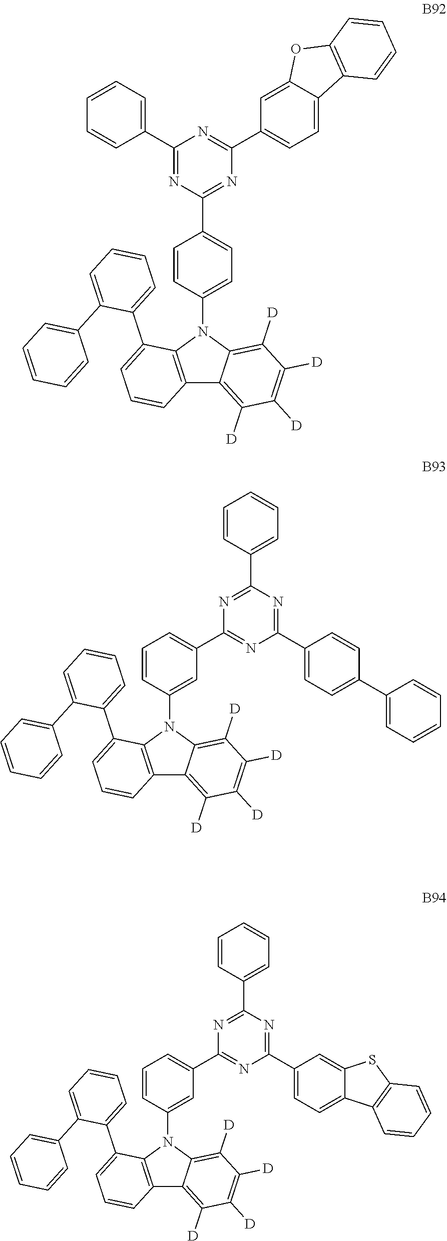 [SMILES: [2H]c1c([2H])c([2H])c2c(c1[2H])c1cccc(-c3ccccc3-c3ccccc3)c1n2-c1ccc(-c2nc(-c3ccccc3)nc(-c3ccc4c(c3)oc3ccccc34)n2)cc1 ; [2H]c1c([2H])c([2H])c2c(c1[2H])c1cccc(-c3ccccc3-c3ccccc3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3ccccc3-c3ccccc3)c1n2-c1cccc(-c2nc(-c3ccccc3)nc(-c3ccc4c(c3)sc3ccccc34)n2)c1]

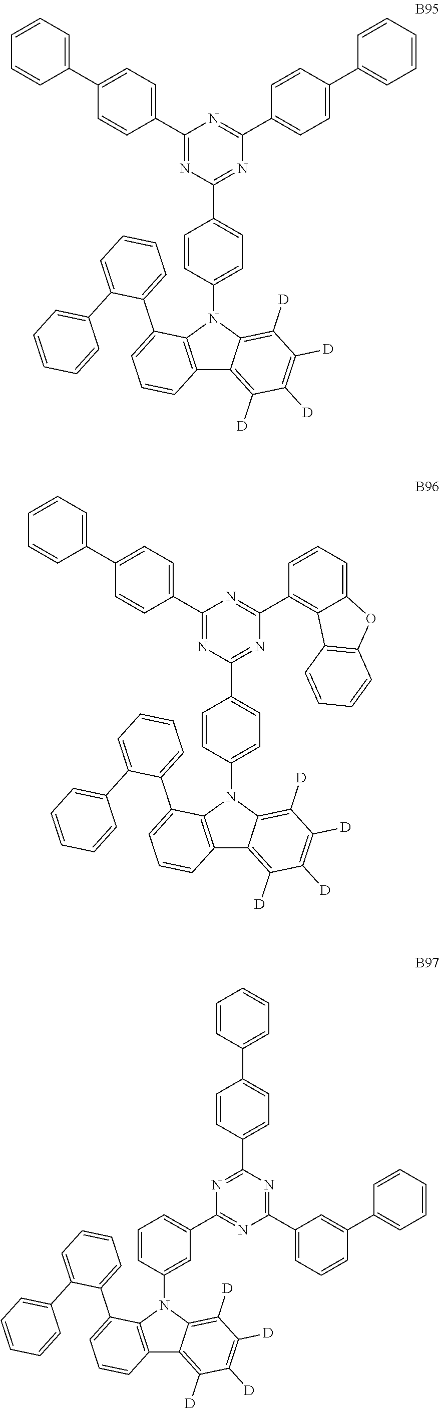 [SMILES: [2H]c1c([2H])c([2H])c2c(c1[2H])c1cccc(-c3ccccc3-c3ccccc3)c1n2-c1ccc(-c2nc(-c3ccc(-c4ccccc4)cc3)nc(-c3ccc(-c4ccccc4)cc3)n2)cc1 ; [2H]c1c([2H])c([2H])c2c(c1[2H])c1cccc(-c3ccccc3-c3ccccc3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3ccccc3-c3ccccc3)c1n2-c1cccc(-c2nc(-c3ccc(-c4ccccc4)cc3)nc(-c3cccc(-c4ccccc4)c3)n2)c1]

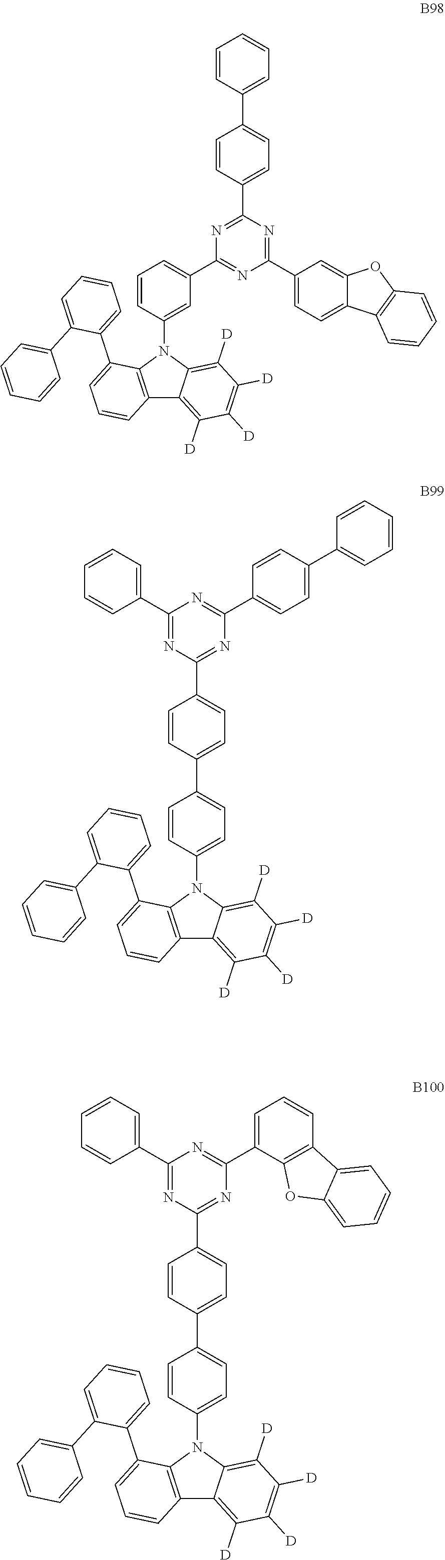 [SMILES: [2H]c1c([2H])c([2H])c2c(c1[2H])c1cccc(-c3ccccc3-c3ccccc3)c1n2-c1cccc(-c2nc(-c3ccc(-c4ccccc4)cc3)nc(-c3ccc4c(c3)oc3ccccc34)n2)c1 ; [2H]c1c([2H])c([2H])c2c(c1[2H])c1cccc(-c3ccccc3-c3ccccc3)c1n2-c1ccc(-c2ccc(-c3nc(-c4ccccc4)nc(-c4ccc(-c5ccccc5)cc4)n3)cc2)cc1 ; [2H]c1c([2H])c([2H])c2c(c1[2H])c1cccc(-c3ccccc3-c3ccccc3)c1n2-c1ccc(-c2ccc(-c3nc(-c4ccccc4)nc(-c4cccc5c4oc4ccccc45)n3)cc2)cc1]

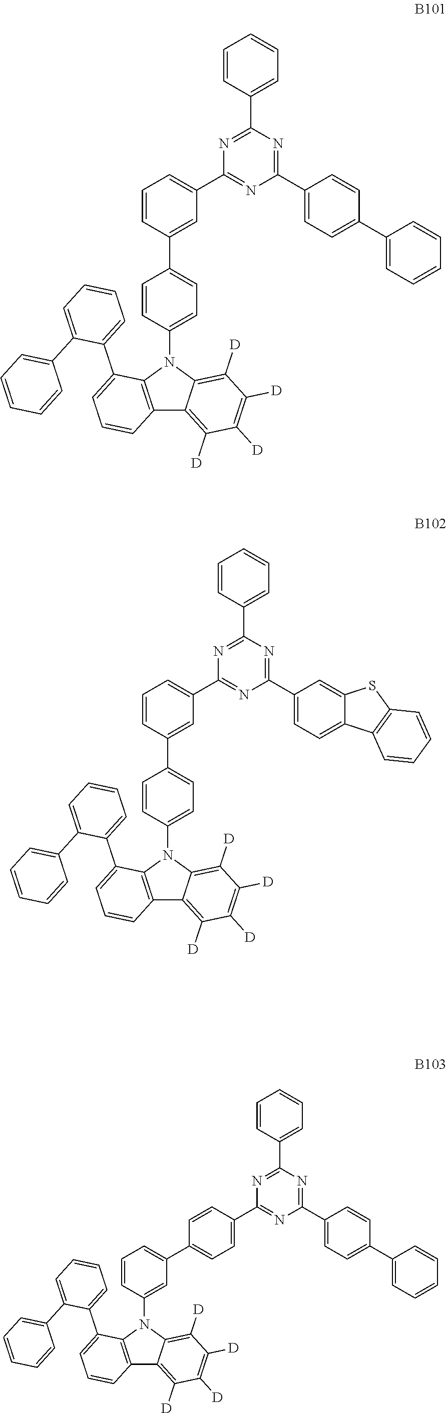 [SMILES: [2H]c1c([2H])c([2H])c2c(c1[2H])c1cccc(-c3ccccc3-c3ccccc3)c1n2-c1ccc(-c2cccc(-c3nc(-c4ccccc4)nc(-c4ccc(-c5ccccc5)cc4)n3)c2)cc1 ; [2H]c1c([2H])c([2H])c2c(c1[2H])c1cccc(-c3ccccc3-c3ccccc3)c1n2-c1ccc(-c2cccc(-c3nc(-c4ccccc4)nc(-c4ccc5c(c4)sc4ccccc45)n3)c2)cc1 ; [2H]c1c([2H])c([2H])c2c(c1[2H])c1cccc(-c3ccccc3-c3ccccc3)c1n2-c1cccc(-c2ccc(-c3nc(-c4ccccc4)nc(-c4ccc(-c5ccccc5)cc4)n3)cc2)c1]

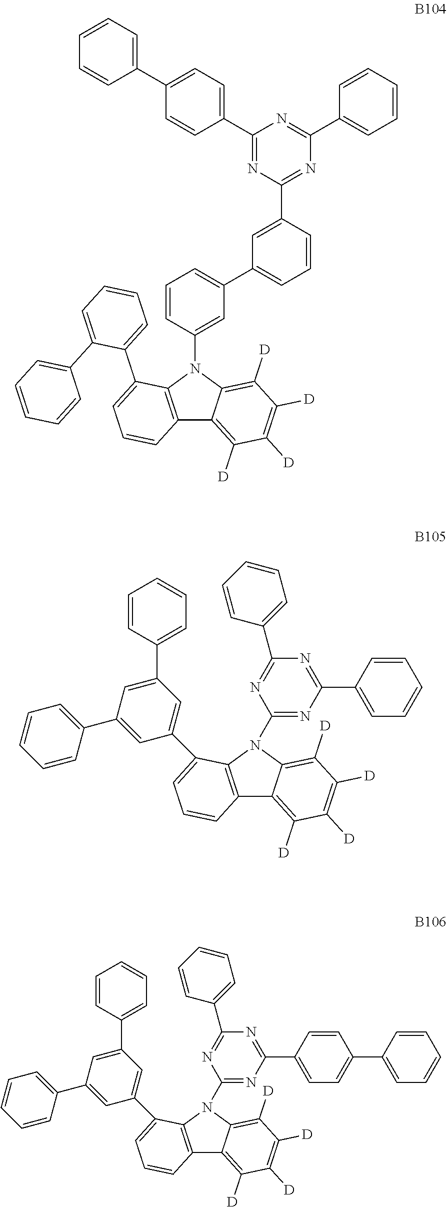 [SMILES: [2H]c1c([2H])c([2H])c2c(c1[2H])c1cccc(-c3ccccc3-c3ccccc3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccccc2)n1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc(-c3ccccc3)cc2)n1]

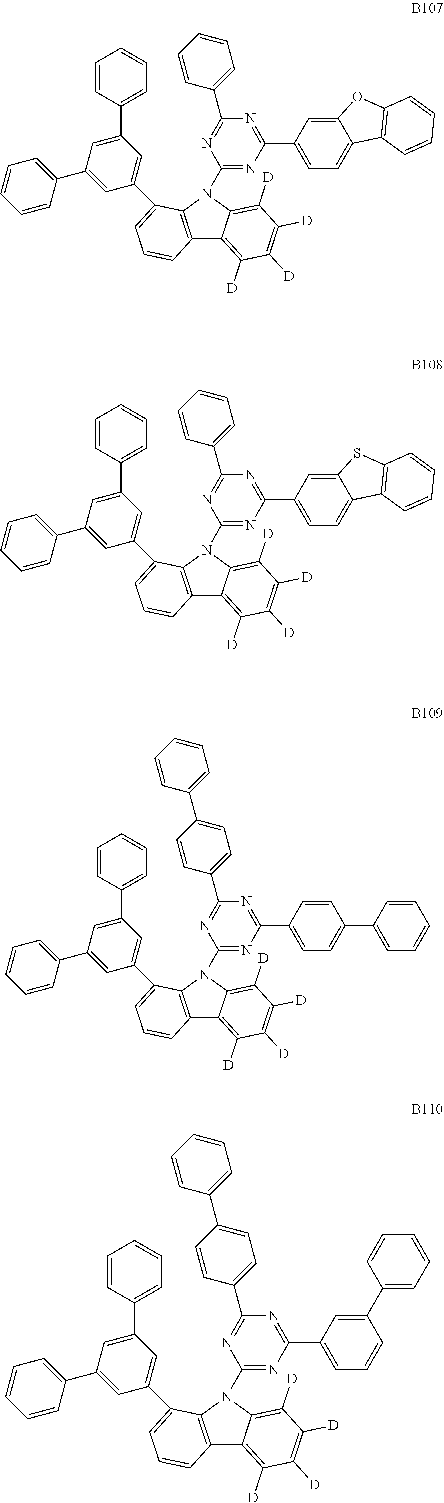 [SMILES: [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc3c(c2)oc2ccccc23)n1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc3c(c2)sc2ccccc23)n1 ; [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2cccc(-c3ccccc3)c2)n1]

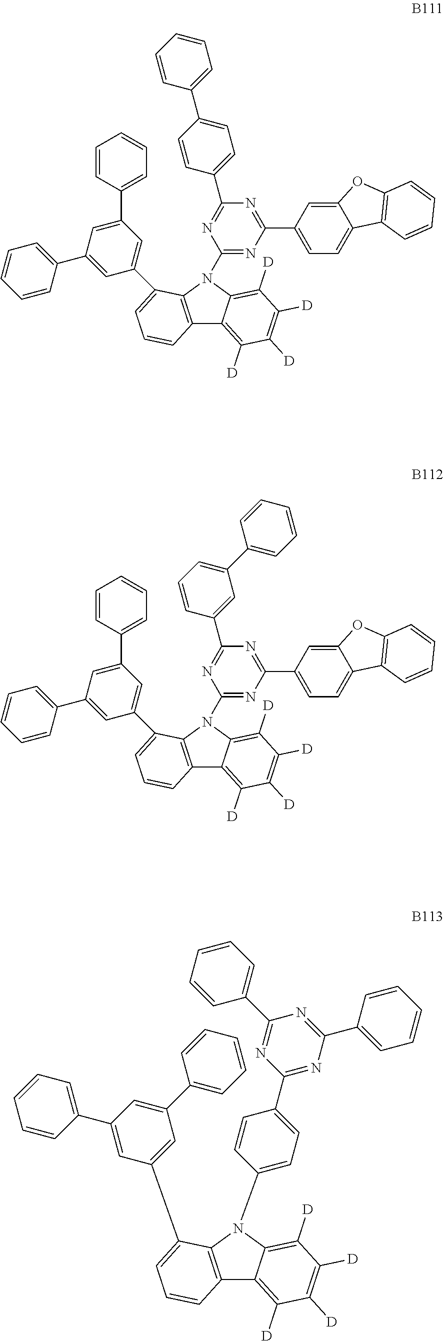 [SMILES: [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3cc(-c4ccccc4)cc(-c4ccccc4)c3)c1n2-c1nc(-c2cccc(-c3ccccc3)c2)nc(-c2ccc3c(c2)oc2ccccc23)n1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3ccccc3)n2)cc1]

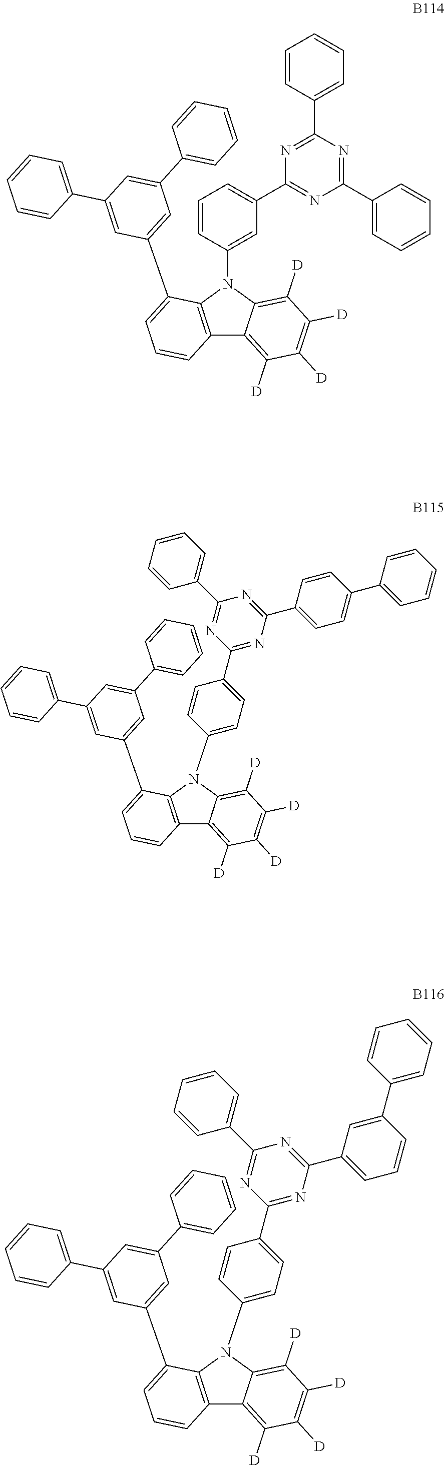 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccccc3)nc(-c3ccccc3)n2)c1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3ccc(-c4ccccc4)cc3)n2)cc1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3cccc(-c4ccccc4)c3)n2)cc1]

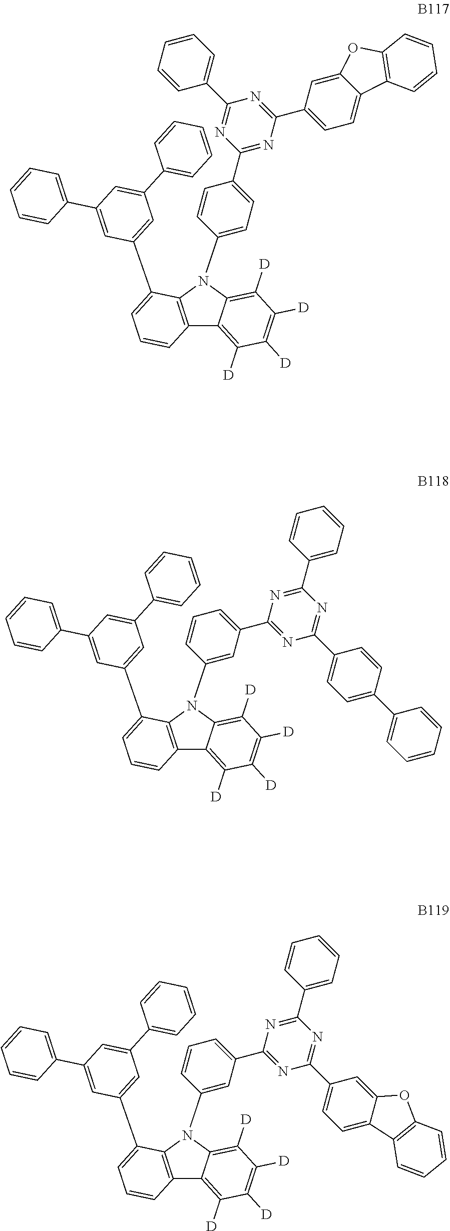 [SMILES: [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3ccc4c(c3)oc3ccccc34)n2)cc1 ; [2H]c1c([2H])c([2H])c2c(c1[2H])c1cccc(-c3cc(-c4ccccc4)cc(-c4ccccc4)c3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3cc(-c4ccccc4)cc(-c4ccccc4)c3)c1n2-c1cccc(-c2nc(-c3ccccc3)nc(-c3ccc4c(c3)oc3ccccc34)n2)c1]

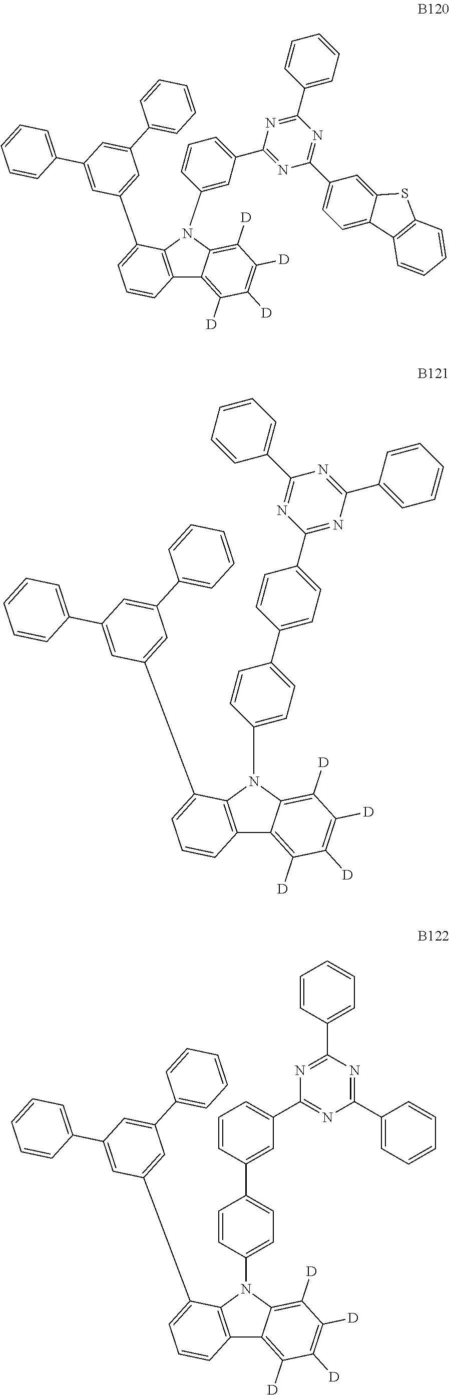 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccccc3)nc(-c3ccc4c(c3)sc3ccccc34)n2)c1 ; [2H]c1c([2H])c([2H])c2c(c1[2H])c1cccc(-c3cc(-c4ccccc4)cc(-c4ccccc4)c3)c1n2-c1ccc(-c2ccc(-c3nc(-c4ccccc4)nc(-c4ccccc4)n3)cc2)cc1 ; [2H]c1c([2H])c([2H])c2c(c1[2H])c1cccc(-c3cc(-c4ccccc4)cc(-c4ccccc4)c3)c1n2-c1ccc(-c2cccc(-c3nc(-c4ccccc4)nc(-c4ccccc4)n3)c2)cc1]

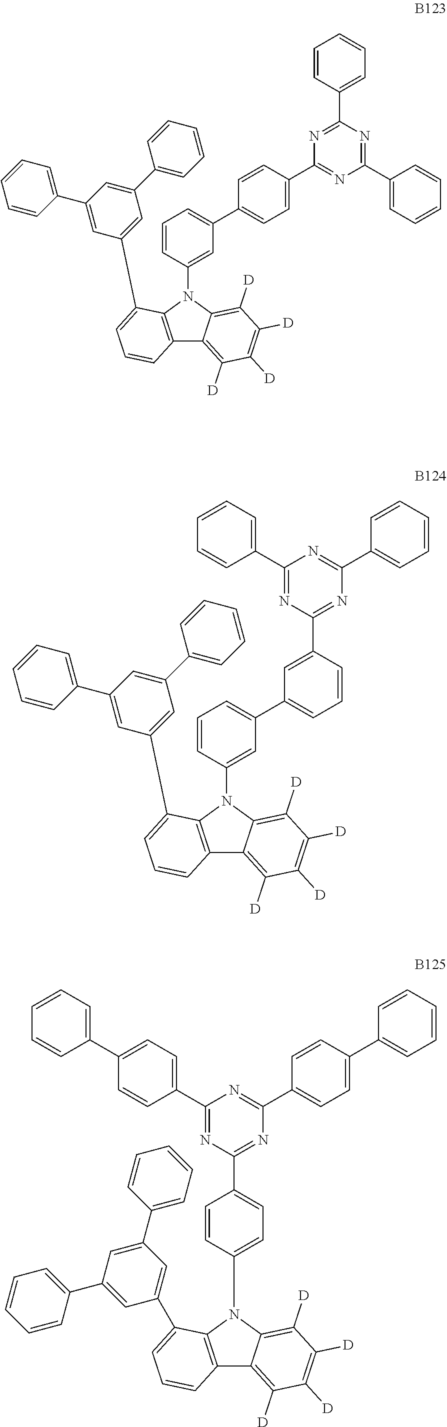 [SMILES: [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccccc4)n3)cc2)c1 ; [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccccc4)n3)c2)c1 ; [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3ccc(-c4ccccc4)cc3)n2)cc1]

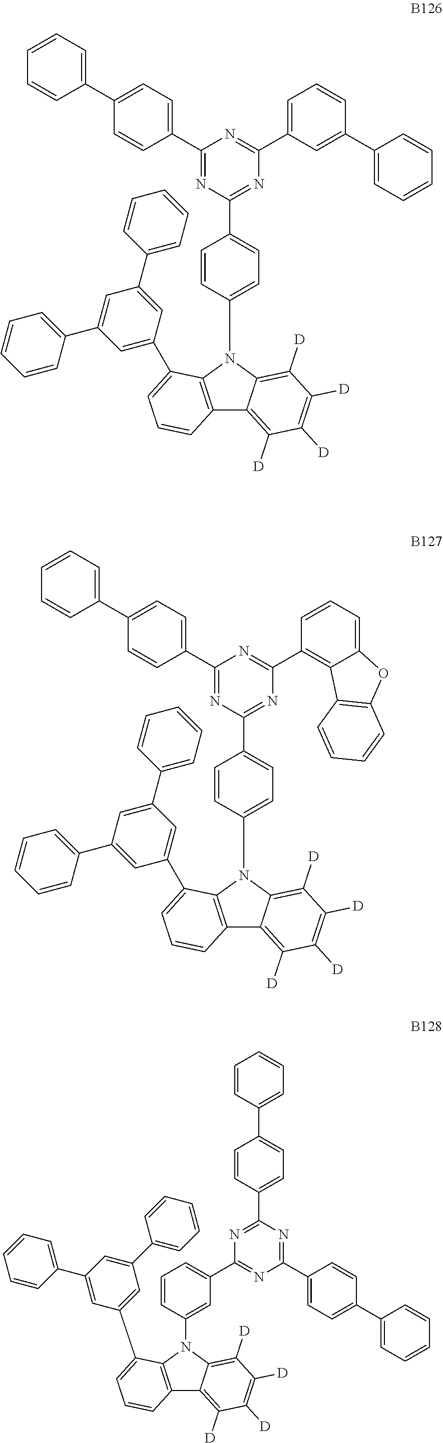 [SMILES: [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3cccc(-c4ccccc4)c3)n2)cc1 ; [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3ccc(-c4ccccc4)cc3)n2)c1]

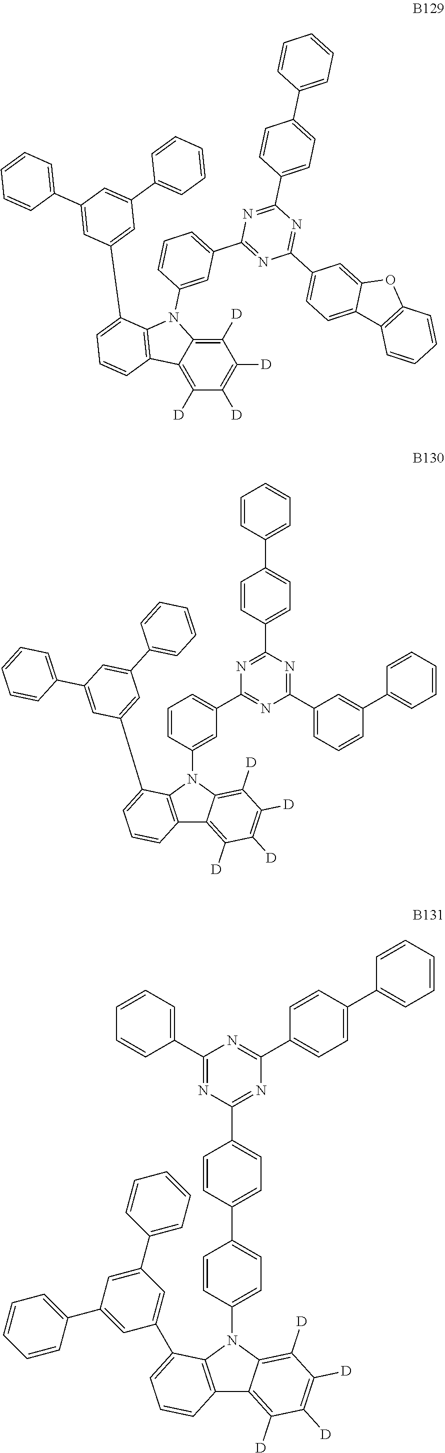 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3ccc4c(c3)oc3ccccc34)n2)c1 ; [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3cccc(-c4ccccc4)c3)n2)c1 ; [2H]c1c([2H])c([2H])c2c(c1[2H])c1cccc(-c3cc(-c4ccccc4)cc(-c4ccccc4)c3)c1n2-c1ccc(-c2ccc(-c3nc(-c4ccccc4)nc(-c4ccc(-c5ccccc5)cc4)n3)cc2)cc1]

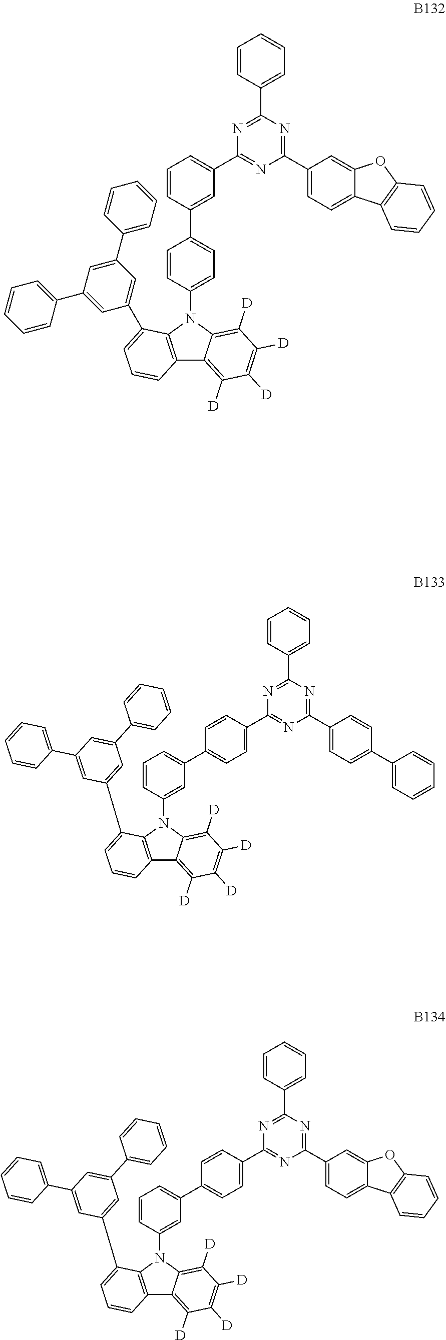 [SMILES: [2H]c1c([2H])c([2H])c2c(c1[2H])c1cccc(-c3cc(-c4ccccc4)cc(-c4ccccc4)c3)c1n2-c1ccc(-c2cccc(-c3nc(-c4ccccc4)nc(-c4ccc5c(c4)oc4ccccc45)n3)c2)cc1 ; [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccc(-c5ccccc5)cc4)n3)cc2)c1 ; [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccc5c(c4)oc4ccccc45)n3)cc2)c1]

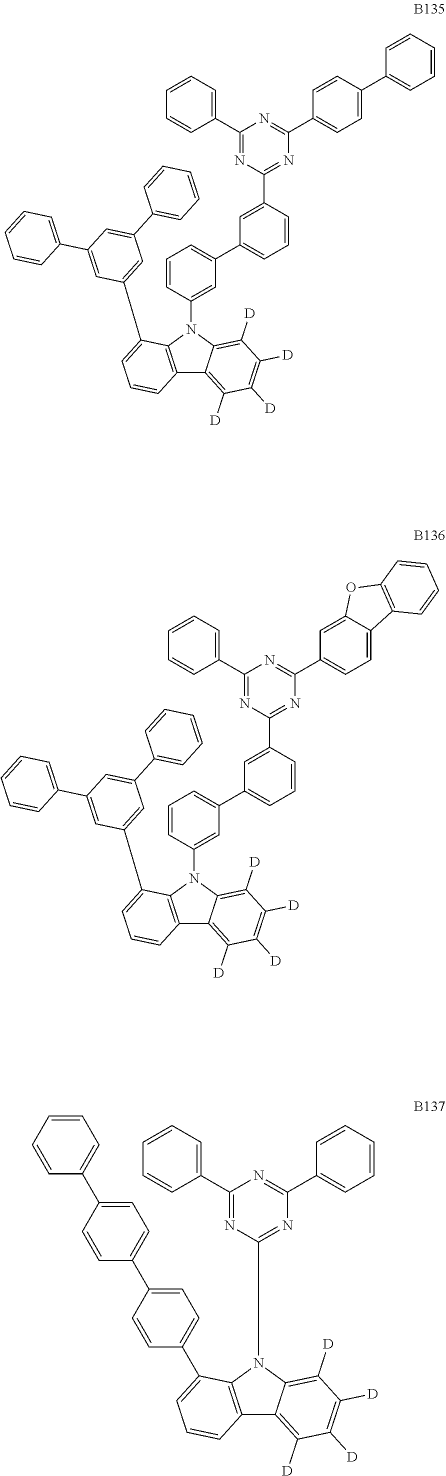 [SMILES: [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccc5c(c4)oc4ccccc45)n3)c2)c1 ; [2H]c1c([2H])c([2H])c2c(c1[2H])c1cccc(-c3ccc(-c4ccc(-c5ccccc5)cc4)cc3)c1n2-c1nc(-c2ccccc2)nc(-c2ccccc2)n1]

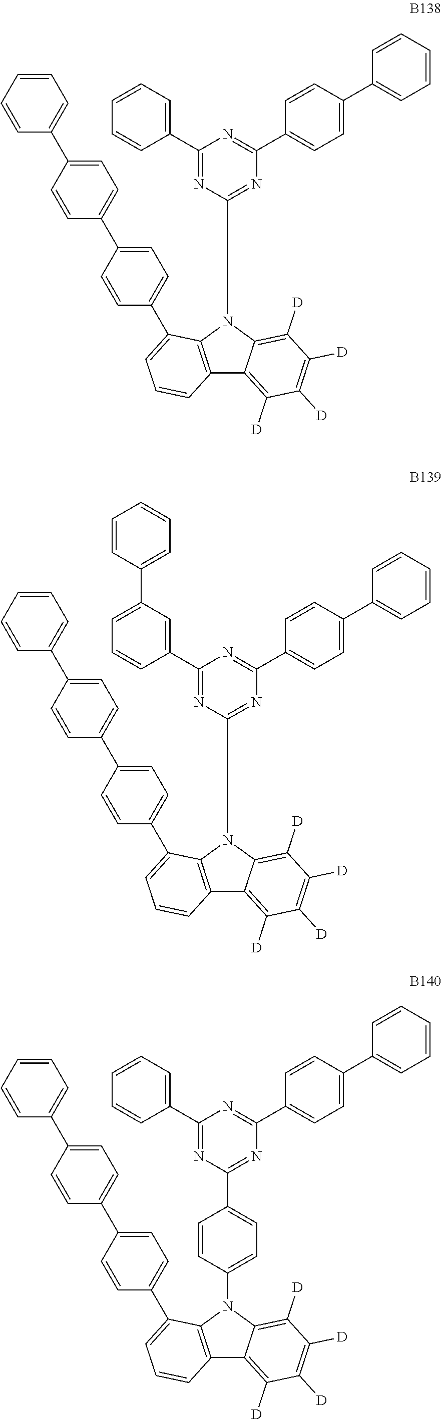 [SMILES: [2H]c1c([2H])c([2H])c2c(c1[2H])c1cccc(-c3ccc(-c4ccc(-c5ccccc5)cc4)cc3)c1n2-c1nc(-c2ccccc2)nc(-c2ccc(-c3ccccc3)cc2)n1 ; [2H]c1c([2H])c([2H])c2c(c1[2H])c1cccc(-c3ccc(-c4ccc(-c5ccccc5)cc4)cc3)c1n2-c1nc(-c2ccc(-c3ccccc3)cc2)nc(-c2cccc(-c3ccccc3)c2)n1 ; [2H]c1c([2H])c([2H])c2c(c1[2H])c1cccc(-c3ccc(-c4ccc(-c5ccccc5)cc4)cc3)c1n2-c1ccc(-c2nc(-c3ccccc3)nc(-c3ccc(-c4ccccc4)cc3)n2)cc1]

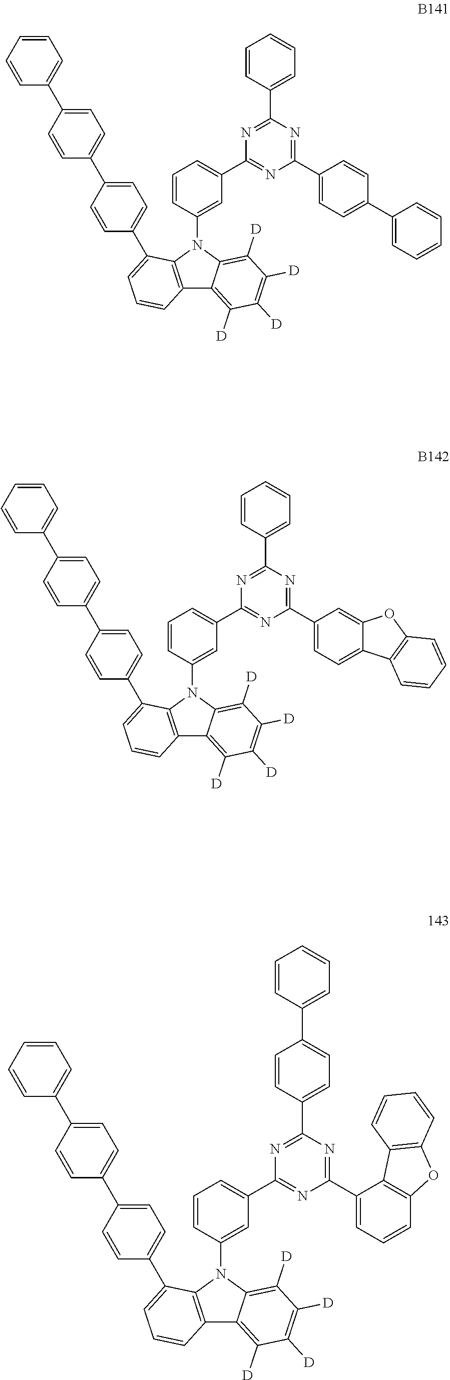 [SMILES: [2H]c1c([2H])c([2H])c2c(c1[2H])c1cccc(-c3ccc(-c4ccc(-c5ccccc5)cc4)cc3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3ccc(-c4ccc(-c5ccccc5)cc4)cc3)c1n2-c1cccc(-c2nc(-c3ccccc3)nc(-c3ccc4c(c3)oc3ccccc34)n2)c1 ; [2H]c1c([2H])c([2H])c2c(c1[2H])c1cccc(-c3ccc(-c4ccc(-c5ccccc5)cc4)cc3)c1n2-c1cccc(-c2nc(-c3ccc(-c4ccccc4)cc3)nc(-c3cccc4oc5ccccc5c34)n2)c1]

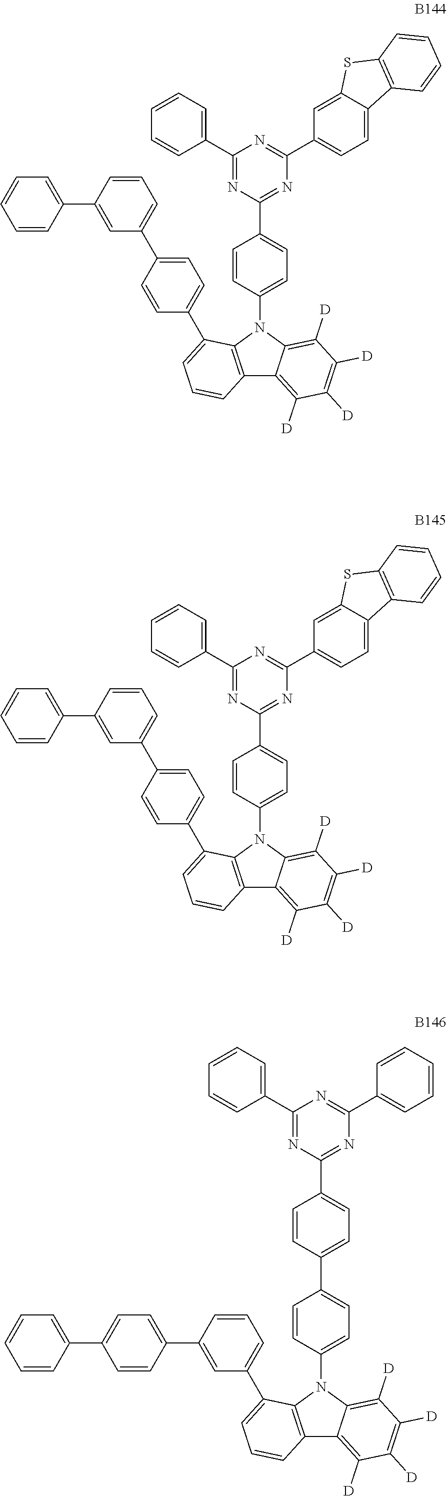 [SMILES: [2H]c1c([2H])c([2H])c2c(c1[2H])c1cccc(-c3ccc(-c4cccc(-c5ccccc5)c4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3ccc(-c4cccc(-c5ccccc5)c4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3cccc(-c4ccc(-c5ccccc5)cc4)c3)c1n2-c1ccc(-c2ccc(-c3nc(-c4ccccc4)nc(-c4ccccc4)n3)cc2)cc1]

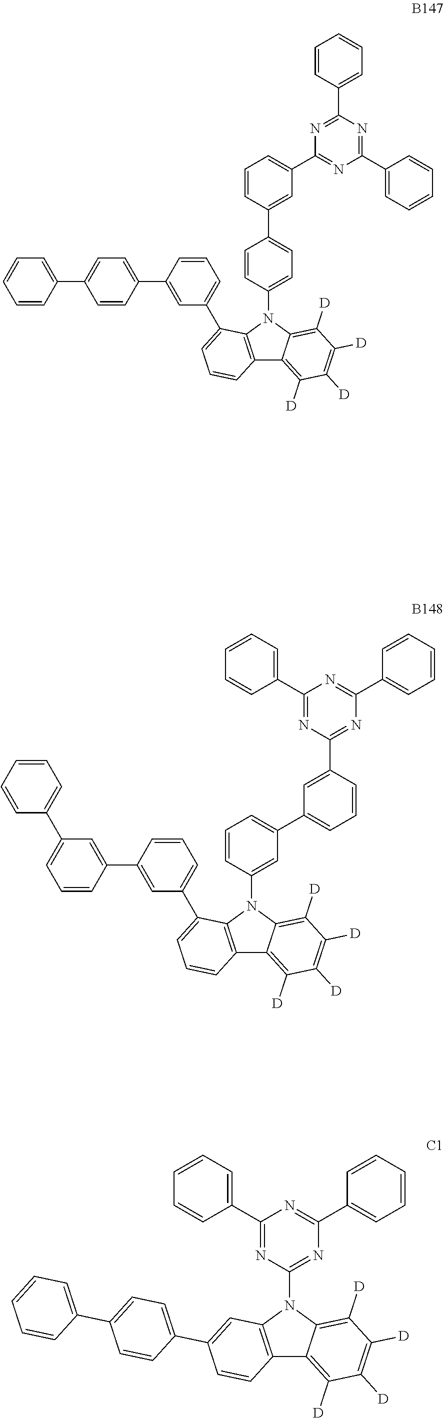 [SMILES: [2H]c1c([2H])c([2H])c2c(c1[2H])c1cccc(-c3cccc(-c4ccc(-c5ccccc5)cc4)c3)c1n2-c1ccc(-c2cccc(-c3nc(-c4ccccc4)nc(-c4ccccc4)n3)c2)cc1 ; [2H]c1c([2H])c([2H])c2c(c1[2H])c1cccc(-c3cccc(-c4cccc(-c5ccccc5)c4)c3)c1n2-c1cccc(-c2cccc(-c3nc(-c4ccccc4)nc(-c4ccccc4)n3)c2)c1 ; [2H]c1c([2H])c([2H])c2c(c1[2H])c1ccc(-c3ccc(-c4ccccc4)cc3)cc1n2-c1nc(-c2ccccc2)nc(-c2ccccc2)n1]

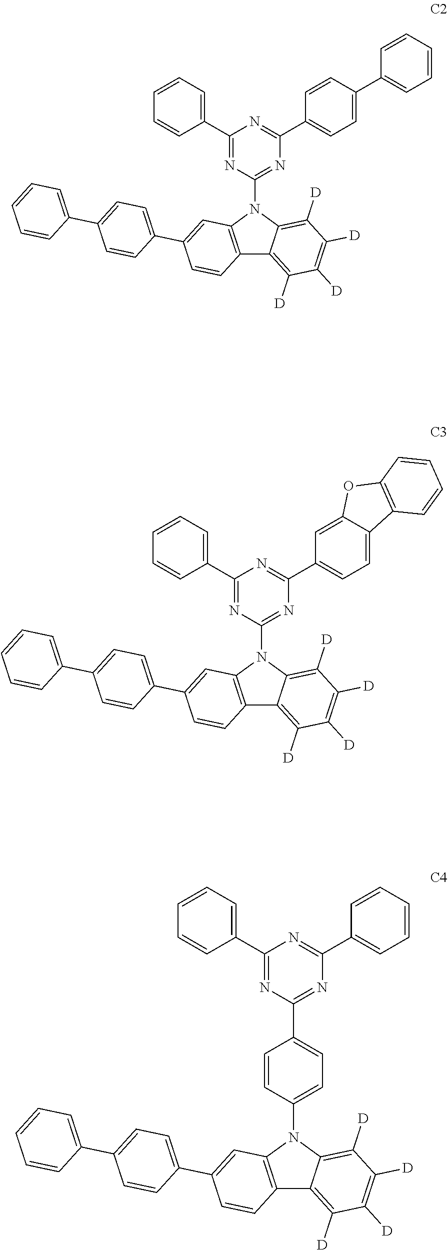 [SMILES: [2H]c1c([2H])c([2H])c2c(c1[2H])c1ccc(-c3ccc(-c4ccccc4)cc3)cc1n2-c1nc(-c2ccccc2)nc(-c2ccc(-c3ccccc3)cc2)n1 ; [2H]c1c([2H])c([2H])c2c(c1[2H])c1ccc(-c3ccc(-c4ccccc4)cc3)cc1n2-c1nc(-c2ccccc2)nc(-c2ccc3c(c2)oc2ccccc23)n1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccccc3)n2)cc1]

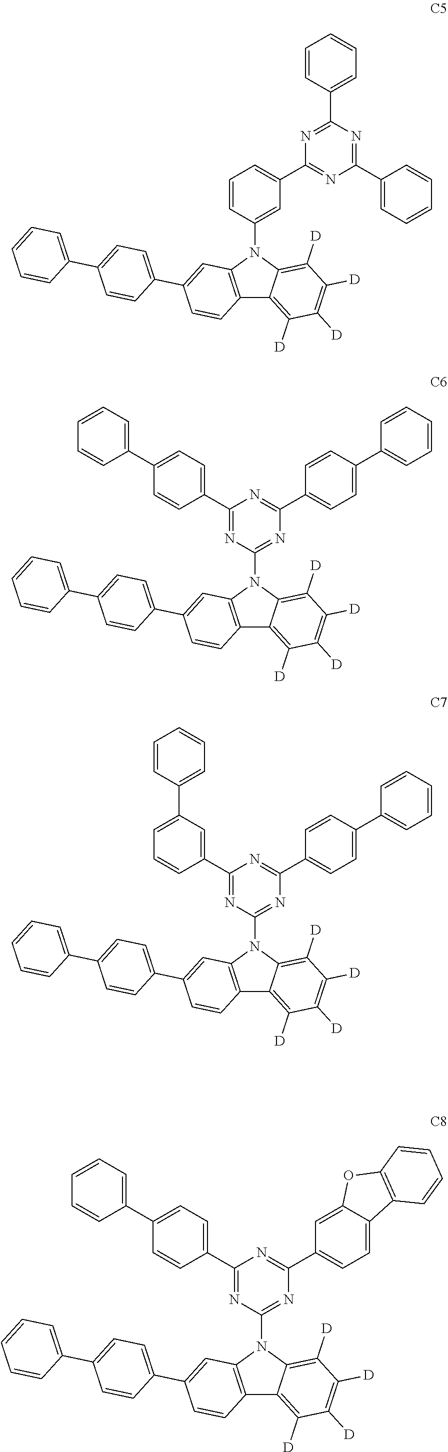 [SMILES: [2H]c1c([2H])c([2H])c2c(c1[2H])c1ccc(-c3ccc(-c4ccccc4)cc3)cc1n2-c1cccc(-c2nc(-c3ccccc3)nc(-c3ccccc3)n2)c1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2ccc(-c3ccccc3)cc2)n1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2cccc(-c3ccccc3)c2)n1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2ccc3c(c2)oc2ccccc23)n1]

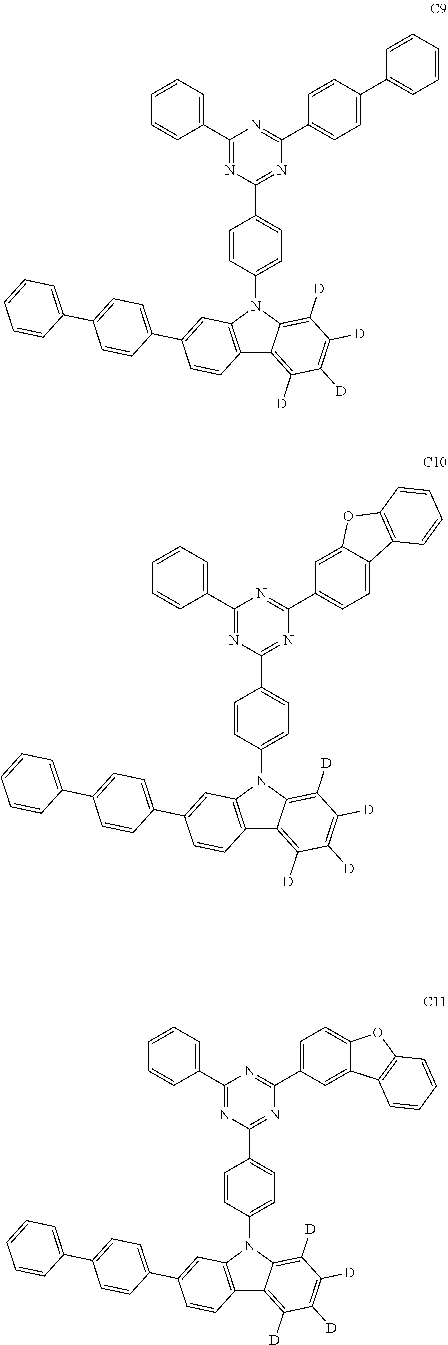 [SMILES: [2H]c1c([2H])c([2H])c2c(c1[2H])c1ccc(-c3ccc(-c4ccccc4)cc3)cc1n2-c1ccc(-c2nc(-c3ccccc3)nc(-c3ccc(-c4ccccc4)cc3)n2)cc1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccc4c(c3)oc3ccccc34)n2)cc1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccc4oc5ccccc5c4c3)n2)cc1]

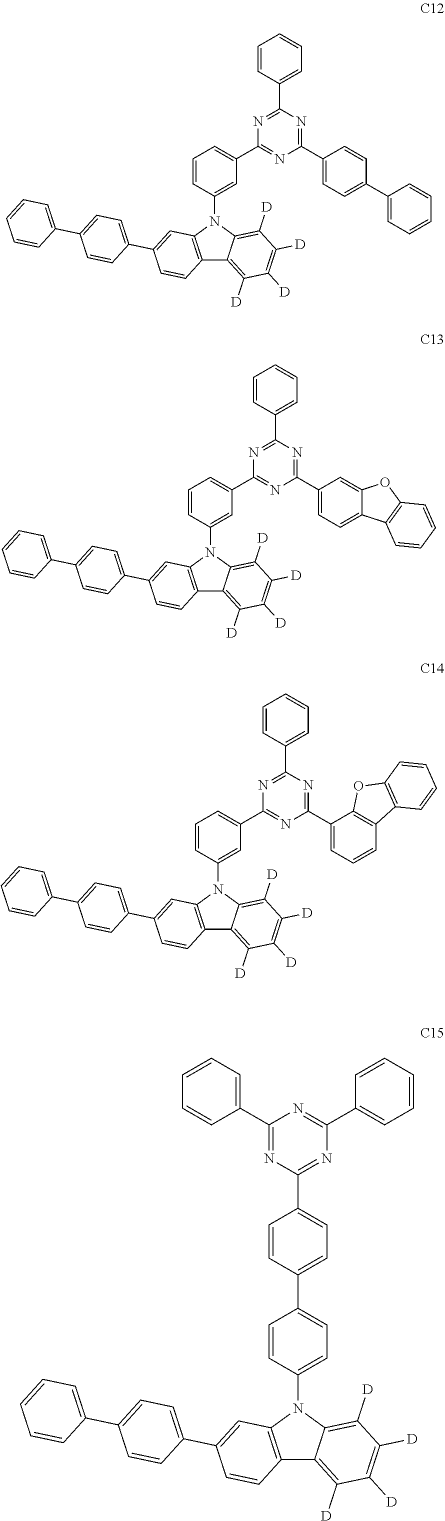 [SMILES: [2H]c1c([2H])c([2H])c2c(c1[2H])c1ccc(-c3ccc(-c4ccccc4)cc3)cc1n2-c1cccc(-c2nc(-c3ccccc3)nc(-c3ccc(-c4ccccc4)cc3)n2)c1 ; [2H]c1c([2H])c([2H])c2c(c1[2H])c1ccc(-c3ccc(-c4ccccc4)cc3)cc1n2-c1cccc(-c2nc(-c3ccccc3)nc(-c3ccc4c(c3)oc3ccccc34)n2)c1 ; [2H]c1c([2H])c([2H])c2c(c1[2H])c1ccc(-c3ccc(-c4ccccc4)cc3)cc1n2-c1cccc(-c2nc(-c3ccccc3)nc(-c3cccc4c3oc3ccccc34)n2)c1 ; [2H]c1c([2H])c([2H])c2c(c1[2H])c1ccc(-c3ccc(-c4ccccc4)cc3)cc1n2-c1ccc(-c2ccc(-c3nc(-c4ccccc4)nc(-c4ccccc4)n3)cc2)cc1]

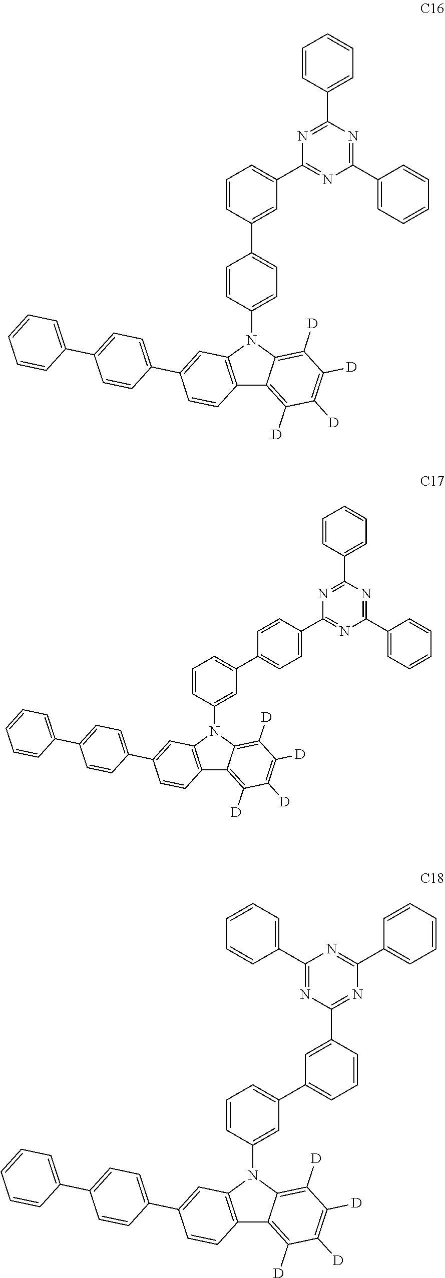 [SMILES: [2H]c1c([2H])c([2H])c2c(c1[2H])c1ccc(-c3ccc(-c4ccccc4)cc3)cc1n2-c1ccc(-c2cccc(-c3nc(-c4ccccc4)nc(-c4ccccc4)n3)c2)cc1 ; [2H]c1c([2H])c([2H])c2c(c1[2H])c1ccc(-c3ccc(-c4ccccc4)cc3)cc1n2-c1cccc(-c2ccc(-c3nc(-c4ccccc4)nc(-c4ccccc4)n3)cc2)c1 ; [2H]c1c([2H])c([2H])c2c(c1[2H])c1ccc(-c3ccc(-c4ccccc4)cc3)cc1n2-c1cccc(-c2cccc(-c3nc(-c4ccccc4)nc(-c4ccccc4)n3)c2)c1]

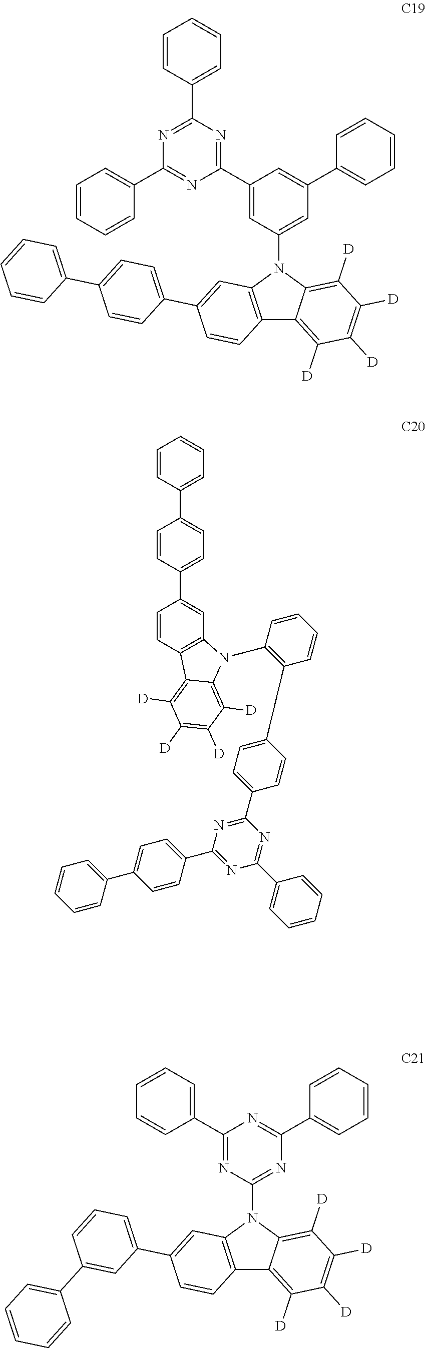 [SMILES: [2H]c1c([2H])c([2H])c2c(c1[2H])c1ccc(-c3ccc(-c4ccccc4)cc3)cc1n2-c1cc(-c2ccccc2)cc(-c2nc(-c3ccccc3)nc(-c3ccccc3)n2)c1 ; [2H]c1c([2H])c([2H])c2c(c1[2H])c1ccc(-c3ccc(-c4ccccc4)cc3)cc1n2-c1ccccc1-c1ccc(-c2nc(-c3ccccc3)nc(-c3ccc(-c4ccccc4)cc3)n2)cc1 ; [2H]c1c([2H])c([2H])c2c(c1[2H])c1ccc(-c3cccc(-c4ccccc4)c3)cc1n2-c1nc(-c2ccccc2)nc(-c2ccccc2)n1]

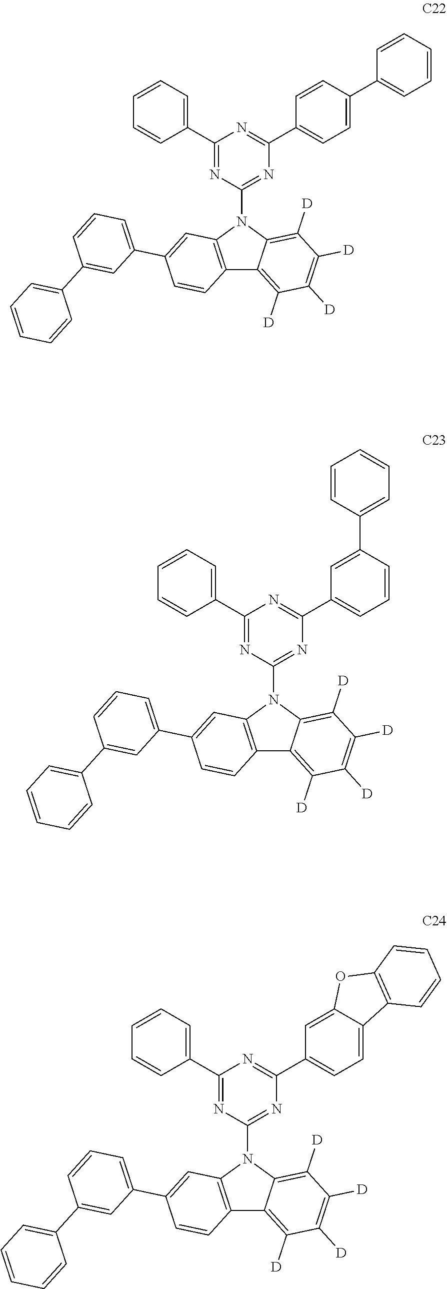 [SMILES: [2H]c1c([2H])c([2H])c2c(c1[2H])c1ccc(-c3cccc(-c4ccccc4)c3)cc1n2-c1nc(-c2ccccc2)nc(-c2ccc(-c3ccccc3)cc2)n1 ; [2H]c1c([2H])c([2H])c2c(c1[2H])c1ccc(-c3cccc(-c4ccccc4)c3)cc1n2-c1nc(-c2ccccc2)nc(-c2cccc(-c3ccccc3)c2)n1 ; [2H]c1c([2H])c([2H])c2c(c1[2H])c1ccc(-c3cccc(-c4ccccc4)c3)cc1n2-c1nc(-c2ccccc2)nc(-c2ccc3c(c2)oc2ccccc23)n1]

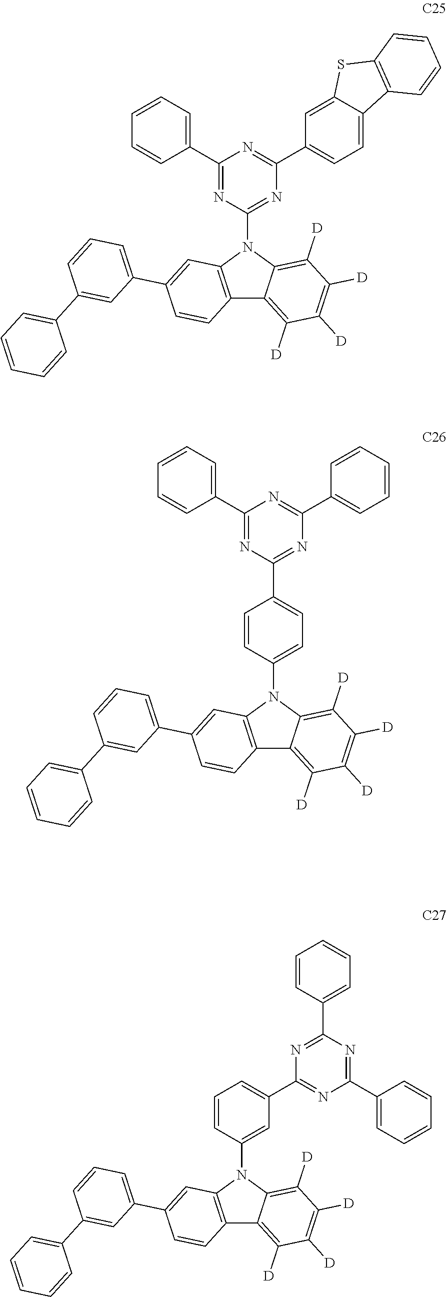 [SMILES: [2H]c1c([2H])c([2H])c2c(c1[2H])c1ccc(-c3cccc(-c4ccccc4)c3)cc1n2-c1nc(-c2ccccc2)nc(-c2ccc3c(c2)sc2ccccc23)n1 ; [2H]c1c([2H])c([2H])c2c(c1[2H])c1ccc(-c3cccc(-c4ccccc4)c3)cc1n2-c1ccc(-c2nc(-c3ccccc3)nc(-c3ccccc3)n2)cc1 ; [2H]c1c([2H])c([2H])c2c(c1[2H])c1ccc(-c3cccc(-c4ccccc4)c3)cc1n2-c1cccc(-c2nc(-c3ccccc3)nc(-c3ccccc3)n2)c1]

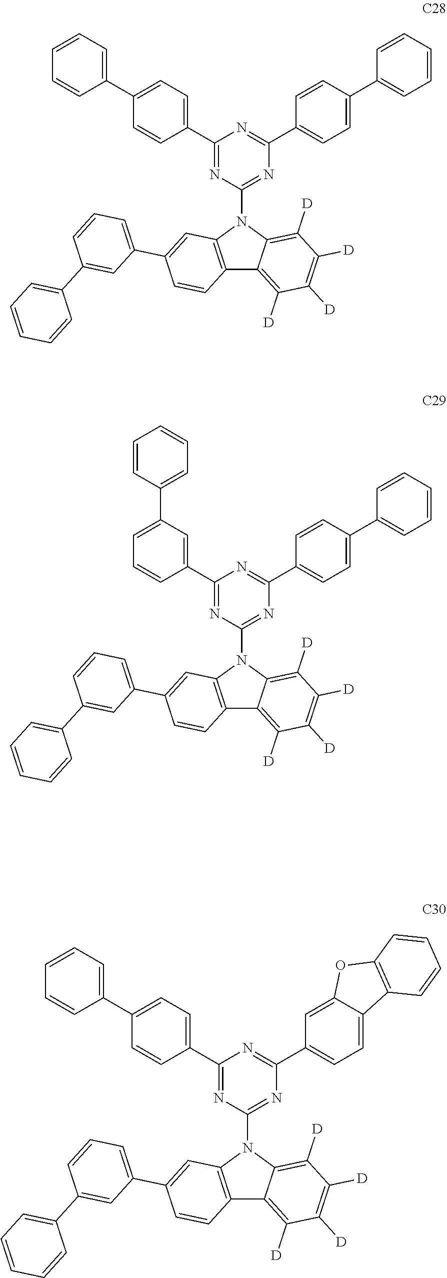 [SMILES: [2H]c1c([2H])c([2H])c2c(c1[2H])c1ccc(-c3cccc(-c4ccccc4)c3)cc1n2-c1nc(-c2ccc(-c3ccccc3)cc2)nc(-c2ccc(-c3ccccc3)cc2)n1 ; [2H]c1c([2H])c([2H])c2c(c1[2H])c1ccc(-c3cccc(-c4ccccc4)c3)cc1n2-c1nc(-c2ccc(-c3ccccc3)cc2)nc(-c2cccc(-c3ccccc3)c2)n1 ; [2H]c1c([2H])c([2H])c2c(c1[2H])c1ccc(-c3cccc(-c4ccccc4)c3)cc1n2-c1nc(-c2ccc(-c3ccccc3)cc2)nc(-c2ccc3c(c2)oc2ccccc23)n1]

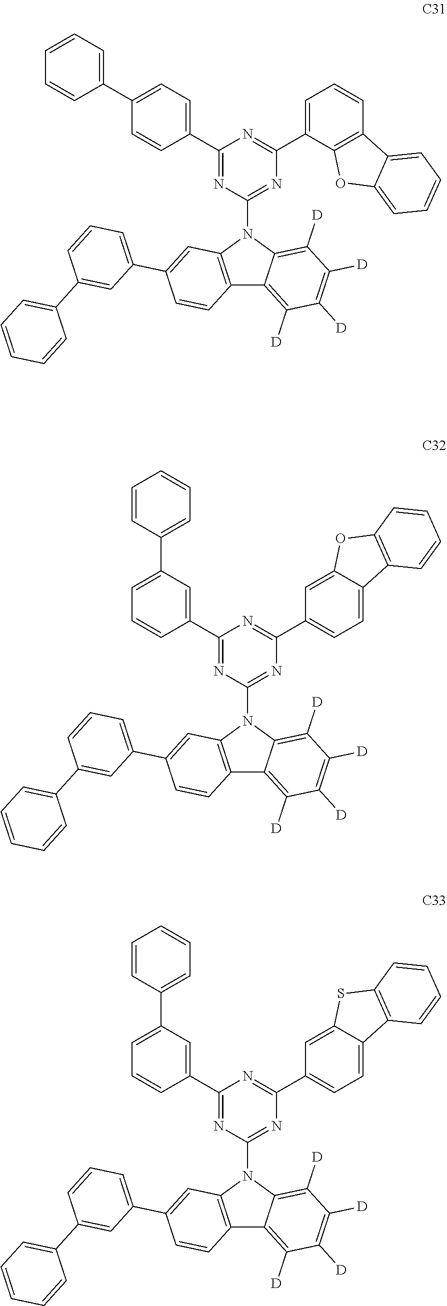 [SMILES: [2H]c1c([2H])c([2H])c2c(c1[2H])c1ccc(-c3cccc(-c4ccccc4)c3)cc1n2-c1nc(-c2ccc(-c3ccccc3)cc2)nc(-c2cccc3c2oc2ccccc23)n1 ; [2H]c1c([2H])c([2H])c2c(c1[2H])c1ccc(-c3cccc(-c4ccccc4)c3)cc1n2-c1nc(-c2cccc(-c3ccccc3)c2)nc(-c2ccc3c(c2)oc2ccccc23)n1 ; [2H]c1c([2H])c([2H])c2c(c1[2H])c1ccc(-c3cccc(-c4ccccc4)c3)cc1n2-c1nc(-c2cccc(-c3ccccc3)c2)nc(-c2ccc3c(c2)sc2ccccc23)n1]

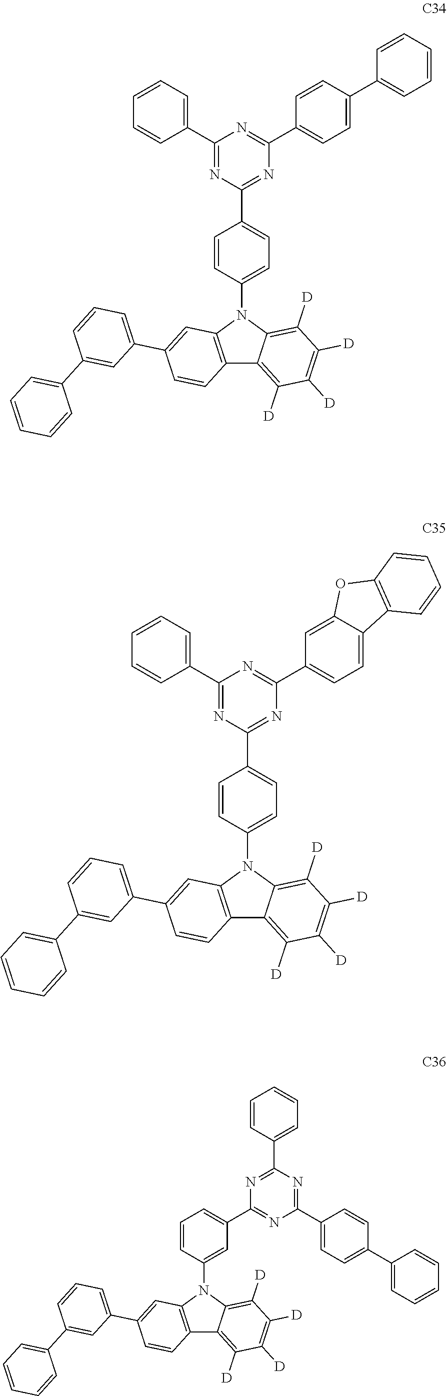 [SMILES: [2H]c1c([2H])c([2H])c2c(c1[2H])c1ccc(-c3cccc(-c4ccccc4)c3)cc1n2-c1ccc(-c2nc(-c3ccccc3)nc(-c3ccc(-c4ccccc4)cc3)n2)cc1 ; [2H]c1c([2H])c([2H])c2c(c1[2H])c1ccc(-c3cccc(-c4ccccc4)c3)cc1n2-c1ccc(-c2nc(-c3ccccc3)nc(-c3ccc4c(c3)oc3ccccc34)n2)cc1 ; [2H]c1c([2H])c([2H])c2c(c1[2H])c1ccc(-c3cccc(-c4ccccc4)c3)cc1n2-c1cccc(-c2nc(-c3ccccc3)nc(-c3ccc(-c4ccccc4)cc3)n2)c1]

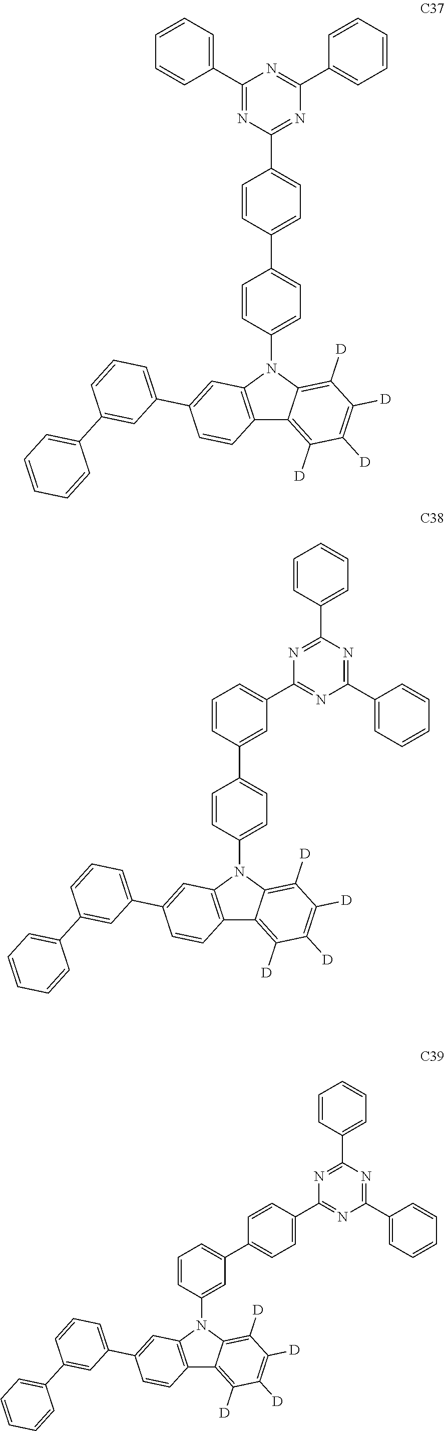 [SMILES: [2H]c1c([2H])c([2H])c2c(c1[2H])c1ccc(-c3cccc(-c4ccccc4)c3)cc1n2-c1ccc(-c2ccc(-c3nc(-c4ccccc4)nc(-c4ccccc4)n3)cc2)cc1 ; [2H]c1c([2H])c([2H])c2c(c1[2H])c1ccc(-c3cccc(-c4ccccc4)c3)cc1n2-c1ccc(-c2cccc(-c3nc(-c4ccccc4)nc(-c4ccccc4)n3)c2)cc1 ; [2H]c1c([2H])c([2H])c2c(c1[2H])c1ccc(-c3cccc(-c4ccccc4)c3)cc1n2-c1cccc(-c2ccc(-c3nc(-c4ccccc4)nc(-c4ccccc4)n3)cc2)c1]

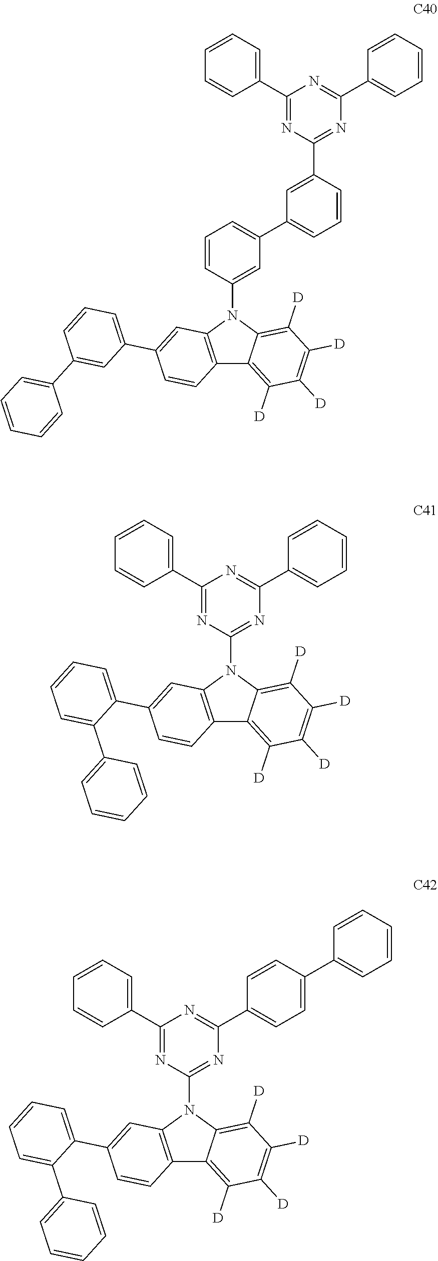 [SMILES: [2H]c1c([2H])c([2H])c2c(c1[2H])c1ccc(-c3cccc(-c4ccccc4)c3)cc1n2-c1cccc(-c2cccc(-c3nc(-c4ccccc4)nc(-c4ccccc4)n3)c2)c1 ; [2H]c1c([2H])c([2H])c2c(c1[2H])c1ccc(-c3ccccc3-c3ccccc3)cc1n2-c1nc(-c2ccccc2)nc(-c2ccccc2)n1 ; [2H]c1c([2H])c([2H])c2c(c1[2H])c1ccc(-c3ccccc3-c3ccccc3)cc1n2-c1nc(-c2ccccc2)nc(-c2ccc(-c3ccccc3)cc2)n1]

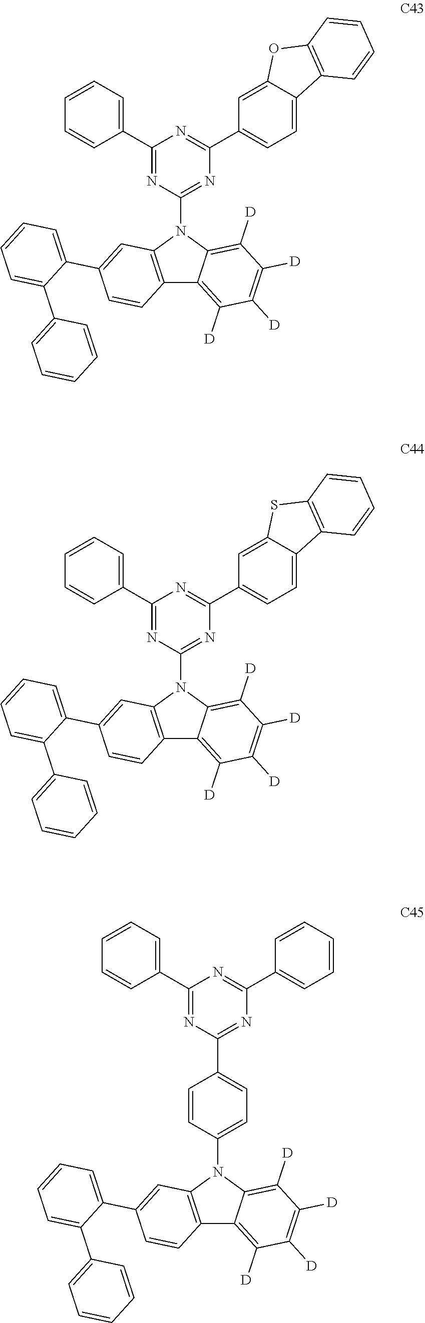 [SMILES: [2H]c1c([2H])c([2H])c2c(c1[2H])c1ccc(-c3ccccc3-c3ccccc3)cc1n2-c1nc(-c2ccccc2)nc(-c2ccc3c(c2)oc2ccccc23)n1 ; [2H]c1c([2H])c([2H])c2c(c1[2H])c1ccc(-c3ccccc3-c3ccccc3)cc1n2-c1nc(-c2ccccc2)nc(-c2ccc3c(c2)sc2ccccc23)n1 ; [2H]c1c([2H])c([2H])c2c(c1[2H])c1ccc(-c3ccccc3-c3ccccc3)cc1n2-c1ccc(-c2nc(-c3ccccc3)nc(-c3ccccc3)n2)cc1]

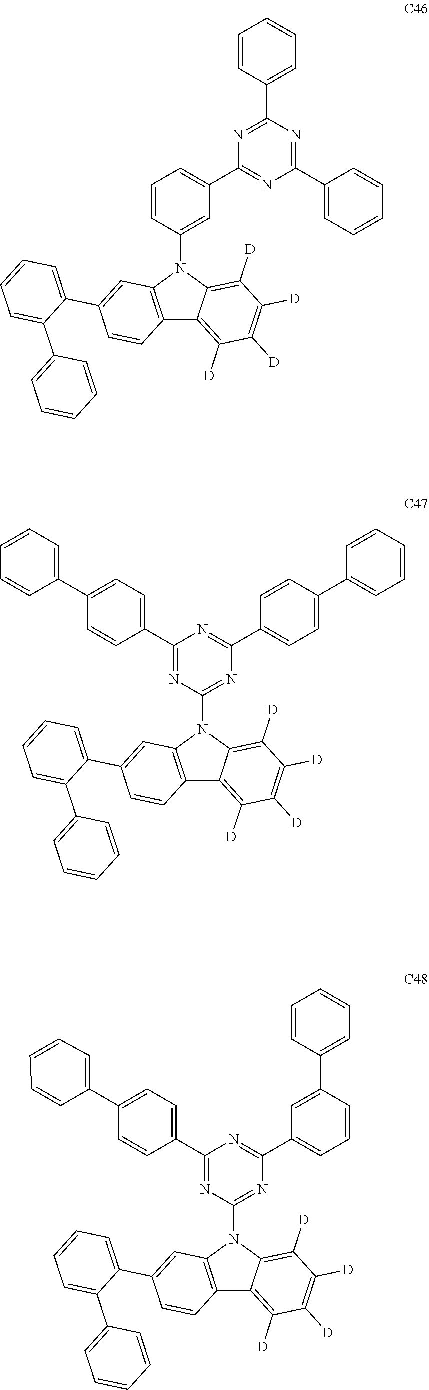 [SMILES: [2H]c1c([2H])c([2H])c2c(c1[2H])c1ccc(-c3ccccc3-c3ccccc3)cc1n2-c1cccc(-c2nc(-c3ccccc3)nc(-c3ccccc3)n2)c1 ; [2H]c1c([2H])c([2H])c2c(c1[2H])c1ccc(-c3ccccc3-c3ccccc3)cc1n2-c1nc(-c2ccc(-c3ccccc3)cc2)nc(-c2ccc(-c3ccccc3)cc2)n1 ; [2H]c1c([2H])c([2H])c2c(c1[2H])c1ccc(-c3ccccc3-c3ccccc3)cc1n2-c1nc(-c2ccc(-c3ccccc3)cc2)nc(-c2cccc(-c3ccccc3)c2)n1]

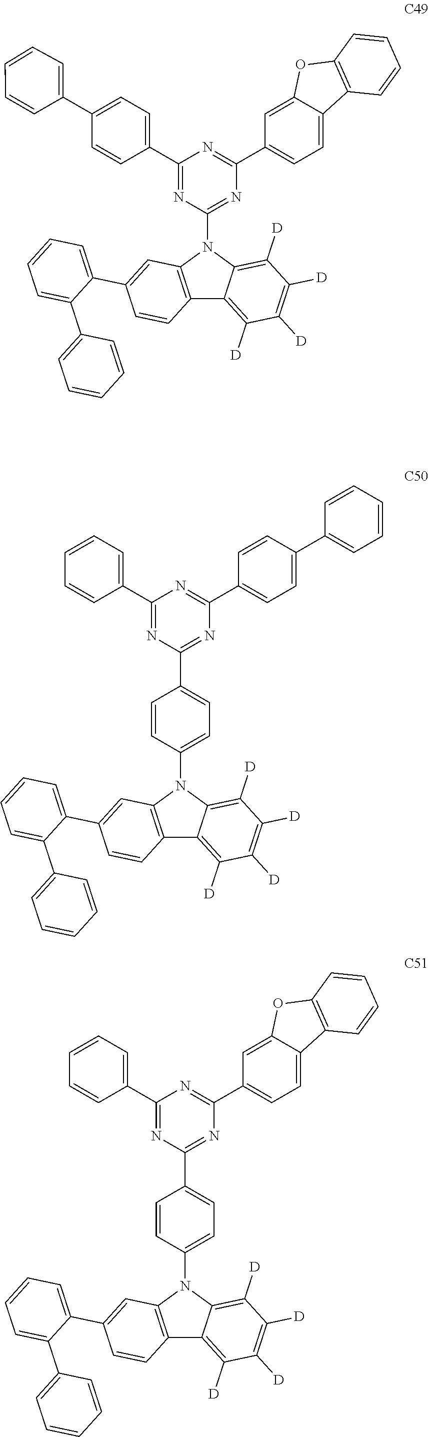 [SMILES: [2H]c1c([2H])c([2H])c2c(c1[2H])c1ccc(-c3ccccc3-c3ccccc3)cc1n2-c1nc(-c2ccc(-c3ccccc3)cc2)nc(-c2ccc3c(c2)oc2ccccc23)n1 ; [2H]c1c([2H])c([2H])c2c(c1[2H])c1ccc(-c3ccccc3-c3ccccc3)cc1n2-c1ccc(-c2nc(-c3ccccc3)nc(-c3ccc(-c4ccccc4)cc3)n2)cc1 ; [2H]c1c([2H])c([2H])c2c(c1[2H])c1ccc(-c3ccccc3-c3ccccc3)cc1n2-c1ccc(-c2nc(-c3ccccc3)nc(-c3ccc4c(c3)oc3ccccc34)n2)cc1]

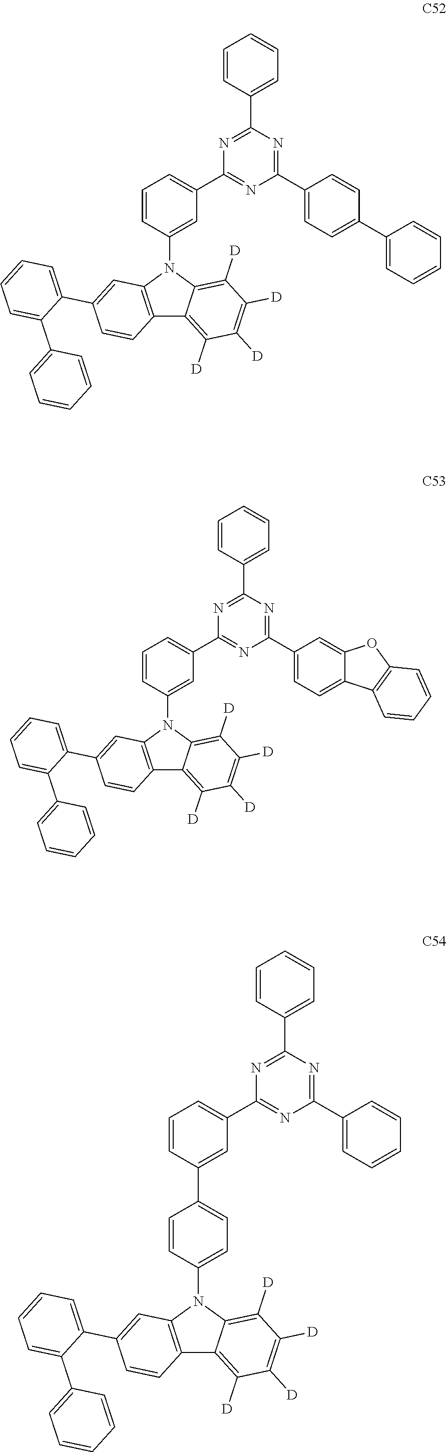 [SMILES: [2H]c1c([2H])c([2H])c2c(c1[2H])c1ccc(-c3ccccc3-c3ccccc3)cc1n2-c1cccc(-c2nc(-c3ccccc3)nc(-c3ccc(-c4ccccc4)cc3)n2)c1 ; [2H]c1c([2H])c([2H])c2c(c1[2H])c1ccc(-c3ccccc3-c3ccccc3)cc1n2-c1cccc(-c2nc(-c3ccccc3)nc(-c3ccc4c(c3)oc3ccccc34)n2)c1 ; [2H]c1c([2H])c([2H])c2c(c1[2H])c1ccc(-c3ccccc3-c3ccccc3)cc1n2-c1ccc(-c2cccc(-c3nc(-c4ccccc4)nc(-c4ccccc4)n3)c2)cc1]

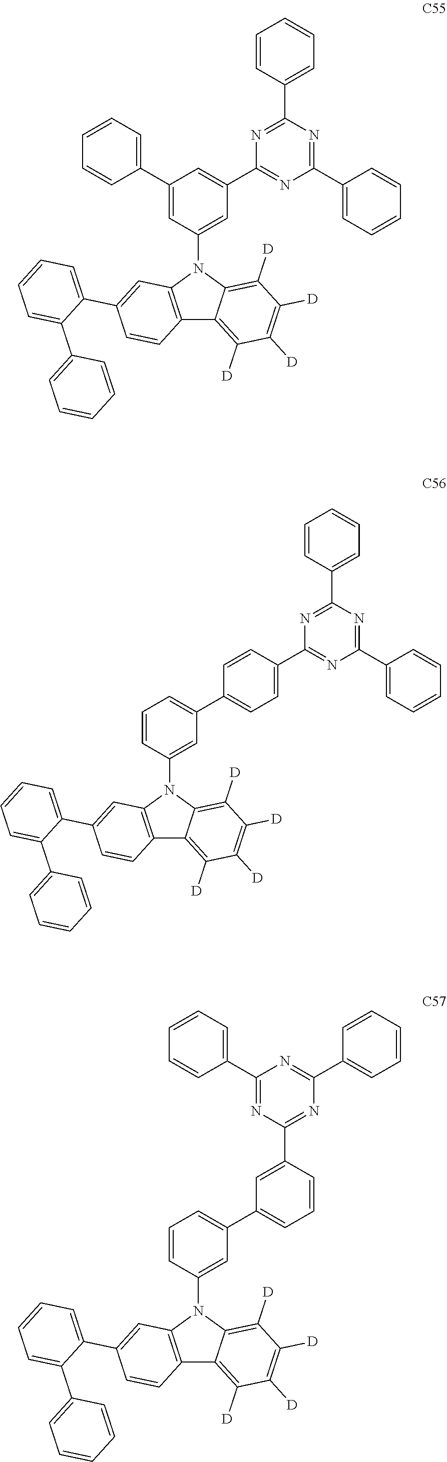 [SMILES: [2H]c1c([2H])c([2H])c2c(c1[2H])c1ccc(-c3ccccc3-c3ccccc3)cc1n2-c1cc(-c2ccccc2)cc(-c2nc(-c3ccccc3)nc(-c3ccccc3)n2)c1 ; [2H]c1c([2H])c([2H])c2c(c1[2H])c1ccc(-c3ccccc3-c3ccccc3)cc1n2-c1cccc(-c2ccc(-c3nc(-c4ccccc4)nc(-c4ccccc4)n3)cc2)c1 ; [2H]c1c([2H])c([2H])c2c(c1[2H])c1ccc(-c3ccccc3-c3ccccc3)cc1n2-c1cccc(-c2cccc(-c3nc(-c4ccccc4)nc(-c4ccccc4)n3)c2)c1]

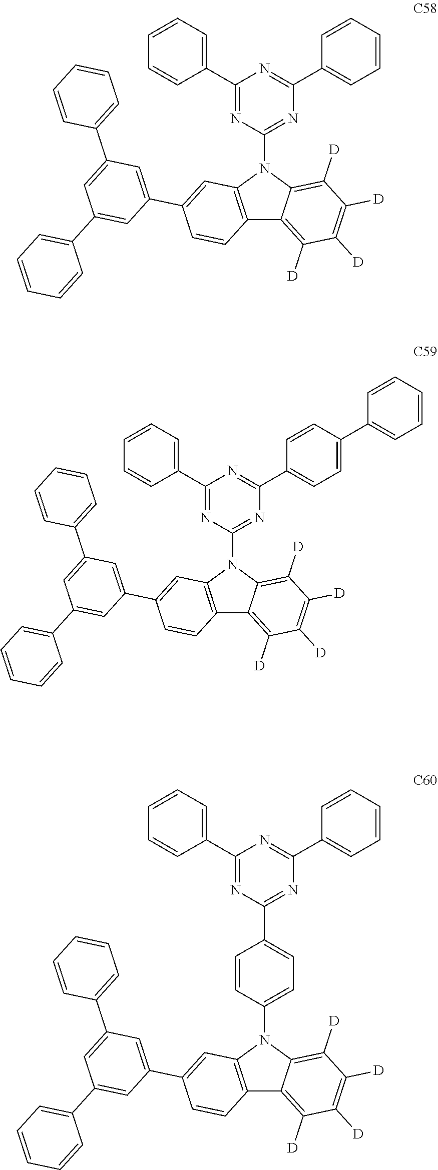

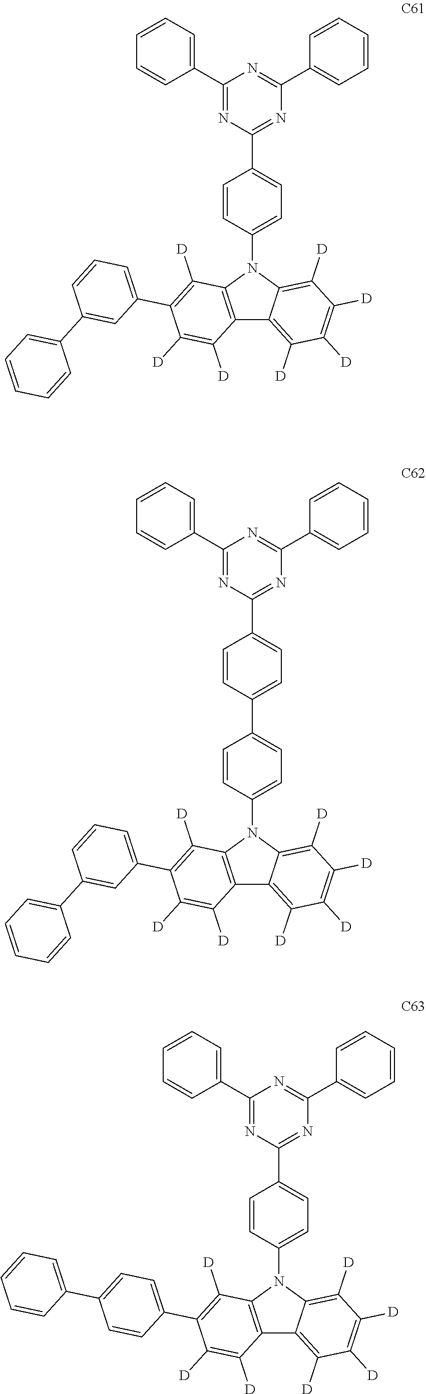

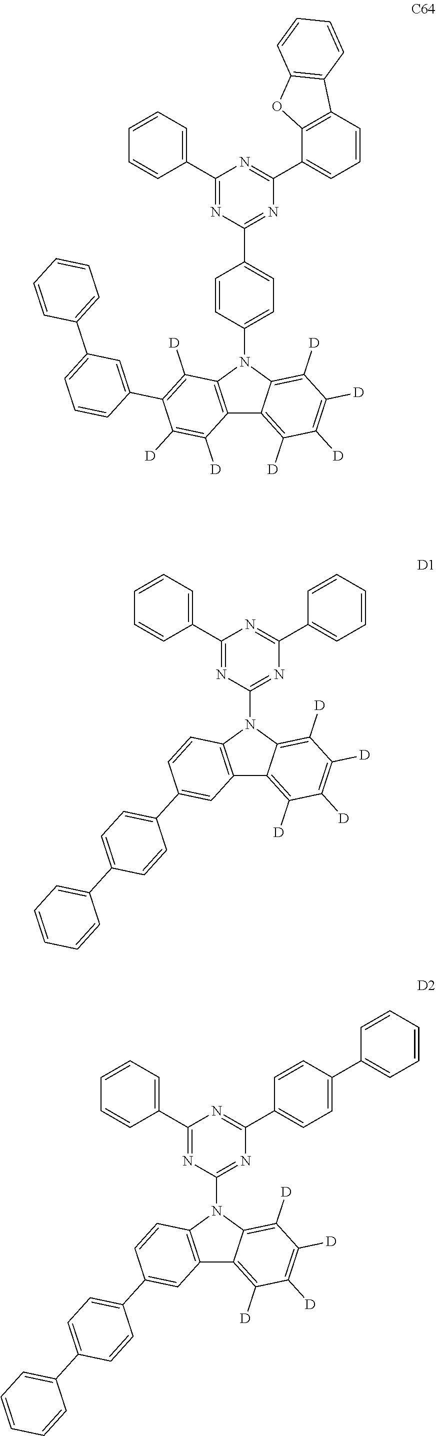

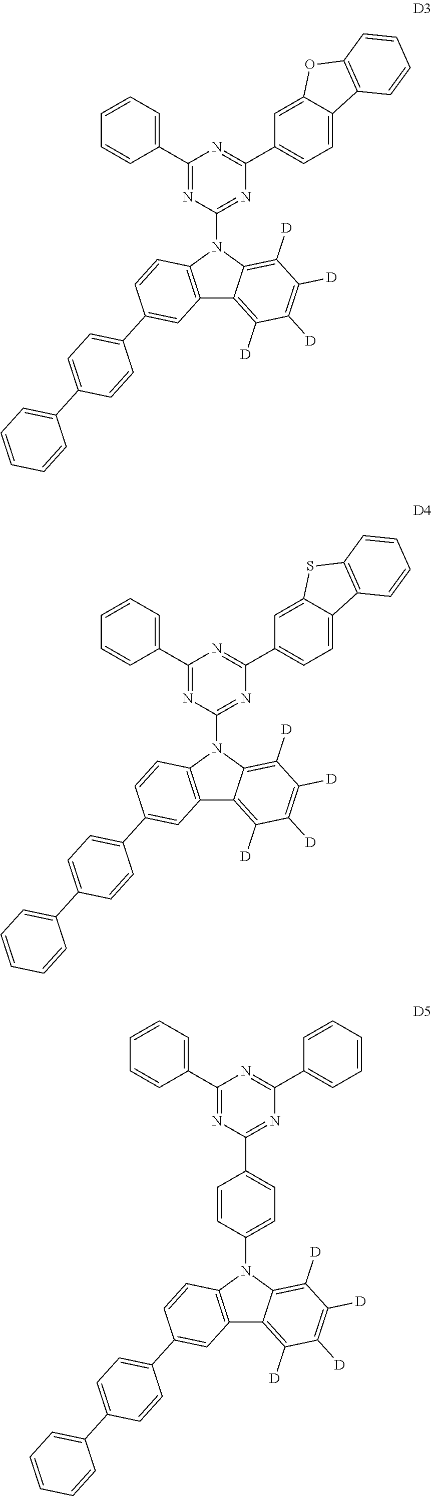 [SMILES: [2H]c1c([2H])c([2H])c2c(c1[2H])c1cc(-c3ccc(-c4ccccc4)cc3)ccc1n2-c1nc(-c2ccccc2)nc(-c2ccc3c(c2)oc2ccccc23)n1 ; [2H]c1c([2H])c([2H])c2c(c1[2H])c1cc(-c3ccc(-c4ccccc4)cc3)ccc1n2-c1nc(-c2ccccc2)nc(-c2ccc3c(c2)sc2ccccc23)n1 ; [2H]c1c([2H])c([2H])c2c(c1[2H])c1cc(-c3ccc(-c4ccccc4)cc3)ccc1n2-c1ccc(-c2nc(-c3ccccc3)nc(-c3ccccc3)n2)cc1]

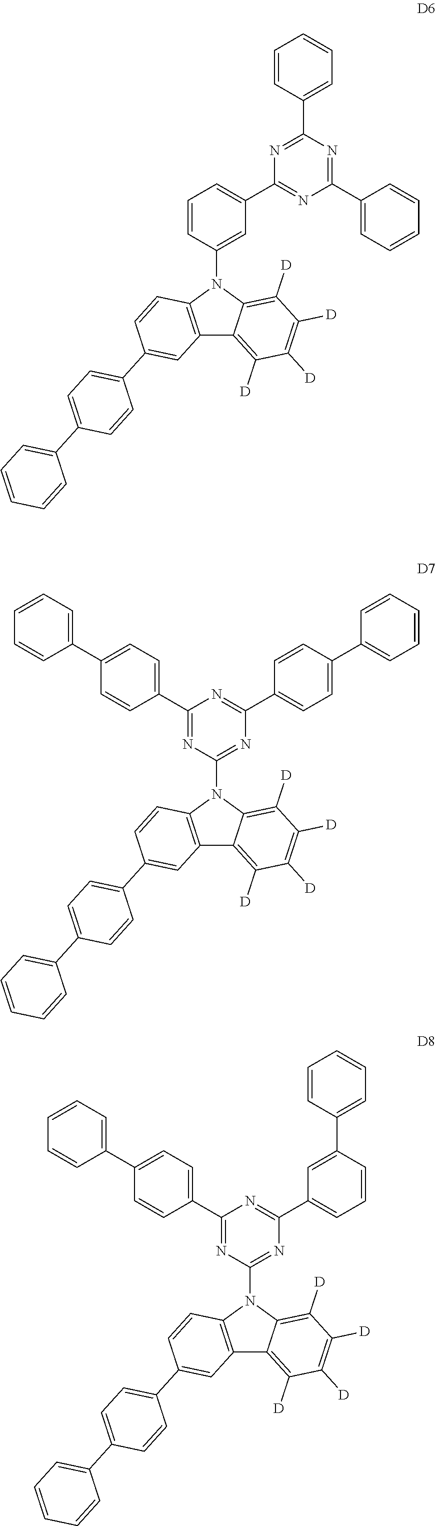 [SMILES: [2H]c1c([2H])c([2H])c2c(c1[2H])c1cc(-c3ccc(-c4ccccc4)cc3)ccc1n2-c1cccc(-c2nc(-c3ccccc3)nc(-c3ccccc3)n2)c1 ; [2H]c1c([2H])c([2H])c2c(c1[2H])c1cc(-c3ccc(-c4ccccc4)cc3)ccc1n2-c1nc(-c2ccc(-c3ccccc3)cc2)nc(-c2ccc(-c3ccccc3)cc2)n1 ; [2H]c1c([2H])c([2H])c2c(c1[2H])c1cc(-c3ccc(-c4ccccc4)cc3)ccc1n2-c1nc(-c2ccc(-c3ccccc3)cc2)nc(-c2cccc(-c3ccccc3)c2)n1]

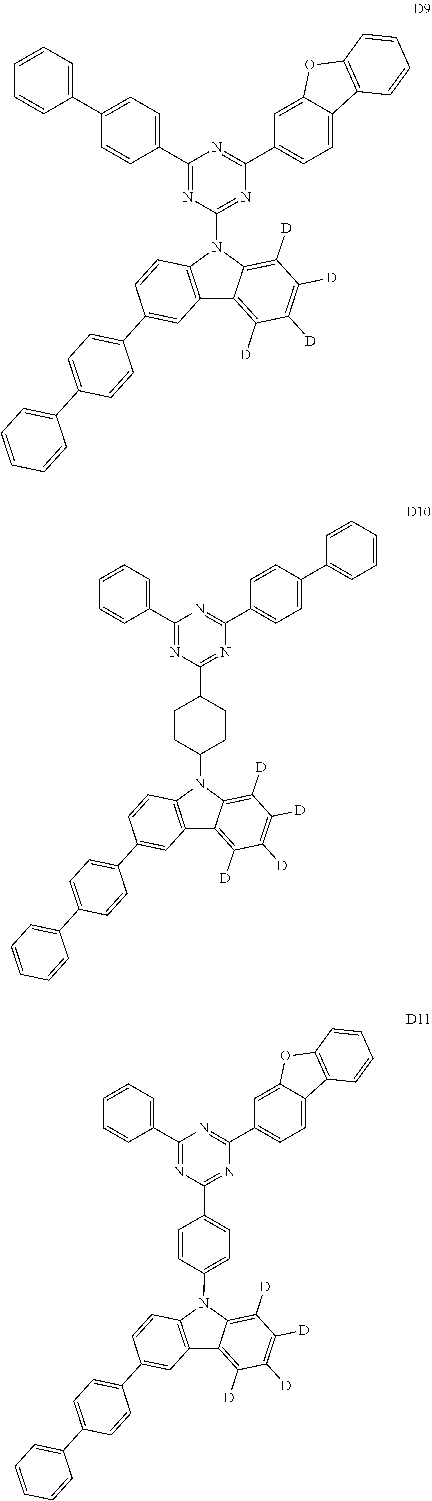 [SMILES: [2H]c1c([2H])c([2H])c2c(c1[2H])c1cc(-c3ccc(-c4ccccc4)cc3)ccc1n2-c1nc(-c2ccc(-c3ccccc3)cc2)nc(-c2ccc3c(c2)oc2ccccc23)n1 ; [2H]c1c([2H])c([2H])c2c(c1[2H])c1cc(-c3ccc(-c4ccccc4)cc3)ccc1n2C1CCC(c2nc(-c3ccccc3)nc(-c3ccc(-c4ccccc4)cc3)n2)CC1 ; [2H]c1c([2H])c([2H])c2c(c1[2H])c1cc(-c3ccc(-c4ccccc4)cc3)ccc1n2-c1ccc(-c2nc(-c3ccccc3)nc(-c3ccc4c(c3)oc3ccccc34)n2)cc1]

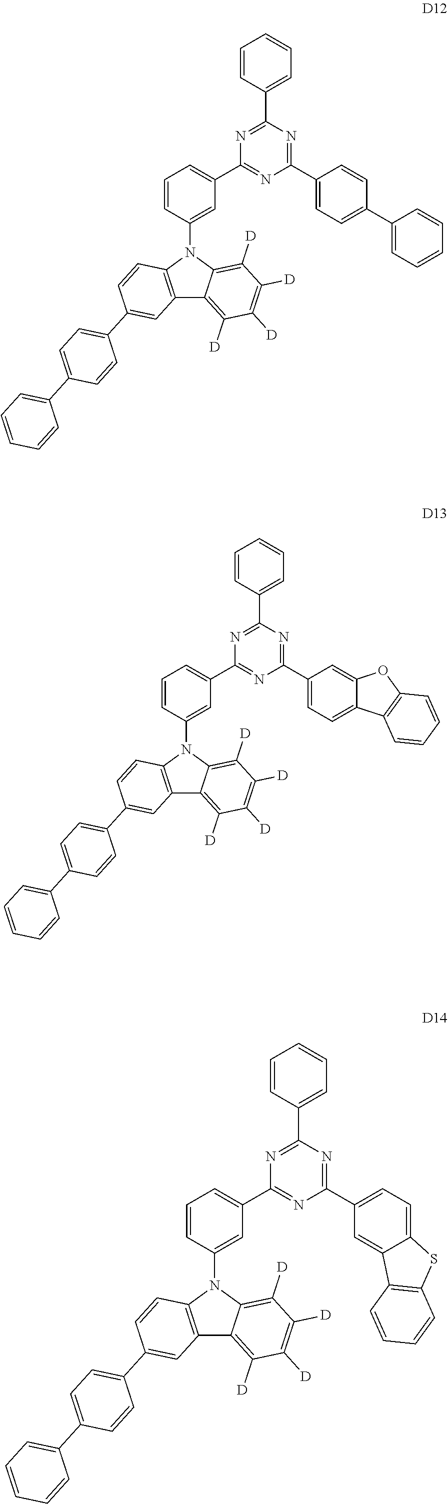 [SMILES: [2H]c1c([2H])c([2H])c2c(c1[2H])c1cc(-c3ccc(-c4ccccc4)cc3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3ccc(-c4ccccc4)cc3)ccc1n2-c1cccc(-c2nc(-c3ccccc3)nc(-c3ccc4c(c3)oc3ccccc34)n2)c1 ; [2H]c1c([2H])c([2H])c2c(c1[2H])c1cc(-c3ccc(-c4ccccc4)cc3)ccc1n2-c1cccc(-c2nc(-c3ccccc3)nc(-c3ccc4sc5ccccc5c4c3)n2)c1]

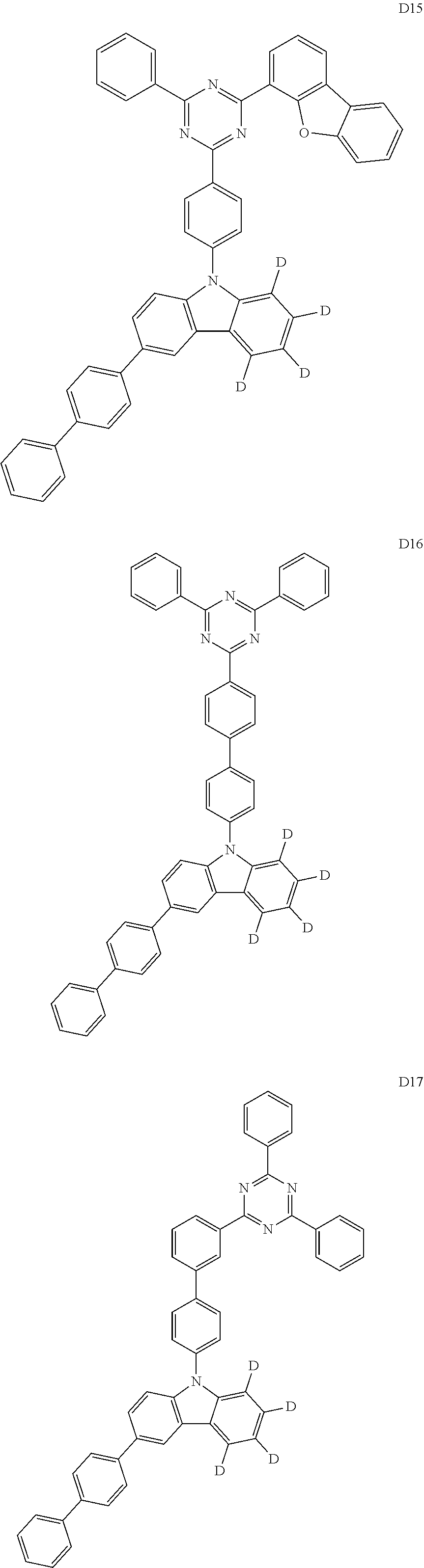 [SMILES: [2H]c1c([2H])c([2H])c2c(c1[2H])c1cc(-c3ccc(-c4ccccc4)cc3)ccc1n2-c1ccc(-c2nc(-c3ccccc3)nc(-c3cccc4c3oc3ccccc34)n2)cc1 ; [2H]c1c([2H])c([2H])c2c(c1[2H])c1cc(-c3ccc(-c4ccccc4)cc3)ccc1n2-c1ccc(-c2ccc(-c3nc(-c4ccccc4)nc(-c4ccccc4)n3)cc2)cc1 ; [2H]c1c([2H])c([2H])c2c(c1[2H])c1cc(-c3ccc(-c4ccccc4)cc3)ccc1n2-c1ccc(-c2cccc(-c3nc(-c4ccccc4)nc(-c4ccccc4)n3)c2)cc1]

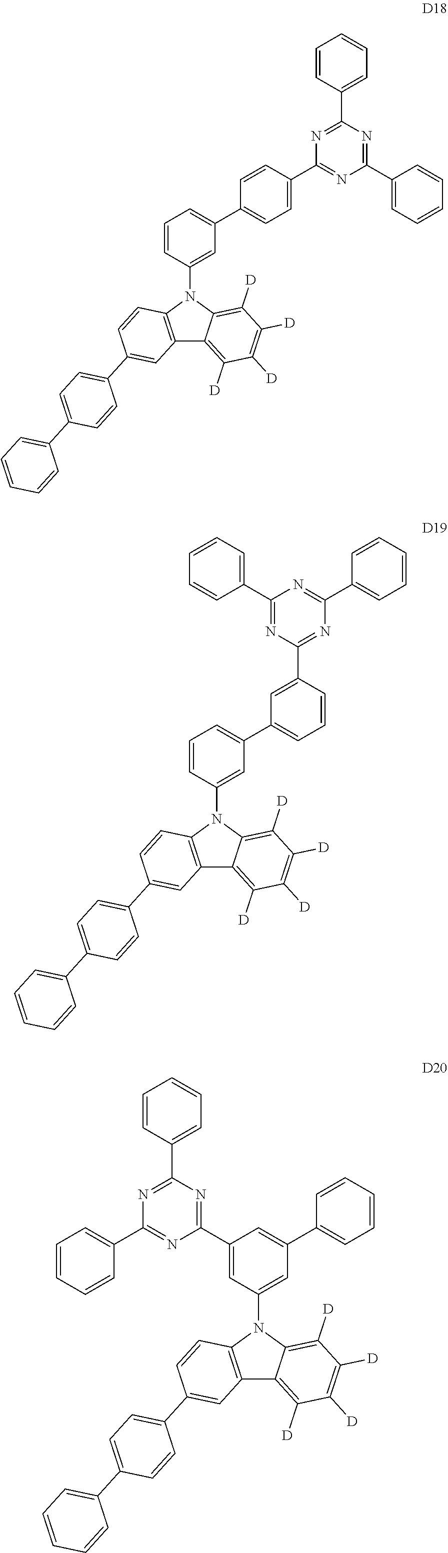 [SMILES: [2H]c1c([2H])c([2H])c2c(c1[2H])c1cc(-c3ccc(-c4ccccc4)cc3)ccc1n2-c1cccc(-c2ccc(-c3nc(-c4ccccc4)nc(-c4ccccc4)n3)cc2)c1 ; [2H]c1c([2H])c([2H])c2c(c1[2H])c1cc(-c3ccc(-c4ccccc4)cc3)ccc1n2-c1cccc(-c2cccc(-c3nc(-c4ccccc4)nc(-c4ccccc4)n3)c2)c1 ; [2H]c1c([2H])c([2H])c2c(c1[2H])c1cc(-c3ccc(-c4ccccc4)cc3)ccc1n2-c1cc(-c2ccccc2)cc(-c2nc(-c3ccccc3)nc(-c3ccccc3)n2)c1]

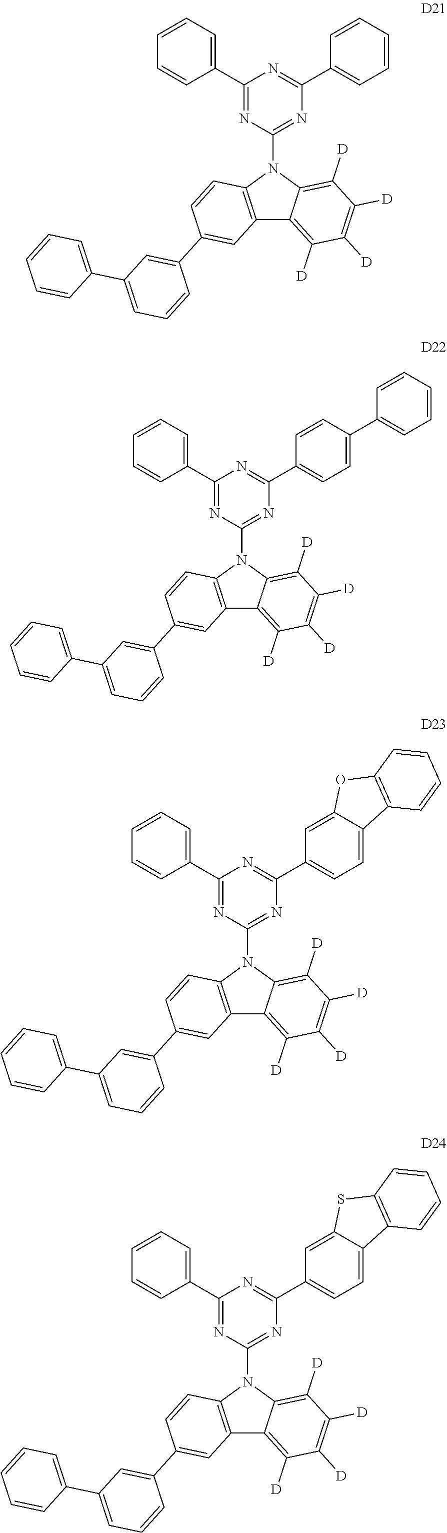 [SMILES: [2H]c1c([2H])c([2H])c2c(c1[2H])c1cc(-c3cccc(-c4ccccc4)c3)ccc1n2-c1nc(-c2ccccc2)nc(-c2ccccc2)n1 ; [2H]c1c([2H])c([2H])c2c(c1[2H])c1cc(-c3cccc(-c4ccccc4)c3)ccc1n2-c1nc(-c2ccccc2)nc(-c2ccc(-c3ccccc3)cc2)n1 ; [2H]c1c([2H])c([2H])c2c(c1[2H])c1cc(-c3cccc(-c4ccccc4)c3)ccc1n2-c1nc(-c2ccccc2)nc(-c2ccc3c(c2)oc2ccccc23)n1 ; [2H]c1c([2H])c([2H])c2c(c1[2H])c1cc(-c3cccc(-c4ccccc4)c3)ccc1n2-c1nc(-c2ccccc2)nc(-c2ccc3c(c2)sc2ccccc23)n1]

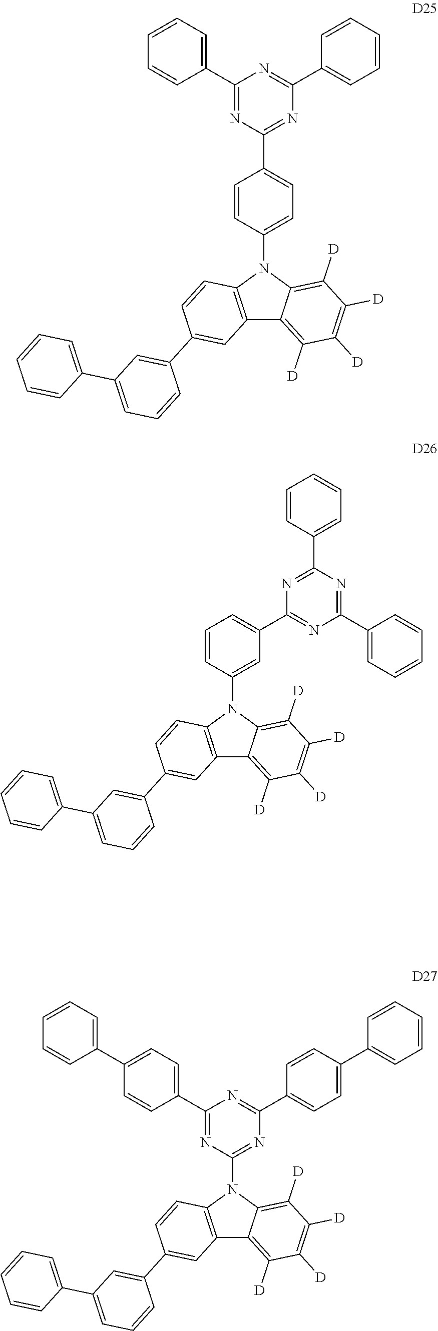 [SMILES: [2H]c1c([2H])c([2H])c2c(c1[2H])c1cc(-c3cccc(-c4ccccc4)c3)ccc1n2-c1ccc(-c2nc(-c3ccccc3)nc(-c3ccccc3)n2)cc1 ; [2H]c1c([2H])c([2H])c2c(c1[2H])c1cc(-c3cccc(-c4ccccc4)c3)ccc1n2-c1cccc(-c2nc(-c3ccccc3)nc(-c3ccccc3)n2)c1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccc(-c3ccccc3)cc2)n1]

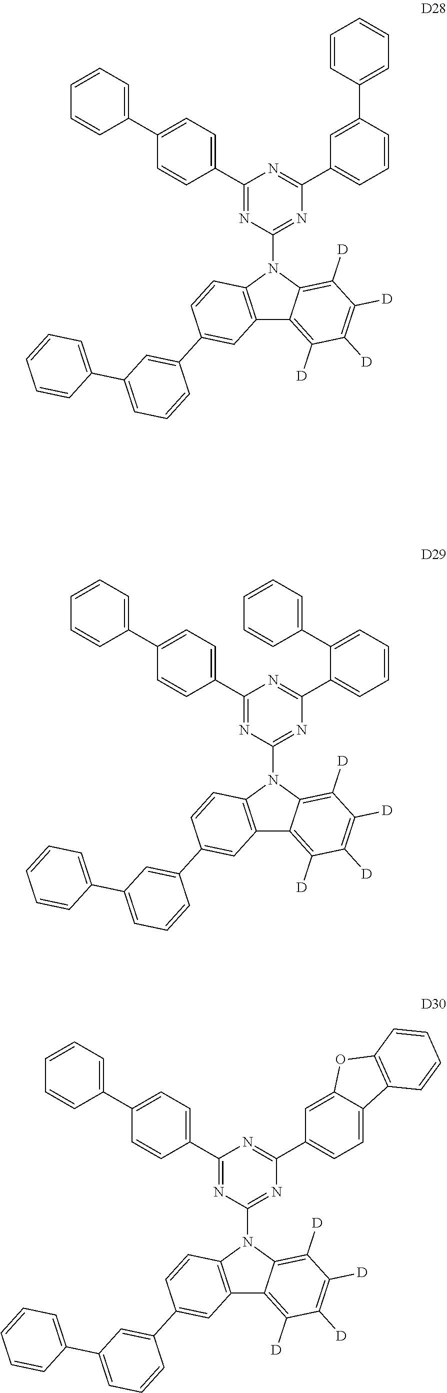 [SMILES: [2H]c1c([2H])c([2H])c2c(c1[2H])c1cc(-c3cccc(-c4ccccc4)c3)ccc1n2-c1nc(-c2ccc(-c3ccccc3)cc2)nc(-c2cccc(-c3ccccc3)c2)n1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccccc2-c2ccccc2)n1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccc3c(c2)oc2ccccc23)n1]

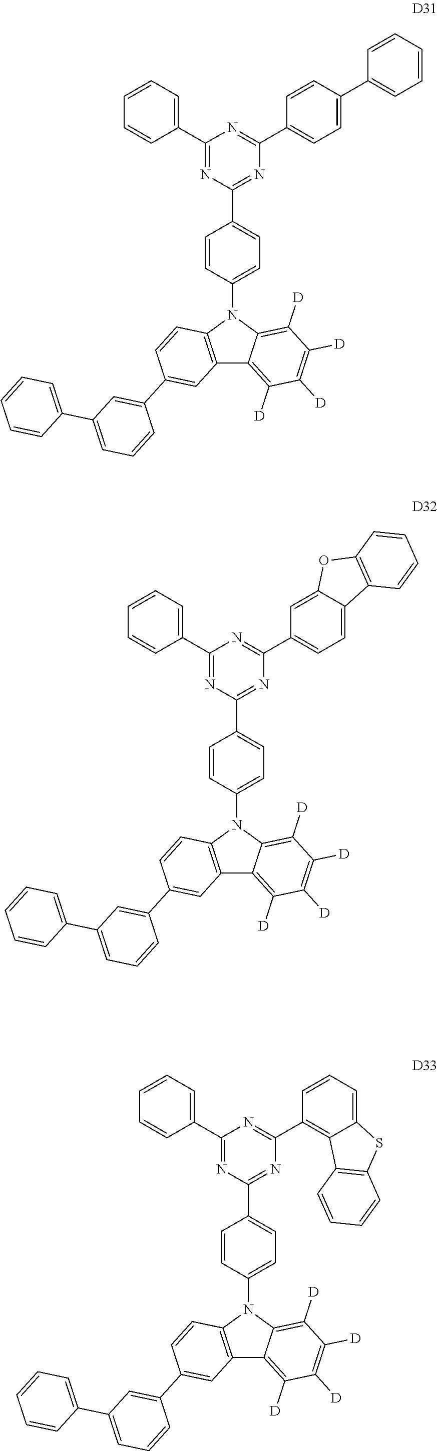 [SMILES: [2H]c1c([2H])c([2H])c2c(c1[2H])c1cc(-c3cccc(-c4ccccc4)c3)ccc1n2-c1ccc(-c2nc(-c3ccccc3)nc(-c3ccc(-c4ccccc4)cc3)n2)cc1 ; [2H]c1c([2H])c([2H])c2c(c1[2H])c1cc(-c3cccc(-c4ccccc4)c3)ccc1n2-c1ccc(-c2nc(-c3ccccc3)nc(-c3ccc4c(c3)oc3ccccc34)n2)cc1 ; [2H]c1c([2H])c([2H])c2c(c1[2H])c1cc(-c3cccc(-c4ccccc4)c3)ccc1n2-c1ccc(-c2nc(-c3ccccc3)nc(-c3cccc4sc5ccccc5c34)n2)cc1]

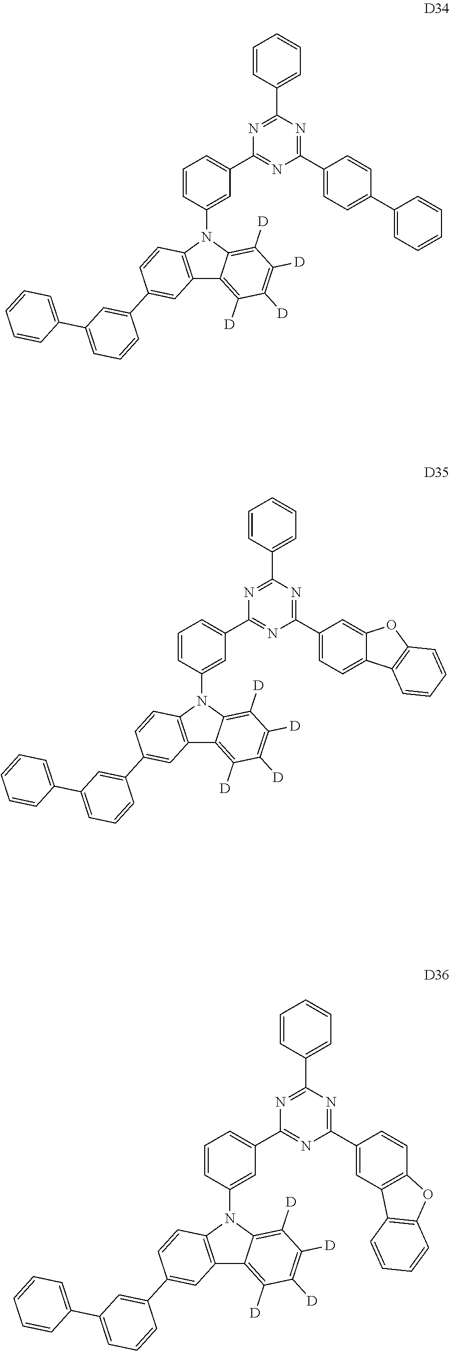 [SMILES: [2H]c1c([2H])c([2H])c2c(c1[2H])c1cc(-c3cccc(-c4ccccc4)c3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3cccc(-c4ccccc4)c3)ccc1n2-c1cccc(-c2nc(-c3ccccc3)nc(-c3ccc4c(c3)oc3ccccc34)n2)c1 ; [2H]c1c([2H])c([2H])c2c(c1[2H])c1cc(-c3cccc(-c4ccccc4)c3)ccc1n2-c1cccc(-c2nc(-c3ccccc3)nc(-c3ccc4oc5ccccc5c4c3)n2)c1]

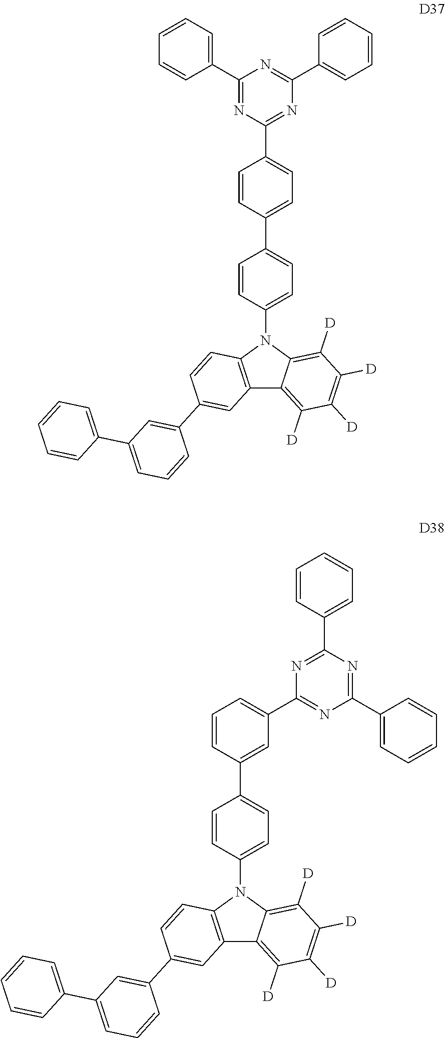 [SMILES: [2H]c1c([2H])c([2H])c2c(c1[2H])c1cc(-c3cccc(-c4ccccc4)c3)ccc1n2-c1ccc(-c2ccc(-c3nc(-c4ccccc4)nc(-c4ccccc4)n3)cc2)cc1 ; [2H]c1c([2H])c([2H])c2c(c1[2H])c1cc(-c3cccc(-c4ccccc4)c3)ccc1n2-c1ccc(-c2cccc(-c3nc(-c4ccccc4)nc(-c4ccccc4)n3)c2)cc1]

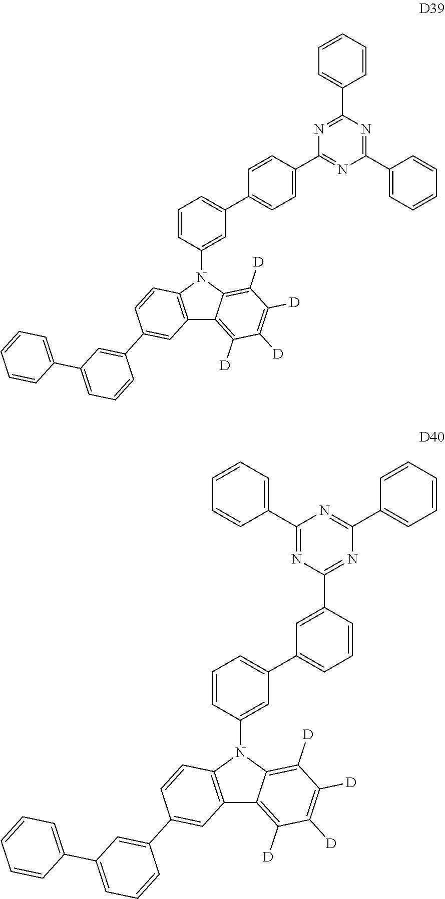 [SMILES: [2H]c1c([2H])c([2H])c2c(c1[2H])c1cc(-c3cccc(-c4ccccc4)c3)ccc1n2-c1cccc(-c2ccc(-c3nc(-c4ccccc4)nc(-c4ccccc4)n3)cc2)c1 ; [2H]c1c([2H])c([2H])c2c(c1[2H])c1cc(-c3cccc(-c4ccccc4)c3)ccc1n2-c1cccc(-c2cccc(-c3nc(-c4ccccc4)nc(-c4ccccc4)n3)c2)c1]

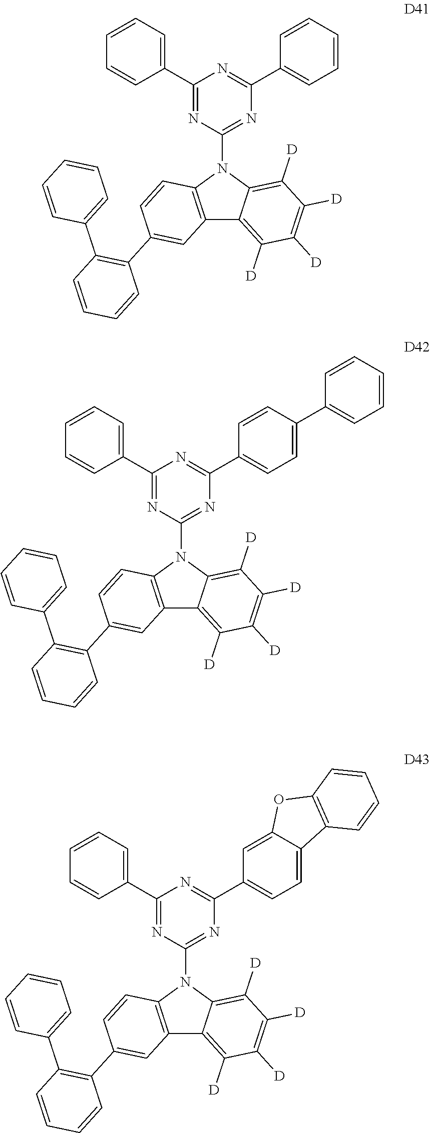 [SMILES: [2H]c1c([2H])c([2H])c2c(c1[2H])c1cc(-c3ccccc3-c3ccccc3)ccc1n2-c1nc(-c2ccccc2)nc(-c2ccccc2)n1 ; [2H]c1c([2H])c([2H])c2c(c1[2H])c1cc(-c3ccccc3-c3ccccc3)ccc1n2-c1nc(-c2ccccc2)nc(-c2ccc(-c3ccccc3)cc2)n1 ; [2H]c1c([2H])c([2H])c2c(c1[2H])c1cc(-c3ccccc3-c3ccccc3)ccc1n2-c1nc(-c2ccccc2)nc(-c2ccc3c(c2)oc2ccccc23)n1]

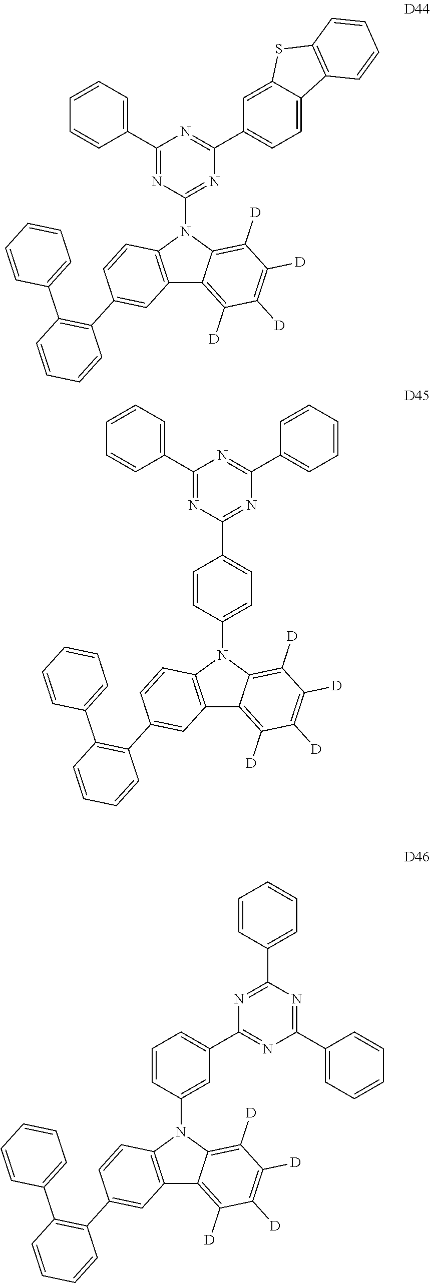 [SMILES: [2H]c1c([2H])c([2H])c2c(c1[2H])c1cc(-c3ccccc3-c3ccccc3)ccc1n2-c1nc(-c2ccccc2)nc(-c2ccc3c(c2)sc2ccccc23)n1 ; [2H]c1c([2H])c([2H])c2c(c1[2H])c1cc(-c3ccccc3-c3ccccc3)ccc1n2-c1ccc(-c2nc(-c3ccccc3)nc(-c3ccccc3)n2)cc1 ; [2H]c1c([2H])c([2H])c2c(c1[2H])c1cc(-c3ccccc3-c3ccccc3)ccc1n2-c1cccc(-c2nc(-c3ccccc3)nc(-c3ccccc3)n2)c1]

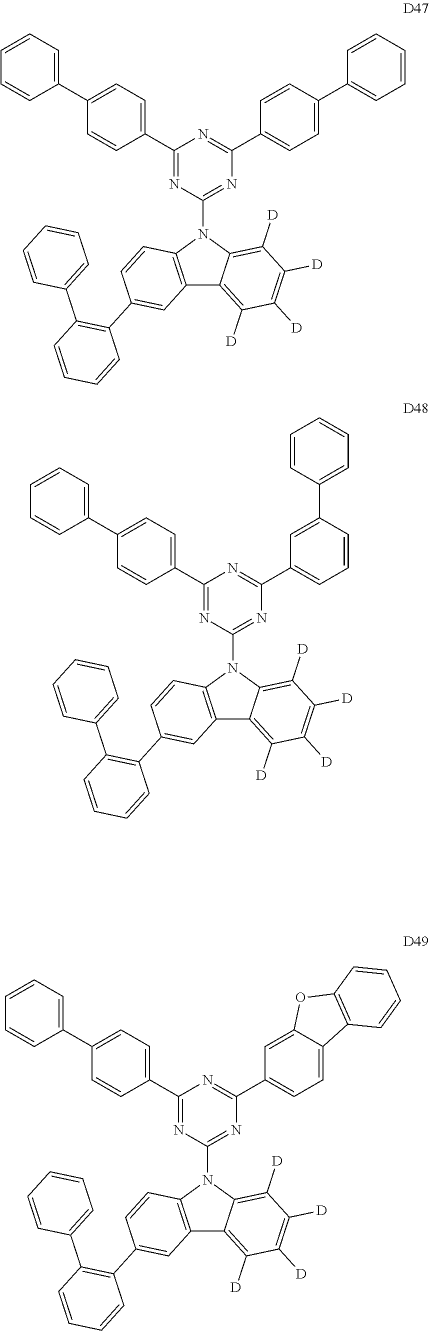 [SMILES: [2H]c1c([2H])c([2H])c2c(c1[2H])c1cc(-c3ccccc3-c3ccccc3)ccc1n2-c1nc(-c2ccc(-c3ccccc3)cc2)nc(-c2ccc(-c3ccccc3)cc2)n1 ; [2H]c1c([2H])c([2H])c2c(c1[2H])c1cc(-c3ccccc3-c3ccccc3)ccc1n2-c1nc(-c2ccc(-c3ccccc3)cc2)nc(-c2cccc(-c3ccccc3)c2)n1 ; [2H]c1c([2H])c([2H])c2c(c1[2H])c1cc(-c3ccccc3-c3ccccc3)ccc1n2-c1nc(-c2ccc(-c3ccccc3)cc2)nc(-c2ccc3c(c2)oc2ccccc23)n1]

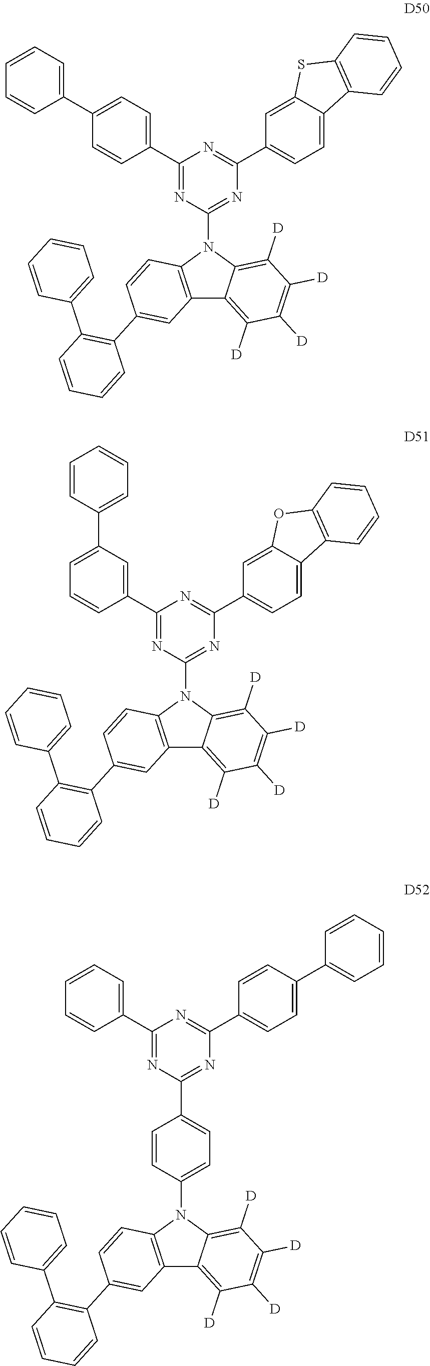 [SMILES: [2H]c1c([2H])c([2H])c2c(c1[2H])c1cc(-c3ccccc3-c3ccccc3)ccc1n2-c1nc(-c2ccc(-c3ccccc3)cc2)nc(-c2ccc3c(c2)sc2ccccc23)n1 ; [2H]c1c([2H])c([2H])c2c(c1[2H])c1cc(-c3ccccc3-c3ccccc3)ccc1n2-c1nc(-c2cccc(-c3ccccc3)c2)nc(-c2ccc3c(c2)oc2ccccc23)n1 ; [2H]c1c([2H])c([2H])c2c(c1[2H])c1cc(-c3ccccc3-c3ccccc3)ccc1n2-c1ccc(-c2nc(-c3ccccc3)nc(-c3ccc(-c4ccccc4)cc3)n2)cc1]

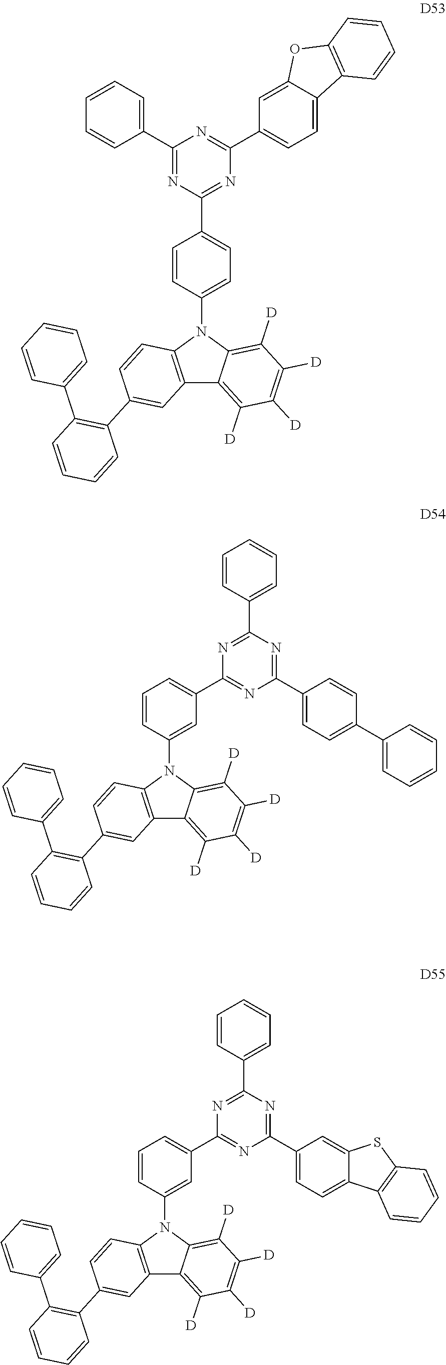 [SMILES: [2H]c1c([2H])c([2H])c2c(c1[2H])c1cc(-c3ccccc3-c3ccccc3)ccc1n2-c1ccc(-c2nc(-c3ccccc3)nc(-c3ccc4c(c3)oc3ccccc34)n2)cc1 ; [2H]c1c([2H])c([2H])c2c(c1[2H])c1cc(-c3ccccc3-c3ccccc3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3ccccc3-c3ccccc3)ccc1n2-c1cccc(-c2nc(-c3ccccc3)nc(-c3ccc4c(c3)sc3ccccc34)n2)c1]

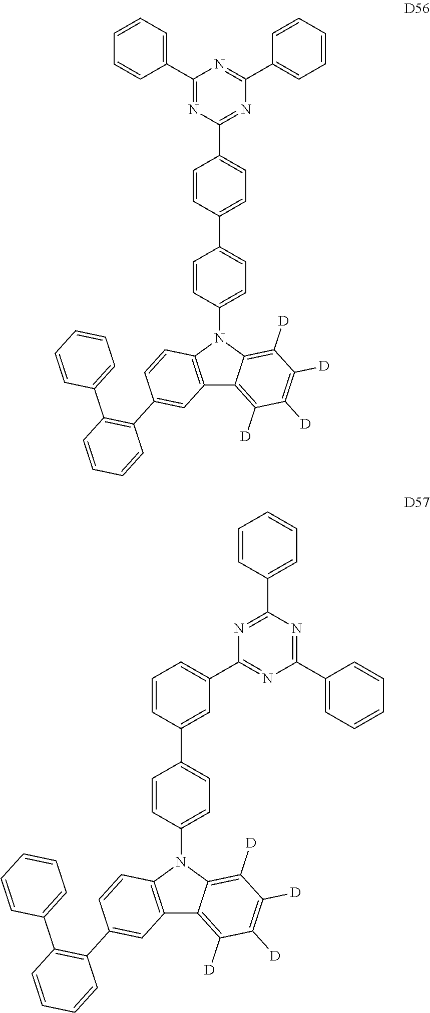 [SMILES: [2H]c1c([2H])c([2H])c2c(c1[2H])c1cc(-c3ccccc3-c3ccccc3)ccc1n2-c1ccc(-c2ccc(-c3nc(-c4ccccc4)nc(-c4ccccc4)n3)cc2)cc1 ; [2H]c1c([2H])c([2H])c2c(c1[2H])c1cc(-c3ccccc3-c3ccccc3)ccc1n2-c1ccc(-c2cccc(-c3nc(-c4ccccc4)nc(-c4ccccc4)n3)c2)cc1]

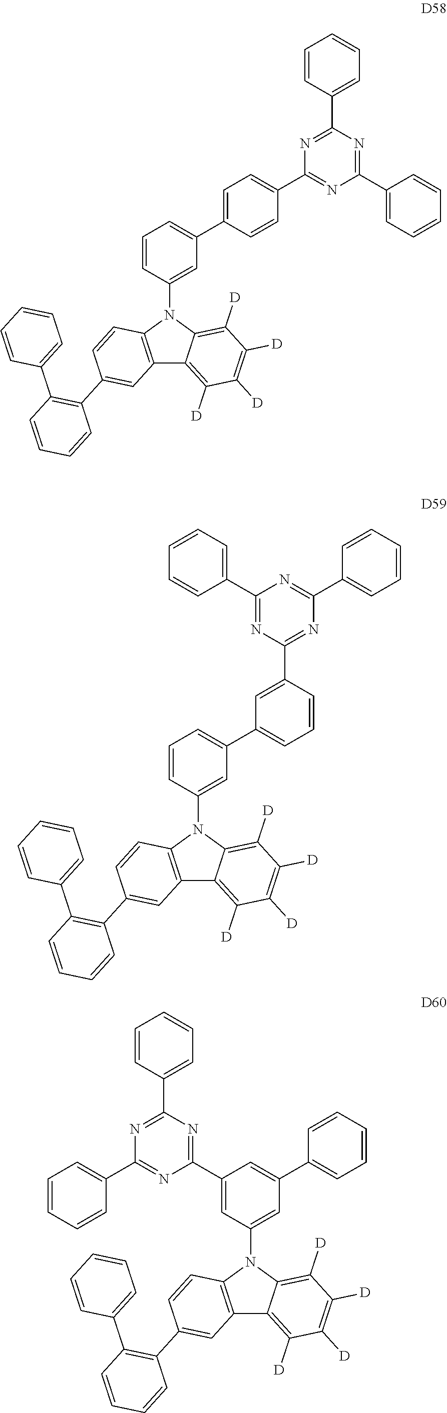 [SMILES: [2H]c1c([2H])c([2H])c2c(c1[2H])c1cc(-c3ccccc3-c3ccccc3)ccc1n2-c1cccc(-c2ccc(-c3nc(-c4ccccc4)nc(-c4ccccc4)n3)cc2)c1 ; [2H]c1c([2H])c([2H])c2c(c1[2H])c1cc(-c3ccccc3-c3ccccc3)ccc1n2-c1cccc(-c2cccc(-c3nc(-c4ccccc4)nc(-c4ccccc4)n3)c2)c1 ; [2H]c1c([2H])c([2H])c2c(c1[2H])c1cc(-c3ccccc3-c3ccccc3)ccc1n2-c1cc(-c2ccccc2)cc(-c2nc(-c3ccccc3)nc(-c3ccccc3)n2)c1]

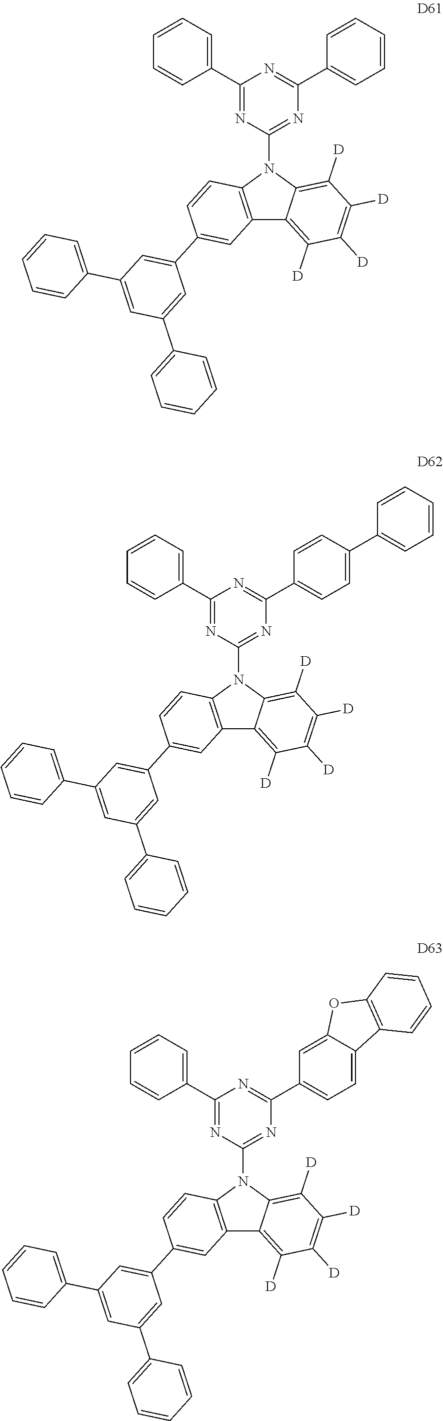 [SMILES: [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccccc2)n1 ; [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccc(-c3ccccc3)cc2)n1 ; [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccc3c(c2)oc2ccccc23)n1]

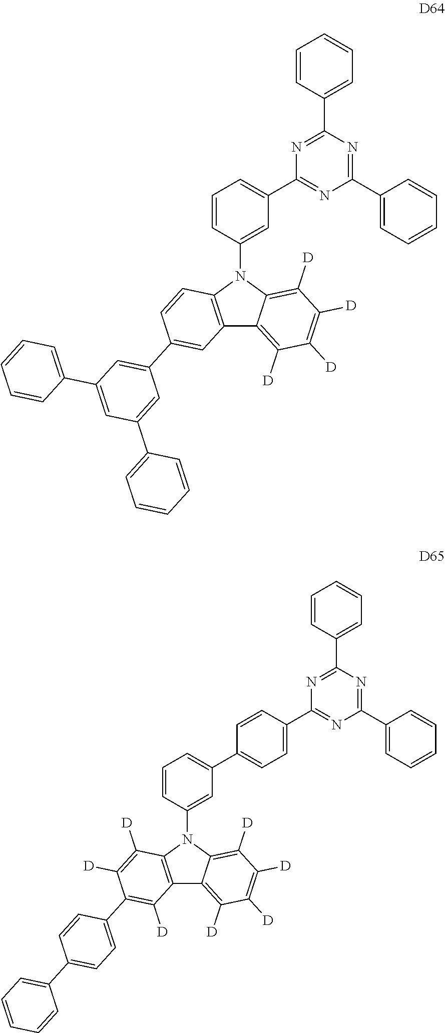 [SMILES: [2H]c1c([2H])c([2H])c2c(c1[2H])c1cc(-c3cc(-c4ccccc4)cc(-c4ccccc4)c3)ccc1n2-c1cccc(-c2nc(-c3ccccc3)nc(-c3ccccc3)n2)c1 ; [2H]c1c([2H])c([2H])c2c(c1[2H])c1c([2H])c(-c3ccc(-c4ccccc4)cc3)c([2H])c([2H])c1n2-c1cccc(-c2ccc(-c3nc(-c4ccccc4)nc(-c4ccccc4)n3)cc2)c1]

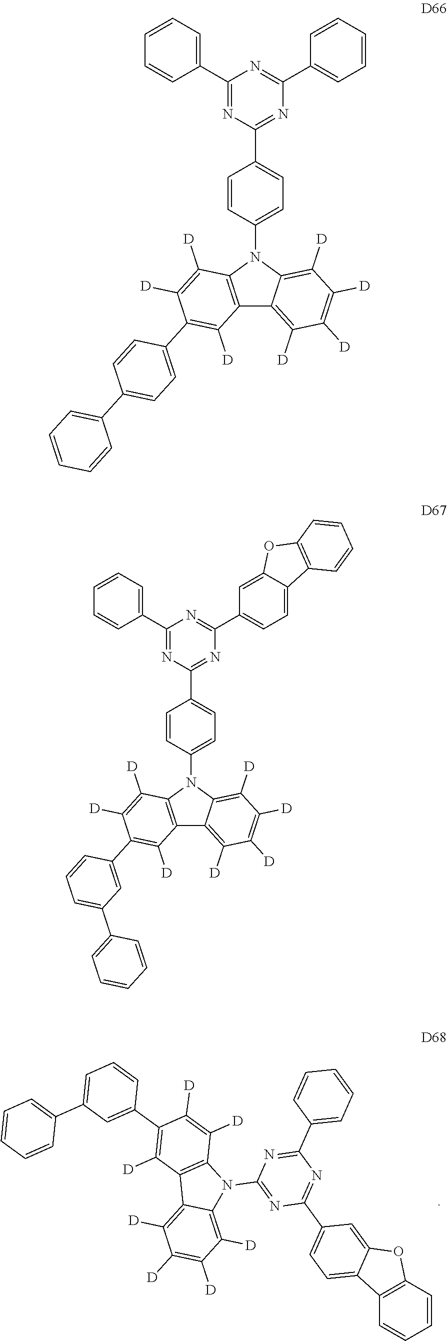 [SMILES: [2H]c1c([2H])c([2H])c2c(c1[2H])c1c([2H])c(-c3ccc(-c4ccccc4)cc3)c([2H])c([2H])c1n2-c1ccc(-c2nc(-c3ccccc3)nc(-c3ccccc3)n2)cc1 ; [2H]c1c([2H])c([2H])c2c(c1[2H])c1c([2H])c(-c3cccc(-c4ccccc4)c3)c([2H])c([2H])c1n2-c1ccc(-c2nc(-c3ccccc3)nc(-c3ccc4c(c3)oc3ccccc34)n2)cc1 ; [2H]c1c([2H])c([2H])c2c(c1[2H])c1c([2H])c(-c3cccc(-c4ccccc4)c3)c([2H])c([2H])c1n2-c1nc(-c2ccccc2)nc(-c2ccc3c(c2)oc2ccccc23)n1]

The present disclosure further provides a composition, and the composition contains a first compound and a second compound.

The first compound has a structure shown in the Formula 1, and the second compound has a structure shown in a Formula 2:

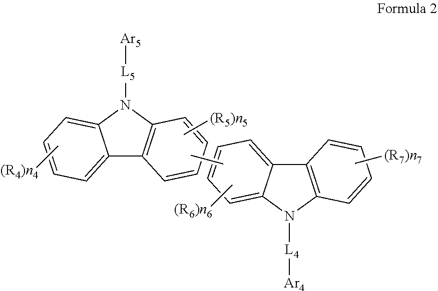

where each R₄, each R₅, each R₆ and each R₇ are respectively and independently selected from a hydrogen, a deuterium, a halogen group, a cyano, an aryl with 6 to 20 carbon atoms, a deuteroaryl with 6 to 20 carbon atoms, an alkyl with 1 to 10 carbon atoms, a deuteroalkyl with 1 to 10 carbon atoms, a haloalkyl with 1 to 10 carbon atoms, and a cycloalkyl with 3 to 10 carbon atoms;
n₄ represents the number of substituents R₄, n₄ is selected from 1, 2, 3 or 4, and in the case where n₄ is greater than 1, any two R₄ are the same or different;
n₅ represents the number of substituents R₅, n₅ is selected from 1, 2 or 3, and in the case where n₅ is greater than 1, any two R₅ are the same or different;
n₆ represents the number of substituents R₆, n₆ is selected from 1, 2 or 3, and in the case where n₆ is greater than 1, any two R₆ are the same or different;
n₇ represents the number of substituents R₇, n₇ is selected from 1, 2, 3 or 4, and in the case where n₇ is greater than 1, any two R₇ are the same or different;
L₄ and L₅ are the same or different, and are respectively and independently selected from a single bond, a substituted or unsubstituted arylene with 6 to 30 carbon atoms, and a substituted or unsubstituted heteroarylene with 3 to 30 carbon atoms;
Ar₄ and Ar₅ are the same or different, and are respectively and independently selected from a substituted or unsubstituted aryl with 6 to 30 carbon atoms, and a substituted or unsubstituted heteroaryl with 3 to 30 carbon atoms; and
substituent(s) in L₄, L₅, Ar₄ and Ar₅ are the same or different, and are respectively and independently selected from a deuterium, a halogen group, a cyano, a heteroaryl with 3 to 20 carbon atoms, an aryl with 6 to 20 carbon atoms, a deuteroaryl with 6 to 20 carbon atoms, a trialkylsilyl with 3 to 12 carbon atoms, an alkyl with 1 to 10 carbon atoms, a deuteroalkyl with 1 to 10 carbon atoms, a haloalkyl with 1 to 10 carbon atoms, a cycloalkyl with 3 to 10 carbon atoms, a heterocycloalkyl with 2 to 10 carbon atoms, and an alkoxy with 1 to 10 carbon atoms.

In some implementations of the present disclosure, the second compound has a structure shown in a Formula 2-3-3:

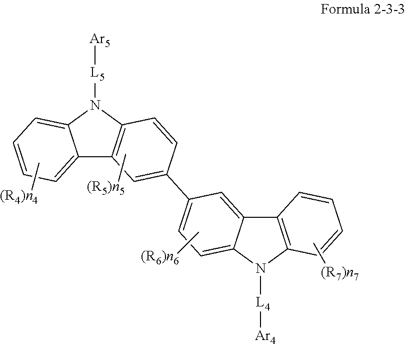

In some implementations of the present disclosure, in the second compound, each R₄, each R₅, each R₆, and each R₇ are respectively and independently selected from a hydrogen, a deuterium, a fluorine, a methyl, an ethyl, a n-propyl, an isopropyl, a tert-butyl, a phenyl, a naphthyl, a biphenyl and a pentadeuterophenyl.

In some implementations of the present disclosure, in the second compound, each R₄, each R₅, each R₆, and each R₇ are respectively and independently selected from a hydrogen, a deuterium, a fluorine, a cyano, a methyl, an ethyl, a n-propyl, an isopropyl, a tert-butyl, and a group consisting of the following groups:

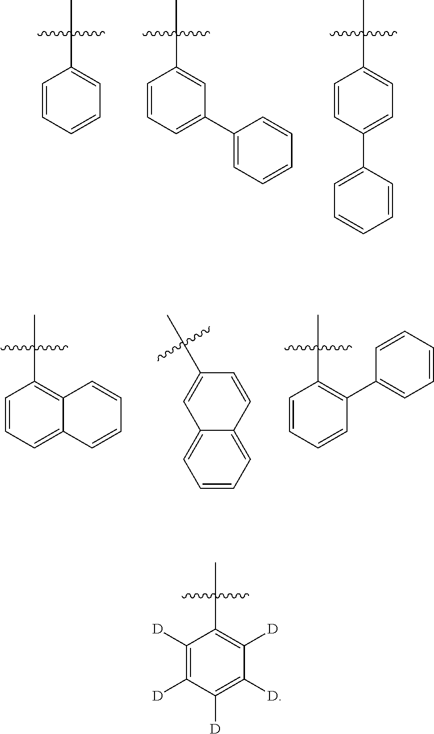

In some implementations of the present disclosure, in the second compound, L₄ and L₅ are respectively and independently selected from a single bond, a substituted or unsubstituted arylene with 6 to 20 carbon atoms, and a substituted or unsubstituted heteroarylene with 12 to 20 carbon atoms.

Optionally, substituent(s) in L₄ and L₅ are respectively and independently independently selected from a deuterium, a halogen group, a cyano, a alkyl with 1 to 5 carbon atoms and a phenyl.

In some other implementations of the present disclosure, in the second compound, L₄ and L₅ are respectively and independently selected from a single bond, a substituted or unsubstituted phenylene, a substituted or unsubstituted naphthylene, a substituted or unsubstituted biphenylene, a substituted or unsubstituted dibenzofurylene, a substituted or unsubstituted dibenzothienylene, and a substituted or unsubstituted carbazolylene.

Optionally, substituent(s) in L₄ and L₅ are respectively and independently selected from a deuterium, a fluorine, a cyano, a methyl, an ethyl, a n-propyl, an isopropyl, a tert-butyl, a phenyl and a pentadeuterophenyl.

In some implementations of the present disclosure, in the second compound, L₄ and L₅ are respectively and independently selected from a single bond, and a substituted or unsubstituted group U, where the unsubstituted group U is selected from a group consisting of the following groups:

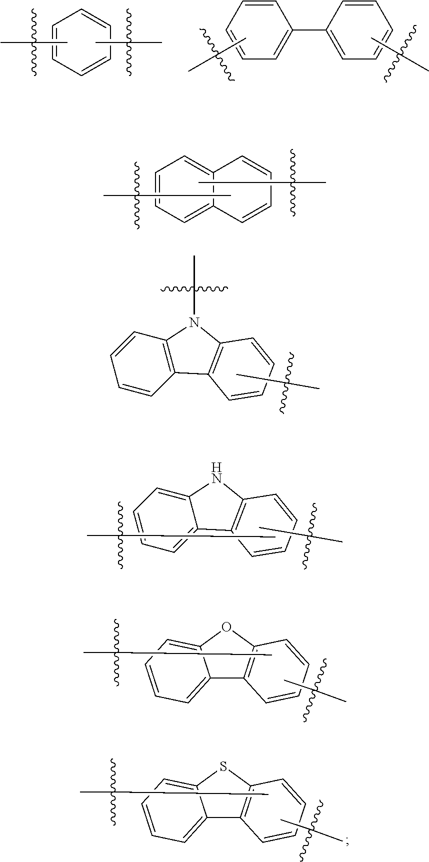

where

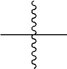

represents a chemical bond; the substituted group U has one or more substituents, and the substituents are each independently selected from a deuterium, a cyano, fluorine, a methyl, an ethyl, a n-propyl, an isopropyl, a tert-butyl and a phenyl; and when the number of the substituents on U is greater than 1, the substituents are the same or different.

Optionally, L₄ and L₅ are respectively and independently selected from a single bond or a group consisting of the following groups:

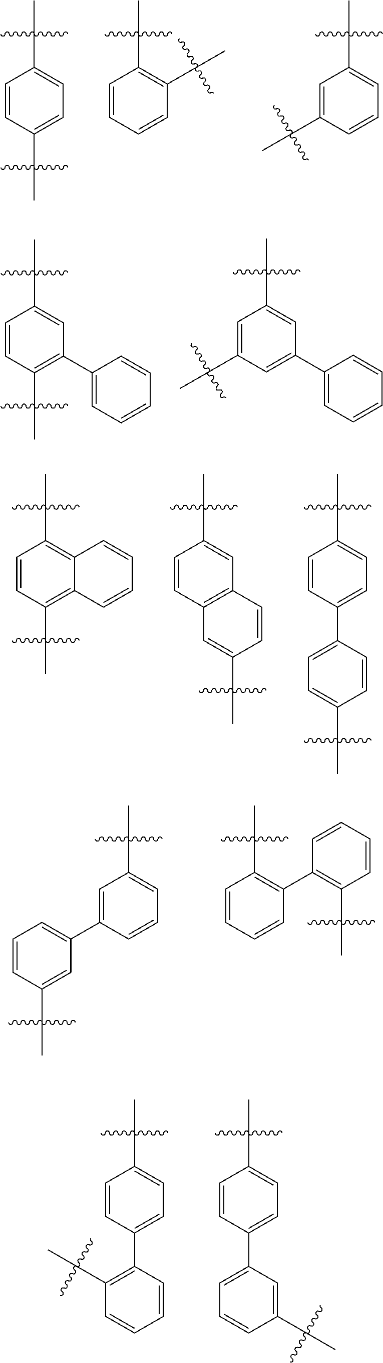

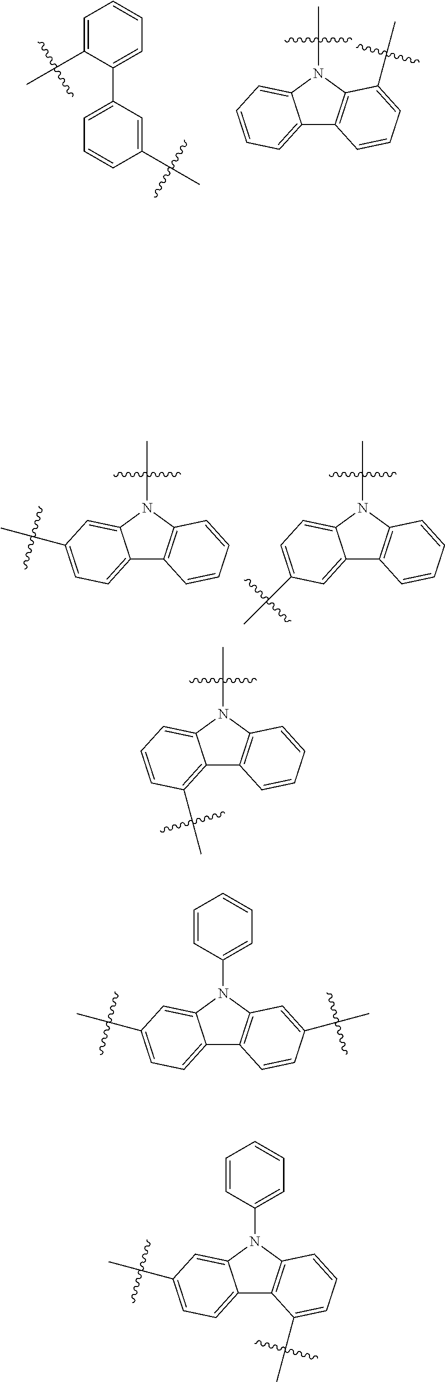

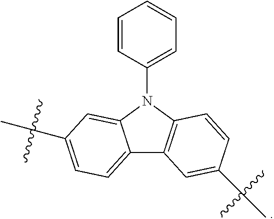 [SMILES: *c1ccc2c(c1)c1ccc(*)cc1n2-c1ccccc1 ; "."]

In some implementations of the present disclosure, in the second compound, Ar₄ and Ar₅ are respectively and independently selected from a substituted or unsubstituted aryl with 6 to 20 carbon atoms, and a substituted or unsubstituted heteroaryl with 12 to 20 carbon atoms.

Optionally, substituent(s) in Ar₄ and Ar₅ are respectively and independently selected from a deuterium, a halogen group, an alkyl with 1 to 5 carbon atoms, a phenyl and a pentadeuterophenyl.

In some other implementations of the present disclosure, in the second compound, Ar₄ and Ar₅ are respectively and independently selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted dibenzofuranyl, a substituted or unsubstituted dibenzothienyl, a substituted or unsubstituted carbazolyl and a substituted or unsubstituted triphenylene.

Optionally, substituent(s) in Ar₄ and Ar₅ are respectively and independently selected from a deuterium, a fluorine, a cyano, a halogen group, a methyl, an ethyl, a n-propyl, an isopropyl, a tert-butyl, a phenyl and a pentadeuterophenyl.

In some implementations of the present disclosure, in the second compound, Ar₄ and Ar₅ are respectively and independently selected from a substituted or unsubstituted group G, and the unsubstituted group G is selected from a group consisting of the following groups:

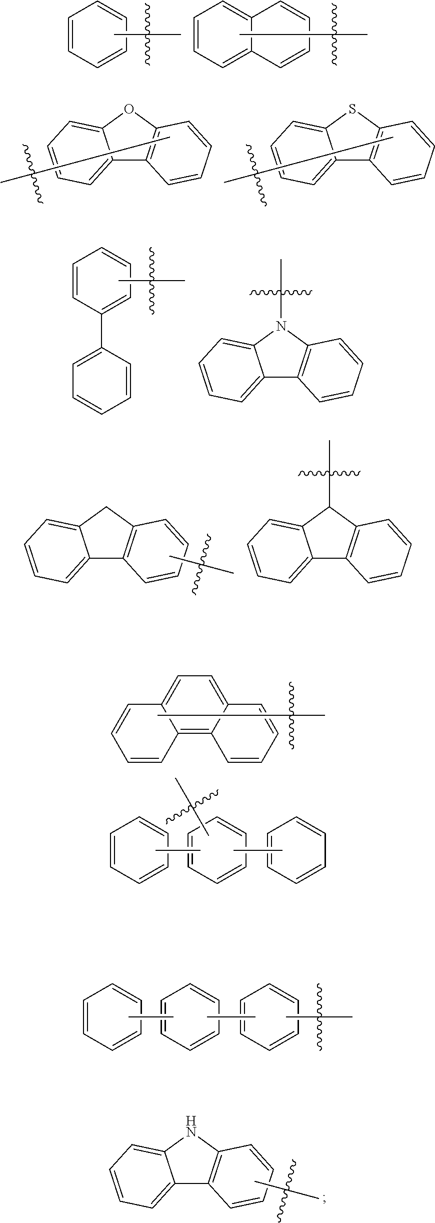

where

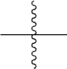

represents a chemical bond; the substituted group G has one or more substituents, and the substituents are each independently selected from a deuterium, a cyano, a fluorine, a methyl, an ethyl, a n-propyl, an isopropyl, a tert-butyl, a phenyl and a pentadeuterophenyl; and when the number of the substituents on G is greater than 1, the substituents are the same or different.

Optionally, in the second compound, Ar₄ and Ar₅ are respectively and independently selected from a group consisting of the following groups:

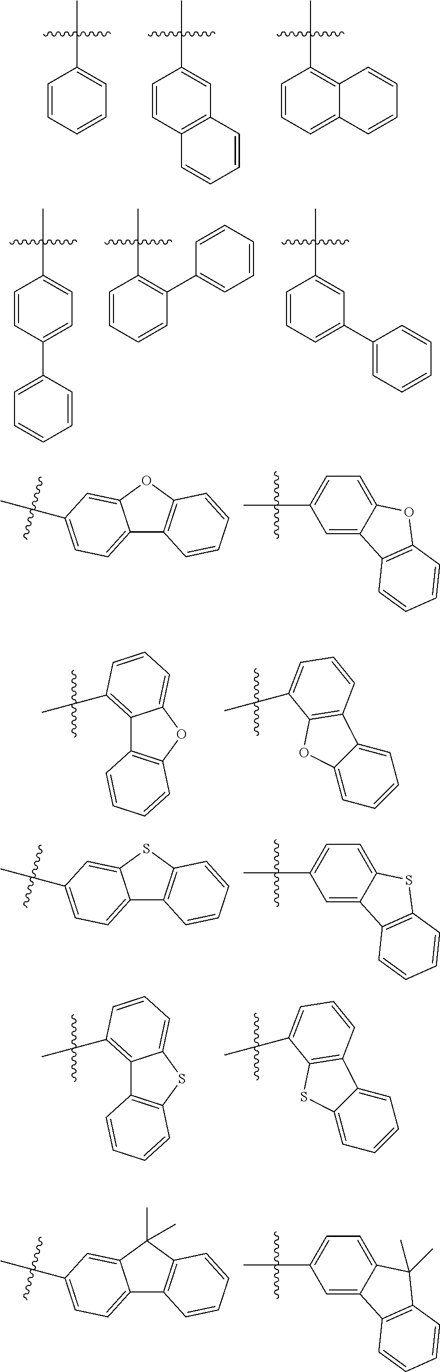

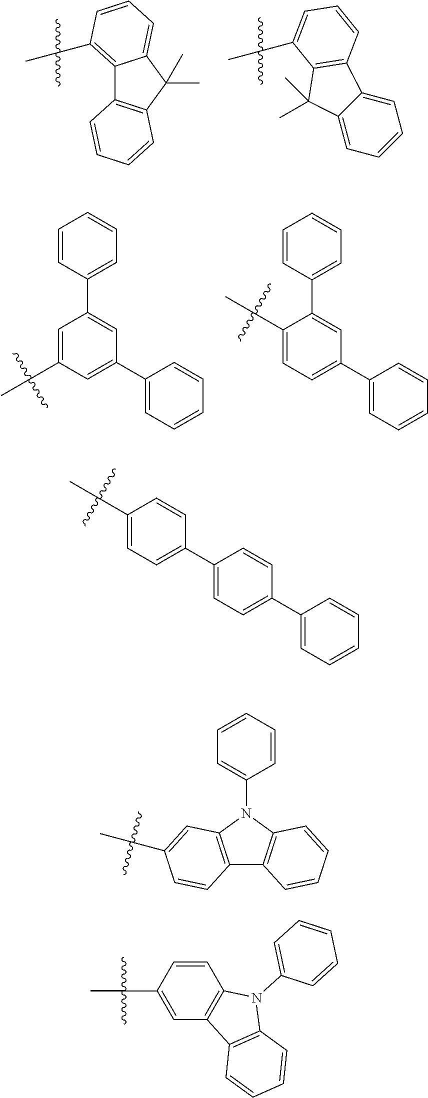

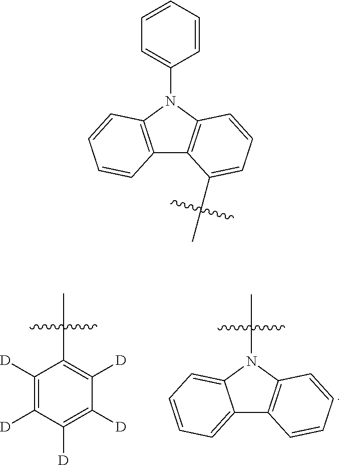 [SMILES: *c1cccc2c1c1ccccc1n2-c1ccccc1 ; *c1c([2H])c([2H])c([2H])c([2H])c1[2H] ; *n1c2ccccc2c2ccccc21]

In some implementations of the present disclosure,

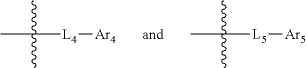

are respectively and independently selected from a group consisting of the following groups:

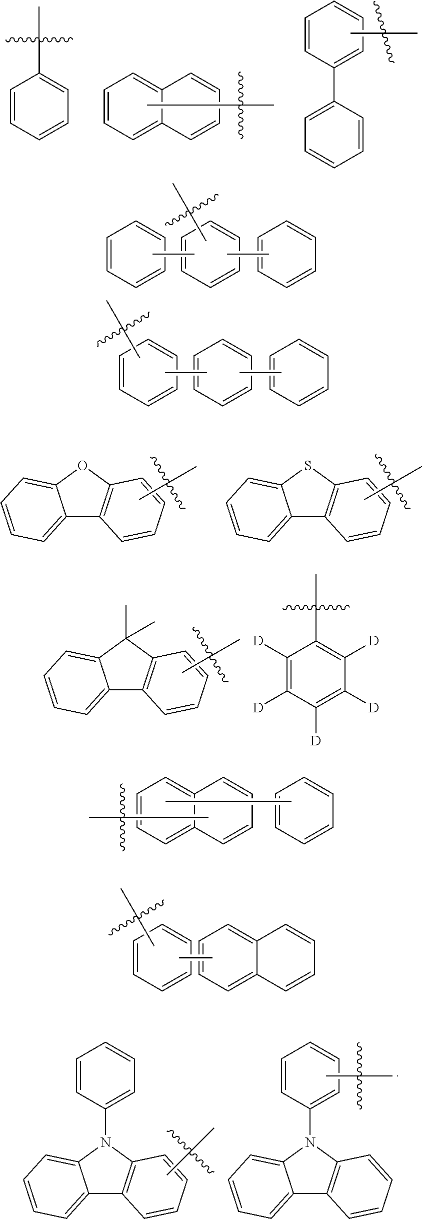

Specifically,

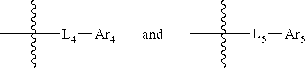

are respectively and independently selected from a group consisting of the following groups:

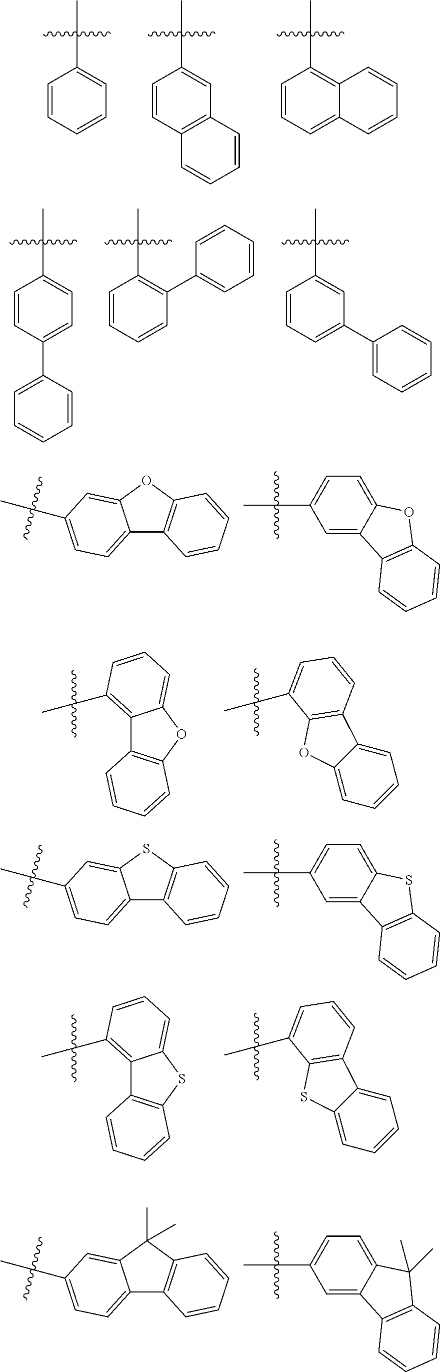

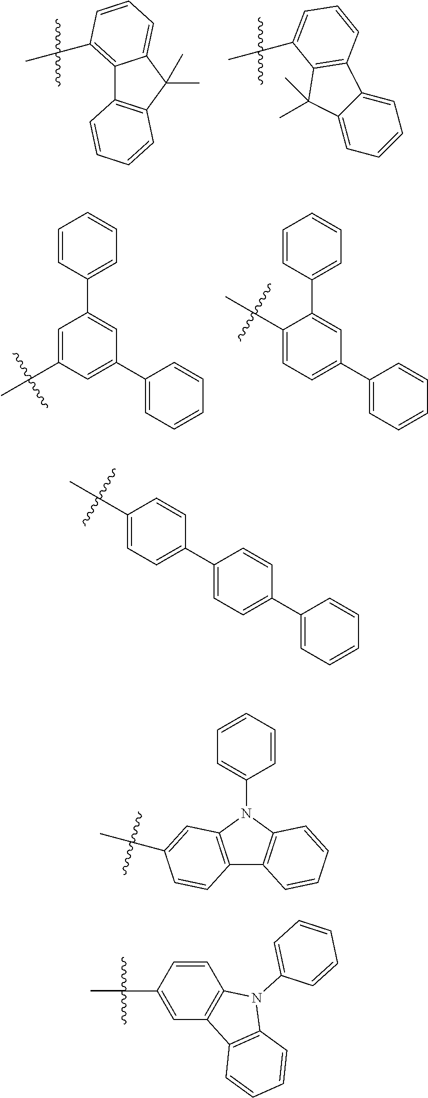

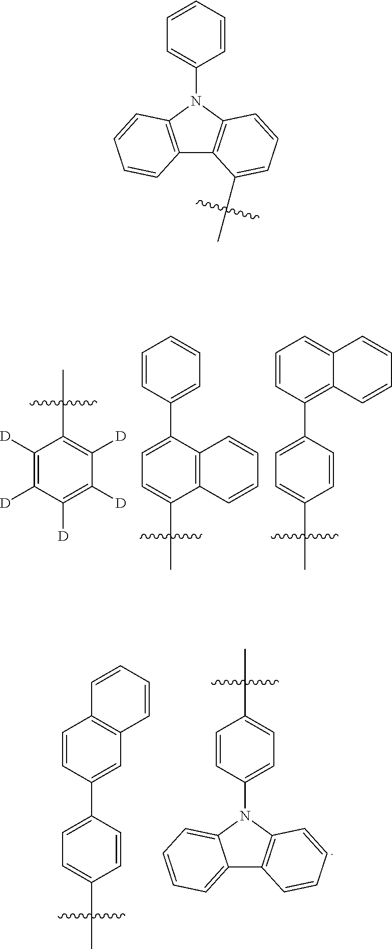

In some implementations of the present disclosure, the second compound is selected from a group consisting of the following compounds:

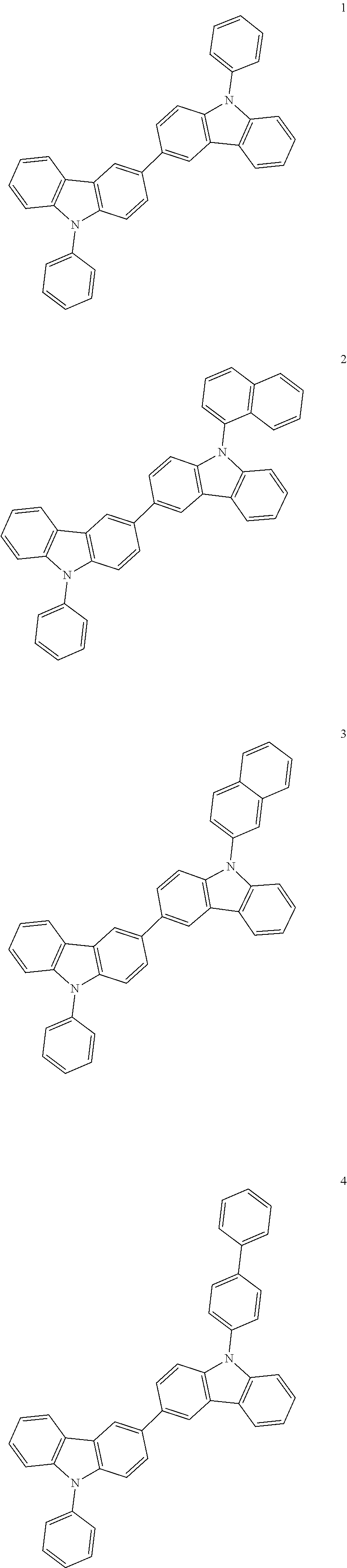 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; c1ccc(-c2ccc(-n3c4ccccc4c4cc(-c5ccc6c(c5)c5ccccc5n6-c5ccccc5)ccc43)cc2)cc1]

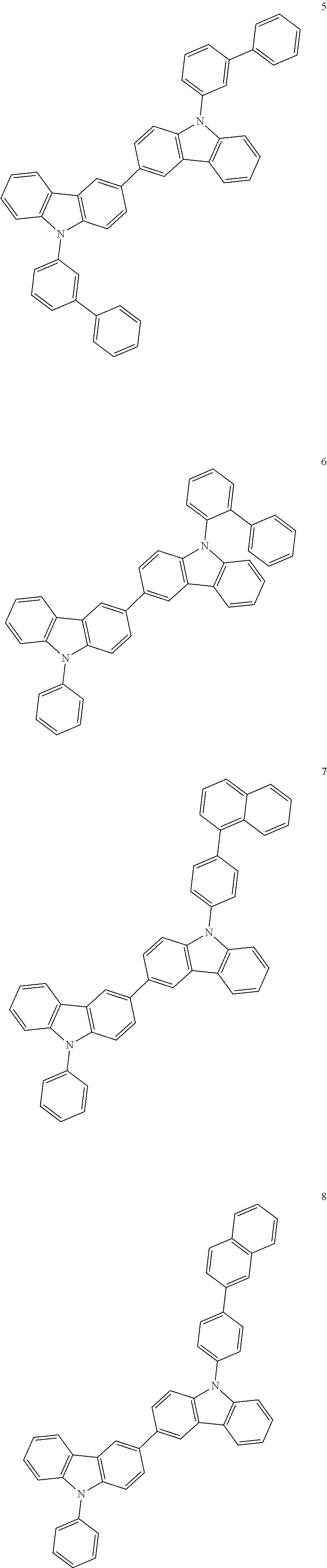 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

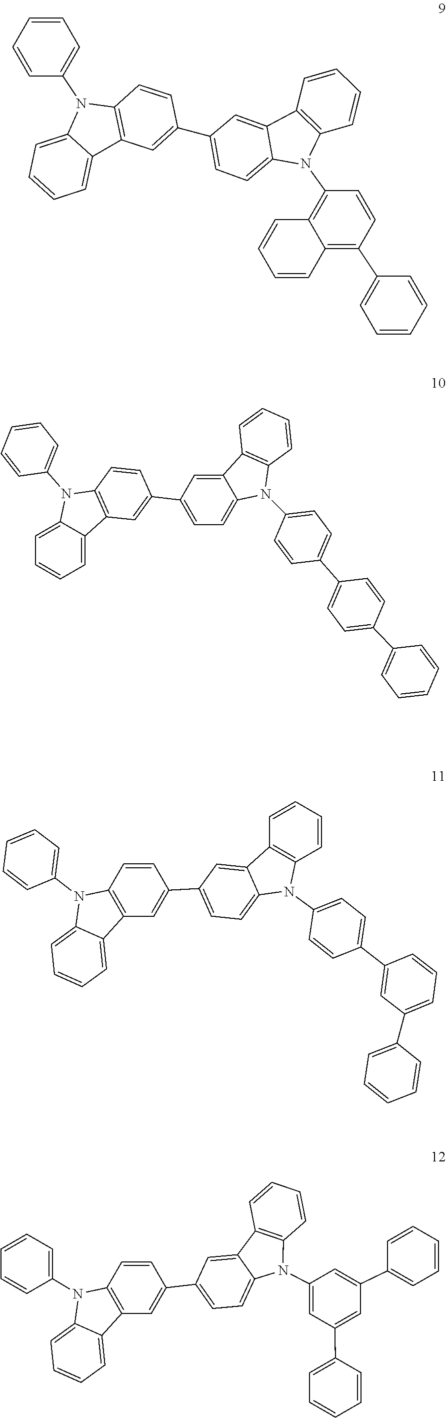 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

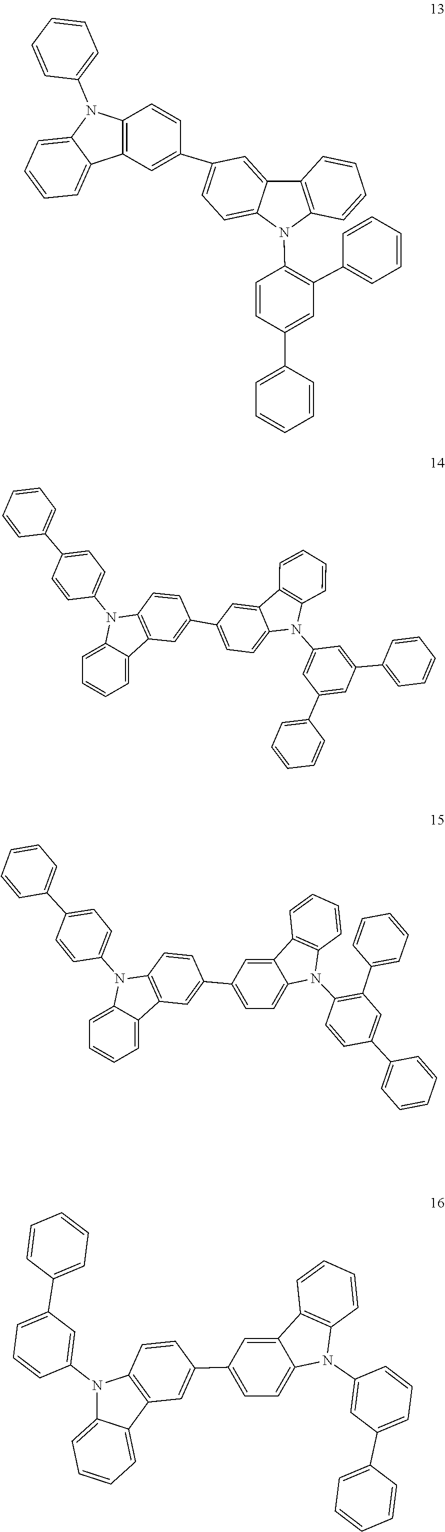 [SMILES: c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

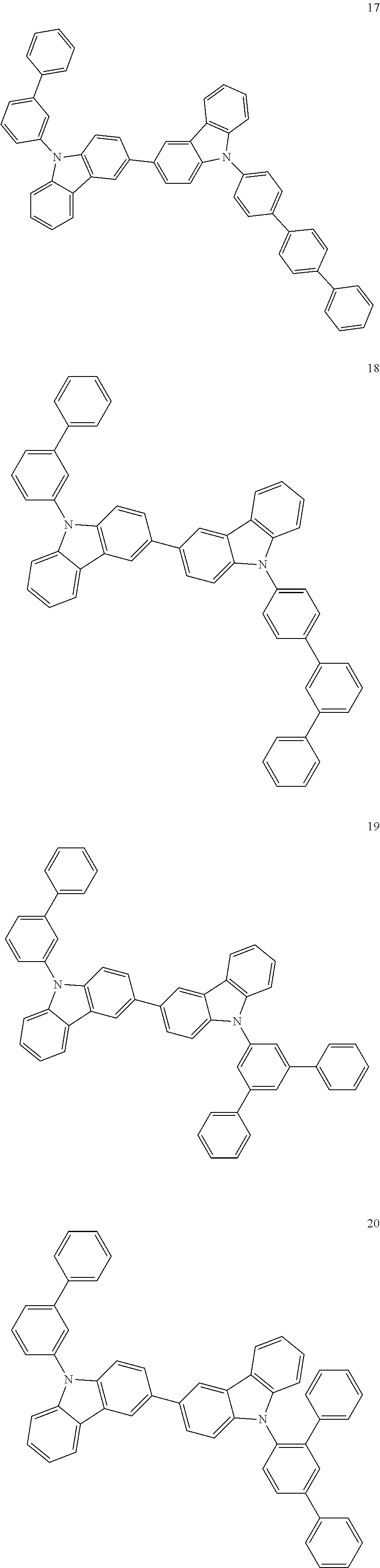 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc(-c6ccccc6)cc5-c5ccccc5)ccc43)c2)cc1]

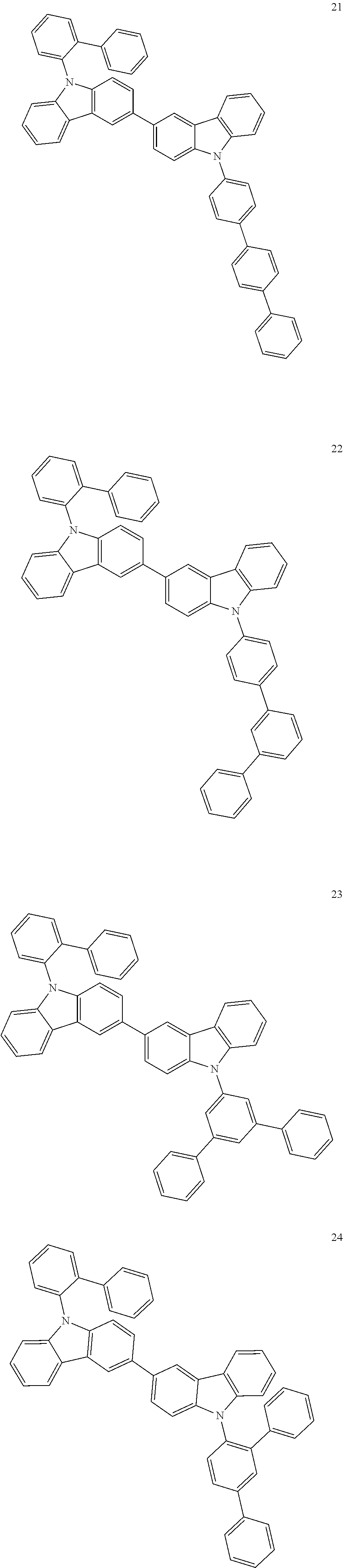 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cccc(-c3ccc(-n4c5ccccc5c5cc(-c6ccc7c(c6)c6ccccc6n7-c6ccccc6-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)c(-c3ccccc3)c2)cc1]

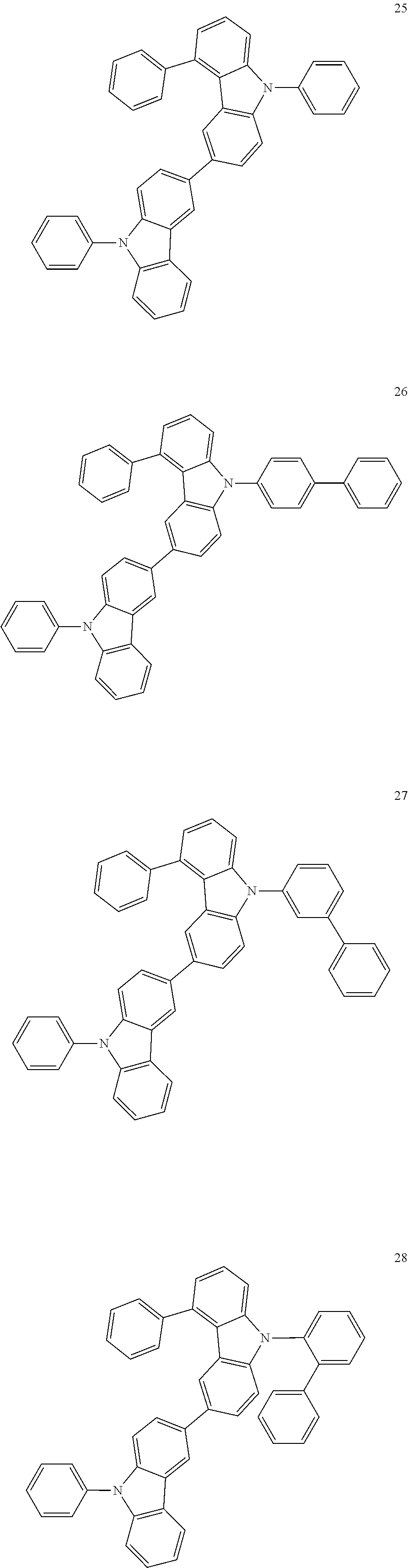 [SMILES: c1ccc(-c2cccc3c2c2cc(-c4ccc5c(c4)c4ccccc4n5-c4ccccc4)ccc2n3-c2ccccc2)cc1 ; c1ccc(-c2ccc(-n3c4ccc(-c5ccc6c(c5)c5ccccc5n6-c5ccccc5)cc4c4c(-c5ccccc5)cccc43)cc2)cc1 ; c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2ccccc2-n2c3ccc(-c4ccc5c(c4)c4ccccc4n5-c4ccccc4)cc3c3c(-c4ccccc4)cccc32)cc1]

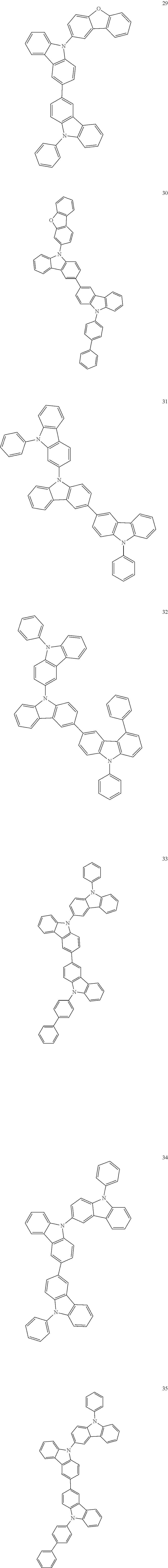

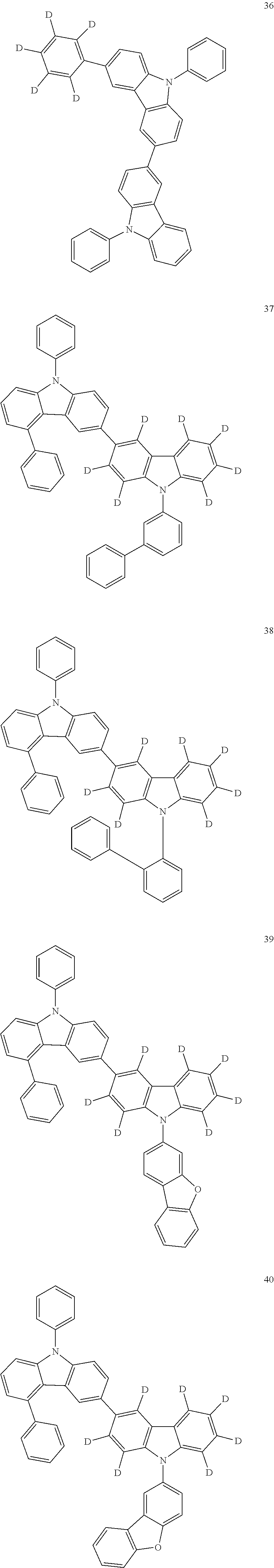

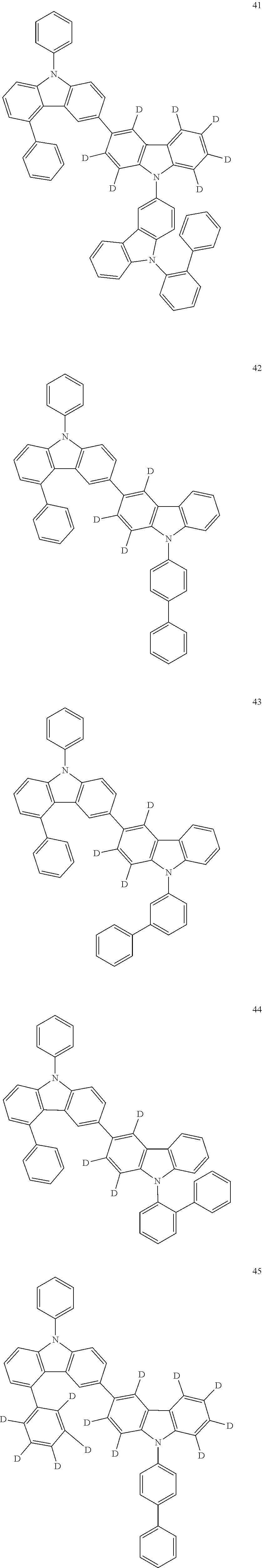

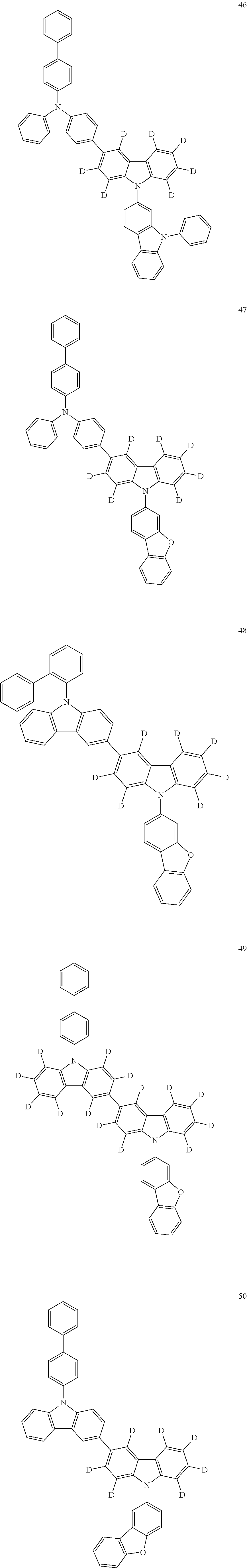

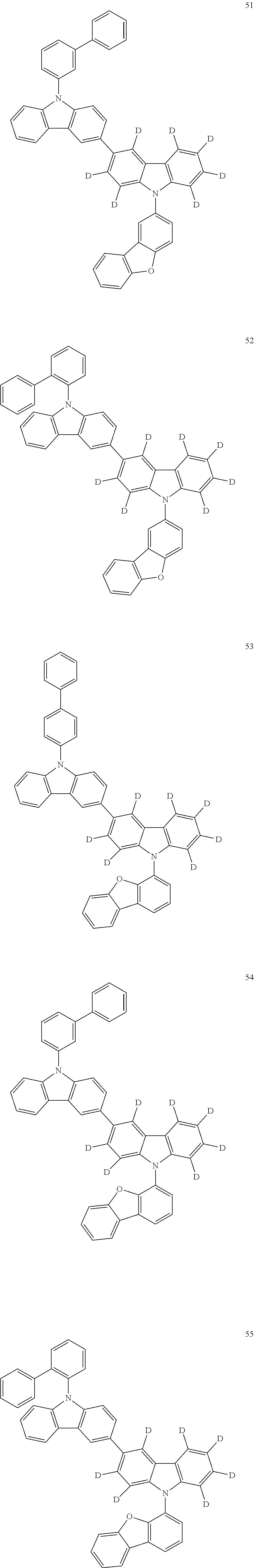

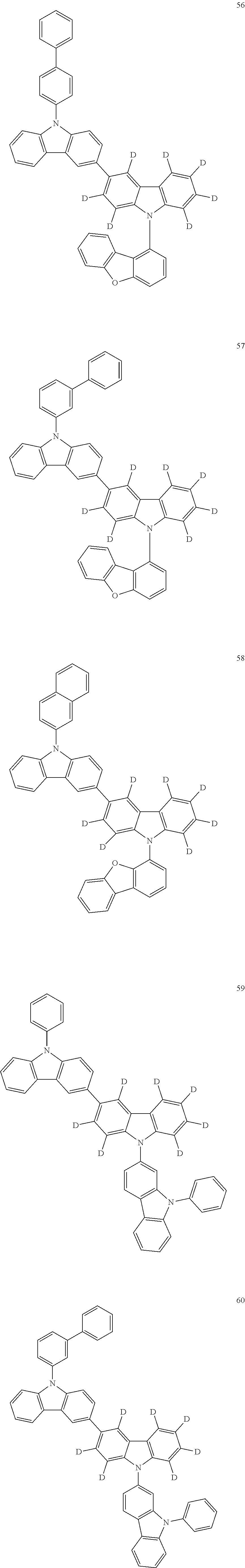

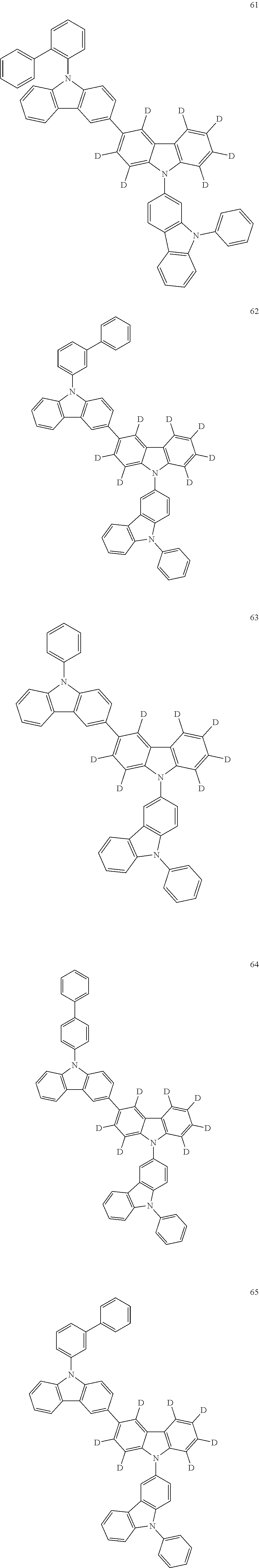

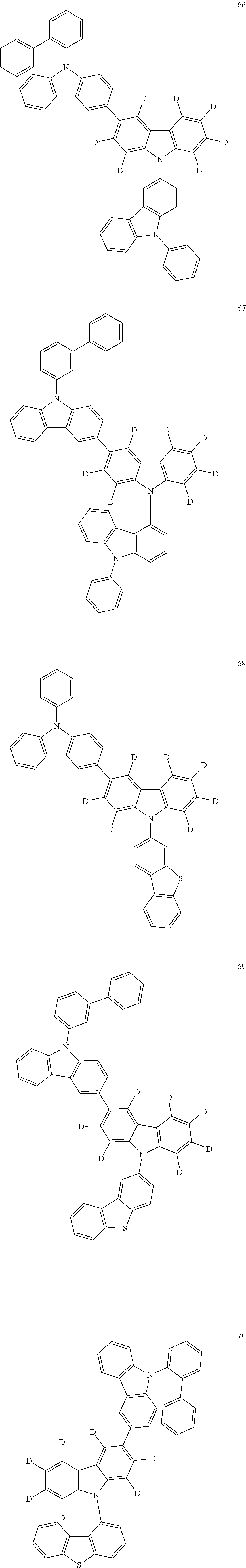

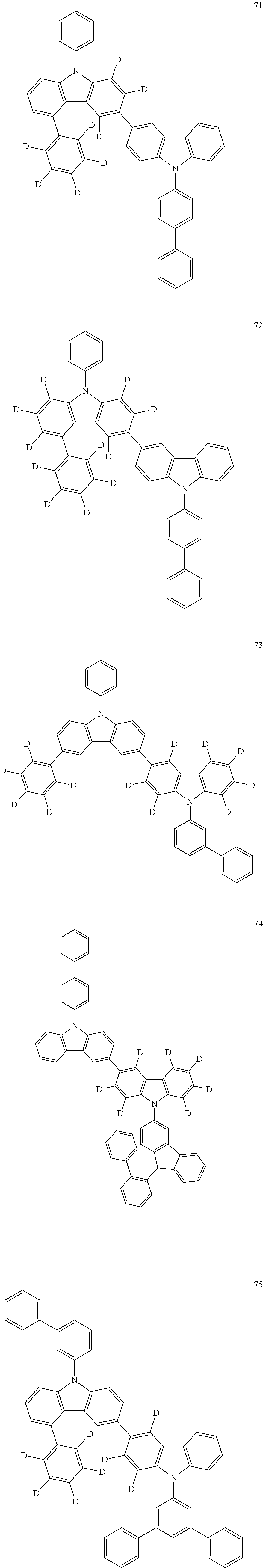

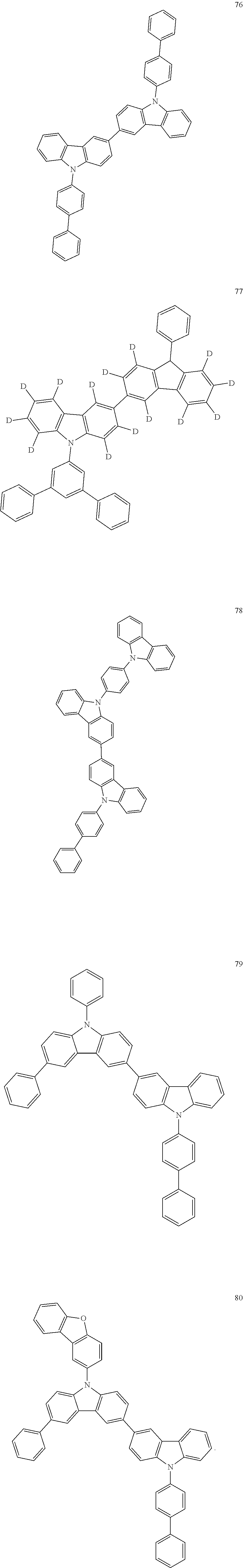

Optionally, the composition is a mixture of the first compound and the second compound. For example, the first compound and the second compound may be mixed evenly through mechanical stirring to form the mixture.

The relative contents of the two types of compounds in the composition are not particularly limited in the present disclosure, and may be selected according to specific applications of an organic electroluminescent device. Typically, with a total weight of the composition as the reference, the mass percentage content of the first compound may be 1% to 99%, and the mass percentage content of the second compound may be 1% to 99%. For example, in the composition, a mass ratio of the first compound to the second compound may be 1:99, 20:80, 30:70, 40:60, 45:55, 50:50, 55:45, 60:40, 70:30, 80:20, 99:1, etc.

In some implementations of the present disclosure, the composition is composed of the first compound and the second compound, where with a total weight of the composition as the reference, the mass percentage content of the first compound may be 20% to 80%, and the mass percentage content of the second compound may be 20% to 80%.

In some preferred implementations, in the composition, with a total weight of the composition as the reference, the mass percentage content of the first compound is 30% to 60%, and the mass percentage content of the second compound is 40% to 70%. In this case, applying the composition in an organic electroluminescent device may enable the device to have both high luminous efficiency and a long service life. Preferably, with a total weight of the composition as the reference, the mass percentage content of the first compound is 40% to 60%, and the mass percentage content of the second compound is 40% to 60%. More preferably, the mass percentage content of the first compound is 40% to 50%, and the mass percentage content of the second compound is 50% to 60%.

The present disclosure further provides an application of the composition as a host material of an organic light-emitting layer of an organic electroluminescent device.

The present disclosure further provides an organic electroluminescent device, including an anode and a cathode arranged oppositely, as well as at least one functional layer located between the anode and the cathode. The functional layer contains the organic compound shown in Formula 1 of the present disclosure or a composition containing a first compound and a second compound (the compound shown in Formula 2).

In an implementation of the present disclosure, the functional layer contains an organic light-emitting layer, and the organic light-emitting layer contains the organic compound shown in Formula 1 of the present disclosure.

In an implementation of the present disclosure, the functional layer contains an organic light-emitting layer, and the organic light-emitting layer contains the composition containing the first compound and the second compound provided by the present disclosure.

In an implementation of the present disclosure, the organic electroluminescent device is a phosphorescent device.

In a specific implementation of the present disclosure, the organic electroluminescent device is a green organic electroluminescent device.

In some implementations of the present disclosure, the organic electroluminescent device sequentially includes an anode (ITO substrate), a hole transport layer, a hole auxiliary layer, an organic light-emitting layer, an electron transport layer, an electron injection layer, a cathode (a Mg—Ag mixture) and an organic capping layer.

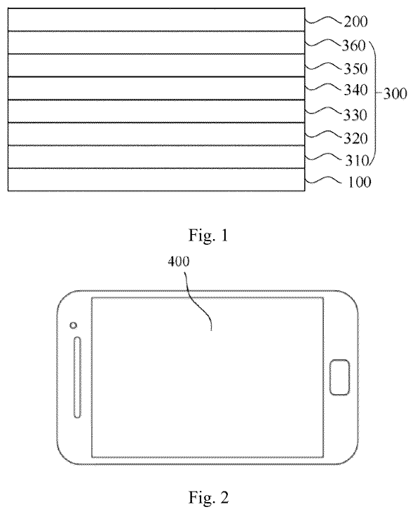

In a specific implementation of the present disclosure, as shown in FIG. 1, the organic electroluminescent device of the present disclosure includes an anode 100, a cathode 200 and at least one functional layer 300 located between an anode layer and a cathode layer, and the functional layer 300 includes a hole injection layer 310, a hole transport layer 320, a hole auxiliary layer 330, an organic light-emitting layer 340, an electron transport layer 350 and an electron injection layer 360.

Optionally, the anode 100 includes the following anode materials, preferably a material with a large work function that facilitates hole injection into the functional layer. Specific instances of the anode material include: metal, such as nickel, platinum, vanadium, chromium, copper, zinc and gold or their alloys; metal oxides, such as zinc oxide, indium oxide, indium tin oxide (ITO) and indium zinc oxide (IZO); combined metals and oxides, such as ZnO:Al or SnO₂:Sb; or conductive polymers, such as poly(3-methylthiophene), poly[3,4-(ethylene-1,2-dioxy)thiophene](PEDT), polypyrrole and polyaniline, but are not limited to these. Preferably, the anode material includes a transparent electrode containing indium tin oxide (ITO) as an anode.

Optionally, the hole transport layer 320 may include one or more hole transport materials, which may be selected from carbazole polymers, carbazole linked triarylamine compounds, or other types of compounds, which is not specially limited in the present disclosure. For example, in some implementations of the present disclosure, the hole transport layer 320 is composed of HT-23.

Optionally, the hole auxiliary layer 330 may include one or more hole transport materials, which may be selected from carbazole polymers, carbazole-linked triarylamine compounds, or other types of compounds, which is not specially limited in the present disclosure. For example, in some implementations of the present disclosure, the hole auxiliary layer 330 is composed of HT-24. The hole auxiliary layer is also referred to as a second hole transport layer, a hole buffer layer, a hole adjusting layer or an electron block layer.

Optionally, the organic light-emitting layer 340 may be composed of a singular light-emitting material, and may also include a host material and a guest material. Optionally, the organic light-emitting layer 340 is composed of a host material and a guest material, holes and electrons injected into the organic light-emitting layer 340 may be compounded in the organic light-emitting layer 340 to form excitons, the excitons transfer energy to the host material, the host material transfers the energy to the guest material, and then the guest material is made to emit light.

The guest material of the organic light-emitting layer 340 may be a compound with a condensed aryl ring or its derivatives, a compound with a heteroaryl ring or its derivatives, an aromatic amine derivative or other materials, which is not specially limited in the present disclosure.

In some implementations of the present disclosure, in a green organic electroluminescent device, the organic light-emitting layer 340 contains the organic compound of the present disclosure, a second compound and a guest material GD.

The electron transport layer 350 may be of a single-layer structure or a multi-layer structure, and may include one or more electron transport materials which may be selected from a benzoimidazole derivative, an oxadiazole derivative, a quinoxaline derivative or other electron transport materials, which is not specially limited in the present disclosure. For example, in some implementations of the present disclosure, the electron transport layer 350 may be composed of ET-01 and LiQ.

Optionally, the cathode 200 includes the following cathode materials, which are materials with a small work function that facilitates electron injection into the functional layer. Specific instances of the cathode materials include: metal, such as magnesium, calcium, sodium, potassium, titanium, indium, yttrium, lithium, gadolinium, aluminum, silver, tin and lead or their alloys; or multi-layer materials, such as LiF/Al, Liq/Al, LiO₂/Al, LiF/Ca, LiF/Al and BaF₂/Ca, but are not limited to these. Preferably, the cathode materials include a metal electrode containing silver and magnesium as a cathode.

Optionally, the hole injection layer 310 may further be arranged between the anode 100 and the hole transport layer 320 to enhance the capability of injecting holes into the hole transport layer 320. The hole injection layer 310 may select a benzidine derivative, a starburst-like arylamine compound, a phthalocyanine derivative or other materials, which is not specially limited in the present disclosure. In some implementations of the present disclosure, the hole injection layer 310 may be composed of PD and HT-23.

Optionally, the electron injection layer 360 may further be arranged between the cathode 200 and the electron transport layer 350 to enhance the capability of injecting electrons into the electron transport layer 350. The electron injection layer 360 may include inorganic materials such as alkali metal sulfide and alkali metal halide, or may include a complex of alkali metal and organics. In some implementations of the present disclosure, the electron injection layer 360 may include ytterbium (Yb).

The present disclosure further provides an electronic apparatus, including the organic electroluminescent device of the present disclosure.

For example, as shown in FIG. 2, the electronic apparatus provided by the present disclosure is a first electronic apparatus 400, and the first electronic apparatus 400 includes any organic electroluminescent device described in the above organic electroluminescent device implementation. The electronic apparatus may be a display apparatus, a lighting apparatus, an optical communication apparatus or other types of electronic apparatuses, for example, it may include but not limited to a computer screen, a mobile phone screen, a television, electronic paper, an emergency light, an optical module, etc. Since the first electronic apparatus 400 has the above organic electroluminescent device, they have the same beneficial effects, which are not repeated here in the present disclosure.

The present disclosure will be described in detail below in combination with examples, however, the following description is used to explain the present disclosure, instead of limiting the scope of the present disclosure in any manner.

### Synthesis of Intermediate ai

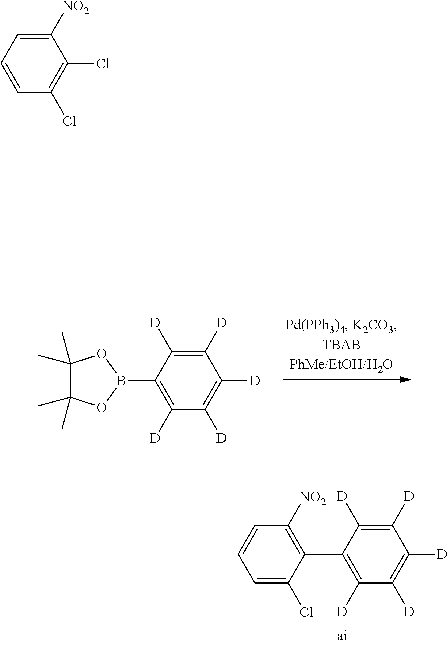 [SMILES: O=[N+]([O-])c1cccc(Cl)c1Cl ; [2H]c1c([2H])c([2H])c(B2OC(C)(C)C(C)(C)O2)c([2H])c1[2H] ; [2H]c1c([2H])c([2H])c(-c2c(Cl)cccc2[N+](=O)[O-])c([2H])c1[2H]]

Under the protection of nitrogen, 2,3-dichloronitrobenzene (20.0 g; 104.2 mmol), D5-pinacol phenylboronate (21.8 g; 104.2 mmol), tetrakis(triphenylphosphine)palladium (2.4 g; 2.1 mmol), potassium carbonate (28.8 g; 208.3 mmol), tetrabutylammonium bromide (6.7 g; 20.8 mmol), toluene (160 mL), ethanol (40 mL) and deionized water (40 mL) were added into a round-bottom flask, and a mixed liquor was heated to 75° C. to 80° C., and a reaction was carried out under stirring for 48 hours. The reaction solution was cooled to the room temperature, deionized water was added into the reaction solution, liquid separation was performed, an organic phase was washed and then dried with anhydrous magnesium sulfate, and a solvent was removed in a pressure reduction pressure; and an obtained crude product was purified by silica gel column chromatography purification using a dichloromethane/n-heptane mixed solvent as an eluent to obtain a Intermediate ai as a white solid (18.8 g; yield: 76%).

Referring to a synthesis method for the Intermediate ai, 2,3-dichloronitrobenzene was replaced with Reactant A to synthesize Intermediates shown in Table 1 below:

TABLE 1

| Intermediates No. | Reactant A | Structure | Yield (%) |
|---|---|---|---|
| bi | 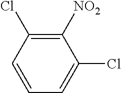 [SMILES: O=[N+]([O-])c1c(Cl)cccc1Cl] | 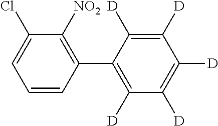 [SMILES: [2H]c1c([2H])c([2H])c(-c2cccc(Cl)c2[N+](=O)[O-])c([2H])c1[2H]] | 54 |
| ci | 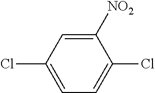 [SMILES: O=[N+]([O-])c1cc(Cl)ccc1Cl] | 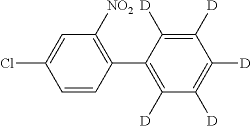 [SMILES: [2H]c1c([2H])c([2H])c(-c2ccc(Cl)cc2[N+](=O)[O-])c([2H])c1[2H]] | 69 |
| di | 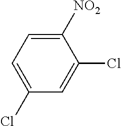 [SMILES: O=[N+]([O-])c1ccc(Cl)cc1Cl] | 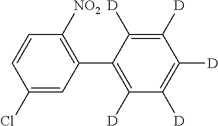 [SMILES: [2H]c1c([2H])c([2H])c(-c2cc(Cl)ccc2[N+](=O)[O-])c([2H])c1[2H]] | 74 |

### Synthesis of Intermediate aii

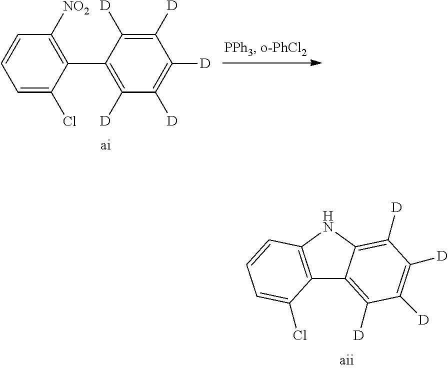 [SMILES: [2H]c1c([2H])c([2H])c(-c2c(Cl)cccc2[N+](=O)[O-])c([2H])c1[2H] ; [2H]c1c([2H])c([2H])c2c([nH]c3cccc(Cl)c32)c1[2H]]

Under the protection of nitrogen, the Intermediate ai (18.0 g; 75.4 mmol), triphenylphosphine (49.5 g; 188.5 mmol) and o-dichlorobenzene (150 mL) were added into a round-bottom flask, and under a stirring condition, a mixed liquor was heated to 175° C. to 180° C., and a reaction was carried out under stirring for 36 hours. The reaction solution was cooled to the room temperature, deionized water was added into the reaction solution, liquid separation was performed, an organic phase was washed and then dried with anhydrous magnesium sulfate, and a solvent was removed under a high-temperature pressure-reduced condition; and an obtained crude product was purified by silica gel column chromatography purification using a dichloromethane/n-heptane mixed solvent as an eluent to obtain a Intermediate aii as a white solid (11.1 g; yield: 72%).

Referring to a synthesis method for the Intermediate aii, the Intermediate ai was replaced with Reactant B to synthesize Intermediates shown in Table 2 below:

TABLE 2

| Intermediates No. | Reactant B | Structure | Yield (%) |
|---|---|---|---|
| bii | 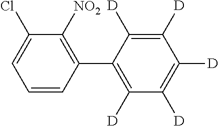 [SMILES: [2H]c1c([2H])c([2H])c(-c2cccc(Cl)c2[N+](=O)[O-])c([2H])c1[2H]] | 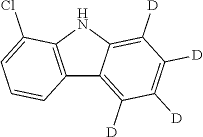 [SMILES: [2H]c1c([2H])c([2H])c2c([nH]c3c(Cl)cccc32)c1[2H]] | 65 |
| cii | 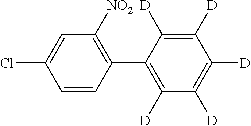 [SMILES: [2H]c1c([2H])c([2H])c(-c2ccc(Cl)cc2[N+](=O)[O-])c([2H])c1[2H]] | 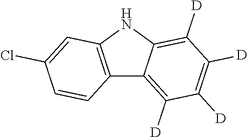 [SMILES: [2H]c1c([2H])c([2H])c2c([nH]c3cc(Cl)ccc32)c1[2H]] | 71 |
| dii | 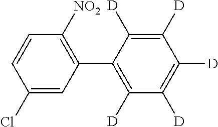 [SMILES: [2H]c1c([2H])c([2H])c(-c2cc(Cl)ccc2[N+](=O)[O-])c([2H])c1[2H]] | 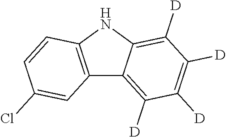 [SMILES: [2H]c1c([2H])c([2H])c2c([nH]c3ccc(Cl)cc32)c1[2H]] | 80 |

### Synthesis of Intermediate a1

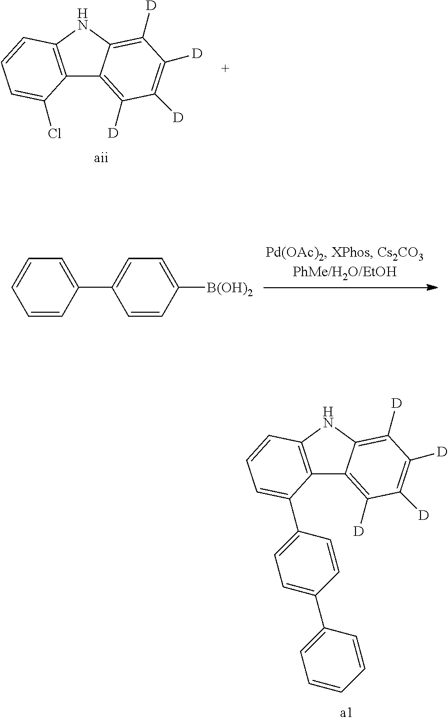 [SMILES: [2H]c1c([2H])c([2H])c2c([nH]c3cccc(Cl)c32)c1[2H] ; OB(O)c1ccc(-c2ccccc2)cc1 ; [2H]c1c([2H])c([2H])c2c([nH]c3cccc(-c4ccc(-c5ccccc5)cc4)c32)c1[2H]]

Intermediate aii (10.0 g; 48.6 mmol), 4-biphenylboronic acid (10.1 g; 51.1 mmol), palladium acetate (0.1 g; 0.5 mmol), 2-dicyclohexylphosphine-2′,4′,6′-triisopropyl biphenyl (0.5 g; 1.0 mmol), cesium carbonate (23.8 g; 72.9 mmol), toluene (80 mL), ethanol (20 mL) and deionized water (20 mL) were added into a round-bottom flask under nitrogen protection, and a mixed liquor was heated to 75° C. to 80° C., and a reaction was carried out under stirring for 48 hours. The reaction solution was cooled to the room temperature, deionized water was added, liquid separation was performed, an organic phase was washed and then dried with anhydrous magnesium sulfate, and a solvent was removed in a pressure reduction pressure; and an obtained crude product was purified by silica gel column chromatography purification using a dichloromethane/n-heptane mixed solvent as an eluent to obtain a Intermediate a1 as a white solid (12.1 g; yield: 77%).

Referring to a synthesis method for the Intermediate a1, the Intermediate aii was replaced with Reactant C and 4-biphenylboronic acid was replaced with Reactant D to synthesize Intermediates shown in Table 3 below:

TABLE 3

| Intermediates | | |
|---|---|---|
| No. | Reactant C | Reactant D |
| a2 | 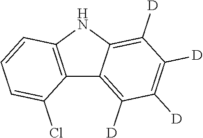 [SMILES: [2H]c1c([2H])c([2H])c2c([nH]c3cccc(Cl)c32)c1[2H]] | 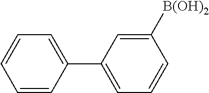 [SMILES: OB(O)c1cccc(-c2ccccc2)c1] |
| a3 | 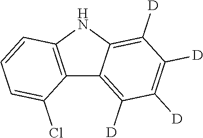 [SMILES: [2H]c1c([2H])c([2H])c2c([nH]c3cccc(Cl)c32)c1[2H]] | 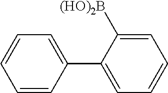 [SMILES: OB(O)c1ccccc1-c1ccccc1] |
| a4 | 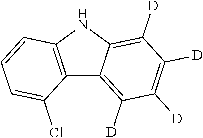 [SMILES: [2H]c1c([2H])c([2H])c2c([nH]c3cccc(Cl)c32)c1[2H]] | 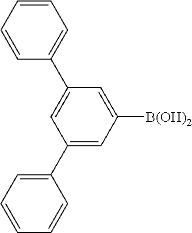 [SMILES: OB(O)c1cc(-c2ccccc2)cc(-c2ccccc2)c1] |
| b1 | 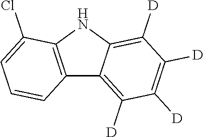 [SMILES: [2H]c1c([2H])c([2H])c2c([nH]c3c(Cl)cccc32)c1[2H]] | 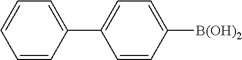 [SMILES: OB(O)c1ccc(-c2ccccc2)cc1] |
| b2 | 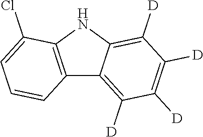 [SMILES: [2H]c1c([2H])c([2H])c2c([nH]c3c(Cl)cccc32)c1[2H]] | 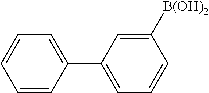 [SMILES: OB(O)c1cccc(-c2ccccc2)c1] |
| b3 | 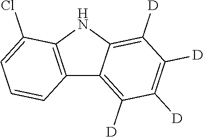 [SMILES: [2H]c1c([2H])c([2H])c2c([nH]c3c(Cl)cccc32)c1[2H]] | 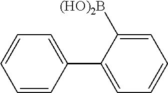 [SMILES: OB(O)c1ccccc1-c1ccccc1] |
| b4 | 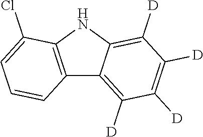 [SMILES: [2H]c1c([2H])c([2H])c2c([nH]c3c(Cl)cccc32)c1[2H]] | 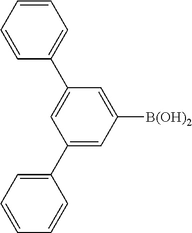 [SMILES: OB(O)c1cc(-c2ccccc2)cc(-c2ccccc2)c1] |
| b5 | 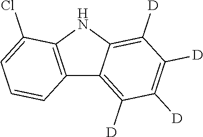 [SMILES: [2H]c1c([2H])c([2H])c2c([nH]c3c(Cl)cccc32)c1[2H]] | 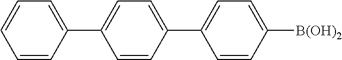 [SMILES: OB(O)c1ccc(-c2ccc(-c3ccccc3)cc2)cc1] |
| c1 | 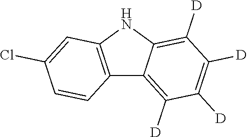 [SMILES: [2H]c1c([2H])c([2H])c2c([nH]c3cc(Cl)ccc32)c1[2H]] | 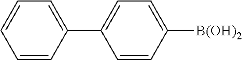 [SMILES: OB(O)c1ccc(-c2ccccc2)cc1] |
| c2 | 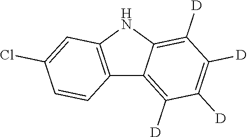 [SMILES: [2H]c1c([2H])c([2H])c2c([nH]c3cc(Cl)ccc32)c1[2H]] | 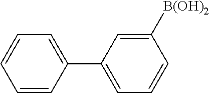 [SMILES: OB(O)c1cccc(-c2ccccc2)c1] |
| c3 | 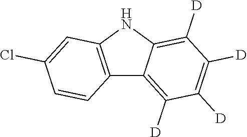 [SMILES: [2H]c1c([2H])c([2H])c2c([nH]c3cc(Cl)ccc32)c1[2H]] | 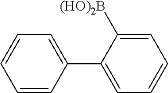 [SMILES: OB(O)c1ccccc1-c1ccccc1] |
| c4 | 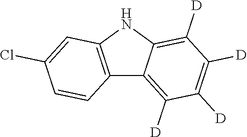 [SMILES: [2H]c1c([2H])c([2H])c2c([nH]c3cc(Cl)ccc32)c1[2H]] | 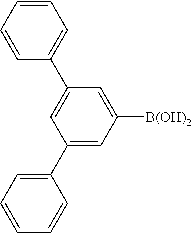 [SMILES: OB(O)c1cc(-c2ccccc2)cc(-c2ccccc2)c1] |
| d1 | 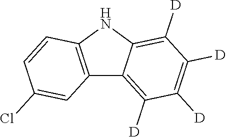 [SMILES: [2H]c1c([2H])c([2H])c2c([nH]c3ccc(Cl)cc32)c1[2H]] | 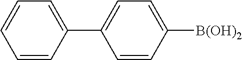 [SMILES: OB(O)c1ccc(-c2ccccc2)cc1] |
| d2 | 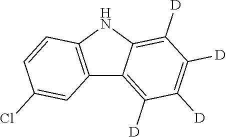 [SMILES: [2H]c1c([2H])c([2H])c2c([nH]c3ccc(Cl)cc32)c1[2H]] | 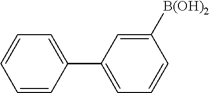 [SMILES: OB(O)c1cccc(-c2ccccc2)c1] |
| d3 | 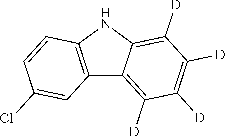 [SMILES: [2H]c1c([2H])c([2H])c2c([nH]c3ccc(Cl)cc32)c1[2H]] | 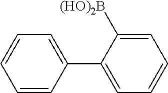 [SMILES: OB(O)c1ccccc1-c1ccccc1] |
| d4 | 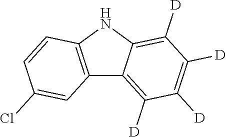 [SMILES: [2H]c1c([2H])c([2H])c2c([nH]c3ccc(Cl)cc32)c1[2H]] | 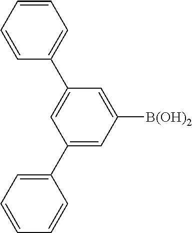 [SMILES: OB(O)c1cc(-c2ccccc2)cc(-c2ccccc2)c1] |

| Intermediates | | |
|---|---|---|
| No. | Structure | Yield (%) |
| a2 | 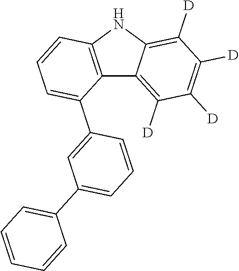 [SMILES: [2H]c1c([2H])c([2H])c2c([nH]c3cccc(-c4cccc(-c5ccccc5)c4)c32)c1[2H]] | 78 |
| a3 | 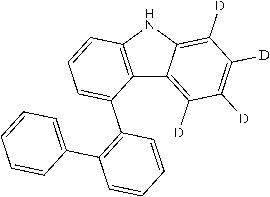 [SMILES: [2H]c1c([2H])c([2H])c2c([nH]c3cccc(-c4ccccc4-c4ccccc4)c32)c1[2H]] | 62 |
| a4 | 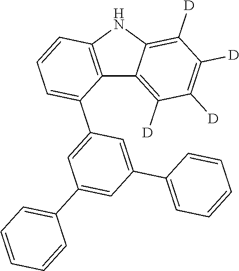 [SMILES: [2H]c1c([2H])c([2H])c2c([nH]c3cccc(-c4cc(-c5ccccc5)cc(-c5ccccc5)c4)c32)c1[2H]] | 65 |
| b1 | 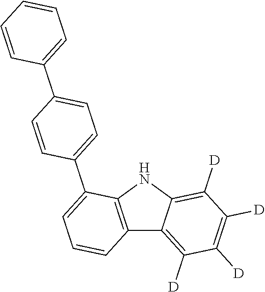 [SMILES: [2H]c1c([2H])c([2H])c2c([nH]c3c(-c4ccc(-c5ccccc5)cc4)cccc32)c1[2H]] | 70 |
| b2 | 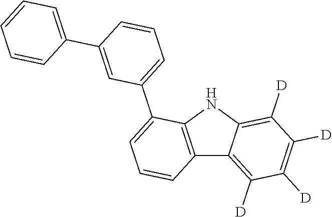 [SMILES: [2H]c1c([2H])c([2H])c2c([nH]c3c(-c4cccc(-c5ccccc5)c4)cccc32)c1[2H]] | 66 |
| b3 | 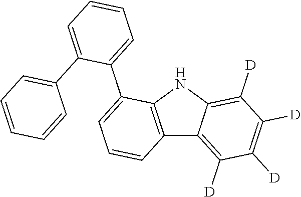 [SMILES: [2H]c1c([2H])c([2H])c2c([nH]c3c(-c4ccccc4-c4ccccc4)cccc32)c1[2H]] | 67 |
| b4 | 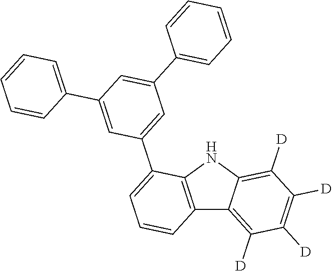 [SMILES: [2H]c1c([2H])c([2H])c2c([nH]c3c(-c4cc(-c5ccccc5)cc(-c5ccccc5)c4)cccc32)c1[2H]] | 73 |
| b5 | 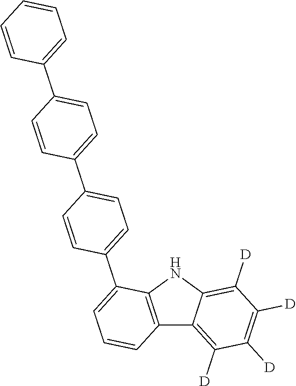 [SMILES: [2H]c1c([2H])c([2H])c2c([nH]c3c(-c4ccc(-c5ccc(-c6ccccc6)cc5)cc4)cccc32)c1[2H]] | 68 |
| c1 | 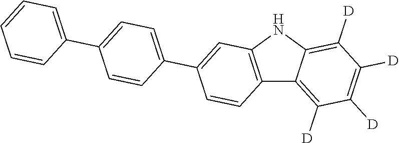 [SMILES: [2H]c1c([2H])c([2H])c2c([nH]c3cc(-c4ccc(-c5ccccc5)cc4)ccc32)c1[2H]] | 72 |
| c2 | 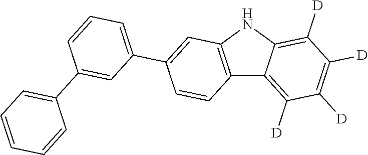 [SMILES: [2H]c1c([2H])c([2H])c2c([nH]c3cc(-c4cccc(-c5ccccc5)c4)ccc32)c1[2H]] | 74 |
| c3 | 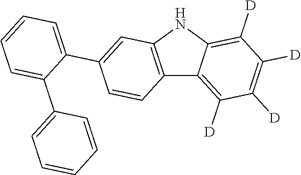 [SMILES: [2H]c1c([2H])c([2H])c2c([nH]c3cc(-c4ccccc4-c4ccccc4)ccc32)c1[2H]] | 72 |
| c4 | 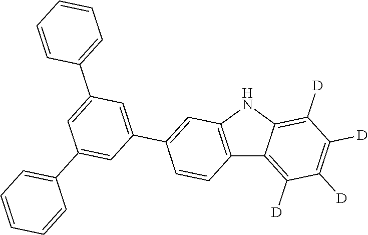 [SMILES: [2H]c1c([2H])c([2H])c2c([nH]c3cc(-c4cc(-c5ccccc5)cc(-c5ccccc5)c4)ccc32)c1[2H]] | 74 |
| d1 | 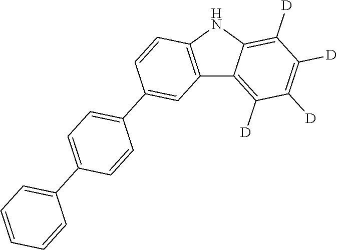 [SMILES: [2H]c1c([2H])c([2H])c2c([nH]c3ccc(-c4ccc(-c5ccccc5)cc4)cc32)c1[2H]] | 70 |
| d2 | 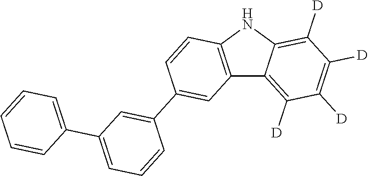 [SMILES: [2H]c1c([2H])c([2H])c2c([nH]c3ccc(-c4cccc(-c5ccccc5)c4)cc32)c1[2H]] | 62 |
| d3 | 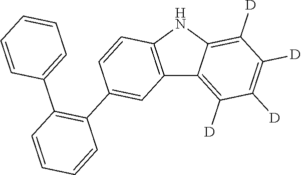 [SMILES: [2H]c1c([2H])c([2H])c2c([nH]c3ccc(-c4ccccc4-c4ccccc4)cc32)c1[2H]] | 66 |
| d4 | 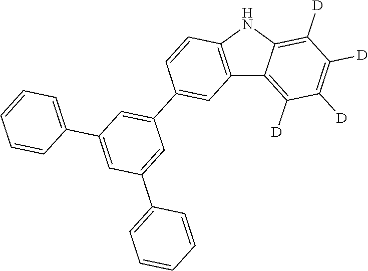 [SMILES: [2H]c1c([2H])c([2H])c2c([nH]c3ccc(-c4cc(-c5ccccc5)cc(-c5ccccc5)c4)cc32)c1[2H]] | 66 |

### Synthesis of Compound A2

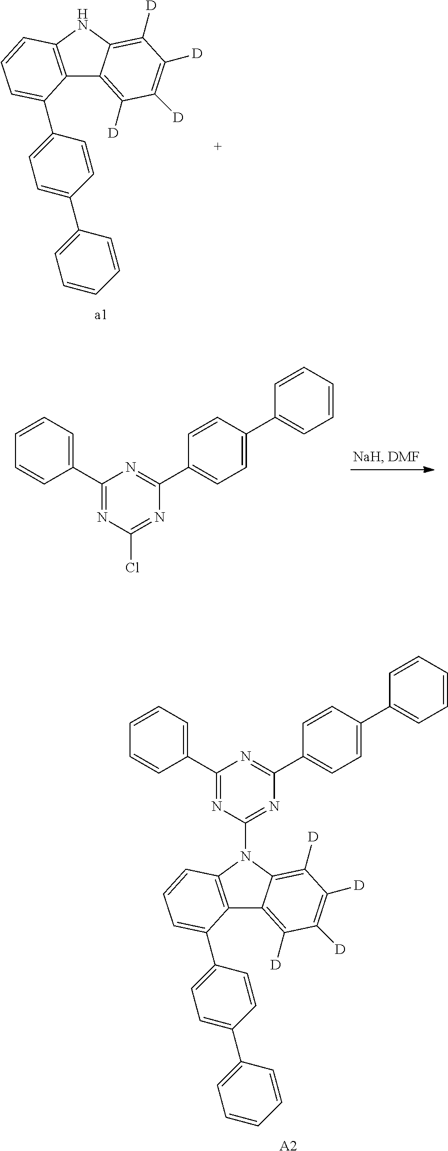

Under the protection of nitrogen, the Intermediate a1 (5.0 g; 15.5 mmol), 2-chloro-4-(biphenyl-4-yl)-6-phenyl-1,3,5-triazine (8.0 g; 23.2 mmol) and N,N-dimethylformamide (50 mL) were added into a round-bottom flask and cooled to −5° C. to 0° C. under stirring, sodium hydride (0.4 g; 18.6 mmol) was added, and stirred to react for 1 hour, and then a temperature was heated to 20° C. to 25° C., a reaction was carried out for 16 hours. The reaction was stopped, a reaction liquid was washed and then subjected to liquid separation, an organic phase was dried using anhydrous magnesium sulfate, and a solvent was removed under reduced pressure to obtain a crude product; and the crude product was purified by using a silica gel column chromatography with a dichloromethane/n-heptane mixed solvent as an eluent, and then recrystallization purification was carried out using a toluene/n-heptane mixed solvent to obtain a Compound A2 as a white solid (6.6 g; yield: 68%).

Referring to a synthesis method for Compound A2, the Intermediate a1 was replaced with Reactant E and 2-chloro-4-(biphenyl-4-yl)-6-phenyl-1,3,5-triazine was replaced with Reactant F to synthesize Compounds shown in Table 4 below:

TABLE 4

| Com- | | |
|---|---|---|
| pounds | | |
| No. | Reactant E | Reactant F |
| A9 | 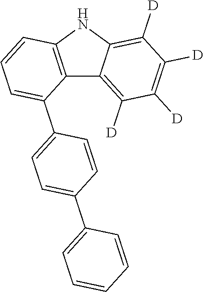 [SMILES: [2H]c1c([2H])c([2H])c2c([nH]c3cccc(-c4ccc(-c5ccccc5)cc4)c32)c1[2H]] | 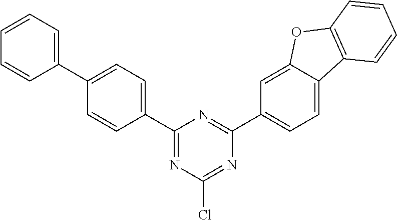 [SMILES: Clc1nc(-c2ccc(-c3ccccc3)cc2)nc(-c2ccc3c(c2)oc2ccccc23)n1] |
| A31 | 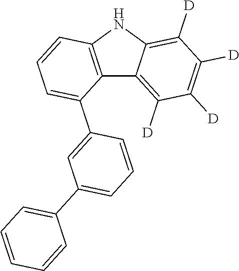 [SMILES: [2H]c1c([2H])c([2H])c2c([nH]c3cccc(-c4cccc(-c5ccccc5)c4)c32)c1[2H]] | 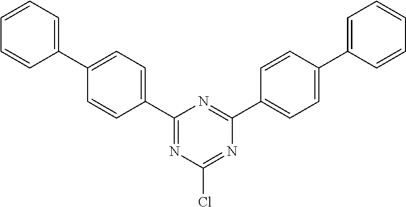 [SMILES: Clc1nc(-c2ccc(-c3ccccc3)cc2)nc(-c2ccc(-c3ccccc3)cc2)n1] |
| A55 | 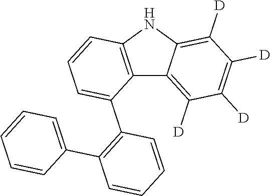 [SMILES: [2H]c1c([2H])c([2H])c2c([nH]c3cccc(-c4ccccc4-c4ccccc4)c32)c1[2H]] | 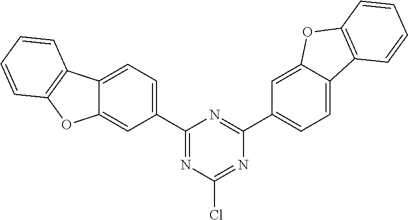 [SMILES: Clc1nc(-c2ccc3c(c2)oc2ccccc23)nc(-c2ccc3c(c2)oc2ccccc23)n1] |
| A73 | 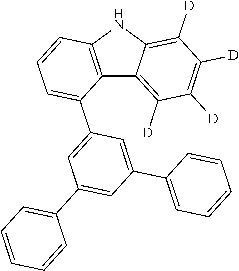 [SMILES: [2H]c1c([2H])c([2H])c2c([nH]c3cccc(-c4cc(-c5ccccc5)cc(-c5ccccc5)c4)c32)c1[2H]] | 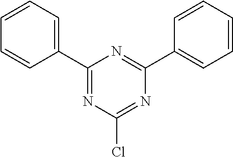 [SMILES: Clc1nc(-c2ccccc2)nc(-c2ccccc2)n1] |
| B4 | 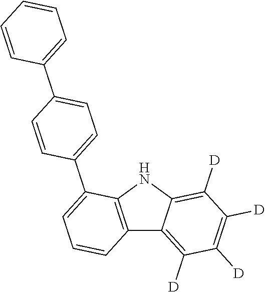 [SMILES: [2H]c1c([2H])c([2H])c2c([nH]c3c(-c4ccc(-c5ccccc5)cc4)cccc32)c1[2H]] | 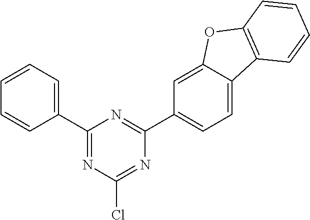 [SMILES: Clc1nc(-c2ccccc2)nc(-c2ccc3c(c2)oc2ccccc23)n1] |
| B29 | 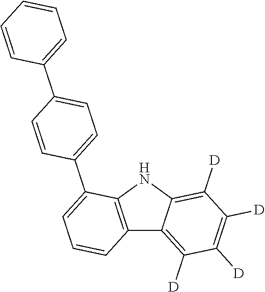 [SMILES: [2H]c1c([2H])c([2H])c2c([nH]c3c(-c4ccc(-c5ccccc5)cc4)cccc32)c1[2H]] | 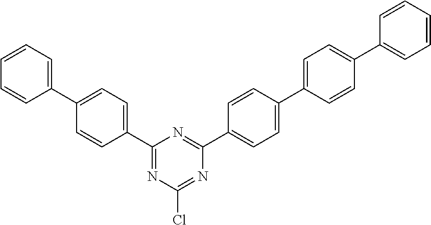 [SMILES: Clc1nc(-c2ccc(-c3ccccc3)cc2)nc(-c2ccc(-c3ccc(-c4ccccc4)cc3)cc2)n1] |
| B58 | 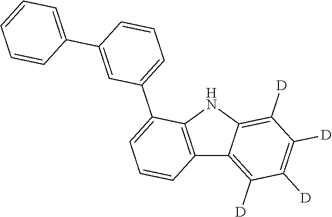 [SMILES: [2H]c1c([2H])c([2H])c2c([nH]c3c(-c4cccc(-c5ccccc5)c4)cccc32)c1[2H]] | 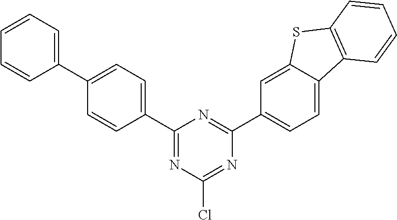 [SMILES: Clc1nc(-c2ccc(-c3ccccc3)cc2)nc(-c2ccc3c(c2)sc2ccccc23)n1] |
| C25 | 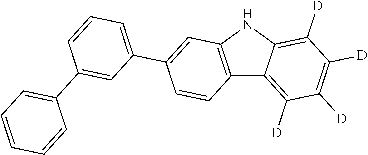 [SMILES: [2H]c1c([2H])c([2H])c2c([nH]c3cc(-c4cccc(-c5ccccc5)c4)ccc32)c1[2H]] | 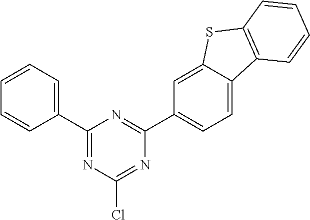 [SMILES: Clc1nc(-c2ccccc2)nc(-c2ccc3c(c2)sc2ccccc23)n1] |
| C33 | 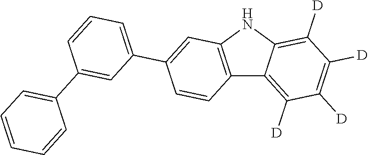 [SMILES: [2H]c1c([2H])c([2H])c2c([nH]c3cc(-c4cccc(-c5ccccc5)c4)ccc32)c1[2H]] | 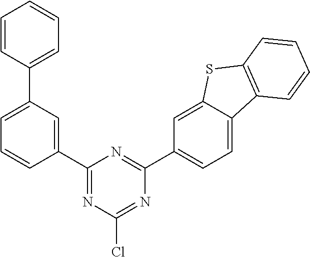 [SMILES: Clc1nc(-c2cccc(-c3ccccc3)c2)nc(-c2ccc3c(c2)sc2ccccc23)n1] |
| C42 | 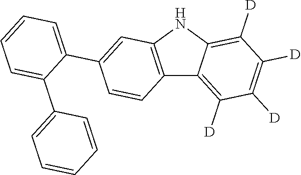 [SMILES: [2H]c1c([2H])c([2H])c2c([nH]c3cc(-c4ccccc4-c4ccccc4)ccc32)c1[2H]] | 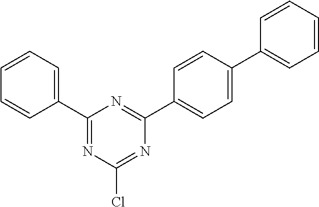 [SMILES: Clc1nc(-c2ccccc2)nc(-c2ccc(-c3ccccc3)cc2)n1] |
| D3 | 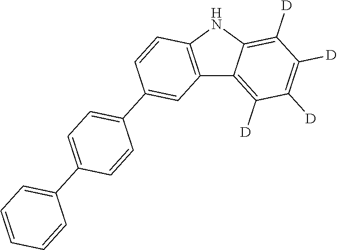 [SMILES: [2H]c1c([2H])c([2H])c2c([nH]c3ccc(-c4ccc(-c5ccccc5)cc4)cc32)c1[2H]] | 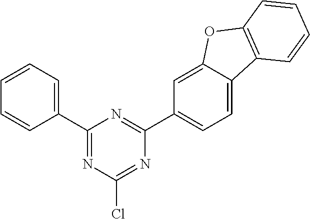 [SMILES: Clc1nc(-c2ccccc2)nc(-c2ccc3c(c2)oc2ccccc23)n1] |
| D51 | 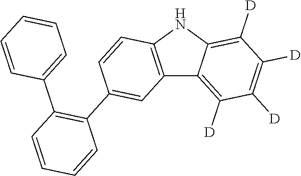 [SMILES: [2H]c1c([2H])c([2H])c2c([nH]c3ccc(-c4ccccc4-c4ccccc4)cc32)c1[2H]] | 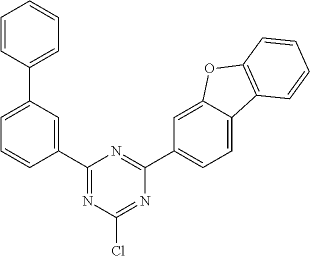 [SMILES: Clc1nc(-c2cccc(-c3ccccc3)c2)nc(-c2ccc3c(c2)oc2ccccc23)n1] |
| D61 | 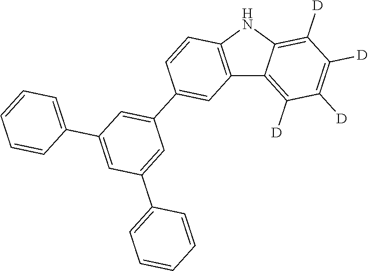 [SMILES: [2H]c1c([2H])c([2H])c2c([nH]c3ccc(-c4cc(-c5ccccc5)cc(-c5ccccc5)c4)cc32)c1[2H]] | 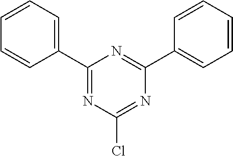 [SMILES: Clc1nc(-c2ccccc2)nc(-c2ccccc2)n1] |

| Compounds | | |
|---|---|---|
| No. | Structure | Yield (%) |
| A9 | 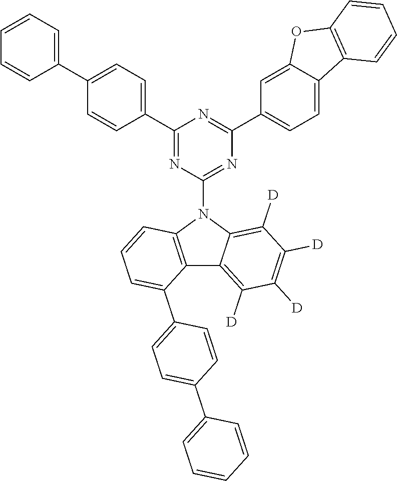 [SMILES: [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3c(c2)oc2ccccc23)n1] | 70 |
| A31 | 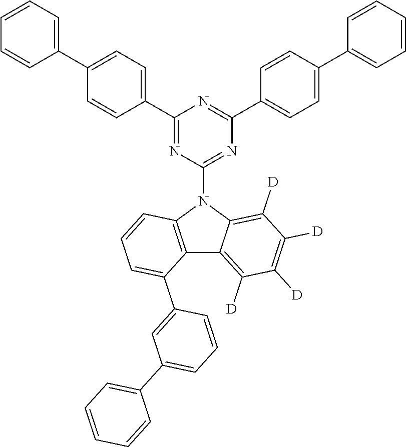 [SMILES: [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc(-c3ccccc3)cc2)n1] | 65 |
| A55 | 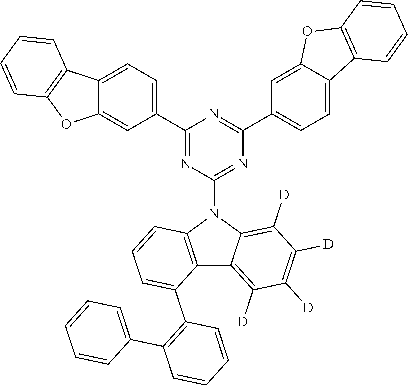 [SMILES: [2H]c1c([2H])c([2H])c2c(c1[2H])c1c(-c3ccccc3-c3ccccc3)cccc1n2-c1nc(-c2ccc3c(c2)oc2ccccc23)nc(-c2ccc3c(c2)oc2ccccc23)n1] | 72 |
| A73 | 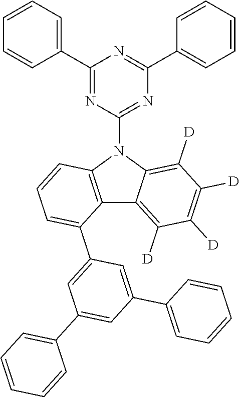 [SMILES: [2H]c1c([2H])c([2H])c2c(c1[2H])c1c(-c3cc(-c4ccccc4)cc(-c4ccccc4)c3)cccc1n2-c1nc(-c2ccccc2)nc(-c2ccccc2)n1] | 65 |
| B4 | 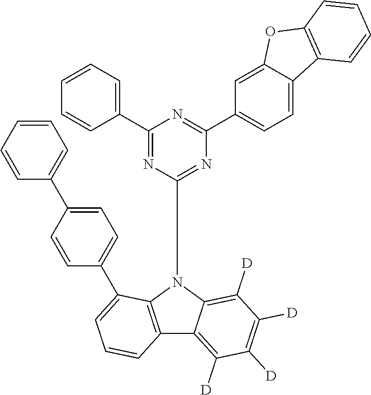 [SMILES: [2H]c1c([2H])c([2H])c2c(c1[2H])c1cccc(-c3ccc(-c4ccccc4)cc3)c1n2-c1nc(-c2ccccc2)nc(-c2ccc3c(c2)oc2ccccc23)n1] | 48 |
| B29 | 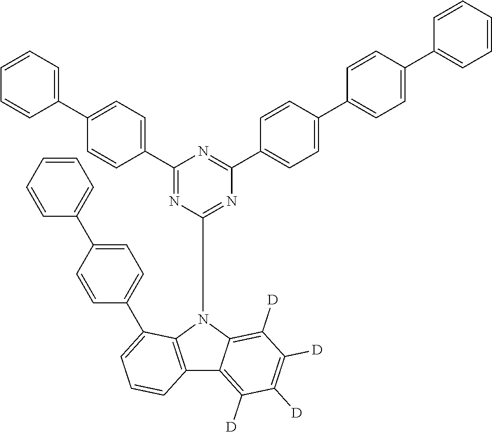 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccc(-c4ccccc4)cc3)cc2)n1] | 56 |
| B58 | 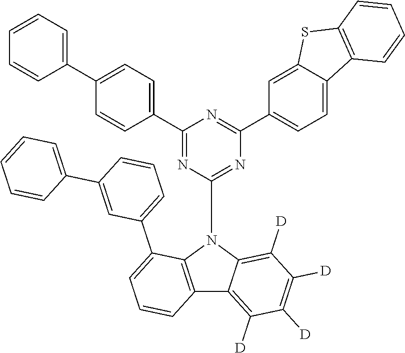 [SMILES: [2H]c1c([2H])c([2H])c2c(c1[2H])c1cccc(-c3cccc(-c4ccccc4)c3)c1n2-c1nc(-c2ccc(-c3ccccc3)cc2)nc(-c2ccc3c(c2)sc2ccccc23)n1] | 50 |
| C25 | 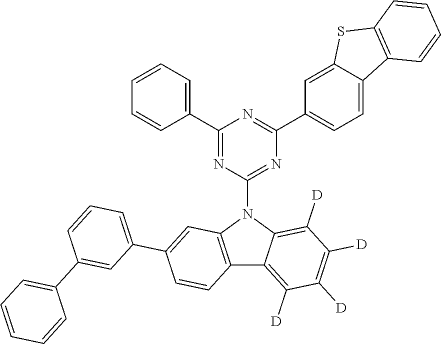 [SMILES: [2H]c1c([2H])c([2H])c2c(c1[2H])c1ccc(-c3cccc(-c4ccccc4)c3)cc1n2-c1nc(-c2ccccc2)nc(-c2ccc3c(c2)sc2ccccc23)n1] | 61 |
| C33 | 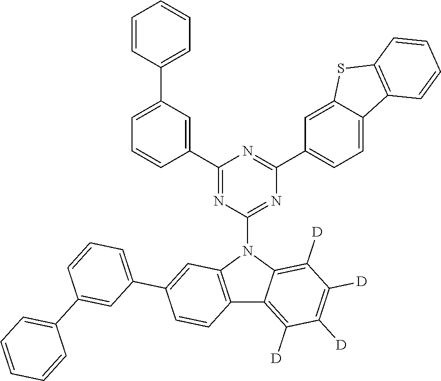 [SMILES: [2H]c1c([2H])c([2H])c2c(c1[2H])c1ccc(-c3cccc(-c4ccccc4)c3)cc1n2-c1nc(-c2cccc(-c3ccccc3)c2)nc(-c2ccc3c(c2)sc2ccccc23)n1] | 74 |
| C42 | 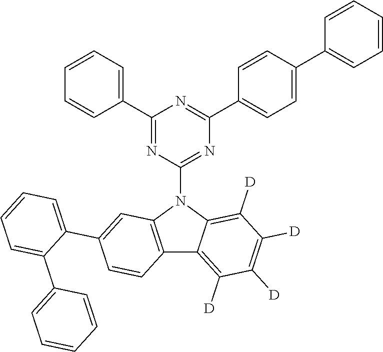 [SMILES: [2H]c1c([2H])c([2H])c2c(c1[2H])c1ccc(-c3ccccc3-c3ccccc3)cc1n2-c1nc(-c2ccccc2)nc(-c2ccc(-c3ccccc3)cc2)n1] | 64 |
| D3 | 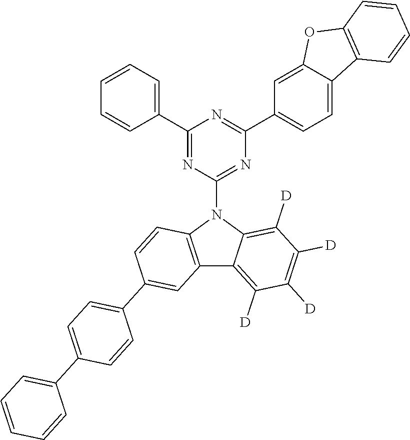 [SMILES: [2H]c1c([2H])c([2H])c2c(c1[2H])c1cc(-c3ccc(-c4ccccc4)cc3)ccc1n2-c1nc(-c2ccccc2)nc(-c2ccc3c(c2)oc2ccccc23)n1] | 59 |
| D51 | 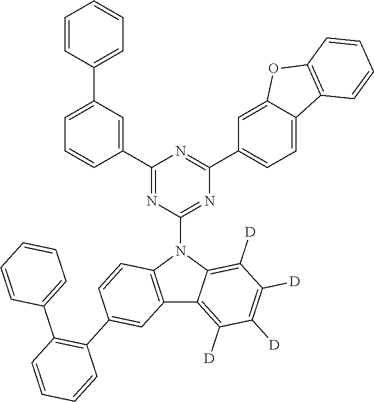 [SMILES: [2H]c1c([2H])c([2H])c2c(c1[2H])c1cc(-c3ccccc3-c3ccccc3)ccc1n2-c1nc(-c2cccc(-c3ccccc3)c2)nc(-c2ccc3c(c2)oc2ccccc23)n1] | 66 |
| D61 | 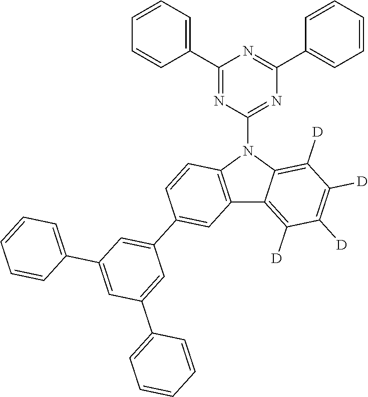 [SMILES: [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccccc2)n1] | 71 |

### Synthesis of Compound A22

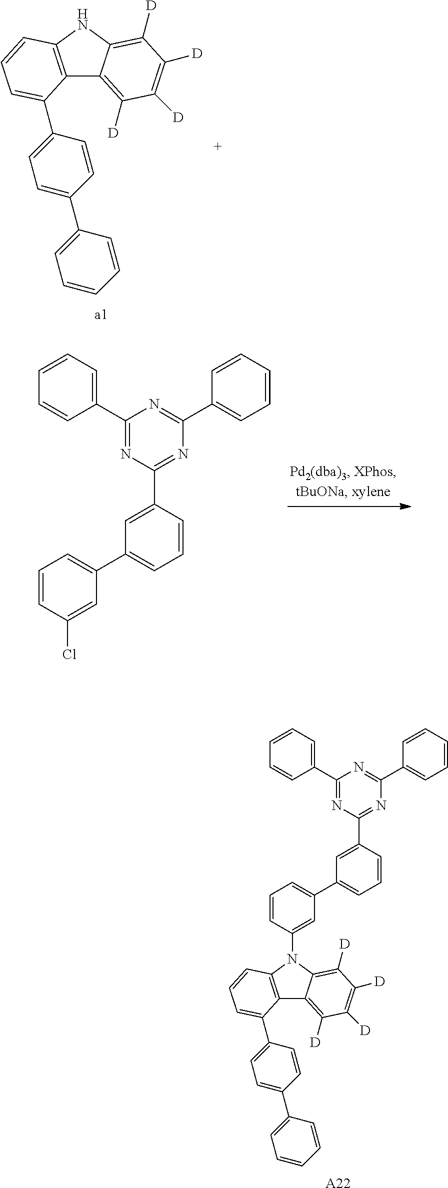

Under the protection of nitrogen, Intermediate a1 (5.0 g; 15.5 mmol), 2-(3′-chlorobiphenyl-3-yl)-4,6-diphenyl-1,3,5-triazine (6.8 g; 16.2 mmol), tris(dibenzylideneacetone) dipalladium (0.1 g; 0.2 mmol), 2-dicyclohexylphosphine-2′,4′,6′-triisopropyl biphenyl (0.1 g; 0.3 mmol), sodium tert-butoxide (2.2 g; 23.2 mmol) and xylene (50 mL) were added into a round-bottom flask, and a mixed liquid was carried out stirred at 135° C. to 140° C. to for 12 hours. The reaction was cooled to room temperature, a reaction liquid was washed and then subjected to liquid separation, an organic phase was dried using anhydrous magnesium sulfate, and a solvent was removed under reduced pressure to obtain a crude product; and the crude product was purified by using a silica gel column chromatography with a dichloromethane/n-heptane mixed solvent as an eluent, and then a product was subjected to recrystallization purification using a toluene/n-heptane mixed solvent to obtain a Compound A22 as a white solid (7.9 g; yield: 720%).

Referring to a synthesis method for Compound A22, the Intermediate a1 was replaced with Reactant G and 2-(3′-chlorobiphenyl-3-yl)-4,6-diphenyl-1,3,5-triazine was replaced with Reactant H in Table 5 below to synthesize Compounds shown in Table 5 below:

TABLE 5

| Com- | | |
|---|---|---|
| pounds | | |
| No. | Reactant G | Reactant H |
| A41 | 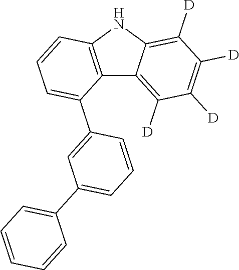 [SMILES: [2H]c1c([2H])c([2H])c2c([nH]c3cccc(-c4cccc(-c5ccccc5)c4)c32)c1[2H]] | 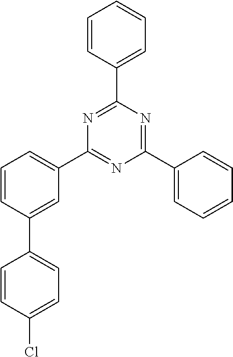 [SMILES: Clc1ccc(-c2cccc(-c3nc(-c4ccccc4)nc(-c4ccccc4)n3)c2)cc1] |
| A62 | 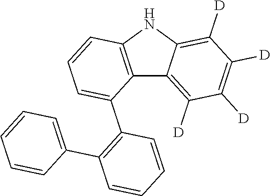 [SMILES: [2H]c1c([2H])c([2H])c2c([nH]c3cccc(-c4ccccc4-c4ccccc4)c32)c1[2H]] | 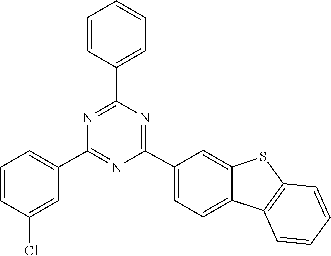 [SMILES: Clc1cccc(-c2nc(-c3ccccc3)nc(-c3ccc4c(c3)sc3ccccc34)n2)c1] |
| B19 | 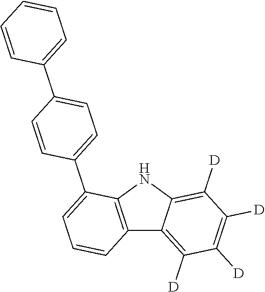 [SMILES: [2H]c1c([2H])c([2H])c2c([nH]c3c(-c4ccc(-c5ccccc5)cc4)cccc32)c1[2H]] | 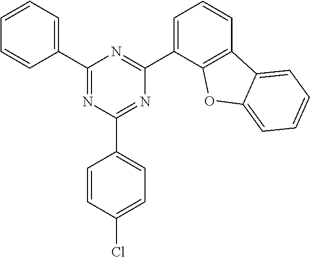 [SMILES: Clc1ccc(-c2nc(-c3ccccc3)nc(-c3cccc4c3oc3ccccc34)n2)cc1] |
| B36 | 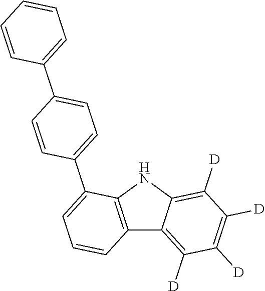 [SMILES: [2H]c1c([2H])c([2H])c2c([nH]c3c(-c4ccc(-c5ccccc5)cc4)cccc32)c1[2H]] | 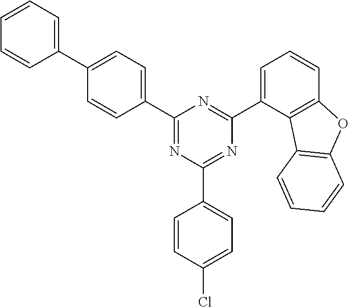 [SMILES: Clc1ccc(-c2nc(-c3ccc(-c4ccccc4)cc3)nc(-c3cccc4oc5ccccc5c34)n2)cc1] |
| B44 | 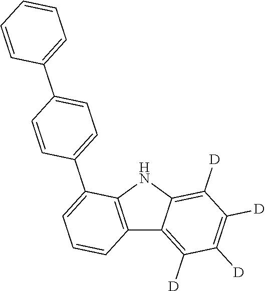 [SMILES: [2H]c1c([2H])c([2H])c2c([nH]c3c(-c4ccc(-c5ccccc5)cc4)cccc32)c1[2H]] | 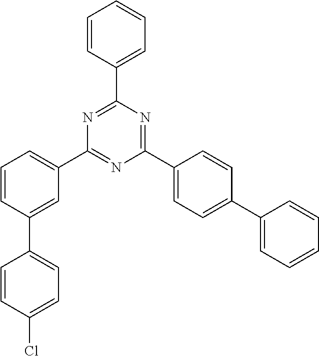 [SMILES: Clc1ccc(-c2cccc(-c3nc(-c4ccccc4)nc(-c4ccc(-c5ccccc5)cc4)n3)c2)cc1] |
| B68 | 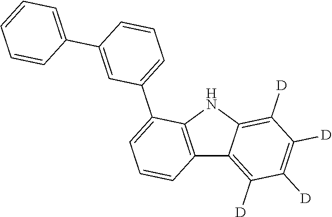 [SMILES: [2H]c1c([2H])c([2H])c2c([nH]c3c(-c4cccc(-c5ccccc5)c4)cccc32)c1[2H]] | 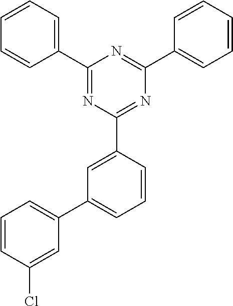 [SMILES: Clc1cccc(-c2cccc(-c3nc(-c4ccccc4)nc(-c4ccccc4)n3)c2)c1] |
| B74 | 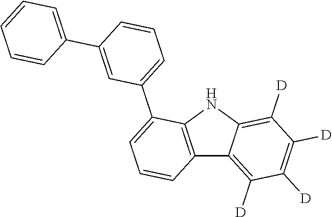 [SMILES: [2H]c1c([2H])c([2H])c2c([nH]c3c(-c4cccc(-c5ccccc5)c4)cccc32)c1[2H]] | 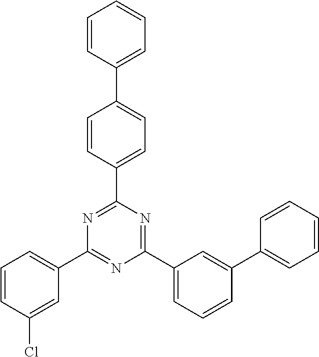 [SMILES: Clc1cccc(-c2nc(-c3ccc(-c4ccccc4)cc3)nc(-c3cccc(-c4ccccc4)c3)n2)c1] |
| B84 | 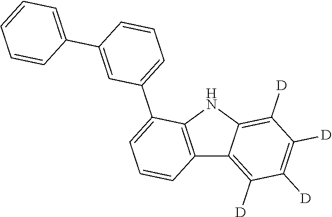 [SMILES: [2H]c1c([2H])c([2H])c2c([nH]c3c(-c4cccc(-c5ccccc5)c4)cccc32)c1[2H]] | 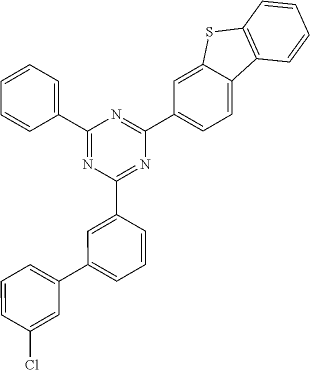 [SMILES: Clc1cccc(-c2cccc(-c3nc(-c4ccccc4)nc(-c4ccc5c(c4)sc4ccccc45)n3)c2)c1] |
| B95 | 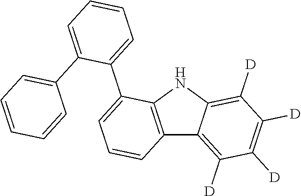 [SMILES: [2H]c1c([2H])c([2H])c2c([nH]c3c(-c4ccccc4-c4ccccc4)cccc32)c1[2H]] | 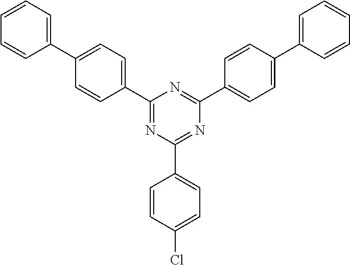 [SMILES: Clc1ccc(-c2nc(-c3ccc(-c4ccccc4)cc3)nc(-c3ccc(-c4ccccc4)cc3)n2)cc1] |
| B104 | 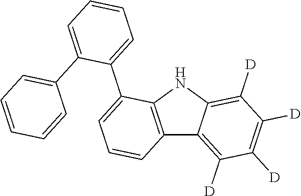 [SMILES: [2H]c1c([2H])c([2H])c2c([nH]c3c(-c4ccccc4-c4ccccc4)cccc32)c1[2H]] | 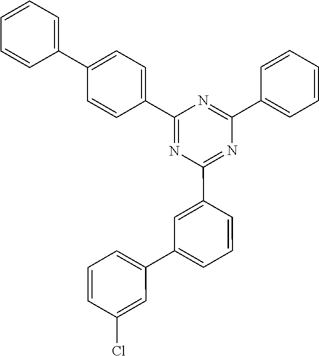 [SMILES: Clc1cccc(-c2cccc(-c3nc(-c4ccccc4)nc(-c4ccc(-c5ccccc5)cc4)n3)c2)c1] |
| B116 | 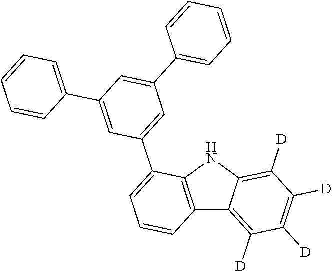 [SMILES: [2H]c1c([2H])c([2H])c2c([nH]c3c(-c4cc(-c5ccccc5)cc(-c5ccccc5)c4)cccc32)c1[2H]] | 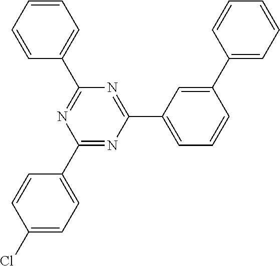 [SMILES: Clc1ccc(-c2nc(-c3ccccc3)nc(-c3cccc(-c4ccccc4)c3)n2)cc1] |
| B128 | 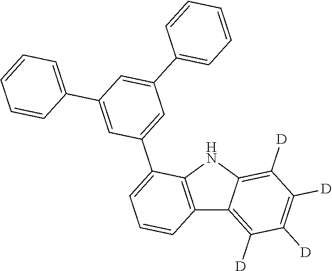 [SMILES: [2H]c1c([2H])c([2H])c2c([nH]c3c(-c4cc(-c5ccccc5)cc(-c5ccccc5)c4)cccc32)c1[2H]] | 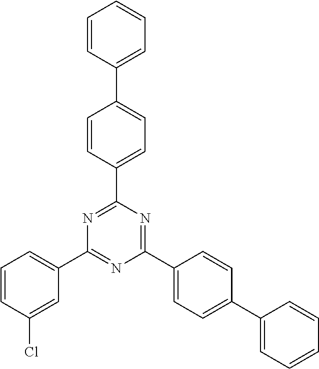 [SMILES: Clc1cccc(-c2nc(-c3ccc(-c4ccccc4)cc3)nc(-c3ccc(-c4ccccc4)cc3)n2)c1] |
| B133 | 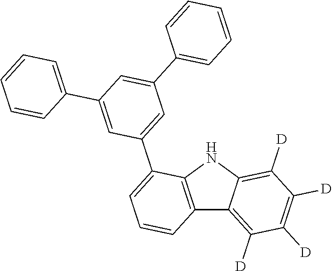 [SMILES: [2H]c1c([2H])c([2H])c2c([nH]c3c(-c4cc(-c5ccccc5)cc(-c5ccccc5)c4)cccc32)c1[2H]] | 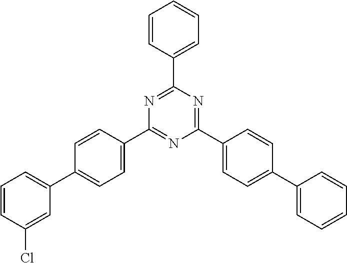 [SMILES: Clc1cccc(-c2ccc(-c3nc(-c4ccccc4)nc(-c4ccc(-c5ccccc5)cc4)n3)cc2)c1] |
| B143 | 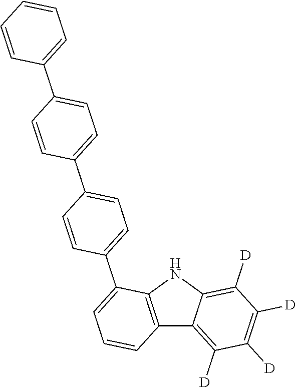 [SMILES: [2H]c1c([2H])c([2H])c2c([nH]c3c(-c4ccc(-c5ccc(-c6ccccc6)cc5)cc4)cccc32)c1[2H]] | 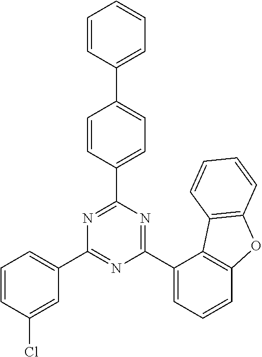 [SMILES: Clc1cccc(-c2nc(-c3ccc(-c4ccccc4)cc3)nc(-c3cccc4oc5ccccc5c34)n2)c1] |
| C4 | 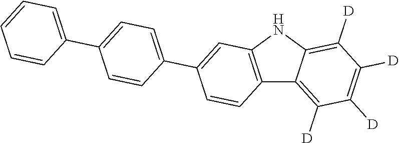 [SMILES: [2H]c1c([2H])c([2H])c2c([nH]c3cc(-c4ccc(-c5ccccc5)cc4)ccc32)c1[2H]] | 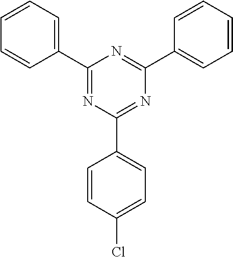 [SMILES: Clc1ccc(-c2nc(-c3ccccc3)nc(-c3ccccc3)n2)cc1] |
| C11 | 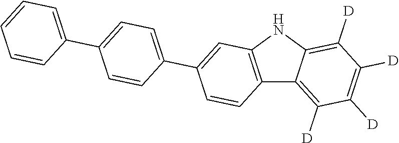 [SMILES: [2H]c1c([2H])c([2H])c2c([nH]c3cc(-c4ccc(-c5ccccc5)cc4)ccc32)c1[2H]] | 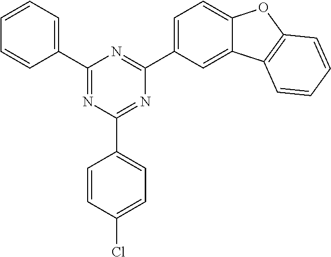 [SMILES: Clc1ccc(-c2nc(-c3ccccc3)nc(-c3ccc4oc5ccccc5c4c3)n2)cc1] |
| C14 | 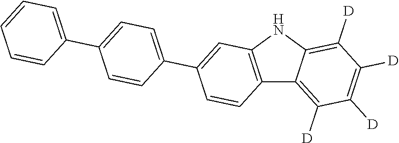 [SMILES: [2H]c1c([2H])c([2H])c2c([nH]c3cc(-c4ccc(-c5ccccc5)cc4)ccc32)c1[2H]] | 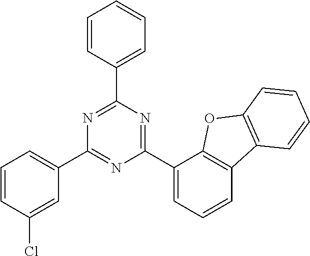 [SMILES: Clc1cccc(-c2nc(-c3ccccc3)nc(-c3cccc4c3oc3ccccc34)n2)c1] |
| C52 | 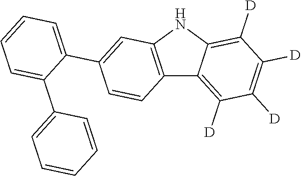 [SMILES: [2H]c1c([2H])c([2H])c2c([nH]c3cc(-c4ccccc4-c4ccccc4)ccc32)c1[2H]] | 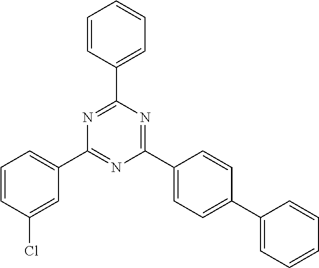 [SMILES: Clc1cccc(-c2nc(-c3ccccc3)nc(-c3ccc(-c4ccccc4)cc3)n2)c1] |
| C60 | 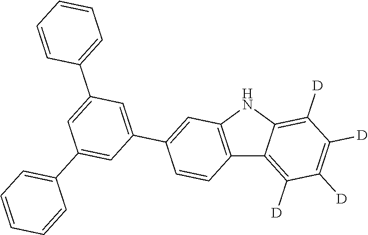 [SMILES: [2H]c1c([2H])c([2H])c2c([nH]c3cc(-c4cc(-c5ccccc5)cc(-c5ccccc5)c4)ccc32)c1[2H]] | 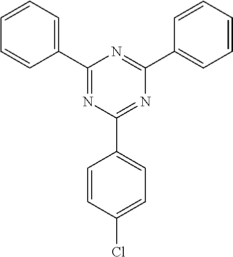 [SMILES: Clc1ccc(-c2nc(-c3ccccc3)nc(-c3ccccc3)n2)cc1] |
| D10 | 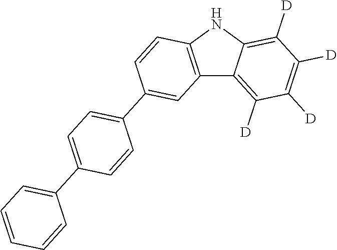 [SMILES: [2H]c1c([2H])c([2H])c2c([nH]c3ccc(-c4ccc(-c5ccccc5)cc4)cc32)c1[2H]] | 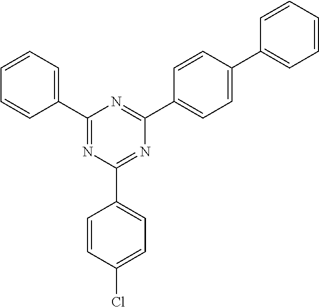 [SMILES: Clc1ccc(-c2nc(-c3ccccc3)nc(-c3ccc(-c4ccccc4)cc3)n2)cc1] |
| D26 | 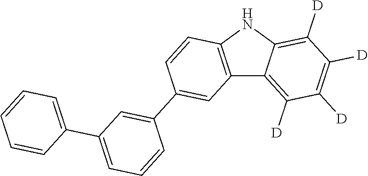 [SMILES: [2H]c1c([2H])c([2H])c2c([nH]c3ccc(-c4cccc(-c5ccccc5)c4)cc32)c1[2H]] | 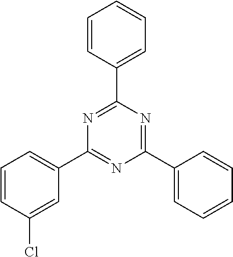 [SMILES: Clc1cccc(-c2nc(-c3ccccc3)nc(-c3ccccc3)n2)c1] |
| D33 | 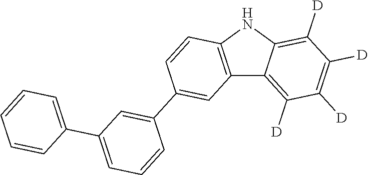 [SMILES: [2H]c1c([2H])c([2H])c2c([nH]c3ccc(-c4cccc(-c5ccccc5)c4)cc32)c1[2H]] | 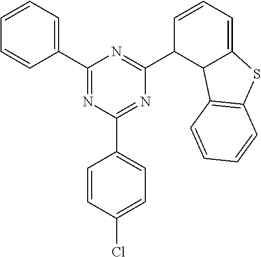 [SMILES: Clc1ccc(-c2nc(-c3ccccc3)nc(C3C=CC=C4Sc5ccccc5C43)n2)cc1] |

| Compounds | | |
|---|---|---|
| No. | Structure | Yield (%) |
| A41 | 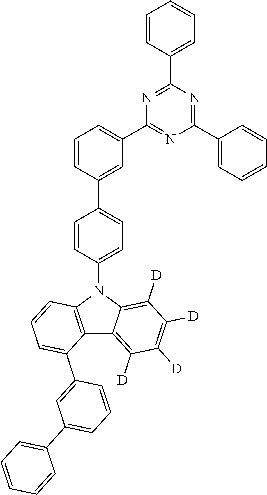 [SMILES: [2H]c1c([2H])c([2H])c2c(c1[2H])c1c(-c3cccc(-c4ccccc4)c3)cccc1n2-c1ccc(-c2cccc(-c3nc(-c4ccccc4)nc(-c4ccccc4)n3)c2)cc1] | 61 |
| A62 | 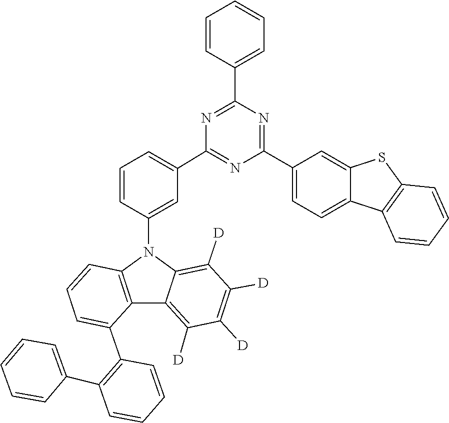 [SMILES: [2H]c1c([2H])c([2H])c2c(c1[2H])c1c(-c3ccccc3-c3ccccc3)cccc1n2-c1cccc(-c2nc(-c3ccccc3)nc(-c3ccc4c(c3)sc3ccccc34)n2)c1] | 70 |
| B19 | 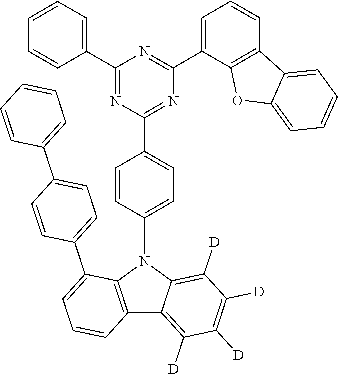 [SMILES: [2H]c1c([2H])c([2H])c2c(c1[2H])c1cccc(-c3ccc(-c4ccccc4)cc3)c1n2-c1ccc(-c2nc(-c3ccccc3)nc(-c3cccc4c3oc3ccccc34)n2)cc1] | 47 |
| B36 | 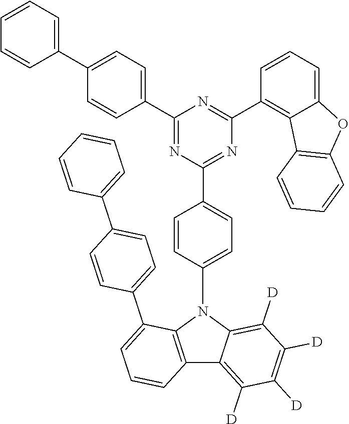 [SMILES: [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3cccc4oc5ccccc5c34)n2)cc1] | 48 |
| B44 | 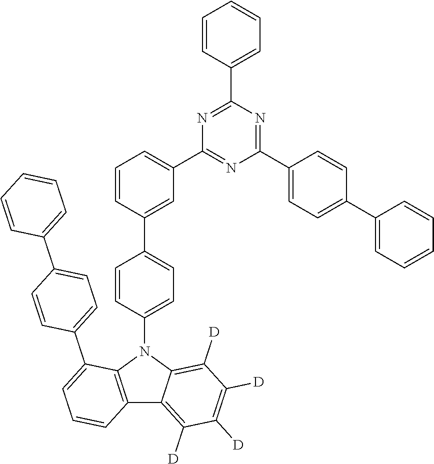 [SMILES: [2H]c1c([2H])c([2H])c2c(c1[2H])c1cccc(-c3ccc(-c4ccccc4)cc3)c1n2-c1ccc(-c2cccc(-c3nc(-c4ccccc4)nc(-c4ccc(-c5ccccc5)cc4)n3)c2)cc1] | 58 |
| B68 | 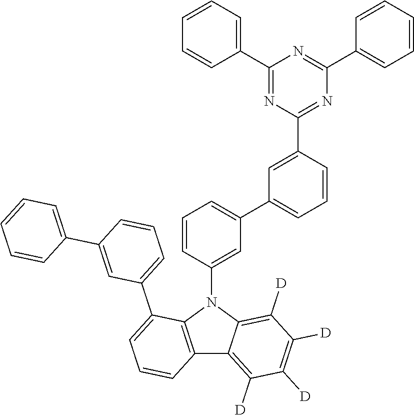 [SMILES: [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccccc4)n3)c2)c1] | 50 |
| B74 | 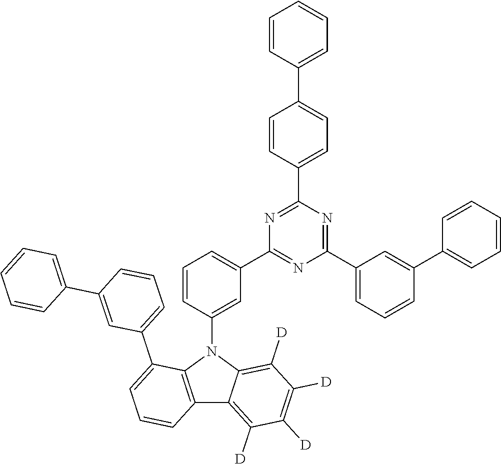 [SMILES: [2H]c1c([2H])c([2H])c2c(c1[2H])c1cccc(-c3cccc(-c4ccccc4)c3)c1n2-c1cccc(-c2nc(-c3ccc(-c4ccccc4)cc3)nc(-c3cccc(-c4ccccc4)c3)n2)c1] | 55 |
| B84 | 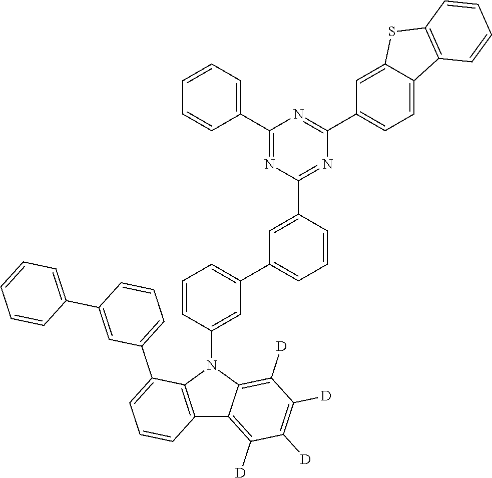 [SMILES: [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccc5c(c4)sc4ccccc45)n3)c2)c1] | 46 |
| B95 | 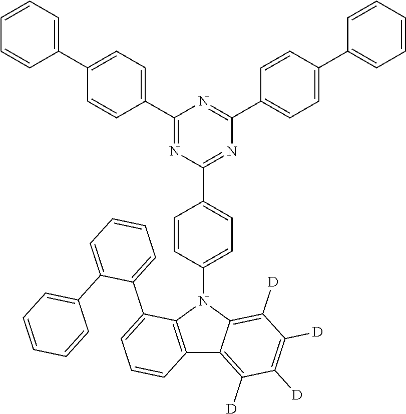 [SMILES: [2H]c1c([2H])c([2H])c2c(c1[2H])c1cccc(-c3ccccc3-c3ccccc3)c1n2-c1ccc(-c2nc(-c3ccc(-c4ccccc4)cc3)nc(-c3ccc(-c4ccccc4)cc3)n2)cc1] | 42 |
| B104 | 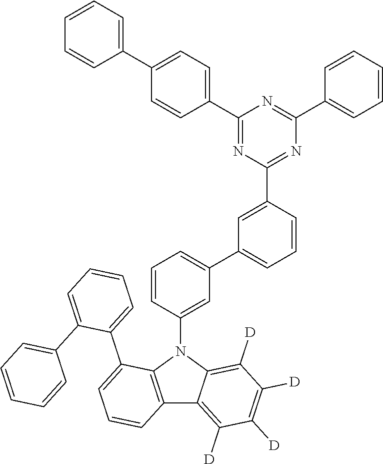 [SMILES: [2H]c1c([2H])c([2H])c2c(c1[2H])c1cccc(-c3ccccc3-c3ccccc3)c1n2-c1cccc(-c2cccc(-c3nc(-c4ccccc4)nc(-c4ccc(-c5ccccc5)cc4)n3)c2)c1] | 37 |
| B116 | 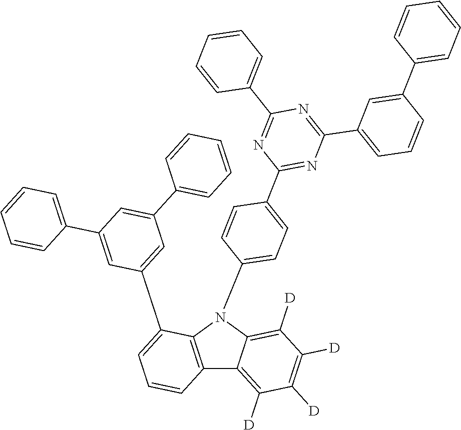 [SMILES: [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3cccc(-c4ccccc4)c3)n2)cc1] | 38 |
| B128 | 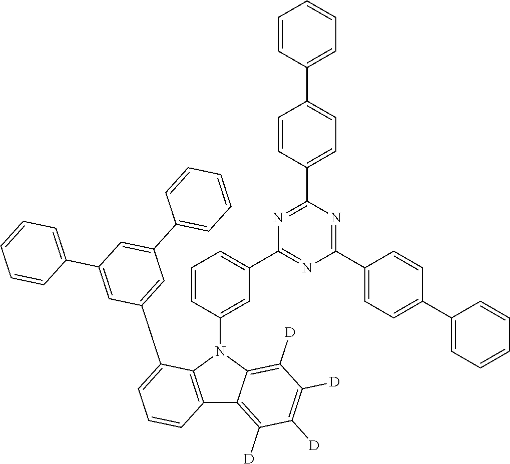 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3ccc(-c4ccccc4)cc3)n2)c1] | 45 |
| B133 | 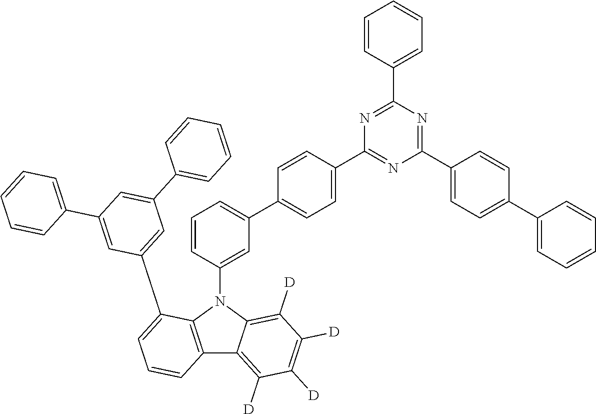 [SMILES: [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccc(-c5ccccc5)cc4)n3)cc2)c1] | 51 |
| B143 | 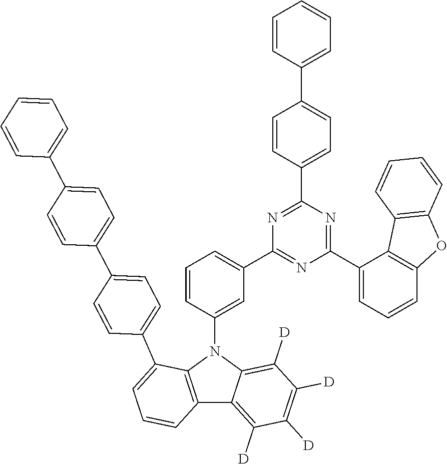 [SMILES: [2H]c1c([2H])c([2H])c2c(c1[2H])c1cccc(-c3ccc(-c4ccc(-c5ccccc5)cc4)cc3)c1n2-c1cccc(-c2nc(-c3ccc(-c4ccccc4)cc3)nc(-c3cccc4oc5ccccc5c34)n2)c1] | 39 |
| C4 | 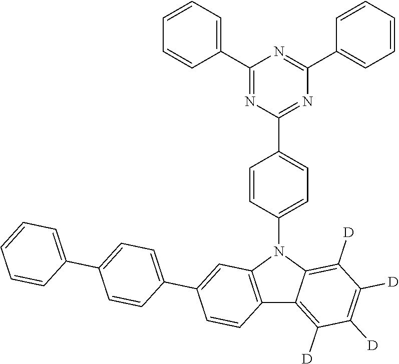 [SMILES: [2H]c1c([2H])c([2H])c2c(c1[2H])c1ccc(-c3ccc(-c4ccccc4)cc3)cc1n2-c1ccc(-c2nc(-c3ccccc3)nc(-c3ccccc3)n2)cc1] | 61 |
| C11 | 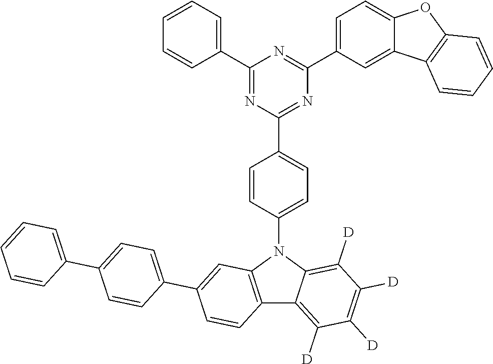 [SMILES: [2H]c1c([2H])c([2H])c2c(c1[2H])c1ccc(-c3ccc(-c4ccccc4)cc3)cc1n2-c1ccc(-c2nc(-c3ccccc3)nc(-c3ccc4oc5ccccc5c4c3)n2)cc1] | 76 |
| C14 | 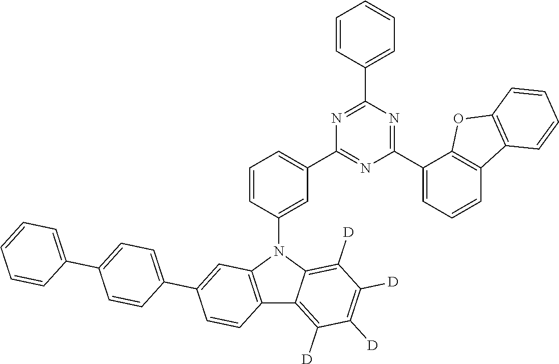 [SMILES: [2H]c1c([2H])c([2H])c2c(c1[2H])c1ccc(-c3ccc(-c4ccccc4)cc3)cc1n2-c1cccc(-c2nc(-c3ccccc3)nc(-c3cccc4c3oc3ccccc34)n2)c1] | 68 |
| C52 | 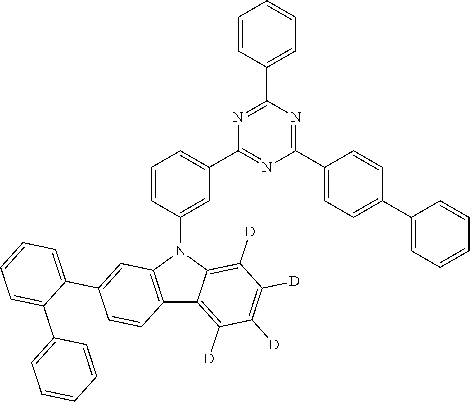 [SMILES: [2H]c1c([2H])c([2H])c2c(c1[2H])c1ccc(-c3ccccc3-c3ccccc3)cc1n2-c1cccc(-c2nc(-c3ccccc3)nc(-c3ccc(-c4ccccc4)cc3)n2)c1] | 62 |
| C60 | 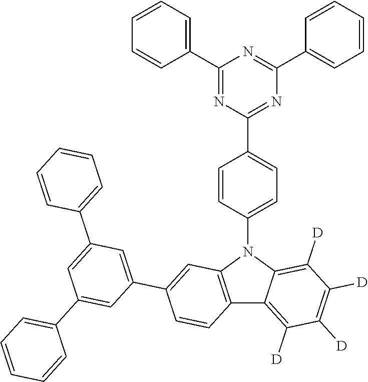 [SMILES: [2H]c1c([2H])c([2H])c2c(c1[2H])c1ccc(-c3cc(-c4ccccc4)cc(-c4ccccc4)c3)cc1n2-c1ccc(-c2nc(-c3ccccc3)nc(-c3ccccc3)n2)cc1] | 52 |
| D10 | 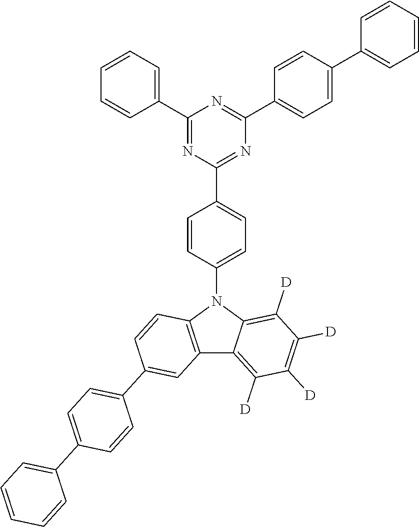 [SMILES: [2H]c1c([2H])c([2H])c2c(c1[2H])c1cc(-c3ccc(-c4ccccc4)cc3)ccc1n2-c1ccc(-c2nc(-c3ccccc3)nc(-c3ccc(-c4ccccc4)cc3)n2)cc1] | 67 |
| D26 | 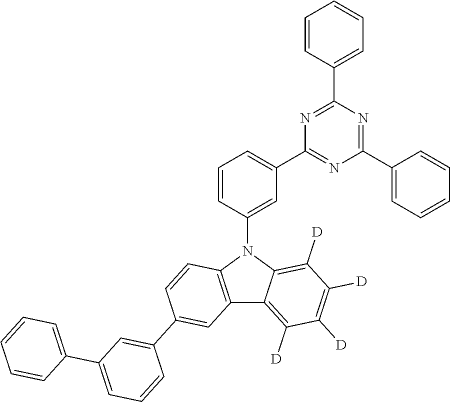 [SMILES: [2H]c1c([2H])c([2H])c2c(c1[2H])c1cc(-c3cccc(-c4ccccc4)c3)ccc1n2-c1cccc(-c2nc(-c3ccccc3)nc(-c3ccccc3)n2)c1] | 71 |
| D33 | 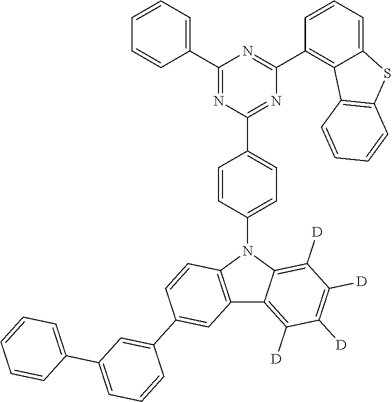 [SMILES: [2H]c1c([2H])c([2H])c2c(c1[2H])c1cc(-c3cccc(-c4ccccc4)c3)ccc1n2-c1ccc(-c2nc(-c3ccccc3)nc(-c3cccc4sc5ccccc5c34)n2)cc1] | 74 |

The mass spectrum data of part of the compounds were as shown in Table 6 below:

TABLE 6

| Compound | Mass spectrum data |
|---|---|
| Compound A2 | m/z = 631.3(M + H)⁺ |
| Compound A9 | m/z = 721.3(M + H)⁺ |
| Compound A22 | m/z = 707.3(M + H)⁺ |
| Compound A31 | m/z = 707.3(M + H)⁺ |
| Compound A41 | m/z = 707.3(M + H)⁺ |
| Compound A55 | m/z = 735.3(M + H)⁺ |
| Compound A62 | m/z = 737.3(M + H)⁺ |
| Compound A73 | m/z = 631.3(M + H)⁺ |
| Compound B4 | m/z = 645.3(M + H)⁺ |
| Compound B19 | m/z = 721.3(M + H)⁺ |
| Compound B29 | m/z = 783.3(M + H)⁺ |
| Compound B36 | m/z = 797.3(M + H)⁺ |
| Compound B44 | m/z = 783.3(M + H)⁺ |
| Compound B58 | m/z = 737.3(M + H)⁺ |
| Compound B68 | m/z = 707.3(M + H)⁺ |
| Compound B74 | m/z = 783.3(M + H)⁺ |
| Compound B84 | m/z = 813.3(M + H)⁺ |
| Compound B95 | m/z = 783.3(M + H)⁺ |
| Compound B104 | m/z = 783.3(M + H)⁺ |
| Compound B116 | m/z = 783.3(M + H)⁺ |
| Compound B128 | m/z = 859.4(M + H)⁺ |
| Compound B133 | m/z = 859.4(M + H)⁺ |
| Compound B143 | m/z = 873.4(M + H)⁺ |
| Compound C4 | m/z = 631.3(M + H)⁺ |
| Compound C11 | m/z = 721.3(M + H)⁺ |
| Compound C14 | m/z = 721.3(M + H)⁺ |
| Compound C25 | m/z = 661.2(M + H)⁺ |
| Compound C33 | m/z = 737.3(M + H)⁺ |
| Compound C42 | m/z = 631.3(M + H)⁺ |
| Compound C52 | m/z = 707.3(M + H)⁺ |
| Compound C60 | m/z = 707.3(M + H)⁺ |
| Compound D3 | m/z = 645.3(M + H)⁺ |
| Compound D10 | m/z = 707.3(M + H)⁺ |
| Compound D26 | m/z = 631.3(M + H)⁺ |
| Compound D33 | m/z = 737.3(M + H)⁺ |
| Compound D51 | m/z = 721.3(M + H)⁺ |
| Compound D61 | m/z = 631.3(M + H)⁺ |

The NMR data of part of the compounds were as shown in Table 7 below:

TABLE 7

| Compound | Nuclear magnetic data |
|---|---|
| Compound A2 | ¹H NMR (CD₂Cl₂, 400 MHz): 9.19 (d, 1H), 8.83 (d, |
| | 2H), 8.78 (d, 2H), 7.85 (d, 2H), 7.75 (d, 2H), |
| | 7.72-7.55 (m, 10H), 7.51-7.43 (m, 6H), 7.29 (d, 1H). |

### Preparation and Performance Evaluation of Organic Electroluminescent Device

### Example 1

### Green Organic Electroluminescent Device

Pretreatment of an anode was carried out first through the following process: on ITO/Ag/ITO substrates with thicknesses being 90 Å/1000 Å/100 Å respectively, surface treatment was carried out using ultraviolet ozone and O₂:N₂ plasma to increase a work function of the anode, and the surfaces of the ITO substrates were cleaned using an organic solvent to remove impurities and oil stains on the surfaces of the ITO substrates.

On an experiment substrate (anode), PD:HT-23 was subjected to co-evaporation with an evaporation rate ratio of 3%: 97% to form a hole injection layer (HIL) with a thickness of 100 Å, and then vacuum evaporation of HT-23 was carried out on the hole injection layer to form a hole transport layer with a thickness of 1260 Å.

HT-24 was evaporated on the hole transport layer to form a hole auxiliary layer with a thickness of 340 Å.

On the hole auxiliary layer, a composition GH-1-1 and GD were subjected to co-evaporation with an evaporation rate ratio of 100%: 10% to form an organic light-emitting layer (green organic light-emitting layer) with a thickness of 320 Å.

ET-01 and LiQ were mixed with a weight ratio of 1:1 and evaporated to form an electron transport layer with a thickness of 340 Å, Yb was evaporated on the electron transport layer to form an electron injection layer with a thickness of 12 Å, and magnesium and silver were co-evaporated on the electron injection layer with an evaporation ratio of 1:9 to form a cathode with a thickness of 120 Å.

In addition, CP-01 was evaporated on the above cathode to form an organic capping layer (CPL) with a thickness of 650 Å, and thus preparation of the organic light-emitting device was achieved.

### Examples 2 to 39

When the organic light-emitting layer was formed, the composition GH-1-1 in Example 1 was replaced with GH-X-Y type host material compositions shown in Table 8, and the organic electroluminescent device was manufactured using a method the same as that in Example 1.

### Comparative Examples 1 to 6

In addition to using GH-X-Y for forming the organic light-emitting layer, the organic electroluminescent device was prepared using a method the same as that in Device Example 1.

In the above examples and comparative examples, the host material compositions GH-X-Y used are obtained by uniformly mixing first compounds and second compounds in Table 8 below respectively, and specific constitutions are as shown in Table 8. A mass ratio refers to a ratio of mass percentage contents of the first compounds to the second compounds shown in the table. Taking a composition GH-1-1 as an example, it can be seen in combination with Table 8 that, GH-1-1 is formed by mixing a compound A2 and a compound 76 according to a mass ratio of 40:60, and for another example, a host material GH-D1-1 in Comparative Example 1 is formed by mixing a compound I and a compound 76 according to a mass ratio of 40:60.

Other second compounds adopted as shown below, a compound 76 is obtained according to the recordings in the patent literature JP3139321B2; a compound 77 is obtained according to the patent literature CN103518271B; a compound 78 is obtained according to the patent literature CN103430344B; a compound 79 is obtained according to the recordings in the patent literature CN104205393B; and a compound 80 is obtained according to the recordings in the patent literature US20140231779 Å1.

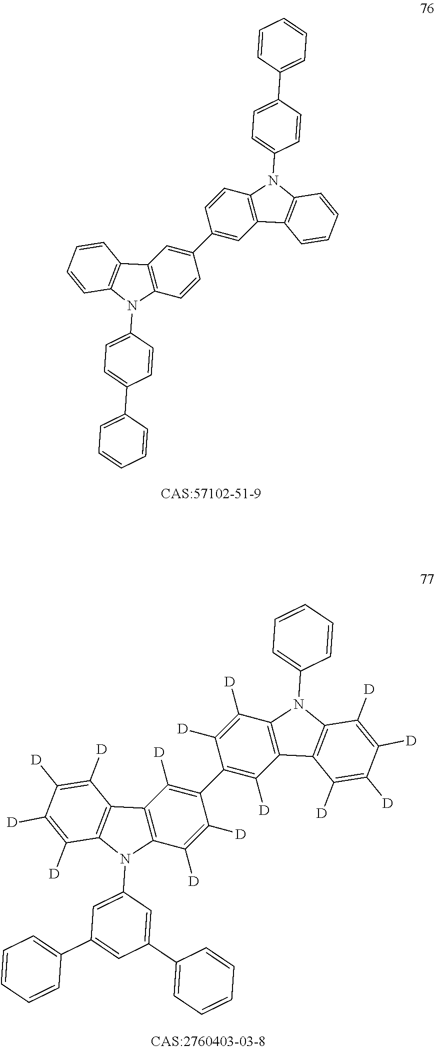 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1ccccc1]

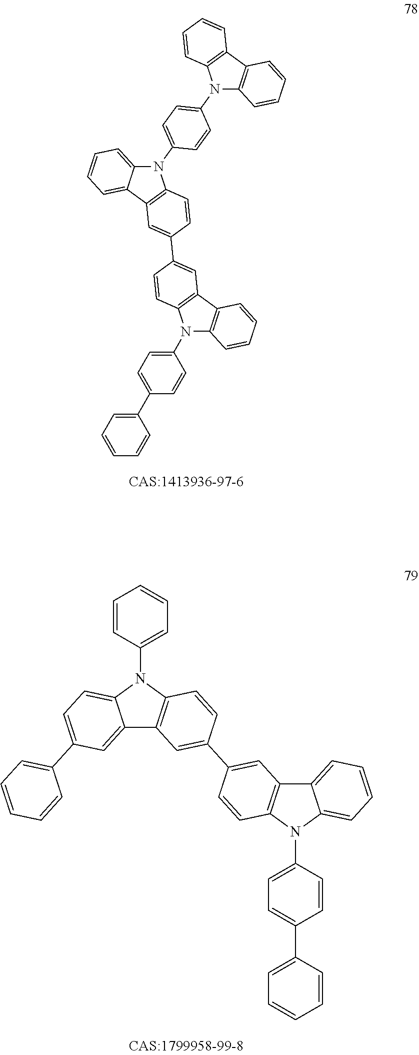 [SMILES: c1ccc(-c2ccc(-n3c4ccccc4c4cc(-c5ccc6c(c5)c5ccccc5n6-c5ccc(-n6c7ccccc7c7ccccc76)cc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

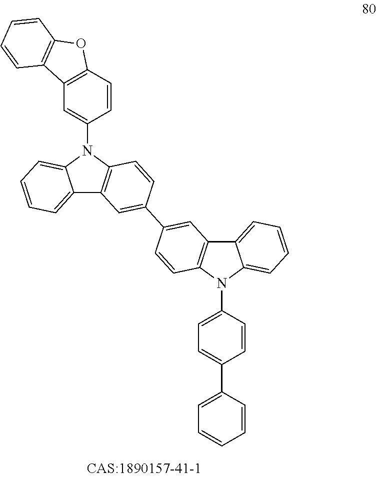 [SMILES: c1ccc(-c2ccc(-n3c4ccccc4c4cc(-c5ccc6c(c5)c5ccccc5n6-c5ccc6oc7ccccc7c6c5)ccc43)cc2)cc1]

Structural formulas of other main materials adopted in Examples 1 to 39 and Comparative Examples 1 to 6 are as shown below:

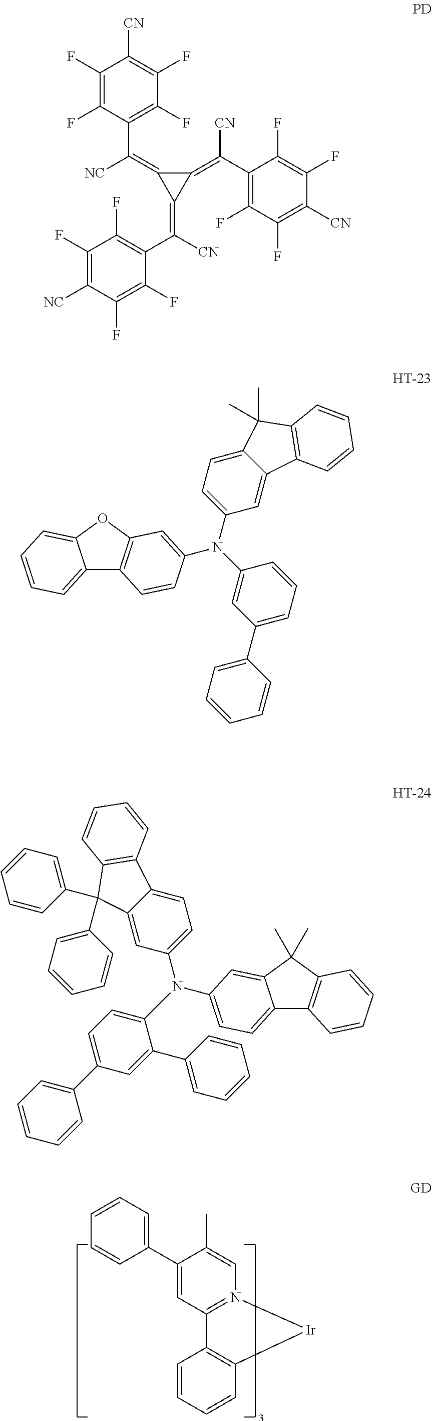

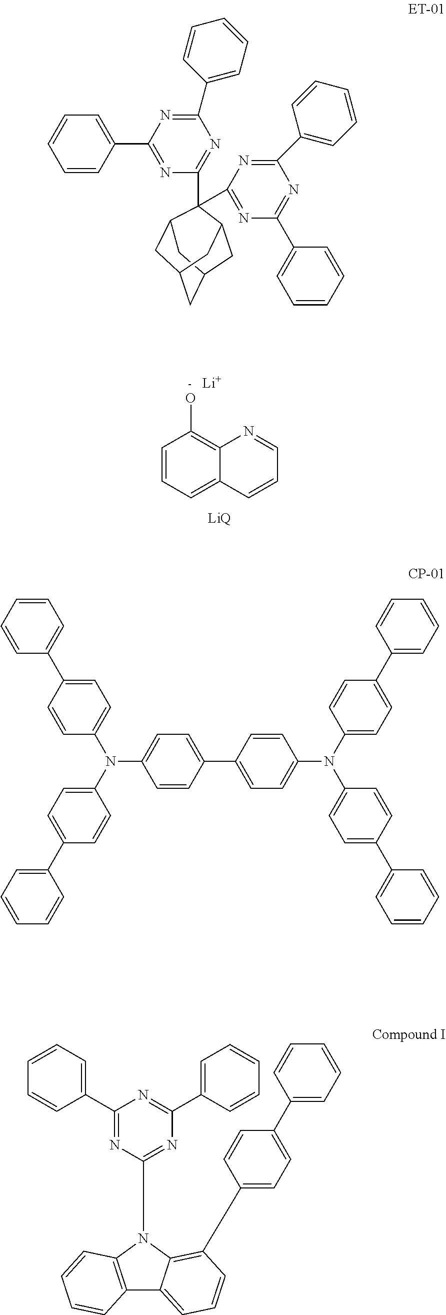

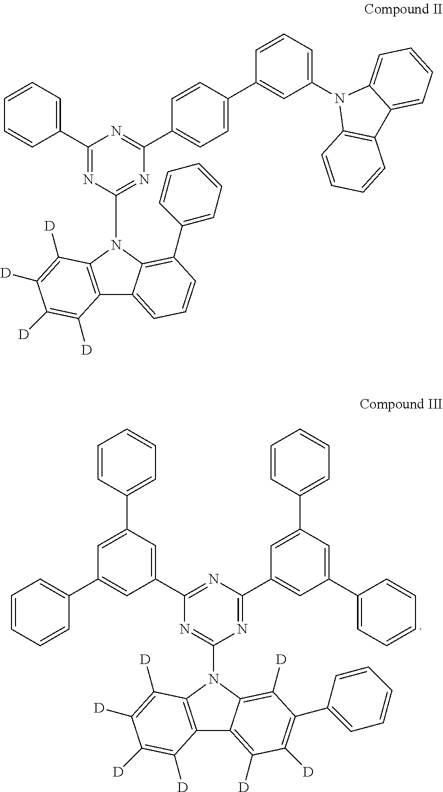 [SMILES: [2H]c1c([2H])c([2H])c2c(c1[2H])c1cccc(-c3ccccc3)c1n2-c1nc(-c2ccccc2)nc(-c2ccc(-c3cccc(-n4c5ccccc5c5ccccc54)c3)cc2)n1 ; [2H]c1c([2H])c([2H])c2c(c1[2H])c1c([2H])c([2H])c(-c3ccccc3)c([2H])c1n2-c1nc(-c2cc(-c3ccccc3)cc(-c3ccccc3)c2)nc(-c2cc(-c3ccccc3)cc(-c3ccccc3)c2)n1 ; "."]

For the organic electroluminescent devices prepared above, IVL performance of the devices are tested specifically under the condition of 10 mA/cm², the T95 lifetimes of the devices are tested under the condition of 30 mA/cm², and test results are shown in Table 8.

TABLE 8

| | Composition GH-X-Y | | | | | | | |
|---|---|---|---|---|---|---|---|---|
| | | | Mass ratio | | | | | |
| | | | of first | | | | External | |
| | | | compounds | | Luminous | Chromaticity | quantum | T95(h) |
| | First | Second | to second | Voltage | efficiency | coordinates | efficiency | @30 |
| Example | compound | compound | compounds | (V) | (Cd/A) | CIEx, CIEy | EQE(%) | mA/cm² |
| Example 1 | GH-1-1 | | | 3.72 | 118.10 | 0.23, 0.72 | 28.34 | 234 |
| | Compound | Compound | 40:60 | | | | | |
| | A2 | 76 | | | | | | |
| Example 2 | GH-2-1 | | | 3.74 | 115.21 | 0.23, 0.72 | 27.65 | 249 |
| | Compound | Compound | 40:60 | | | | | |
| | A9 | 77 | | | | | | |
| Example 3 | GH-3-1 | | | 3.71 | 119.80 | 0.23, 0.72 | 28.75 | 236 |
| | Compound | Compound | 40:60 | | | | | |
| | A22 | 78 | | | | | | |
| Example 4 | GH-4-1 | | | 3.72 | 115.00 | 0.23, 0.72 | 27.60 | 245 |
| | Compound | Compound | 40:60 | | | | | |
| | A31 | 79 | | | | | | |
| Example 5 | GH-5-1 | | | 3.71 | 119.50 | 0.23, 0.72 | 28.78 | 249 |
| | Compound | Compound | 40:60 | | | | | |
| | A41 | 80 | | | | | | |
| Example 6 | GH-6-1 | | | 3.76 | 115.10 | 0.23, 0.72 | 27.61 | 233 |
| | Compound | Compound | 40:60 | | | | | |
| | A55 | 76 | | | | | | |
| Example 7 | GH-7-1 | | | 3.75 | 115.80 | 0.23, 0.72 | 27.79 | 245 |
| | Compound | Compound | 40:60 | | | | | |
| | A62 | 77 | | | | | | |
| Example 8 | GH-8-1 | | | 3.73 | 117.00 | 0.23, 0.72 | 28.10 | 247 |
| | Compound | Compound | 40:60 | | | | | |
| | A73 | 80 | | | | | | |
| Example 9 | GH-9-1 | | | 3.74 | 117.63 | 0.23, 0.72 | 28.21 | 240 |
| | Compound | Compound | 40:60 | | | | | |
| | B4 | 79 | | | | | | |
| Example 10 | GH-10-1 | | | 3.72 | 115.43 | 0.23, 0.72 | 27.70 | 236 |
| | Compound | Compound | 40:60 | | | | | |
| | B19 | 75 | | | | | | |
| Example 11 | GH-11-1 | | | 3.78 | 115.08 | 0.23, 0.72 | 27.62 | 247 |
| | Compound | Compound | 40:60 | | | | | |
| | B29 | 77 | | | | | | |
| Example 12 | GH-12-1 | | | 3.74 | 118.80 | 0.23, 0.72 | 28.51 | 244 |
| | Compound | Compound | 40:60 | | | | | |
| | B36 | 78 | | | | | | |
| Example 13 | GH-13-1 | | | 3.77 | 116.20 | 0.23, 0.72 | 27.89 | 249 |
| | Compound | Compound | 40:60 | | | | | |
| | B44 | 80 | | | | | | |
| Example 14 | GH-14-1 | | | 3.77 | 118.70 | 0.23, 0.72 | 28.48 | 239 |
| | Compound | Compound | 40:60 | | | | | |
| | B58 | 79 | | | | | | |
| Example 15 | GH-15-1 | | | 3.73 | 116.50 | 0.23, 0.72 | 27.96 | 235 |
| | Compound | Compound | 40:60 | | | | | |
| | B68 | 76 | | | | | | |
| Example 16 | GH-16-1 | | | 3.71 | 118.66 | 0.23, 0.72 | 28.47 | 248 |
| | Compound | Compound | 40:60 | | | | | |
| | B74 | 77 | | | | | | |
| Example 17 | GH-17-1 | | | 3.78 | 116.38 | 0.23, 0.72 | 27.93 | 235 |
| | Compound | Compound | 40:60 | | | | | |
| | B84 | 76 | | | | | | |
| Example 18 | GH-18-1 | | | 3.77 | 117.30 | 0.23, 0.72 | 28.15 | 249 |
| | Compound | Compound | 40:60 | | | | | |
| | B95 | 80 | | | | | | |
| Example 19 | GH-19-1 | | | 3.78 | 118.73 | 0.23, 0.72 | 28.52 | 236 |
| | Compound | Compound | 40:60 | | | | | |
| | B104 | 79 | | | | | | |
| Example 20 | GH-20-1 | | | 3.78 | 116.42 | 0.23, 0.72 | 27.94 | 235 |
| | Compound | Compound | 40:60 | | | | | |
| | B116 | 77 | | | | | | |
| Example 21 | GH-21-1 | | | 3.77 | 116.05 | 0.23, 0.72 | 27.81 | 238 |
| | Compound | Compound | 40:60 | | | | | |
| | B128 | 78 | | | | | | |
| Example 22 | GH-22-1 | | | 3.71 | 115.50 | 0.23, 0.72 | 27.72 | 239 |
| | Compound | Compound | 40:60 | | | | | |
| | B133 | 79 | | | | | | |
| Example 23 | GH-23-1 | | | 3.77 | 117.93 | 0.23, 0.72 | 28.31 | 235 |
| | Compound | Compound | 40:60 | | | | | |
| | B143 | 78 | | | | | | |
| Example 24 | GH-24-1 | | | 3.76 | 116.18 | 0.23, 0.72 | 27.88 | 216 |
| | Compound | Compound | 40:60 | | | | | |
| | C4 | 76 | | | | | | |
| Example 25 | GH-25-1 | | | 3.78 | 117.88 | 0.23, 0.72 | 28.29 | 211 |
| | Compound | Compound | 40:60 | | | | | |
| | C11 | 78 | | | | | | |
| Example 26 | GH-26-1 | | | 3.78 | 119.00 | 0.23, 0.72 | 28.56 | 205 |
| | Compound | Compound | 40:60 | | | | | |
| | C14 | 80 | | | | | | |
| Example 27 | GH-27-1 | | | 3.73 | 117.55 | 0.23, 0.72 | 28.18 | 220 |
| | Compound | Compound | 40:60 | | | | | |
| | C25 | 77 | | | | | | |
| Example 28 | GH-28-1 | | | 3.74 | 116.00 | 0.23, 0.72 | 27.80 | 218 |
| | Compound | Compound | 40:60 | | | | | |
| | C33 | 76 | | | | | | |
| Example 29 | GH-29-1 | | | 3.74 | 115.20 | 0.23, 0.72 | 27.64 | 219 |
| | Compound | Compound | 40:60 | | | | | |
| | C42 | 80 | | | | | | |
| Example 30 | GH-30-1 | | | 3.74 | 118.07 | 0.23, 0.72 | 28.33 | 215 |
| | Compound | Compound | 40:60 | | | | | |
| | C52 | 80 | | | | | | |
| Example 31 | GH-31-1 | | | 3.75 | 119.20 | 0.23, 0.72 | 28.61 | 206 |
| | Compound | Compound | 40:60 | | | | | |
| | C60 | 79 | | | | | | |
| Example 32 | GH-32-1 | | | 3.73 | 115.46 | 0.23, 0.72 | 27.71 | 210 |
| | Compound | Compound | 40:60 | | | | | |
| | D3 | 78 | | | | | | |
| Example 33 | GH-33-1 | | | 3.74 | 115.60 | 0.23, 0.72 | 27.74 | 220 |
| | Compound | Compound | 40:60 | | | | | |
| | D10 | 77 | | | | | | |
| Example 34 | GH-34-1 | | | 3.76 | 118.71 | 0.23, 0.72 | 28.49 | 219 |
| | Compound | Compound | 40:60 | | | | | |
| | D26 | 76 | | | | | | |
| Example 35 | GH-35-1 | | | 3.77 | 116.60 | 0.23, 0.72 | 28.00 | 210 |
| | Compound | Compound | 40:60 | | | | | |
| | D33 | 78 | | | | | | |
| Example 36 | GH-36-1 | | | 3.72 | 115.30 | 0.23, 0.72 | 27.67 | 206 |
| | Compound | Compound | 40:60 | | | | | |
| | D51 | 79 | | | | | | |
| Example 37 | GH-37-1 | | | 3.77 | 117.60 | 0.23, 0.72 | 28.22 | 218 |
| | Compound | Compound | 40:60 | | | | | |
| | D61 | 80 | | | | | | |
| Example 38 | GH-38-1 | | | 3.74 | 116.13 | 0.23, 0.72 | 27.83 | 214 |
| | Compound | Compound | 40:60 | | | | | |
| | C25 | 77 | | | | | | |
| Example 39 | GH-39-1 | | | 3.79 | 115.02 | 0.23, 0.72 | 27.57 | 210 |
| | Compound | Compound | 50:50 | | | | | |
| | C25 | 77 | | | | | | |
| Comparative | GH-D1-1 | | | 3.75 | 84.20 | 0.23, 0.72 | 20.20 | 120 |
| Example 1 | Compound | Compound | 40:60 | | | | | |
| | I | 76 | | | | | | |
| Comparative | GH-D2-1 | | | 3.79 | 89.80 | 0.23, 0.72 | 21.55 | 137 |
| Example 2 | Compound | Compound | 40:60 | | | | | |
| | I | 77 | | | | | | |
| Comparative | GH-D3-1 | | | 3.80 | 100.80 | 0.23, 0.72 | 24.19 | 175 |
| Example 3 | Compound | Compound | 40:60 | | | | | |
| | II | 80 | | | | | | |
| Comparative | GH-D4-1 | | | 3.80 | 100.56 | 0.23, 0.72 | 24.13 | 160 |
| Example 4 | Compound | Compound | 40:60 | | | | | |
| | II | 77 | | | | | | |
| Comparative | GH-D5-1 | | | 3.77 | 103.10 | 0.23, 0.72 | 24.74 | 147 |
| Example 5 | Compound | Compound | 40:60 | | | | | |
| | III | 79 | | | | | | |
| Comparative | GH-D6-1 | | | 3.78 | 102.90 | 0.23, 0.72 | 24.69 | 159 |
| Example 6 | Compound | Compound | 40:60 | | | | | |
| | III | 77 | | | | | | |

It can be seen from the above table that, compared to Comparative Examples 1 to 6, in Examples 1 to 39, the current efficiency is increased by 11.5% at least, and the T95 lifetime is prolonged by 17.1% at least.

Compared to Comparative Examples 1 to 2, when the compound of Formula 1 of the present disclosure is used as a green electron type host material, the T95 lifetime of the prepared device is prolonged remarkably compared to a device prepared from the compound I. The reason may lie in that, compared to the compound I, in the organic compound of the present disclosure, specific sites of a core carbazole structure are deuterated, and thus its photoelectric stability is improved.

Compared to Comparative Examples 3 to 6, when the compound of Formula 1 of the present disclosure is used as a green electron type host material, the prepared devices have remarkably improved current efficiency and T95 lifetimes compared to devices prepared from the compounds II and III. The reason may lie in that, compared to the compounds II and III, in the organic compound of the present disclosure, biphenyl/triphenyl groups with relatively large steric hindrance are introduced to carbazole groups, inter-molecular interactions are further lowered, molecule stacking is relieved, the amorphous stability of the material is improved, and thus the film forming property of the material is better.

Other embodiments of the present disclosure will be apparent to those skilled in the art from consideration of the specification and practice of the invention disclosed here. The present disclosure is intended to cover any variations, uses, or adaptations of the present disclosure following the general principles of the present disclosure and including such departures from the present disclosure as come within known or customary practice in the art.

1. An organic compound, wherein the organic compound has a structure as shown in a Formula 1: 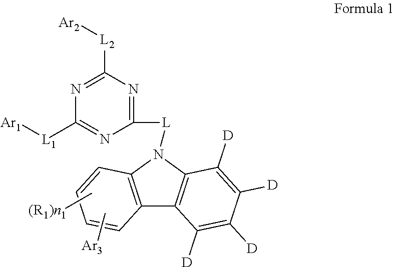
wherein, Ar₁ and Ar₂ are the same or different, and are respectively and independently selected from a substituted or unsubstituted aryl with 6 to 30 carbon atoms, a substituted or unsubstituted dibenzofuranyl, and a substituted or unsubstituted dibenzothienyl;
L, L₁ and L₂ are the same or different, and are respectively and independently selected from a single bond, and a substituted or unsubstituted arylene with 6 to 30 carbon atoms;
Ar₃ is selected from a substituted or unsubstituted biphenyl;
substituent(s) in Ar₃ are each independently selected from a deuterium, a pentadeuterophenyl and a phenyl;
R₁ is a hydrogen or a deuterium;
n₁ is the number of R₁ and selected from 1, 2 or 3; and
substituent(s) in L, L₁, L₂, Ar₁ and Ar₂ are the same or different, and are respectively and independently selected from a deuterium, a cyano, a halogen group, an alkyl with 1 to 10 carbon atoms, a haloalkyl with 1 to 10 carbon atoms, a deuteroalkyl with 1 to 10 carbon atoms, an aryl with 6 to 20 carbon atoms, a deuteroaryl with 6 to 20 carbon atoms, a haloaryl with 6 to 20 carbon atoms, a heteroaryl with 5 to 20 carbon atoms, and a cycloalkyl with 3 to 10 carbon atoms.

2. The organic compound according to claim 1, wherein Ar₃ is selected from a group consisting of the following groups:

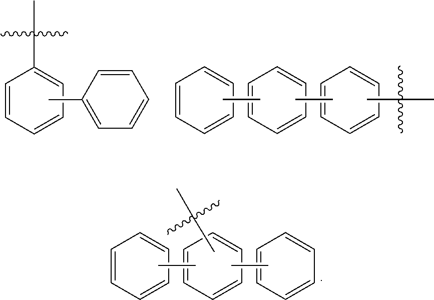

3. The organic compound according to claim 1, wherein L, L₁ and L₂ are the same or different, and are respectively and independently selected from a single bond, and a substituted or unsubstituted arylene with 6 to 12 carbon atoms; and
optionally, substituent(s) in L, L₁ and L₂ are the same or different, and are respectively and independently selected from a deuterium, a halogen group, a cyano, an alkyl with 1 to 5 carbon atoms, and a phenyl.

4. The organic compound according to claim 1, wherein L, L₁ and L₂ are the same or different, and are respectively and independently selected from a single bond, a substituted or unsubstituted phenylene, a substituted or unsubstituted naphthylene and a substituted or unsubstituted biphenylene; and
optionally, substituent(s) in L, L₁ and L₂ are the same or different, and are respectively and independently selected from a deuterium, a fluorine, a cyano, a methyl, an ethyl, a n-propyl, an isopropyl, a tert-butyl, and a phenyl.

5. The organic compound according to claim 1, wherein Ar₁ and Ar₂ are the same or different, and are respectively and independently selected from a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted terphenyl, a substituted or unsubstituted dibenzofuranyl, and a substituted or unsubstituted dibenzothienyl; and
optionally, substituent(s) in Ar₁ and Ar₂ are the same or different and each independently selected from a deuterium, a fluorine, a cyano, a methyl, an ethyl, a n-propyl, an isopropyl, a tert-butyl, a phenyl, and a pentadeuterophenyl.

6. The organic compound according to claim 1, wherein 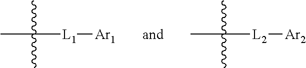
are the same or different and each independently selected from a group consisting of the following groups:

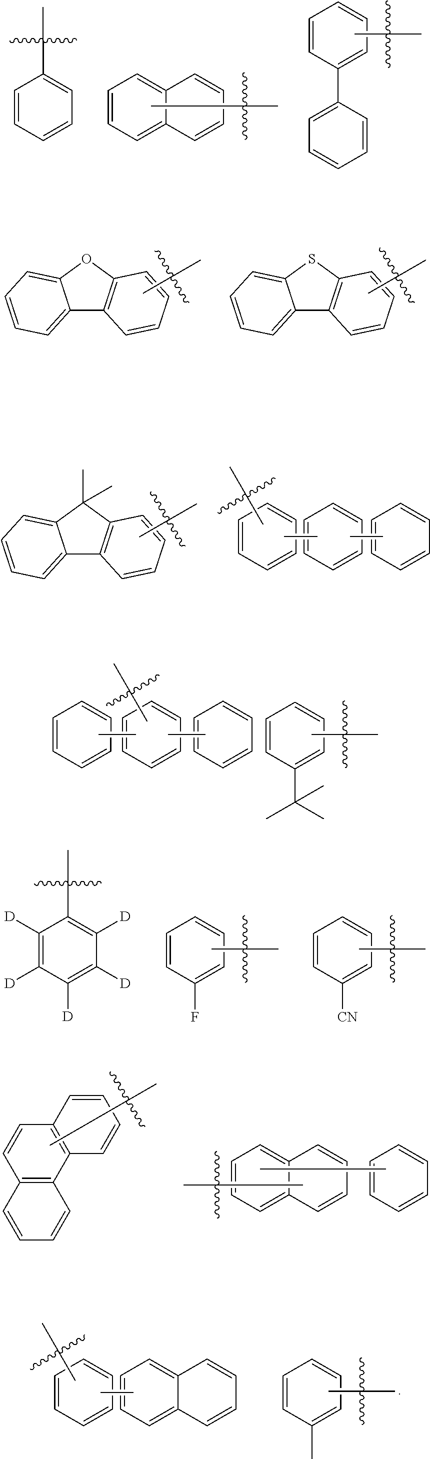

7. The organic compound according to claim 1, wherein 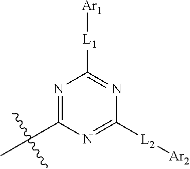
in Formula 1 is selected from a group consisting of the following groups:

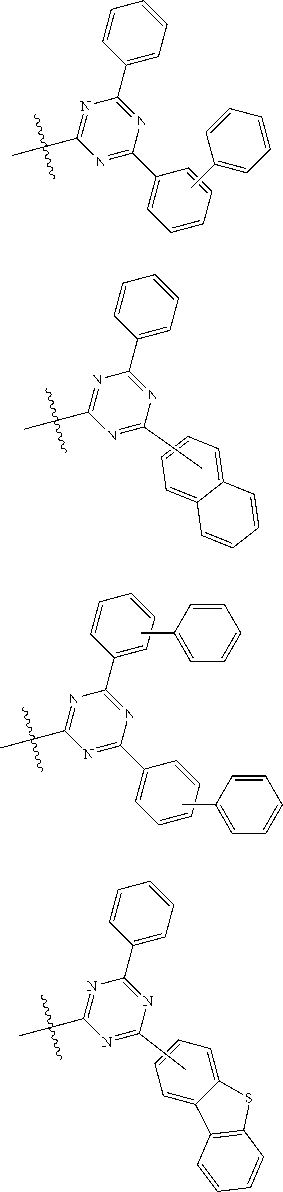

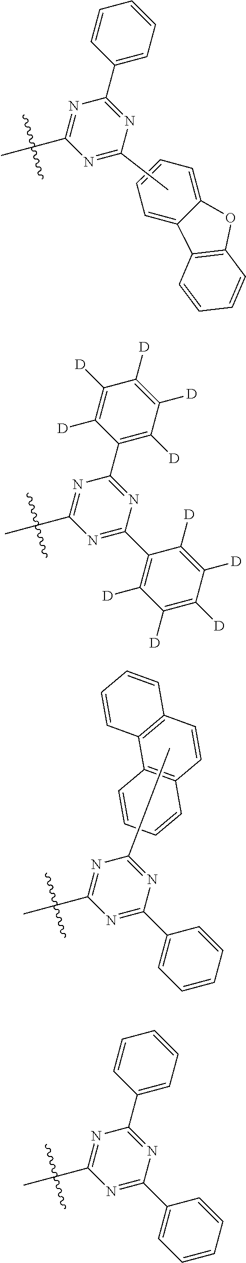

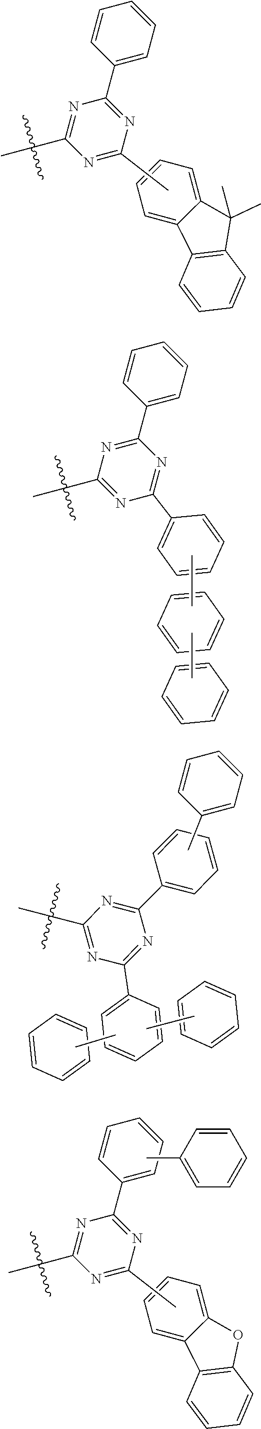

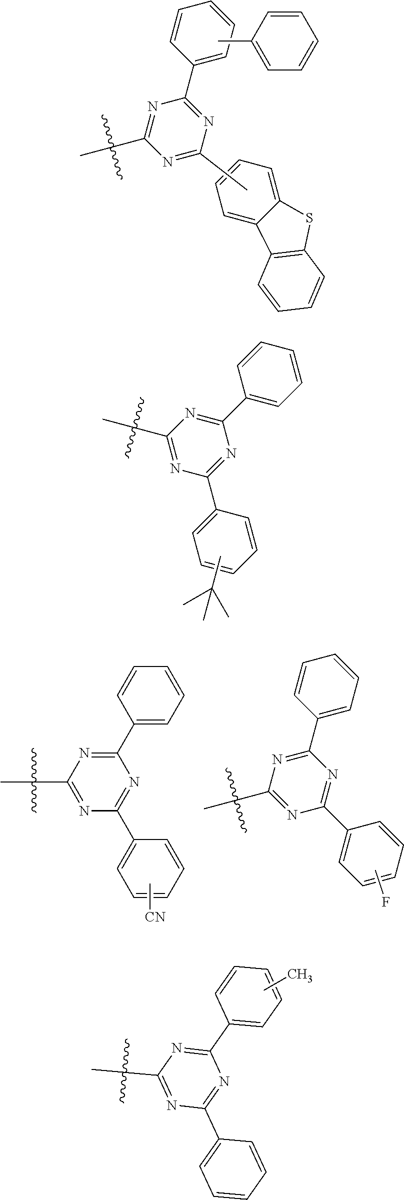

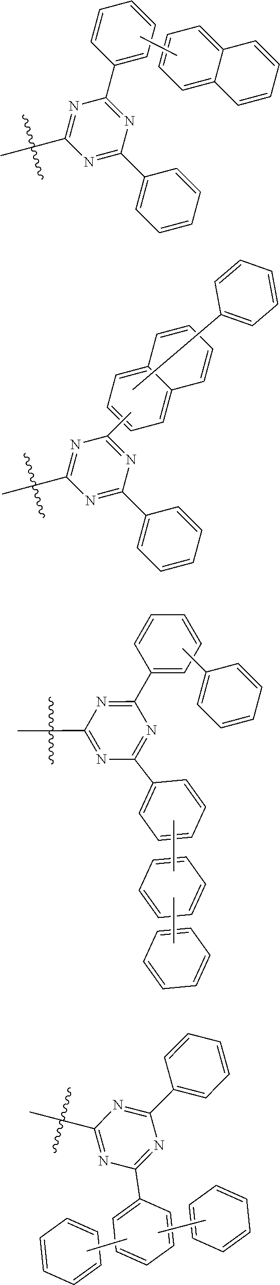

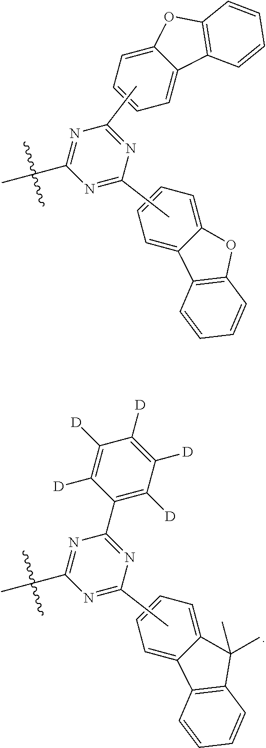

8. The organic compound according to claim 1, wherein the organic compound is selected from a group consisting of the following compounds:

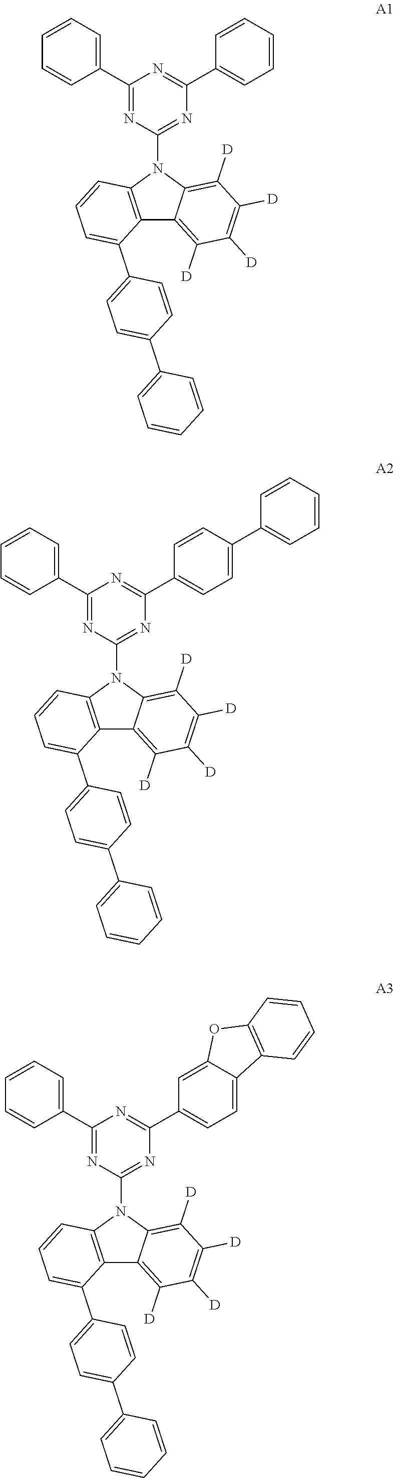 [SMILES: [2H]c1c([2H])c([2H])c2c(c1[2H])c1c(-c3ccc(-c4ccccc4)cc3)cccc1n2-c1nc(-c2ccccc2)nc(-c2ccccc2)n1 ; [2H]c1c([2H])c([2H])c2c(c1[2H])c1c(-c3ccc(-c4ccccc4)cc3)cccc1n2-c1nc(-c2ccccc2)nc(-c2ccc(-c3ccccc3)cc2)n1 ; [2H]c1c([2H])c([2H])c2c(c1[2H])c1c(-c3ccc(-c4ccccc4)cc3)cccc1n2-c1nc(-c2ccccc2)nc(-c2ccc3c(c2)oc2ccccc23)n1]

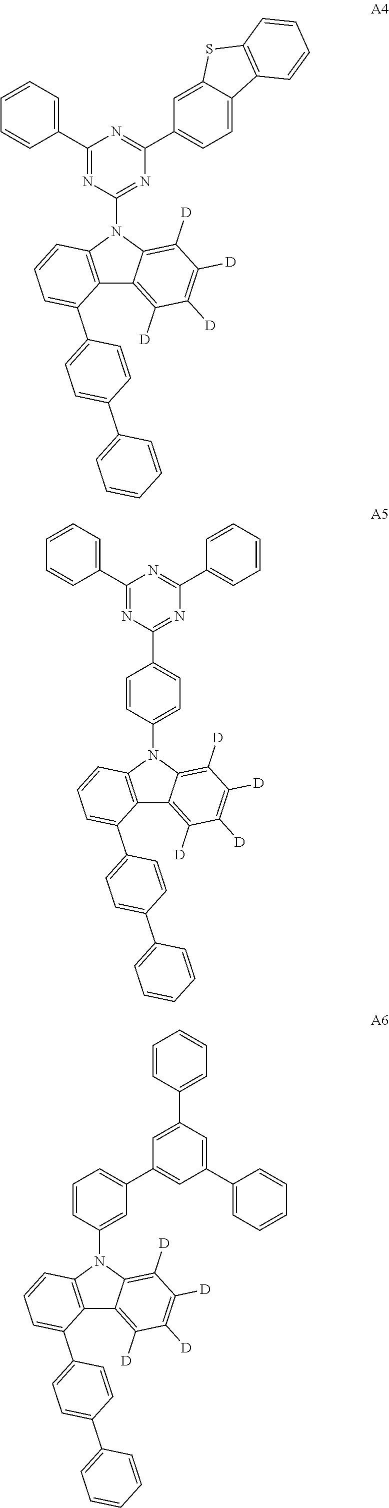

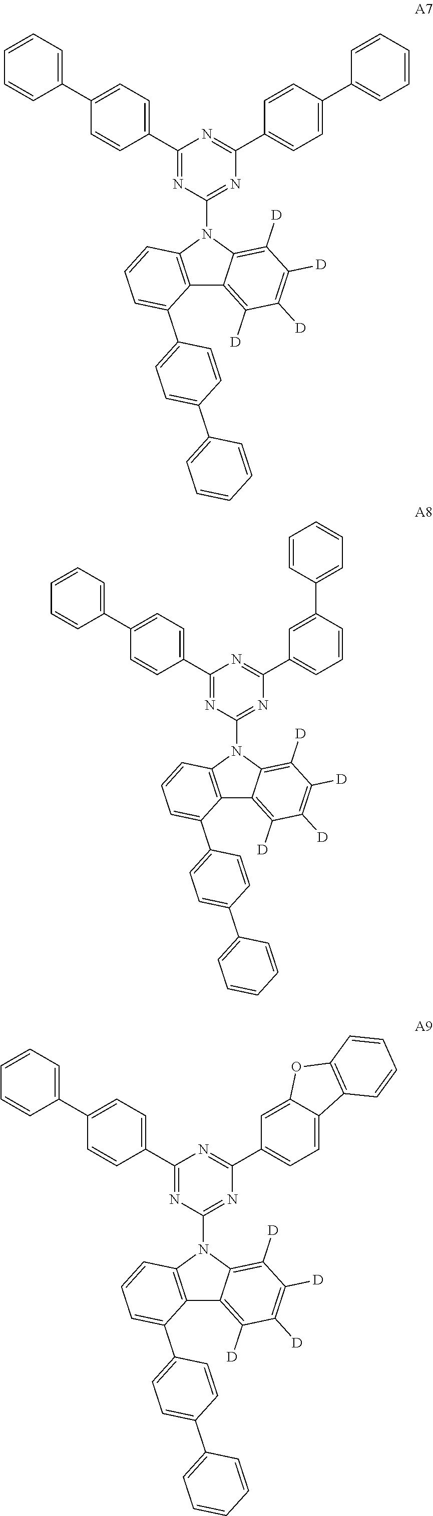 [SMILES: [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc(-c3ccccc3)cc2)n1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2cccc(-c3ccccc3)c2)n1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3c(c2)oc2ccccc23)n1]

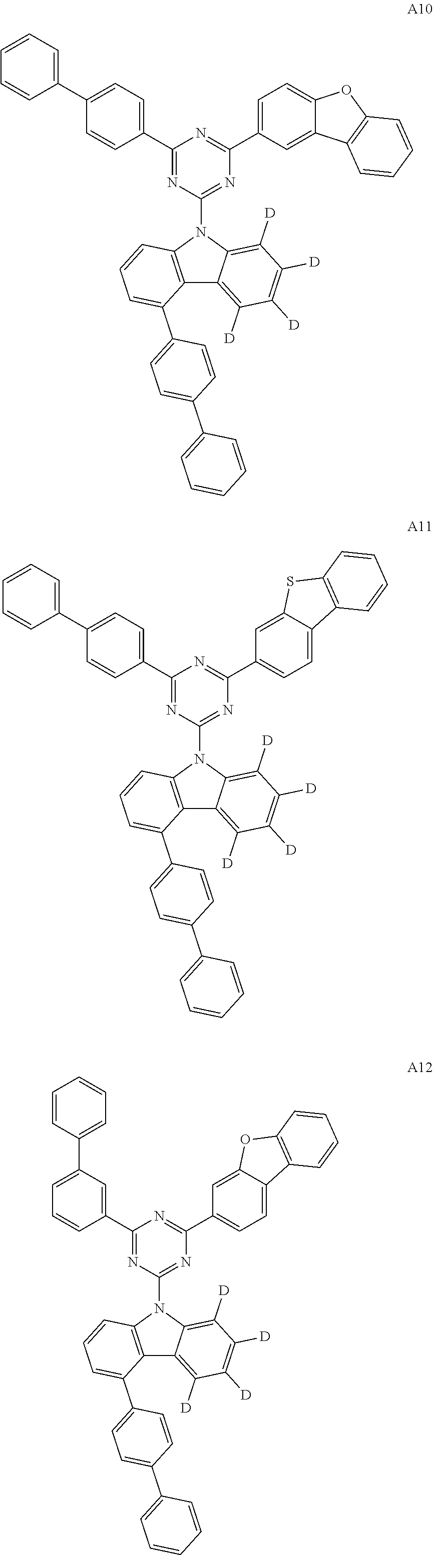 [SMILES: [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3oc4ccccc4c3c2)n1 ; [2H]c1c([2H])c([2H])c2c(c1[2H])c1c(-c3ccc(-c4ccccc4)cc3)cccc1n2-c1nc(-c2ccc(-c3ccccc3)cc2)nc(-c2ccc3c(c2)sc2ccccc23)n1 ; [2H]c1c([2H])c([2H])c2c(c1[2H])c1c(-c3ccc(-c4ccccc4)cc3)cccc1n2-c1nc(-c2cccc(-c3ccccc3)c2)nc(-c2ccc3c(c2)oc2ccccc23)n1]

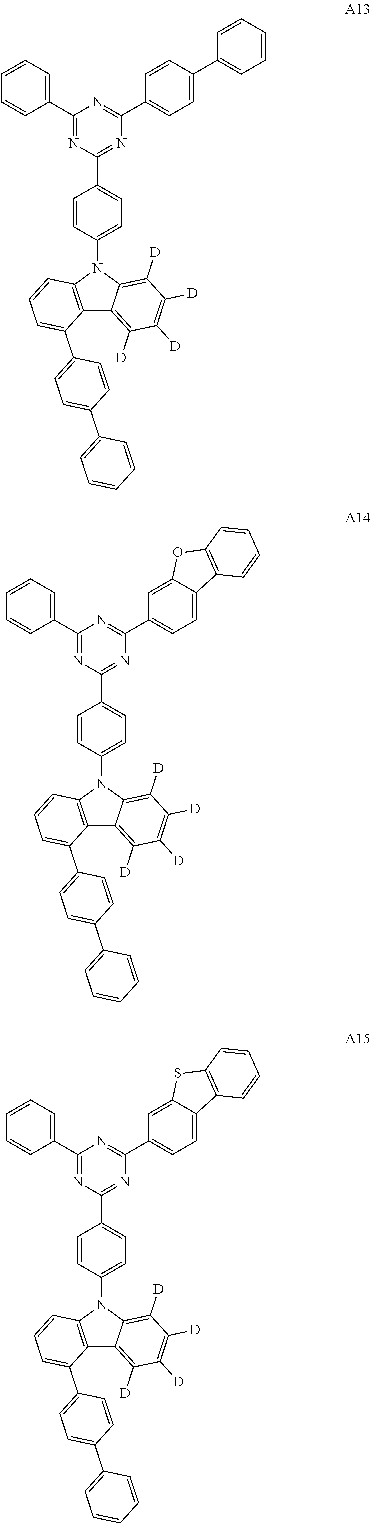 [SMILES: [2H]c1c([2H])c([2H])c2c(c1[2H])c1c(-c3ccc(-c4ccccc4)cc3)cccc1n2-c1ccc(-c2nc(-c3ccccc3)nc(-c3ccc(-c4ccccc4)cc3)n2)cc1 ; [2H]c1c([2H])c([2H])c2c(c1[2H])c1c(-c3ccc(-c4ccccc4)cc3)cccc1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3ccc(-c4ccccc4)cc3)cccc1n2-c1ccc(-c2nc(-c3ccccc3)nc(-c3ccc4c(c3)sc3ccccc34)n2)cc1]

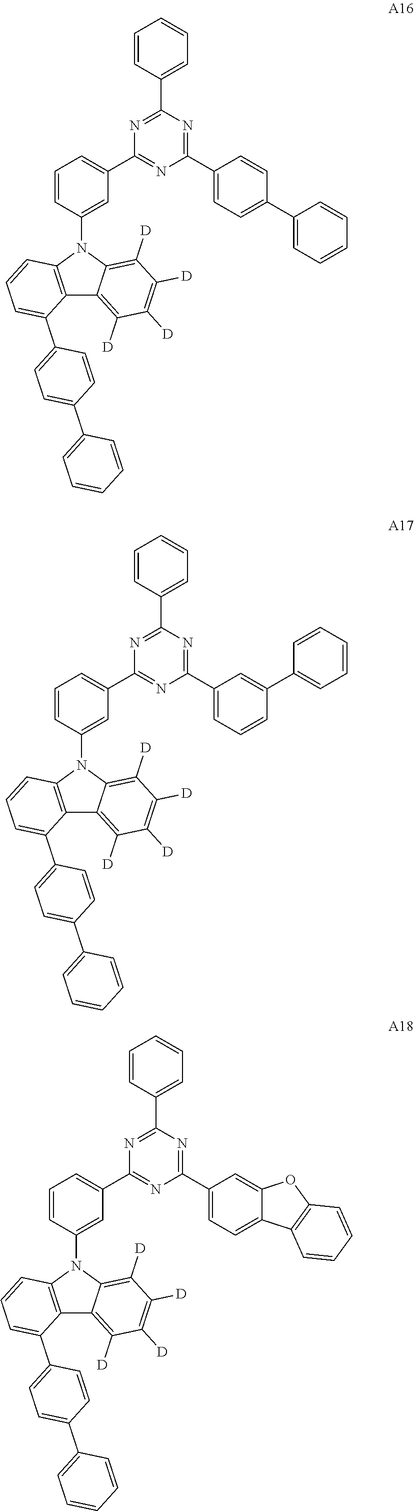 [SMILES: [2H]c1c([2H])c([2H])c2c(c1[2H])c1c(-c3ccc(-c4ccccc4)cc3)cccc1n2-c1cccc(-c2nc(-c3ccccc3)nc(-c3ccc(-c4ccccc4)cc3)n2)c1 ; [2H]c1c([2H])c([2H])c2c(c1[2H])c1c(-c3ccc(-c4ccccc4)cc3)cccc1n2-c1cccc(-c2nc(-c3ccccc3)nc(-c3cccc(-c4ccccc4)c3)n2)c1 ; [2H]c1c([2H])c([2H])c2c(c1[2H])c1c(-c3ccc(-c4ccccc4)cc3)cccc1n2-c1cccc(-c2nc(-c3ccccc3)nc(-c3ccc4c(c3)oc3ccccc34)n2)c1]

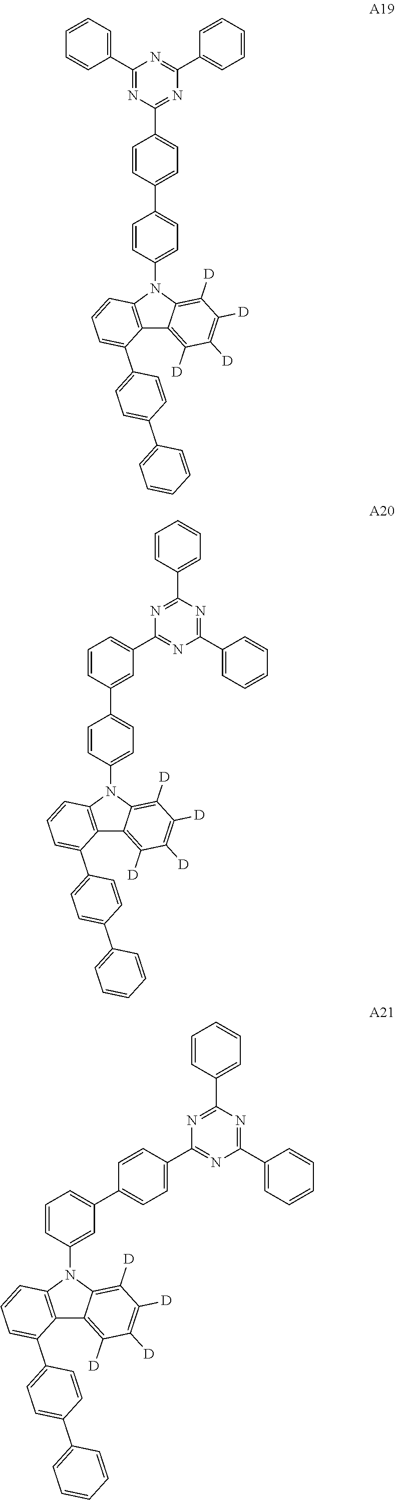

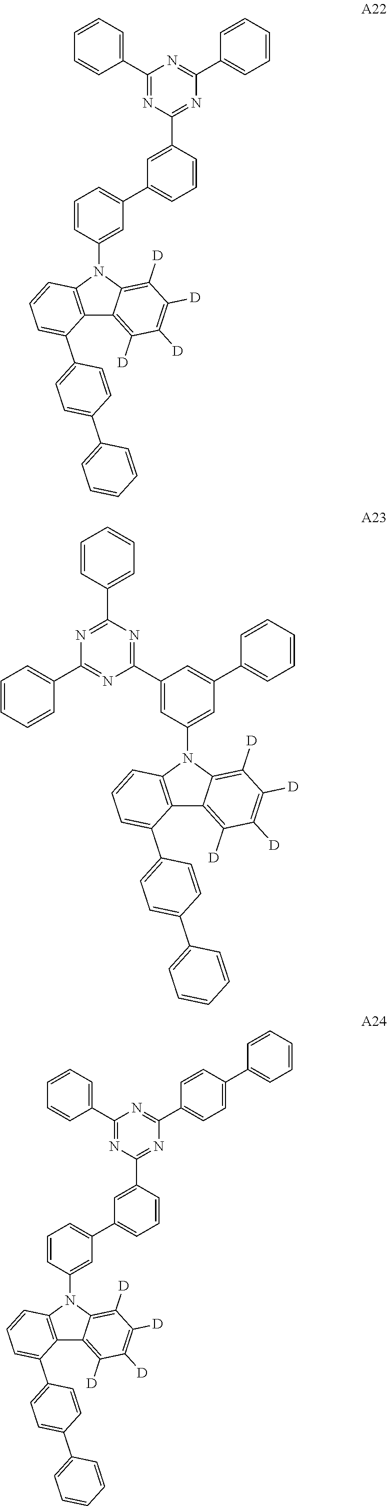

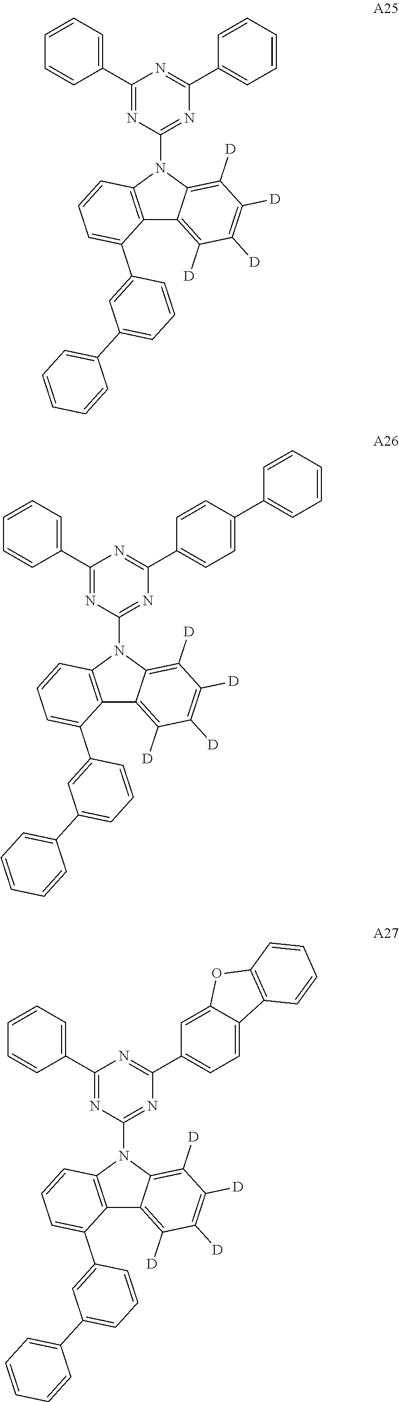 [SMILES: [2H]c1c([2H])c([2H])c2c(c1[2H])c1c(-c3cccc(-c4ccccc4)c3)cccc1n2-c1nc(-c2ccccc2)nc(-c2ccccc2)n1 ; [2H]c1c([2H])c([2H])c2c(c1[2H])c1c(-c3cccc(-c4ccccc4)c3)cccc1n2-c1nc(-c2ccccc2)nc(-c2ccc(-c3ccccc3)cc2)n1 ; [2H]c1c([2H])c([2H])c2c(c1[2H])c1c(-c3cccc(-c4ccccc4)c3)cccc1n2-c1nc(-c2ccccc2)nc(-c2ccc3c(c2)oc2ccccc23)n1]

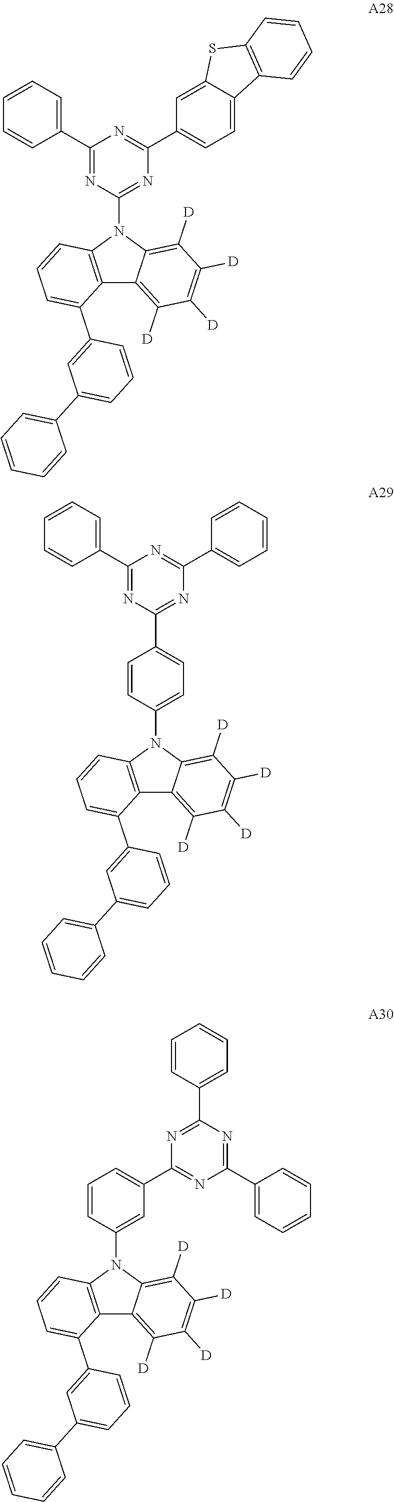

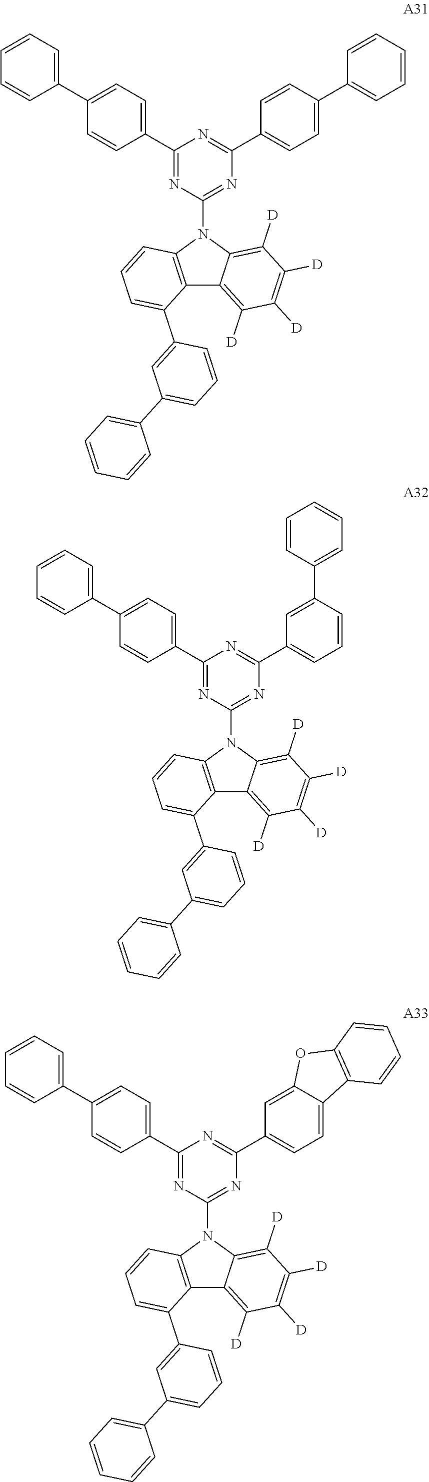 [SMILES: [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc(-c3ccccc3)cc2)n1 ; [2H]c1c([2H])c([2H])c2c(c1[2H])c1c(-c3cccc(-c4ccccc4)c3)cccc1n2-c1nc(-c2ccc(-c3ccccc3)cc2)nc(-c2cccc(-c3ccccc3)c2)n1 ; [2H]c1c([2H])c([2H])c2c(c1[2H])c1c(-c3cccc(-c4ccccc4)c3)cccc1n2-c1nc(-c2ccc(-c3ccccc3)cc2)nc(-c2ccc3c(c2)oc2ccccc23)n1]

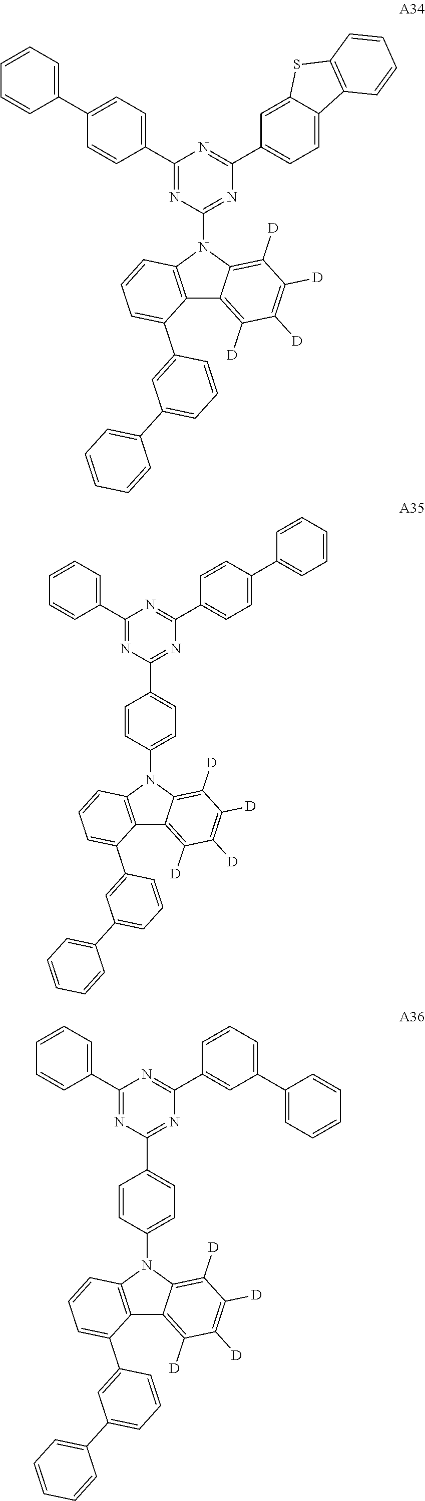

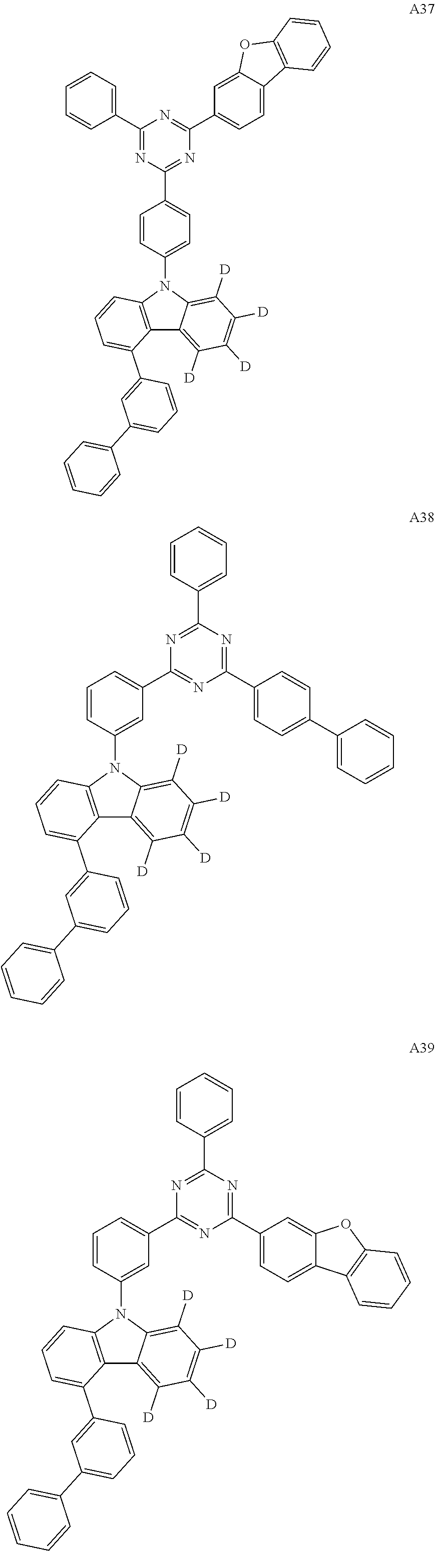 [SMILES: [2H]c1c([2H])c([2H])c2c(c1[2H])c1c(-c3cccc(-c4ccccc4)c3)cccc1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3cccc(-c4ccccc4)c3)cccc1n2-c1cccc(-c2nc(-c3ccccc3)nc(-c3ccc(-c4ccccc4)cc3)n2)c1 ; [2H]c1c([2H])c([2H])c2c(c1[2H])c1c(-c3cccc(-c4ccccc4)c3)cccc1n2-c1cccc(-c2nc(-c3ccccc3)nc(-c3ccc4c(c3)oc3ccccc34)n2)c1]

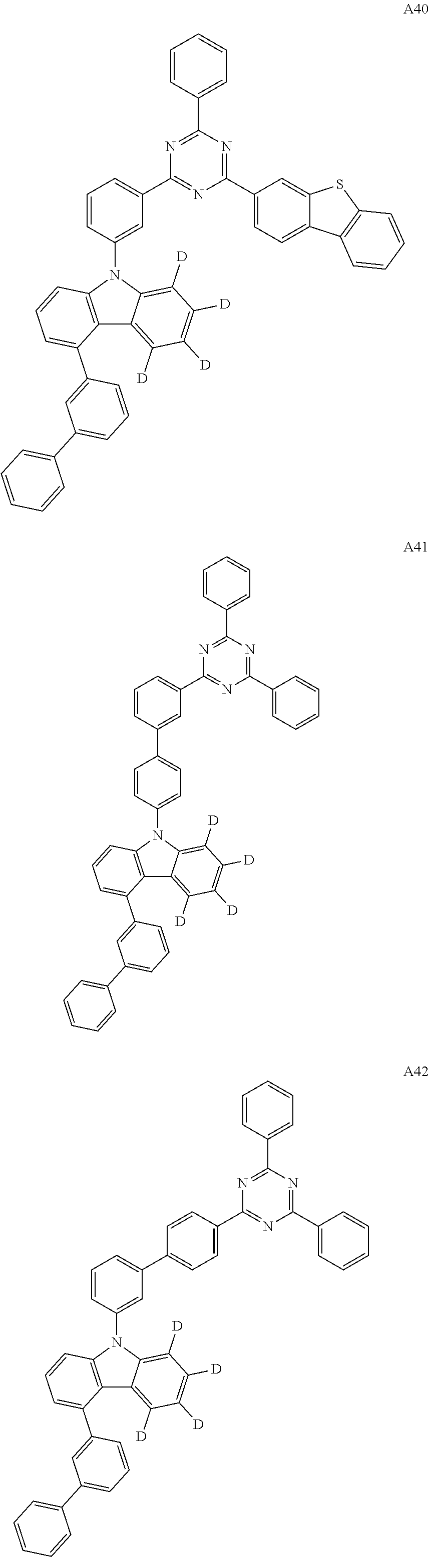 [SMILES: [2H]c1c([2H])c([2H])c2c(c1[2H])c1c(-c3cccc(-c4ccccc4)c3)cccc1n2-c1cccc(-c2nc(-c3ccccc3)nc(-c3ccc4c(c3)sc3ccccc34)n2)c1 ; [2H]c1c([2H])c([2H])c2c(c1[2H])c1c(-c3cccc(-c4ccccc4)c3)cccc1n2-c1ccc(-c2cccc(-c3nc(-c4ccccc4)nc(-c4ccccc4)n3)c2)cc1 ; [2H]c1c([2H])c([2H])c2c(c1[2H])c1c(-c3cccc(-c4ccccc4)c3)cccc1n2-c1cccc(-c2ccc(-c3nc(-c4ccccc4)nc(-c4ccccc4)n3)cc2)c1]

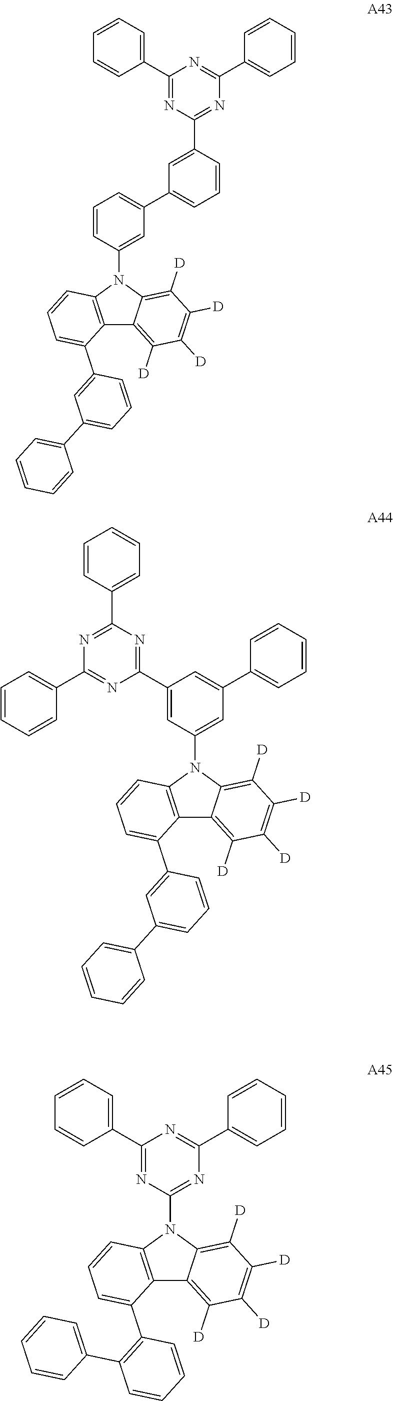 [SMILES: [2H]c1c([2H])c([2H])c2c(c1[2H])c1c(-c3cccc(-c4ccccc4)c3)cccc1n2-c1cccc(-c2cccc(-c3nc(-c4ccccc4)nc(-c4ccccc4)n3)c2)c1 ; [2H]c1c([2H])c([2H])c2c(c1[2H])c1c(-c3cccc(-c4ccccc4)c3)cccc1n2-c1cc(-c2ccccc2)cc(-c2nc(-c3ccccc3)nc(-c3ccccc3)n2)c1 ; [2H]c1c([2H])c([2H])c2c(c1[2H])c1c(-c3ccccc3-c3ccccc3)cccc1n2-c1nc(-c2ccccc2)nc(-c2ccccc2)n1]

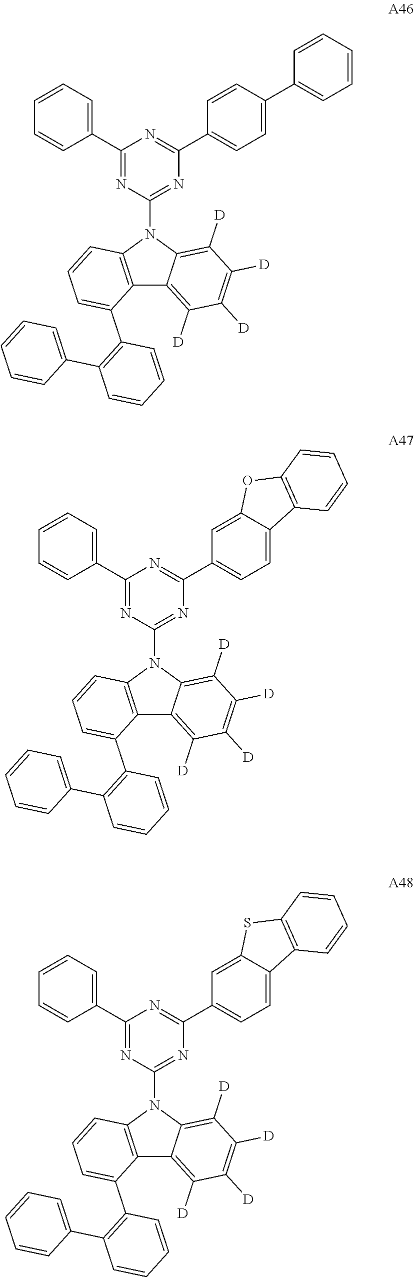 [SMILES: [2H]c1c([2H])c([2H])c2c(c1[2H])c1c(-c3ccccc3-c3ccccc3)cccc1n2-c1nc(-c2ccccc2)nc(-c2ccc(-c3ccccc3)cc2)n1 ; [2H]c1c([2H])c([2H])c2c(c1[2H])c1c(-c3ccccc3-c3ccccc3)cccc1n2-c1nc(-c2ccccc2)nc(-c2ccc3c(c2)oc2ccccc23)n1 ; [2H]c1c([2H])c([2H])c2c(c1[2H])c1c(-c3ccccc3-c3ccccc3)cccc1n2-c1nc(-c2ccccc2)nc(-c2ccc3c(c2)sc2ccccc23)n1]

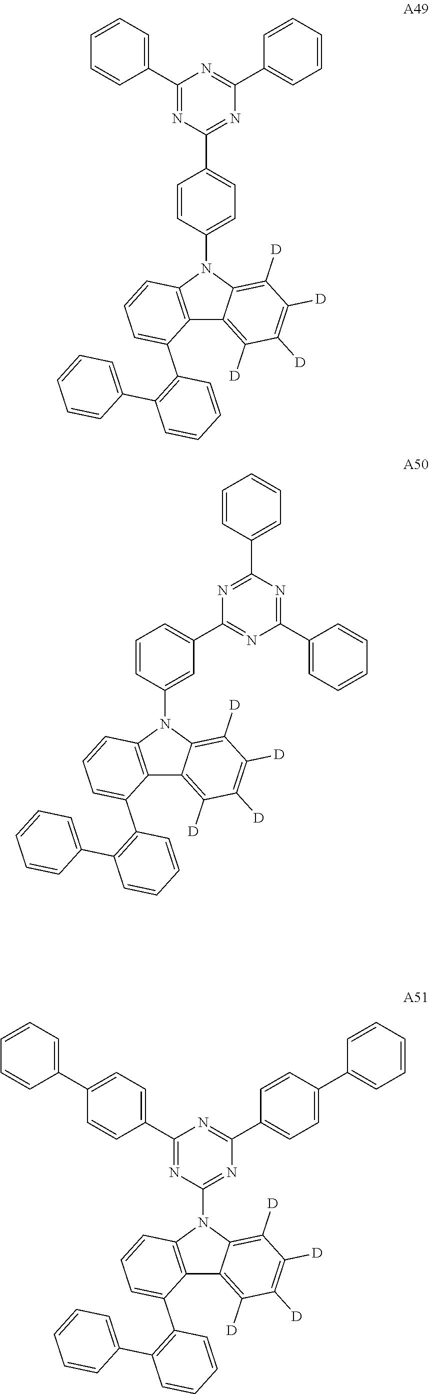 [SMILES: [2H]c1c([2H])c([2H])c2c(c1[2H])c1c(-c3ccccc3-c3ccccc3)cccc1n2-c1ccc(-c2nc(-c3ccccc3)nc(-c3ccccc3)n2)cc1 ; [2H]c1c([2H])c([2H])c2c(c1[2H])c1c(-c3ccccc3-c3ccccc3)cccc1n2-c1cccc(-c2nc(-c3ccccc3)nc(-c3ccccc3)n2)c1 ; [2H]c1c([2H])c([2H])c2c(c1[2H])c1c(-c3ccccc3-c3ccccc3)cccc1n2-c1nc(-c2ccc(-c3ccccc3)cc2)nc(-c2ccc(-c3ccccc3)cc2)n1]

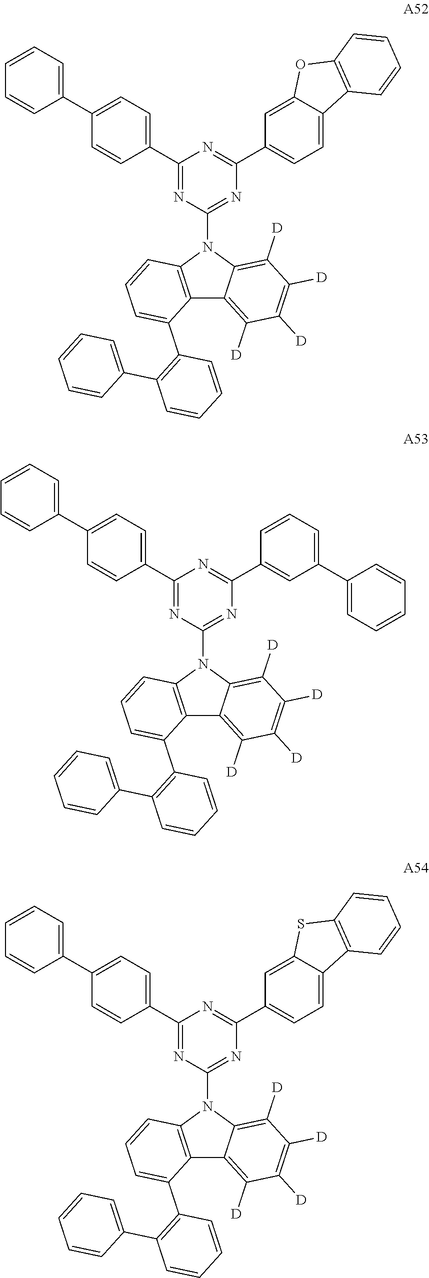 [SMILES: [2H]c1c([2H])c([2H])c2c(c1[2H])c1c(-c3ccccc3-c3ccccc3)cccc1n2-c1nc(-c2ccc(-c3ccccc3)cc2)nc(-c2ccc3c(c2)oc2ccccc23)n1 ; [2H]c1c([2H])c([2H])c2c(c1[2H])c1c(-c3ccccc3-c3ccccc3)cccc1n2-c1nc(-c2ccc(-c3ccccc3)cc2)nc(-c2cccc(-c3ccccc3)c2)n1 ; [2H]c1c([2H])c([2H])c2c(c1[2H])c1c(-c3ccccc3-c3ccccc3)cccc1n2-c1nc(-c2ccc(-c3ccccc3)cc2)nc(-c2ccc3c(c2)sc2ccccc23)n1]

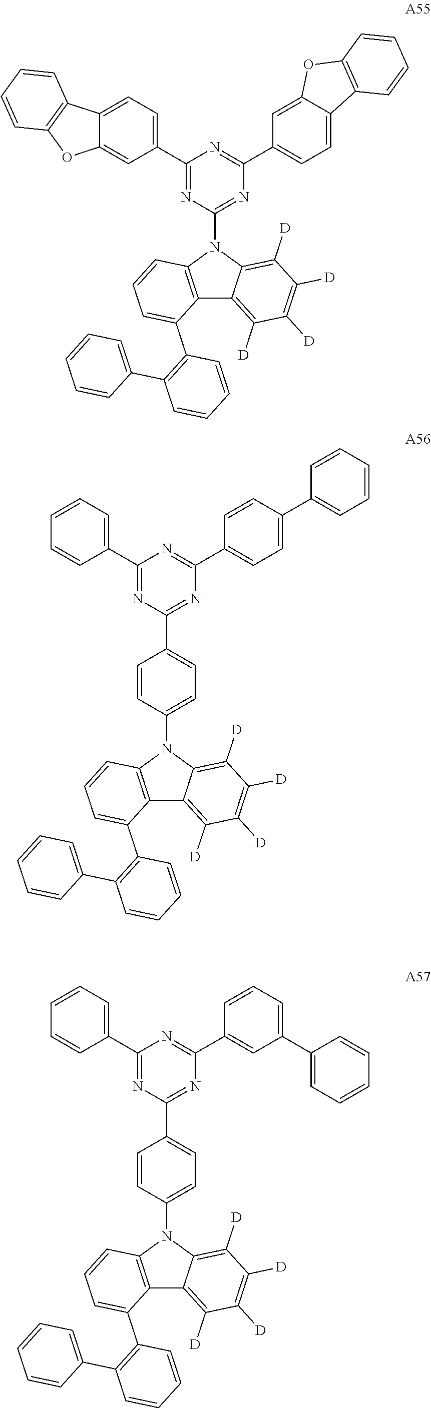 [SMILES: [2H]c1c([2H])c([2H])c2c(c1[2H])c1c(-c3ccccc3-c3ccccc3)cccc1n2-c1nc(-c2ccc3c(c2)oc2ccccc23)nc(-c2ccc3c(c2)oc2ccccc23)n1 ; [2H]c1c([2H])c([2H])c2c(c1[2H])c1c(-c3ccccc3-c3ccccc3)cccc1n2-c1ccc(-c2nc(-c3ccccc3)nc(-c3ccc(-c4ccccc4)cc3)n2)cc1 ; [2H]c1c([2H])c([2H])c2c(c1[2H])c1c(-c3ccccc3-c3ccccc3)cccc1n2-c1ccc(-c2nc(-c3ccccc3)nc(-c3cccc(-c4ccccc4)c3)n2)cc1]

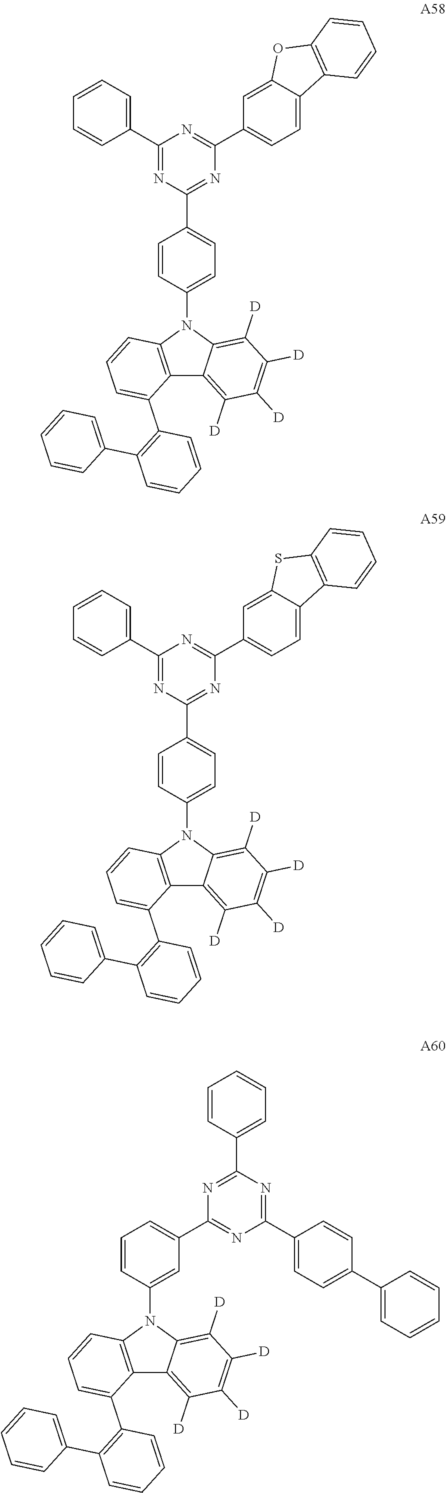 [SMILES: [2H]c1c([2H])c([2H])c2c(c1[2H])c1c(-c3ccccc3-c3ccccc3)cccc1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3ccccc3-c3ccccc3)cccc1n2-c1ccc(-c2nc(-c3ccccc3)nc(-c3ccc4c(c3)sc3ccccc34)n2)cc1 ; [2H]c1c([2H])c([2H])c2c(c1[2H])c1c(-c3ccccc3-c3ccccc3)cccc1n2-c1cccc(-c2nc(-c3ccccc3)nc(-c3ccc(-c4ccccc4)cc3)n2)c1]

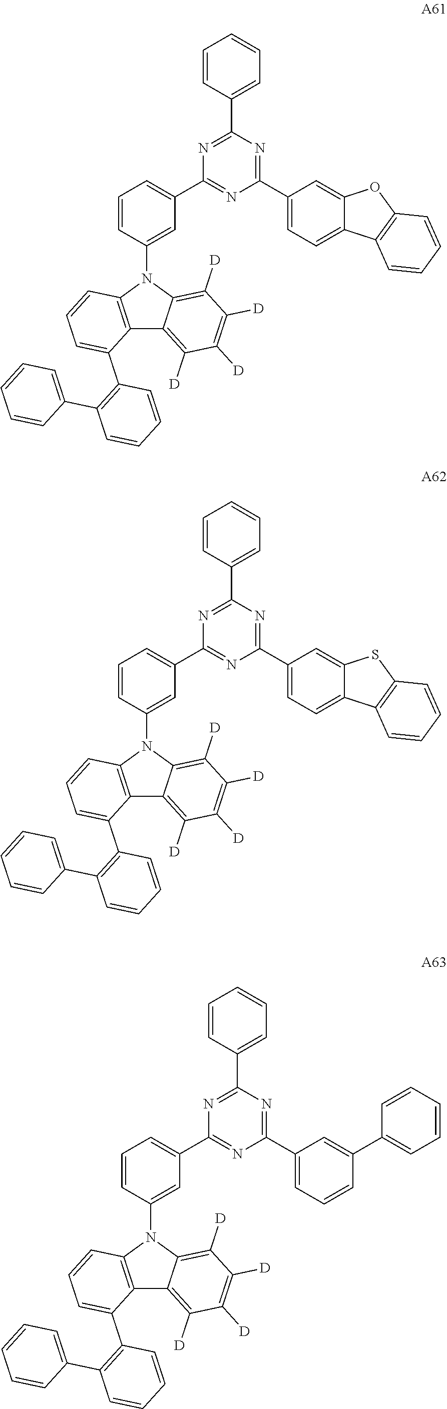 [SMILES: [2H]c1c([2H])c([2H])c2c(c1[2H])c1c(-c3ccccc3-c3ccccc3)cccc1n2-c1cccc(-c2nc(-c3ccccc3)nc(-c3ccc4c(c3)oc3ccccc34)n2)c1 ; [2H]c1c([2H])c([2H])c2c(c1[2H])c1c(-c3ccccc3-c3ccccc3)cccc1n2-c1cccc(-c2nc(-c3ccccc3)nc(-c3ccc4c(c3)sc3ccccc34)n2)c1 ; [2H]c1c([2H])c([2H])c2c(c1[2H])c1c(-c3ccccc3-c3ccccc3)cccc1n2-c1cccc(-c2nc(-c3ccccc3)nc(-c3cccc(-c4ccccc4)c3)n2)c1]

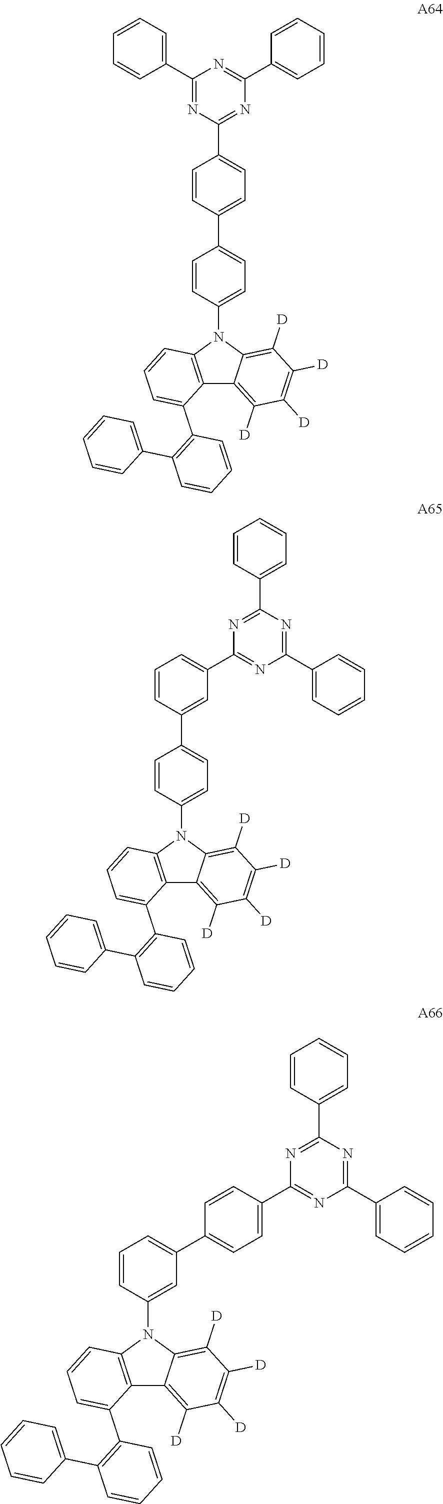 [SMILES: [2H]c1c([2H])c([2H])c2c(c1[2H])c1c(-c3ccccc3-c3ccccc3)cccc1n2-c1ccc(-c2ccc(-c3nc(-c4ccccc4)nc(-c4ccccc4)n3)cc2)cc1 ; [2H]c1c([2H])c([2H])c2c(c1[2H])c1c(-c3ccccc3-c3ccccc3)cccc1n2-c1ccc(-c2cccc(-c3nc(-c4ccccc4)nc(-c4ccccc4)n3)c2)cc1 ; [2H]c1c([2H])c([2H])c2c(c1[2H])c1c(-c3ccccc3-c3ccccc3)cccc1n2-c1cccc(-c2ccc(-c3nc(-c4ccccc4)nc(-c4ccccc4)n3)cc2)c1]

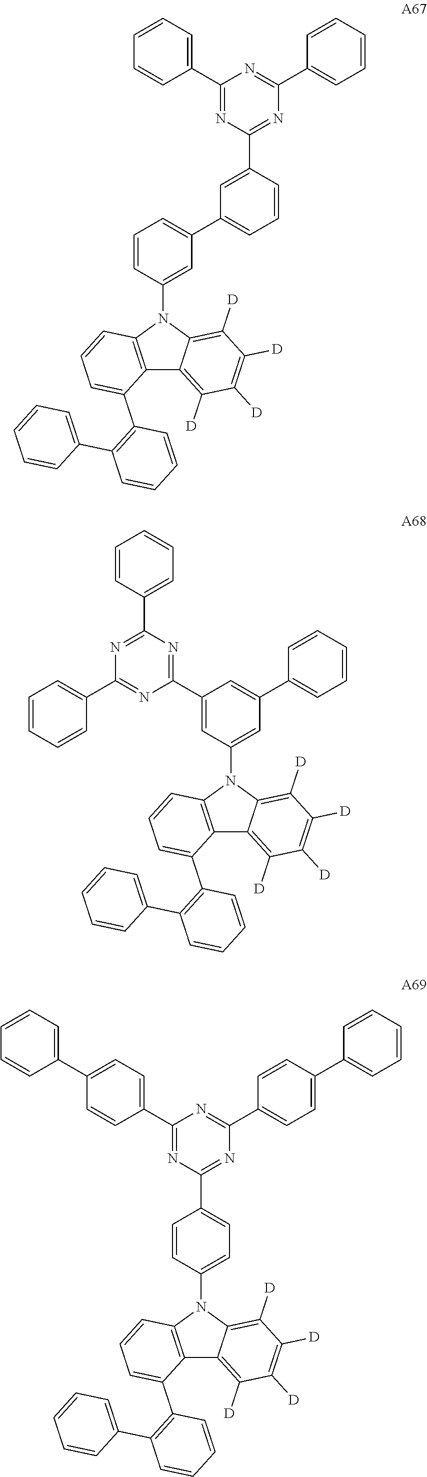

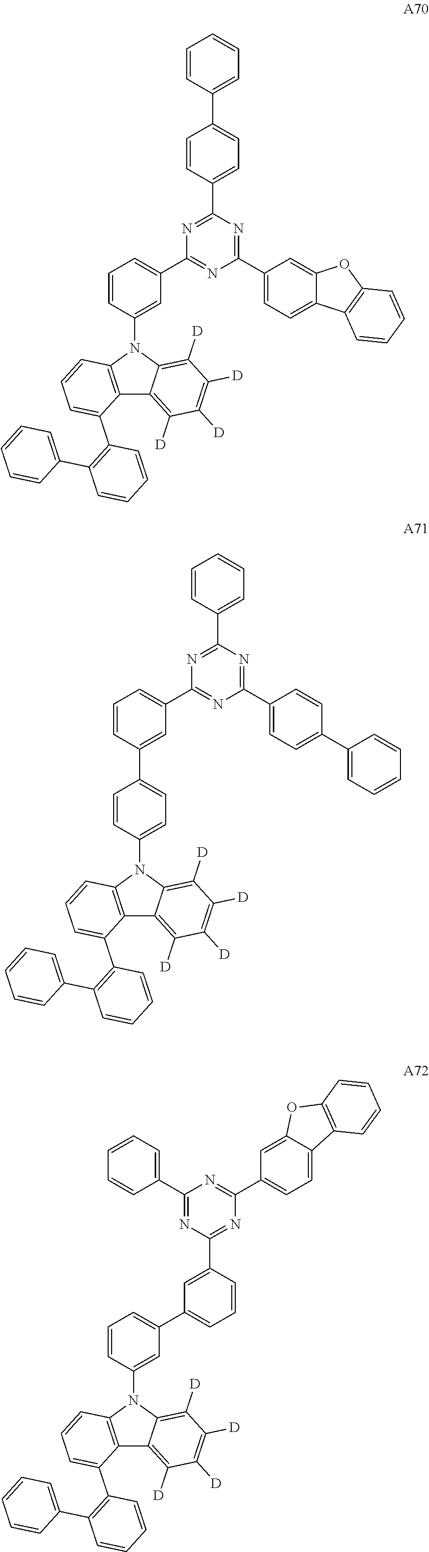 [SMILES: [2H]c1c([2H])c([2H])c2c(c1[2H])c1c(-c3ccccc3-c3ccccc3)cccc1n2-c1cccc(-c2nc(-c3ccc(-c4ccccc4)cc3)nc(-c3ccc4c(c3)oc3ccccc34)n2)c1 ; [2H]c1c([2H])c([2H])c2c(c1[2H])c1c(-c3ccccc3-c3ccccc3)cccc1n2-c1ccc(-c2cccc(-c3nc(-c4ccccc4)nc(-c4ccc(-c5ccccc5)cc4)n3)c2)cc1 ; [2H]c1c([2H])c([2H])c2c(c1[2H])c1c(-c3ccccc3-c3ccccc3)cccc1n2-c1cccc(-c2cccc(-c3nc(-c4ccccc4)nc(-c4ccc5c(c4)oc4ccccc45)n3)c2)c1]

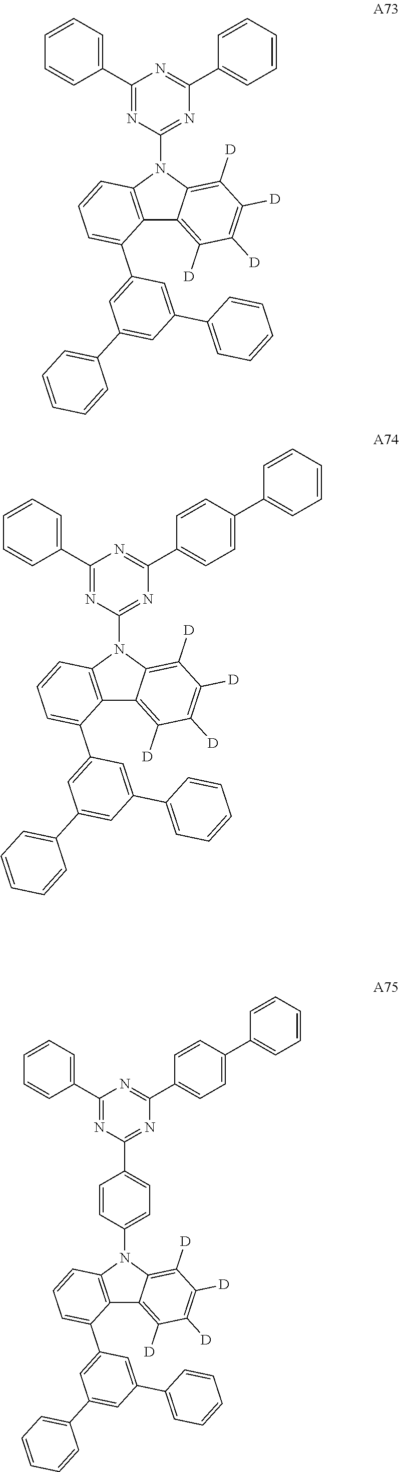 [SMILES: [2H]c1c([2H])c([2H])c2c(c1[2H])c1c(-c3cc(-c4ccccc4)cc(-c4ccccc4)c3)cccc1n2-c1nc(-c2ccccc2)nc(-c2ccccc2)n1 ; [2H]c1c([2H])c([2H])c2c(c1[2H])c1c(-c3cc(-c4ccccc4)cc(-c4ccccc4)c3)cccc1n2-c1nc(-c2ccccc2)nc(-c2ccc(-c3ccccc3)cc2)n1 ; [2H]c1c([2H])c([2H])c2c(c1[2H])c1c(-c3cc(-c4ccccc4)cc(-c4ccccc4)c3)cccc1n2-c1ccc(-c2nc(-c3ccccc3)nc(-c3ccc(-c4ccccc4)cc3)n2)cc1]

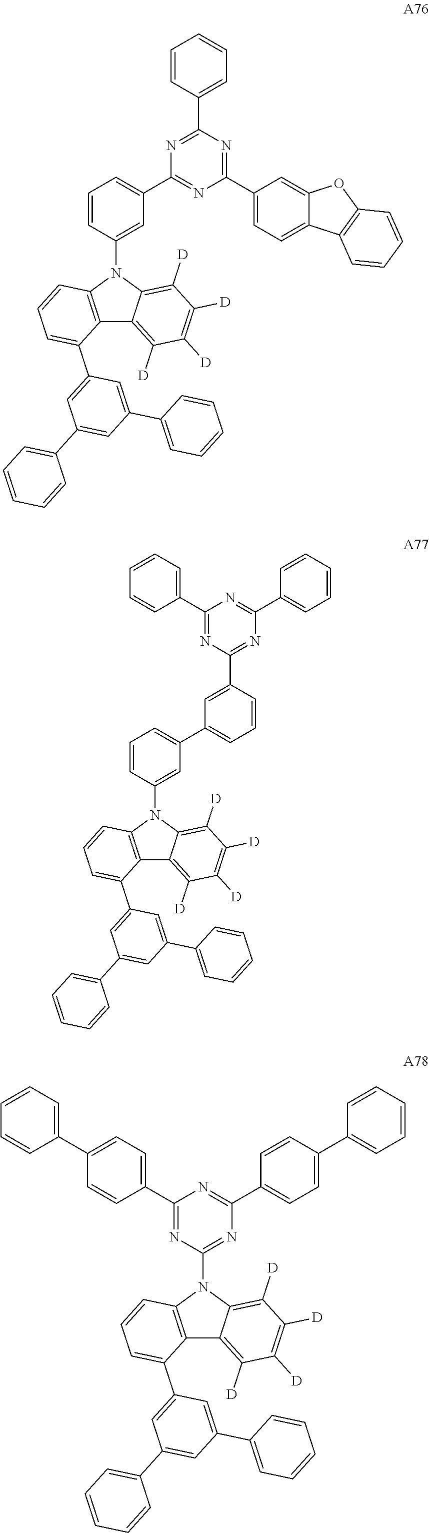 [SMILES: [2H]c1c([2H])c([2H])c2c(c1[2H])c1c(-c3cc(-c4ccccc4)cc(-c4ccccc4)c3)cccc1n2-c1cccc(-c2nc(-c3ccccc3)nc(-c3ccc4c(c3)oc3ccccc34)n2)c1 ; [2H]c1c([2H])c([2H])c2c(c1[2H])c1c(-c3cc(-c4ccccc4)cc(-c4ccccc4)c3)cccc1n2-c1cccc(-c2cccc(-c3nc(-c4ccccc4)nc(-c4ccccc4)n3)c2)c1 ; [2H]c1c([2H])c([2H])c2c(c1[2H])c1c(-c3cc(-c4ccccc4)cc(-c4ccccc4)c3)cccc1n2-c1nc(-c2ccc(-c3ccccc3)cc2)nc(-c2ccc(-c3ccccc3)cc2)n1]

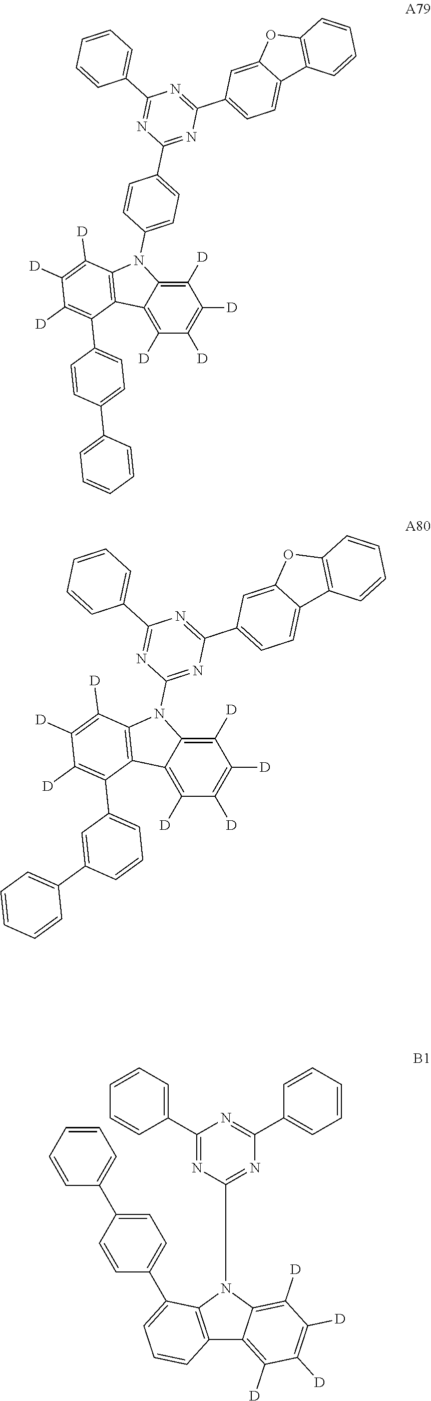 [SMILES: [2H]c1c([2H])c([2H])c2c(c1[2H])c1c(-c3ccc(-c4ccccc4)cc3)c([2H])c([2H])c([2H])c1n2-c1ccc(-c2nc(-c3ccccc3)nc(-c3ccc4c(c3)oc3ccccc34)n2)cc1 ; [2H]c1c([2H])c([2H])c2c(c1[2H])c1c(-c3cccc(-c4ccccc4)c3)c([2H])c([2H])c([2H])c1n2-c1nc(-c2ccccc2)nc(-c2ccc3c(c2)oc2ccccc23)n1 ; [2H]c1c([2H])c([2H])c2c(c1[2H])c1cccc(-c3ccc(-c4ccccc4)cc3)c1n2-c1nc(-c2ccccc2)nc(-c2ccccc2)n1]

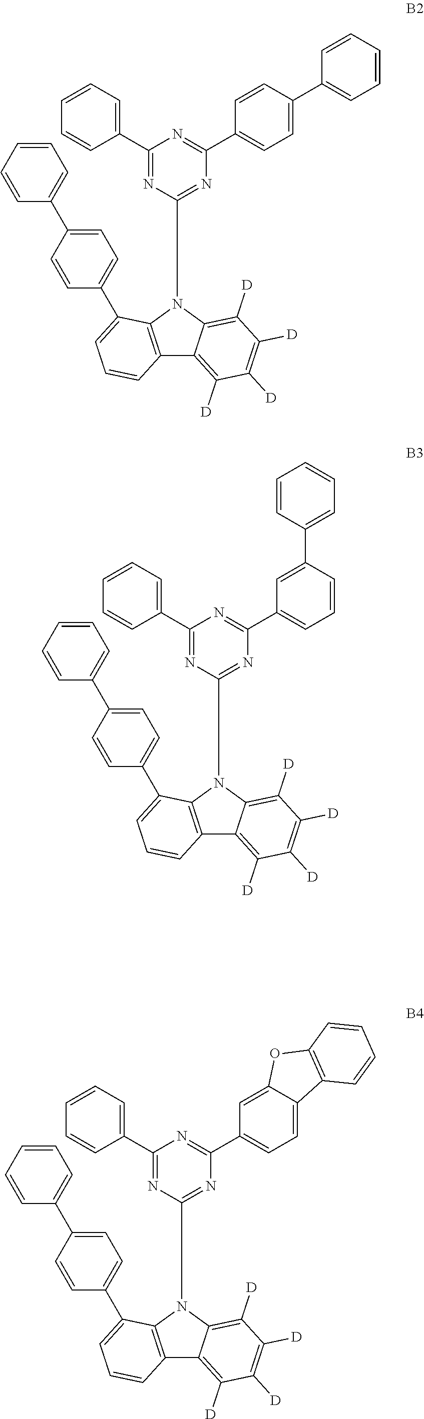 [SMILES: [2H]c1c([2H])c([2H])c2c(c1[2H])c1cccc(-c3ccc(-c4ccccc4)cc3)c1n2-c1nc(-c2ccccc2)nc(-c2ccc(-c3ccccc3)cc2)n1 ; [2H]c1c([2H])c([2H])c2c(c1[2H])c1cccc(-c3ccc(-c4ccccc4)cc3)c1n2-c1nc(-c2ccccc2)nc(-c2cccc(-c3ccccc3)c2)n1 ; [2H]c1c([2H])c([2H])c2c(c1[2H])c1cccc(-c3ccc(-c4ccccc4)cc3)c1n2-c1nc(-c2ccccc2)nc(-c2ccc3c(c2)oc2ccccc23)n1]

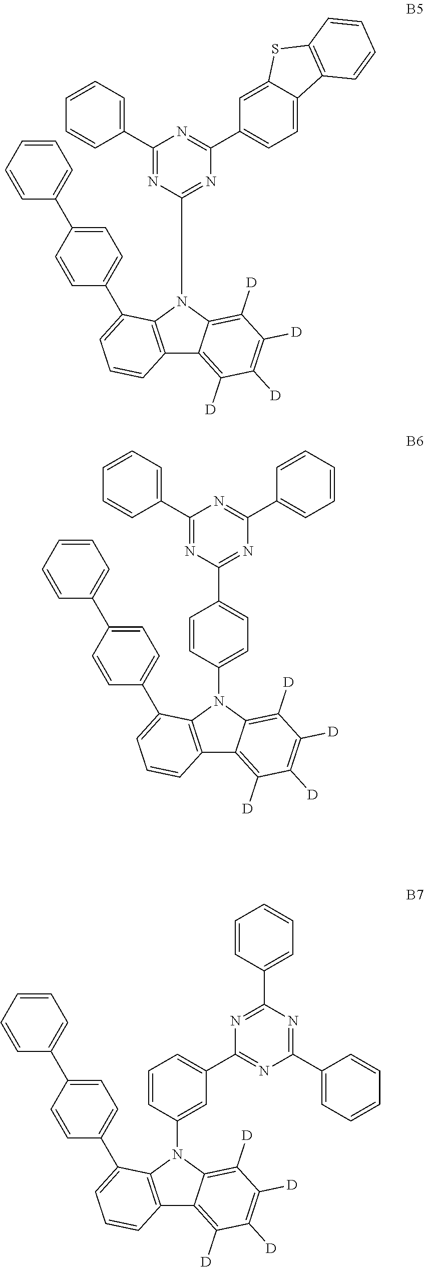 [SMILES: [2H]c1c([2H])c([2H])c2c(c1[2H])c1cccc(-c3ccc(-c4ccccc4)cc3)c1n2-c1nc(-c2ccccc2)nc(-c2ccc3c(c2)sc2ccccc23)n1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccccc3)n2)cc1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccccc3)n2)c1]

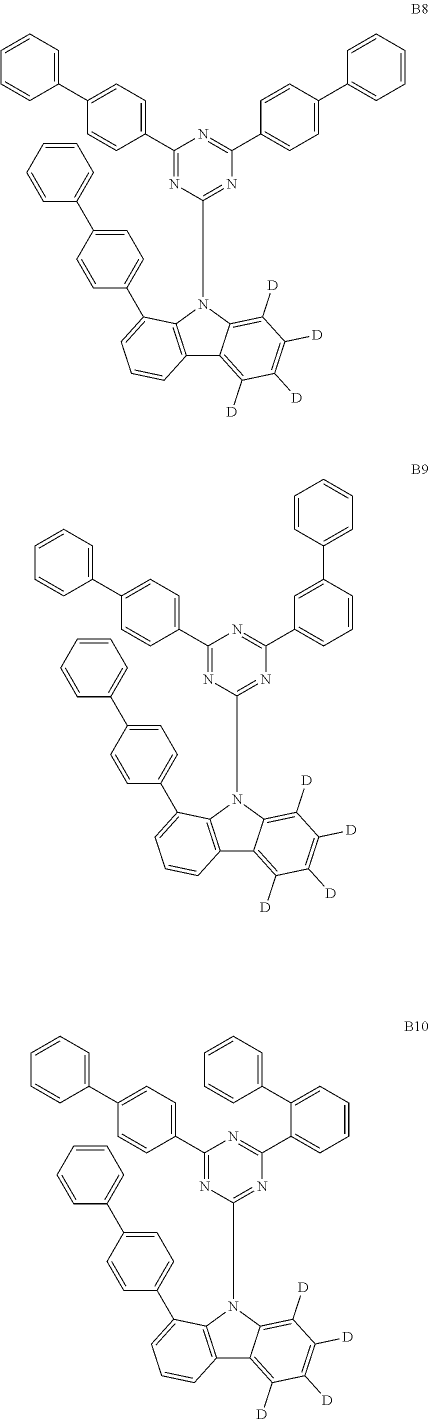 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc(-c3ccccc3)c2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccccc2-c2ccccc2)n1]

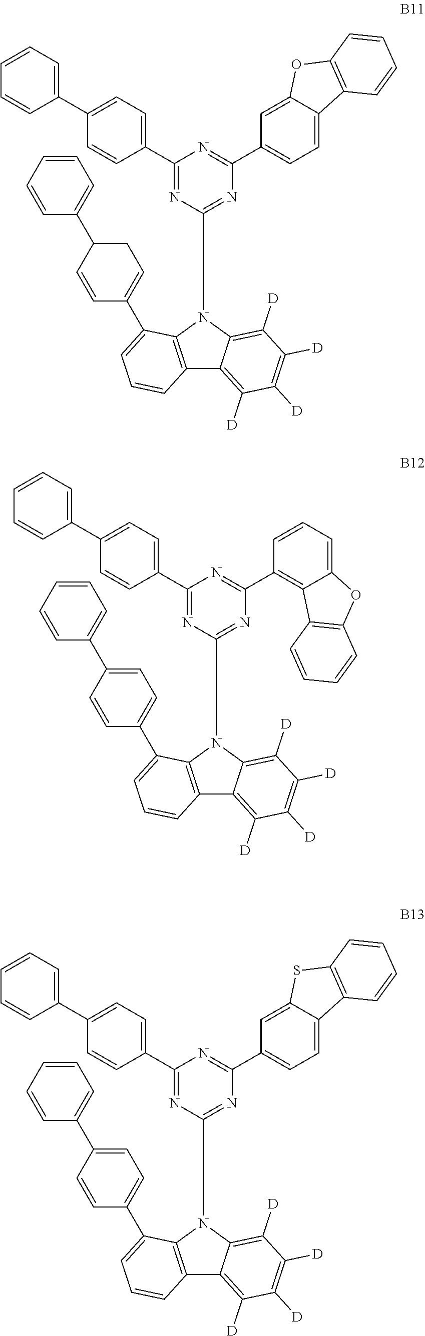 [SMILES: [2H]c1c([2H])c([2H])c2c(c1[2H])c1cccc(C3=CCC(c4ccccc4)C=C3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc3oc4ccccc4c23)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc3c(c2)sc2ccccc23)n1]

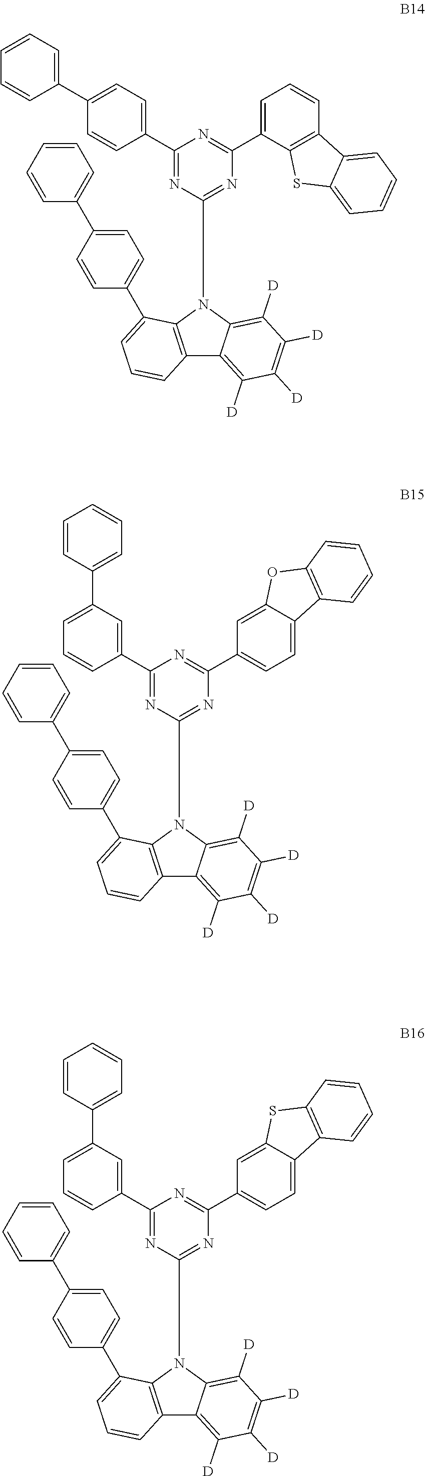 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2cccc3c2sc2ccccc23)n1 ; [2H]c1c([2H])c([2H])c2c(c1[2H])c1cccc(-c3ccc(-c4ccccc4)cc3)c1n2-c1nc(-c2cccc(-c3ccccc3)c2)nc(-c2ccc3c(c2)oc2ccccc23)n1 ; [2H]c1c([2H])c([2H])c2c(c1[2H])c1cccc(-c3ccc(-c4ccccc4)cc3)c1n2-c1nc(-c2cccc(-c3ccccc3)c2)nc(-c2ccc3c(c2)sc2ccccc23)n1]

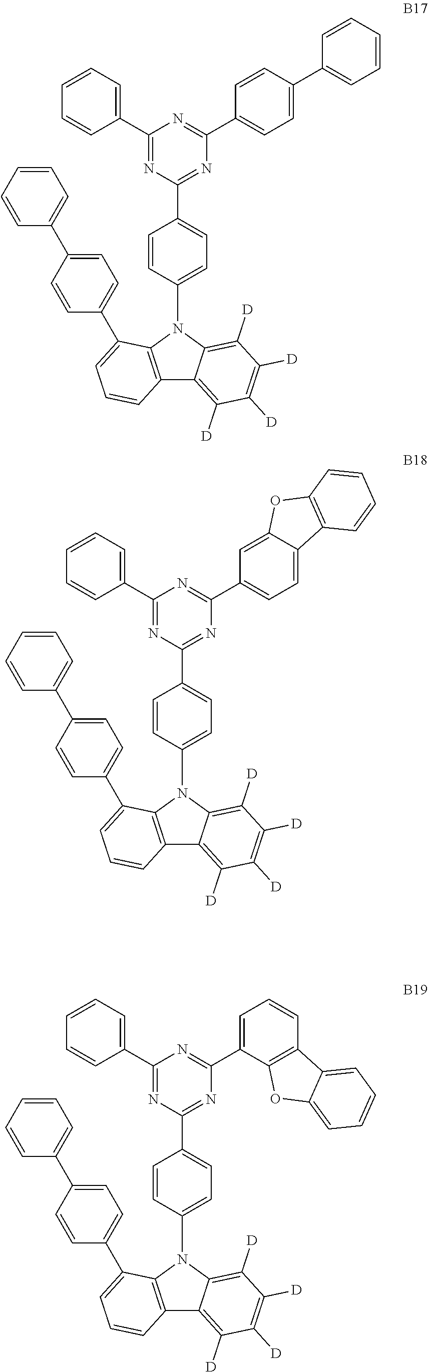 [SMILES: [2H]c1c([2H])c([2H])c2c(c1[2H])c1cccc(-c3ccc(-c4ccccc4)cc3)c1n2-c1ccc(-c2nc(-c3ccccc3)nc(-c3ccc(-c4ccccc4)cc3)n2)cc1 ; [2H]c1c([2H])c([2H])c2c(c1[2H])c1cccc(-c3ccc(-c4ccccc4)cc3)c1n2-c1ccc(-c2nc(-c3ccccc3)nc(-c3ccc4c(c3)oc3ccccc34)n2)cc1 ; [2H]c1c([2H])c([2H])c2c(c1[2H])c1cccc(-c3ccc(-c4ccccc4)cc3)c1n2-c1ccc(-c2nc(-c3ccccc3)nc(-c3cccc4c3oc3ccccc34)n2)cc1]

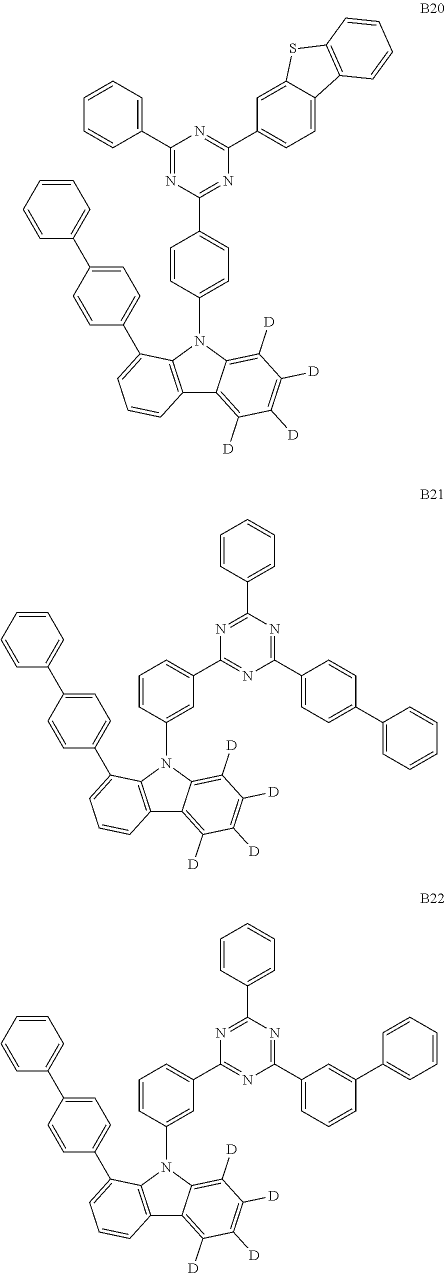 [SMILES: [2H]c1c([2H])c([2H])c2c(c1[2H])c1cccc(-c3ccc(-c4ccccc4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3ccc(-c4ccccc4)cc3)c1n2-c1cccc(-c2nc(-c3ccccc3)nc(-c3cccc(-c4ccccc4)c3)n2)c1]

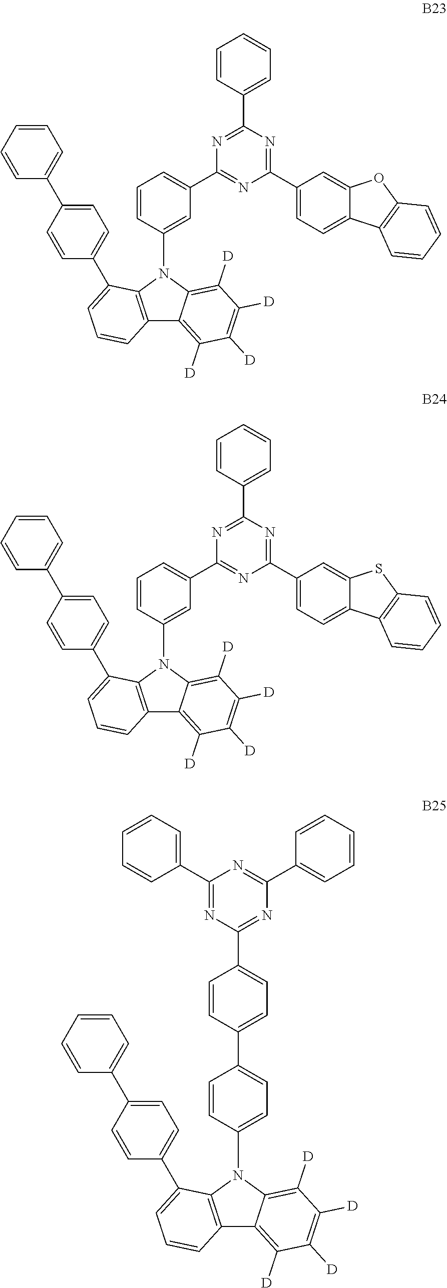 [SMILES: [2H]c1c([2H])c([2H])c2c(c1[2H])c1cccc(-c3ccc(-c4ccccc4)cc3)c1n2-c1cccc(-c2nc(-c3ccccc3)nc(-c3ccc4c(c3)oc3ccccc34)n2)c1 ; [2H]c1c([2H])c([2H])c2c(c1[2H])c1cccc(-c3ccc(-c4ccccc4)cc3)c1n2-c1cccc(-c2nc(-c3ccccc3)nc(-c3ccc4c(c3)sc3ccccc34)n2)c1 ; [2H]c1c([2H])c([2H])c2c(c1[2H])c1cccc(-c3ccc(-c4ccccc4)cc3)c1n2-c1ccc(-c2ccc(-c3nc(-c4ccccc4)nc(-c4ccccc4)n3)cc2)cc1]

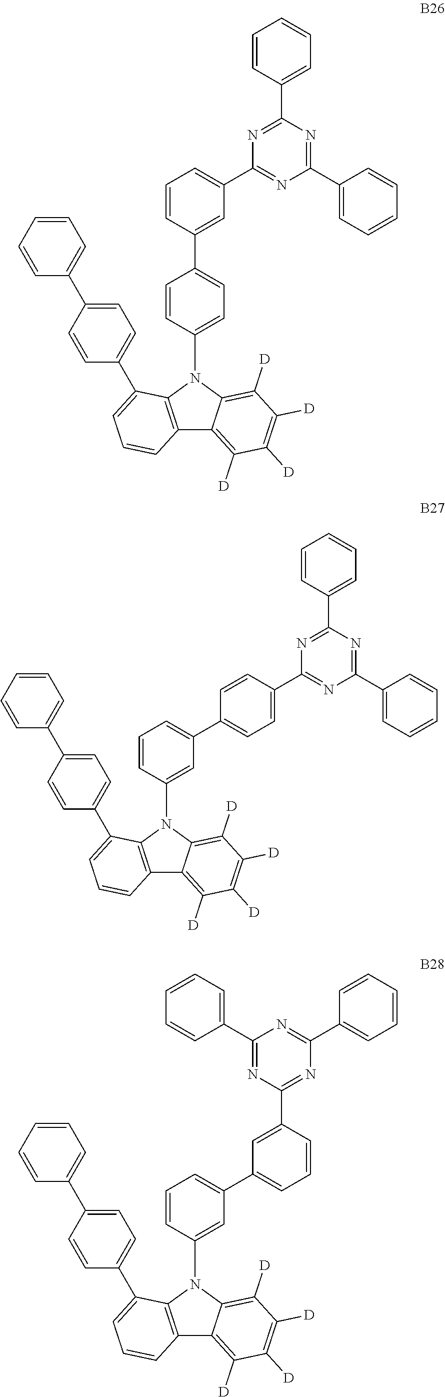 [SMILES: [2H]c1c([2H])c([2H])c2c(c1[2H])c1cccc(-c3ccc(-c4ccccc4)cc3)c1n2-c1ccc(-c2cccc(-c3nc(-c4ccccc4)nc(-c4ccccc4)n3)c2)cc1 ; [2H]c1c([2H])c([2H])c2c(c1[2H])c1cccc(-c3ccc(-c4ccccc4)cc3)c1n2-c1cccc(-c2ccc(-c3nc(-c4ccccc4)nc(-c4ccccc4)n3)cc2)c1 ; [2H]c1c([2H])c([2H])c2c(c1[2H])c1cccc(-c3ccc(-c4ccccc4)cc3)c1n2-c1cccc(-c2cccc(-c3nc(-c4ccccc4)nc(-c4ccccc4)n3)c2)c1]

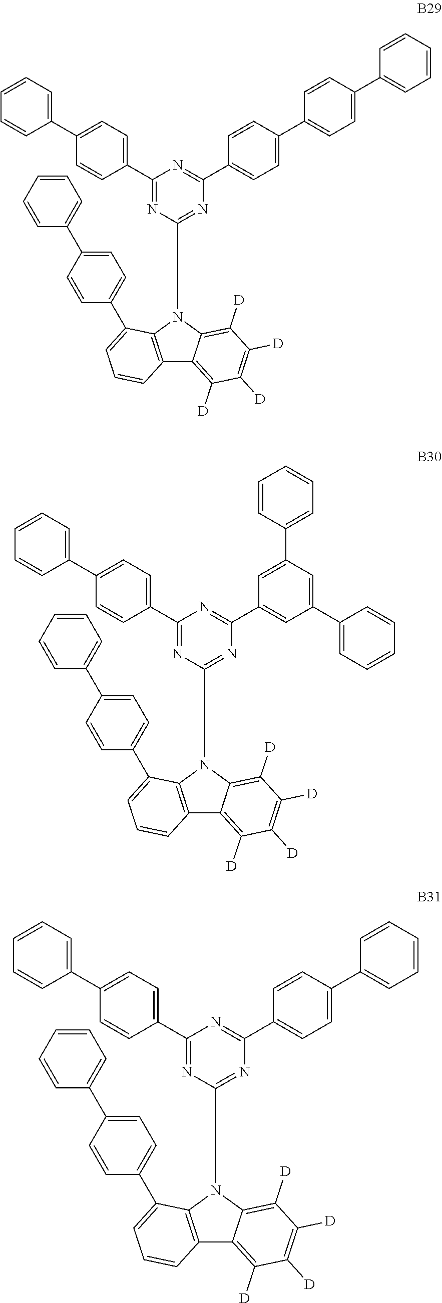 [SMILES: [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccc(-c4ccccc4)cc3)cc2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2cc(-c3ccccc3)cc(-c3ccccc3)c2)n1 ; [2H]c1c([2H])c([2H])c2c(c1[2H])c1cccc(-c3ccc(-c4ccccc4)cc3)c1n2-c1nc(-c2ccc(-c3ccccc3)cc2)nc(-c2ccc(-c3ccccc3)cc2)n1]

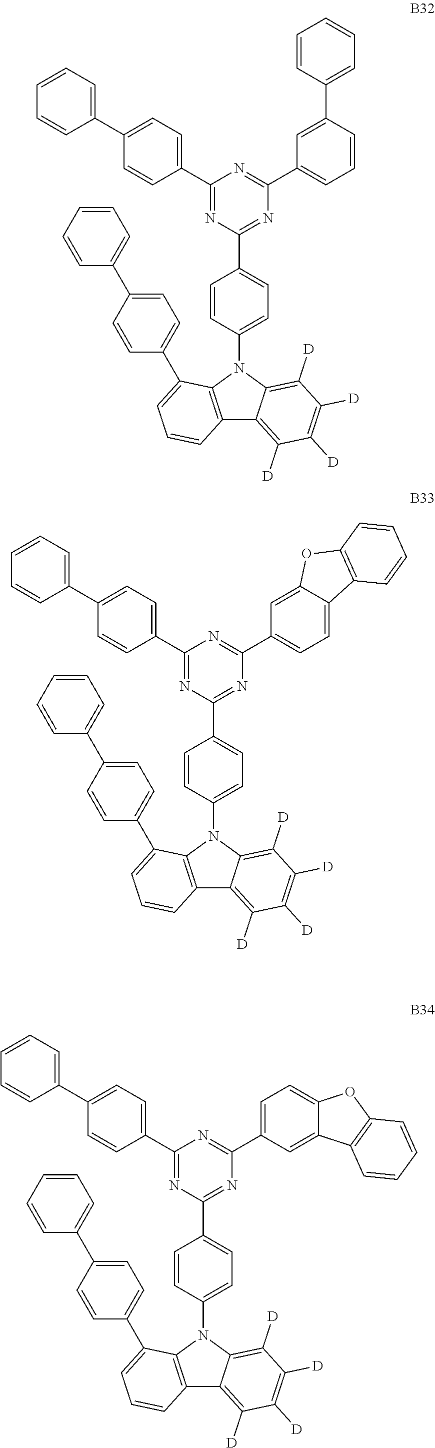 [SMILES: [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3cccc(-c4ccccc4)c3)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4c(c3)oc3ccccc34)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4oc5ccccc5c4c3)n2)cc1]

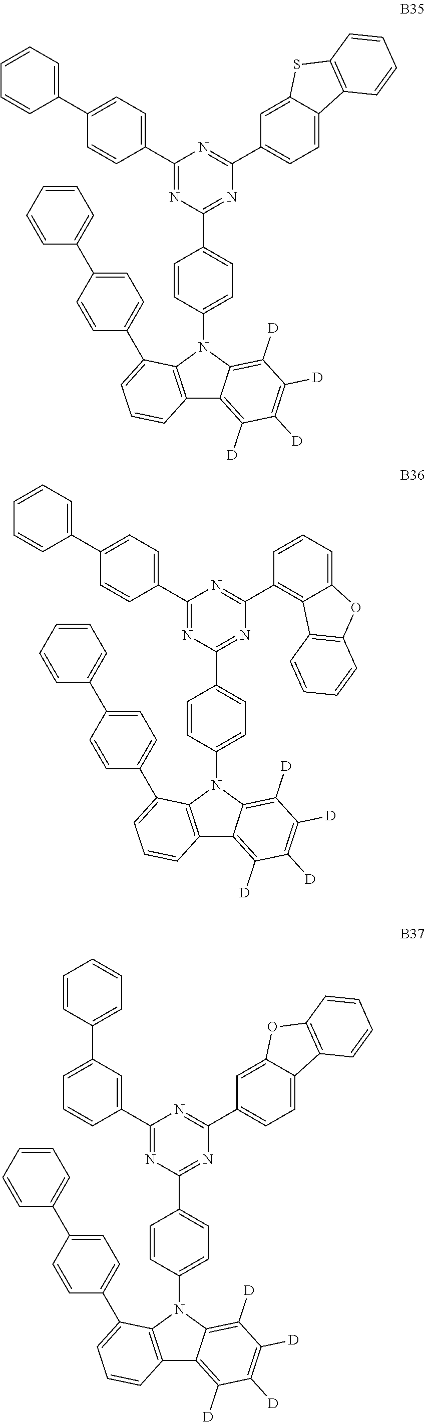 [SMILES: [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3ccc4c(c3)sc3ccccc34)n2)cc1 ; [2H]c1c([2H])c([2H])c2c(c1[2H])c1cccc(-c3ccc(-c4ccccc4)cc3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3ccc(-c4ccccc4)cc3)c1n2-c1ccc(-c2nc(-c3cccc(-c4ccccc4)c3)nc(-c3ccc4c(c3)oc3ccccc34)n2)cc1]

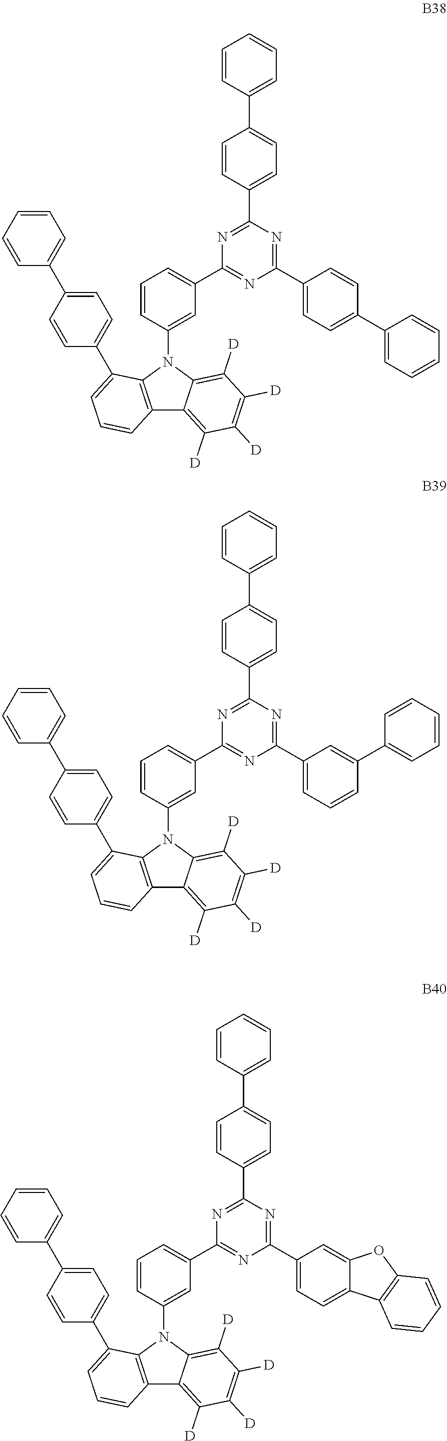 [SMILES: [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3ccc(-c4ccccc4)cc3)n2)c1 ; [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3cccc(-c4ccccc4)c3)n2)c1 ; [2H]c1c([2H])c([2H])c2c(c1[2H])c1cccc(-c3ccc(-c4ccccc4)cc3)c1n2-c1cccc(-c2nc(-c3ccc(-c4ccccc4)cc3)nc(-c3ccc4c(c3)oc3ccccc34)n2)c1]

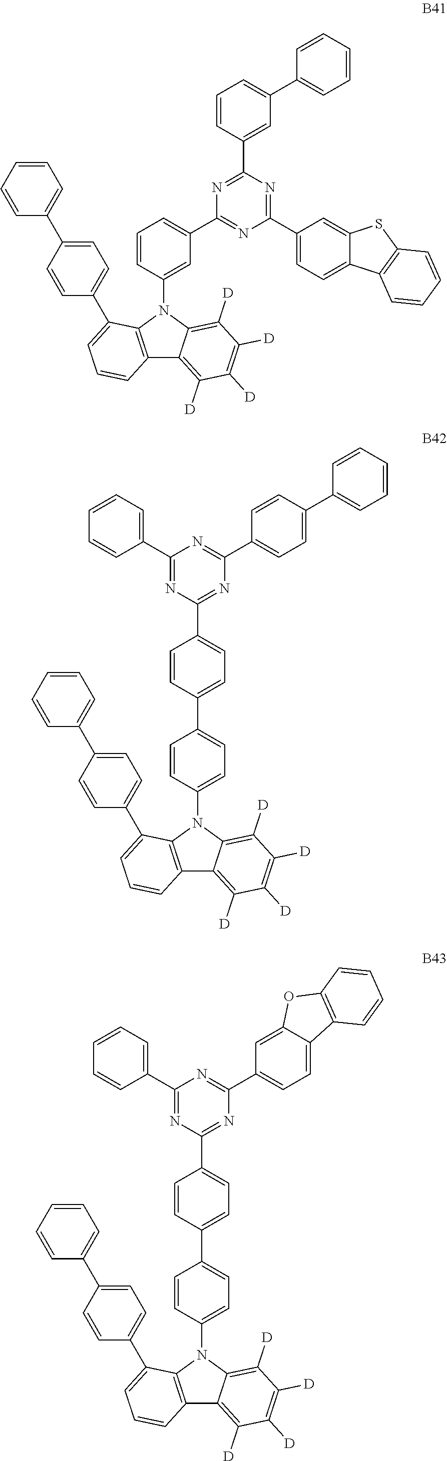 [SMILES: [2H]c1c([2H])c([2H])c2c(c1[2H])c1cccc(-c3ccc(-c4ccccc4)cc3)c1n2-c1cccc(-c2nc(-c3cccc(-c4ccccc4)c3)nc(-c3ccc4c(c3)sc3ccccc34)n2)c1 ; [2H]c1c([2H])c([2H])c2c(c1[2H])c1cccc(-c3ccc(-c4ccccc4)cc3)c1n2-c1ccc(-c2ccc(-c3nc(-c4ccccc4)nc(-c4ccc(-c5ccccc5)cc4)n3)cc2)cc1 ; [2H]c1c([2H])c([2H])c2c(c1[2H])c1cccc(-c3ccc(-c4ccccc4)cc3)c1n2-c1ccc(-c2ccc(-c3nc(-c4ccccc4)nc(-c4ccc5c(c4)oc4ccccc45)n3)cc2)cc1]

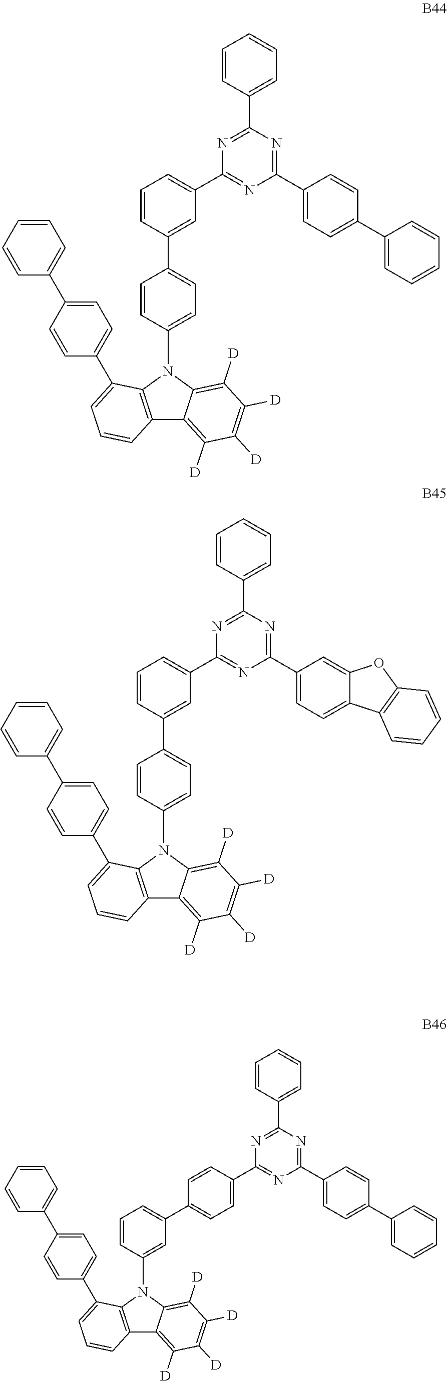 [SMILES: [2H]c1c([2H])c([2H])c2c(c1[2H])c1cccc(-c3ccc(-c4ccccc4)cc3)c1n2-c1ccc(-c2cccc(-c3nc(-c4ccccc4)nc(-c4ccc(-c5ccccc5)cc4)n3)c2)cc1 ; [2H]c1c([2H])c([2H])c2c(c1[2H])c1cccc(-c3ccc(-c4ccccc4)cc3)c1n2-c1ccc(-c2cccc(-c3nc(-c4ccccc4)nc(-c4ccc5c(c4)oc4ccccc45)n3)c2)cc1 ; [2H]c1c([2H])c([2H])c2c(c1[2H])c1cccc(-c3ccc(-c4ccccc4)cc3)c1n2-c1cccc(-c2ccc(-c3nc(-c4ccccc4)nc(-c4ccc(-c5ccccc5)cc4)n3)cc2)c1]

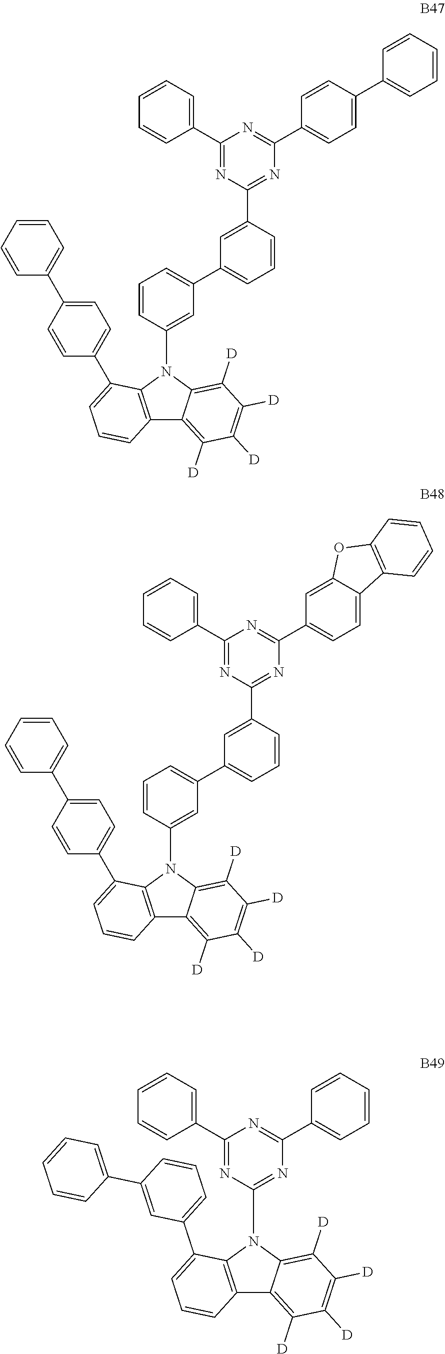 [SMILES: [2H]c1c([2H])c([2H])c2c(c1[2H])c1cccc(-c3ccc(-c4ccccc4)cc3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3ccc(-c4ccccc4)cc3)c1n2-c1cccc(-c2cccc(-c3nc(-c4ccccc4)nc(-c4ccc5c(c4)oc4ccccc45)n3)c2)c1 ; [2H]c1c([2H])c([2H])c2c(c1[2H])c1cccc(-c3cccc(-c4ccccc4)c3)c1n2-c1nc(-c2ccccc2)nc(-c2ccccc2)n1]

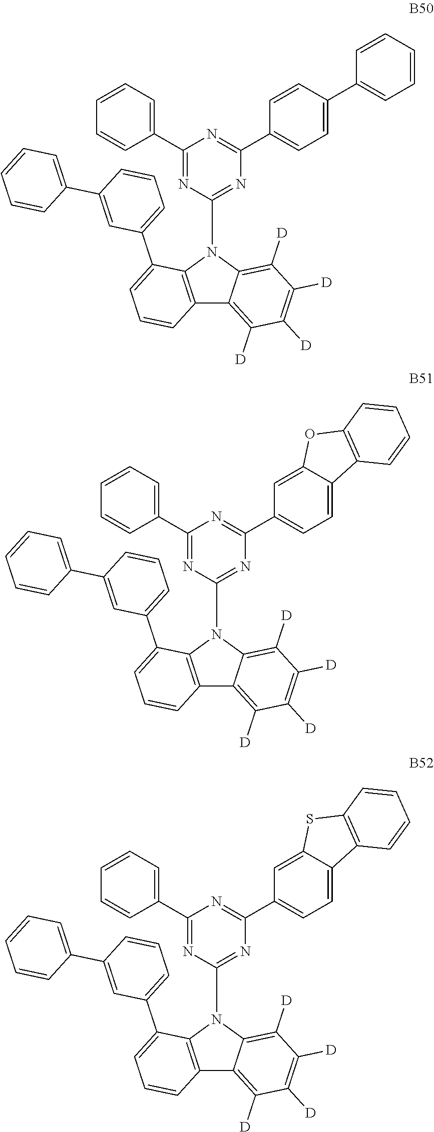 [SMILES: [2H]c1c([2H])c([2H])c2c(c1[2H])c1cccc(-c3cccc(-c4ccccc4)c3)c1n2-c1nc(-c2ccccc2)nc(-c2ccc(-c3ccccc3)cc2)n1 ; [2H]c1c([2H])c([2H])c2c(c1[2H])c1cccc(-c3cccc(-c4ccccc4)c3)c1n2-c1nc(-c2ccccc2)nc(-c2ccc3c(c2)oc2ccccc23)n1 ; [2H]c1c([2H])c([2H])c2c(c1[2H])c1cccc(-c3cccc(-c4ccccc4)c3)c1n2-c1nc(-c2ccccc2)nc(-c2ccc3c(c2)sc2ccccc23)n1]

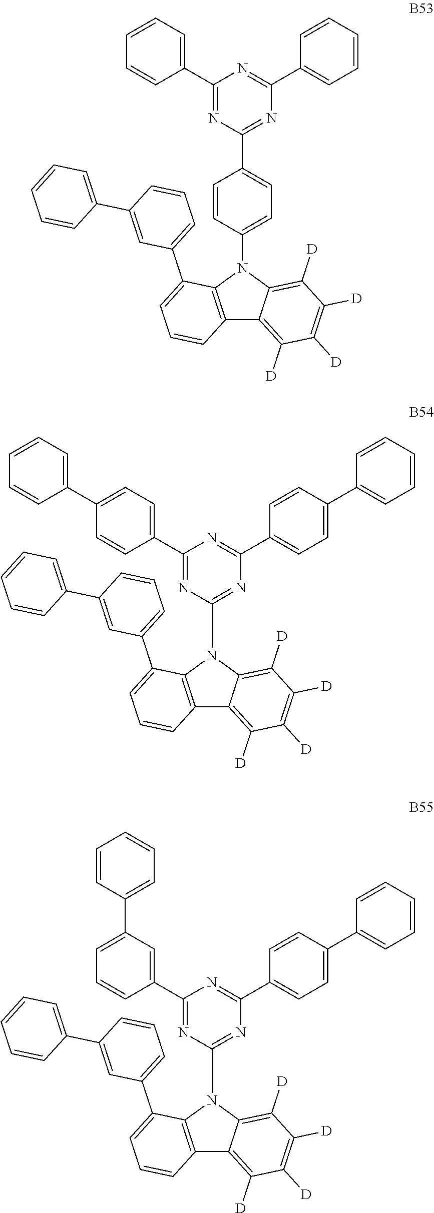 [SMILES: [2H]c1c([2H])c([2H])c2c(c1[2H])c1cccc(-c3cccc(-c4ccccc4)c3)c1n2-c1ccc(-c2nc(-c3ccccc3)nc(-c3ccccc3)n2)cc1 ; [2H]c1c([2H])c([2H])c2c(c1[2H])c1cccc(-c3cccc(-c4ccccc4)c3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3cccc(-c4ccccc4)c3)c1n2-c1nc(-c2ccc(-c3ccccc3)cc2)nc(-c2cccc(-c3ccccc3)c2)n1]

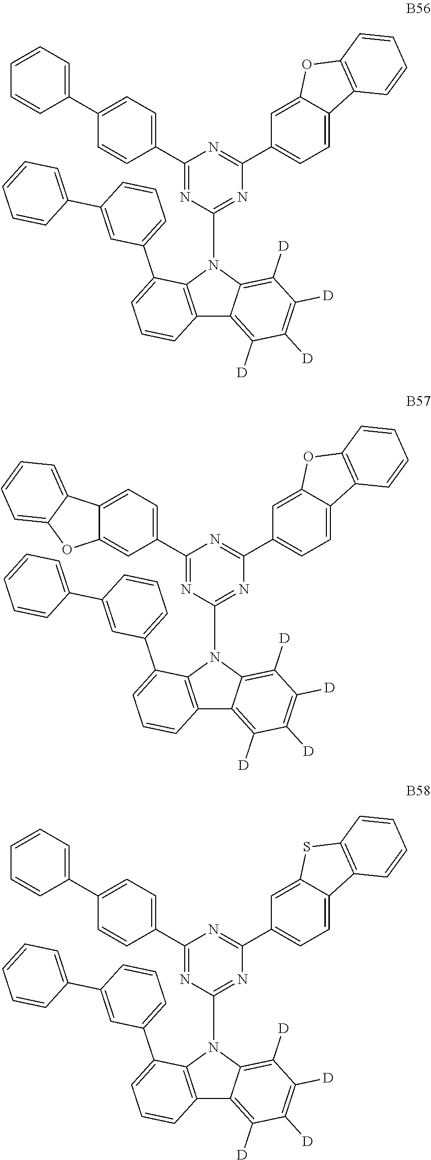 [SMILES: [2H]c1c([2H])c([2H])c2c(c1[2H])c1cccc(-c3cccc(-c4ccccc4)c3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3cccc(-c4ccccc4)c3)c1n2-c1nc(-c2ccc3c(c2)oc2ccccc23)nc(-c2ccc3c(c2)oc2ccccc23)n1 ; [2H]c1c([2H])c([2H])c2c(c1[2H])c1cccc(-c3cccc(-c4ccccc4)c3)c1n2-c1nc(-c2ccc(-c3ccccc3)cc2)nc(-c2ccc3c(c2)sc2ccccc23)n1]

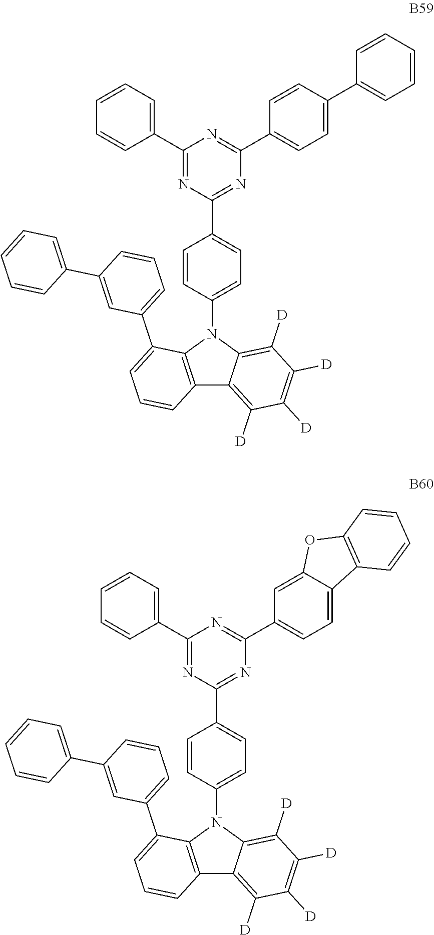 [SMILES: [2H]c1c([2H])c([2H])c2c(c1[2H])c1cccc(-c3cccc(-c4ccccc4)c3)c1n2-c1ccc(-c2nc(-c3ccccc3)nc(-c3ccc(-c4ccccc4)cc3)n2)cc1 ; [2H]c1c([2H])c([2H])c2c(c1[2H])c1cccc(-c3cccc(-c4ccccc4)c3)c1n2-c1ccc(-c2nc(-c3ccccc3)nc(-c3ccc4c(c3)oc3ccccc34)n2)cc1]

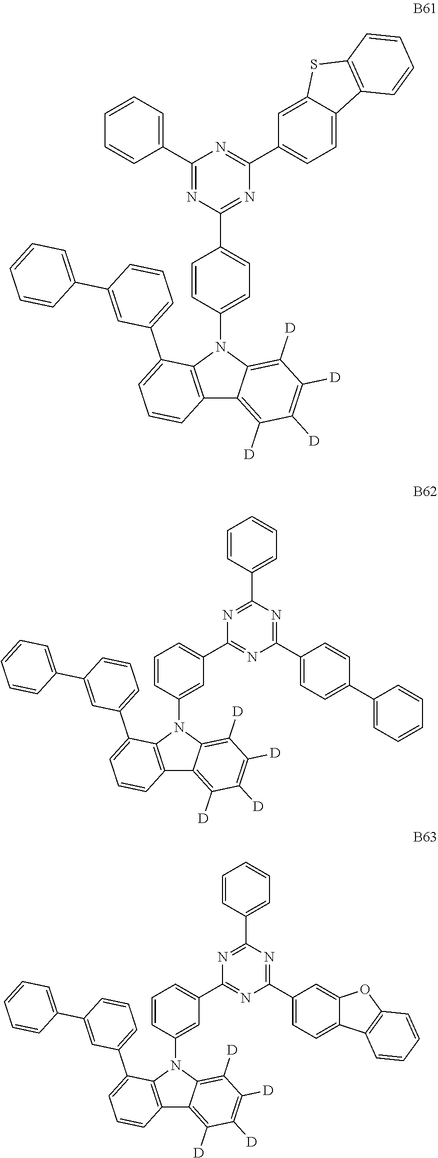 [SMILES: [2H]c1c([2H])c([2H])c2c(c1[2H])c1cccc(-c3cccc(-c4ccccc4)c3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3cccc(-c4ccccc4)c3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3cccc(-c4ccccc4)c3)c1n2-c1cccc(-c2nc(-c3ccccc3)nc(-c3ccc4c(c3)oc3ccccc34)n2)c1]

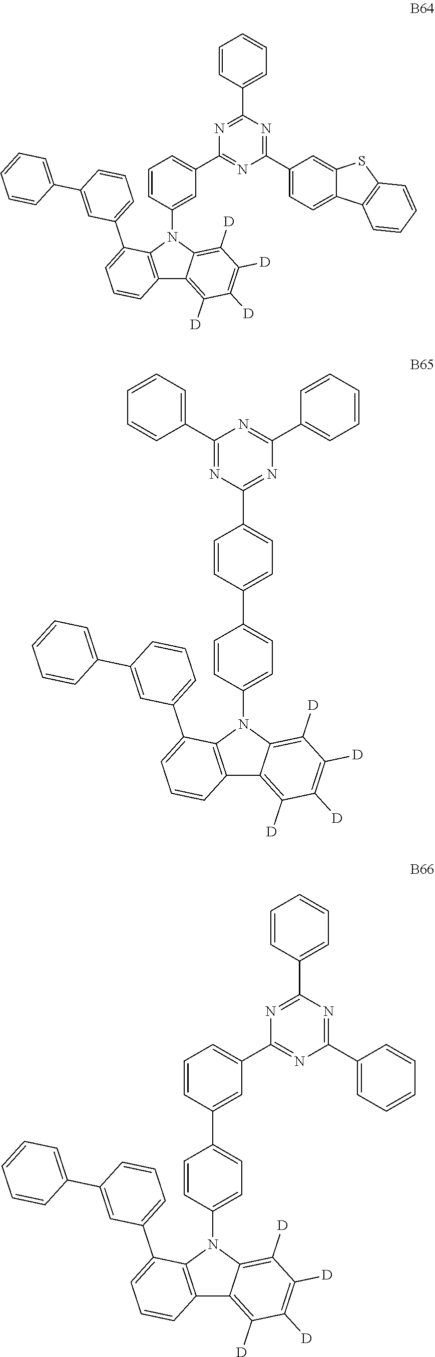 [SMILES: [2H]c1c([2H])c([2H])c2c(c1[2H])c1cccc(-c3cccc(-c4ccccc4)c3)c1n2-c1cccc(-c2nc(-c3ccccc3)nc(-c3ccc4c(c3)sc3ccccc34)n2)c1 ; [2H]c1c([2H])c([2H])c2c(c1[2H])c1cccc(-c3cccc(-c4ccccc4)c3)c1n2-c1ccc(-c2ccc(-c3nc(-c4ccccc4)nc(-c4ccccc4)n3)cc2)cc1 ; [2H]c1c([2H])c([2H])c2c(c1[2H])c1cccc(-c3cccc(-c4ccccc4)c3)c1n2-c1ccc(-c2cccc(-c3nc(-c4ccccc4)nc(-c4ccccc4)n3)c2)cc1]

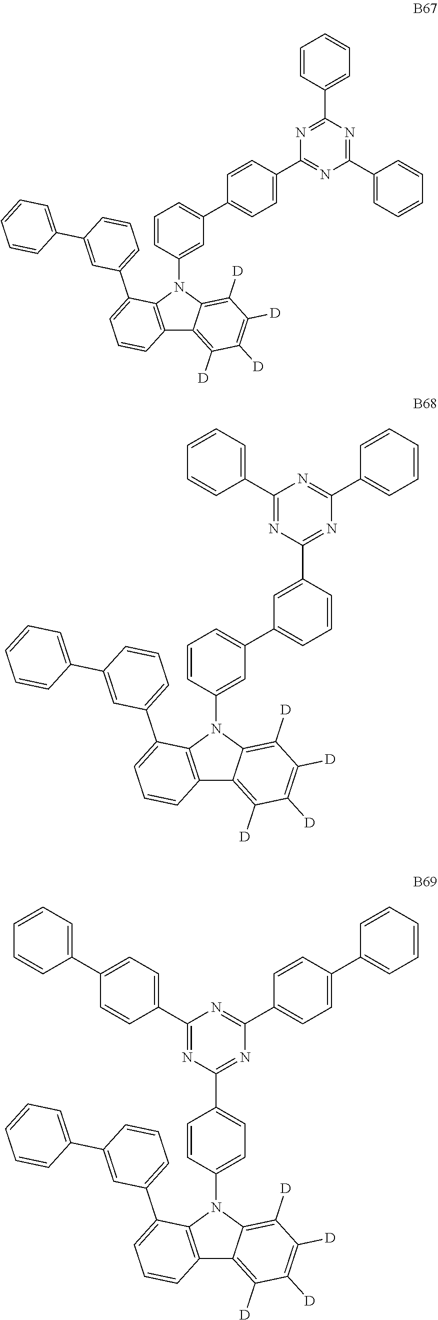 [SMILES: [2H]c1c([2H])c([2H])c2c(c1[2H])c1cccc(-c3cccc(-c4ccccc4)c3)c1n2-c1cccc(-c2ccc(-c3nc(-c4ccccc4)nc(-c4ccccc4)n3)cc2)c1 ; [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccccc4)n3)c2)c1 ; [2H]c1c([2H])c([2H])c2c(c1[2H])c1cccc(-c3cccc(-c4ccccc4)c3)c1n2-c1ccc(-c2nc(-c3ccc(-c4ccccc4)cc3)nc(-c3ccc(-c4ccccc4)cc3)n2)cc1]

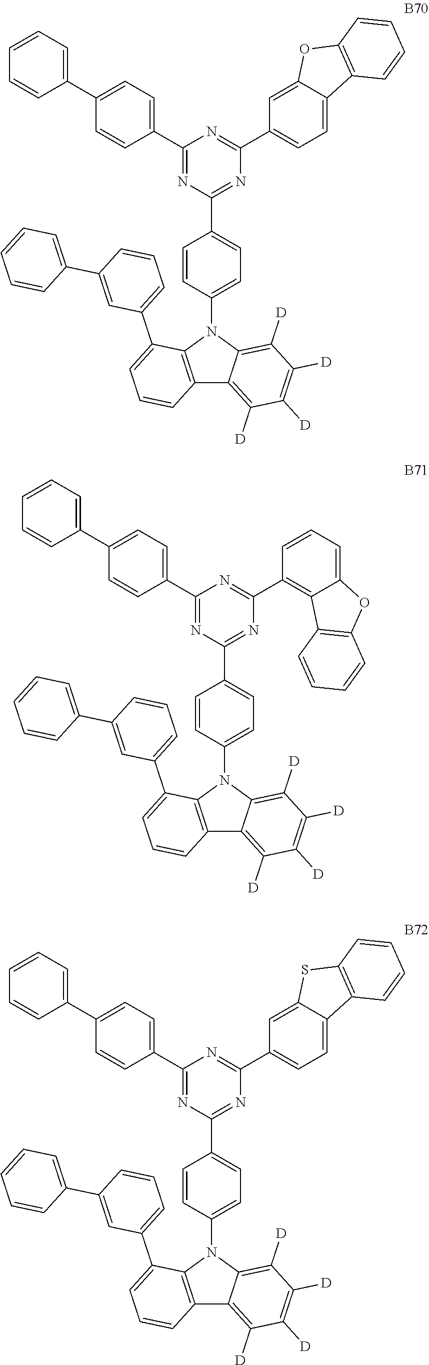 [SMILES: [2H]c1c([2H])c([2H])c2c(c1[2H])c1cccc(-c3cccc(-c4ccccc4)c3)c1n2-c1ccc(-c2nc(-c3ccc(-c4ccccc4)cc3)nc(-c3ccc4c(c3)oc3ccccc34)n2)cc1 ; [2H]c1c([2H])c([2H])c2c(c1[2H])c1cccc(-c3cccc(-c4ccccc4)c3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3cccc(-c4ccccc4)c3)c1n2-c1ccc(-c2nc(-c3ccc(-c4ccccc4)cc3)nc(-c3ccc4c(c3)sc3ccccc34)n2)cc1]

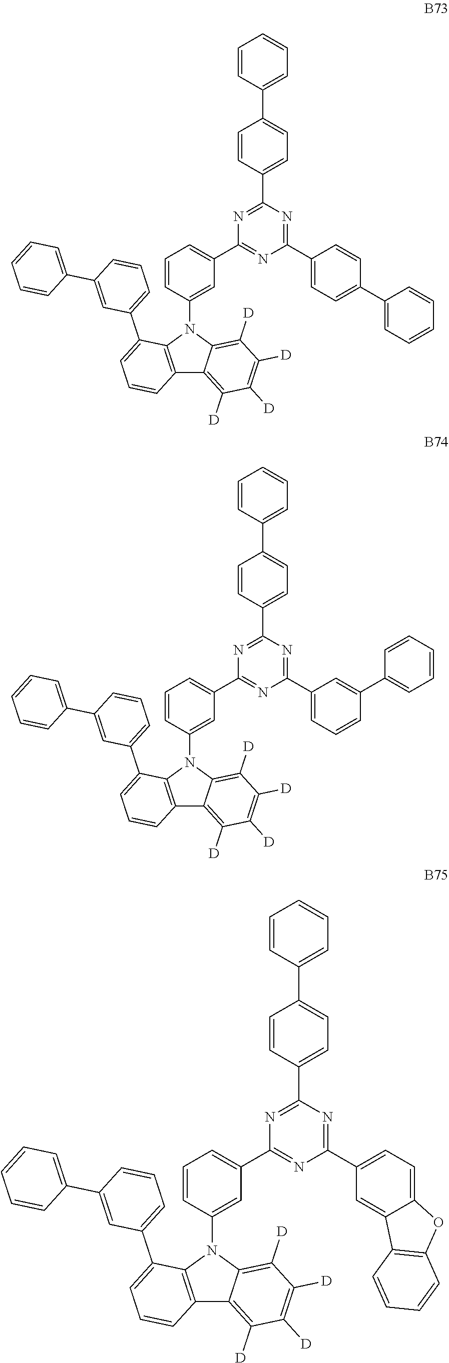 [SMILES: [2H]c1c([2H])c([2H])c2c(c1[2H])c1cccc(-c3cccc(-c4ccccc4)c3)c1n2-c1cccc(-c2nc(-c3ccc(-c4ccccc4)cc3)nc(-c3ccc(-c4ccccc4)cc3)n2)c1 ; [2H]c1c([2H])c([2H])c2c(c1[2H])c1cccc(-c3cccc(-c4ccccc4)c3)c1n2-c1cccc(-c2nc(-c3ccc(-c4ccccc4)cc3)nc(-c3cccc(-c4ccccc4)c3)n2)c1 ; [2H]c1c([2H])c([2H])c2c(c1[2H])c1cccc(-c3cccc(-c4ccccc4)c3)c1n2-c1cccc(-c2nc(-c3ccc(-c4ccccc4)cc3)nc(-c3ccc4oc5ccccc5c4c3)n2)c1]

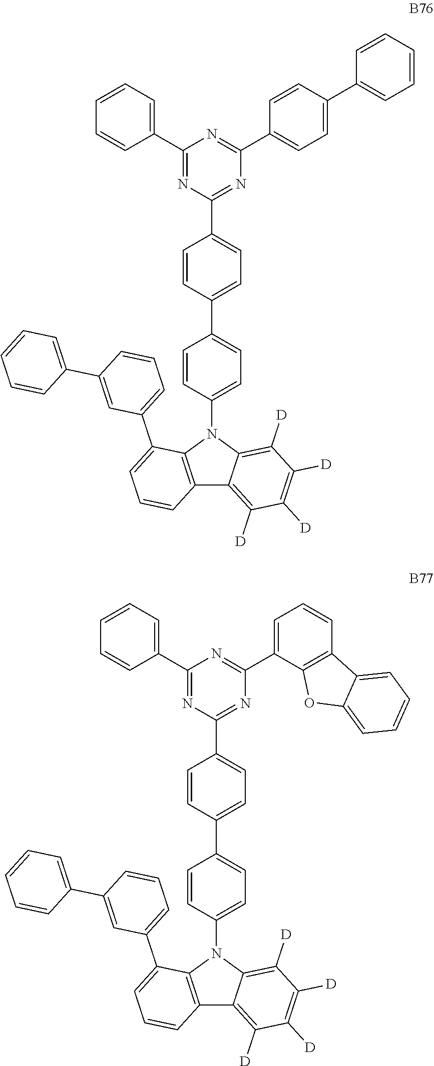 [SMILES: [2H]c1c([2H])c([2H])c2c(c1[2H])c1cccc(-c3cccc(-c4ccccc4)c3)c1n2-c1ccc(-c2ccc(-c3nc(-c4ccccc4)nc(-c4ccc(-c5ccccc5)cc4)n3)cc2)cc1 ; [2H]c1c([2H])c([2H])c2c(c1[2H])c1cccc(-c3cccc(-c4ccccc4)c3)c1n2-c1ccc(-c2ccc(-c3nc(-c4ccccc4)nc(-c4cccc5c4oc4ccccc45)n3)cc2)cc1]

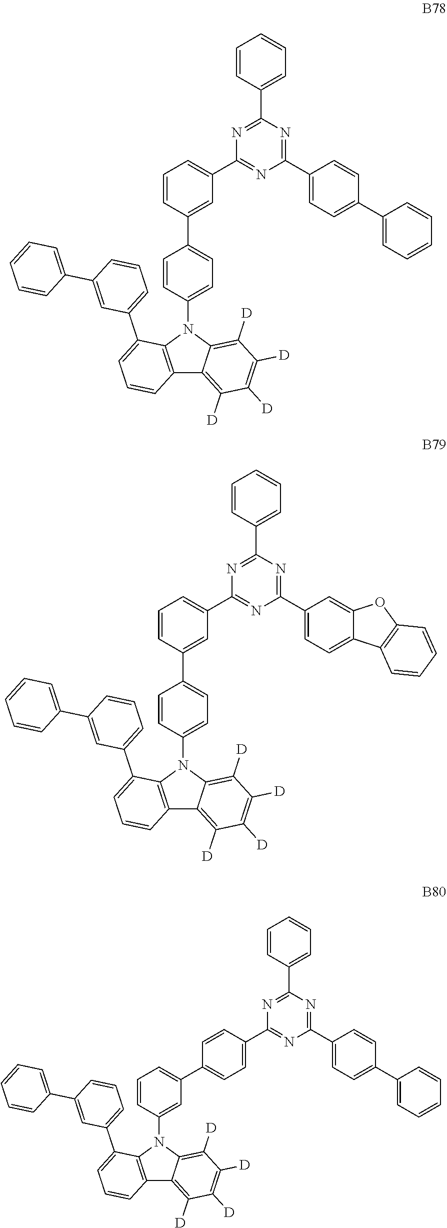 [SMILES: [2H]c1c([2H])c([2H])c2c(c1[2H])c1cccc(-c3cccc(-c4ccccc4)c3)c1n2-c1ccc(-c2cccc(-c3nc(-c4ccccc4)nc(-c4ccc(-c5ccccc5)cc4)n3)c2)cc1 ; [2H]c1c([2H])c([2H])c2c(c1[2H])c1cccc(-c3cccc(-c4ccccc4)c3)c1n2-c1ccc(-c2cccc(-c3nc(-c4ccccc4)nc(-c4ccc5c(c4)oc4ccccc45)n3)c2)cc1 ; [2H]c1c([2H])c([2H])c2c(c1[2H])c1cccc(-c3cccc(-c4ccccc4)c3)c1n2-c1cccc(-c2ccc(-c3nc(-c4ccccc4)nc(-c4ccc(-c5ccccc5)cc4)n3)cc2)c1]

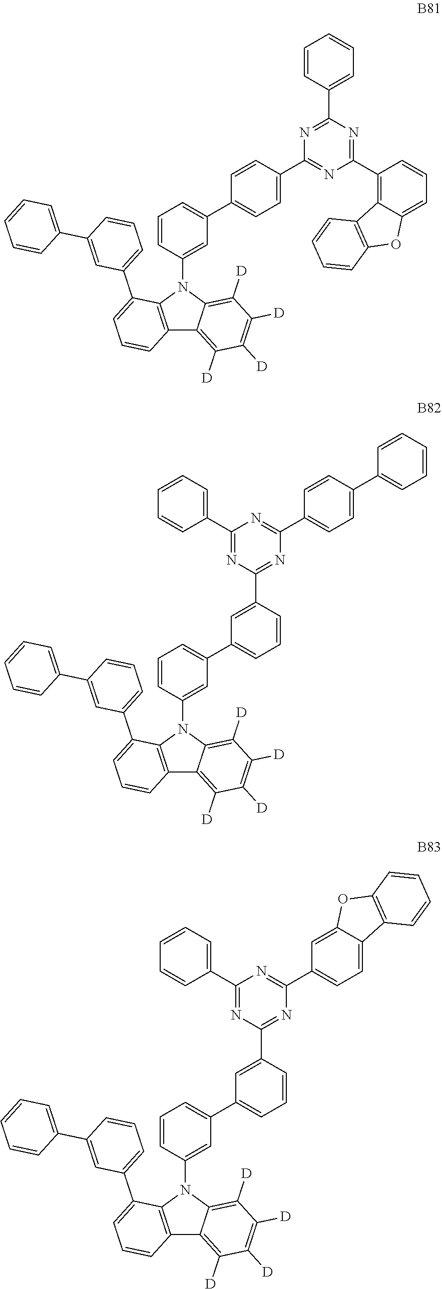 [SMILES: [2H]c1c([2H])c([2H])c2c(c1[2H])c1cccc(-c3cccc(-c4ccccc4)c3)c1n2-c1cccc(-c2ccc(-c3nc(-c4ccccc4)nc(-c4cccc5oc6ccccc6c45)n3)cc2)c1 ; [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccc5c(c4)oc4ccccc45)n3)c2)c1]

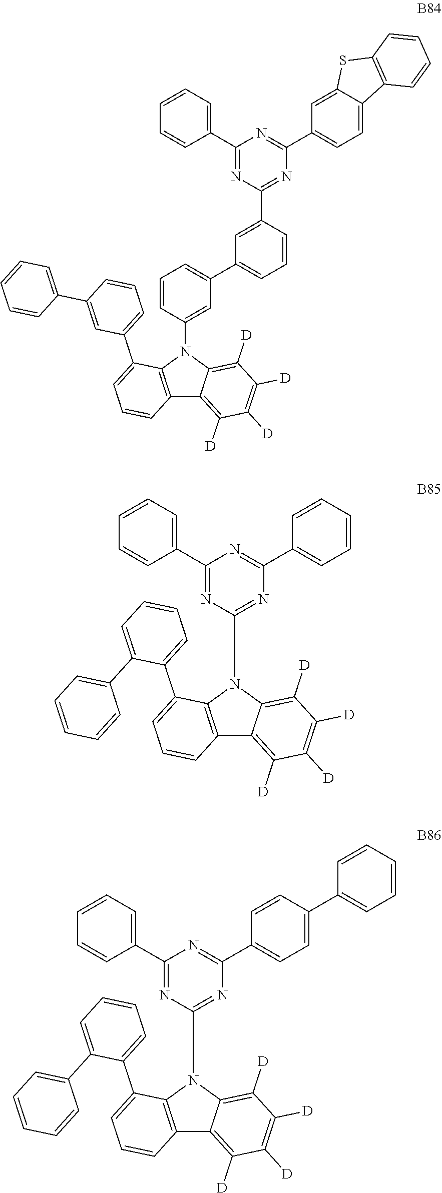 [SMILES: [2H]c1c([2H])c([2H])c2c(c1[2H])c1cccc(-c3cccc(-c4ccccc4)c3)c1n2-c1cccc(-c2cccc(-c3nc(-c4ccccc4)nc(-c4ccc5c(c4)sc4ccccc45)n3)c2)c1 ; [2H]c1c([2H])c([2H])c2c(c1[2H])c1cccc(-c3ccccc3-c3ccccc3)c1n2-c1nc(-c2ccccc2)nc(-c2ccccc2)n1 ; [2H]c1c([2H])c([2H])c2c(c1[2H])c1cccc(-c3ccccc3-c3ccccc3)c1n2-c1nc(-c2ccccc2)nc(-c2ccc(-c3ccccc3)cc2)n1]

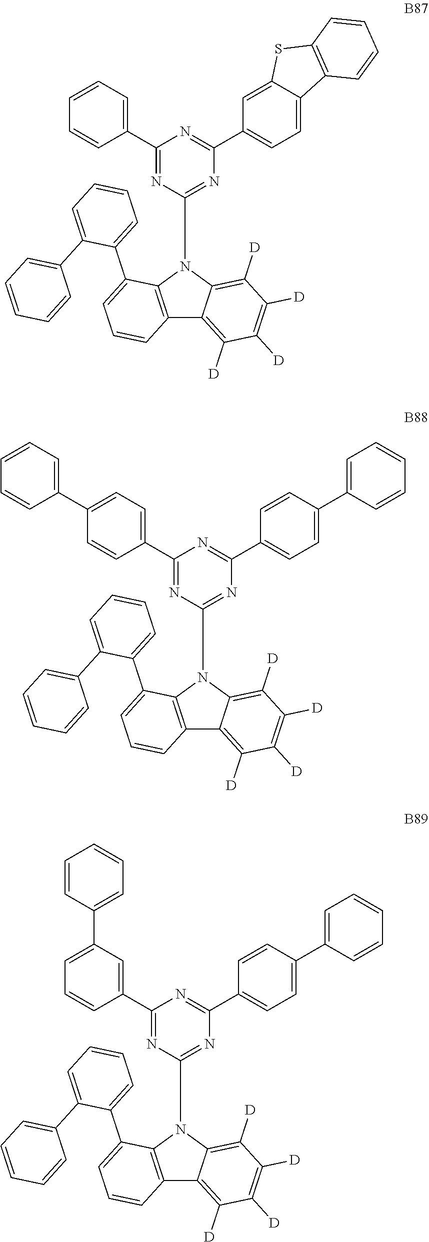 [SMILES: [2H]c1c([2H])c([2H])c2c(c1[2H])c1cccc(-c3ccccc3-c3ccccc3)c1n2-c1nc(-c2ccccc2)nc(-c2ccc3c(c2)sc2ccccc23)n1 ; [2H]c1c([2H])c([2H])c2c(c1[2H])c1cccc(-c3ccccc3-c3ccccc3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3ccccc3-c3ccccc3)c1n2-c1nc(-c2ccc(-c3ccccc3)cc2)nc(-c2cccc(-c3ccccc3)c2)n1]

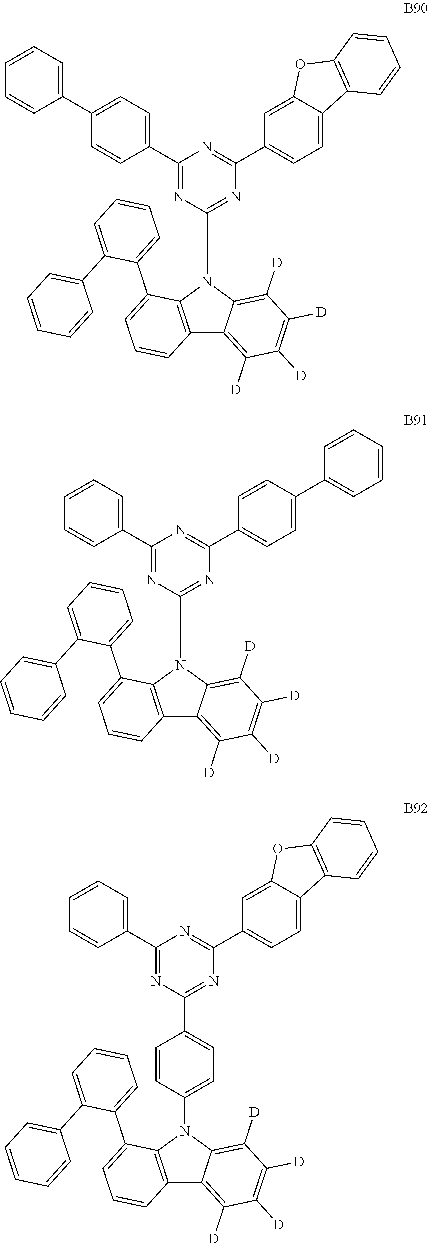 [SMILES: [2H]c1c([2H])c([2H])c2c(c1[2H])c1cccc(-c3ccccc3-c3ccccc3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3ccccc3-c3ccccc3)c1n2-c1nc(-c2ccccc2)nc(-c2ccc(-c3ccccc3)cc2)n1 ; [2H]c1c([2H])c([2H])c2c(c1[2H])c1cccc(-c3ccccc3-c3ccccc3)c1n2-c1ccc(-c2nc(-c3ccccc3)nc(-c3ccc4c(c3)oc3ccccc34)n2)cc1]

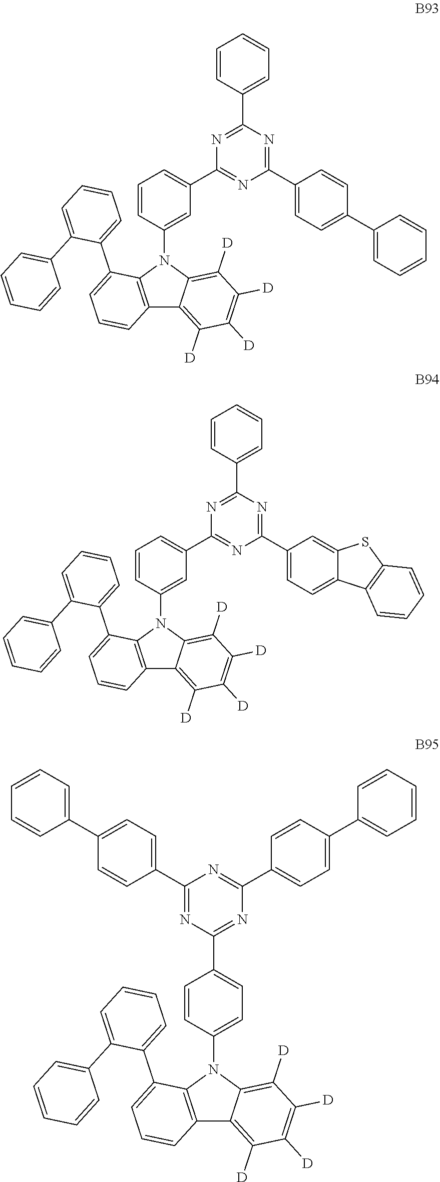 [SMILES: [2H]c1c([2H])c([2H])c2c(c1[2H])c1cccc(-c3ccccc3-c3ccccc3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3ccccc3-c3ccccc3)c1n2-c1cccc(-c2nc(-c3ccccc3)nc(-c3ccc4c(c3)sc3ccccc34)n2)c1 ; [2H]c1c([2H])c([2H])c2c(c1[2H])c1cccc(-c3ccccc3-c3ccccc3)c1n2-c1ccc(-c2nc(-c3ccc(-c4ccccc4)cc3)nc(-c3ccc(-c4ccccc4)cc3)n2)cc1]

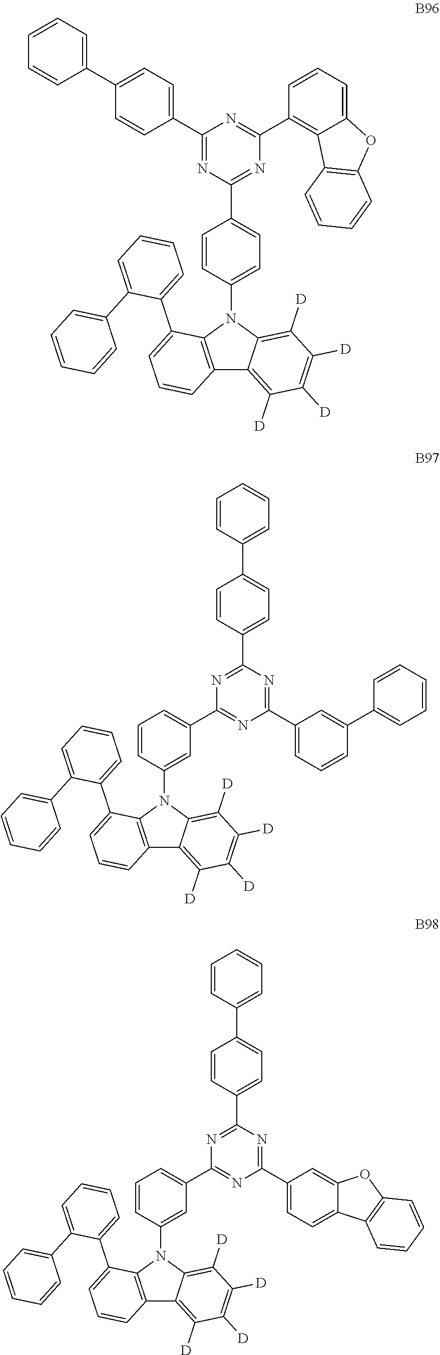 [SMILES: [2H]c1c([2H])c([2H])c2c(c1[2H])c1cccc(-c3ccccc3-c3ccccc3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3ccccc3-c3ccccc3)c1n2-c1cccc(-c2nc(-c3ccc(-c4ccccc4)cc3)nc(-c3cccc(-c4ccccc4)c3)n2)c1 ; [2H]c1c([2H])c([2H])c2c(c1[2H])c1cccc(-c3ccccc3-c3ccccc3)c1n2-c1cccc(-c2nc(-c3ccc(-c4ccccc4)cc3)nc(-c3ccc4c(c3)oc3ccccc34)n2)c1]

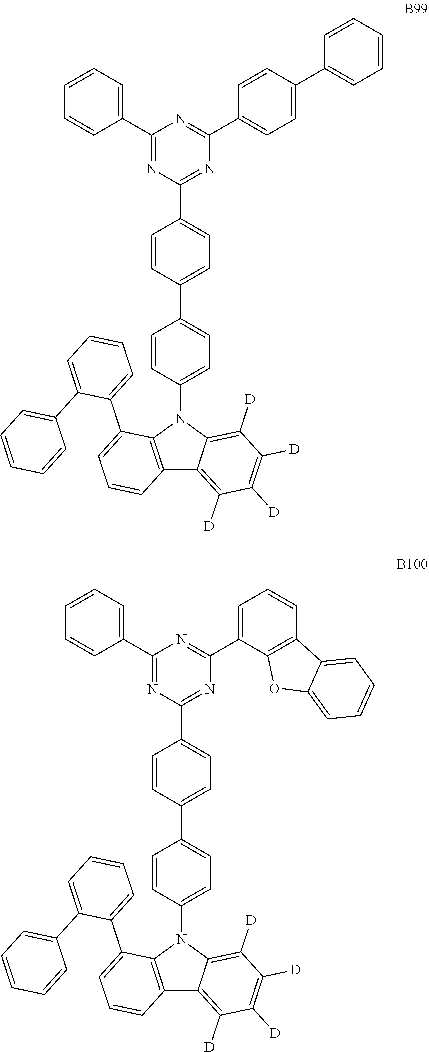 [SMILES: [2H]c1c([2H])c([2H])c2c(c1[2H])c1cccc(-c3ccccc3-c3ccccc3)c1n2-c1ccc(-c2ccc(-c3nc(-c4ccccc4)nc(-c4ccc(-c5ccccc5)cc4)n3)cc2)cc1 ; [2H]c1c([2H])c([2H])c2c(c1[2H])c1cccc(-c3ccccc3-c3ccccc3)c1n2-c1ccc(-c2ccc(-c3nc(-c4ccccc4)nc(-c4cccc5c4oc4ccccc45)n3)cc2)cc1]

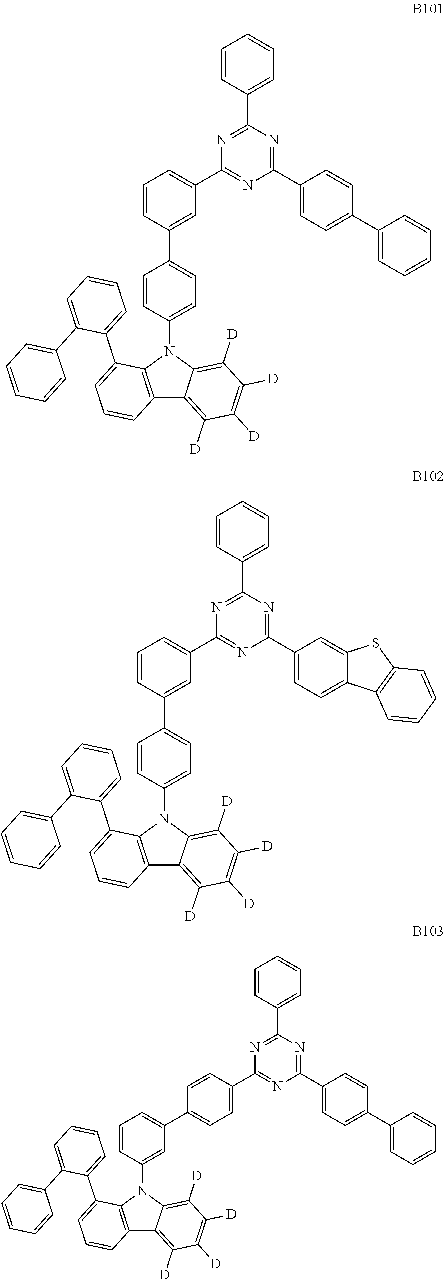 [SMILES: [2H]c1c([2H])c([2H])c2c(c1[2H])c1cccc(-c3ccccc3-c3ccccc3)c1n2-c1ccc(-c2cccc(-c3nc(-c4ccccc4)nc(-c4ccc(-c5ccccc5)cc4)n3)c2)cc1 ; [2H]c1c([2H])c([2H])c2c(c1[2H])c1cccc(-c3ccccc3-c3ccccc3)c1n2-c1ccc(-c2cccc(-c3nc(-c4ccccc4)nc(-c4ccc5c(c4)sc4ccccc45)n3)c2)cc1 ; [2H]c1c([2H])c([2H])c2c(c1[2H])c1cccc(-c3ccccc3-c3ccccc3)c1n2-c1cccc(-c2ccc(-c3nc(-c4ccccc4)nc(-c4ccc(-c5ccccc5)cc4)n3)cc2)c1]

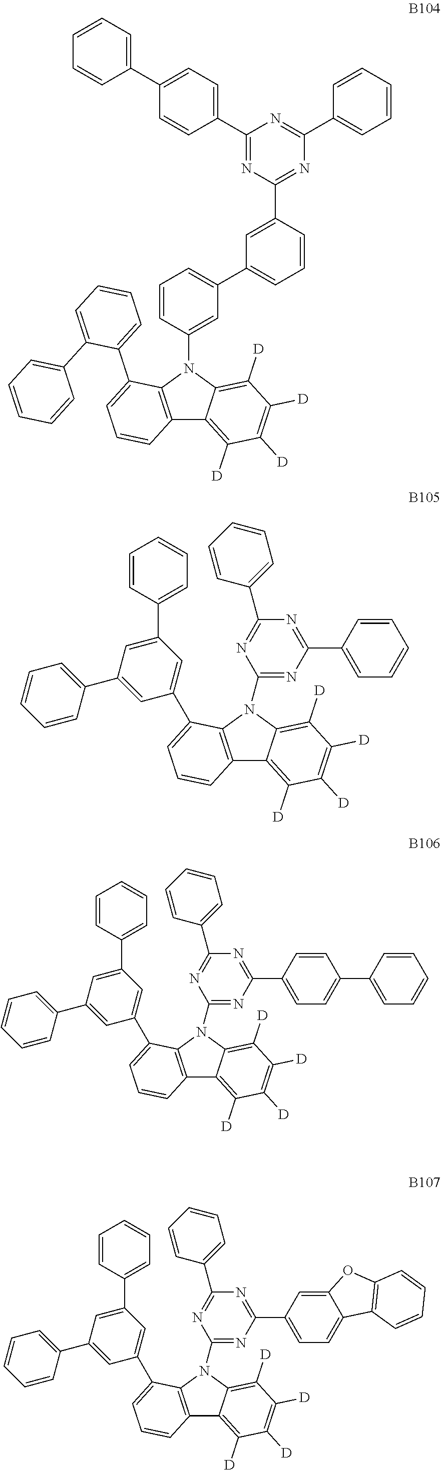 [SMILES: [2H]c1c([2H])c([2H])c2c(c1[2H])c1cccc(-c3ccccc3-c3ccccc3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccccc2)n1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc(-c3ccccc3)cc2)n1 ; [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc3c(c2)oc2ccccc23)n1]

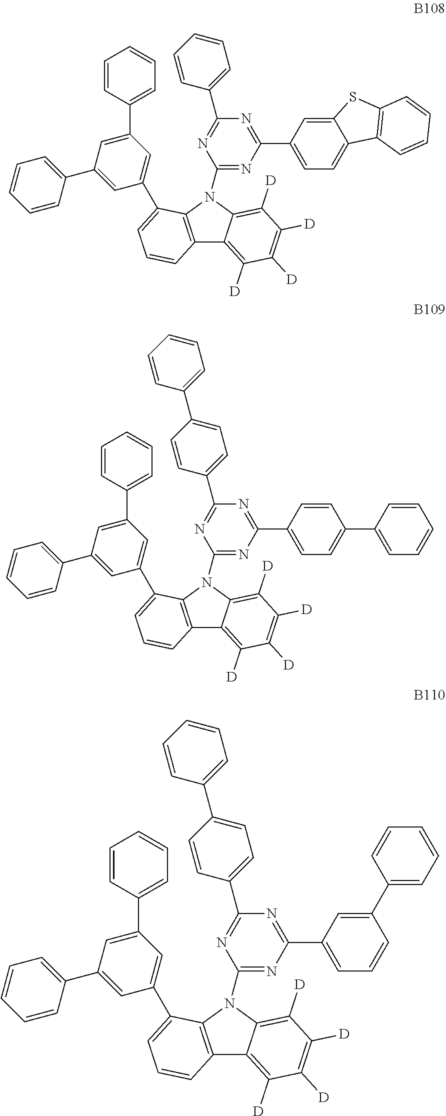 [SMILES: [2H]c1c([2H])c([2H])c2c(c1[2H])c1cccc(-c3cc(-c4ccccc4)cc(-c4ccccc4)c3)c1n2-c1nc(-c2ccccc2)nc(-c2ccc3c(c2)sc2ccccc23)n1 ; [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2ccc(-c3ccccc3)cc2)n1 ; [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2cccc(-c3ccccc3)c2)n1]

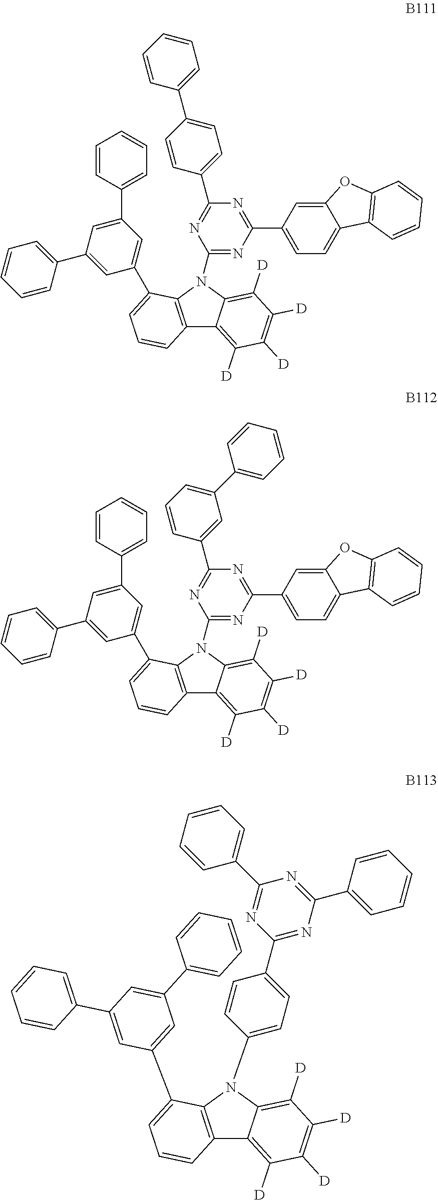 [SMILES: [2H]c1c([2H])c([2H])c2c(c1[2H])c1cccc(-c3cc(-c4ccccc4)cc(-c4ccccc4)c3)c1n2-c1nc(-c2ccc(-c3ccccc3)cc2)nc(-c2ccc3c(c2)oc2ccccc23)n1 ; [2H]c1c([2H])c([2H])c2c(c1[2H])c1cccc(-c3cc(-c4ccccc4)cc(-c4ccccc4)c3)c1n2-c1nc(-c2cccc(-c3ccccc3)c2)nc(-c2ccc3c(c2)oc2ccccc23)n1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3ccccc3)n2)cc1]

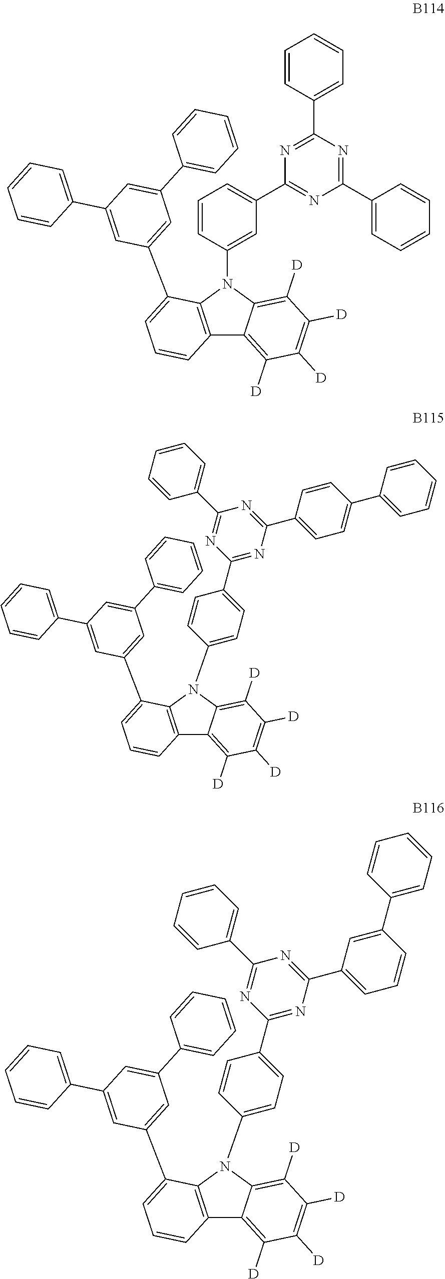 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccccc3)nc(-c3ccccc3)n2)c1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3ccc(-c4ccccc4)cc3)n2)cc1 ; [2H]c1c([2H])c([2H])c2c(c1[2H])c1cccc(-c3cc(-c4ccccc4)cc(-c4ccccc4)c3)c1n2-c1ccc(-c2nc(-c3ccccc3)nc(-c3cccc(-c4ccccc4)c3)n2)cc1]

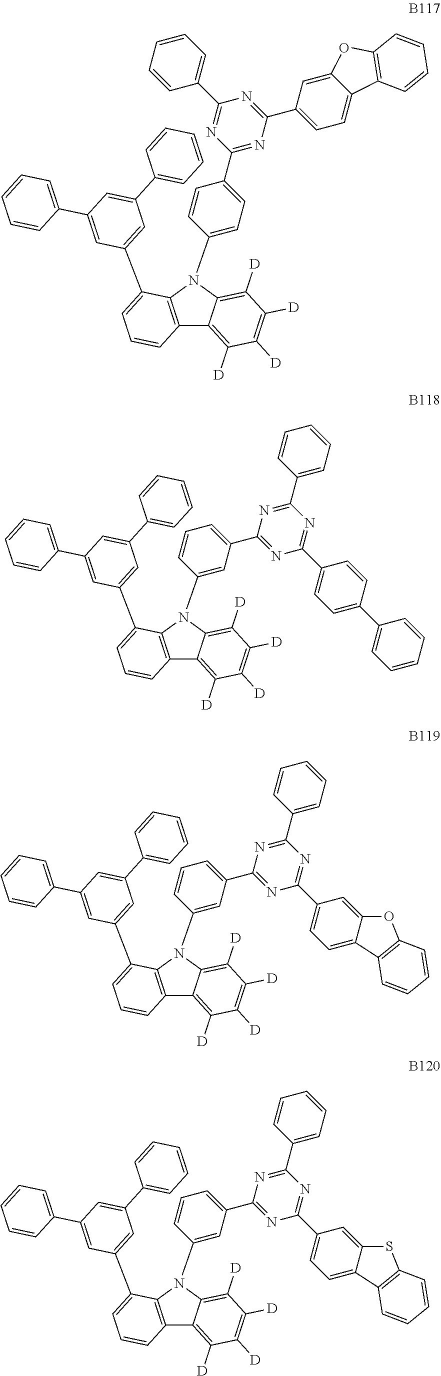

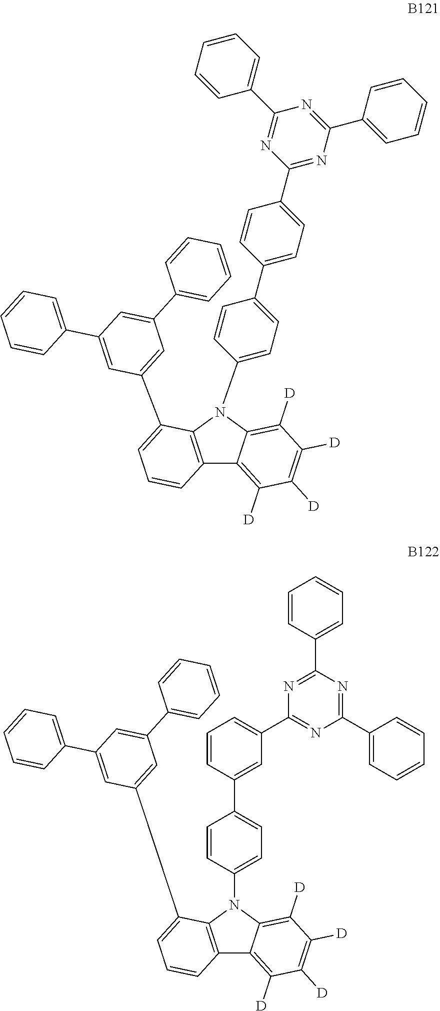 [SMILES: [2H]c1c([2H])c([2H])c2c(c1[2H])c1cccc(-c3cc(-c4ccccc4)cc(-c4ccccc4)c3)c1n2-c1ccc(-c2ccc(-c3nc(-c4ccccc4)nc(-c4ccccc4)n3)cc2)cc1 ; [2H]c1c([2H])c([2H])c2c(c1[2H])c1cccc(-c3cc(-c4ccccc4)cc(-c4ccccc4)c3)c1n2-c1ccc(-c2cccc(-c3nc(-c4ccccc4)nc(-c4ccccc4)n3)c2)cc1]

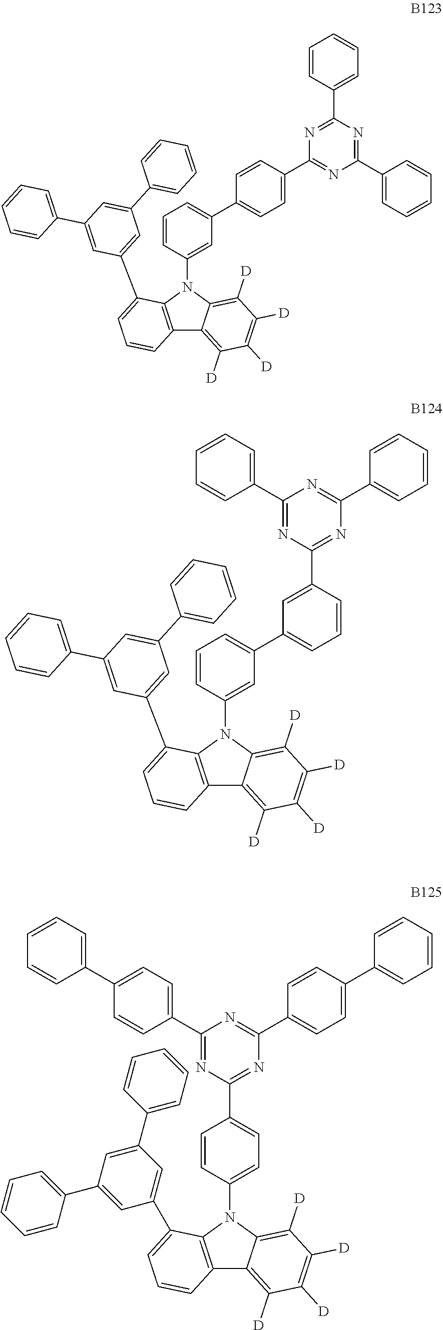 [SMILES: [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccccc4)n3)cc2)c1 ; [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccccc4)n3)c2)c1 ; [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3ccc(-c4ccccc4)cc3)n2)cc1]

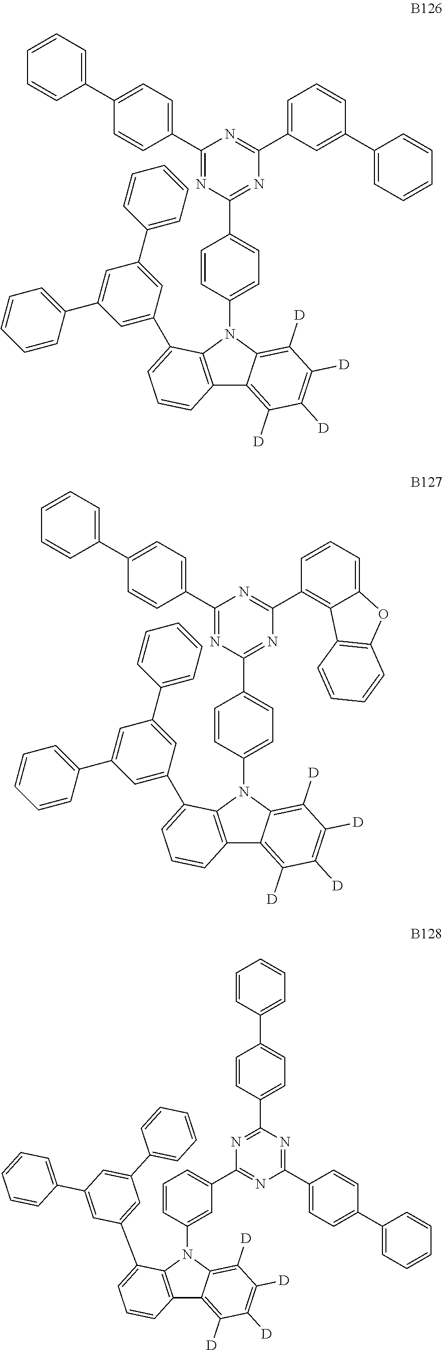 [SMILES: [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3cccc(-c4ccccc4)c3)n2)cc1 ; [2H]c1c([2H])c([2H])c2c(c1[2H])c1cccc(-c3cc(-c4ccccc4)cc(-c4ccccc4)c3)c1n2-c1ccc(-c2nc(-c3ccc(-c4ccccc4)cc3)nc(-c3cccc4oc5ccccc5c34)n2)cc1 ; [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3ccc(-c4ccccc4)cc3)n2)c1]

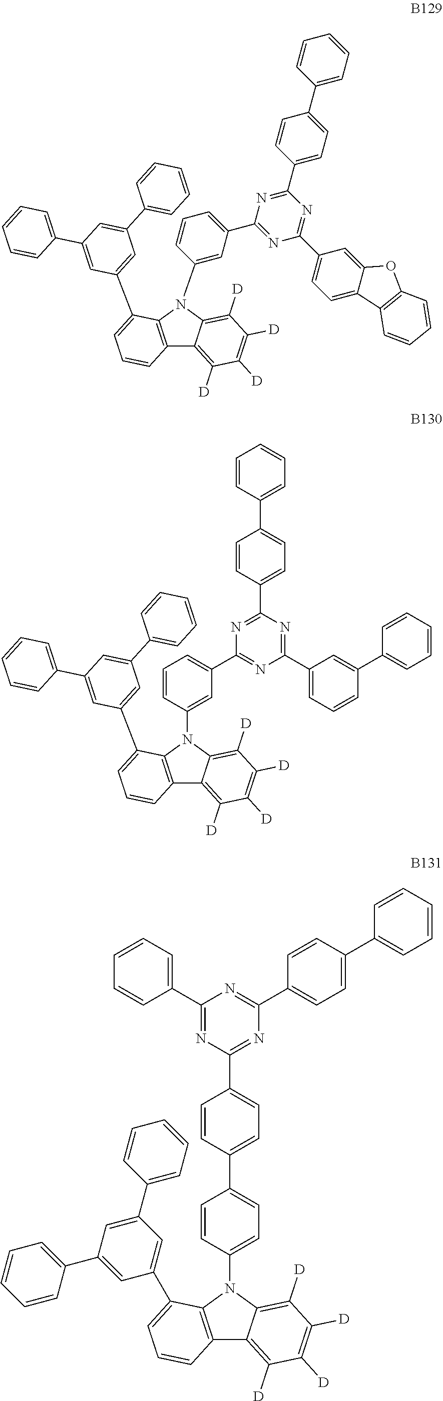 [SMILES: [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3ccc4c(c3)oc3ccccc34)n2)c1 ; [2H]c1c([2H])c([2H])c2c(c1[2H])c1cccc(-c3cc(-c4ccccc4)cc(-c4ccccc4)c3)c1n2-c1cccc(-c2nc(-c3ccc(-c4ccccc4)cc3)nc(-c3cccc(-c4ccccc4)c3)n2)c1 ; [2H]c1c([2H])c([2H])c2c(c1[2H])c1cccc(-c3cc(-c4ccccc4)cc(-c4ccccc4)c3)c1n2-c1ccc(-c2ccc(-c3nc(-c4ccccc4)nc(-c4ccc(-c5ccccc5)cc4)n3)cc2)cc1]

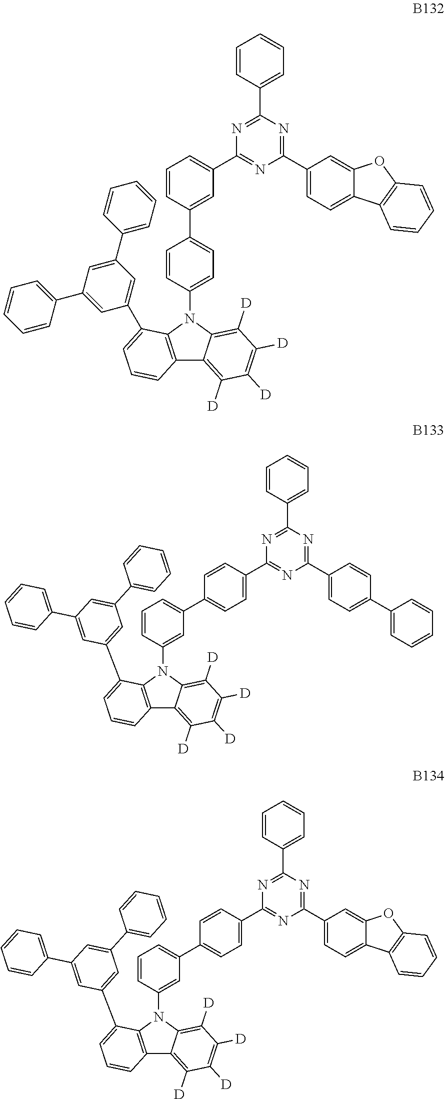 [SMILES: [2H]c1c([2H])c([2H])c2c(c1[2H])c1cccc(-c3cc(-c4ccccc4)cc(-c4ccccc4)c3)c1n2-c1ccc(-c2cccc(-c3nc(-c4ccccc4)nc(-c4ccc5c(c4)oc4ccccc45)n3)c2)cc1 ; [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccc(-c5ccccc5)cc4)n3)cc2)c1 ; [2H]c1c([2H])c([2H])c2c(c1[2H])c1cccc(-c3cc(-c4ccccc4)cc(-c4ccccc4)c3)c1n2-c1cccc(-c2ccc(-c3nc(-c4ccccc4)nc(-c4ccc5c(c4)oc4ccccc45)n3)cc2)c1]

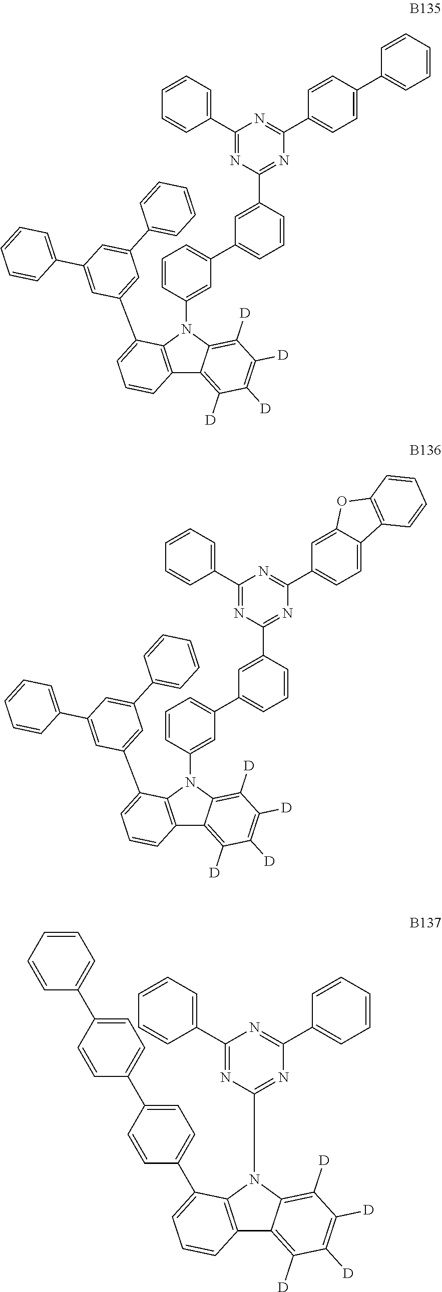 [SMILES: [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccc(-c5ccccc5)cc4)n3)c2)c1 ; [2H]c1c([2H])c([2H])c2c(c1[2H])c1cccc(-c3cc(-c4ccccc4)cc(-c4ccccc4)c3)c1n2-c1cccc(-c2cccc(-c3nc(-c4ccccc4)nc(-c4ccc5c(c4)oc4ccccc45)n3)c2)c1 ; [2H]c1c([2H])c([2H])c2c(c1[2H])c1cccc(-c3ccc(-c4ccc(-c5ccccc5)cc4)cc3)c1n2-c1nc(-c2ccccc2)nc(-c2ccccc2)n1]

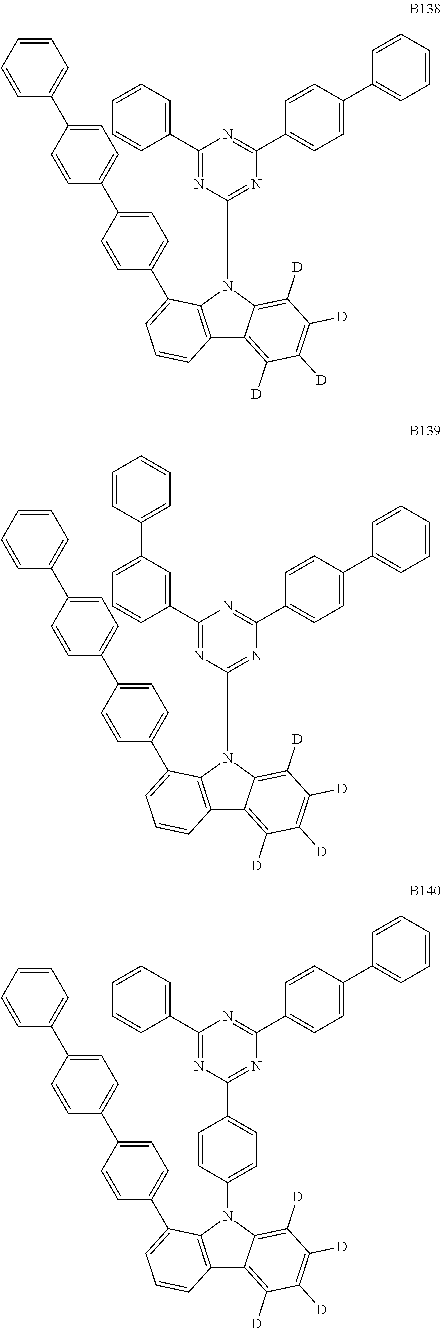 [SMILES: [2H]c1c([2H])c([2H])c2c(c1[2H])c1cccc(-c3ccc(-c4ccc(-c5ccccc5)cc4)cc3)c1n2-c1nc(-c2ccccc2)nc(-c2ccc(-c3ccccc3)cc2)n1 ; [2H]c1c([2H])c([2H])c2c(c1[2H])c1cccc(-c3ccc(-c4ccc(-c5ccccc5)cc4)cc3)c1n2-c1nc(-c2ccc(-c3ccccc3)cc2)nc(-c2cccc(-c3ccccc3)c2)n1 ; [2H]c1c([2H])c([2H])c2c(c1[2H])c1cccc(-c3ccc(-c4ccc(-c5ccccc5)cc4)cc3)c1n2-c1ccc(-c2nc(-c3ccccc3)nc(-c3ccc(-c4ccccc4)cc3)n2)cc1]

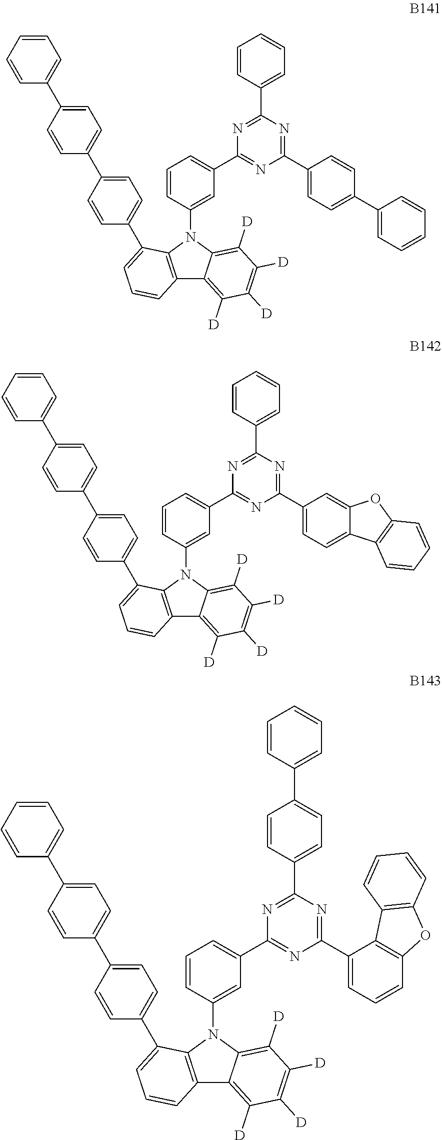 [SMILES: [2H]c1c([2H])c([2H])c2c(c1[2H])c1cccc(-c3ccc(-c4ccc(-c5ccccc5)cc4)cc3)c1n2-c1cccc(-c2nc(-c3ccccc3)nc(-c3ccc(-c4ccccc4)cc3)n2)c1 ; [2H]c1c([2H])c([2H])c2c(c1[2H])c1cccc(-c3ccc(-c4ccc(-c5ccccc5)cc4)cc3)c1n2-c1cccc(-c2nc(-c3ccccc3)nc(-c3ccc4c(c3)oc3ccccc34)n2)c1 ; [2H]c1c([2H])c([2H])c2c(c1[2H])c1cccc(-c3ccc(-c4ccc(-c5ccccc5)cc4)cc3)c1n2-c1cccc(-c2nc(-c3ccc(-c4ccccc4)cc3)nc(-c3cccc4oc5ccccc5c34)n2)c1]

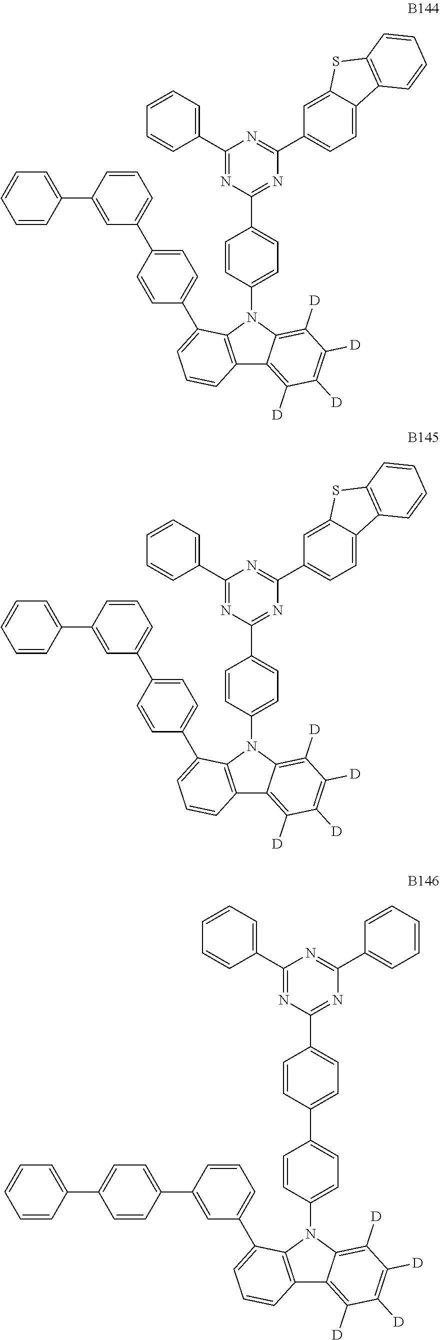 [SMILES: [2H]c1c([2H])c([2H])c2c(c1[2H])c1cccc(-c3ccc(-c4cccc(-c5ccccc5)c4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3ccc(-c4cccc(-c5ccccc5)c4)cc3)c1n2-c1ccc(-c2nc(-c3ccccc3)nc(-c3ccc4c(c3)sc3ccccc34)n2)cc1 ; [2H]c1c([2H])c([2H])c2c(c1[2H])c1cccc(-c3cccc(-c4ccc(-c5ccccc5)cc4)c3)c1n2-c1ccc(-c2ccc(-c3nc(-c4ccccc4)nc(-c4ccccc4)n3)cc2)cc1]

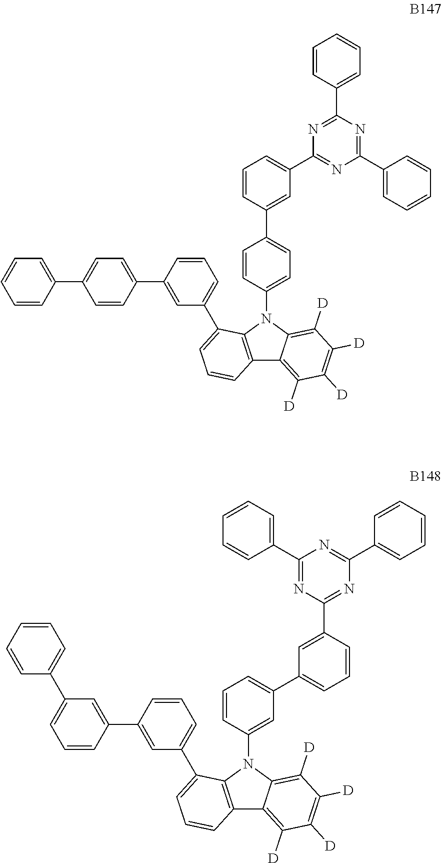 [SMILES: [2H]c1c([2H])c([2H])c2c(c1[2H])c1cccc(-c3cccc(-c4ccc(-c5ccccc5)cc4)c3)c1n2-c1ccc(-c2cccc(-c3nc(-c4ccccc4)nc(-c4ccccc4)n3)c2)cc1 ; [2H]c1c([2H])c([2H])c2c(c1[2H])c1cccc(-c3cccc(-c4cccc(-c5ccccc5)c4)c3)c1n2-c1cccc(-c2cccc(-c3nc(-c4ccccc4)nc(-c4ccccc4)n3)c2)c1]

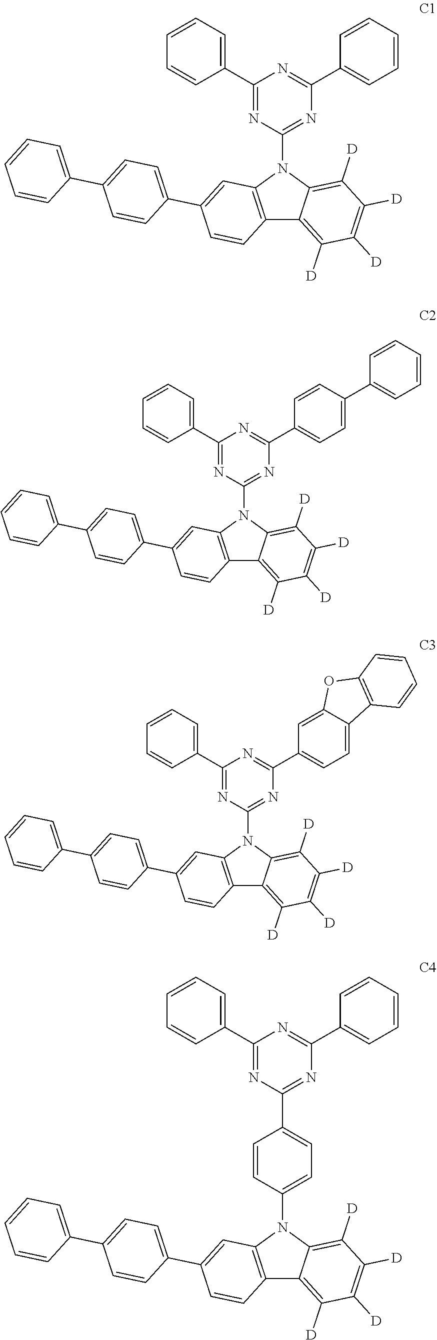 [SMILES: [2H]c1c([2H])c([2H])c2c(c1[2H])c1ccc(-c3ccc(-c4ccccc4)cc3)cc1n2-c1nc(-c2ccccc2)nc(-c2ccccc2)n1 ; [2H]c1c([2H])c([2H])c2c(c1[2H])c1ccc(-c3ccc(-c4ccccc4)cc3)cc1n2-c1nc(-c2ccccc2)nc(-c2ccc(-c3ccccc3)cc2)n1 ; [2H]c1c([2H])c([2H])c2c(c1[2H])c1ccc(-c3ccc(-c4ccccc4)cc3)cc1n2-c1nc(-c2ccccc2)nc(-c2ccc3c(c2)oc2ccccc23)n1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccccc3)n2)cc1]

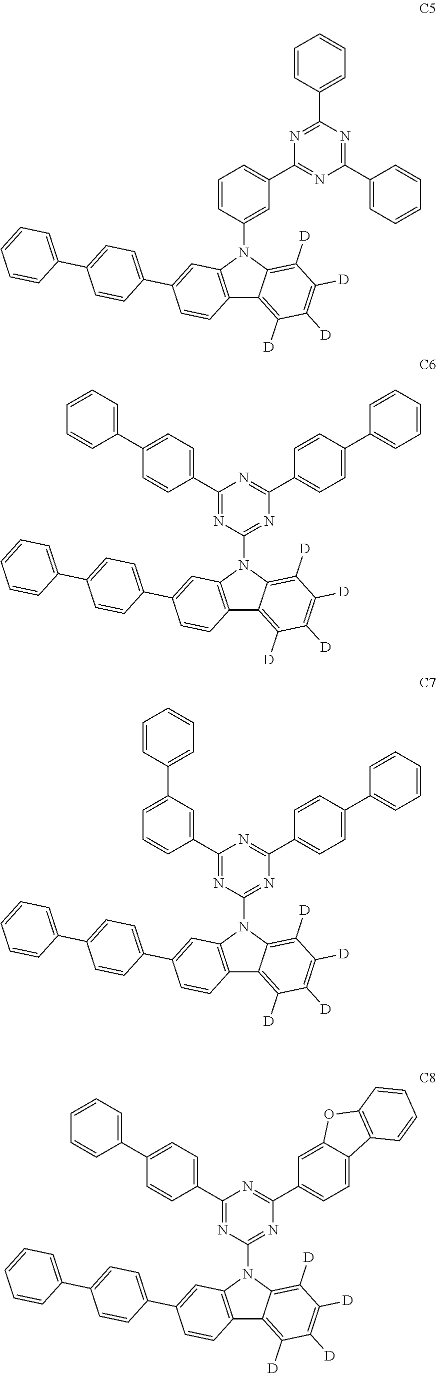 [SMILES: [2H]c1c([2H])c([2H])c2c(c1[2H])c1ccc(-c3ccc(-c4ccccc4)cc3)cc1n2-c1cccc(-c2nc(-c3ccccc3)nc(-c3ccccc3)n2)c1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2ccc(-c3ccccc3)cc2)n1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2cccc(-c3ccccc3)c2)n1 ; [2H]c1c([2H])c([2H])c2c(c1[2H])c1ccc(-c3ccc(-c4ccccc4)cc3)cc1n2-c1nc(-c2ccc(-c3ccccc3)cc2)nc(-c2ccc3c(c2)oc2ccccc23)n1]

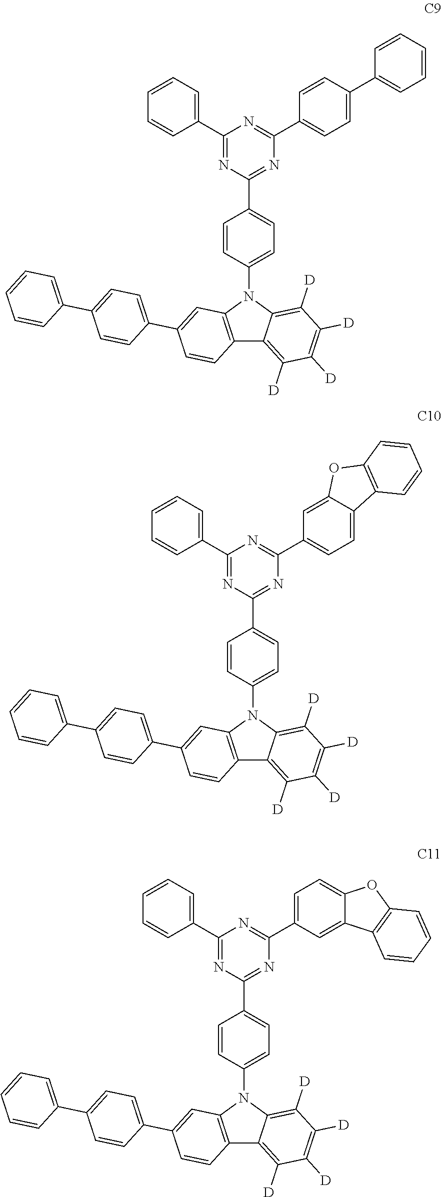 [SMILES: [2H]c1c([2H])c([2H])c2c(c1[2H])c1ccc(-c3ccc(-c4ccccc4)cc3)cc1n2-c1ccc(-c2nc(-c3ccccc3)nc(-c3ccc(-c4ccccc4)cc3)n2)cc1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccc4c(c3)oc3ccccc34)n2)cc1 ; [2H]c1c([2H])c([2H])c2c(c1[2H])c1ccc(-c3ccc(-c4ccccc4)cc3)cc1n2-c1ccc(-c2nc(-c3ccccc3)nc(-c3ccc4oc5ccccc5c4c3)n2)cc1]

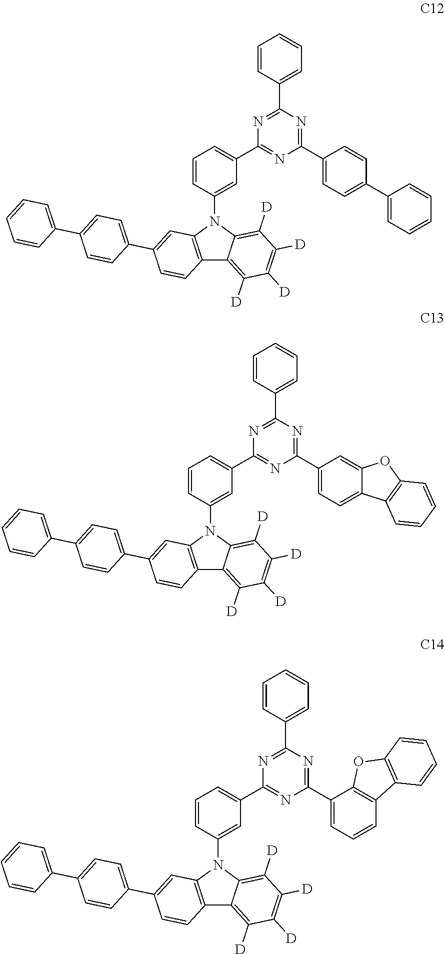 [SMILES: [2H]c1c([2H])c([2H])c2c(c1[2H])c1ccc(-c3ccc(-c4ccccc4)cc3)cc1n2-c1cccc(-c2nc(-c3ccccc3)nc(-c3ccc(-c4ccccc4)cc3)n2)c1 ; [2H]c1c([2H])c([2H])c2c(c1[2H])c1ccc(-c3ccc(-c4ccccc4)cc3)cc1n2-c1cccc(-c2nc(-c3ccccc3)nc(-c3ccc4c(c3)oc3ccccc34)n2)c1 ; [2H]c1c([2H])c([2H])c2c(c1[2H])c1ccc(-c3ccc(-c4ccccc4)cc3)cc1n2-c1cccc(-c2nc(-c3ccccc3)nc(-c3cccc4c3oc3ccccc34)n2)c1]

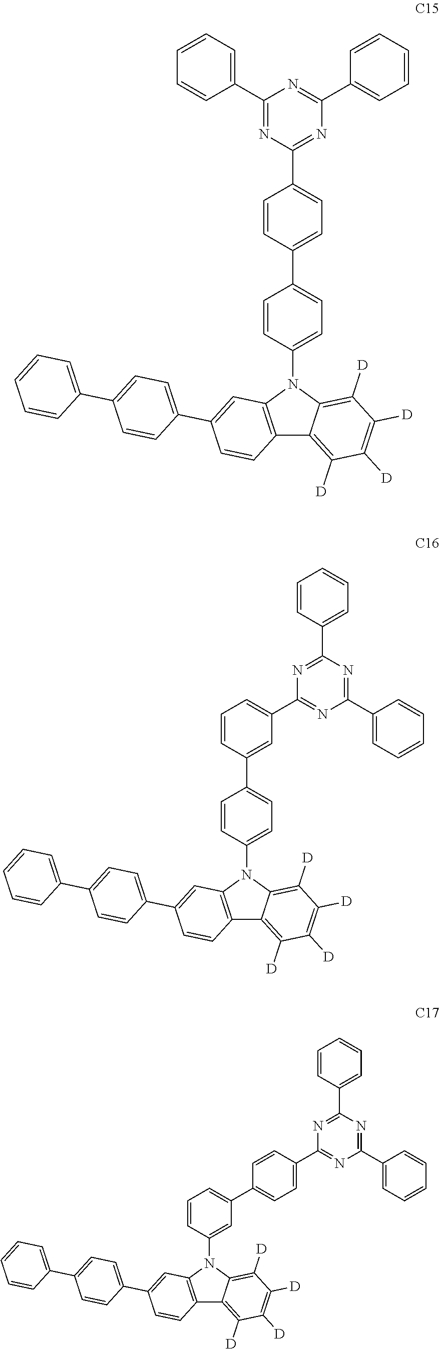 [SMILES: [2H]c1c([2H])c([2H])c2c(c1[2H])c1ccc(-c3ccc(-c4ccccc4)cc3)cc1n2-c1ccc(-c2ccc(-c3nc(-c4ccccc4)nc(-c4ccccc4)n3)cc2)cc1 ; [2H]c1c([2H])c([2H])c2c(c1[2H])c1ccc(-c3ccc(-c4ccccc4)cc3)cc1n2-c1ccc(-c2cccc(-c3nc(-c4ccccc4)nc(-c4ccccc4)n3)c2)cc1 ; [2H]c1c([2H])c([2H])c2c(c1[2H])c1ccc(-c3ccc(-c4ccccc4)cc3)cc1n2-c1cccc(-c2ccc(-c3nc(-c4ccccc4)nc(-c4ccccc4)n3)cc2)c1]

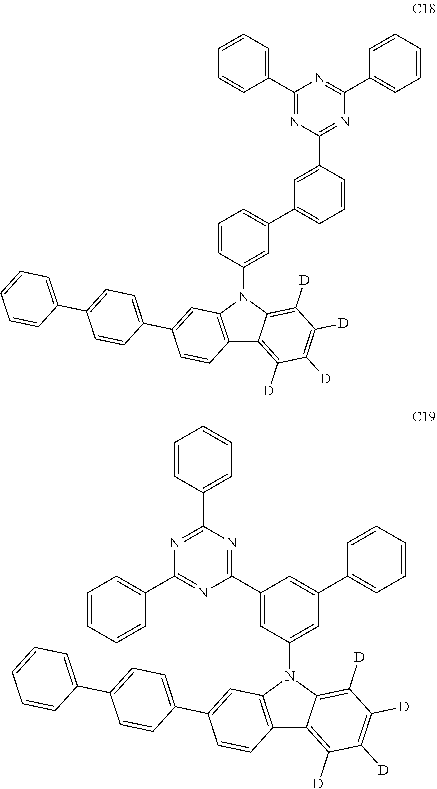 [SMILES: [2H]c1c([2H])c([2H])c2c(c1[2H])c1ccc(-c3ccc(-c4ccccc4)cc3)cc1n2-c1cccc(-c2cccc(-c3nc(-c4ccccc4)nc(-c4ccccc4)n3)c2)c1 ; [2H]c1c([2H])c([2H])c2c(c1[2H])c1ccc(-c3ccc(-c4ccccc4)cc3)cc1n2-c1cc(-c2ccccc2)cc(-c2nc(-c3ccccc3)nc(-c3ccccc3)n2)c1]

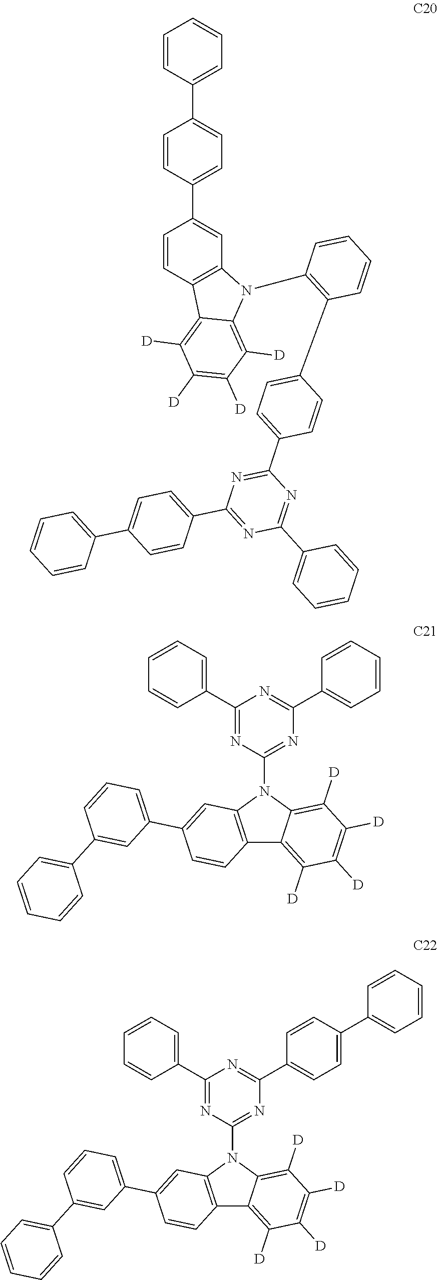

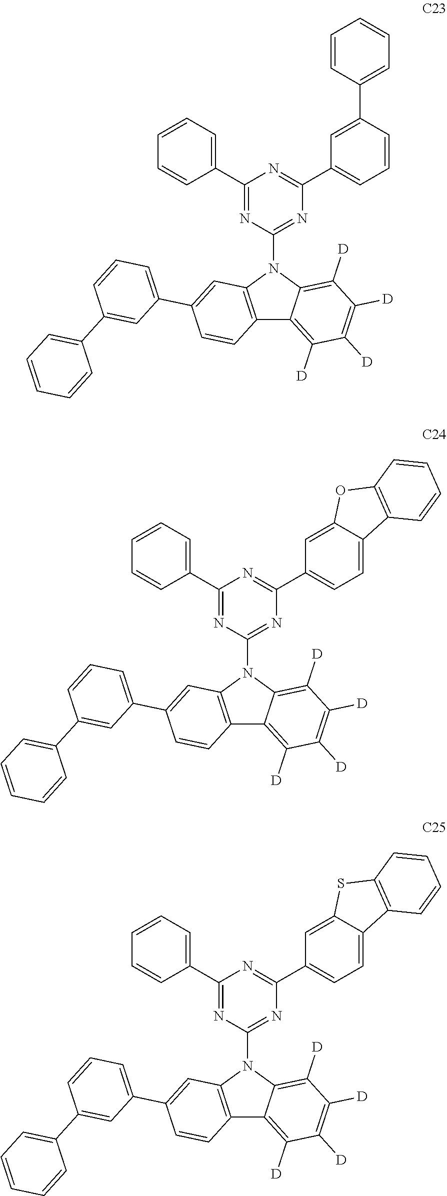 [SMILES: [2H]c1c([2H])c([2H])c2c(c1[2H])c1ccc(-c3cccc(-c4ccccc4)c3)cc1n2-c1nc(-c2ccccc2)nc(-c2cccc(-c3ccccc3)c2)n1 ; [2H]c1c([2H])c([2H])c2c(c1[2H])c1ccc(-c3cccc(-c4ccccc4)c3)cc1n2-c1nc(-c2ccccc2)nc(-c2ccc3c(c2)oc2ccccc23)n1 ; [2H]c1c([2H])c([2H])c2c(c1[2H])c1ccc(-c3cccc(-c4ccccc4)c3)cc1n2-c1nc(-c2ccccc2)nc(-c2ccc3c(c2)sc2ccccc23)n1]

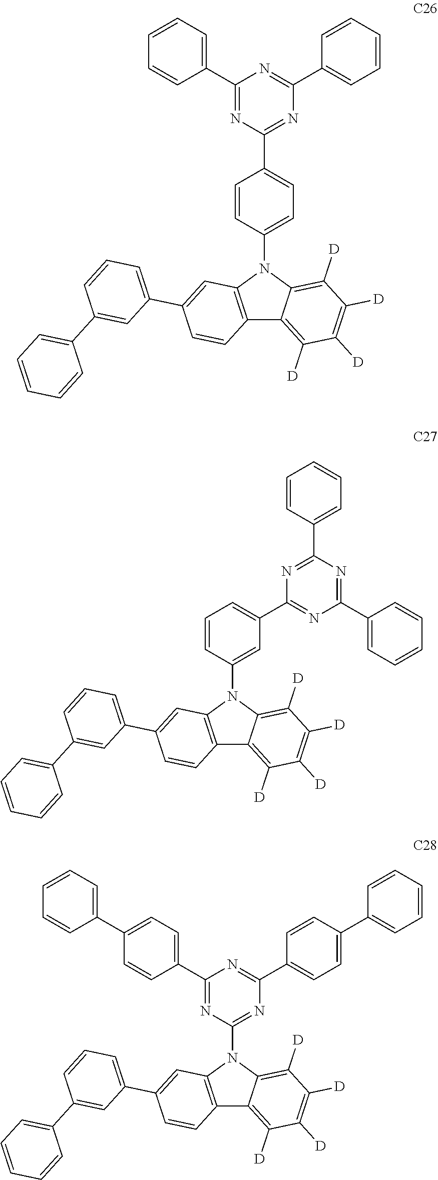 [SMILES: [2H]c1c([2H])c([2H])c2c(c1[2H])c1ccc(-c3cccc(-c4ccccc4)c3)cc1n2-c1ccc(-c2nc(-c3ccccc3)nc(-c3ccccc3)n2)cc1 ; [2H]c1c([2H])c([2H])c2c(c1[2H])c1ccc(-c3cccc(-c4ccccc4)c3)cc1n2-c1cccc(-c2nc(-c3ccccc3)nc(-c3ccccc3)n2)c1 ; [2H]c1c([2H])c([2H])c2c(c1[2H])c1ccc(-c3cccc(-c4ccccc4)c3)cc1n2-c1nc(-c2ccc(-c3ccccc3)cc2)nc(-c2ccc(-c3ccccc3)cc2)n1]

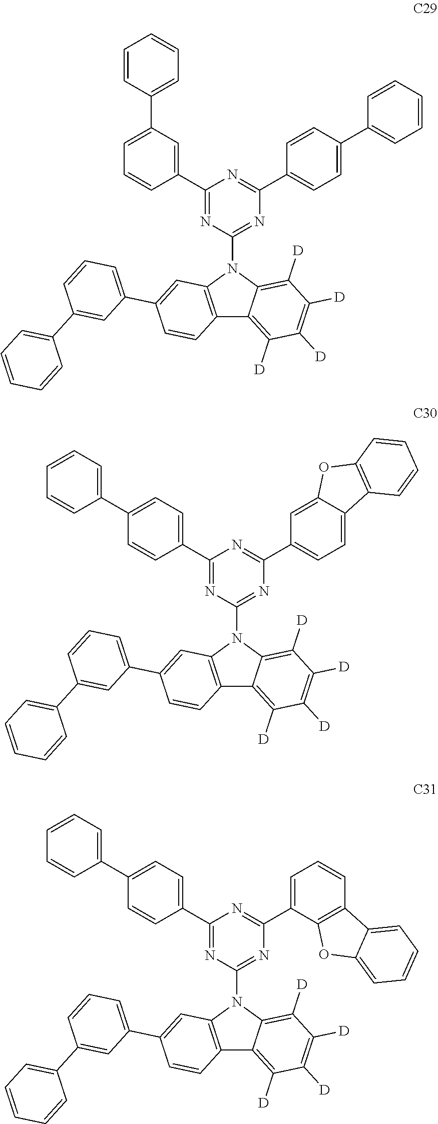 [SMILES: [2H]c1c([2H])c([2H])c2c(c1[2H])c1ccc(-c3cccc(-c4ccccc4)c3)cc1n2-c1nc(-c2ccc(-c3ccccc3)cc2)nc(-c2cccc(-c3ccccc3)c2)n1 ; [2H]c1c([2H])c([2H])c2c(c1[2H])c1ccc(-c3cccc(-c4ccccc4)c3)cc1n2-c1nc(-c2ccc(-c3ccccc3)cc2)nc(-c2ccc3c(c2)oc2ccccc23)n1 ; [2H]c1c([2H])c([2H])c2c(c1[2H])c1ccc(-c3cccc(-c4ccccc4)c3)cc1n2-c1nc(-c2ccc(-c3ccccc3)cc2)nc(-c2cccc3c2oc2ccccc23)n1]

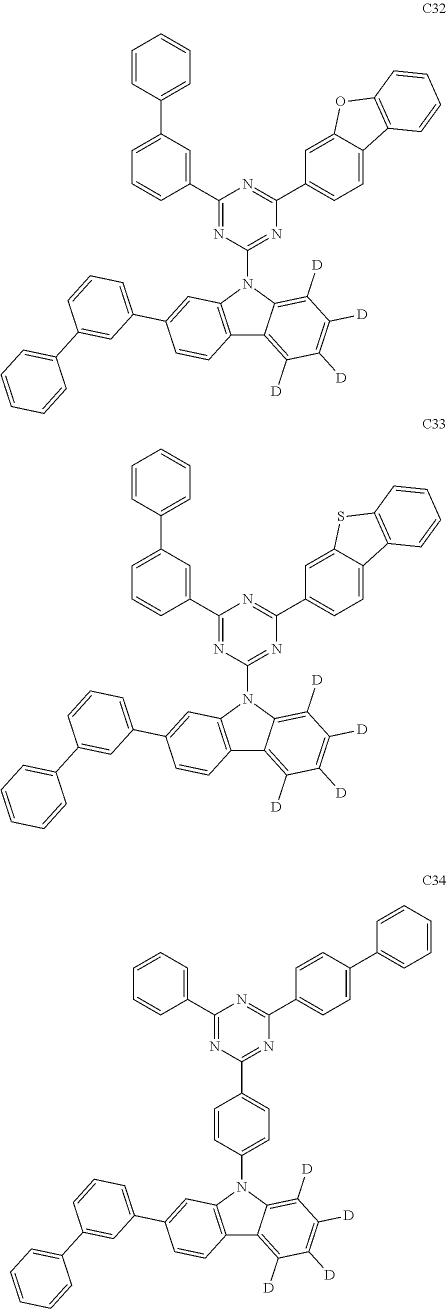 [SMILES: [2H]c1c([2H])c([2H])c2c(c1[2H])c1ccc(-c3cccc(-c4ccccc4)c3)cc1n2-c1nc(-c2cccc(-c3ccccc3)c2)nc(-c2ccc3c(c2)oc2ccccc23)n1 ; [2H]c1c([2H])c([2H])c2c(c1[2H])c1ccc(-c3cccc(-c4ccccc4)c3)cc1n2-c1nc(-c2cccc(-c3ccccc3)c2)nc(-c2ccc3c(c2)sc2ccccc23)n1 ; [2H]c1c([2H])c([2H])c2c(c1[2H])c1ccc(-c3cccc(-c4ccccc4)c3)cc1n2-c1ccc(-c2nc(-c3ccccc3)nc(-c3ccc(-c4ccccc4)cc3)n2)cc1]

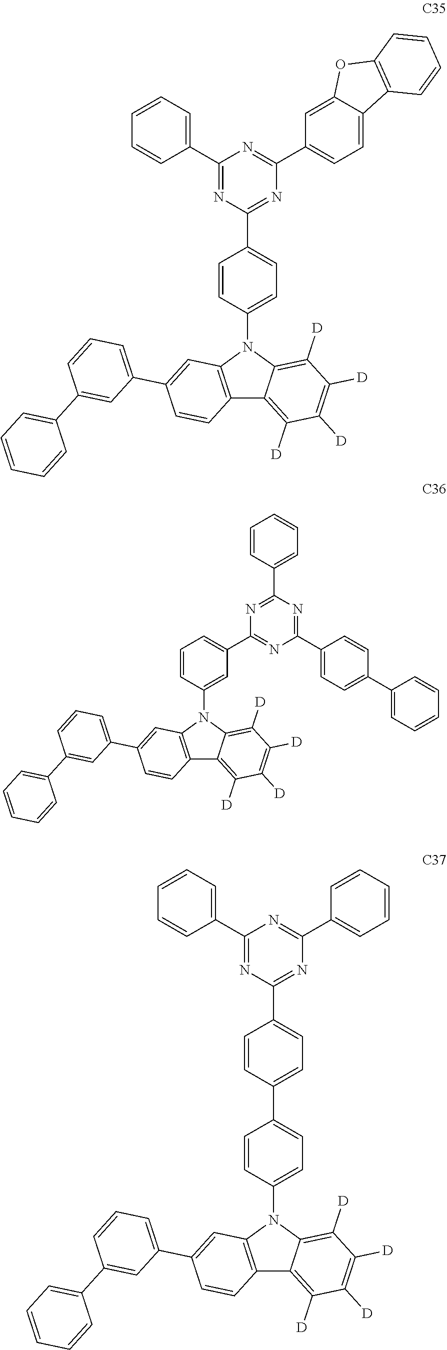 [SMILES: [2H]c1c([2H])c([2H])c2c(c1[2H])c1ccc(-c3cccc(-c4ccccc4)c3)cc1n2-c1ccc(-c2nc(-c3ccccc3)nc(-c3ccc4c(c3)oc3ccccc34)n2)cc1 ; [2H]c1c([2H])c([2H])c2c(c1[2H])c1ccc(-c3cccc(-c4ccccc4)c3)cc1n2-c1cccc(-c2nc(-c3ccccc3)nc(-c3ccc(-c4ccccc4)cc3)n2)c1 ; [2H]c1c([2H])c([2H])c2c(c1[2H])c1ccc(-c3cccc(-c4ccccc4)c3)cc1n2-c1ccc(-c2ccc(-c3nc(-c4ccccc4)nc(-c4ccccc4)n3)cc2)cc1]

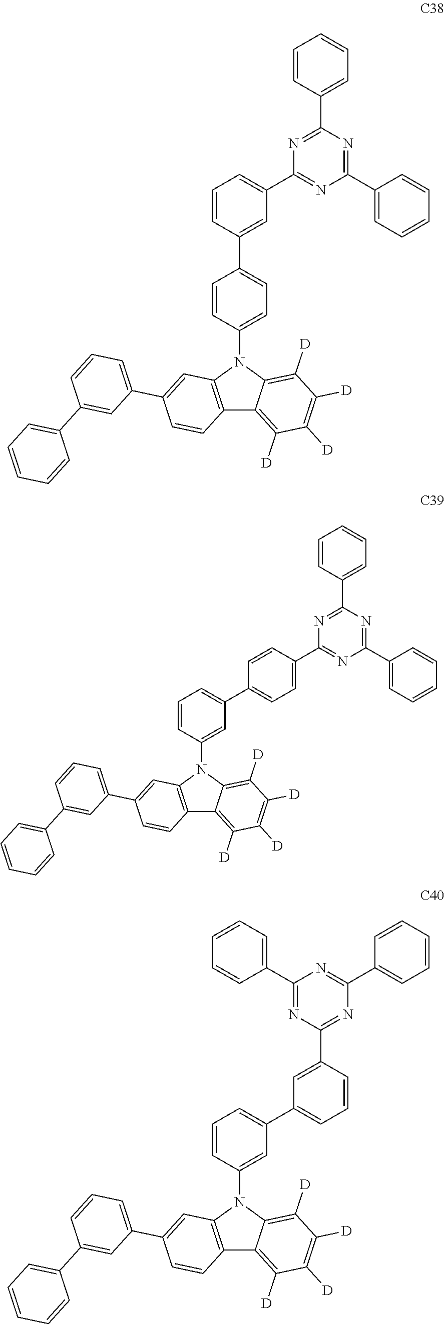 [SMILES: [2H]c1c([2H])c([2H])c2c(c1[2H])c1ccc(-c3cccc(-c4ccccc4)c3)cc1n2-c1ccc(-c2cccc(-c3nc(-c4ccccc4)nc(-c4ccccc4)n3)c2)cc1 ; [2H]c1c([2H])c([2H])c2c(c1[2H])c1ccc(-c3cccc(-c4ccccc4)c3)cc1n2-c1cccc(-c2ccc(-c3nc(-c4ccccc4)nc(-c4ccccc4)n3)cc2)c1 ; [2H]c1c([2H])c([2H])c2c(c1[2H])c1ccc(-c3cccc(-c4ccccc4)c3)cc1n2-c1cccc(-c2cccc(-c3nc(-c4ccccc4)nc(-c4ccccc4)n3)c2)c1]

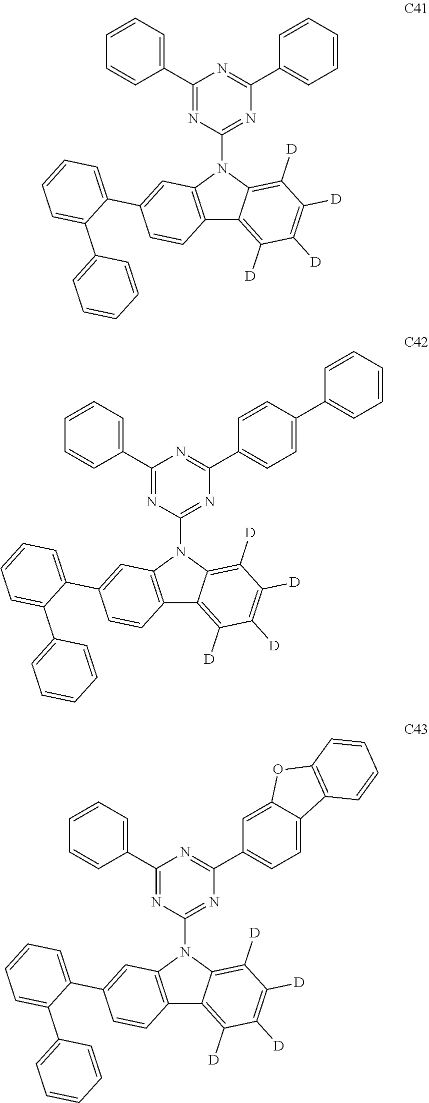 [SMILES: [2H]c1c([2H])c([2H])c2c(c1[2H])c1ccc(-c3ccccc3-c3ccccc3)cc1n2-c1nc(-c2ccccc2)nc(-c2ccccc2)n1 ; [2H]c1c([2H])c([2H])c2c(c1[2H])c1ccc(-c3ccccc3-c3ccccc3)cc1n2-c1nc(-c2ccccc2)nc(-c2ccc(-c3ccccc3)cc2)n1 ; [2H]c1c([2H])c([2H])c2c(c1[2H])c1ccc(-c3ccccc3-c3ccccc3)cc1n2-c1nc(-c2ccccc2)nc(-c2ccc3c(c2)oc2ccccc23)n1]

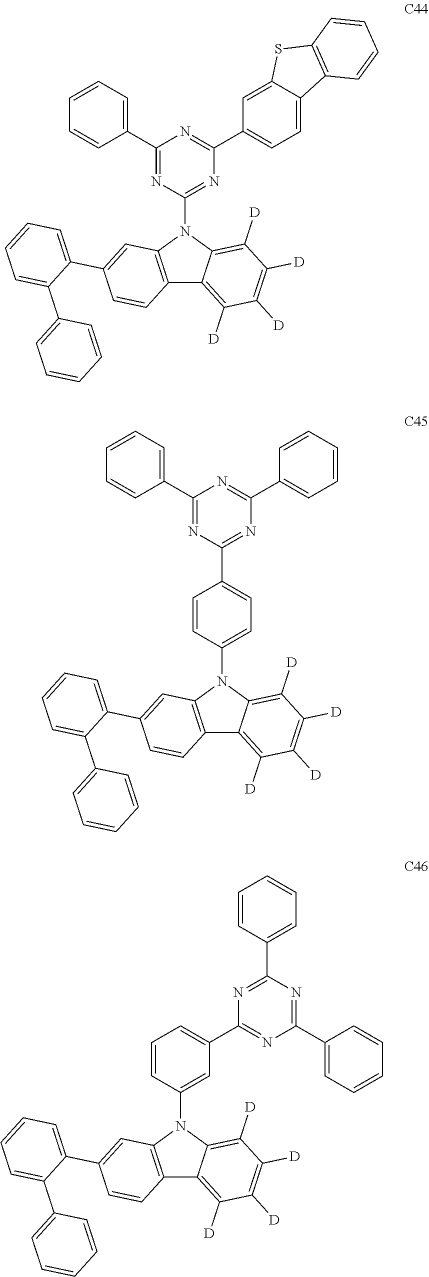 [SMILES: [2H]c1c([2H])c([2H])c2c(c1[2H])c1ccc(-c3ccccc3-c3ccccc3)cc1n2-c1nc(-c2ccccc2)nc(-c2ccc3c(c2)sc2ccccc23)n1 ; [2H]c1c([2H])c([2H])c2c(c1[2H])c1ccc(-c3ccccc3-c3ccccc3)cc1n2-c1ccc(-c2nc(-c3ccccc3)nc(-c3ccccc3)n2)cc1 ; [2H]c1c([2H])c([2H])c2c(c1[2H])c1ccc(-c3ccccc3-c3ccccc3)cc1n2-c1cccc(-c2nc(-c3ccccc3)nc(-c3ccccc3)n2)c1]

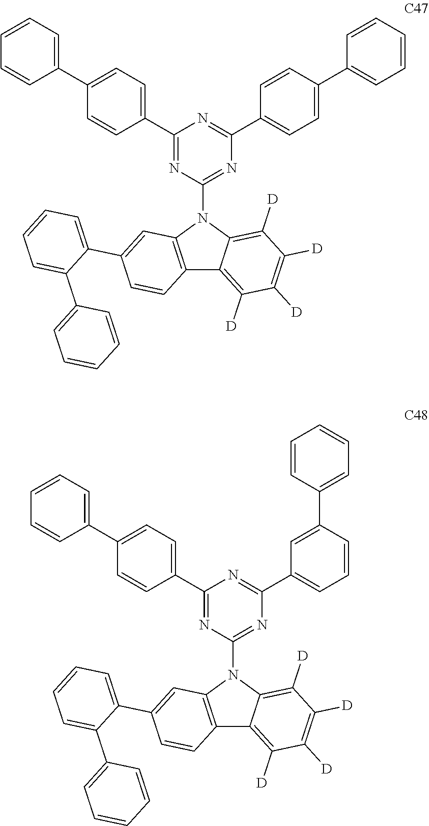 [SMILES: [2H]c1c([2H])c([2H])c2c(c1[2H])c1ccc(-c3ccccc3-c3ccccc3)cc1n2-c1nc(-c2ccc(-c3ccccc3)cc2)nc(-c2ccc(-c3ccccc3)cc2)n1 ; [2H]c1c([2H])c([2H])c2c(c1[2H])c1ccc(-c3ccccc3-c3ccccc3)cc1n2-c1nc(-c2ccc(-c3ccccc3)cc2)nc(-c2cccc(-c3ccccc3)c2)n1]

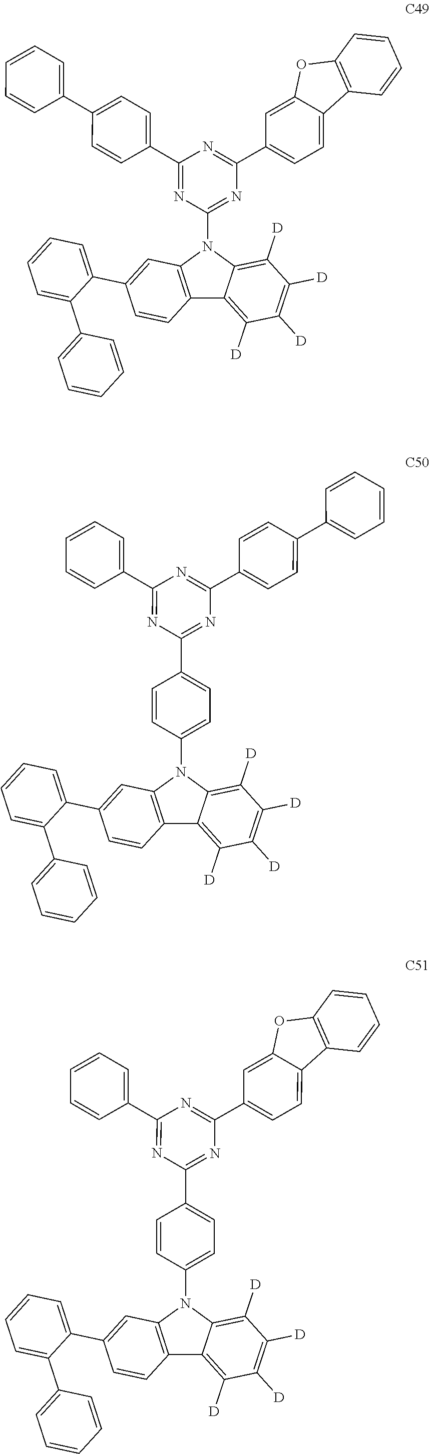 [SMILES: [2H]c1c([2H])c([2H])c2c(c1[2H])c1ccc(-c3ccccc3-c3ccccc3)cc1n2-c1nc(-c2ccc(-c3ccccc3)cc2)nc(-c2ccc3c(c2)oc2ccccc23)n1 ; [2H]c1c([2H])c([2H])c2c(c1[2H])c1ccc(-c3ccccc3-c3ccccc3)cc1n2-c1ccc(-c2nc(-c3ccccc3)nc(-c3ccc(-c4ccccc4)cc3)n2)cc1 ; [2H]c1c([2H])c([2H])c2c(c1[2H])c1ccc(-c3ccccc3-c3ccccc3)cc1n2-c1ccc(-c2nc(-c3ccccc3)nc(-c3ccc4c(c3)oc3ccccc34)n2)cc1]

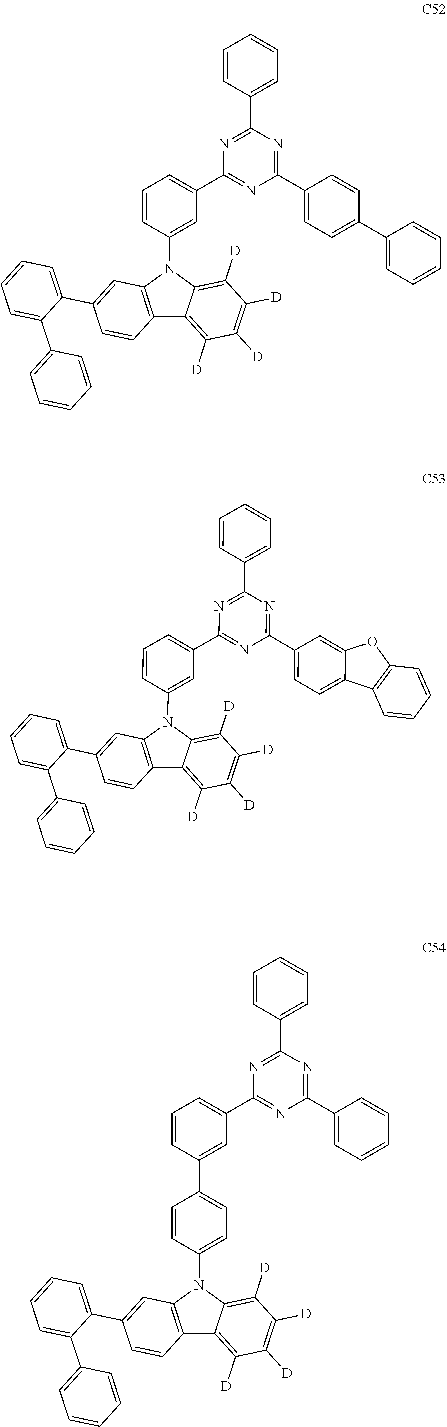 [SMILES: [2H]c1c([2H])c([2H])c2c(c1[2H])c1ccc(-c3ccccc3-c3ccccc3)cc1n2-c1cccc(-c2nc(-c3ccccc3)nc(-c3ccc(-c4ccccc4)cc3)n2)c1 ; [2H]c1c([2H])c([2H])c2c(c1[2H])c1ccc(-c3ccccc3-c3ccccc3)cc1n2-c1cccc(-c2nc(-c3ccccc3)nc(-c3ccc4c(c3)oc3ccccc34)n2)c1 ; [2H]c1c([2H])c([2H])c2c(c1[2H])c1ccc(-c3ccccc3-c3ccccc3)cc1n2-c1ccc(-c2cccc(-c3nc(-c4ccccc4)nc(-c4ccccc4)n3)c2)cc1]

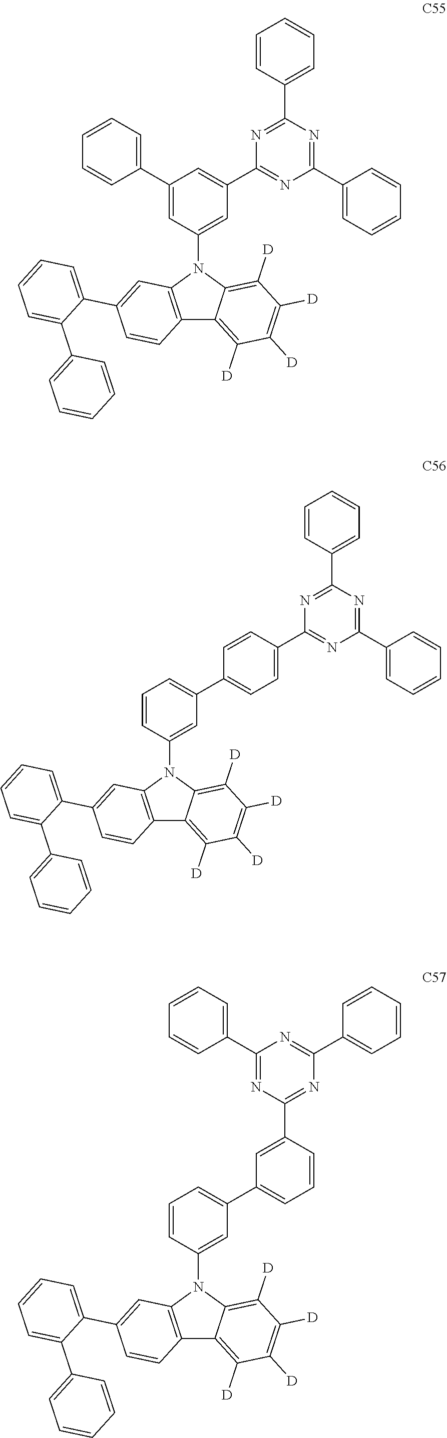 [SMILES: [2H]c1c([2H])c([2H])c2c(c1[2H])c1ccc(-c3ccccc3-c3ccccc3)cc1n2-c1cc(-c2ccccc2)cc(-c2nc(-c3ccccc3)nc(-c3ccccc3)n2)c1 ; [2H]c1c([2H])c([2H])c2c(c1[2H])c1ccc(-c3ccccc3-c3ccccc3)cc1n2-c1cccc(-c2ccc(-c3nc(-c4ccccc4)nc(-c4ccccc4)n3)cc2)c1 ; [2H]c1c([2H])c([2H])c2c(c1[2H])c1ccc(-c3ccccc3-c3ccccc3)cc1n2-c1cccc(-c2cccc(-c3nc(-c4ccccc4)nc(-c4ccccc4)n3)c2)c1]

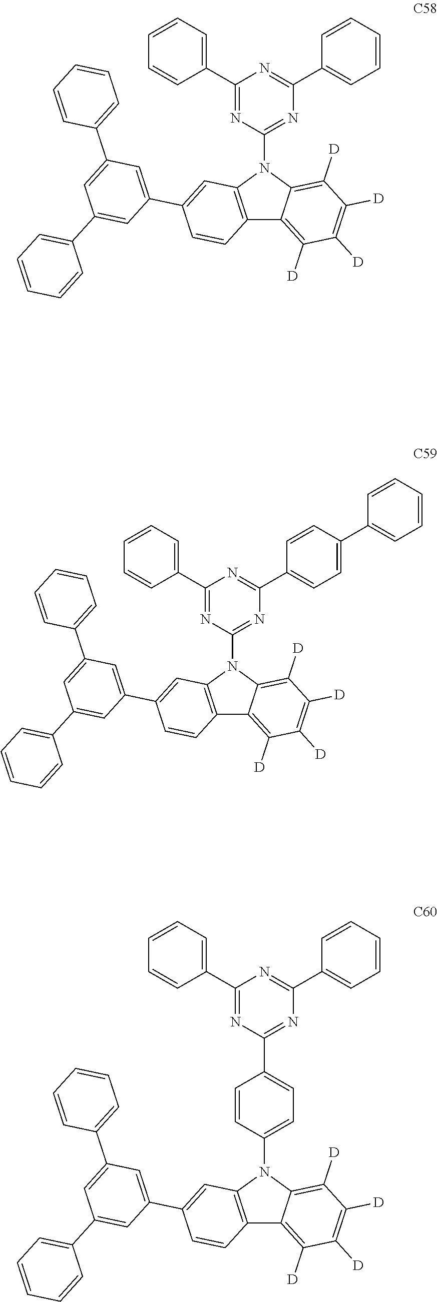 [SMILES: [2H]c1c([2H])c([2H])c2c(c1[2H])c1ccc(-c3cc(-c4ccccc4)cc(-c4ccccc4)c3)cc1n2-c1nc(-c2ccccc2)nc(-c2ccccc2)n1 ; [2H]c1c([2H])c([2H])c2c(c1[2H])c1ccc(-c3cc(-c4ccccc4)cc(-c4ccccc4)c3)cc1n2-c1nc(-c2ccccc2)nc(-c2ccc(-c3ccccc3)cc2)n1 ; [2H]c1c([2H])c([2H])c2c(c1[2H])c1ccc(-c3cc(-c4ccccc4)cc(-c4ccccc4)c3)cc1n2-c1ccc(-c2nc(-c3ccccc3)nc(-c3ccccc3)n2)cc1]

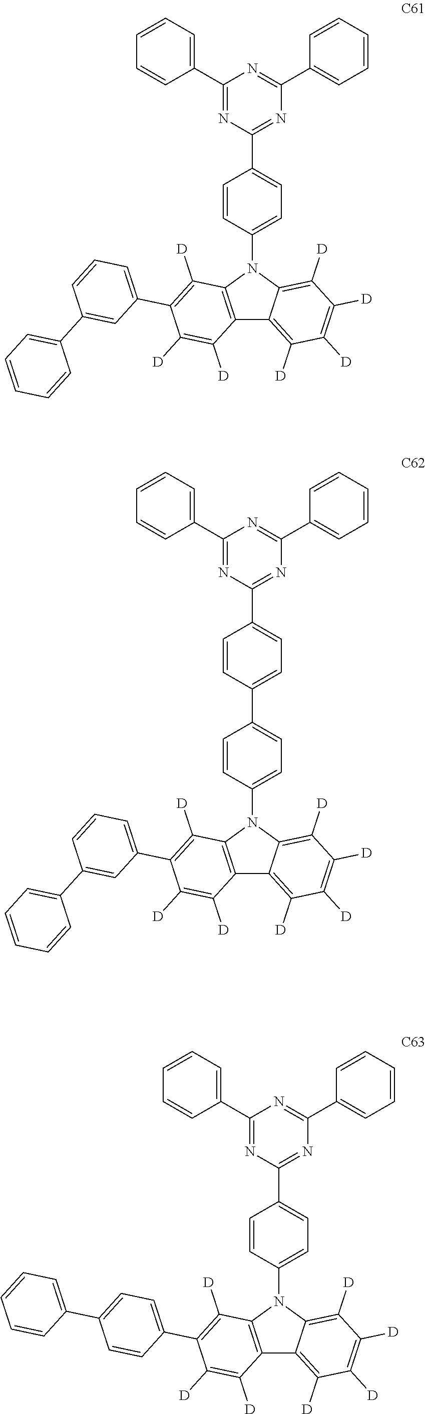 [SMILES: [2H]c1c([2H])c([2H])c2c(c1[2H])c1c([2H])c([2H])c(-c3cccc(-c4ccccc4)c3)c([2H])c1n2-c1ccc(-c2nc(-c3ccccc3)nc(-c3ccccc3)n2)cc1 ; [2H]c1c([2H])c([2H])c2c(c1[2H])c1c([2H])c([2H])c(-c3cccc(-c4ccccc4)c3)c([2H])c1n2-c1ccc(-c2ccc(-c3nc(-c4ccccc4)nc(-c4ccccc4)n3)cc2)cc1 ; [2H]c1c([2H])c([2H])c2c(c1[2H])c1c([2H])c([2H])c(-c3ccc(-c4ccccc4)cc3)c([2H])c1n2-c1ccc(-c2nc(-c3ccccc3)nc(-c3ccccc3)n2)cc1]

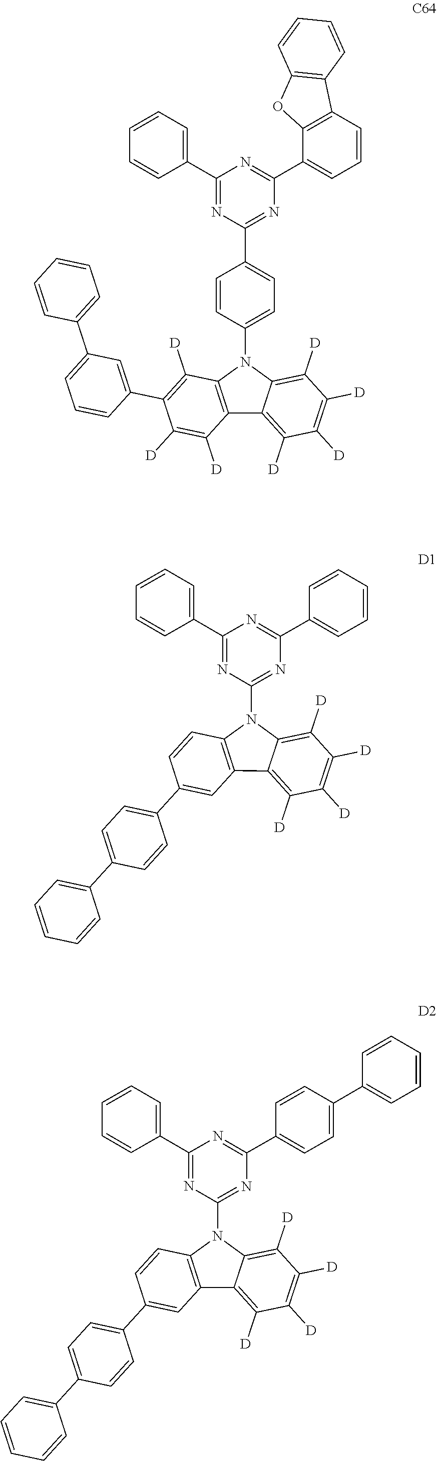 [SMILES: [2H]c1c([2H])c([2H])c2c(c1[2H])c1c([2H])c([2H])c(-c3cccc(-c4ccccc4)c3)c([2H])c1n2-c1ccc(-c2nc(-c3ccccc3)nc(-c3cccc4c3oc3ccccc34)n2)cc1 ; [2H]c1c([2H])c([2H])c2c(c1[2H])c1cc(-c3ccc(-c4ccccc4)cc3)ccc1n2-c1nc(-c2ccccc2)nc(-c2ccccc2)n1 ; [2H]c1c([2H])c([2H])c2c(c1[2H])c1cc(-c3ccc(-c4ccccc4)cc3)ccc1n2-c1nc(-c2ccccc2)nc(-c2ccc(-c3ccccc3)cc2)n1]

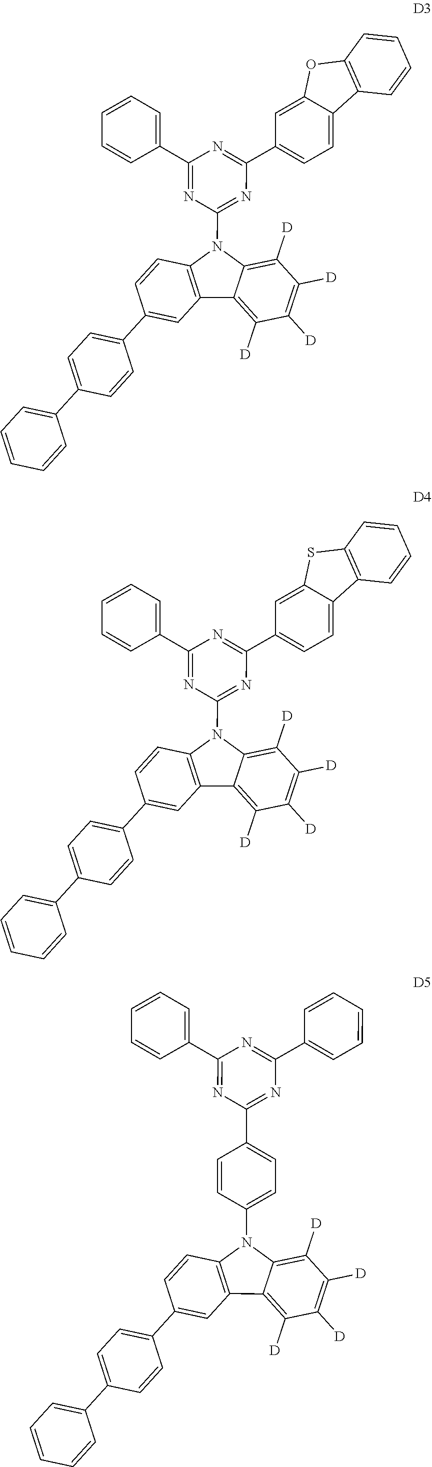 [SMILES: [2H]c1c([2H])c([2H])c2c(c1[2H])c1cc(-c3ccc(-c4ccccc4)cc3)ccc1n2-c1nc(-c2ccccc2)nc(-c2ccc3c(c2)oc2ccccc23)n1 ; [2H]c1c([2H])c([2H])c2c(c1[2H])c1cc(-c3ccc(-c4ccccc4)cc3)ccc1n2-c1nc(-c2ccccc2)nc(-c2ccc3c(c2)sc2ccccc23)n1 ; [2H]c1c([2H])c([2H])c2c(c1[2H])c1cc(-c3ccc(-c4ccccc4)cc3)ccc1n2-c1ccc(-c2nc(-c3ccccc3)nc(-c3ccccc3)n2)cc1]

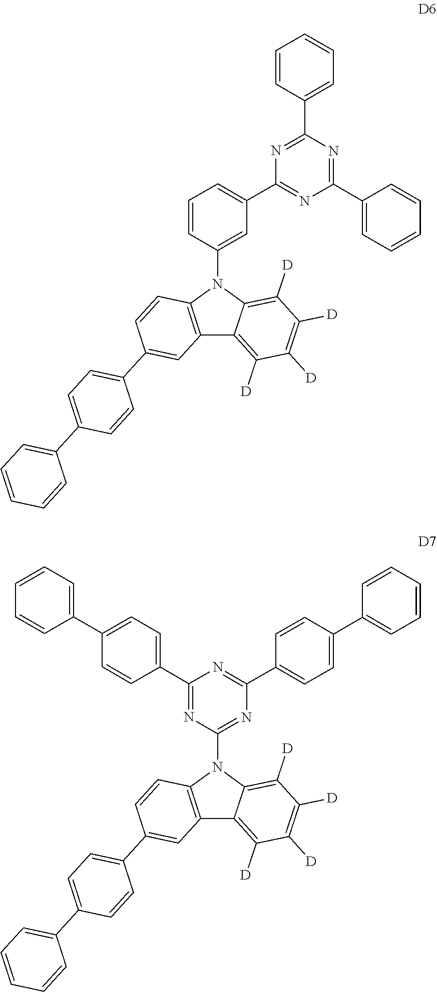 [SMILES: [2H]c1c([2H])c([2H])c2c(c1[2H])c1cc(-c3ccc(-c4ccccc4)cc3)ccc1n2-c1cccc(-c2nc(-c3ccccc3)nc(-c3ccccc3)n2)c1 ; [2H]c1c([2H])c([2H])c2c(c1[2H])c1cc(-c3ccc(-c4ccccc4)cc3)ccc1n2-c1nc(-c2ccc(-c3ccccc3)cc2)nc(-c2ccc(-c3ccccc3)cc2)n1]

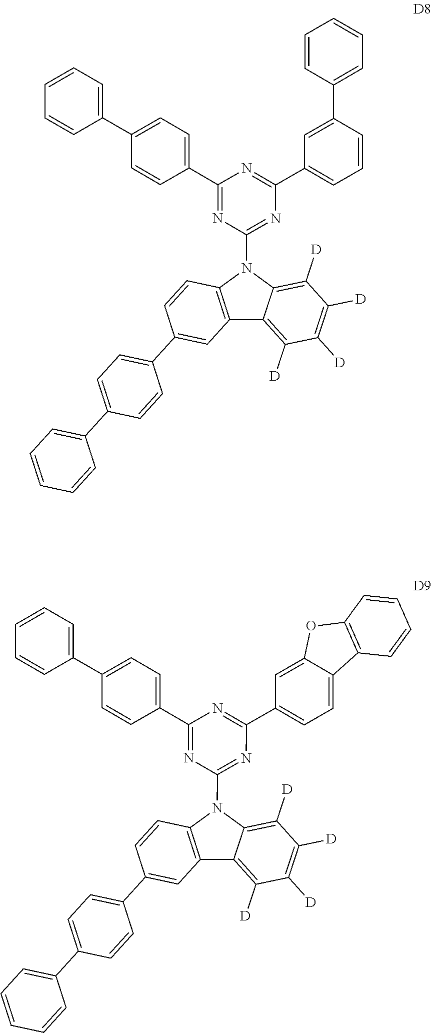 [SMILES: [2H]c1c([2H])c([2H])c2c(c1[2H])c1cc(-c3ccc(-c4ccccc4)cc3)ccc1n2-c1nc(-c2ccc(-c3ccccc3)cc2)nc(-c2cccc(-c3ccccc3)c2)n1 ; [2H]c1c([2H])c([2H])c2c(c1[2H])c1cc(-c3ccc(-c4ccccc4)cc3)ccc1n2-c1nc(-c2ccc(-c3ccccc3)cc2)nc(-c2ccc3c(c2)oc2ccccc23)n1]

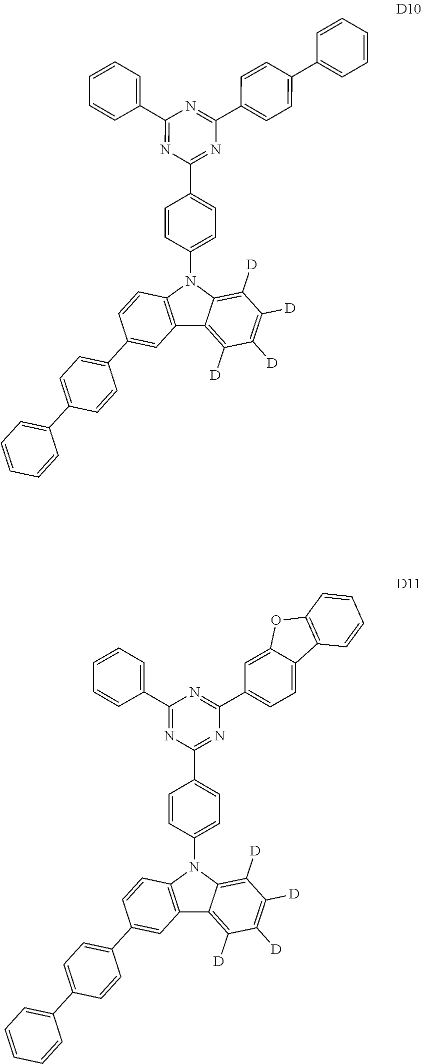 [SMILES: [2H]c1c([2H])c([2H])c2c(c1[2H])c1cc(-c3ccc(-c4ccccc4)cc3)ccc1n2-c1ccc(-c2nc(-c3ccccc3)nc(-c3ccc(-c4ccccc4)cc3)n2)cc1 ; [2H]c1c([2H])c([2H])c2c(c1[2H])c1cc(-c3ccc(-c4ccccc4)cc3)ccc1n2-c1ccc(-c2nc(-c3ccccc3)nc(-c3ccc4c(c3)oc3ccccc34)n2)cc1]

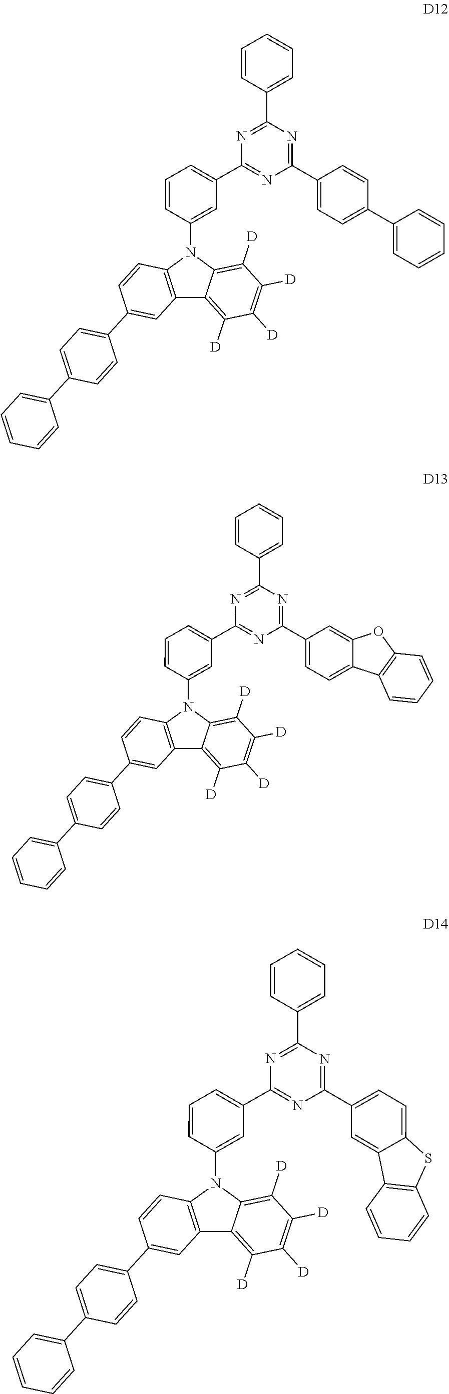 [SMILES: [2H]c1c([2H])c([2H])c2c(c1[2H])c1cc(-c3ccc(-c4ccccc4)cc3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3ccc(-c4ccccc4)cc3)ccc1n2-c1cccc(-c2nc(-c3ccccc3)nc(-c3ccc4c(c3)oc3ccccc34)n2)c1 ; [2H]c1c([2H])c([2H])c2c(c1[2H])c1cc(-c3ccc(-c4ccccc4)cc3)ccc1n2-c1cccc(-c2nc(-c3ccccc3)nc(-c3ccc4sc5ccccc5c4c3)n2)c1]

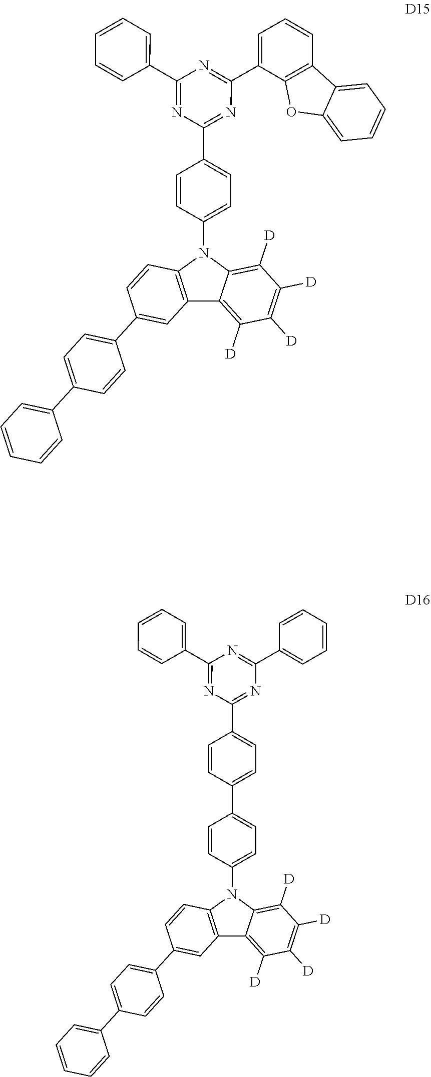 [SMILES: [2H]c1c([2H])c([2H])c2c(c1[2H])c1cc(-c3ccc(-c4ccccc4)cc3)ccc1n2-c1ccc(-c2nc(-c3ccccc3)nc(-c3cccc4c3oc3ccccc34)n2)cc1 ; [2H]c1c([2H])c([2H])c2c(c1[2H])c1cc(-c3ccc(-c4ccccc4)cc3)ccc1n2-c1ccc(-c2ccc(-c3nc(-c4ccccc4)nc(-c4ccccc4)n3)cc2)cc1]

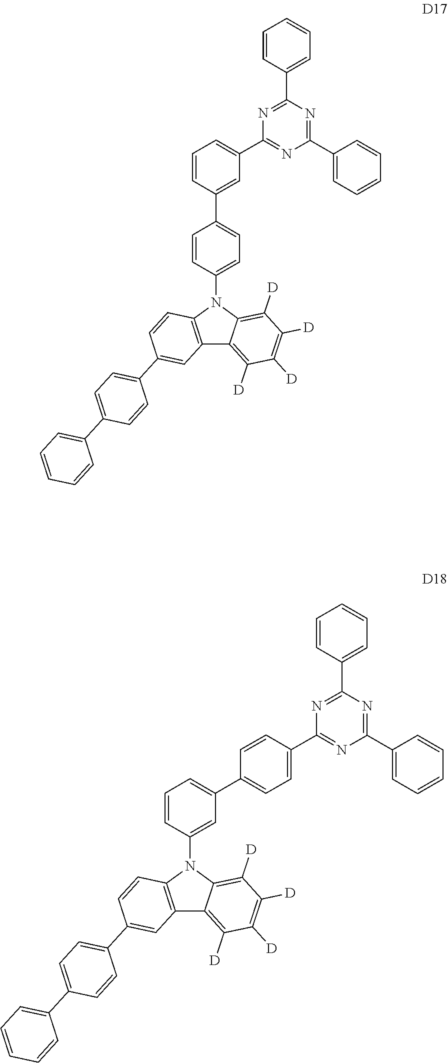 [SMILES: [2H]c1c([2H])c([2H])c2c(c1[2H])c1cc(-c3ccc(-c4ccccc4)cc3)ccc1n2-c1ccc(-c2cccc(-c3nc(-c4ccccc4)nc(-c4ccccc4)n3)c2)cc1 ; [2H]c1c([2H])c([2H])c2c(c1[2H])c1cc(-c3ccc(-c4ccccc4)cc3)ccc1n2-c1cccc(-c2ccc(-c3nc(-c4ccccc4)nc(-c4ccccc4)n3)cc2)c1]

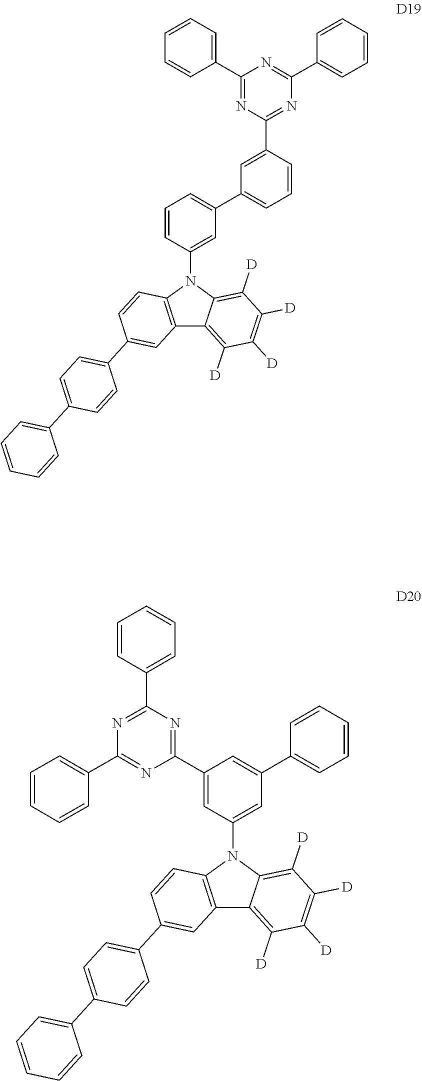 [SMILES: [2H]c1c([2H])c([2H])c2c(c1[2H])c1cc(-c3ccc(-c4ccccc4)cc3)ccc1n2-c1cccc(-c2cccc(-c3nc(-c4ccccc4)nc(-c4ccccc4)n3)c2)c1 ; [2H]c1c([2H])c([2H])c2c(c1[2H])c1cc(-c3ccc(-c4ccccc4)cc3)ccc1n2-c1cc(-c2ccccc2)cc(-c2nc(-c3ccccc3)nc(-c3ccccc3)n2)c1]

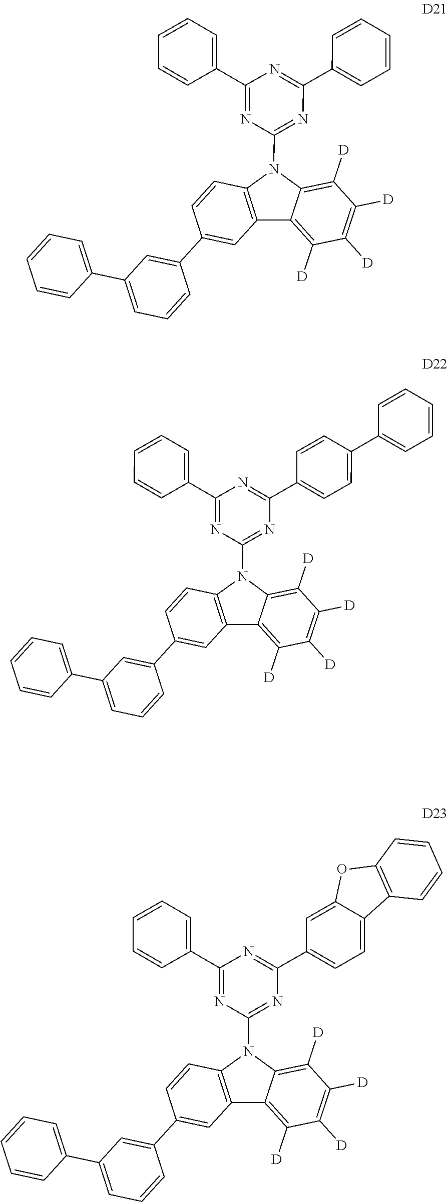 [SMILES: [2H]c1c([2H])c([2H])c2c(c1[2H])c1cc(-c3cccc(-c4ccccc4)c3)ccc1n2-c1nc(-c2ccccc2)nc(-c2ccccc2)n1 ; [2H]c1c([2H])c([2H])c2c(c1[2H])c1cc(-c3cccc(-c4ccccc4)c3)ccc1n2-c1nc(-c2ccccc2)nc(-c2ccc(-c3ccccc3)cc2)n1 ; [2H]c1c([2H])c([2H])c2c(c1[2H])c1cc(-c3cccc(-c4ccccc4)c3)ccc1n2-c1nc(-c2ccccc2)nc(-c2ccc3c(c2)oc2ccccc23)n1]

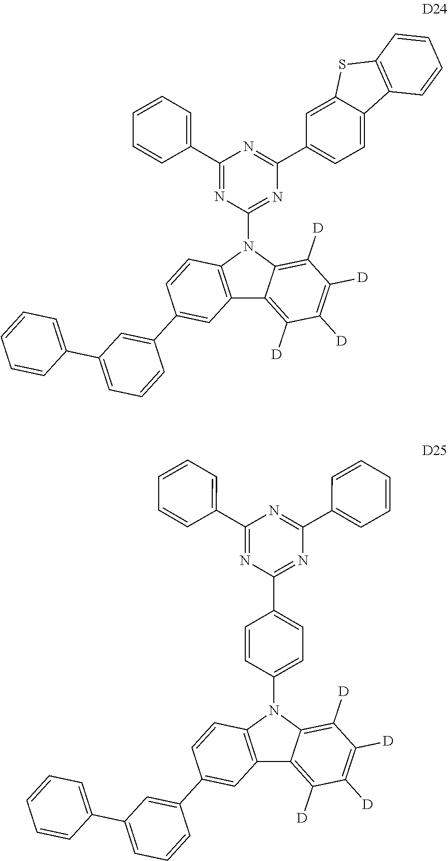 [SMILES: [2H]c1c([2H])c([2H])c2c(c1[2H])c1cc(-c3cccc(-c4ccccc4)c3)ccc1n2-c1nc(-c2ccccc2)nc(-c2ccc3c(c2)sc2ccccc23)n1 ; [2H]c1c([2H])c([2H])c2c(c1[2H])c1cc(-c3cccc(-c4ccccc4)c3)ccc1n2-c1ccc(-c2nc(-c3ccccc3)nc(-c3ccccc3)n2)cc1]

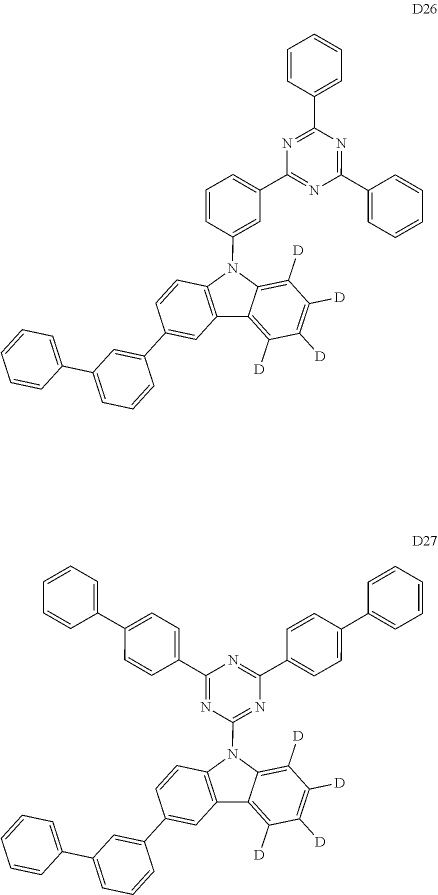 [SMILES: [2H]c1c([2H])c([2H])c2c(c1[2H])c1cc(-c3cccc(-c4ccccc4)c3)ccc1n2-c1cccc(-c2nc(-c3ccccc3)nc(-c3ccccc3)n2)c1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccc(-c3ccccc3)cc2)n1]

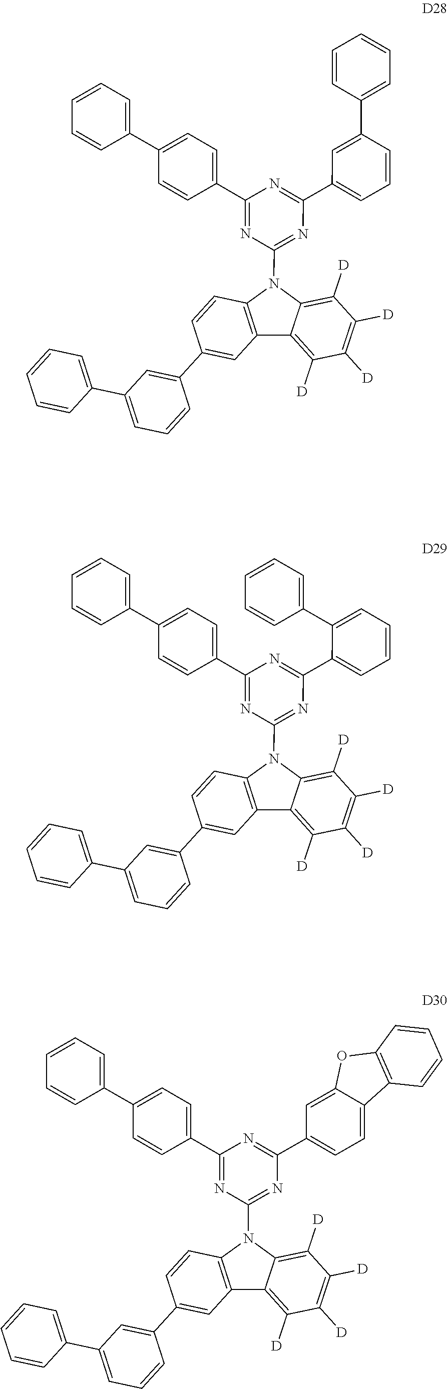 [SMILES: [2H]c1c([2H])c([2H])c2c(c1[2H])c1cc(-c3cccc(-c4ccccc4)c3)ccc1n2-c1nc(-c2ccc(-c3ccccc3)cc2)nc(-c2cccc(-c3ccccc3)c2)n1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccccc2-c2ccccc2)n1 ; [2H]c1c([2H])c([2H])c2c(c1[2H])c1cc(-c3cccc(-c4ccccc4)c3)ccc1n2-c1nc(-c2ccc(-c3ccccc3)cc2)nc(-c2ccc3c(c2)oc2ccccc23)n1]

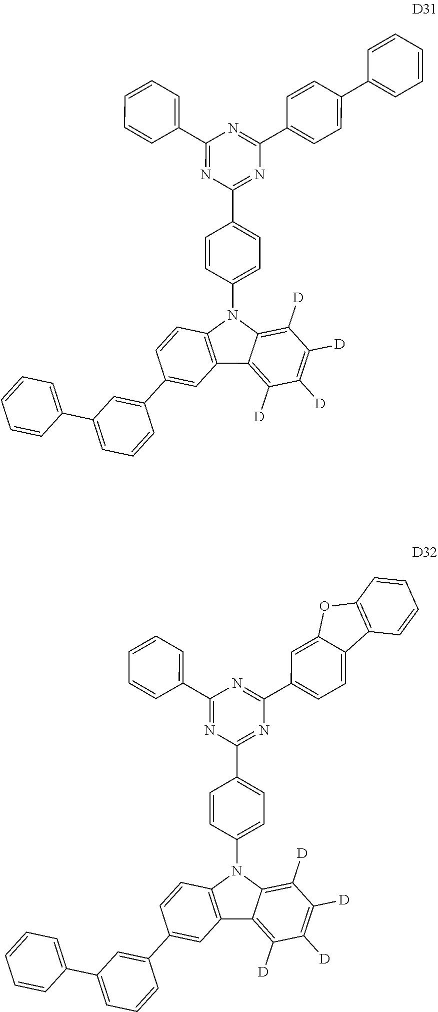 [SMILES: [2H]c1c([2H])c([2H])c2c(c1[2H])c1cc(-c3cccc(-c4ccccc4)c3)ccc1n2-c1ccc(-c2nc(-c3ccccc3)nc(-c3ccc(-c4ccccc4)cc3)n2)cc1 ; [2H]c1c([2H])c([2H])c2c(c1[2H])c1cc(-c3cccc(-c4ccccc4)c3)ccc1n2-c1ccc(-c2nc(-c3ccccc3)nc(-c3ccc4c(c3)oc3ccccc34)n2)cc1]

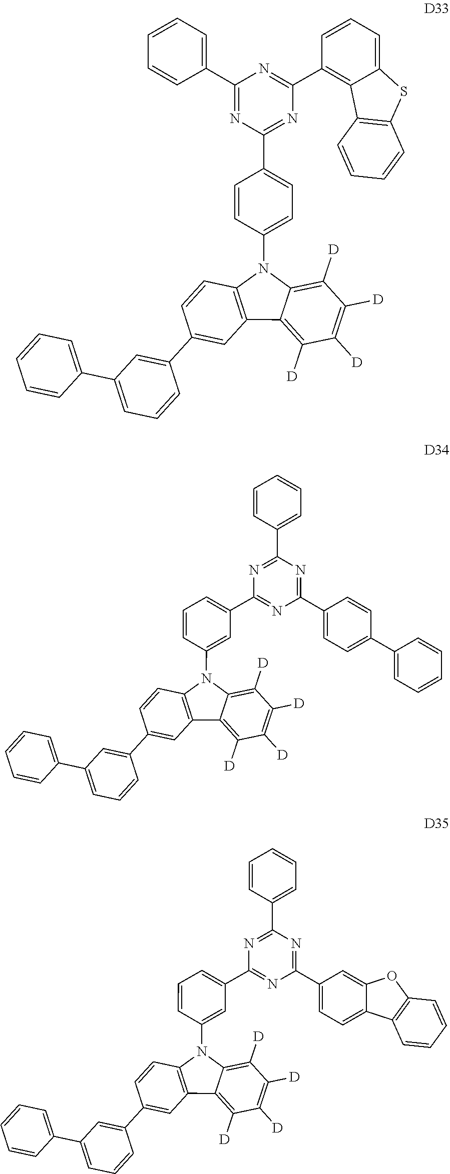 [SMILES: [2H]c1c([2H])c([2H])c2c(c1[2H])c1cc(-c3cccc(-c4ccccc4)c3)ccc1n2-c1ccc(-c2nc(-c3ccccc3)nc(-c3cccc4sc5ccccc5c34)n2)cc1 ; [2H]c1c([2H])c([2H])c2c(c1[2H])c1cc(-c3cccc(-c4ccccc4)c3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3cccc(-c4ccccc4)c3)ccc1n2-c1cccc(-c2nc(-c3ccccc3)nc(-c3ccc4c(c3)oc3ccccc34)n2)c1]

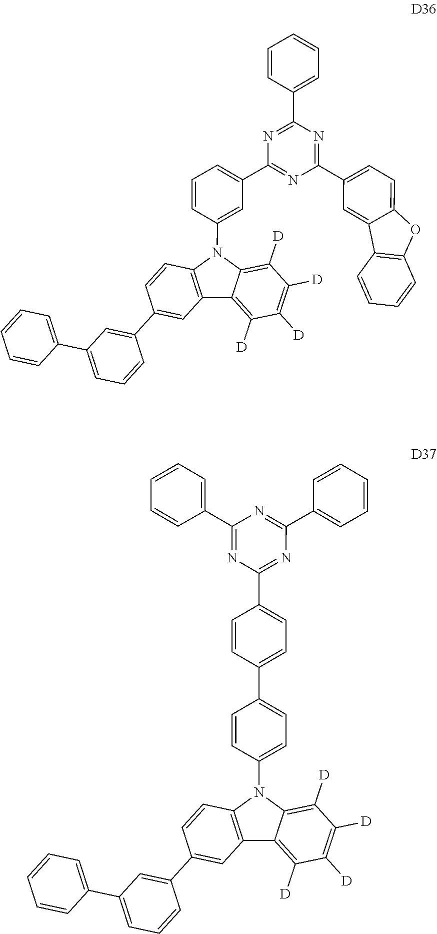 [SMILES: [2H]c1c([2H])c([2H])c2c(c1[2H])c1cc(-c3cccc(-c4ccccc4)c3)ccc1n2-c1cccc(-c2nc(-c3ccccc3)nc(-c3ccc4oc5ccccc5c4c3)n2)c1 ; [2H]c1c([2H])c([2H])c2c(c1[2H])c1cc(-c3cccc(-c4ccccc4)c3)ccc1n2-c1ccc(-c2ccc(-c3nc(-c4ccccc4)nc(-c4ccccc4)n3)cc2)cc1]

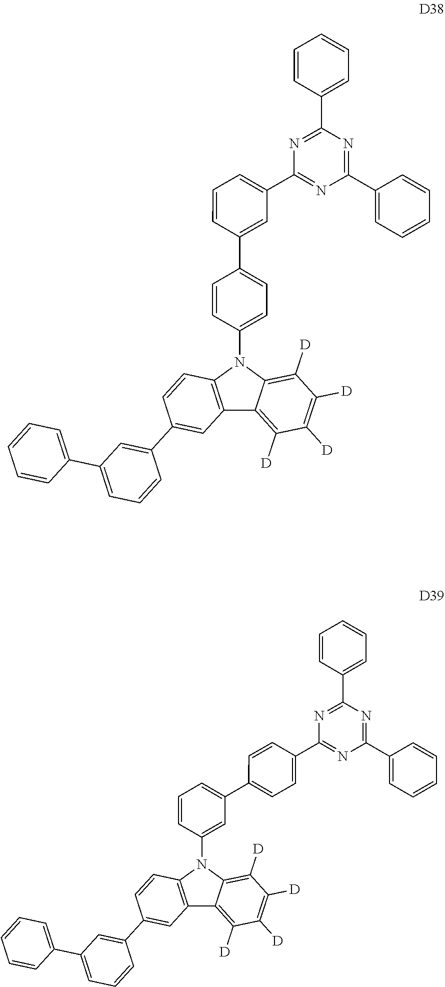 [SMILES: [2H]c1c([2H])c([2H])c2c(c1[2H])c1cc(-c3cccc(-c4ccccc4)c3)ccc1n2-c1ccc(-c2cccc(-c3nc(-c4ccccc4)nc(-c4ccccc4)n3)c2)cc1 ; [2H]c1c([2H])c([2H])c2c(c1[2H])c1cc(-c3cccc(-c4ccccc4)c3)ccc1n2-c1cccc(-c2ccc(-c3nc(-c4ccccc4)nc(-c4ccccc4)n3)cc2)c1]

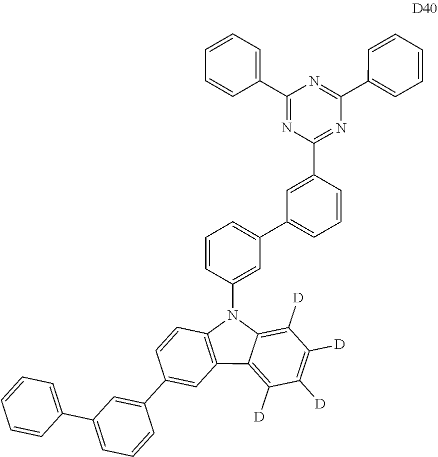 [SMILES: [2H]c1c([2H])c([2H])c2c(c1[2H])c1cc(-c3cccc(-c4ccccc4)c3)ccc1n2-c1cccc(-c2cccc(-c3nc(-c4ccccc4)nc(-c4ccccc4)n3)c2)c1]

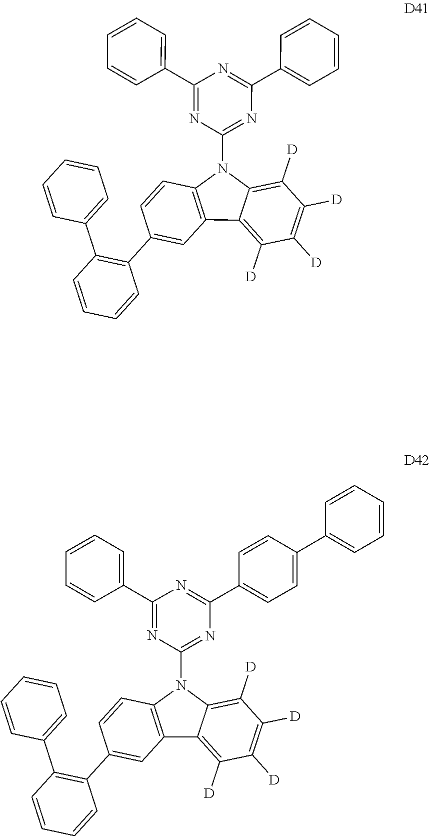 [SMILES: [2H]c1c([2H])c([2H])c2c(c1[2H])c1cc(-c3ccccc3-c3ccccc3)ccc1n2-c1nc(-c2ccccc2)nc(-c2ccccc2)n1 ; [2H]c1c([2H])c([2H])c2c(c1[2H])c1cc(-c3ccccc3-c3ccccc3)ccc1n2-c1nc(-c2ccccc2)nc(-c2ccc(-c3ccccc3)cc2)n1]

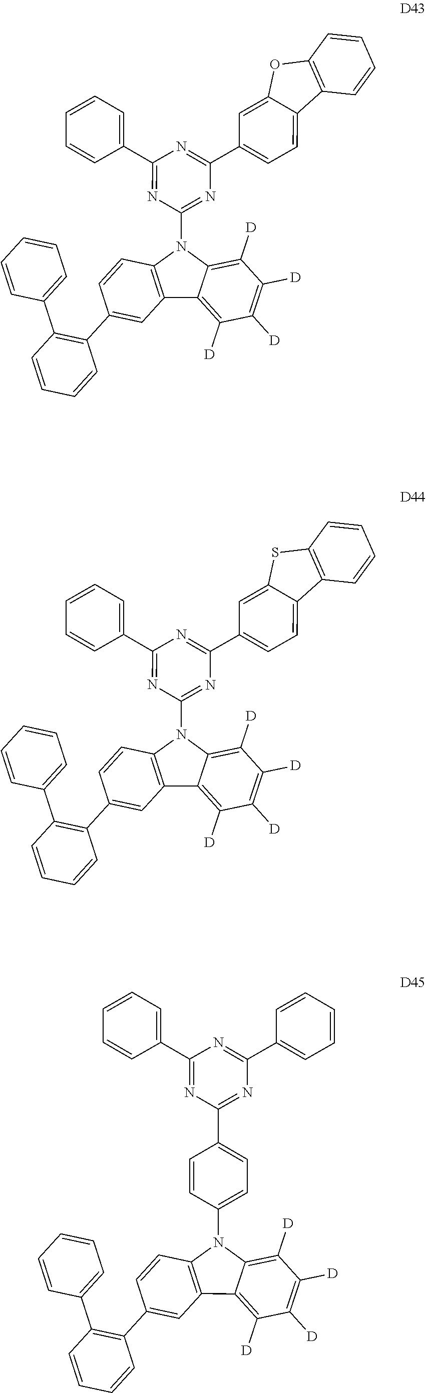 [SMILES: [2H]c1c([2H])c([2H])c2c(c1[2H])c1cc(-c3ccccc3-c3ccccc3)ccc1n2-c1nc(-c2ccccc2)nc(-c2ccc3c(c2)oc2ccccc23)n1 ; [2H]c1c([2H])c([2H])c2c(c1[2H])c1cc(-c3ccccc3-c3ccccc3)ccc1n2-c1nc(-c2ccccc2)nc(-c2ccc3c(c2)sc2ccccc23)n1 ; [2H]c1c([2H])c([2H])c2c(c1[2H])c1cc(-c3ccccc3-c3ccccc3)ccc1n2-c1ccc(-c2nc(-c3ccccc3)nc(-c3ccccc3)n2)cc1]

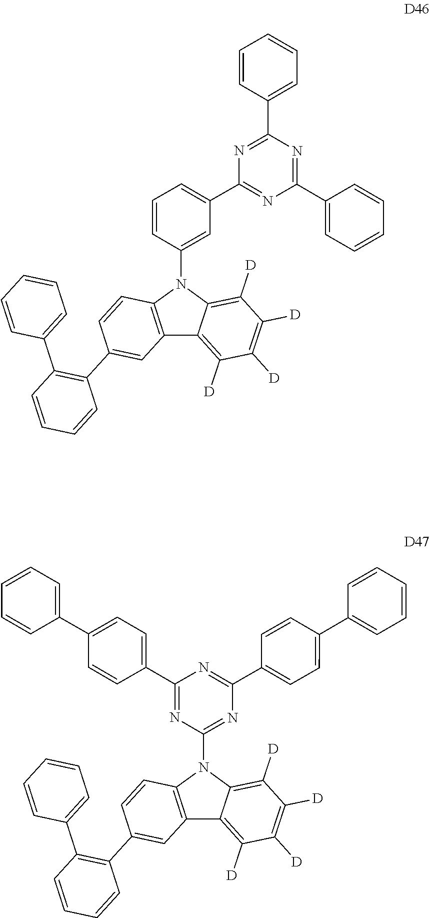 [SMILES: [2H]c1c([2H])c([2H])c2c(c1[2H])c1cc(-c3ccccc3-c3ccccc3)ccc1n2-c1cccc(-c2nc(-c3ccccc3)nc(-c3ccccc3)n2)c1 ; [2H]c1c([2H])c([2H])c2c(c1[2H])c1cc(-c3ccccc3-c3ccccc3)ccc1n2-c1nc(-c2ccc(-c3ccccc3)cc2)nc(-c2ccc(-c3ccccc3)cc2)n1]

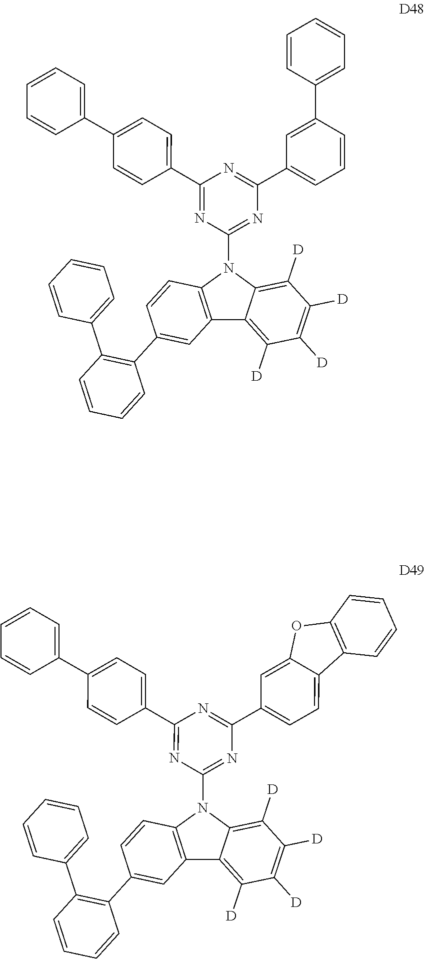 [SMILES: [2H]c1c([2H])c([2H])c2c(c1[2H])c1cc(-c3ccccc3-c3ccccc3)ccc1n2-c1nc(-c2ccc(-c3ccccc3)cc2)nc(-c2cccc(-c3ccccc3)c2)n1 ; [2H]c1c([2H])c([2H])c2c(c1[2H])c1cc(-c3ccccc3-c3ccccc3)ccc1n2-c1nc(-c2ccc(-c3ccccc3)cc2)nc(-c2ccc3c(c2)oc2ccccc23)n1]

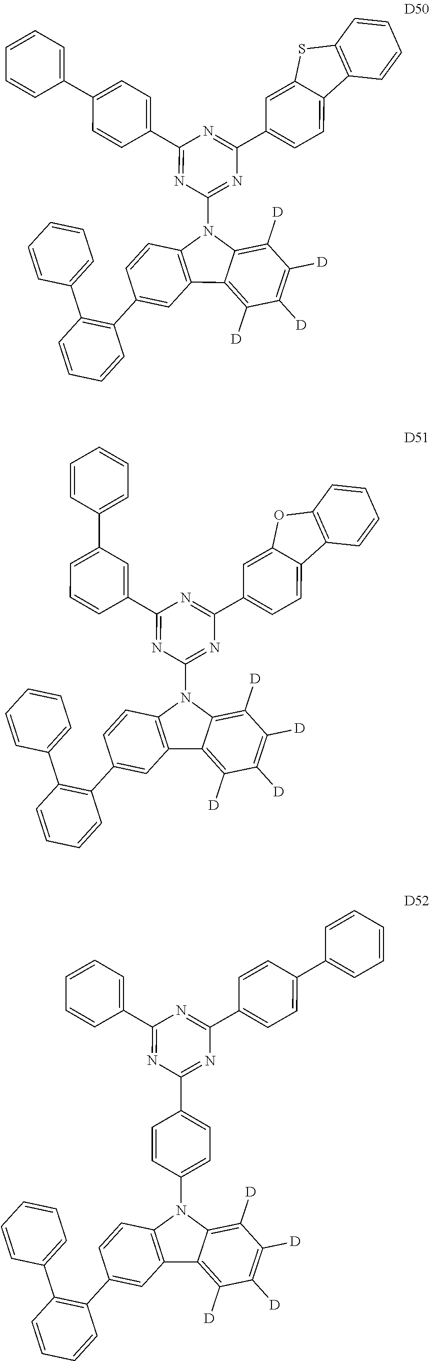 [SMILES: [2H]c1c([2H])c([2H])c2c(c1[2H])c1cc(-c3ccccc3-c3ccccc3)ccc1n2-c1nc(-c2ccc(-c3ccccc3)cc2)nc(-c2ccc3c(c2)sc2ccccc23)n1 ; [2H]c1c([2H])c([2H])c2c(c1[2H])c1cc(-c3ccccc3-c3ccccc3)ccc1n2-c1nc(-c2cccc(-c3ccccc3)c2)nc(-c2ccc3c(c2)oc2ccccc23)n1 ; [2H]c1c([2H])c([2H])c2c(c1[2H])c1cc(-c3ccccc3-c3ccccc3)ccc1n2-c1ccc(-c2nc(-c3ccccc3)nc(-c3ccc(-c4ccccc4)cc3)n2)cc1]

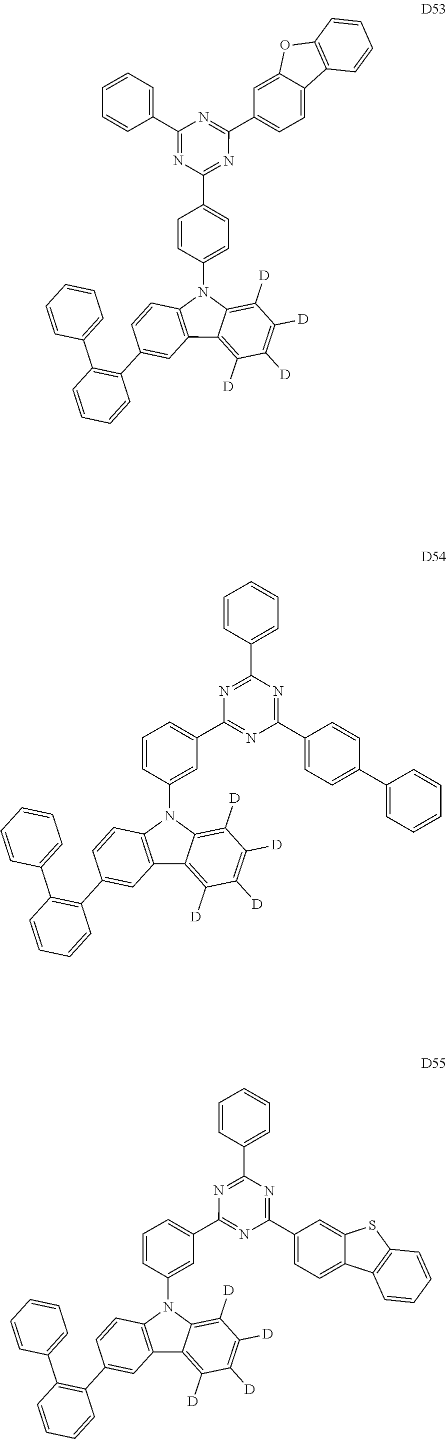 [SMILES: [2H]c1c([2H])c([2H])c2c(c1[2H])c1cc(-c3ccccc3-c3ccccc3)ccc1n2-c1ccc(-c2nc(-c3ccccc3)nc(-c3ccc4c(c3)oc3ccccc34)n2)cc1 ; [2H]c1c([2H])c([2H])c2c(c1[2H])c1cc(-c3ccccc3-c3ccccc3)ccc1n2-c1cccc(-c2nc(-c3ccccc3)nc(-c3ccc(-c4ccccc4)cc3)n2)c1 ; [2H]c1c([2H])c([2H])c2c(c1[2H])c1cc(-c3ccccc3-c3ccccc3)ccc1n2-c1cccc(-c2nc(-c3ccccc3)nc(-c3ccc4c(c3)sc3ccccc34)n2)c1]

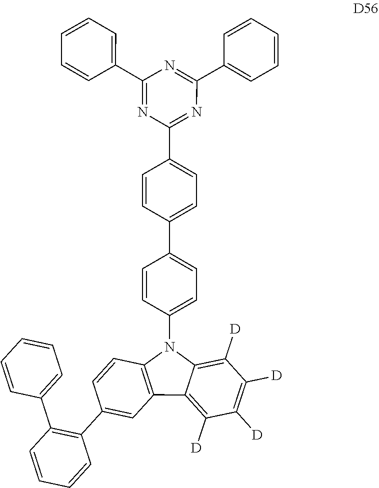 [SMILES: [2H]c1c([2H])c([2H])c2c(c1[2H])c1cc(-c3ccccc3-c3ccccc3)ccc1n2-c1ccc(-c2ccc(-c3nc(-c4ccccc4)nc(-c4ccccc4)n3)cc2)cc1]

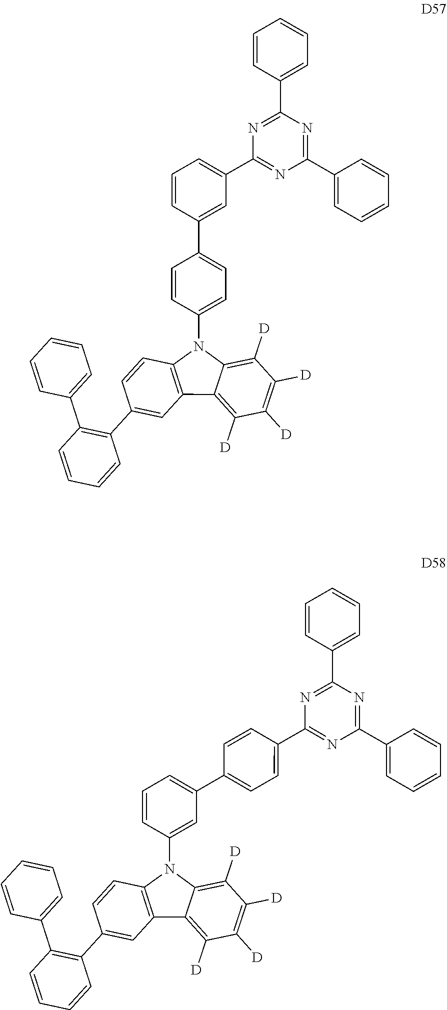 [SMILES: [2H]c1c([2H])c([2H])c2c(c1[2H])c1cc(-c3ccccc3-c3ccccc3)ccc1n2-c1ccc(-c2cccc(-c3nc(-c4ccccc4)nc(-c4ccccc4)n3)c2)cc1 ; [2H]c1c([2H])c([2H])c2c(c1[2H])c1cc(-c3ccccc3-c3ccccc3)ccc1n2-c1cccc(-c2ccc(-c3nc(-c4ccccc4)nc(-c4ccccc4)n3)cc2)c1]

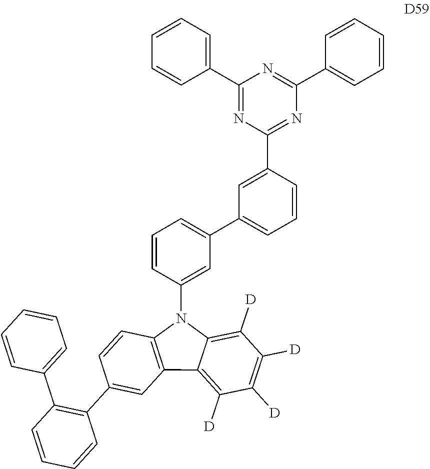 [SMILES: [2H]c1c([2H])c([2H])c2c(c1[2H])c1cc(-c3ccccc3-c3ccccc3)ccc1n2-c1cccc(-c2cccc(-c3nc(-c4ccccc4)nc(-c4ccccc4)n3)c2)c1]

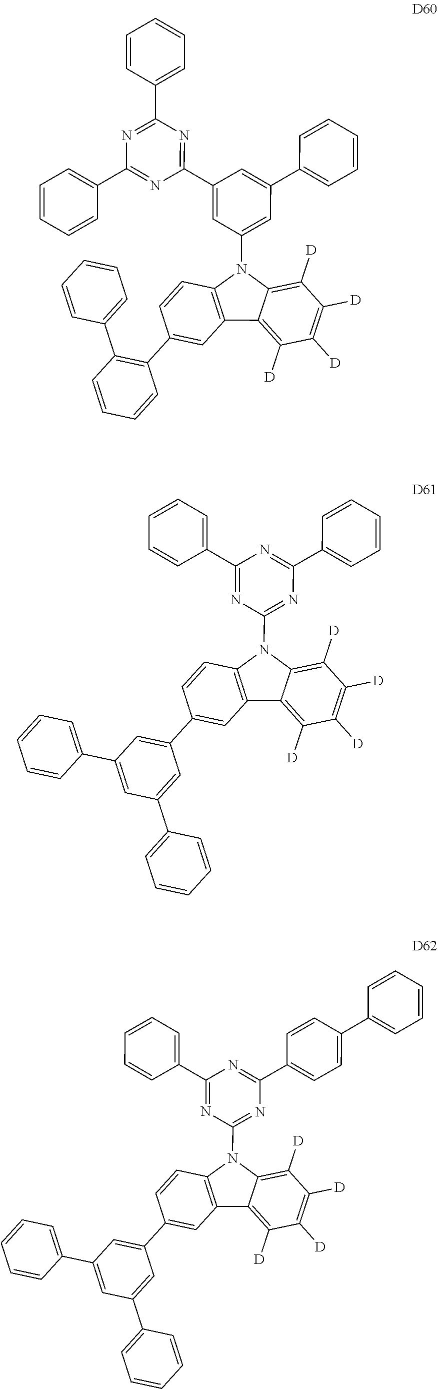 [SMILES: [2H]c1c([2H])c([2H])c2c(c1[2H])c1cc(-c3ccccc3-c3ccccc3)ccc1n2-c1cc(-c2ccccc2)cc(-c2nc(-c3ccccc3)nc(-c3ccccc3)n2)c1 ; [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccccc2)n1 ; [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccc(-c3ccccc3)cc2)n1]

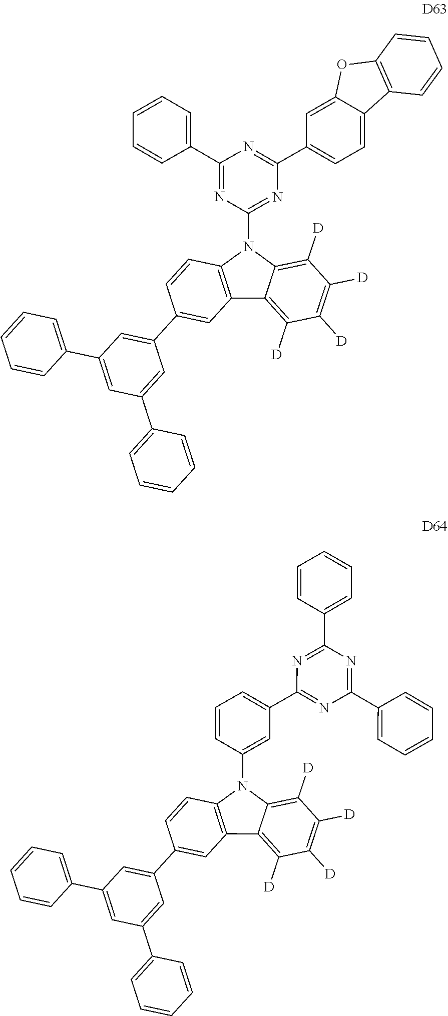 [SMILES: [2H]c1c([2H])c([2H])c2c(c1[2H])c1cc(-c3cc(-c4ccccc4)cc(-c4ccccc4)c3)ccc1n2-c1nc(-c2ccccc2)nc(-c2ccc3c(c2)oc2ccccc23)n1 ; [2H]c1c([2H])c([2H])c2c(c1[2H])c1cc(-c3cc(-c4ccccc4)cc(-c4ccccc4)c3)ccc1n2-c1cccc(-c2nc(-c3ccccc3)nc(-c3ccccc3)n2)c1]

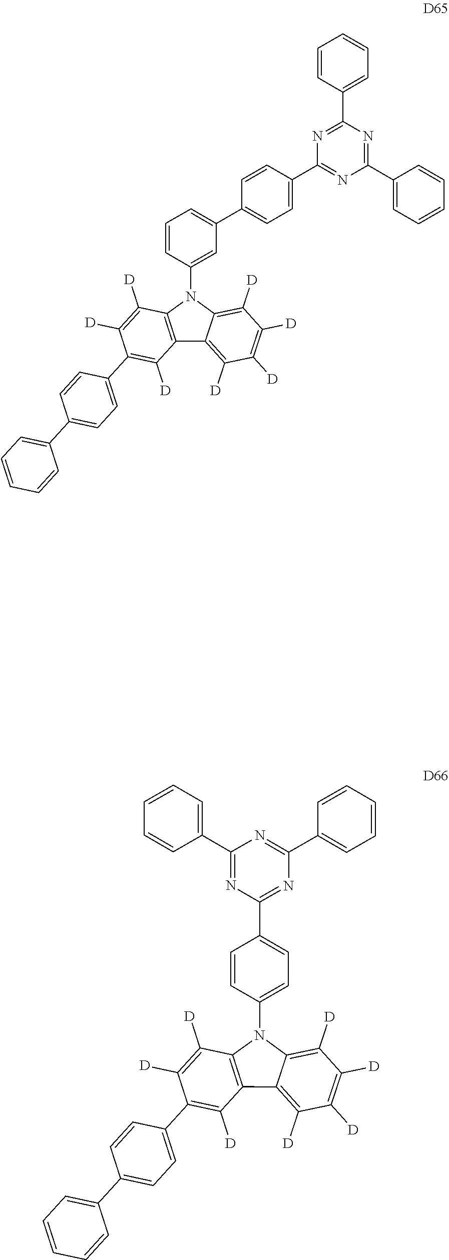 [SMILES: [2H]c1c([2H])c([2H])c2c(c1[2H])c1c([2H])c(-c3ccc(-c4ccccc4)cc3)c([2H])c([2H])c1n2-c1cccc(-c2ccc(-c3nc(-c4ccccc4)nc(-c4ccccc4)n3)cc2)c1 ; [2H]c1c([2H])c([2H])c2c(c1[2H])c1c([2H])c(-c3ccc(-c4ccccc4)cc3)c([2H])c([2H])c1n2-c1ccc(-c2nc(-c3ccccc3)nc(-c3ccccc3)n2)cc1]

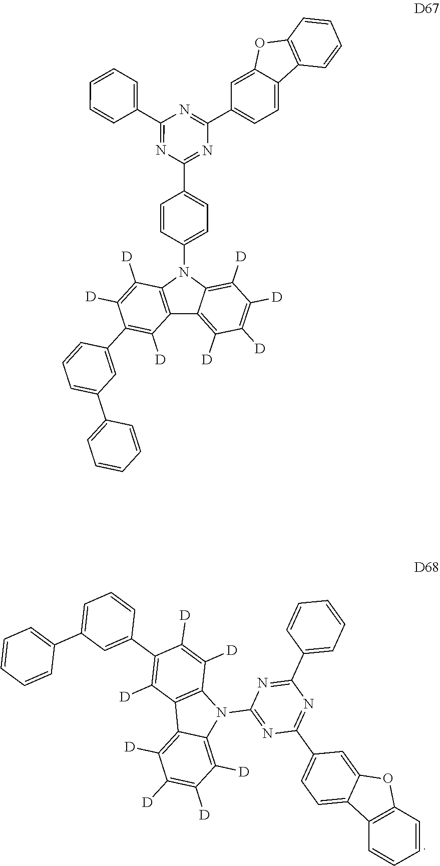 [SMILES: [2H]c1c([2H])c([2H])c2c(c1[2H])c1c([2H])c(-c3cccc(-c4ccccc4)c3)c([2H])c([2H])c1n2-c1ccc(-c2nc(-c3ccccc3)nc(-c3ccc4c(c3)oc3ccccc34)n2)cc1 ; [2H]c1c([2H])c([2H])c2c(c1[2H])c1c([2H])c(-c3cccc(-c4ccccc4)c3)c([2H])c([2H])c1n2-c1nc(-c2ccccc2)nc(-c2ccc3c(c2)oc2ccccc23)n1]

9. A composition, wherein the composition comprises a first compound and a second compound;
the first compound is selected from the organic compound according to claim 1, and the second compound is selected from a compound shown in a Formula 2: 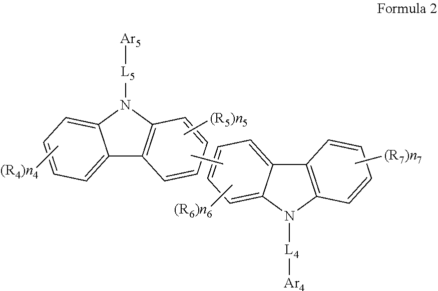
wherein each R₄, each R₅, each R₆, and each R₇ are respectively and independently selected from a hydrogen, a deuterium, a halogen group, a cyano, an aryl with 6 to 20 carbon atoms, a deuteroaryl with 6 to 20 carbon atoms, an alkyl with 1 to 10 carbon atoms, a deuteroalkyl with 1 to 10 carbon atoms, a haloalkyl with 1 to 10 carbon atoms, and a cycloalkyl with 3 to 10 carbon atoms;
n₄ represents the number of substituents R₄, n₄ is selected from 1, 2, 3 or 4, and in the case where n₄ is greater than 1, any two R₄ are the same or different;
n₅ represents the number of substituents R₅, n₅ is selected from 1, 2 or 3, and in the case where n₅ is greater than 1, any two R₅ are the same or different;
n₆ represents the number of substituents R₆, n₆ is selected from 1, 2 or 3, and in the case where n₆ is greater than 1, any two R₆ are the same or different;
n₇ represents the number of substituents R₇, n₇ is selected from 1, 2, 3 or 4, and in the case where n₇ is greater than 1, any two R₇ are the same or different;
L₄ and L₅ are the same or different, and are respectively and independently selected from a single bond, a substituted or unsubstituted arylene with 6 to 30 carbon atoms, and a substituted or unsubstituted heteroarylene with 3 to 30 carbon atoms;
Ar₄ and Ar₅ are the same or different, and are respectively and independently selected from a substituted or unsubstituted aryl with 6 to 30 carbon atoms, and a substituted or unsubstituted heteroaryl with 3 to 30 carbon atoms; and
substituent(s) in L₄, L₅, Ar₄ and Ar₅ are the same or different, and, and are respectively and independently selected from a deuterium, a halogen group, a cyano, a heteroaryl with 3 to 20 carbon atoms, an aryl with 6 to 20 carbon atoms, a deuteroaryl with 6 to 20 carbon atoms, a trialkylsilyl with 3 to 12 carbon atoms, an alkyl with 1 to 10 carbon atoms, a deuteroalkyl with 1 to 10 carbon atoms, a haloalkyl with 1 to 10 carbon atoms, a cycloalkyl with 3 to 10 carbon atoms, a heterocycloalkyl with 2 to 10 carbon atoms, and an alkoxy with 1 to 10 carbon atoms.

10. The composition according to claim 9, wherein in the second compound, each R₄, each R₅, each R₆, and each R₇ are respectively and independently selected from a hydrogen, a deuterium, a fluorine, a methyl, an ethyl, a n-propyl, an isopropyl, a tert-butyl, a phenyl, a naphthyl, a biphenyl, and a pentadeuterophenyl.

11. The composition according to claim 9, wherein in the second compound, L₄ and L₅ are respectively and independently selected from a single bond, a substituted or unsubstituted phenylene, a substituted or unsubstituted naphthylene, a substituted or unsubstituted biphenylene, a substituted or unsubstituted dibenzofurylene, a substituted or unsubstituted dibenzothienylene, and a substituted or unsubstituted carbazolylene; and
optionally, substituent(s) in L₄ and L₅ are respectively and independently selected from a deuterium, a fluorine, a cyano, a methyl, an ethyl, a n-propyl, an isopropyl, a tert-butyl, a phenyl, and a pentadeuterophenyl.

12. The composition according to claim 9, wherein in the second compound, Ar₄ and Ar₅ are respectively and independently selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted dibenzofuranyl, a substituted or unsubstituted dibenzothienyl, a substituted or unsubstituted carbazolyl and a substituted or unsubstituted triphenylene; and
optionally, substituent(s) in Ar₄ and Ar₅ are respectively and independently selected from a deuterium, a fluorine, a cyano, a halogen group, a methyl, an ethyl, a n-propyl, an isopropyl, a tert-butyl, a phenyl, and a pentadeuterophenyl.

13. The composition according to claim 9, wherein the second compound is selected from a group consisting of the following compounds:

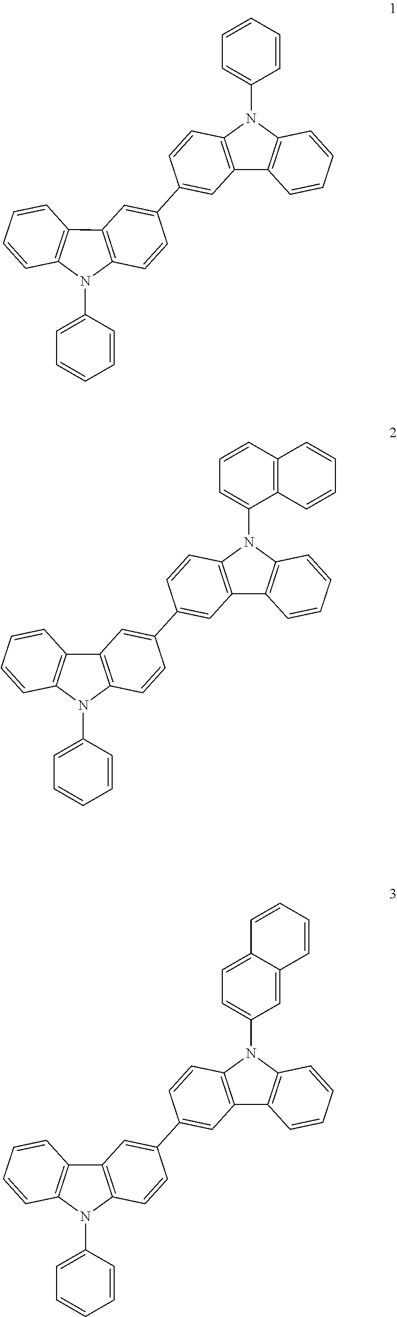 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1]

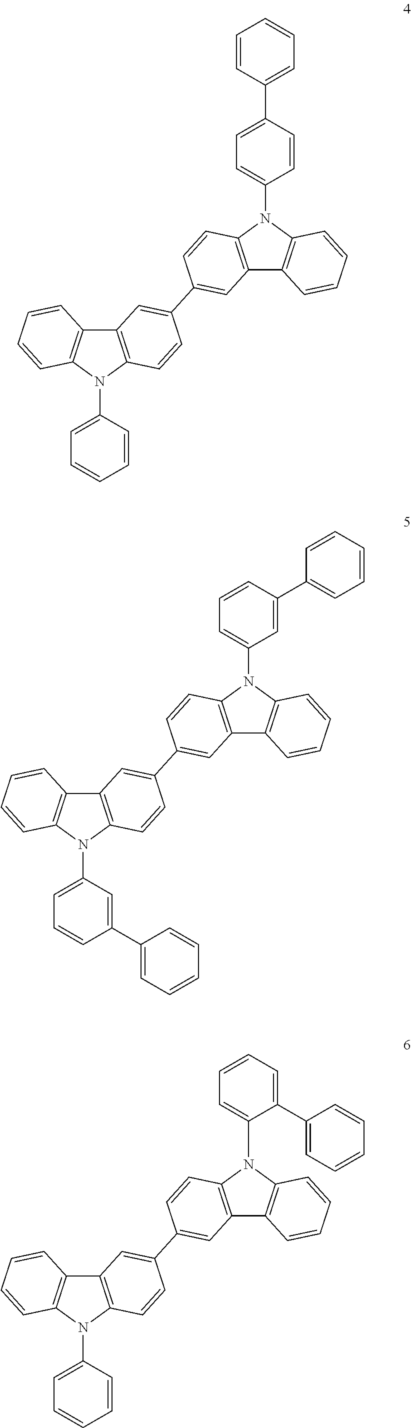 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccccc2-n2c3ccccc3c3cc(-c4ccc5c(c4)c4ccccc4n5-c4ccccc4)ccc32)cc1]

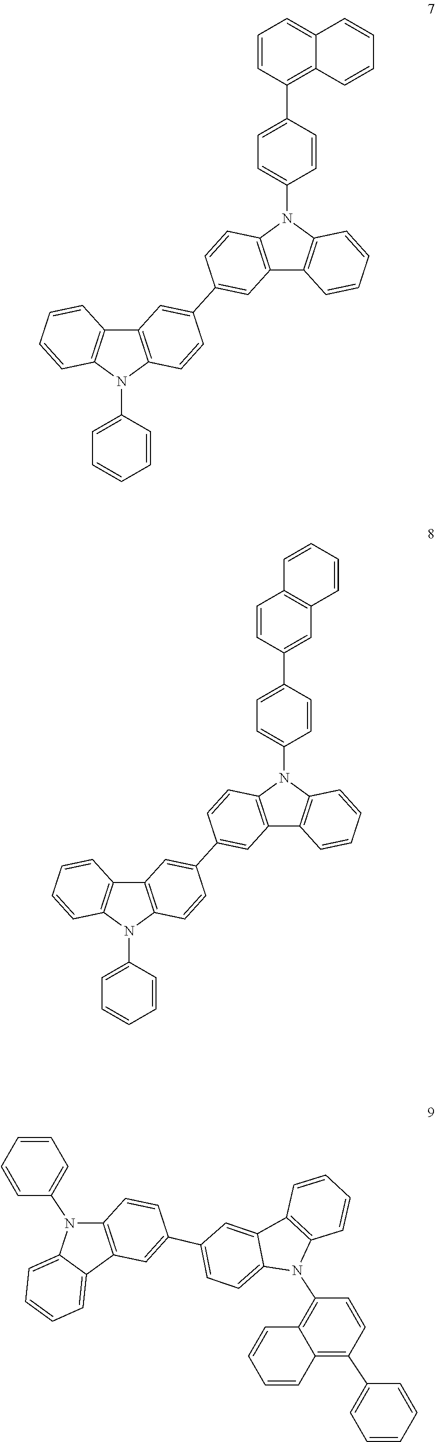 [SMILES: c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5ccccc5n6-c5ccccc5)ccc43)c3ccccc23)cc1]

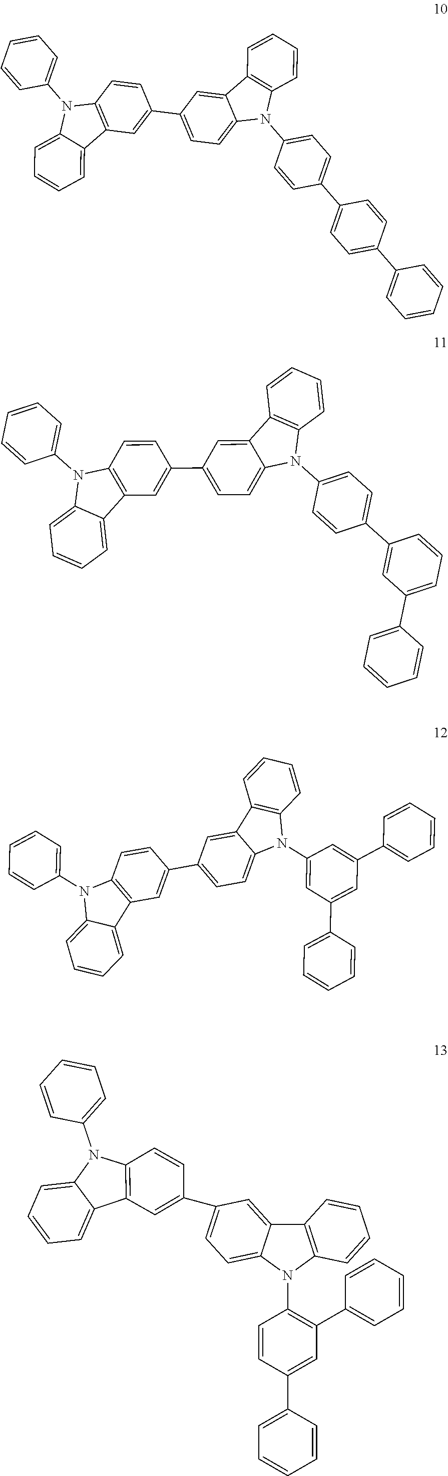 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)c(-c3ccccc3)c2)cc1]

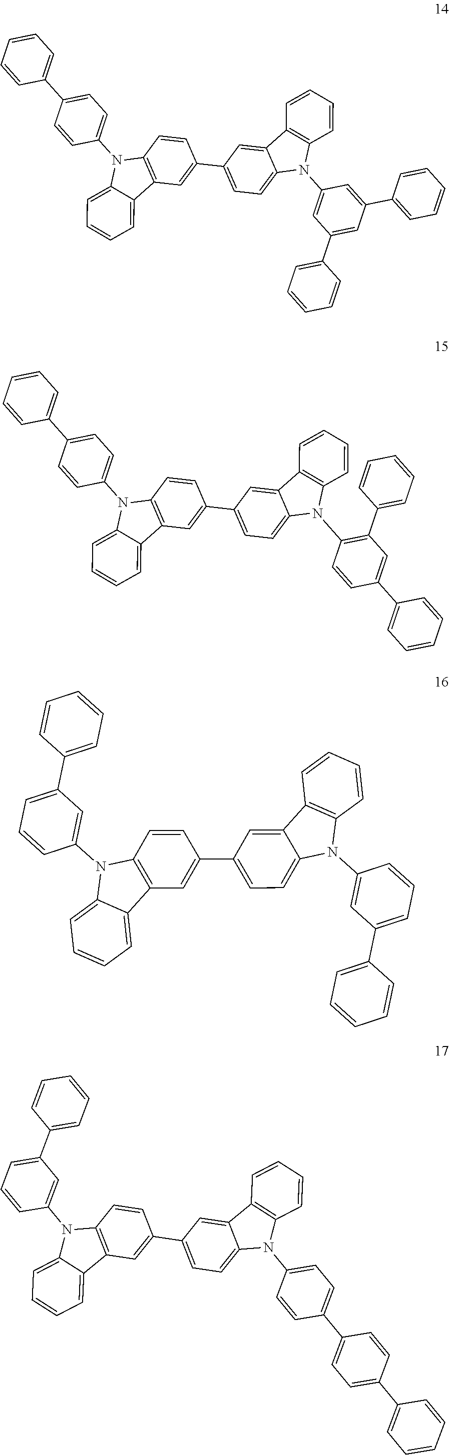 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

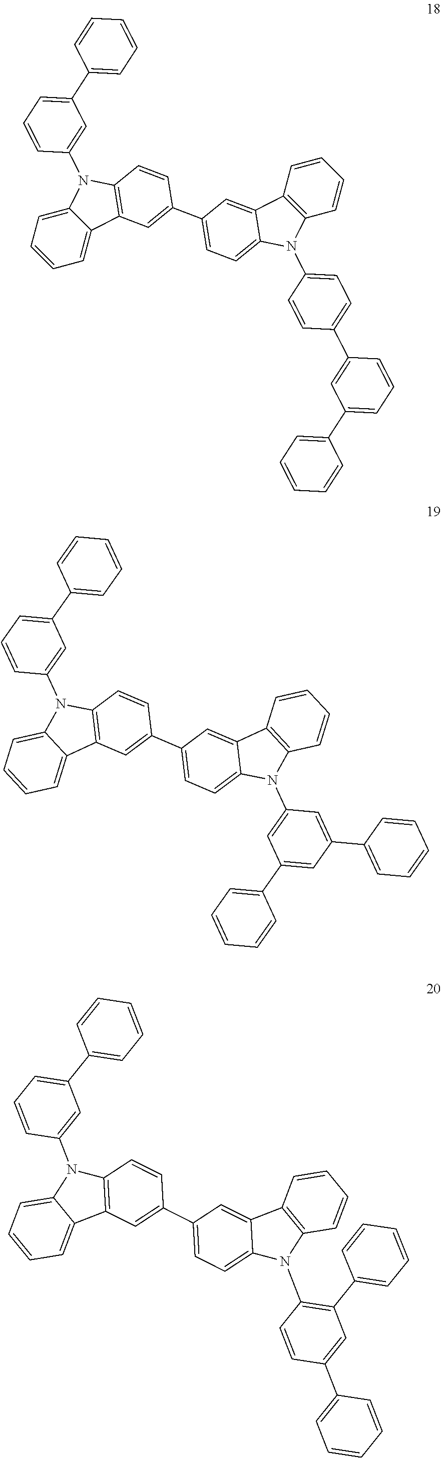 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc(-c6ccccc6)cc5-c5ccccc5)ccc43)c2)cc1]

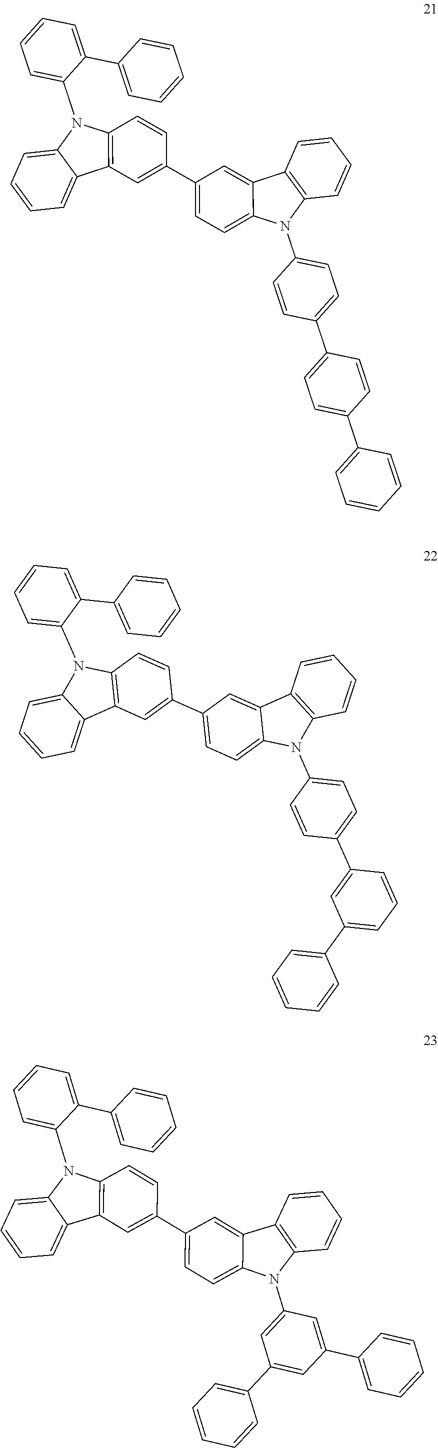 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cccc(-c3ccc(-n4c5ccccc5c5cc(-c6ccc7c(c6)c6ccccc6n7-c6ccccc6-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

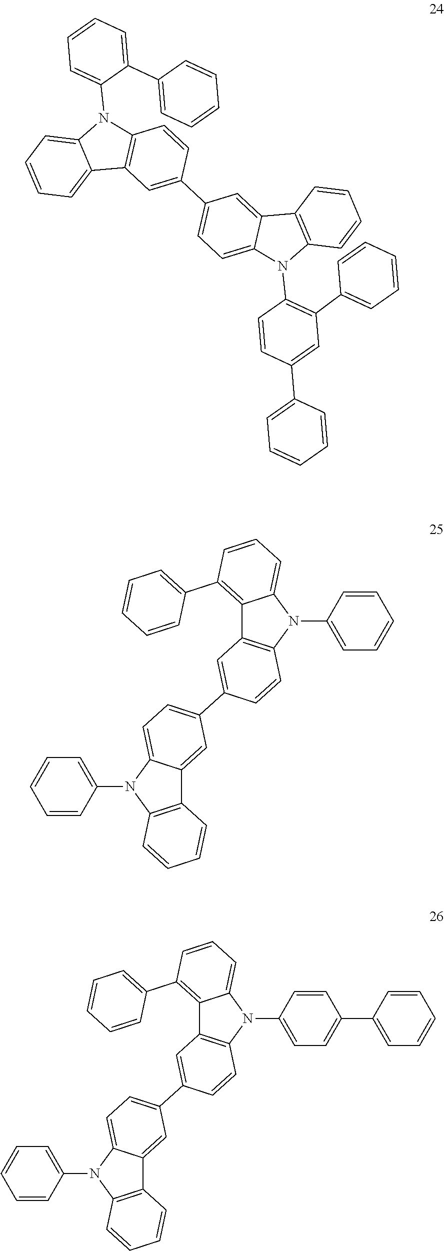 [SMILES: c1ccc(-c2ccc(-n3c4ccccc4c4cc(-c5ccc6c(c5)c5ccccc5n6-c5ccccc5-c5ccccc5)ccc43)c(-c3ccccc3)c2)cc1 ; c1ccc(-c2cccc3c2c2cc(-c4ccc5c(c4)c4ccccc4n5-c4ccccc4)ccc2n3-c2ccccc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

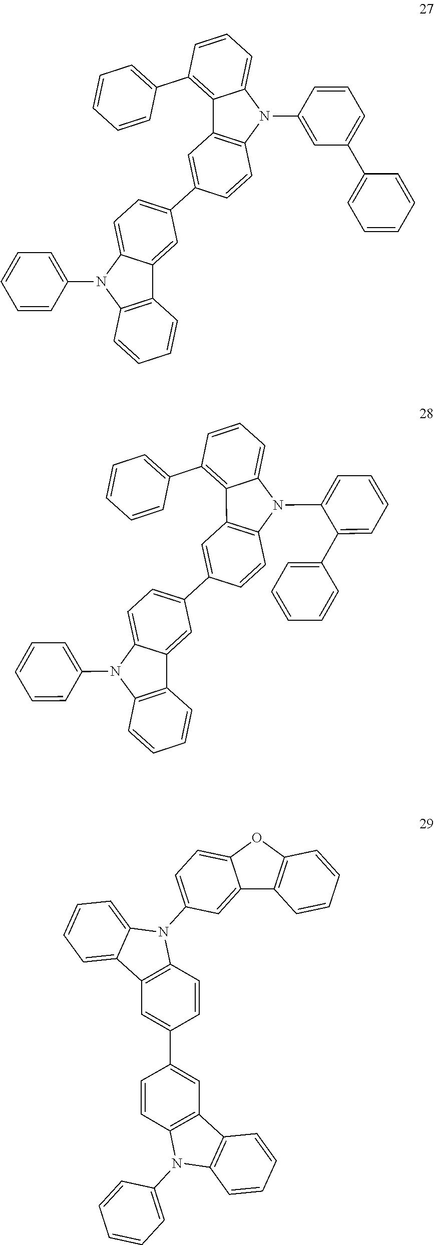 [SMILES: c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2ccccc2-n2c3ccc(-c4ccc5c(c4)c4ccccc4n5-c4ccccc4)cc3c3c(-c4ccccc4)cccc32)cc1 ; c1ccc(-n2c3ccccc3c3cc(-c4ccc5c(c4)c4ccccc4n5-c4ccc5oc6ccccc6c5c4)ccc32)cc1]

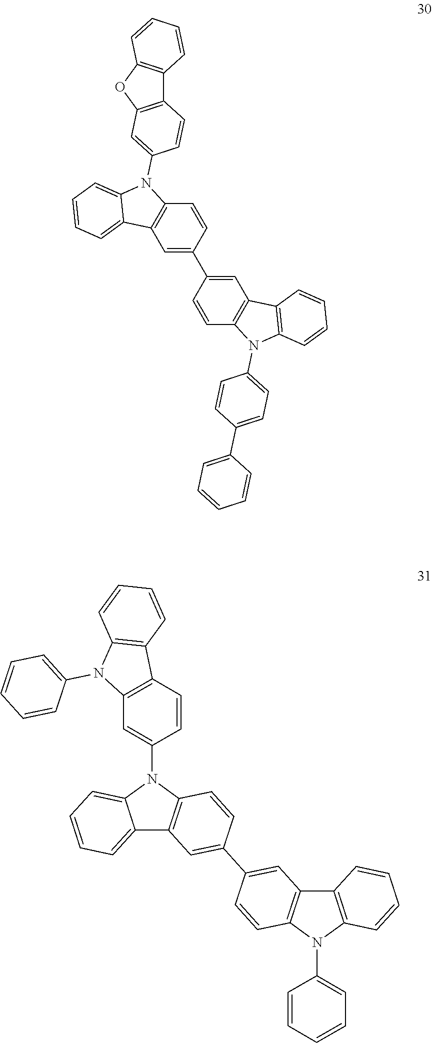

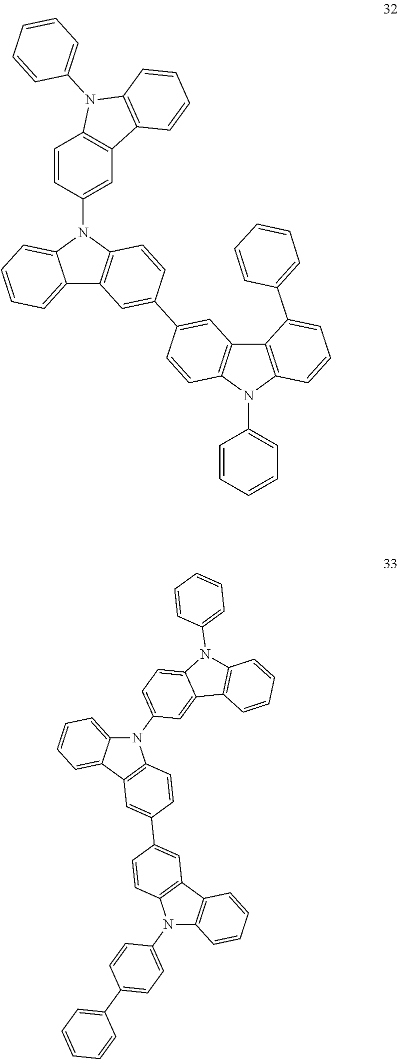 [SMILES: c1ccc(-c2cccc3c2c2cc(-c4ccc5c(c4)c4ccccc4n5-c4ccc5c(c4)c4ccccc4n5-c4ccccc4)ccc2n3-c2ccccc2)cc1 ; c1ccc(-c2ccc(-n3c4ccccc4c4cc(-c5ccc6c(c5)c5ccccc5n6-c5ccc6c(c5)c5ccccc5n6-c5ccccc5)ccc43)cc2)cc1]

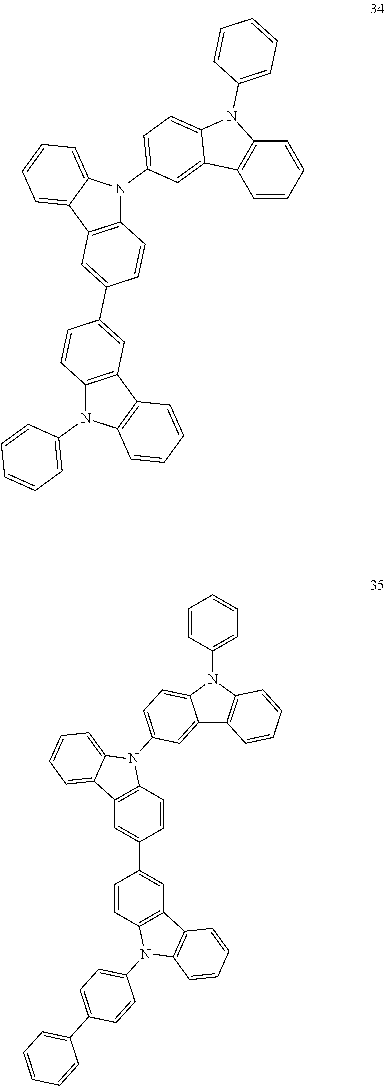 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc6c(c5)c5ccccc5n6-c5ccccc5)ccc43)cc2)cc1]

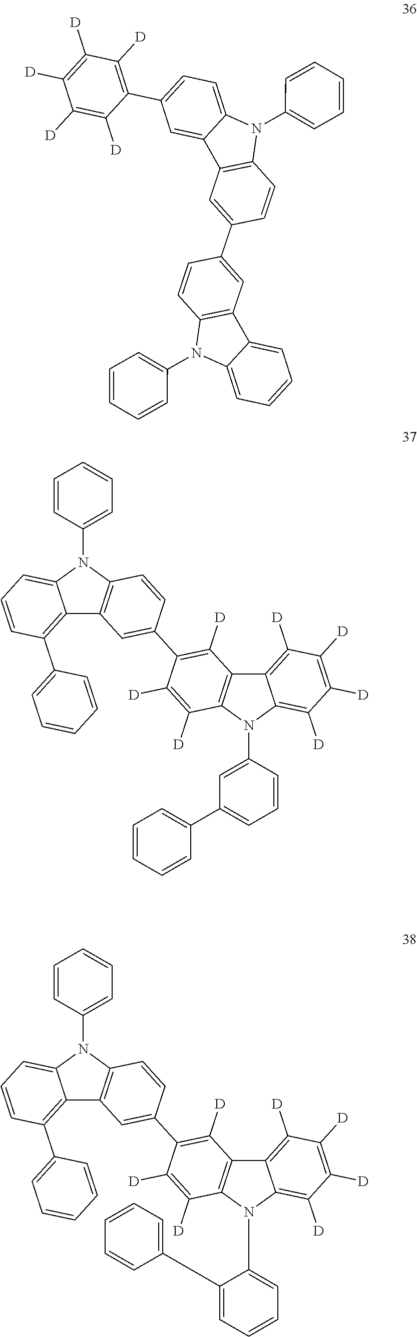 [SMILES: [2H]c1c([2H])c([2H])c(-c2ccc3c(c2)c2cc(-c4ccc5c(c4)c4ccccc4n5-c4ccccc4)ccc2n3-c2ccccc2)c([2H])c1[2H] ; [2H]c1c([2H])c([2H])c2c(c1[2H])c1c([2H])c(-c3ccc4c(c3)c3c(-c5ccccc5)cccc3n4-c3ccccc3)c([2H])c([2H])c1n2-c1cccc(-c2ccccc2)c1 ; [2H]c1c([2H])c([2H])c2c(c1[2H])c1c([2H])c(-c3ccc4c(c3)c3c(-c5ccccc5)cccc3n4-c3ccccc3)c([2H])c([2H])c1n2-c1ccccc1-c1ccccc1]

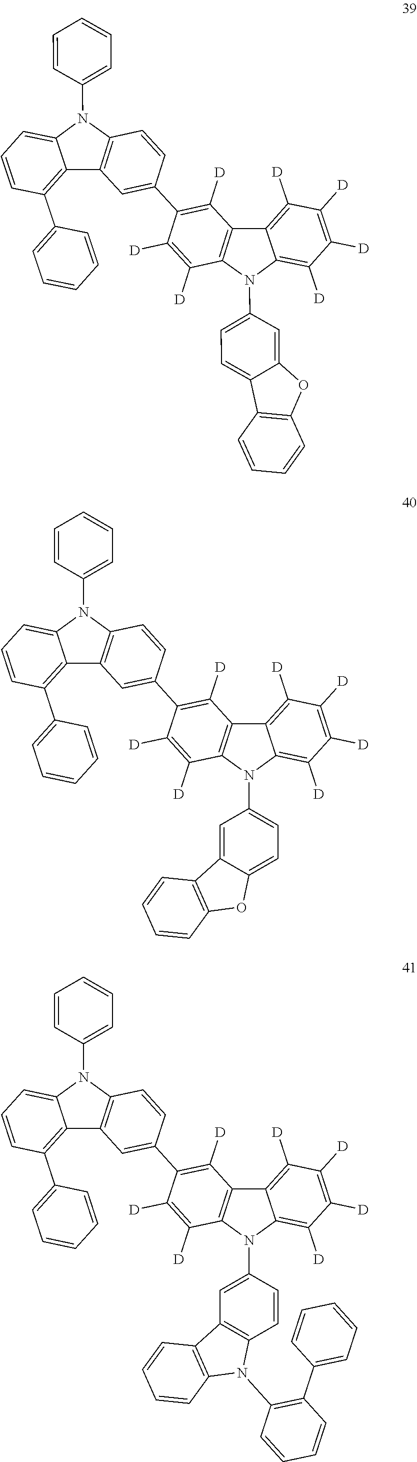 [SMILES: [2H]c1c([2H])c([2H])c2c(c1[2H])c1c([2H])c(-c3ccc4c(c3)c3c(-c5ccccc5)cccc3n4-c3ccccc3)c([2H])c([2H])c1n2-c1ccc2c(c1)oc1ccccc12 ; [2H]c1c([2H])c([2H])c2c(c1[2H])c1c([2H])c(-c3ccc4c(c3)c3c(-c5ccccc5)cccc3n4-c3ccccc3)c([2H])c([2H])c1n2-c1ccc2oc3ccccc3c2c1 ; [2H]c1c([2H])c([2H])c2c(c1[2H])c1c([2H])c(-c3ccc4c(c3)c3c(-c5ccccc5)cccc3n4-c3ccccc3)c([2H])c([2H])c1n2-c1ccc2c(c1)c1ccccc1n2-c1ccccc1-c1ccccc1]

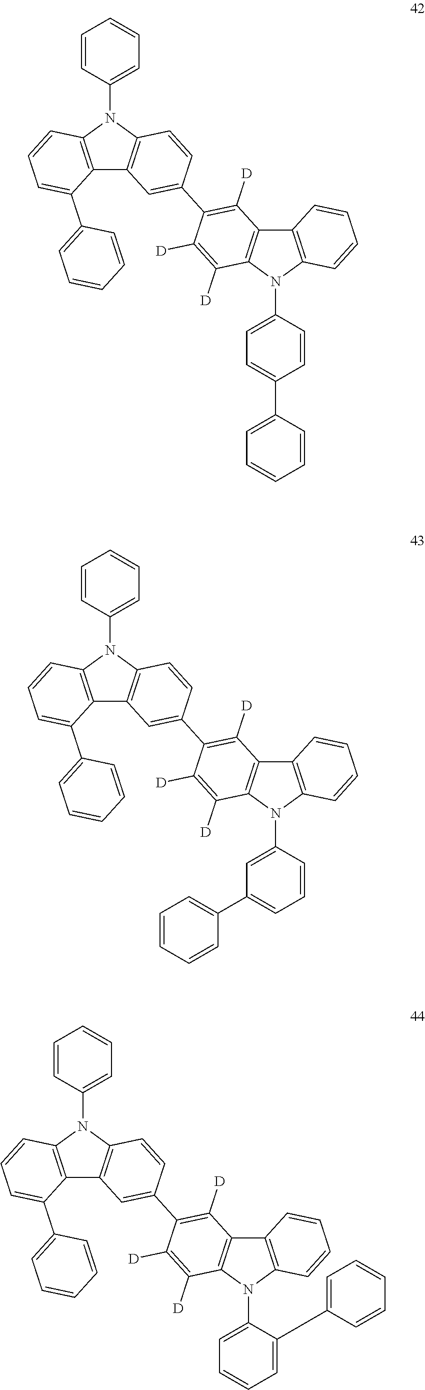 [SMILES: [2H]c1c(-c2ccc3c(c2)c2c(-c4ccccc4)cccc2n3-c2ccccc2)c([2H])c2c3ccccc3n(-c3ccc(-c4ccccc4)cc3)c2c1[2H] ; [2H]c1c(-c2ccc3c(c2)c2c(-c4ccccc4)cccc2n3-c2ccccc2)c([2H])c2c3ccccc3n(-c3cccc(-c4ccccc4)c3)c2c1[2H] ; [2H]c1c(-c2ccc3c(c2)c2c(-c4ccccc4)cccc2n3-c2ccccc2)c([2H])c2c3ccccc3n(-c3ccccc3-c3ccccc3)c2c1[2H]]

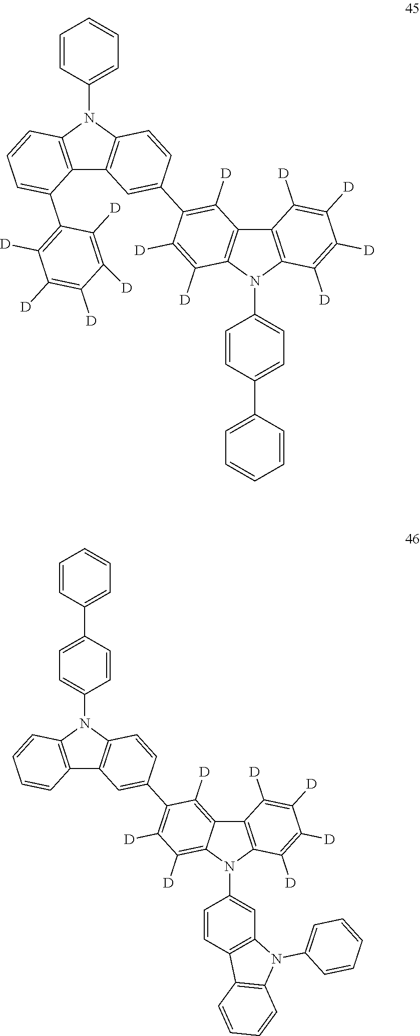 [SMILES: [2H]c1c([2H])c([2H])c(-c2cccc3c2c2cc(-c4c([2H])c([2H])c5c(c4[2H])c4c([2H])c([2H])c([2H])c([2H])c4n5-c4ccc(-c5ccccc5)cc4)ccc2n3-c2ccccc2)c([2H])c1[2H] ; [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1ccc2c3ccccc3n(-c3ccccc3)c2c1]

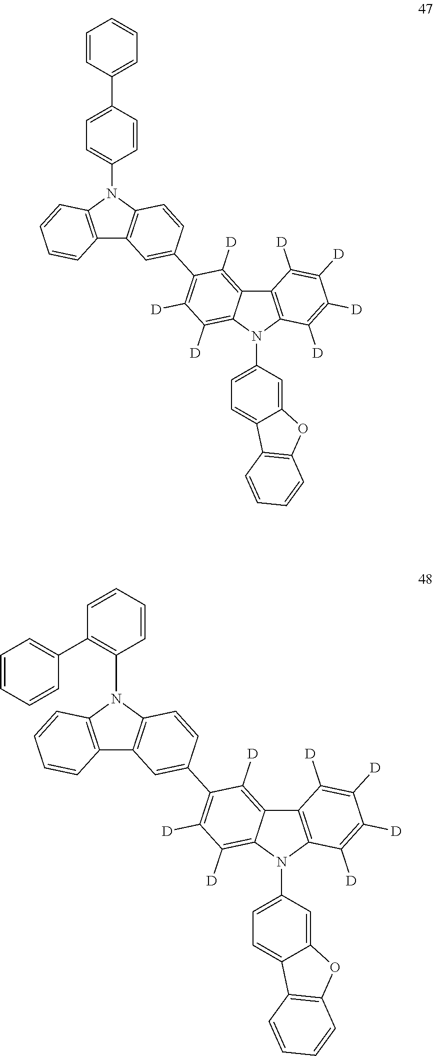 [SMILES: [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1ccc2c(c1)oc1ccccc12 ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1ccc2c(c1)oc1ccccc12]

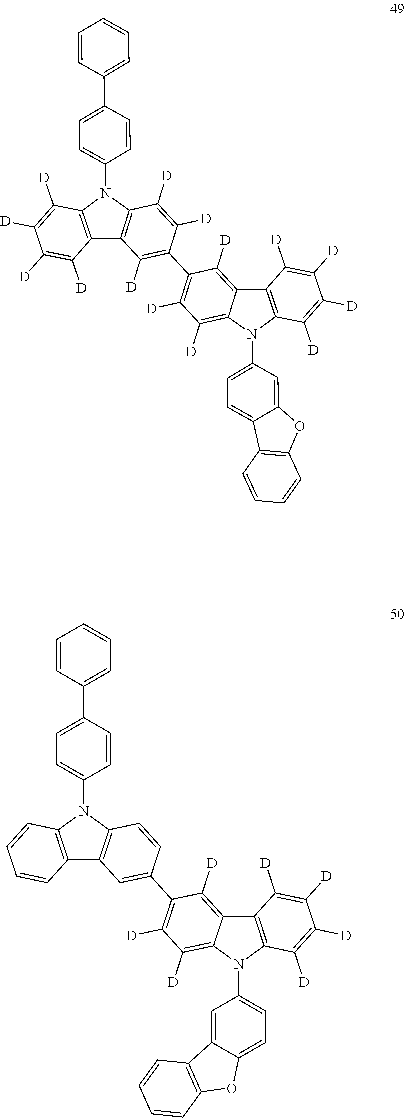 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3ccccc34)c([2H])c([2H])c1n2-c1ccc(-c2ccccc2)cc1 ; [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1ccc2oc3ccccc3c2c1]

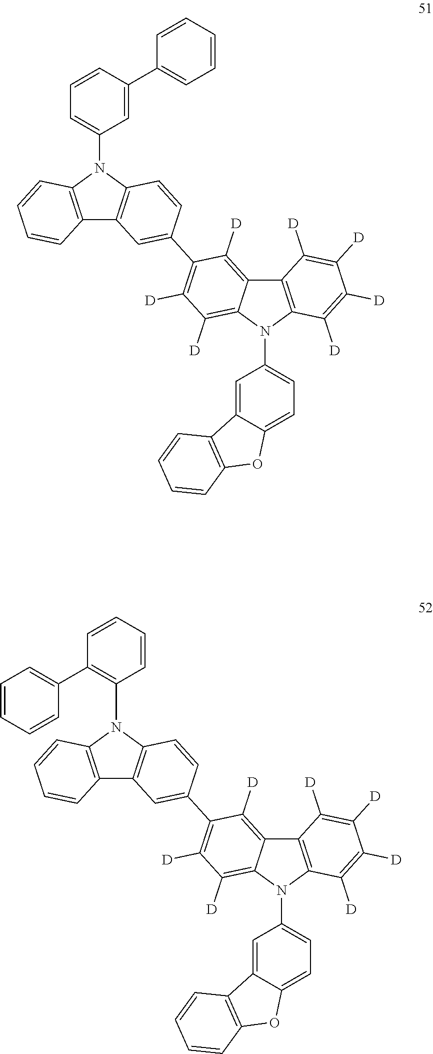 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2oc3ccccc3c2c1 ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1ccc2oc3ccccc3c2c1]

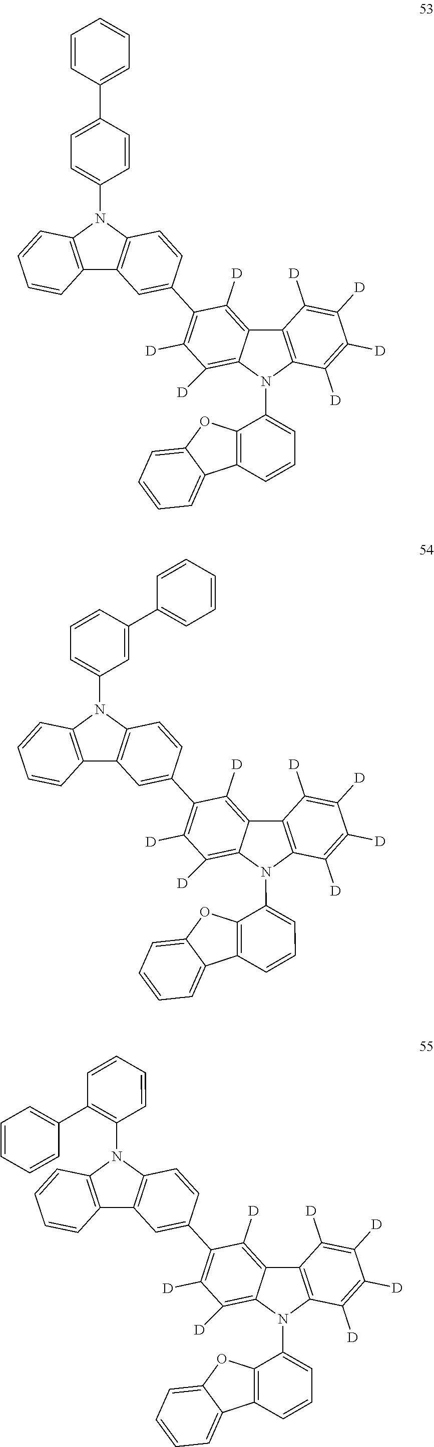 [SMILES: [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1cccc2c1oc1ccccc12 ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12 ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1cccc2c1oc1ccccc12]

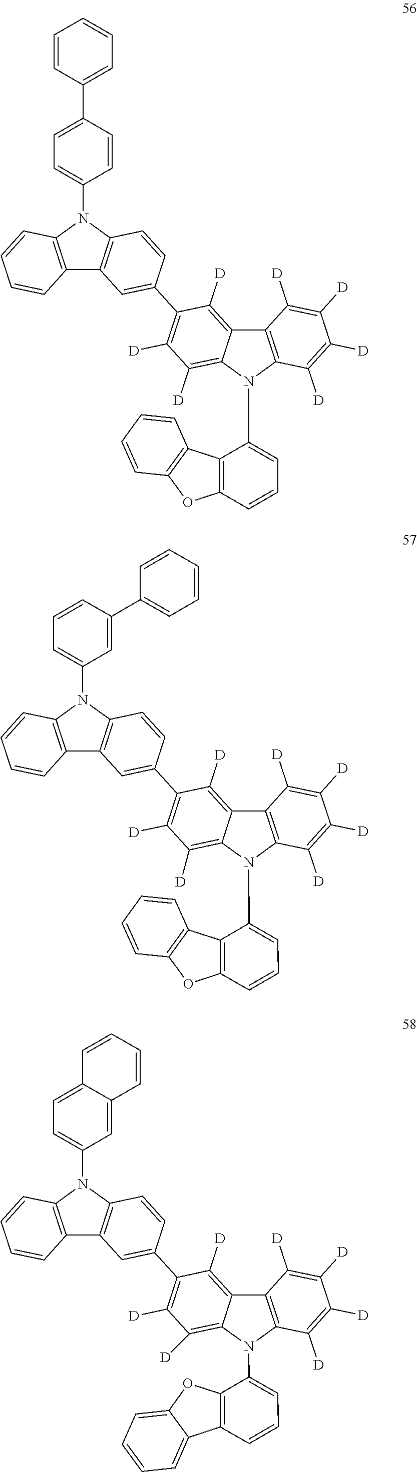 [SMILES: [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1cccc2oc3ccccc3c12 ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2oc3ccccc3c12 ; [2H]c1c([2H])c([2H])c2c(c1[2H])c1c([2H])c(-c3ccc4c(c3)c3ccccc3n4-c3ccc4ccccc4c3)c([2H])c([2H])c1n2-c1cccc2c1oc1ccccc12]

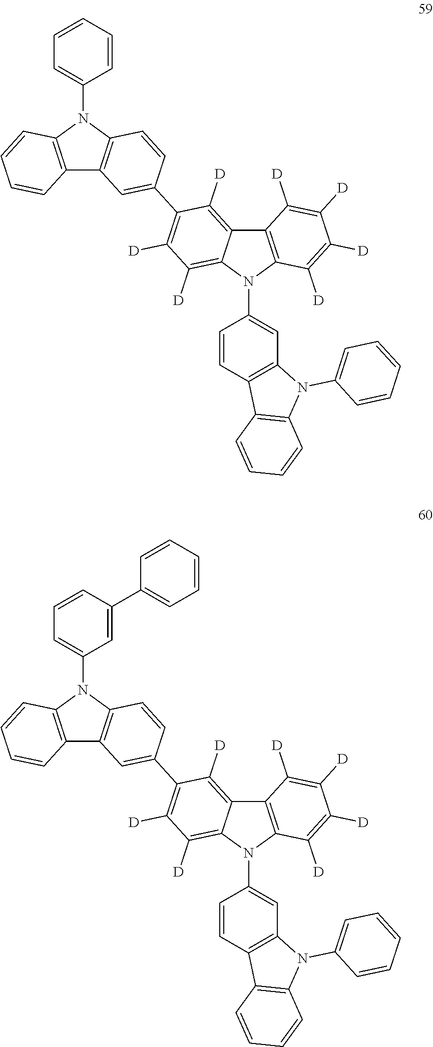 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccc2c3ccccc3n(-c3ccccc3)c2c1 ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2c3ccccc3n(-c3ccccc3)c2c1]

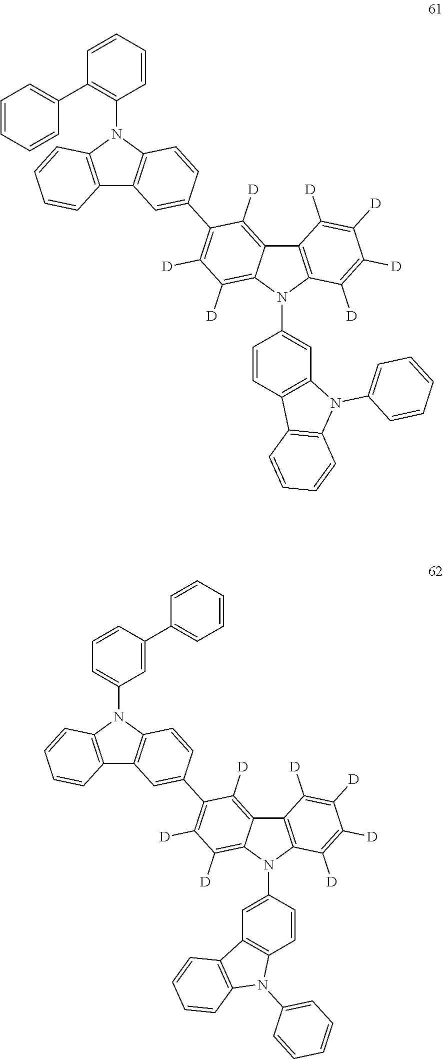 [SMILES: [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1ccc2c3ccccc3n(-c3ccccc3)c2c1 ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2c(c1)c1ccccc1n2-c1ccccc1]

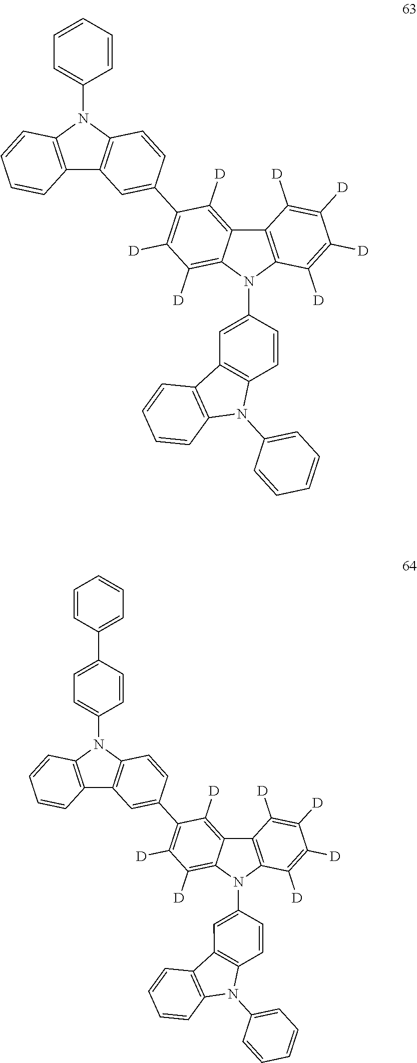 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccc2c(c1)c1ccccc1n2-c1ccccc1 ; [2H]c1c([2H])c([2H])c2c(c1[2H])c1c([2H])c(-c3ccc4c(c3)c3ccccc3n4-c3ccc(-c4ccccc4)cc3)c([2H])c([2H])c1n2-c1ccc2c(c1)c1ccccc1n2-c1ccccc1]

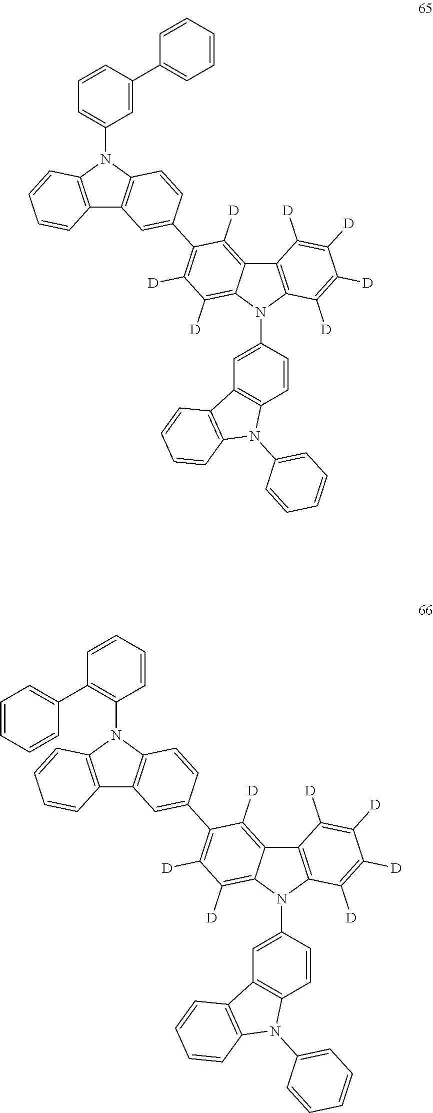 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2c(c1)c1ccccc1n2-c1ccccc1 ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1ccc2c(c1)c1ccccc1n2-c1ccccc1]

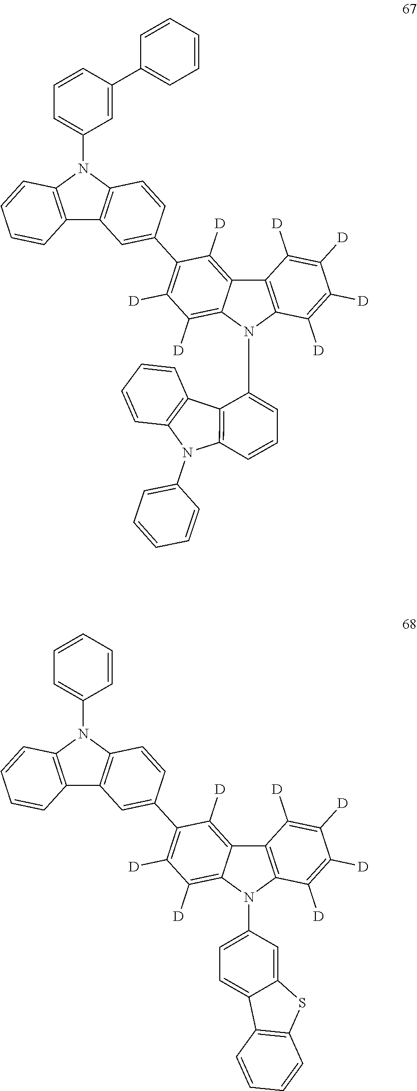 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1c1ccccc1n2-c1ccccc1 ; [2H]c1c([2H])c([2H])c2c(c1[2H])c1c([2H])c(-c3ccc4c(c3)c3ccccc3n4-c3ccccc3)c([2H])c([2H])c1n2-c1ccc2c(c1)sc1ccccc12]

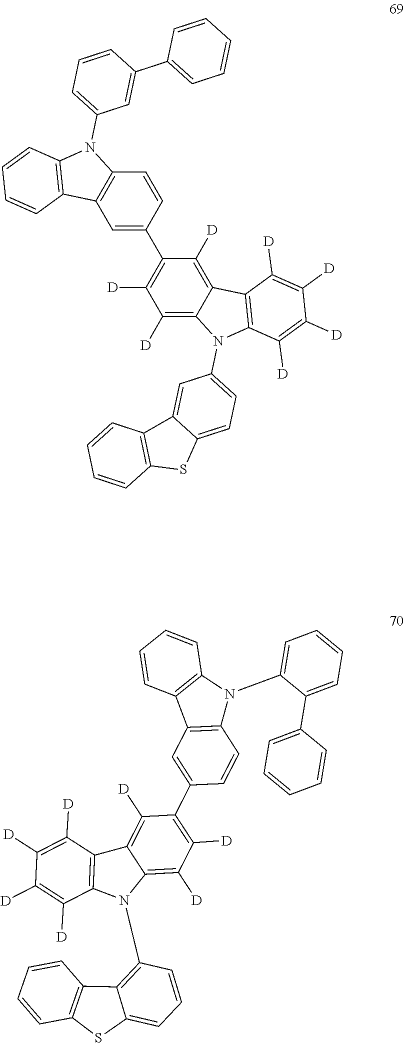 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2sc3ccccc3c2c1 ; [2H]c1c([2H])c([2H])c2c(c1[2H])c1c([2H])c(-c3ccc4c(c3)c3ccccc3n4-c3ccccc3-c3ccccc3)c([2H])c([2H])c1n2-c1cccc2sc3ccccc3c12]

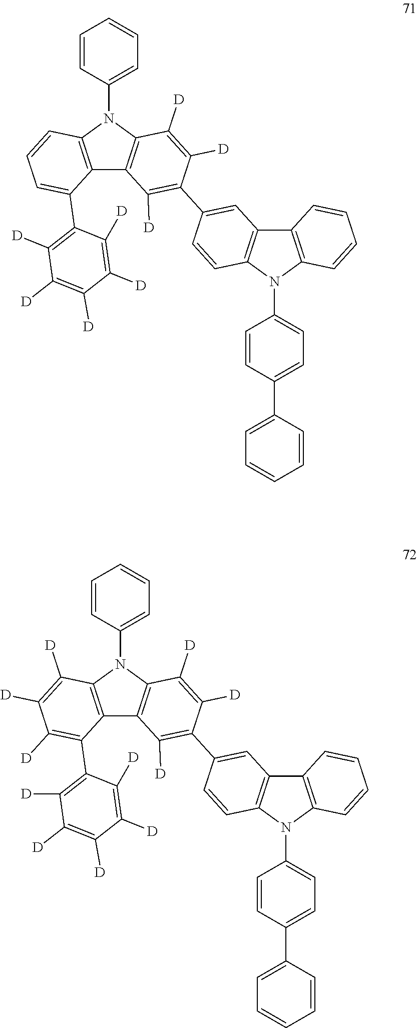 [SMILES: [2H]c1c([2H])c([2H])c(-c2cccc3c2c2c([2H])c(-c4ccc5c(c4)c4ccccc4n5-c4ccc(-c5ccccc5)cc4)c([2H])c([2H])c2n3-c2ccccc2)c([2H])c1[2H] ; [2H]c1c([2H])c([2H])c(-c2c([2H])c([2H])c([2H])c3c2c2c([2H])c(-c4ccc5c(c4)c4ccccc4n5-c4ccc(-c5ccccc5)cc4)c([2H])c([2H])c2n3-c2ccccc2)c([2H])c1[2H]]

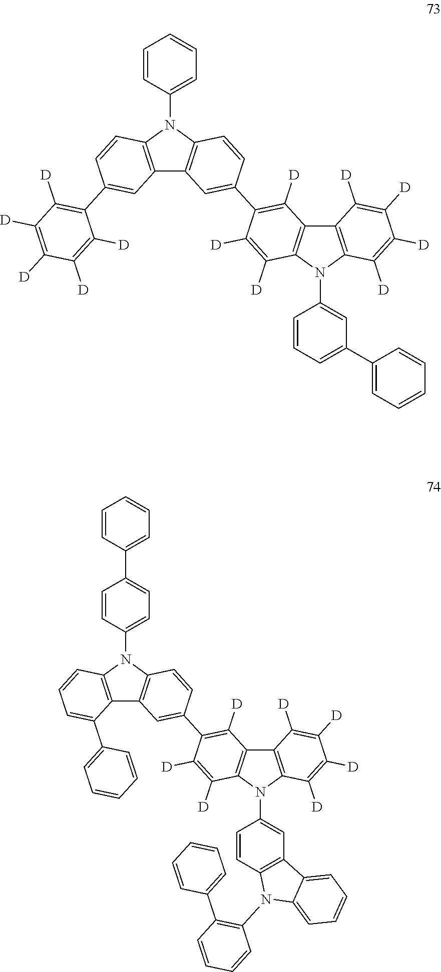 [SMILES: [2H]c1c([2H])c([2H])c(-c2ccc3c(c2)c2cc(-c4c([2H])c([2H])c5c(c4[2H])c4c([2H])c([2H])c([2H])c([2H])c4n5-c4cccc(-c5ccccc5)c4)ccc2n3-c2ccccc2)c([2H])c1[2H] ; [2H]c1c([2H])c([2H])c2c(c1[2H])c1c([2H])c(-c3ccc4c(c3)c3c(-c5ccccc5)cccc3n4-c3ccc(-c4ccccc4)cc3)c([2H])c([2H])c1n2-c1ccc2c(c1)c1ccccc1n2-c1ccccc1-c1ccccc1]

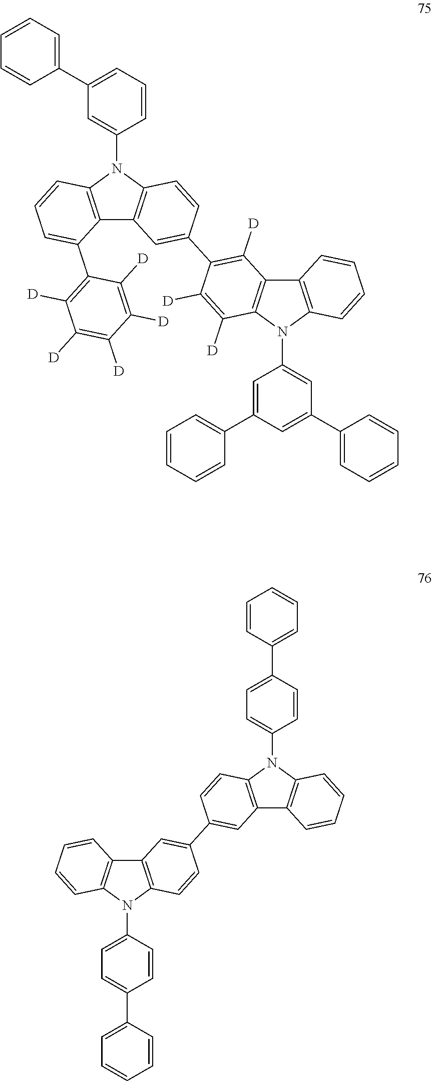 [SMILES: [2H]c1c([2H])c([2H])c(-c2cccc3c2c2cc(-c4c([2H])c([2H])c5c(c4[2H])c4ccccc4n5-c4cc(-c5ccccc5)cc(-c5ccccc5)c4)ccc2n3-c2cccc(-c3ccccc3)c2)c([2H])c1[2H] ; c1ccc(-c2ccc(-n3c4ccccc4c4cc(-c5ccc6c(c5)c5ccccc5n6-c5ccc(-c6ccccc6)cc5)ccc43)cc2)cc1]

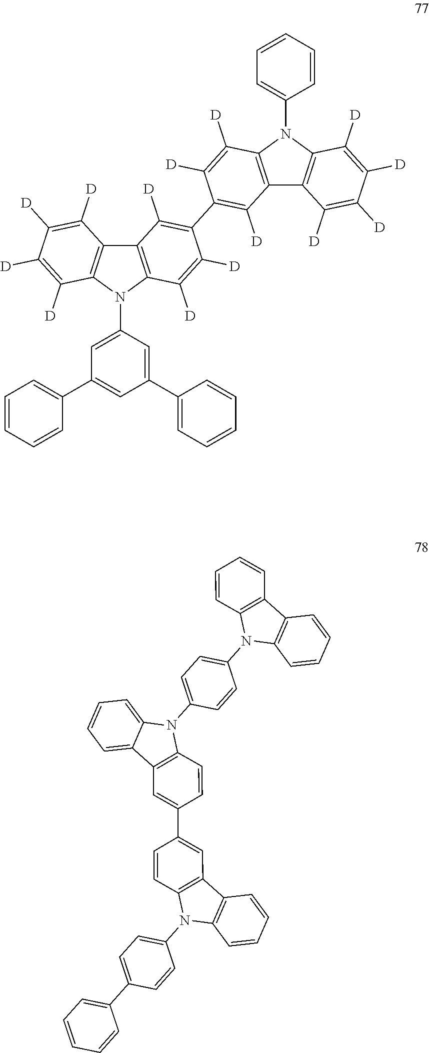 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1ccccc1 ; c1ccc(-c2ccc(-n3c4ccccc4c4cc(-c5ccc6c(c5)c5ccccc5n6-c5ccc(-n6c7ccccc7c7ccccc76)cc5)ccc43)cc2)cc1]

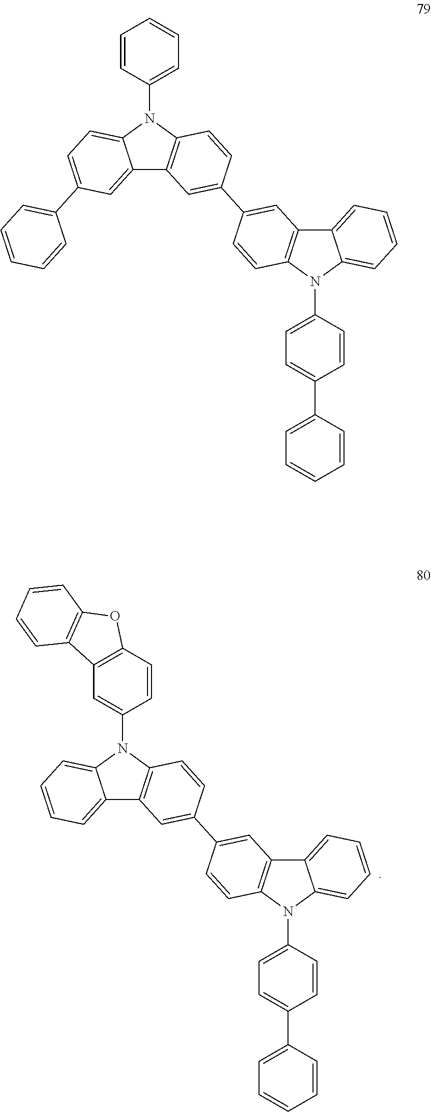 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

14. An organic electroluminescent device, comprising an anode and a cathode arranged oppositely, as well as a functional layer arranged between the anode and the cathode; wherein
the functional layer comprises the organic compound according to claim 1;
optionally, the functional layer comprises an organic light-emitting layer, and the organic light-emitting layer comprises the organic compound;
optionally, the organic electroluminescent device is a green organic electroluminescent device.

15. An electronic apparatus, comprising the organic electroluminescent device according to claim 14.

16. An organic electroluminescent device, comprising an anode and a cathode arranged oppositely, as well as a functional layer arranged between the anode and the cathode; wherein
the functional layer comprises the composition according to claim 9;
optionally, the functional layer comprises an organic light-emitting layer, and the organic light-emitting layer comprises the composition; and
optionally, the organic electroluminescent device is a green organic electroluminescent device.

17. An electronic apparatus, comprising the organic electroluminescent device according to claim 16.
